(19) United States
(12) Patent Application Publication
(10) Pub. No.: US 2023/0357253 A1
(43) Pub. Date: Nov. 9, 2023

(54) AGONISTS OF STIMULATOR OF INTERFERON GENES STING

(71) Applicant: THE SCRIPPS RESEARCH INSTITUTE, La Jolla, CA (US)

(72) Inventors: Hank Michael James Petrassi, San Diego, CA (US); Luke L. Lairson, San Diego, CA (US); Emily Chin, San Diego, CA (US); Peter G. Schultz, La Jolla, CA (US); Chenguang Yu, San Diego, CA (US); Baiyuan Yang, San Diego, CA (US); Virginia Grant, San DIego, CA (US); Yongkai Li, Cardiff by the Sea, CA (US); Alexander Pacheco, San Diego, CA (US); Alan Chu, San Diego, CA (US); Kristen Johnson, Santee, CA (US); Arnab K. Chatterjee, San Diego, CA (US)

(21) Appl. No.: 18/043,649

(22) PCT Filed: Sep. 2, 2021
(86) PCT No.: PCT/US2021/071355

§ 371 Date: Mar. 1, 2023

Related U.S. Application Data
(60) Provisional application No. 62/706,683, filed on Sep. 2, 2020.

(51) Int. Cl. C07D 487/04; C07D 519/00; C07D 403/14; C07D 413/14; C07F 9/6558; C07D 409/14; C07D 405/14; C07D 401/14; C07D 237/24; A61K 45/06

(52) U.S. Cl. CPC C07D 487/04; C07D 519/00; C07D 403/14; C07D 413/14; C07F 9/65583; C07D 409/14; C07D 405/14; C07D 401/14; C07D 237/24; A61K 45/06

(57) ABSTRACT

Disclosed herein are compounds of Formula (I), pharmaceutically acceptable salts thereof, and their pharmaceutical compositions: The compounds are useful as agonists of Stimulator of Interferon Genes (STING), such as in a method of treating a tumor.

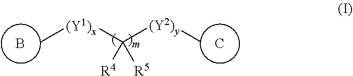

This application claims the benefit of priority to U.S. Provisional Patent Application No. 62/706,683, filed on Sep. 2, 2020, and which application is incorporated as if fully set forth herein.

### BACKGROUND

The cGAS-STING signaling pathway plays a critical role in the innate immune response that mammalian host cells mount to eliminate diverse DNA and RNA viruses (Q. Chen. L. Sun, Z. J. Chen, Nat. Immunol. 17, 1142-1149 (2016); M. H. Christensen. S. R. Paludan, Cell. Mol. Immunol. 14, 4-13 (2017)). STING (Stimulator of Interferon Genes) is an endoplasmic reticulum (ER) resident signaling protein, partially localized to mitochondria-associated membranes, which is broadly expressed in both immune and non-immune cell types. STING also serves as a direct link between inflammation and diverse physiological processes, including micronuclei surveillance in the context of DNA damage (K. J. Mackenzie et al., Nature 548, 461-465 (2017); S. M. Harding et al., Nature 548, 466-470 (2017)), age-associated inflammation (De Cecco et al., Nature 566, 73-78 (2019)), mitochondrial DNA-related inflammatory phenotypes (D. A. Sliter et al., Nature 561, 258-262 (2018)), and microbiome-dependent intestinal homeostasis (M. C. C. Canesso et al., Mucosal Immunol. 11,820-834 (2018)). STING is an endoplasmic reticulum signaling protein, partially localized to mitochondria-associated membranes, that is broadly expressed in both immune and nonimmune cell types. STING binds cyclic dinucleotides (CDNs)—including 2′,3′-cyclic GMP-AMP (2′,3′-cGAMP) produced by cGAS in response to cytosolic DNA (L. Sun, J. Wu, F. Du, X. Chen, Z. J. Chen, Science 339, 786-791 (2013))—and the scaffolding function rapidly induces type I interferon (IFN) and proinflammatory cytokines in a TBK1-IRF3-dependent fashion (H. Ishikawa. Z. Ma, G. N. Barber, Nature 461,788-792 (2009); H. Ishikawa, G. N. Barber, Nature 455, 674-678 (2008)).

STING is demonstrated to play essential roles in antitumor immunity. For example, efficient tumor-initiated T cell activation requires STING pathway-dependent IFN-β expression, as well as expression of STING in dendritic cells (DCs) (M. B. Fuertes et al., J. Exp. Med. 208, 2005-2016 (2011); S. R. Woo et al., Immunity 41, 830-842 (2014)).

Initial STING agonist small molecules were synthesized as derivatives of the CDN natural ligand. Because of poor stability properties, however, CDN-based agonist administration is limited to intratumoral delivery. Although intratumoral delivery of CDN agonists has consistently shown regression of established tumors in syngeneic models (Corrales et al., Cell Rep. 11, 1018-1030 (2015); K. E. Sivick et al., Cell Rep. 29, 785-789 (2019)), intra-tumor CDN administration in humans has been met with mixed success.

Activation of the STING pathway also is demonstrated to contribute notably to the antitumor effect of radiation and chemotherapeutics (Harding et al. (2017), C. Vanpouille-Box et al., Nat. Commun. 8, 15618 (2017); C. Pantelidou et al., Cancer Discov. 9, 722-737 (2019)).

### SUMMARY

In various embodiments, the present disclosure provides an agonist of the Stimulator of Interferon Genes (STING), which can be used in the treatment of tumors. According to various embodiments, the agonist is a compound of formula (I) or a pharmaceutically acceptable salt thereof:

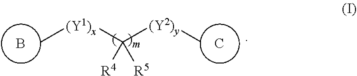

Rings B and C are independently selected from Het, formula (a) and formula (b):

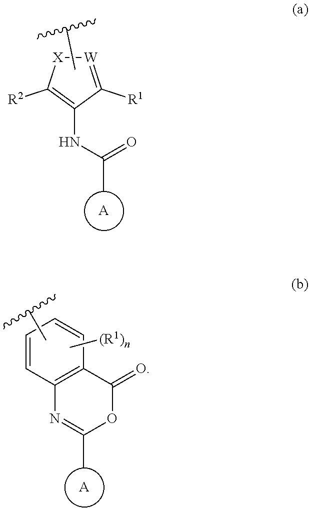

Each ring A is optionally substituted by 1 to 4 R^{A} and is independently selected from a 5- or 6-membered monocyclic heteroaryl comprising 1 to 3 heteroatoms selected from O, S, and N, and an 8- to 10-membered bicyclic heteroaryl comprising 1 to 6 heteroatoms selected from O, S, and N.

Het is an 8- to 10-membered bicyclic heteroaryl comprising 1 to 6 heteroatoms selected from O, S, and N and that is optionally substituted by 1 to 4 R^{A}.

X is N, S, —N═C(R¹)—, or —C(R³)═C(R³)—.

W is —N═ or —C(R³)═.

Y¹ is selected from —O—, —CR₄R₅—, —(CH₂)_{L1}—O—, —(CH₂)_{L1}—S(O)₀₋₂— (wherein L1 is an integer selected from 1, 2, 3, 4, and 5); and —(CH₂)_{L1}—N(R^{L})— (wherein R^{L} is selected from H. C₁-C₆-alkyl, and benzyl optionally substituted by 1 or 2 methoxy).

Y² is selected from —O—, —CR₄R₅—, —O—(CH₂)_{L1}—, —S(O)₀₋₂—(CH₂)_{L1}— (wherein L1 is an integer selected from 1, 2, 3, 4, and 5); and —N(R^{L})—(CH₂)_{L1}— (wherein R^{L} is H or C₁₂-C₆-alkyl).

Subscript m is an integer selected from 0, 1, 2, 3, 4, 5, and 6.

Subscript n is an integer selected from 0, 1, and 2.

Subscripts x and y are integers independently selected from 0 and 1, wherein Y¹ and Y² are not simultaneously —O— when m is 0 and each of x and y is 1.

Each R¹ and R³ is independently selected from the group consisting of H, halo, C₁-C₆-alkyl, C₂-C₆-alkenyl, C₂-C₆-alkynyl, C₁-C₆-alkoxyl, cyano, C₁-C₆-haloalkyl, and 3- to 10-membered heterocyclyl (wherein 1-4 heterocycloalkyl members are independently selected from N, O, and S), wherein any alkyl, alkenyl, alkynyl, alkoxyl, or heterocyclyl is optionally substituted by 1 to 4 R^{A}.

R² is selected from the group consisting of —C(O)OR, —(C₁-C₆-alkyl)C(O)OR, C₁-C₆-haloalkyl, —P(O)(OR)₂, —C(O)NHR, halo, —CN, C₃-C₆-cycloalkenyl, 3- to 10-membered heterocyclyl (wherein 1-4 heterocycloalkyl members are independently selected from N, O, and S), and 5- to 10-membered heteroaryl (wherein 1-4 heteroaryl members are independently selected from N, O, and S), wherein any alkyl, cycloalkenyl, heterocyclyl, or heteroaryl is optionally substituted by 1 to 4 R^{A}.

R is selected from the group consisting of H; C₁-C₆-alkyl optionally substituted with —((C₁-C₆-alkyl)OC(O)OC₁-C₆-alkyl), —OP(O)(OH)₂, —OC(O)(C₁-C₆-alkyl)-O—P(O)(OH)₂, —NH₂, —CH(NH₂)COOH, or 3- to 10-membered heterocyclyl (wherein 1-4 heterocycloalkyl members are independently selected from N, O, and S); and —(C₁-C₆-alkyl)(C₆-C₁₀-aryl).

Each R⁴ and R⁵ is independently selected from the group consisting of H, halo, C₁-C₆-alkyl, and C₃-C₇-cycloalkyl. In some embodiments, any two R⁴ and R⁵ bound to the same carbon atom, together with the carbon atom to which they are bound, represent a C₃-C₅-cycloalkyl optionally substituted by 1 to 3 R^{A}, or they represent a C₂-C₆-alkenyl. In still other embodiments, any two of R⁴ and R⁵ not bound to the same carbon atom, together with the respective carbon atoms to which they are bound, represent a C₃-C₇-cycloalkyl optionally substituted by 1 to 3 R^{A}.

Each instance of R^{A} is independently selected from the group consisting of H, halo, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₁-C₆-alkyl)COOH, —C(O)(C₁-C₆-alkyl)C(O)(C₁-C₆-alkoxy), —C(O)N(H or C₁-C₆-alkyl)₂, —C(O)(C₃-C₁₄-cycloalkyl), —C₃-C₄-cycloalkyl, —(C₁-C₆-alkyl)(C₃-C₁₄-cycloalkyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S) that is optionally substituted with C₁-C₆-alkyl.

More specifically, in illustrative embodiments, a compound or pharmaceutically acceptable salt thereof according to the present disclosure includes any of the specific compounds shown in Table 1 or Table 2 below.

The present disclosure also provides in various embodiments a pharmaceutical composition comprising a compound or pharmaceutically acceptable salt thereof as disclosed herein and a pharmaceutically acceptable carrier.

The present disclosure also provides in an embodiment a method of stimulating expression of interferon genes, comprising administering to a patient an effective amount of an agonist of the Stimulator of Interferon Genes (STING), comprising a compound as described herein, and a method of treating a tumor in a patient, comprising administering to the patient an effective amount of an agonist of the Stimulator of Interferon Genes (STING), comprising a compound of formula (I).

In various embodiments, the method of treatment of a tumor further comprises administering an effective dose of a compound as disclosed herein via oral or intratumoral administration, or both.

In various embodiments, the method of treatment of a tumor further comprises administering an effective amount of a compound as disclosed herein, wherein administering comprises administering the compound to the patient as an antibody-drug conjugate, or in a liposomal formulation.

In various embodiments, the method of treatment of a tumor further comprises administering an effective amount of a compound as disclosed herein, further comprising administration of an effective dose of an immune-checkpoint targeting drug. For example, the immune-checkpoint targeting drug can be an anti-PD-L1 antibody, anti-PD-1 antibody, anti-CTLA-4 antibody, or an anti-4-1BB antibody.

In various embodiments, the method of treatment of a tumor further comprises administering an effective amount of a compound as disclosed herein, further comprising administration of ionizing radiation or anticancer drugs.

### DETAILED DESCRIPTION

Significant interest resides in the development of STING pathway agonists for diverse immuno-oncology applications. Most notably, STING pathway agonists have significant potential application as part of combination therapies involving immune-checkpoint targeting drugs, in patients that fail to respond to checkpoint blockade alone. Accordingly, a systemic STING-activating agent has considerable utility not only as a therapeutic for cancer and infectious disease, but also as a pharmacological probe to enable mechanistic discovery in the context of STING-dependent antitumor immunity and diverse STING-dependent biological processes. The present disclosure addresses these needs and others in the provision of STING agonist compounds and pharmaceutically acceptable salts, their pharmaceutical compositions, and their methods of use.

The present disclosure relates in part to non-nucleotide small molecule STING agonists, whose activity is established through a primary assay involving a human THP-1 cell line carrying an IRF-inducible reporter with 5 copies of the IFN signaling response element. Counter screens, involving alternative reporter constructs, rodent cell-based assays, as well as cGAS and STING knock-out cell lines, are used to eliminate luciferase artifacts, to ensure human-rodent cross species reactivity, and to ensure pathway selectivity. Biochemical assays, involving cGAS enzymatic activity and STING protein binding assays, are used to identify the specific target of identified hits.

### Definitions

Standard abbreviations for chemical groups such as are well known in the art are used; e.g., Me=methyl, Et=ethyl, i-Pr=isopropyl, Bu=butyl, t-Bu=tert-butyl. Ph=phenyl, Bn=benzyl, Ac=acetyl, Bz=benzoyl, and the like.

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂—CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

The term “alkenyl” refers to straight or branched chain hydrocarbyl groups including from 2 to about 20 carbon atoms, such as 2 to 6 carbon atoms, and having 1-3, 1-2, or at least one carbon to carbon double bond. An alkenyl group can be unsubstituted or optionally substituted with one or more substituents as described herein.

“Substituted alkenyl” refers to alkenyl substituted at 1 or more, e.g., 1, 2, 3, 4, 5, or even 6 positions, which substituents are attached at any available atom to produce a stable compound, with substitution as described herein. “Optionally substituted alkenyl” refers to alkenyl or substituted alkenyl.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

“Substituted alkynyl” refers to an alkynyl substituted at 1 or more, e.g., 1, 2, 3, 4, 5, or even 6 positions, which substituents are attached at any available atom to produce a stable compound, with substitution as described herein. “Optionally substituted alkynyl” refers to alkynyl or substituted alkynyl.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

Aryl groups are cyclic aromatic hydrocarbons that do not contain heteroatoms in the ring. An aromatic compound, as is well-known in the art, is a multiply-unsaturated cyclic system that contains 4n+2 π electrons where n is an integer. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups (see e.g. Lang's Handbook of Chemistry (Dean, J. A., ed) 13^{th} ed. Table 7-2 [1985]). In some embodiments, aryl groups contain the number of carbon atoms designated or if no number is designated, up to 14 carbon atoms, such as a C₆-C₁₄-aryl. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

The term “heteroatom” refers to N, O, and S atoms. Compounds of the present disclosure that contain N or S atoms can be optionally oxidized to the corresponding N-oxide, sulfoxide, or sulfone compounds.

Heterocyclyl groups or the term “heterocyclyl” includes aromatic and non-aromatic ring compounds containing 3 or more ring members, of which one or more ring atom is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 14 ring members. A heterocyclyl group designated as a C2-heterocyclyl can be a 5-ring with two carbon atoms and three heteroatoms, a 6-ring with two carbon atoms and four heteroatoms and so forth. Likewise, a C4-heterocyclyl can be a 5-ring with one heteroatom, a 6-ring with two heteroatoms, and so forth. The number of carbon atoms plus the number of heteroatoms sums up to equal the total number of ring atoms. Ring sizes can also be expressed by the total number of atoms in the ring, e.g., a 3- to 10-membered heterocyclyl group, counting both carbon and non-carbon ring atoms. A heterocyclyl ring can also include one or more double bonds. A heteroaryl ring is an embodiment of a heterocyclyl group. The term “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The term also includes polycyclic, e.g., bicyclo- and tricyclo-ring systems containing one or more heteroatom such as, but not limited to, quinuclidyl.

“Optionally substituted heterocycloalkyl” denotes a heterocycloalkyl that is substituted with 1 to 3 substituents, e.g., 1, 2 or 3 substituents, attached at any available atom to produce a stable compound, wherein the substituents are as described herein.

Heteroaryl groups are heterocyclic aromatic ring compounds containing 5 or more ring members, of which, one or more is a heteroatom such as, but not limited to, N, O, and S; for instance, heteroaryl rings can have 5 to about 8-12 ring members, such as a 5- to 10-membered heteroaryl. Some bicyclic heteroaryl rings can have 8- to 10 ring members. A heteroaryl group is a variety of a heterocyclyl group that possesses an aromatic electronic structure, which is a multiply-unsaturated cyclic system that contains 4n+2 π electrons wherein n is an integer. A heteroaryl group designated as a C2-heteroaryl can be a 5-ring (i.e., a 5-membered ring) with two carbon atoms and three heteroatoms, a 6-ring (i.e., a 6-membered ring) with two carbon atoms and four heteroatoms and so forth. Likewise, a C4-heteroaryl can be a 5-ring with one heteroatom, a 6-ring with two heteroatoms, and so forth. The number of carbon atoms plus the number of heteroatoms sums up to equal the total number of ring atoms. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

Cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

Cycloalkenyl groups include cycloalkyl groups having at least one double bond between 2 carbons. Thus, for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

The term “oxo” refers to a ═O atom bound to an atom that is part of a saturated or unsaturated moiety. Thus, for example, the ═O atom can be bound to a carbon, sulfur, or nitrogen atom that is part of a cyclic or acyclic moiety.

One or more optional substituents on any group described herein are independently selected from the group consisting of R^{A}, OR^{A}, halo, —N═N—R^{A}, NR^{A}R^{B}, —(C₁-C₆-alkyl)NR^{A}R^{B}, —C(O)OR^{A}, —C(O)NR^{A}R^{B}, —OC(O)R^{A}, and —CN. R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S). Each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl described above is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

The substituent —CO₂H may be replaced with bioisosteric replacements such as:

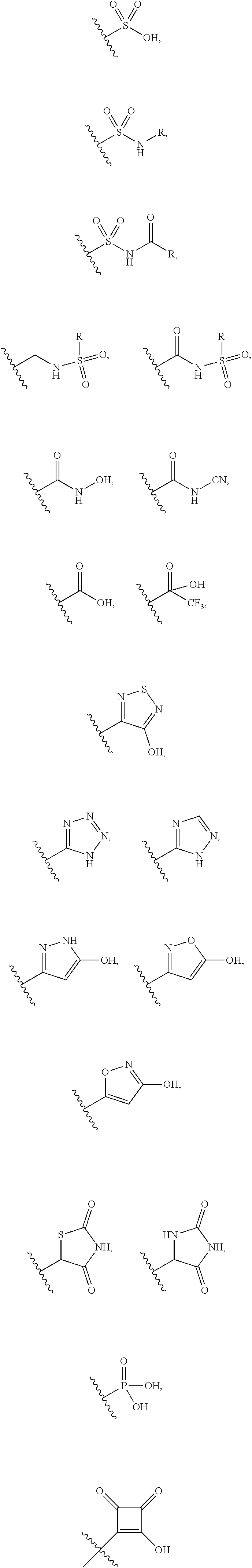

and the like, wherein R has the same definition as R^{A} as defined herein. See, e.g., THE PRACTICE OF MEDICINAL CHEMISTRY (Academic Press: New York, 1996), at page 203.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of the present disclosure includes a pharmaceutically acceptable salt of a tautomer of the compound.

The term “pharmaceutically acceptable salts” refers to nontoxic inorganic or organic acid and/or base addition salts, see, for example, Lit, et al., Salt Selection for Basic Drugs (1986), Int J. Pharm., 33, 201-217, incorporated by reference herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. Also included are amino acid salts, such as cysteine salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the present disclosure refers to an amount of the compound that alleviates, in whole or in part, symptoms associated with the disorder or condition, or halts or slows further progression or worsening of those symptoms, or prevents, or provides prophylaxis for, the disorder or condition. For example, a “therapeutically effective amount” refers to an amount that is effective, at dosages and for periods of time necessary, to achieve the desired therapeutic result. A therapeutically effective amount is also one in which any toxic or detrimental effects of compounds of the present disclosure are outweighed by the therapeutically beneficial effects.

The expression “effective amount”, when used to describe therapy to an individual suffering from a disorder, refers to the quantity or concentration of a compound of the present disclosure that is effective to activate or otherwise act on STING in the individual's tissues wherein STING involved in the disorder, wherein such activation or other action occurs to an extent sufficient to produce a beneficial therapeutic effect. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

Generally, the initial therapeutically effective amount of a compound described herein or a pharmaceutically acceptable salt thereof that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of the compound or a pharmaceutically acceptable salt thereof. In another embodiment, such dosage forms contain from about 50 mg to about 500 mg of the compound or a pharmaceutically acceptable salt thereof. In yet another embodiment, such dosage forms contain from about 25 mg to about 200 mg of the compound or a pharmaceutically acceptable salt thereof. In still another embodiment, such dosage forms contain from about 10 mg to about 100 mg of the compound or a pharmaceutically acceptable salt thereof. In a further embodiment, such dosage forms contain from about 5 mg to about 50 mg of the compound or a pharmaceutically acceptable salt thereof. In any of the foregoing embodiments the dosage form can be administered once a day or twice per day.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

Compounds

The present disclosure provides in various embodiments a compound of formula (I) or a pharmaceutically acceptable salt thereof:

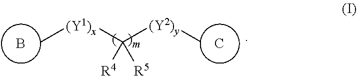

Rings B and C are independently selected from Het, formula (a) and formula (b):

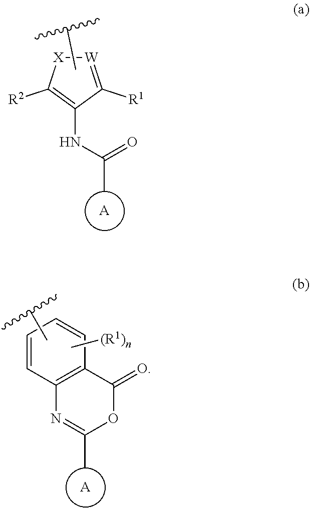

Each ring A is optionally substituted by 1 to 4 R^{A} and is independently selected from a 5- or 6-membered monocyclic heteroaryl comprising 1 to 3 heteroatoms selected from 0, S, and N, and an 8- to 10-membered bicyclic heteroaryl comprising 1 to 6 heteroatoms selected from O, S, and N.

Het is an 8- to 10-membered bicyclic heteroaryl comprising 1 to 6 heteroatoms selected from O, S, and N and that is optionally substituted by 1 to 4 R^{A}.

X is N, S, —N═C(R¹)—, or —C(R³)═C(R³)—.

W is —N═ or —C(R³)═.

Y¹ is selected from —O—, —CR₄R₅—, —(CH₂)_{L1}—O—, —(CH₂)_{L1}—S(O)₀₋₂— (wherein L1 is an integer selected from 1, 2, 3, 4, and 5); and —(CH₂)_{L1}—N(R^{L})— (wherein R^{L} is selected from H, C₁-C₆-alkyl, and benzyl optionally substituted by 1 or 2 methoxy).

Y² is selected from —O—, —CR₄R₅—, —O—(CH₂)_{L1}—, —S(O)₀₋₂—(CH₂)_{L1}— (wherein L1 is an integer selected from 1, 2, 3, 4, and 5); and —N(R^{L})—(CH₂)_{L1}— (wherein R^{L} is H or C₁₂-C₆-alkyl).

Subscript m is an integer selected from 0, 1, 2, 3, 4, 5, and 6.

Subscript n is an integer selected from 0, 1, and 2.

Subscripts x and y are integers independently selected from 0 and 1, wherein Y¹ and Y² are not simultaneously —O— when m is 0 and each of x and y is 1.

Each R¹ and R³ is independently selected from the group consisting of H, halo, C₁-C₆-alkyl, C₂-C₆-alkenyl, C₂-C₆-alkynyl, C₁-C₆-alkoxyl, cyano, C₁-C₆-haloalkyl, and 3- to 10-membered heterocyclyl (wherein 1-4 heterocycloalkyl members are independently selected from N, O, and S), wherein any alkyl, alkenyl, alkynyl, alkoxyl, or heterocyclyl is optionally substituted by 1 to 4 R^{A}.

R² is selected from the group consisting of —C(O)OR, —(C₁-C₆-alkyl)C(O)OR, C₁-C₆-haloalkyl, —P(O)(OR)₂, —C(O)NHR, halo, —CN, C₃-C₆-cycloalkenyl, 3- to 10-membered heterocyclyl (wherein 1-4 heterocycloalkyl members are independently selected from N, O, and S), and 5- to 10-membered heteroaryl (wherein 1-4 heteroaryl members are independently selected from N, O, and S), wherein any alkyl, cycloalkenyl, heterocyclyl, or heteroaryl is optionally substituted by 1 to 4 R^{A}.

R is selected from the group consisting of H; C₁-C₆-alkyl optionally substituted with —((C₁-C₆-alkyl)OC(O)OC₁-C₆-alkyl), —OP(O)(OH)₂, —OC(O)(C₁-C₆-alkyl)-O—P(O)(OH)₂, —NH₂, —CH(NH₂)COOH, or 3- to 10-membered heterocyclyl (wherein 1-4 heterocycloalkyl members are independently selected from N, O, and S); and —(C₁-C₆-alkyl)(C₁-C₁₀-aryl).

Each R⁴ and R⁵ is independently selected from the group consisting of H, halo, C₁-C₆-alkyl, and C₃-C₇-cycloalkyl. In some embodiments, any two R⁴ and R⁵ bound to the same carbon atom, together with the carbon atom to which they are bound, represent a C₃-C₅-cycloalkyl optionally substituted by 1 to 3 R^{A}, or they represent a C₂-C₆-alkenyl. Illustrating these embodiments of the unit —(CR⁴R⁵)ₘ— are the following substructures:

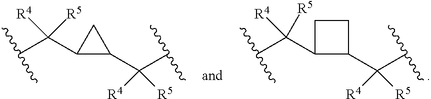

In still other embodiments, any two of R⁴ and R³ not bound to the same carbon atom, together with the respective carbon atoms to which they are bound, represent a C₃-C₇-cycloalkyl optionally substituted by 1 to 3 R^{A}. Illustrating these embodiments of the unit —(CR⁴R⁵)ₘ— are the following substructures:

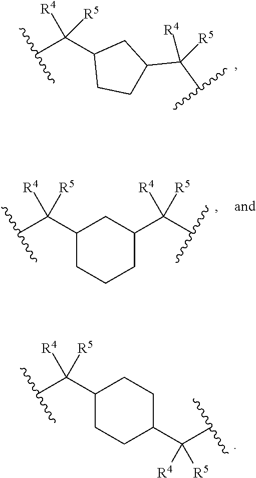

Each instance of R^{A} is independently selected from the group consisting of H, halo, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₁-C₆-alkyl)COOH, —C(O)(C₁-C₆-alkyl)C(O)(C₁-C₆-alkoxy), —C(O)N(H or C₁-C₆-alkyl)₂, —C(O)(C₃-C₁₄-cycloalkyl), —C₃-C₁₄-cycloalkyl, —(C₁-C₆-alkyl)(C₃-C₁₄-cycloalkyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S) that is optionally substituted with C₁-C₆-alkyl.

In various embodiments:
Y¹ and Y² are independently selected from —O— and —CR₄R₅—;
each R¹ and R³ is independently selected from the group consisting of H, halo, C₁-C₆-alkyl, C₂-C₆-alkenyl, C₂-C₆-alkynyl, C₁-C₆-alkoxyl, cyano, and C₁-C₆-haloalkyl, wherein any alkyl, alkenyl, alkynyl or alkoxyl is optionally substituted by 1 to 4 R^{A};
R² is selected from the group consisting of —C(O)OR, —C(O)NHR, C₃-C₆-cycloalkenyl, and 3- to 10-membered heterocyclyl, wherein any alkyl, cycloalkenyl, or heterocyclyl is optionally substituted by 1 to 4 R^{A};
R is selected from the group consisting of H, C₁-C₆-alkyl optionally substituted with —((C₁-C₆-alkyl)OC(O)OC₁-C₆-alkyl) or 3- to 10-membered heterocyclyl, and —(C₁-C₆-alkyl)(C₆-C₁₀-aryl);
each R⁴ and R⁵ is independently selected from the group consisting of H, halo, C₁-C₆-alkyl, and C₃-C₇-cycloalkyl, wherein
optionally any two R⁴ and R⁵ bound to the same carbon atom, together with the carbon atom to which they are bound, represent a C₃-C₅-cycloalkyl optionally substituted by 1 to 3 R^{A}; and
optionally any two of R⁴ and R⁵ not bound to the same carbon atom, together with the respective carbon atoms to which they are bound, represent a C₃-C₇-cycloalkyl optionally substituted by 1 to 3 R^{A}; and
each R^{A} is independently selected from the group consisting of H, halo, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₁-C₆-alkyl)COOH, —C(O)(C₃-C₁₄-cycloalkyl), —C₃-C₁₄-cycloalkyl, —(C₁-C₆-alkyl)(C₃-C₁₄-cycloalkyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

In some embodiments, optionally in combination with any other embodiment described herein, ring B is the same as ring C. In other embodiments, optionally in combination with any other embodiment described herein, ring B is different from ring C.

In illustrative embodiments where ring B is different from ring C, ring B conforms to formula (a), wherein ring A is a 5- or 6-membered monocyclic heteroaryl comprising 1 to 3 heteroatoms selected from O, S, and N. Examples of the ring A monocyclic heteroaryl are selected from the group consisting of pyridinyl, pyridazinyl, pyrazinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl. In some embodiments, the ring A monocyclic heteroaryl is pyridinyl, pyridazinyl, pyrazinyl, or pyrimidinyl. Within ring B, in these embodiments, ring A is optionally substituted by 1 to 4 R^{A}. For example, ring A is substituted by one R^{A} that is a 5- to 10-membered heteroaryl (wherein 1-4 heteroaryl members are independently selected from N, O, and S), such as tetrazolyl, imidazolyl, or triazolyl.

Further in combination with these embodiments, ring C also is of formula (a), wherein ring A is an 8- to 10-membered bicyclic heteroaryl comprising 1 to 6 heteroatoms selected from O, S, and N, optionally substituted by 1 to 4 R^{A}. Non-limiting examples of bicyclic heteroaryl rings include indolizinyl, benzothienyl, quinazolinyl, purinyl, indolyl, quinolinyl, tetrazolo[1,5-b]pyridazinyl, [1,2,3]triazolo[1,5-b]pyridazinyl, [1,2,4]triazolo[1,5-a]pyrimidinyl, [1,2,4]triazolo[4,3-a]pyrimidinyl, and imidazo[1,2-a]pyrimidinyl.

Additional embodiments of the disclosure provide a formula (I) compound wherein ring B and ring C are the same and each is of formula (a). In these embodiments, Ring A is a 5- or 6-membered monocyclic heteroaryl comprising 1 to 3 heteroatoms selected from O, S, and N, and ring A is optionally substituted by 1 to 4 R^{A}. Examples of the monocyclic heteroaryl ring include but are not limited to pyridinyl, pyridazinyl, pyrazinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, and furanyl.

In other embodiments, ring B and ring C are the same and are of formula (a). In these embodiments, ring A is an 8- to 10-membered bicyclic heteroaryl.

The present disclosure also provides, in other embodiments, formula (I) compounds wherein B is Het that is optionally substituted by 1 to 4 R^{A}, and ring C is of formula (a). Illustrative examples of Het include indolizinyl, benzothienyl, quinazolinyl, purinyl, indolyl, quinolinyl, tetrazolo[1,5-b]pyridazinyl, [1,2,3]triazolo[1,5-b]pyridazinyl, [1,2,4]triazolo[1,5-a]pyrimidinyl, [1,2,4]triazolo[4,3-a]pyrimidinyl and imidazo[1,2-a]pyrimidinyl. In some embodiments, Het is benzothienyl optionally substituted by 1 to 4 R^{A} selected from the group consisting of halo, C₁-C₆-alkoxy, —C(O)(C₁-C₆-alkyl)COOH. For example, in some embodiments, Het is the following group:

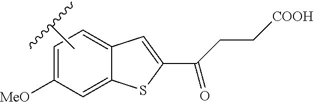

According to some embodiments, optionally in combination with any other embodiment described herein, X is —C(R³)═C(R³)— and W is —C(R³)═.

In various embodiments, each instance of R³ is independently selected from the group consisting of H, halo, and C₁-C₆-alkoxyl.

In still further embodiments, R² is —C(O)OR. For instance, R is H or C₁-C₆-alkyl, such as methyl or ethyl.

In various embodiments, x and y are 0 and 0, 0 and 1, 1 and 0, or 1 and 1, respectively. For example, in some embodiments each of x and y is 1, and each of Y¹ and Y² is —O— or each of Y¹ and Y² is —CR₄R₅—. In an embodiment, each of x and y is 1, each of Y¹ and Y² is —O—, and m is 4. In another embodiment, each of Y¹ and Y² is —CR₄R₅—, each of x and y is 1, m is 1. All these combinations are contemplated.

In various embodiments, optionally in combination with any other embodiment described herein, each R¹ is independently selected from H and halo. For example, in embodiments where ring B or ring C is of formula (a), R¹ is H or halo. In embodiments wherein ring B or ring C is of formula (b), n can be 0, 1, or 2, and in each instance R¹ is H or halo.

Still further embodiments of the present disclosure are compounds of formula (I) wherein:
ring B is of formula (a), wherein ring A is a 6-membered monocyclic heteroaryl comprising 1 to 3 heteroatoms selected from O, S, and N, and that is substituted by a 5- to 10-membered heteroaryl (wherein 1-4 heteroaryl members are independently selected from N, O, and S);
ring C is of formula (a), wherein ring A is an 8- to 10-membered bicyclic heteroaryl;
X is —C(R³)═C(R³)— and W is —C(R³)═, wherein each R³ is independently selected from H, halo, and C₁-C₆-alkoxyl;
R¹ is H;
R² is —C(O)OR and R is H or C₁-C₆-alkyl;
each R⁴ and R⁵ is H;
each of x and y is 1; and
each of Y¹ and Y² is —O— and m is 4, or each of Y¹ and Y² is —CH₂— and m is 1.

In additional embodiments, the present disclosure provides a compound of formula (I) wherein:
each of rings B and C is of formula (a), wherein each ring A is a 6-membered monocyclic heteroaryl comprising 1 to 3 heteroatoms selected from O, S, and N, and that is substituted by one R^{A} that is a 5- to 10-membered heteroaryl (wherein 1-4 heteroaryl members are independently selected from N, O, and S);
X is —C(R³)═C(R³)— and W is —C(R³)═, wherein each R³ is independently selected from H and halo;
R¹ is H;
R² is —C(O)OR and R is H;
each of x and y is 1;
m is 0 or 1;
Y¹ is —CR₄R₅— or —(CH₂)_{L1}—N(R^{L})—; and
Y² is —O— or —CR₄R₅—.

For example, in illustrative embodiments optionally in combination with any other embodiment described herein, each ring A is pyridazinyl substituted by one R^{A} that is imidazolyl.

In further embodiments, the present disclosure provides specific examples of formula (I) compounds, and their pharmaceutically acceptable salts, as set forth in Table 1 below. The compounds are presented with physico-chemical characterizing data.

TABLE 1
Examples of Formula (I) Compounds and Selected Analytical Data.

| Com- | | |
|---|---|---|
| pound | Structure | Analytical Data |
| 1 | 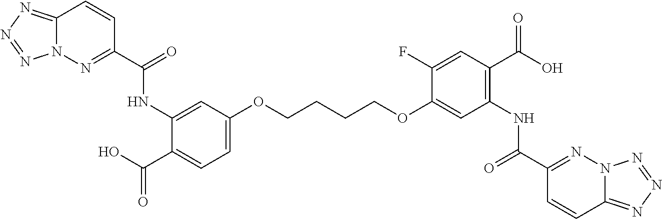 [SMILES: O=C(Nc1cc(OCCCCOc2cc(NC(=O)c3ccc4nnnn4n3)c(C(=O)O)cc2F)ccc1C(=O)O)c1ccc2nnnn2n1] | ¹H NMR (400 MHz, DMSO-d₆) δ 13.71 (s, 2H), 8.97 (d, J = 9.4 Hz, 2H), 8.64 (d, J = 8.0 Hz, 1H), 8.49- 8.27 (m, 3H), 8.04 (d, J = 8.7 Hz, 1H), 7.77 (d, J = 12.0 Hz, 1H), 6.87 (d, J = 8.9 Hz, 1H), 4.37-4.15 (m, 4H), 2.14-1.90 (m, 4H). MS-ESI: m/z 673.2 observed [M + H]⁺ |
| 2 | 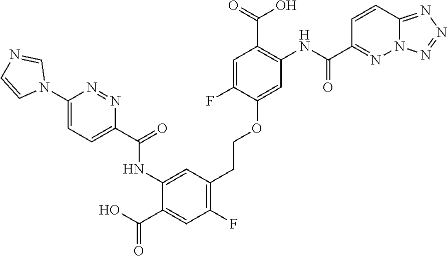 [SMILES: O=C(Nc1cc(CCOc2cc(NC(=O)c3ccc4nnnn4n3)c(C(=O)O)cc2F)c(F)cc1C(=O)O)c1ccc(-n2ccnc2)nn1] | ¹H NMR (400 MHz, DMSO-d₆) δ 16.69 (s, 1H), 15.77 (s, 1H), 8.95 (d, J = 9.6 Hz, 1H), 8.85 (d, J = 7.2 Hz, 1H), 8.80 (s, 1H), 8.64 (d, J = 8.0 Hz, 1H), 8.47 (d, J = 8.8 Hz. 1H), 8.41 (d, J = 9.2 Hz, 1H), 8.36 (d, J = 9.6 Hz, 1H), 8.21 (s, 1H), 7.77 (d, J = 11.6 Hz, 1H), 7.27 (s, 1H), 4.35 (t, J = 6.8 Hz, 2H), 3.21 (t, J = 6.0 Hz, 2H). MS-ESI: m/z 672.14 observed [M + H]⁺ |
| 3 | 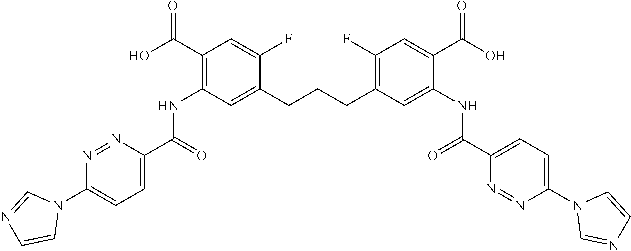 [SMILES: O=C(Nc1cc(CCCc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 8.77 (s, 2H), 8.74 (d, J = 7.3 Hz, 2H), 8.43 (d, J = 9.1 Hz, 2H), 8.38 (d, J = 9.1 Hz, 2H), 8.18 (t. J = 1.4 Hz, 2H), 7.69 (d, J = 10.9 Hz, 2H), 7.27-7.21 (m, 2H), 2.74 (t, J = 7.7 Hz, 5H), 1.98- 1.91 (m, 2H). MS-ESI: m/z 695.18 observed [M + H]⁺ |
| 4 | 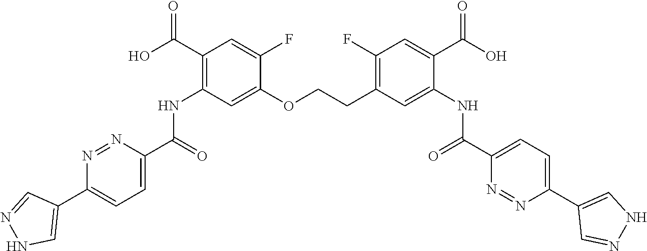 [SMILES: O=C(Nc1cc(CCOc2cc(NC(=O)c3ccc(-c4cn[nH]c4)nn3)c(C(=O)O)cc2F)c(F)cc1C(=O)O)c1ccc(-c2cn[nH]c2)nn1] | ¹H NMR (500 MHz, DMSO-d₆) δ 9.15 (t, J = 6.5 Hz, 1H), 8.82 (d, J = 7.0 Hz, 1H), 8.70 (dd, J = 8.2, 4.2 Hz, 1H), 8.57 (d, J = 3.4 Hz, 1H), 8.36-8.05 (m, 6H), 7.73 (d, J = 11.6 Hz, 2H), 5.50-5.38 (m, 2H), 4.31 (t, J = 7.0 Hz, 2H). MS-ESI: m/z 697.16 observed [M + H]⁺ |
| 5 | 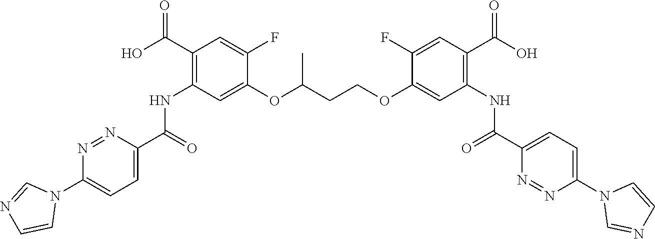 [SMILES: CC(CCOc1cc(NC(=O)c2ccc(-n3ccnc3)nn2)c(C(=O)O)cc1F)Oc1cc(NC(=O)c2ccc(-n3ccnc3)nn2)c(C(=O)O)cc1F] | ¹H NMR (400 MHz, DMSO-d₆) δ 16.08 (s, 1H), 16.05 (s, 1H), 8.78 (s, 2H), 8.73- 8.68 (m, 2H), 8.48- 8.45 (m, 2H), 8.40 (d, J = 8.8 Hz, 2H), 8.19 (s, 2H), 7.75 (dd, J = 12.4, 4.4 Hz, 2H), 7.25 (s, 2H), 4.80-4.61 (m, 1H), 4.28-4.26 (m, 2H), 2.34-2.28 (m, 2H), 1.45-1.43 (m, 4H). MS-ESI: m/z 741.2 observed [M + H]⁺ |
| 6 | 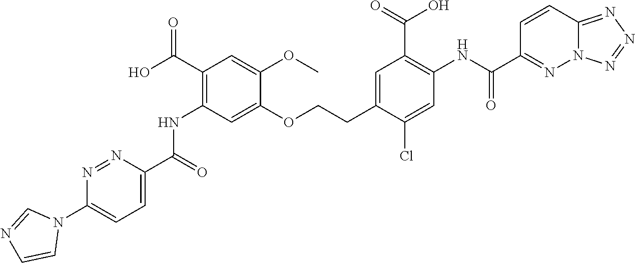 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1Cl] | ¹H NMR (500 MHz, DMSO-d₆) δ 15.59 (s, 1H), 8.94 (d, J = 9.6 Hz, 1H), 8.81 (s, 1H), 8.76 (s, 1H), 8.59 (s, 1H), 8.44-8.34 (m, 3H), 8.17 (s, 2H), 7.67 (s, 1H), 7.24 (s, 1H), 4.21 (t. J = 7.2 Hz, 2H), 3.23 (t, J = 7.2 Hz, 2H). MS-ESI: m/z 700.2 observed [M + H]⁺ |
| 7 | 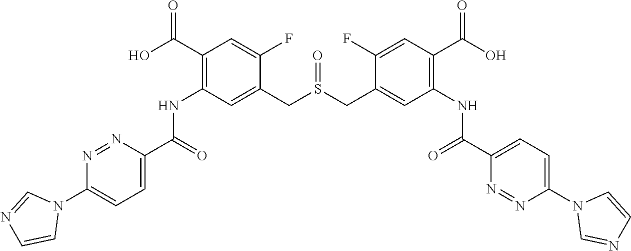 [SMILES: O=C(Nc1cc(CS(=O)Cc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 15.72 (s, 2H), 8.87-8.82 (m, 2H), 8.77 (s, 2H), 8.48-8.36 (m, 4H), 8.19 (s, 2H), 7.78 (d, J = 12.8 Hz, 2H), 7.25 (s, 2H), 4.38 (d, J = 13.2 Hz, 2H), 4.18 (s, 2H). MS-ESI: m/z 729.2 observed [M + H]⁺ |
| 8 | 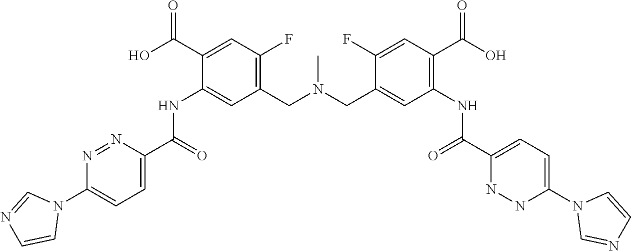 | 1H NMR (400 MHz, DMSO-d₆) δ 15.70 (s, 2H), 8.85 (d, J = 7.0 Hz, 2H), 8.77 (s, 2H), 8.45-8.30 (m, 4H), 8.18 (s, 2H), 7.71 (d, J = 10.8 Hz, 2H), 7.25 (s, 2H), 3.66 (s, 4H), 2.20 (s, 3H). MS-ESI: m/z 710.47 observed [M + H]⁺ |
| 9 | 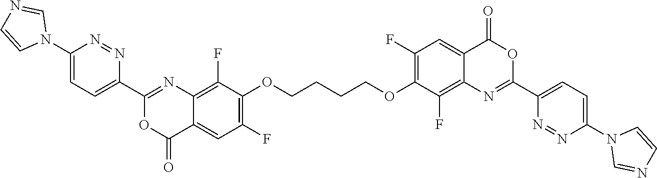 [SMILES: O=c1oc(-c2ccc(-n3ccnc3)nn2)nc2c(F)c(OCCCCOc3c(F)cc4c(=O)oc(-c5ccc(-n6ccnc6)nn5)nc4c3F)c(F)cc12] | ¹H NMR (400 MHz, DMSO-d₆) δ 8.79 (s, 2H), 8.64 (d, J = 9.2 Hz, 2H), 8.42 (d, J = 9.2 Hz, 2H), 8.19 (s, 2H), 7.98 (dd, J = 1.2, 10.4 Hz, 2H), 7.26 (s, 2H), 4.52 (br s, 4H), 1.99 (br s, 4H). MS-ESI: m/z 741.3 observed [M + H]⁺ |
| 10 | 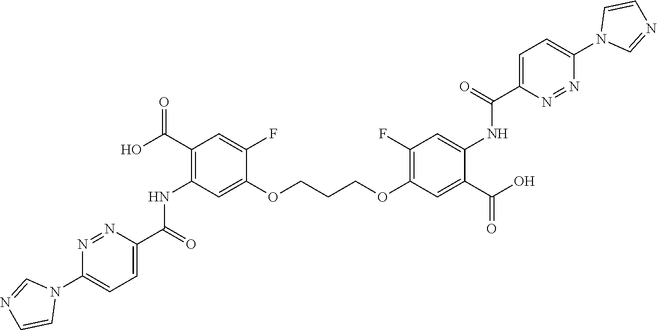 [SMILES: O=C(Nc1cc(OCCCOc2cc(C(=O)O)c(NC(=O)c3ccc(-n4ccnc4)nn3)cc2F)c(F)cc1C(=O)O)c1ccc(-n2ccnc2)nn1] | ¹H NMR (500 MHz, DMSO-d₆) δ 8.78 (s, 2H), 8.71 (d, J = 8.2 Hz, 1H), 8.62 (d, J = 14.1 Hz, 1H), 8.51- 8.37 (m, 4H), 8.19 (s, 2H), 7.81 (dd, J = 50.9, 11.2 Hz, 2H), 7.25 (s, 2H), 4.28 (d, J = 21.7 Hz, 4H), 2.36 (s, 2H). MS-ESI: m/z 727.2 observed [M + H]⁺ |
| 11 | 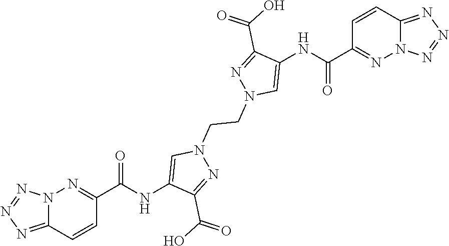 [SMILES: O=C(Nc1cn(CCn2cc(NC(=O)c3ccc4nnnn4n3)c(C(=O)O)n2)nc1C(=O)O)c1ccc2nnnn2n1] | ¹H NMR (500 MHz, DMSO-d₆) δ 8.67 8.46 (m, 2H), 8.45- 8.28 (m, 2H), 8.05- 7.87 (m, 2H), 4.42- 4.06 (m, 4H). MS-ESI: m/z 574.96 observed [M + H]⁺ |
| 12 | 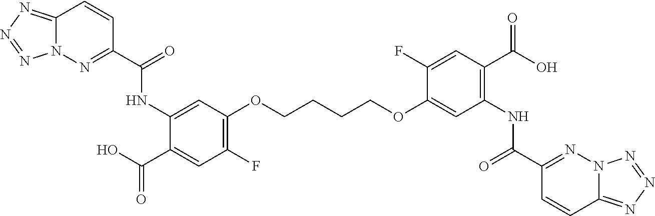 [SMILES: O=C(Nc1cc(OCCCCOc2cc(NC(=O)c3ccc4nnnn4n3)c(C(=O)O)cc2F)c(F)cc1C(=O)O)c1ccc2nnnn2n1] | ¹H NMR (400 MHz, DMSO-d₆) δ 13.89 (s, 2H), 8.98 (d, J = 9.4 Hz, 2H), 8.66 (d, J = 7.8 Hz, 2H), 8.36 (d, J = 9.4 Hz, 2H), 7.80 (d, J = 11.8 Hz, 2H), 4.30 (s, 4H), 2.05 (d, J = 17.6 Hz, 4H). MS-ESI: m/z 691.2 observed [M + H]⁺ |
| 13 | 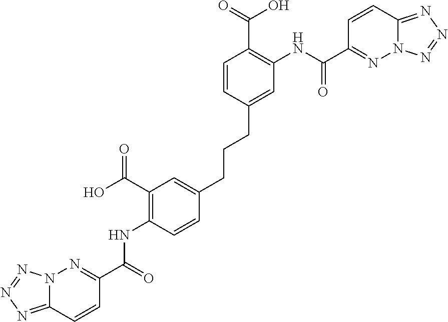 [SMILES: O=C(Nc1cc(CCCc2ccc(NC(=O)c3ccc4nnnn4n3)c(C(=O)O)c2)ccc1C(=O)O)c1ccc2nnnn2n1] | ¹H NMR (400 MHz, DMSO-d₆) δ 8.92 (dd, J = 9.5, 3.9 Hz, 2H), 8.60 (dd, J = 5.2, 3.4 Hz, 2H), 8.41- 8.28 (m, 2H), 8.03-7.89 (m, 2H), 7.27 (d, J = 8.4 Hz, 1H), 6.97 (d, J = 8.0 Hz, 1H), 2.72-2.62 (m, 4H), 1.99-1.88 (m, 2H). MS-ESI: m/z 609.25 observed [M + H]⁺ |
| 14 | 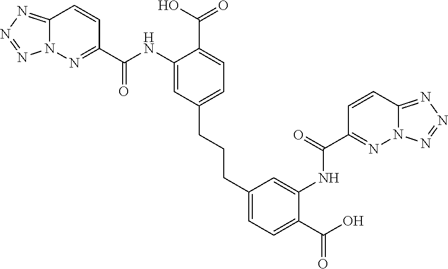 [SMILES: O=C(Nc1cc(CCCc2ccc(C(=O)O)c(NC(=O)c3ccc4nnnn4n3)c2)ccc1C(=O)O)c1ccc2nnnn2n1] | ¹H NMR (400 MHz, DMSO-d₆) δ 8.94 (d., J = 9.2 Hz, 2H), 8.62 (s, 2H), 8.36 (d, J = 9.42 Hz, 2H), 8.01 (d, J = 8.0 Hz, 2H), 7.00 (d, J = 8.0 Hz, 2H), 2.71-2.69 (m, 4H), 1.99-1.97 (m, 2H). MS-ESI: m/z 609.22 observed [M + H]⁺ |
| 15 | 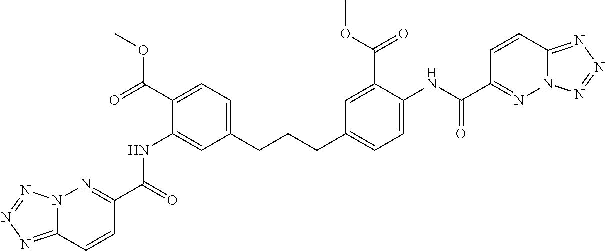 [SMILES: COC(=O)c1ccc(CCCc2ccc(NC(=O)c3ccc4nnnn4n3)c(C(=O)OC)c2)cc1NC(=O)c1ccc2nnnn2n1] | ¹H NMR (400 MHz, DMSO-d₆) δ 12.76 (s, 1H), 12.65 (s, 1H), 9.04 (dd, J = 6.8 Hz, 9.6 Hz, 2H), 8.65- 8.62 (m, 2H), 8.40 (dd, J = 6.0 Hz, 9.6 Hz, 2H), 8.05 (d, J = 8.4 Hz, 1H), 7.96 (d, J = 2.0 Hz, 1H), 7.63 (d, J = 2.0 Hz, 1H), 7.23 (d. J = 8.0 Hz, 1H), 3.98 (s, 6H), 2.81-2.69 (m, 4H), 2.04-2.01 (m, 2H). MS-ESI: m/z 637.1 observed [M + H]⁺ |
| 16 | 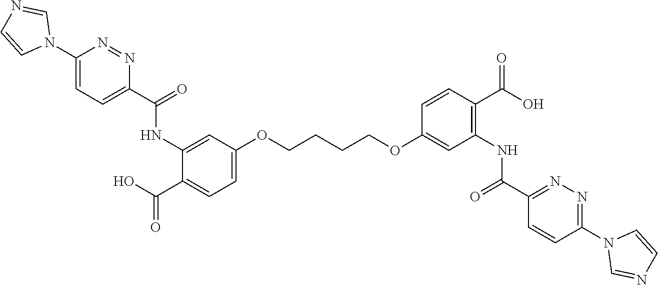 [SMILES: O=C(Nc1cc(OCCCCOc2ccc(C(=O)O)c(NC(=O)c3ccc(-n4ccnc4)nn3)c2)ccc1C(=O)O)c1ccc(-n2ccnc2)nn1] | ¹H NMR (400 MHz, DMSO-d₆) δ 16.13 (s, 2H), 8.77 (s, 2H), 8.52-8.33 (m, 4H), 8.23-8.13 (m, 2H), 7.98 (d, J = 8.6 Hz, 2H), 7.25 (s, 2H), 6.73- 6.55 (m, 4H), 4.16-4.02 (m, 4H), 2.01-1.85 (m, 4H). MS-ESI: m/z 705.45 observed [M + H]⁺ |
| 17 | 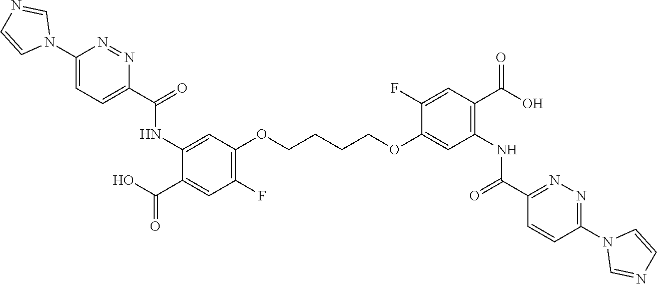 [SMILES: O=C(Nc1cc(OCCCCOc2cc(NC(=O)c3ccc(-n4ccnc4)nn3)c(C(=O)O)cc2F)c(F)cc1C(=O)O)c1ccc(-n2ccnc2)nn1] | MS-ESI: m/z 741.31 observed [M + H]⁺ |
| 18 | 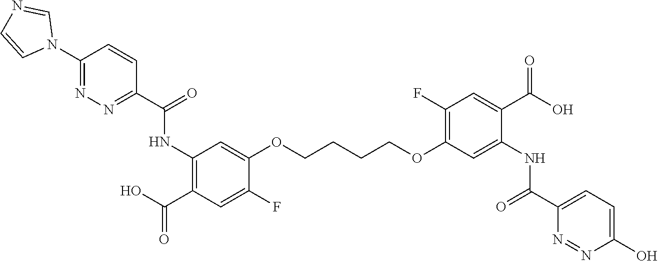 [SMILES: O=C(Nc1cc(OCCCCOc2cc(NC(=O)c3ccc(-n4ccnc4)nn3)c(C(=O)O)cc2F)c(F)cc1C(=O)O)c1ccc(O)nn1] | ¹H NMR (400 MHz, DMSO-d₆) δ 13.67 (s, 1H), 13.28 (s, 1H), 12.64 (s, 1H), 8.83 (s. 1H), 8.76 (d, J = 7.9 Hz, 1H), 8.64 (d, J = 8.2 Hz, 1H), 8.54- 8.42 (m, 2H), 8.22 (s, 1H), 7.93 (d, J = 9.8 Hz, 1H), 7.77 (dd, J = 20.2, 11.9 Hz, 2H), 7.29 (s, 1H), 7.06- 6.98 (m, 1H), 4.38- 4.20 (m, 4H), 2.11- 1.93 (m, 4H). MS-ESI: m/z 691.29 observed [M + H]⁺ |
| 19 | 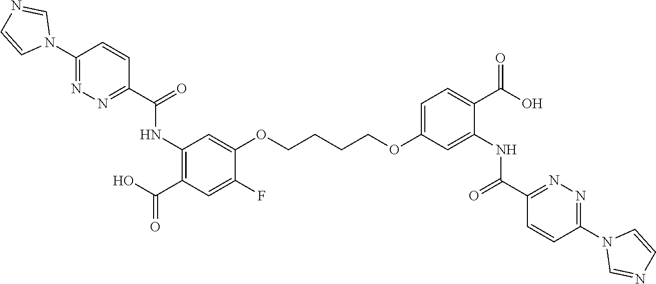 [SMILES: O=C(Nc1cc(OCCCCOc2cc(NC(=O)c3ccc(-n4ccnc4)nn3)c(C(=O)O)cc2F)ccc1C(=O)O)c1ccc(-n2ccnc2)nn1] | ¹H NMR (400 MHz, DMSO-d₆) δ 14.91 (s, 1H), 13.94 (s, 1H), 8.78 (d, J = 5.6, 2H), 8.47 (d, J = 8.4, 1H), 8.45-8.41 (m, 5H), 8.17 (d, J = 6.0, 2H), 8.03-8.01 (m, 1H), 7.77-7.75 (m, 1H), 7.25 (d, J = 4.4, 1H), 6.81 (d, J = 9.6, 1H), 4.23-4.19 (m, 4H), 2.0-1.98 (m, 4H). MS-ESI: m/z 723.1 observed [M + H]⁺ |
| 20 | 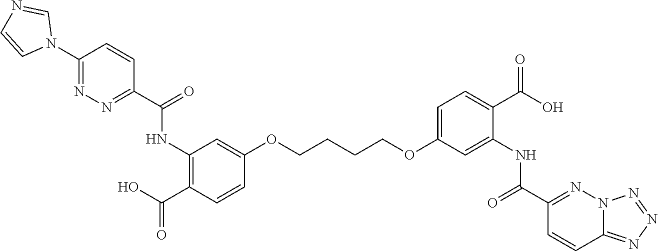 [SMILES: O=C(Nc1cc(OCCCCOc2ccc(C(=O)O)c(NC(=O)c3ccc4nnnn4n3)c2)ccc1C(=O)O)c1ccc(-n2ccnc2)nn1] | ¹H NMR (400 MHz, DMSO-d₆) δ 16.23 (s, 1H), 8.95-8.88 (m, 1H), 8.80-8.75 (m, 1H), 8.48-8.32 (m, 6H), 8.21-8.17 (m, 1H), 7.97 (dd, J = 8.7, 3.0 Hz, 2H), 7.25 (d, J = 1.6 Hz, 1H), 6.66 (ddd, J = 15.5, 8.6, 2.6 Hz, 2H), 4.14-4.05 (m, 4H), 2.01-1.88 (m, 4H). MS-ESI: m/z 680.20 observed [M + H]⁺ |
| 21 | 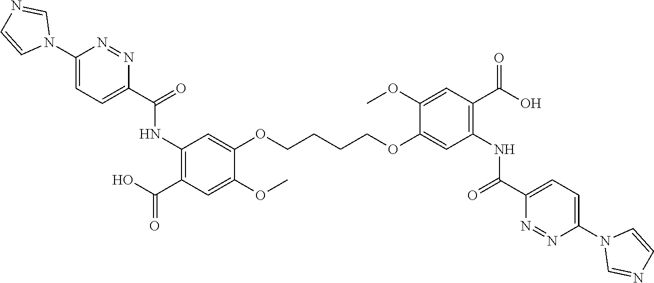 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1OC] | ¹H NMR (400 MHz, DMSO-d₆) δ 8.77 (s, 2H), 8.61 (s, 2H), 8.47 (d, J = 2.4 Hz, 2H), 8.47 (d, J = 8.4 Hz, 2H), 8.47 (d, J = 8.4 Hz, 2H), 8.40 (d, J = 9.2 Hz, 2H), 8.18 (s, 2H), 7.56 (s, 2H), 7.25 (s, 2H), 4.19-4.17 (m, 4H), 3.77 (s, 6H), 2.00-1.99 (m, 4H). MS-ESI: m/z 765.5 observed [M + H]⁺ |
| 22 | 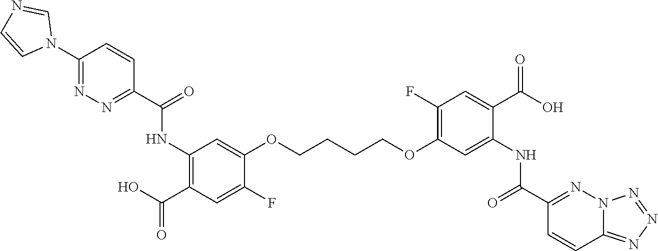 [SMILES: O=C(Nc1cc(OCCCCOc2cc(NC(=O)c3ccc4nnnn4n3)c(C(=O)O)cc2F)c(F)cc1C(=O)O)c1ccc(-n2ccnc2)nn1] | ¹H NMR (400 MHz, DMSO-d₆) δ 16.09 (s, 2H), 8.92 (d, J = 9.5 Hz, 1H), 8.77 (s, 1H), 8.65 (dd, J = 30.5, 8.1 Hz, 2H), 8.49- 8.30 (m, 3H), 8.18 (s, 1H), 7.74 (d, J = 12.8 Hz, 2H), 7.25 (s, 1H), 4.32-4.06 (m, 4H), 2.12-1.90 (m, 4H). MS-ESI: m/z 716.3 observed [M + H]⁺ |
| 23 | 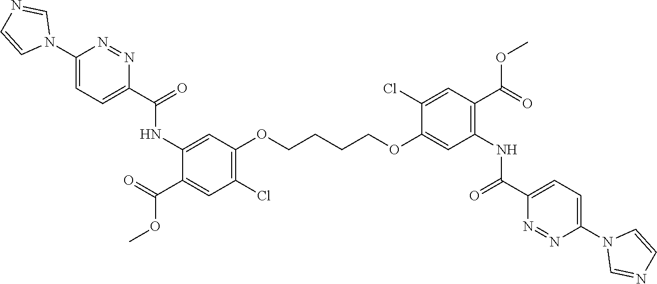 [SMILES: COC(=O)c1cc(Cl)c(OCCCCOc2cc(NC(=O)c3ccc(-n4ccnc4)nn3)c(C(=O)OC)cc2Cl)cc1NC(=O)c1ccc(-n2ccnc2)nn1] | ¹H NMR (400 MHz, DMSO-d₆) δ 12.87 (s, 2H), 8.69-8.67 (m, 4H), 8.45-8.37 (m, 4H), 8.11 (s, 2H), 7.99 (s, 2H), 7.24 (d, J = 0.8 Hz, 2H), 4.37-4.35 (m, 4H), 3.93 (s, 6H), 2.11-2.09 (m, 4H). MS-ESI: m/z 801.1 observed [M + H]⁺ |
| 24 | 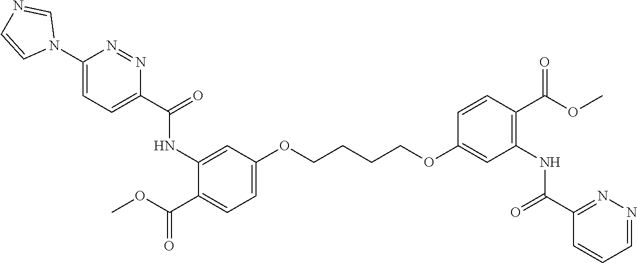 [SMILES: COC(=O)c1ccc(OCCCCOc2ccc(C(=O)OC)c(NC(=O)c3ccc(-n4ccnc4)nn3)c2)cc1NC(=O)c1cccnn1] | ¹H NMR (400 MHz, DMSO-d₆) δ 13.17 (s, 2H), 9.47 (dd, J = 5.0, 1.8 Hz, 1H), 8.89 (d, J = 2.3 Hz, 1H), 8.50-8.45 (m, 3H), 8.32 (dd, J = 8.5, 1.8 Hz, 2H), 8.25 (s, 1H), 8.03-7.96 (m, 3H), 7.31 (s, 1H), 6.86 (d, J = 9.0 Hz, 2H), 4.23-4.21 (m, 4H), 3.88 (s, 6H), 2.00-1.98 (s, 4H). MS-ESI: m/z 667.35 observed [M + H]⁺ |
| 25 | 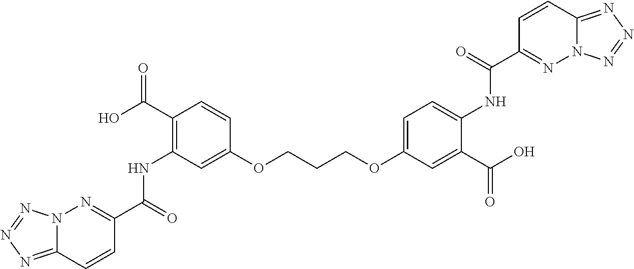 [SMILES: O=C(Nc1cc(OCCCOc2ccc(NC(=O)c3ccc4nnnn4n3)c(C(=O)O)c2)ccc1C(=O)O)c1ccc2nnnn2n1] | ¹H NMR (499 MHz, DMSO-d₆) δ 8.91 (dd, J = 9.4, 5.2 Hz, 2H), 8.62 (d, J = 8.9 Hz, 1H), 8.40-8.30 (m, 3H), 7.98 (d, J = 8.6 Hz, 1H), 7.67 (d, J = 3.2 Hz, 1H), 7.00 (dd, J = 9.0, 3.2 Hz, 1H), 6.69 (dd, J = 8.6, 2.6 Hz, 1H), 4.25- 4.13 (m, 4H), 2.23 (q, J = 6.3 Hz, 2H). MS-ESI: m/z 640.64 observed [M + H]⁺ |
| 26 | 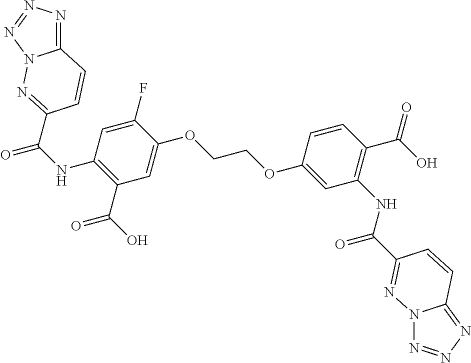 [SMILES: O=C(Nc1cc(OCCOc2cc(C(=O)O)c(NC(=O)c3ccc4nnnn4n3)cc2F)ccc1C(=O)O)c1ccc2nnnn2n1] | ¹H NMR (400 MHz, DMSO-d₆) δ 9.15 (d, J = 9.6 Hz, 2H), 8.92- 8.34 (m, 3H), 8.68 (d, J = 2.4 Hz, 1H), 8.68 (d, J = 2.4 Hz, 1H), 8.55 (d, J = 9.2 Hz, 1H), 8.37 (d, J = 8.8 Hz, 1H), 7.22 (dd, J = 2.4, 8.8 Hz, 1H), 4.24 (br, s, 4H). MS-ESI: m/z 645.14 observed [M + H]⁺ |
| 27 | 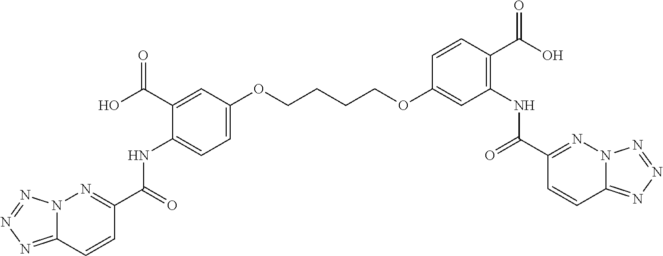 [SMILES: O=C(Nc1cc(OCCCCOc2ccc(NC(=O)c3ccc4nnnn4n3)c(C(=O)O)c2)ccc1C(=O)O)c1ccc2nnnn2n1] | MS-ESI: m/z 655.1 observed [M + H]⁺ |
| 28 | 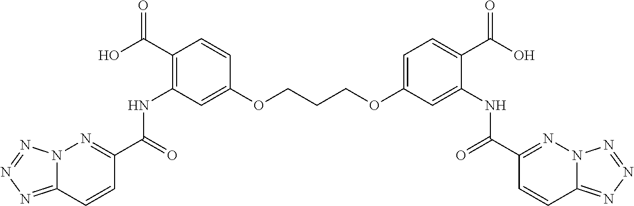 [SMILES: O=C(Nc1cc(OCCCOc2ccc(C(=O)O)c(NC(=O)c3ccc4nnnn4n3)c2)ccc1C(=O)O)c1ccc2nnnn2n1] | MS-ESI: m/z 640.8 observed [M + H]⁺ |
| 29 | 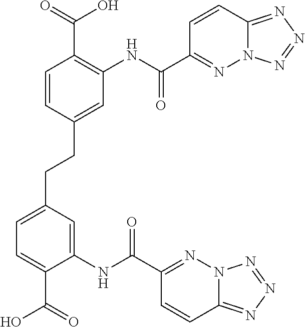 [SMILES: O=C(Nc1cc(CCc2ccc(C(=O)O)c(NC(=O)c3ccc4nnnn4n3)c2)ccc1C(=O)O)c1ccc2nnnn2n1] | MS-ESI: m/z 595.1 observed [M + H]⁺ |
| 30 | 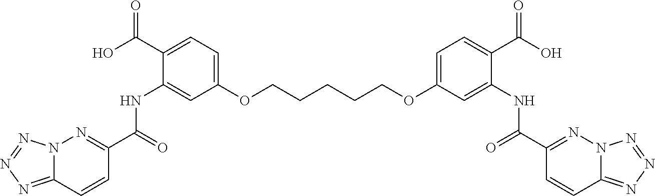 [SMILES: O=C(Nc1cc(OCCCCCOc2ccc(C(=O)O)c(NC(=O)c3ccc4nnnn4n3)c2)ccc1C(=O)O)c1ccc2nnnn2n1] | MS-ESI: m/z 668.9 observed [M + H]⁺ |
| 31 | 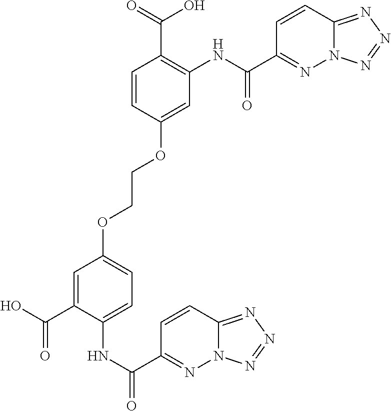 [SMILES: O=C(Nc1cc(OCCOc2ccc(NC(=O)c3ccc4nnnn4n3)c(C(=O)O)c2)ccc1C(=O)O)c1ccc2nnnn2n1] | MS-ESI: m/z 627.2 observed [M + H]⁺ |
| 32 | 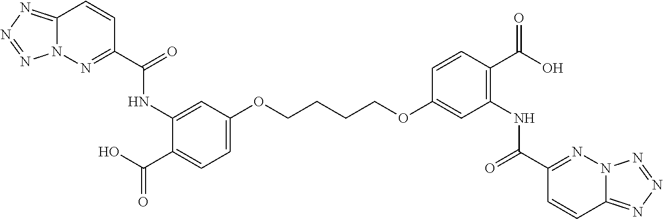 [SMILES: O=C(Nc1cc(OCCCCOc2ccc(C(=O)O)c(NC(=O)c3ccc4nnnn4n3)c2)ccc1C(=O)O)c1ccc2nnnn2n1] | MS-ESI: m/z 655.1 observed [M + H]⁺ |
| 33 | 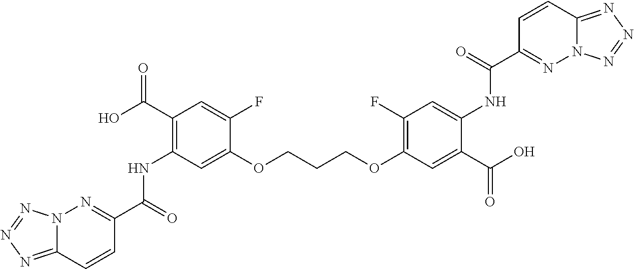 [SMILES: O=C(Nc1cc(OCCCOc2cc(C(=O)O)c(NC(=O)c3ccc4nnnn4n3)cc2F)c(F)cc1C(=O)O)c1ccc2nnnn2n1] | MS-ESI: m/z 677.12 observed [M + H]⁺ |
| 34 | 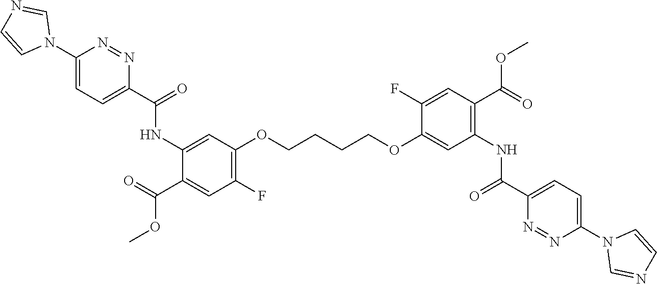 [SMILES: COC(=O)c1cc(F)c(OCCCCOc2cc(NC(=O)c3ccc(-n4ccnc4)nn3)c(C(=O)OC)cc2F)cc1NC(=O)c1ccc(-n2ccnc2)nn1] | MS-ESI: m/z 769.2 observed [M + H]⁺ |
| 35 | 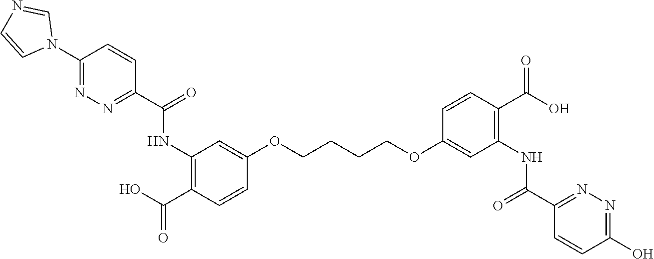 [SMILES: O=C(Nc1cc(OCCCCOc2ccc(C(=O)O)c(NC(=O)c3ccc(-n4ccnc4)nn3)c2)ccc1C(=O)O)c1ccc(O)nn1] | MS-ESI: m/z 655.49 observed [M + H]⁺ |
| 36 | 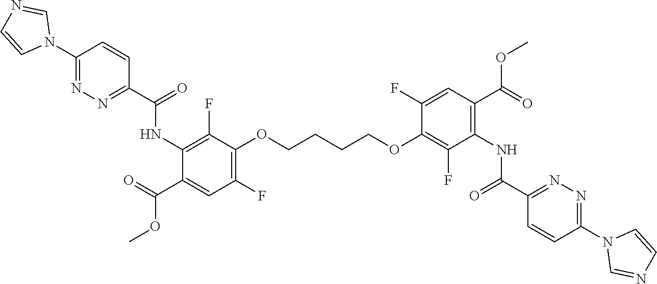 [SMILES: COC(=O)c1cc(F)c(OCCCCOc2c(F)cc(C(=O)OC)c(NC(=O)c3ccc(-n4ccnc4)nn3)c2F)c(F)c1NC(=O)c1ccc(-n2ccnc2)nn1] | ¹H NMR (400 MHz, DMSO-d₆) δ 11.07 (s, 2H), 9.31 (s, 2H), 8.57-8.50 (dd, J = 20, 9.2 Hz, 4H), 8.37 (s, 2H), 7.67-7.64 (m, 2H), 7.55 (s, 2H), 4.36 (s, 4H), 3.74 (s, 6H), 1.92 (s, 4H). MS-ESI: m/z 805.3 observed [M + H]⁺ |
| 37 | 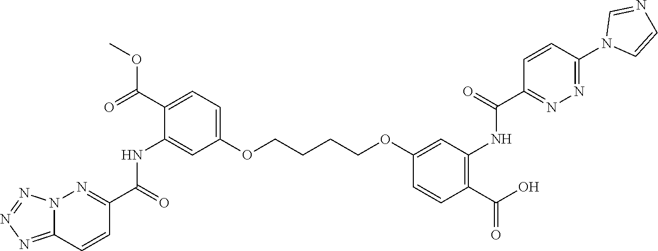 [SMILES: COC(=O)c1ccc(OCCCCOc2ccc(C(=O)O)c(NC(=O)c3ccc(-n4ccnc4)nn3)c2)cc1NC(=O)c1ccc2nnnn2n1] | MS-ESI: m/z 694.1 observed [M + H]⁺ |
| 38 | 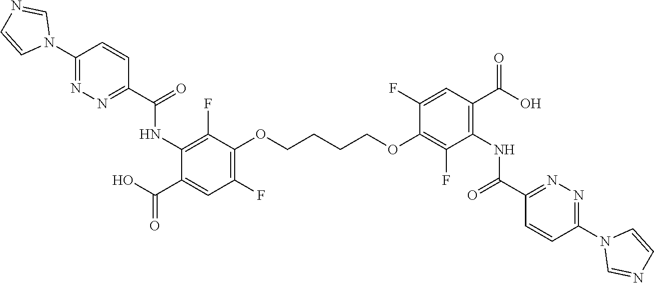 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(-n2ccnc2)nn1] | ¹H NMR (400 MHz, DMSO-d₆) δ 12.00 (s, 2H), 8.79 (s, 2H), 8.48 (d, J = 9.2 Hz, 4H), 8.42 (d, J = 9.2 Hz, 2H), 8.19 (m, 2H), 7.60 (d, J = 11.6 Hz, 2H), 7.26 (s, 2H), 4.30 (s, 4H), 1.92 (s, 4H). MS-ESI: m/z 777.1 observed [M + H]⁺ |
| 39 | 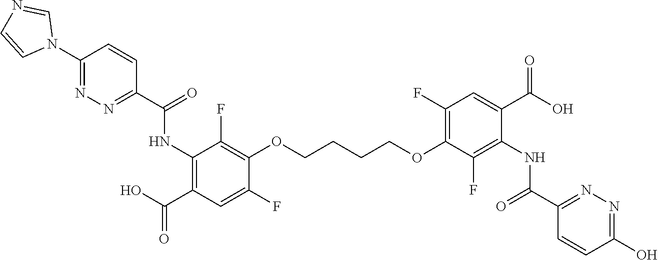 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(O)nn1] | MS-ESI: m/z 727.48 observed [M + H]⁺ |
| 40 | 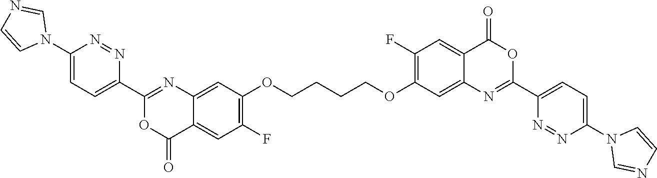 [SMILES: O=c1oc(-c2ccc(-n3ccnc3)nn2)nc2cc(OCCCCOc3cc4nc(-c5ccc(-n6ccnc6)nn5)oc(=O)c4cc3F)c(F)cc12] | ¹H NMR (400 MHz, DMSO-d₆) δ 8.79 (s, 2H), 8.62 (d, J = 9.2 Hz, 2H), 8.48 (d, J = 9.2 Hz, 4H), 8.44 (d, J = 9.2 Hz, 2H), 8.19 (d, J = 1.2 Hz, 2H), 8.00 (d, J = 8.1 Hz, 2H), 7.58 (d, J = 7.6 Hz, 2H), 7.27 (s, 2H), 4.43 (d, J = 4.8 Hz, 4H), 2.05-1.99 (m, 4H). MS-ESI: m/z 705.2 observed [M + H]⁺ |
| 41 | 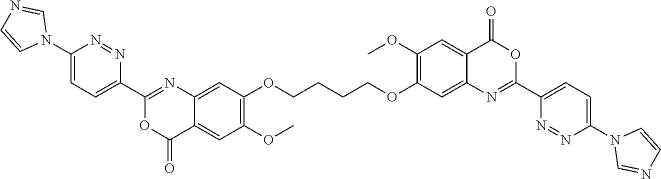 [SMILES: COc1cc2c(=O)oc(-c3ccc(-n4ccnc4)nn3)nc2cc1OCCCCOc1cc2nc(-c3ccc(-n4ccnc4)nn3)oc(=O)c2cc1OC] | ¹H NMR (400 MHz, DMSO-d₆) δ 8.78 (s, 2H), 8.61 (d, J = 9.6 Hz, 2H), 8.43 (d, J = 9.6 Hz, 2H), 8.19 (s, 2H), 7.55 (s, 2H), 7.37 (s, 2H), 7.27 (s, 2H), 4.34-4.36 (m, 4H), 3.95 (s, 6H), 1.99-2.04 (m, 4H). MS-ESI: m/z 729.2 observed [M + H]⁺ |
| 42 | 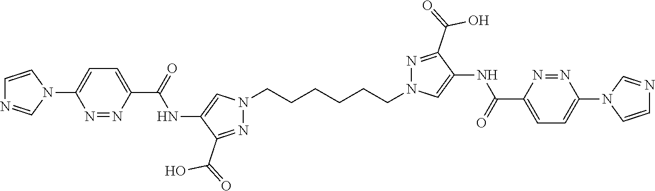 [SMILES: O=C(Nc1cn(CCCCCCn2cc(NC(=O)c3ccc(-n4ccnc4)nn3)c(C(=O)O)n2)nc1C(=O)O)c1ccc(-n2ccnc2)nn1] | MS-ESI: m/z 681.2 observed [M + H]⁺ |
| 43 | 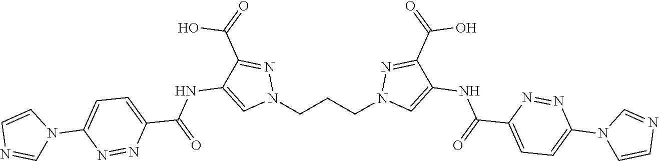 [SMILES: O=C(Nc1cn(CCCn2cc(NC(=O)c3ccc(-n4ccnc4)nn3)c(C(=O)O)n2)nc1C(=O)O)c1ccc(-n2ccnc2)nn1] | MS-ESI: m/z 639.17 observed [M + H]⁺ |
| 44 | 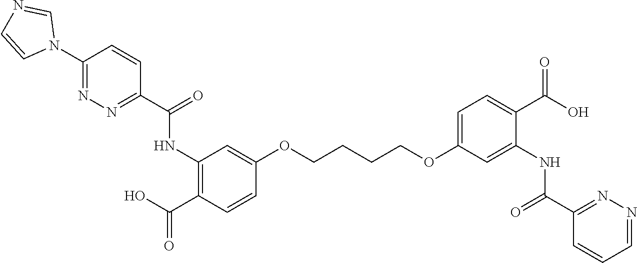 [SMILES: O=C(Nc1cc(OCCCCOc2ccc(C(=O)O)c(NC(=O)c3ccc(-n4ccnc4)nn3)c2)ccc1C(=O)O)c1cccnn1] | MS-ESI: m/z 639.6 observed [M + H]⁺ |
| 45 | 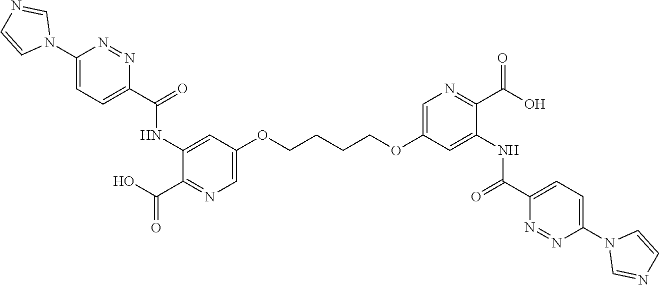 [SMILES: O=C(Nc1cc(OCCCCOc2cnc(C(=O)O)c(NC(=O)c3ccc(-n4ccnc4)nn3)c2)cnc1C(=O)O)c1ccc(-n2ccnc2)nn1] | ¹H NMR (400 MHz, DMSO-d₆) δ 15.52 (s, 2H), 8.82-8.77 (m, 4H), 8.44-8.38 (m, 4H), 8.17 (s, 2H), 7.97 (s, 2H), 7.24 (s, 2H), 4.24-4.23 (m, 4H), 1.98-1.97 (m, 4H). MS-ESI: m/z 707.1 observed [M + H]⁺ |
| 46 | 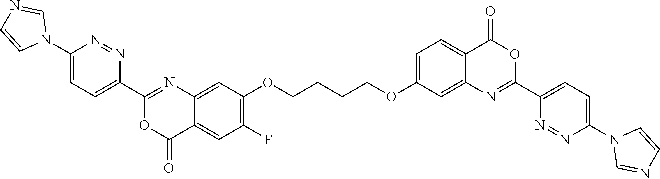 [SMILES: O=c1oc(-c2ccc(-n3ccnc3)nn2)nc2cc(OCCCCOc3cc4nc(-c5ccc(-n6ccnc6)nn5)oc(=O)c4cc3F)ccc12] | ¹H NMR (400 MHz, DMSO-d₆) δ 8.69 (s, 2H), 8.58-8.56 (m, 2H), 8.36-8.33 (m, 2H), 8.13-8.10 (m, 3H), 7.92 (d, J = 12.5 Hz, 1H), 7.55 (d, J = 7.2 Hz, 1H), 7.28- 7.25 (m, 4H), 4.45- 4.44 (m, 2H), 4.38- 4.37 (m, 2H), 2.08- 2.05 (m, 4H), MS-ESI: m/z 687.3 observed [M + H]⁺ |
| 47 | 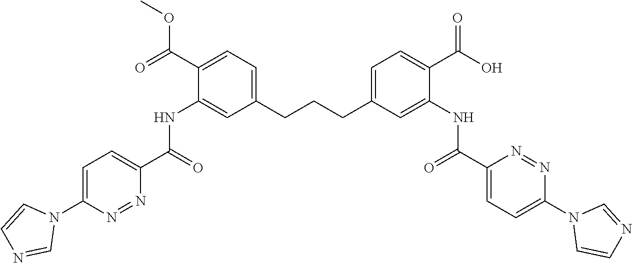 [SMILES: COC(=O)c1ccc(CCCc2ccc(C(=O)O)c(NC(=O)c3ccc(-n4ccnc4)nn3)c2)cc1NC(=O)c1ccc(-n2ccnc2)nn1] | ¹H NMR (400 MHz, DMSO-d₆) δ 9.59- 9.57 (m, 2H), 8.78- 8.76 (m, 2H), 8.51- 8.49 (m, 2H), 8.37- 8.35 (m, 2H), 8.23 (s, 2H), 8.15-8.12 (m, 1H), 8.07-8.05 (m, 1H), 7.65 (s, 2H), 7.18-7.15 (m, 2H), 3.95 (s, 3H), 2.76 (s, 4H), 2.07 (s, 2H), MS-ESI: m/z 673.3 observed [M + H]⁺ |
| 48 | 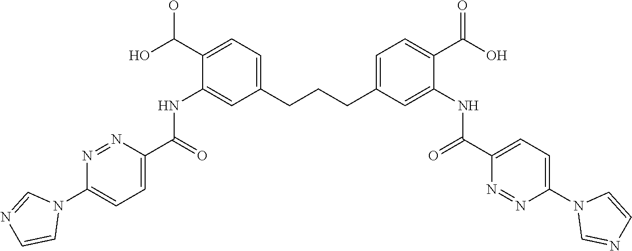 [SMILES: O=C(Nc1cc(CCCc2ccc(C(O)O)c(NC(=O)c3ccc(-n4ccnc4)nn3)c2)ccc1C(=O)O)c1ccc(-n2ccnc2)nn1] | ¹H NMR (500 MHz, DMSO-d₆) δ 10.23 (s, 1H), 8.97 (d, J = 9.4 Hz, 1H), 8.82-8.57 (m, 4H), 8.40-8.29 (m, 2H), 8.04 (d, J = 10.0 Hz, 2H), 7.77 (d, J = 12.0 Hz, 1H), 6.87 (dd, J = 8.8, 2.5 Hz, 1H), 4.38-4.28 (m, 2H), 4.23 (t, J = 5.9 Hz, 2H), 2.03 (dd, J = 16.8, 7.3 Hz, 4H). MS-ESI: m/z 659.2 observed [M + H]⁺ |
| 49 | 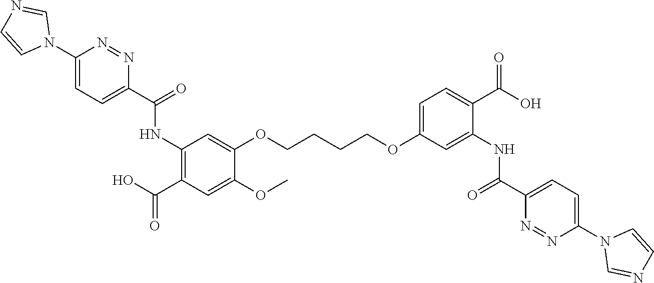 [SMILES: COc1cc(C(=O)O)c(NC(=O)c2ccc(-n3ccnc3)nn2)cc1OCCCCOc1ccc(C(=O)O)c(NC(=O)c2ccc(-n3ccnc3)nn2)c1] | ¹H NMR (400 MHz, DMSO-d₆) δ 16.20 (s, 1H), 15.97 (s, 1H), 8.77 (d, J = 2.8 Hz, 1H), 8.56 (s, 1H), 8.45 (d, J = 2.8 Hz, 1H), 8.44-8.45 (m, 5H), 8.19-8.18 (m, 2H), 7.98 (d, J = 8.8 Hz, 1H), 7.65 (s, 1H), 7.25 (s. 2H), 6.63-6.66 (m, 2H), 4.10-4.11 (m, 4H), 1.59-1.61 (m, 4H). MS-ESI: m/z 735.2 observed [M + H]⁺ |
| 50 | 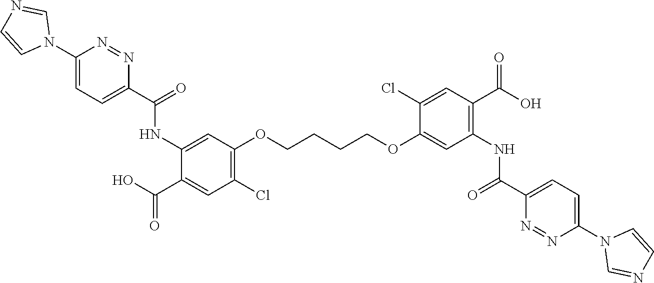 [SMILES: O=C(Nc1cc(OCCCCOc2cc(NC(=O)c3ccc(-n4ccnc4)nn3)c(C(=O)O)cc2Cl)c(Cl)cc1C(=O)O)c1ccc(-n2ccnc2)nn1] | ¹H NMR (400 MHz, DMSO-d₆) δ 15.90 (s, 2H), 8.77 (s, 2H), 8.67 (s, 2H), 8.45 (d, J = 8.8 Hz, 2H), 8.39 (d, J = 9,2 Hz, 2H), 8.18 (s, 2H), 7.98 (s, 2H), 7.25 (s, 2H), 4.23 (s, 4H), 2.03 (s, 4H). MS-ESI: m/z 773.1 observed [M + H]⁺ |
| 51 | 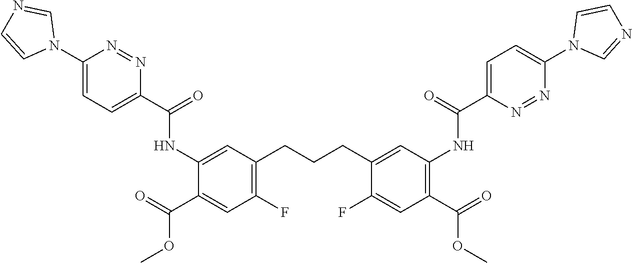 [SMILES: COC(=O)c1cc(F)c(CCCc2cc(NC(=O)c3ccc(-n4ccnc4)nn3)c(C(=O)OC)cc2F)cc1NC(=O)c1ccc(-n2ccnc2)nn1] | ¹H NMR (400 MHz, DMSO-d₆) δ 9.52 (s, 2H), 8.69 (d, J = 9.2 Hz, 2H), 8.50 (d, J = 5.6 Hz, 2H), 8.28 (d, J = 8.0 Hz, 2H), 8.18 (s, 2H), 7.71 (d, J = 10.0 Hz, 2H), 7.61 (s, 2H), 3.92 (s, 6H), 2.76 (t. J = 7.2 Hz, 4H), 2.02-2.00 (m, 2H). MS-ESI: m/z 723.57 observed [M + H]⁺ |
| 52 | 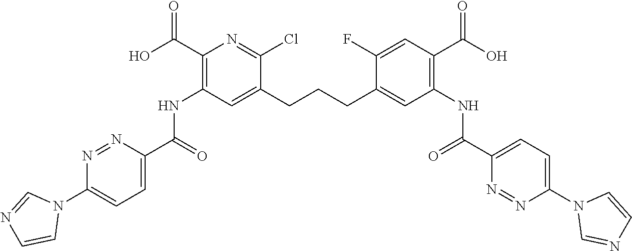 [SMILES: O=C(Nc1cc(CCCc2cc(NC(=O)c3ccc(-n4ccnc4)nn3)c(C(=O)O)nc2Cl)c(F)cc1C(=O)O)c1ccc(-n2ccnc2)nn1] | ¹H NMR (400 MHz, DMSO-d₆) δ 9.14 (s, 1H), 8.75 (dd, J = 13.4, 6.3 Hz, 3H), 8.52- 8.29 (m, 3H), 8.18 (d, J = 4.1 Hz, 2H), 7.69 (d, J = 10.9 Hz, 1H), 7.25 (s, 2H), 2.79 (dt, J = 17.4, 7.6 Hz, 4H), 2.05- 1.87 (m, 2H). MS-ESI: m/z 712.84 observed [M + H]⁺ |
| 53 | 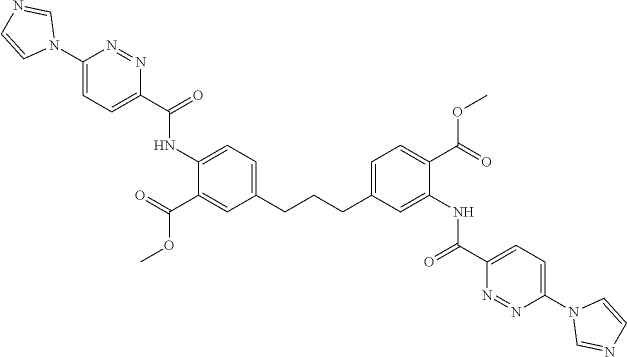 [SMILES: COC(=O)c1ccc(CCCc2ccc(NC(=O)c3ccc(-n4ccnc4)nn3)c(C(=O)OC)c2)cc1NC(=O)c1ccc(-n2ccnc2)nn1] | MS-ESI: m/z 687.5 observed [M + H]⁺ |
| 54 | 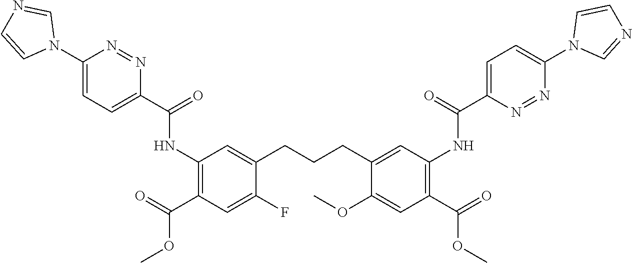 [SMILES: COC(=O)c1cc(F)c(CCCc2cc(NC(=O)c3ccc(-n4ccnc4)nn3)c(C(=O)OC)cc2OC)cc1NC(=O)c1ccc(-n2ccnc2)nn1] | MS-ESI: m/z 735.6 observed [M + H]⁺ |
| 55 | 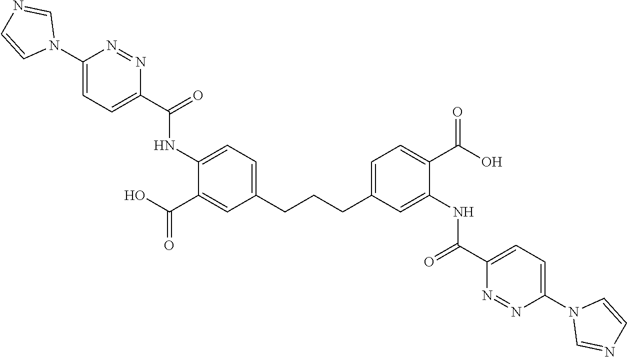 [SMILES: O=C(Nc1cc(CCCc2ccc(NC(=O)c3ccc(-n4ccnc4)nn3)c(C(=O)O)c2)ccc1C(=O)O)c1ccc(-n2ccnc2)nn1] | ¹H NMR (400 MHz, DMSO-d₆) δ 16.04 (s, 1H), 15.93 (s, 1H), 8.79 (s, 2H), 6.66- 6.64 (m, 2H), 8.49 (d, J = 6.0 Hz, 2H), 8.46 (d, J = 6.0 Hz, 2H), 8.42-8.39 (m, 2H), 8.21 (s, 2H), 7.96-7.92 (m, 2H), 7.26 (s, 2H), 7.21 (d, J = 8.4 Hz, 1H), 6.90 (d, J = 7.2 Hz, 1H), 2.68- 2.64 (m, 4H), 1.94- 1.92 (m, 2H). MS-ESI: m/z 659.2 observed [M + H]⁺ |
| 56 | 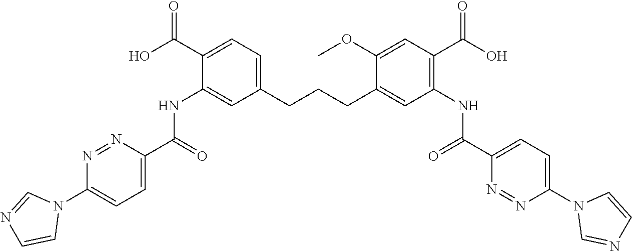 [SMILES: COc1cc(C(=O)O)c(NC(=O)c2ccc(-n3ccnc3)nn2)cc1CCCc1ccc(C(=O)O)c(NC(=O)c2ccc(-n3ccnc3)nn2)c1] | ¹H NMR (400 MHz, DMSO-d₆) δ 8.79 8.74 (m, 2H), 8.63 (s, 2H), 8.43 (dd, J = 9.1, 1.2 Hz, 2H), 8.37 (dd, J = 9.2, 3.9 Hz, 2H), 8.18 (q, J = 1.6 Hz, 2H), 7.95 (d, J = 7.8 Hz, 1H), 7.63 (s, 1H), 7.28-7.23 (m, 2H), 6.94-6.88 (m, 1H), 3.79 (s, 3H), 2.69-2.65 (m, 4H), 2.01-1.83 (m, 2H). MS-ESI: m/z 689.21 observed [M + H]⁺ |
| 57 | 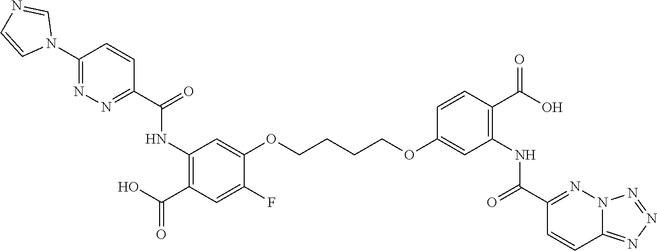 [SMILES: O=C(Nc1cc(OCCCCOc2ccc(C(=O)O)c(NC(=O)c3ccc4nnnn4n3)c2)c(F)cc1C(=O)O)c1ccc(-n2ccnc2)nn1] | ¹H NMR (500 MHz, DMSO-d₆) δ 10.23 (s, 1H), 8.97 (d, J = 9.4 Hz, 1H), 8.82- 8.57 (m, 4H), 8.40- 8.29 (m, 2H), 8.04 (d, J = 10.0 Hz, 2H), 7.77 (d, J = 12.0 Hz, 1H), 6.87 (dd, J = 8.8, 2.5 Hz, 1H), 4.38- 4.28 (m, 2H), 4.23 (t, J = 5.9 Hz, 2H), 2.11-1.94 (m, 4H). MS-ESI: m/z 698.2 observed [M + H]⁺ |
| 58 | 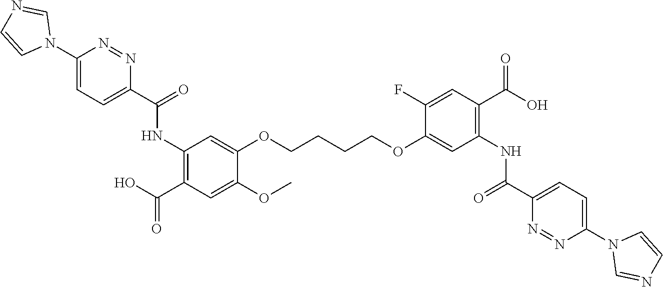 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1F] | ¹H NMR (400 MHz, DMSO-d₆) δ 13.36 (s, 1H), 13.29 (s, 1H), 9.95-9.92 (m, 2H), 8.77 (d. J = 8 Hz, 1H), 8.69-8.59 (m, 7H), 7.88 (s, 1H), 7.81 (d, J = 12.0 Hz, 1H), 7.53 (s, 1H), 4.33 (s, 2H), 4.23 (s, 2H), 3.80 (s, 3H), 2.05 (s, 4H). MS-ESI: m/z 753.3 observed [M + H]⁺ |
| 59 | 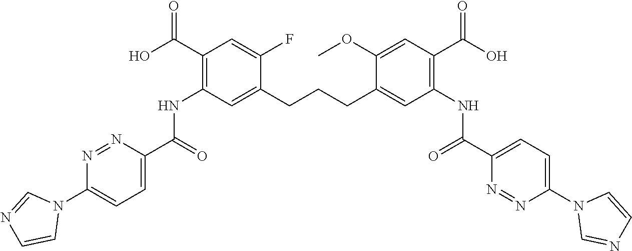 [SMILES: COc1cc(C(=O)O)c(NC(=O)c2ccc(-n3ccnc3)nn2)cc1CCCc1cc(NC(=O)c2ccc(-n3ccnc3)nn2)c(C(=O)O)cc1F] | ¹H NMR (400 MHz, DMSO-d₆) δ 8.76 (dt, J = 2.4, 1.1 Hz, 2H), 8.72 (d, J = 7.3 Hz, 1H), 8.62 (s, 1H), 8.43 (dd, J = 9,1, 5.7 Hz, 2H), 8.37 (dd, J = 9.2, 4,6 Hz, 2H), 8.18 (q, J = 1.6 Hz, 2H), 7.67 (d, J = 11.0 Hz, 1H), 7.64 (s, 1H), 7.28-7.21 (m, 2H), 3.79 (s, 3H), 2.75-2.67 (m, 4H), 1.95-1.84 (m, 2H). MS-ESI: m/z 707.37 observed [M + H]⁺ |
| 60 | 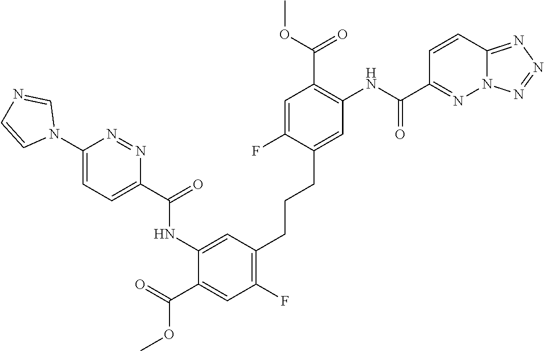 | MS-ESI: m/z 707.65 observed [M + H]⁺ |
| 61 | 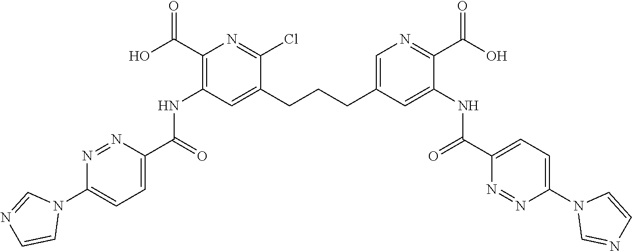 [SMILES: O=C(Nc1cc(CCCc2cc(NC(=O)c3ccc(-n4ccnc4)nn3)c(C(=O)O)nc2Cl)cnc1C(=O)O)c1ccc(-n2ccnc2)nn1] | MS-ESI: m/z 698.8 observed [M + H]⁺ |
| 62 | 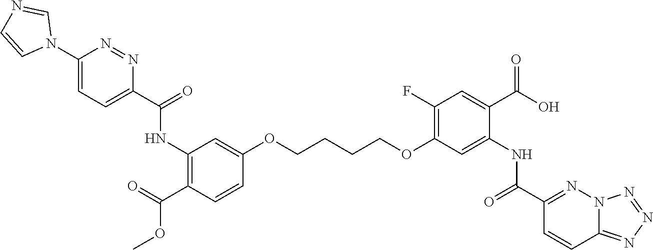 [SMILES: COC(=O)c1ccc(OCCCCOc2cc(NC(=O)c3ccc4nnnn4n3)c(C(=O)O)cc2F)cc1NC(=O)c1ccc(-n2ccnc2)nn1] | ¹H NMR (400 MHz, DMSO-d₆) δ 15.55 (s, 1H), 15.44 (s, 1H), 9.11 (s, 2H), 8.79 (s, 2H), 8.48-8.38 (m, 4H), 8.20 (s, 2H), 7.26 (s, 2H), 2.82-2.80 (m, 2H), 2.0-1.98 (m, 2H), 1.55 (s, 2H). MS-ESI: m/z 712.2 observed [M + H]⁺ |
| 63 | 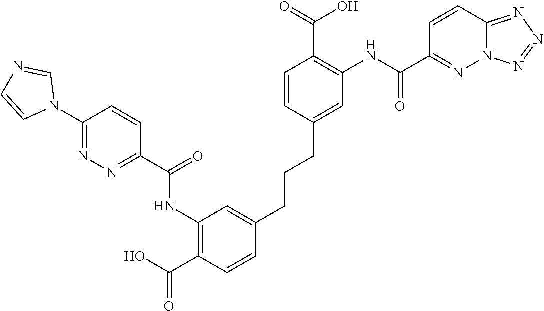 [SMILES: O=C(Nc1cc(CCCc2ccc(C(=O)O)c(NC(=O)c3ccc4nnnn4n3)c2)ccc1C(=O)O)c1ccc(-n2ccnc2)nn1] | MS-ESI: m/z 634.42 observed [M + H]⁺ |
| 64 | 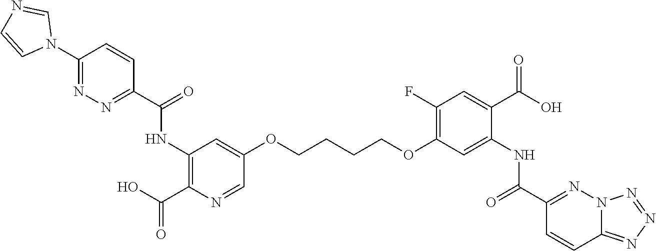 [SMILES: O=C(Nc1cc(OCCCCOc2cnc(C(=O)O)c(NC(=O)c3ccc(-n4ccnc4)nn3)c2)c(F)cc1C(=O)O)c1ccc2nnnn2n1] | ¹H NMR (500 MHz, DMSO-d₆) δ 10.24 (s, 1H), 8.98 (d, J = 9.3 Hz, 1H), 8.80 (s, 1H), 8.76-8.58 (m, 4H), 8.37 (d, J = 9.1 Hz, 1H), 8.22 (s, 1H), 8.02 (s, 1H), 7.78 (d, J = 11.7 Hz, 1H), 4.39-4.22 (m, 3H), 2.10-1.94 (m, 4H). MS-ESI: m/z 699.1 observed [M + H]⁺ |
| 65 | 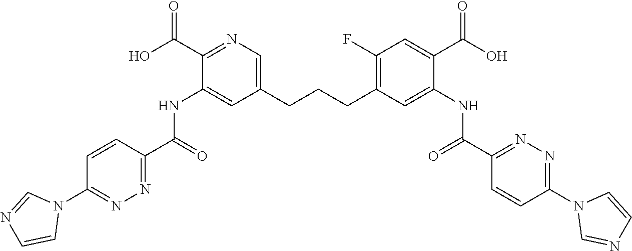 [SMILES: O=C(Nc1cc(CCCc2cnc(C(=O)O)c(NC(=O)c3ccc(-n4ccnc4)nn3)c2)c(F)cc1C(=O)O)c1ccc(-n2ccnc2)nn1] | ¹H NMR (400 MHz, DMSO-d₆) δ 15.85 (s, 1H), 15.37 (s, 1H), 9.13 (s, 1H), 8.81- 8.73 (m, 3H), 8.50- 8.38 (m, 4H), 8.25- 8.21 (m, 3H), 7.70 (d, J = 10.8 Hz, 1H), 7.27 (d, J = 3.2 Hz, 2H), 2.81-2.52 (m, 4H), 2.01-1.99 (m, 2H). MS-ESI: m/z 678.61 observed [M + H]⁺ |
| 66 | 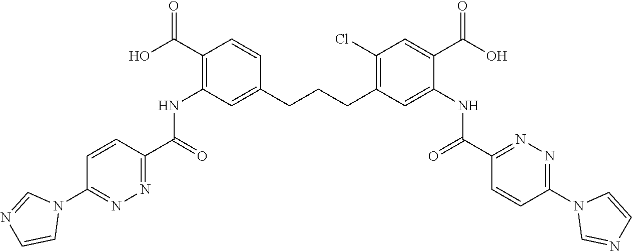 [SMILES: O=C(Nc1cc(CCCc2cc(NC(=O)c3ccc(-n4ccnc4)nn3)c(C(=O)O)cc2Cl)ccc1C(=O)O)c1ccc(-n2ccnc2)nn1] | ¹H NMR (400 MHz, DMSO-d₆) δ 15.98 (s, 1H), 15.83 (s, 1H), 8.80 (s, 3H), 8.79 (s, 1H), 8.48-8.39 (m, 4H), 8.20 (s, 2H), 8.02-7.99 (m, 2H), 7.27 (s, 2H), 6.96 (d, J = 7.6 Hz, 1H), 2.81- 2.69 (m, 4H), 2.0- 1.98 (m, 2H). MS-ESI: m/z 693.8 observed [M + H]⁺ |
| 67 | 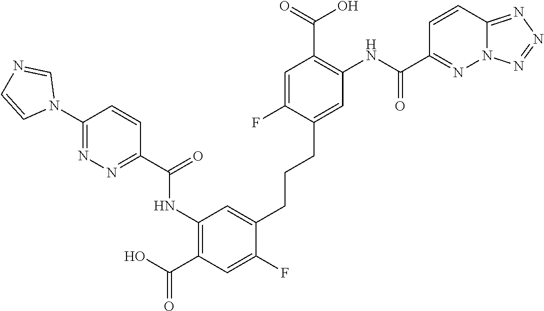 [SMILES: O=C(Nc1cc(CCCc2cc(NC(=O)c3ccc4nnnn4n3)c(C(=O)O)cc2F)c(F)cc1C(=O)O)c1ccc(-n2ccnc2)nn1] | ¹H NMR (400 MHz, DMSO-d₆) δ 8.91 (d, J = 9.5 Hz, 1H), 8.77 (s, 1H), 8.70 (dd, J = 19.8. 7.2 Hz, 2H), 8.48-8.28 (m, 3H), 8.19 (d, J = 1.5 Hz, 1H), 7.68 (dd, J = 10.9, 6.4 Hz, 2H), 7.25 (s, 1H), 2.77- 2.69 (m, 4H), 1.99- 1.87 (m, 2H). MS-ESI: m/z 670.57 observed [M + H]⁺ |
| 68 | 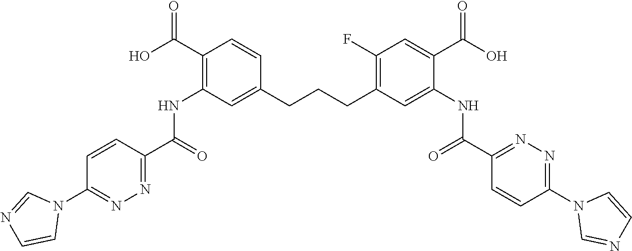 [SMILES: O=C(Nc1cc(CCCc2cc(NC(=O)c3ccc(-n4ccnc4)nn3)c(C(=O)O)cc2F)ccc1C(=O)O)c1ccc(-n2ccnc2)nn1] | ¹H NMR (500 MHz, DMSO-d₆) δ 8.77 (s, 2H), 8.72 (d, J = 7.2 Hz, 1H), 8.63 (s, 1H), 8.44 (dd, J = 9.1, 5.4 Hz, 2H), 8.37 (d, J = 9.1 Hz, 2H), 8.18 (s, 2H), 7.95 (d, J = 7.9 Hz, 1H), 7.67 (d, J = 10.8 Hz, 1H), 7.25 (s, 2H), 6.92 (d, J = 8.0 Hz, 1H), 2.72-2.66 (m, 5H), 2.00-1.87 (m, 2H). MS-ESI: m/z 677.2 observed [M + H]⁺ |
| 69 | 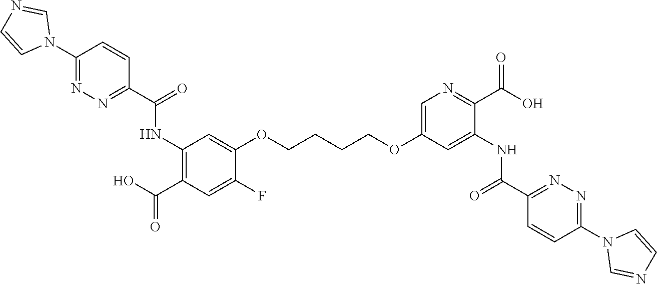 [SMILES: O=C(Nc1cc(OCCCCOc2cnc(C(=O)O)c(NC(=O)c3ccc(-n4ccnc4)nn3)c2)c(F)cc1C(=O)O)c1ccc(-n2ccnc2)nn1] | MS-ESI: m/z 724.1 observed [M + H]⁺ |
| 70 | 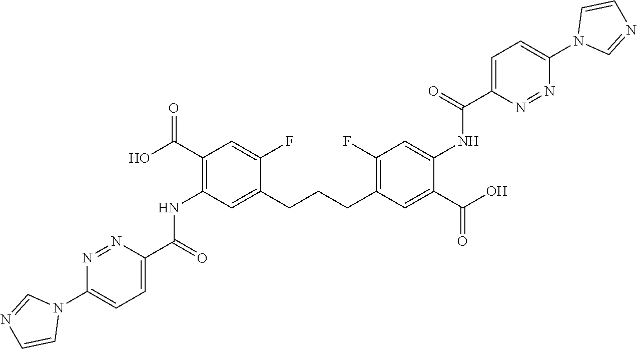 [SMILES: O=C(Nc1cc(CCCc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 8.75 (dd, J = 25.9, 6.1 Hz, 3H), 8.58-8.32 (m, 5H), 8.20 (d, J = 4.5 Hz, 2H), 8.00 (d, J = 9.3 Hz, 1H), 7.66 (d, J = 11.1 Hz, 1H), 7.25 (s, 2H), 1.99- 1.78 (m, 4H), 1.32- 1.17 (m, 2H). MS-ESI: m/z 695.18 observed [M + H]⁺ |
| 71 | 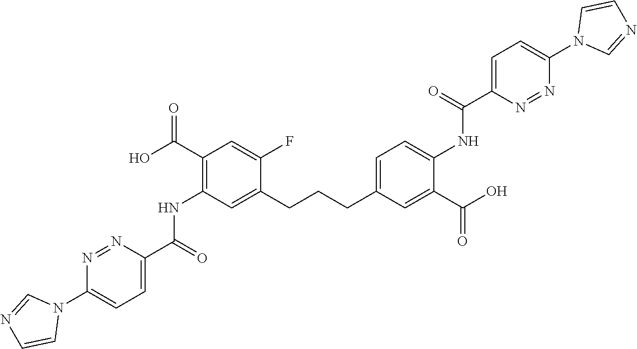 [SMILES: O=C(Nc1ccc(CCCc2cc(NC(=O)c3ccc(-n4ccnc4)nn3)c(C(=O)O)cc2F)cc1C(=O)O)c1ccc(-n2ccnc2)nn1] | ¹H NMR (400 MHz, DMSO-d₆) δ 14.78 (s, 1H), 13.67 (s, 1H), 8.79 (dd, J = 16.8, 6.4 Hz, 4H), 8.55- 8.42 (m, 5H), 8.22 (d, J = 6.4 Hz, 2H), 7.96 (s, 1H), 7.72 (d, J = 10.8 Hz, 1H), 7.55- 7.52 (m, 1H), 7.27 (s, 2H), 2.74-2.69 (m, 4H), 1.99-1.96 (m, 2H). MS-ESI: m/z 677.6 observed [M + H]⁺ |
| 72 | 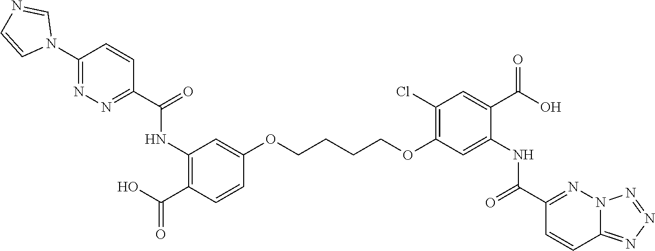 [SMILES: O=C(Nc1cc(OCCCCOc2cc(NC(=O)c3ccc4nnnn4n3)c(C(=O)O)cc2Cl)ccc1C(=O)O)c1ccc(-n2ccnc2)nn1] | ¹H NMR (400 MHz, DMSO-d₆) δ 10.21 (s, 1H), 9.00 (d, J = 9.4 Hz, 1H), 8.73- 8.57 (m, 4H), 8.45 (d, J = 2.5 Hz, 1H), 8.37 (d, J = 9.4 Hz, 1H), 8.06- 7.98 (m, 3H), 6.86 (dd, J = 8.9, 2.5 Hz, 1H), 4.28 (dd, J = 28.1, 6.1 Hz, 4H), 2.05 (d, J = 6.5 Hz, 4H). MS-ESI: m/z 714.16 observed [M + H]⁺ |
| 73 | 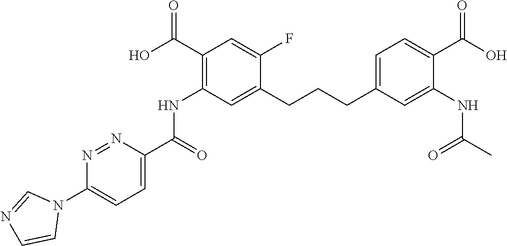 [SMILES: CC(=O)Nc1cc(CCCc2cc(NC(=O)c3ccc(-n4ccnc4)nn3)c(C(=O)O)cc2F)ccc1C(=O)O] | MS-ESI: m/z 547.19 observed [M + H]⁺ |
| 74 | 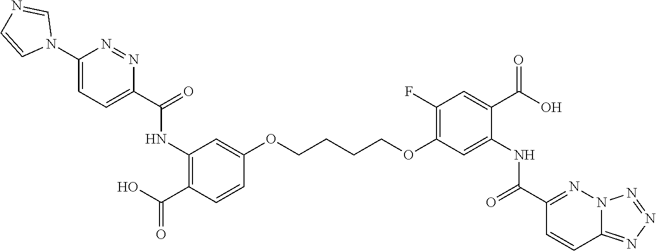 [SMILES: O=C(Nc1cc(OCCCCOc2cc(NC(=O)c3ccc4nnnn4n3)c(C(=O)O)cc2F)ccc1C(=O)O)c1ccc(-n2ccnc2)nn1] | MS-ESI: m/z 698.2 observed [M + H]⁺ |
| 75 | 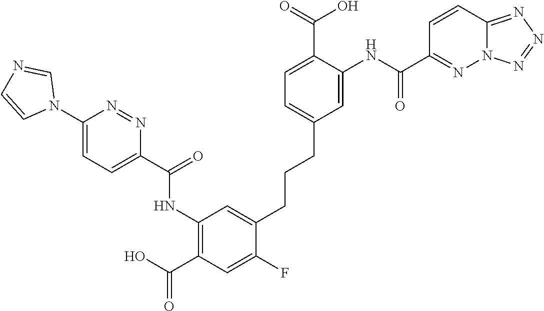 [SMILES: O=C(Nc1cc(CCCc2ccc(C(=O)O)c(NC(=O)c3ccc4nnnn4n3)c2)c(F)cc1C(=O)O)c1ccc(-n2ccnc2)nn1] | MS-ESI: m/z 652.21 observed [M + H]⁺ |
| 76 | 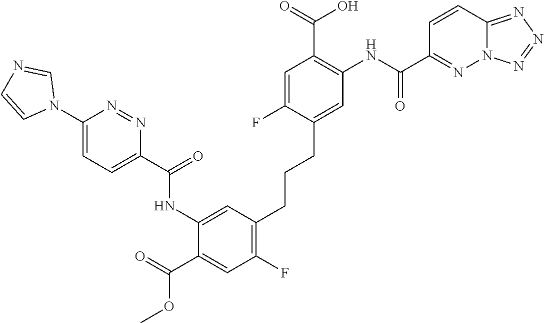 [SMILES: COC(=O)c1cc(F)c(CCCc2cc(NC(=O)c3ccc4nnnn4n3)c(C(=O)O)cc2F)cc1NC(=O)c1ccc(-n2ccnc2)nn1] | MS-ESI: m/z 684.19 observed [M + H]⁺ |
| 77 | 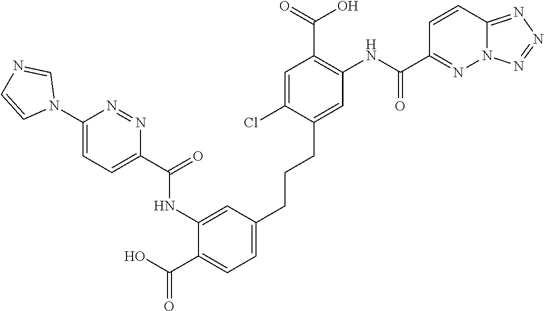 [SMILES: O=C(Nc1cc(CCCc2cc(NC(=O)c3ccc4nnnn4n3)c(C(=O)O)cc2Cl)ccc1C(=O)O)c1ccc(-n2ccnc2)nn1] | ¹H NMR (400 MHz, DMSO-d₆) δ 8.91 (d, J = 9.4 Hz, 1H), 8.77 (t, J = 1.1 Hz, 1H), 8.72 (s, 1H), 8.64 (d, J = 1.7 Hz, 1H), 8.43 (d, J = 9.1 Hz, 1H), 8.37 (d, J = 9.2 Hz, 1H), 8.34 (d, J = 9.5 Hz, 1H), 8.19 (t, J = 1.5 Hz, 1H), 8.02-7.93 (m, 2H), 7.25 (t, J = 1.2 Hz, 1H), 6.94 (dd, J = 8.0, 1.8 Hz, 1H), 2.75 (dt, J = 23.8, 7.7 Hz, 5H), 1.95 (t, J = 8.0 Hz, 2H). MS-ESI: m/z 668.14 observed [M + H]⁺ |
| 78 | 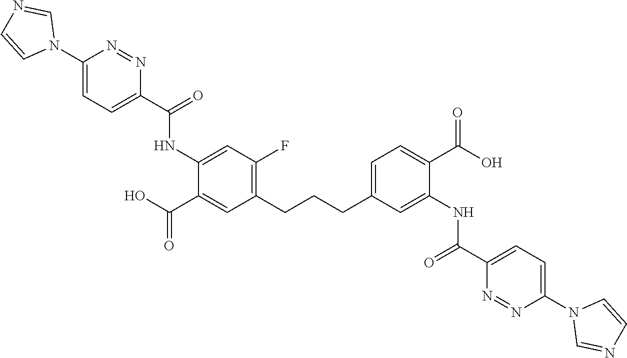 [SMILES: O=C(Nc1cc(CCCc2cc(C(=O)O)c(NC(=O)c3ccc(-n4ccnc4)nn3)cc2F)ccc1C(=O)O)c1ccc(-n2ccnc2)nn1] | ¹H NMR (500 MHz, DMSO-d₆) δ 8.77 (s, 2H), 8.72 (d, J = 7.3 Hz, 1H), 8.63 (s, 1H), 8.43 (dd, J = 9.2, 5.3 Hz, 2H), 8.37 (d, J = 9.2 Hz, 2H), 8.18 (s, 2H), 7.95 (d, J = 7.9 Hz, 1H), 7.67 (d, J = 10.9 Hz, 1H), 7.25 (s, 2H), 6.92 (d, J = 8.0 Hz, 1H), 2.69 (s, 4H), 1.95 (d, J = 11.2 Hz, 2H). MS-ESI: m/z 676.9 observed [M + H]⁺ |
| 79 | 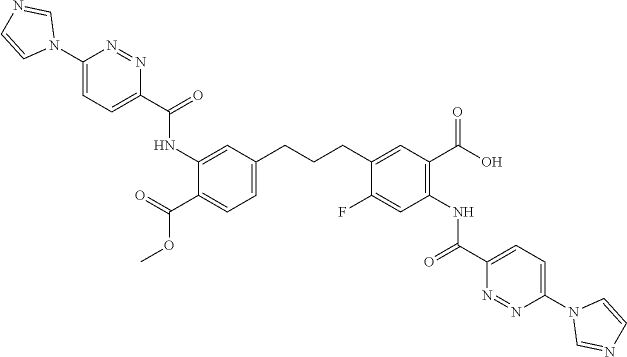 [SMILES: COC(=O)c1ccc(CCCc2cc(C(=O)O)c(NC(=O)c3ccc(-n4ccnc4)nn3)cc2F)cc1NC(=O)c1ccc(-n2ccnc2)nn1] | MS-ESI: m/z 691.0 observed [M + H]⁺ |
| 80 | 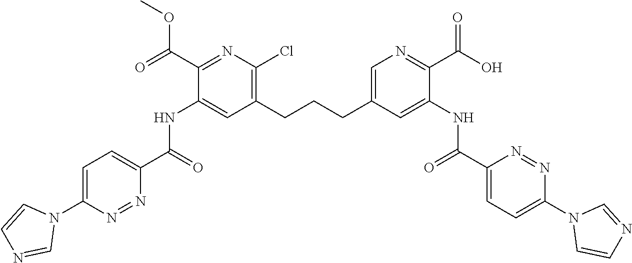 [SMILES: COC(=O)c1nc(Cl)c(CCCc2cnc(C(=O)O)c(NC(=O)c3ccc(-n4ccnc4)nn3)c2)cc1NC(=O)c1ccc(-n2ccnc2)nn1] | MS-ESI: m/z 709.19 observed [M + H]⁺ |
| 81 | 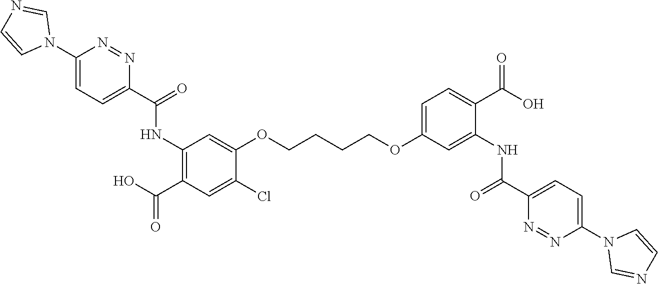 [SMILES: O=C(Nc1cc(OCCCCOc2cc(NC(=O)c3ccc(-n4ccnc4)nn3)c(C(=O)O)cc2Cl)ccc1C(=O)O)c1ccc(-n2ccnc2)nn1] | ¹H NMR (400 MHz, DMSO-d₆) δ 13.42 (s, 2H), 9.70-9.50 (m, 2H), 8.76-8.63 (m, 7H), 8.04-8.02 (m, 2H), 7.94-7.92 (m, 2H), 6.87 (dd, J = 8.8, 2.4 Hz, 1H), 4.32-4.25 (m, 4H), 2.33-2.04 (m, 4H). MS-ESI: m/z 739.4 observed [M + H]⁺ |
| 82 | 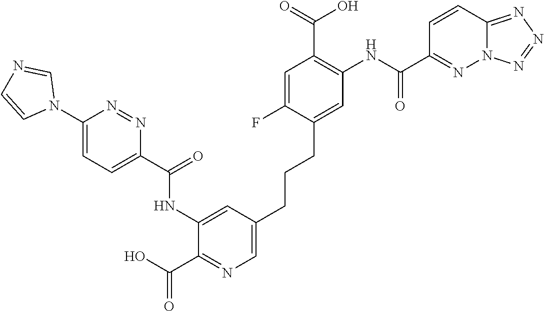 [SMILES: O=C(Nc1cc(CCCc2cnc(C(=O)O)c(NC(=O)c3ccc(-n4ccnc4)nn3)c2)c(F)cc1C(=O)O)c1ccc2nnnn2n1] | ¹H NMR (400 MHz, DMSO-d₆) δ 9.03- 8.92 (m, 2H), 8.79- 8.75 (m, 2H), 8.47- 8.34 (m, 3H), 8.20 (s, 1H), 7.71 (d, J = 10.8 Hz, 1H), 7.27 (s, 2H), 2.82-2.69 (m, 4H), 2.11-1.93 (m, 2H). MS-ESI: m/z 653.3 observed [M + H]⁺ |
| 83 | 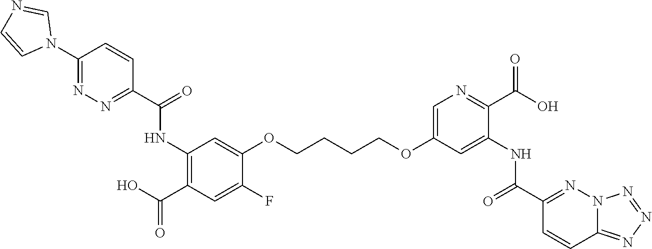 [SMILES: O=C(Nc1cc(OCCCCOc2cnc(C(=O)O)c(NC(=O)c3ccc4nnnn4n3)c2)c(F)cc1C(=O)O)c1ccc(-n2ccnc2)nn1] | MS-ESI: m/z 699.43 observed [M + H]⁺ |
| 84 | 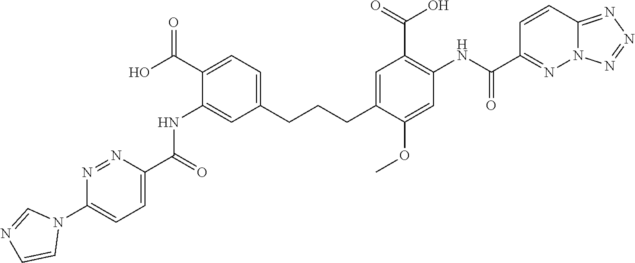 [SMILES: COc1cc(NC(=O)c2ccc3nnnn3n2)c(C(=O)O)cc1CCCc1ccc(C(=O)O)c(NC(=O)c2ccc(-n3ccnc3)nn2)c1] | ¹H NMR (400 MHz, DMSO-d₆) δ 9.00 (d, J = 9.6 Hz, 1H), 8.79 (s, 1H), 8.71 (s, 1H), 8.51-8.37 (m, 4H), 8.19 (s, 1H), 8.01-7.92 (m, 1H), 7.89 (s, 1H), 7.27 (s, 1H), 7.10-7.05 (m, 1H), 3.90 (s, 3H), 2.78-2.62 (m, 4H), 1.98-1.90 (m, 2H). MS-ESI: m/z 664.1 observed [M + H]⁺ |
| 85 | 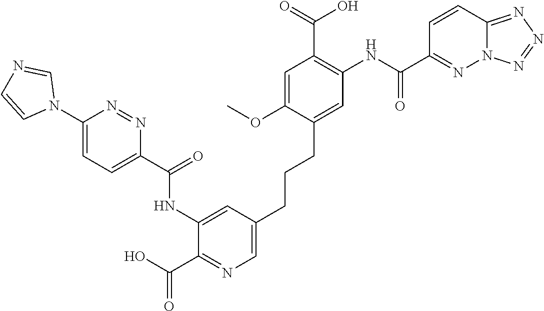 [SMILES: COc1cc(C(=O)O)c(NC(=O)c2ccc3nnnn3n2)cc1CCCc1cnc(C(=O)O)c(NC(=O)c2ccc(-n3ccnc3)nn2)c1] | ¹H NMR (400 MHz, DMSO-d₆) δ 9.07 (s, 1H), 8.94-8.92 (m, 1H), 8.79 (s, 1H), 8.60 (s, 1H), 8.45- 8.34 (m, 3H), 8.19 (s, 1H), 7.59 (s. 1H), 7.25 (s, 2H), 3.83 (s, 3H), 2.81-2.70 (s, 4H), 2.01-1.98 (m, 2H). MS-ESI: m/z 665.2 observed [M + H]⁺ |
| 86 | 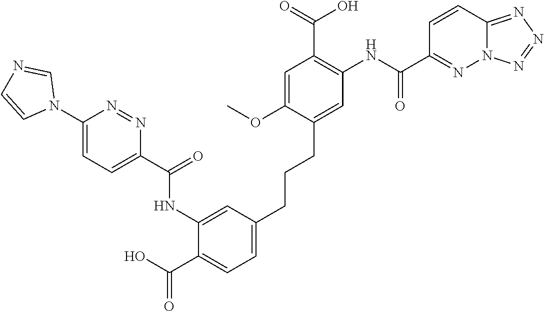 [SMILES: COc1cc(C(=O)O)c(NC(=O)c2ccc3nnnn3n2)cc1CCCc1ccc(C(=O)O)c(NC(=O)c2ccc(-n3ccnc3)nn2)c1] | ¹H NMR (400 MHz, DMSO-d₆) δ 16.19 (d, J = 18 Hz, 1H), 15.75 (d, J = 22.4 Hz, 1H), 8.91 (d, J = 9.2 Hz, 1H), 8.78 (s, 1H), 8.65 (s, 1H), 8.59 (s, 1H), 8.44- 8.33 (m, 3H), 8.19 (s, 1H), 7.97-7.95 (m, 1H), 7.66 (s, 1H), 7.26 (s, 1H), 6.95 (d, J = 7.2 Hz, 1H), 3.81 (s, 3H), 2.71- 2.65 (m, 4H), 1.94- 1.91 (m, 2H). MS-ESI: m/z 664.2 observed [M + H]⁺ |
| 87 | 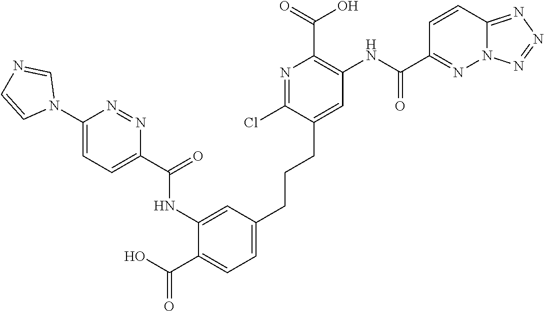 [SMILES: O=C(Nc1cc(CCCc2cc(NC(=O)c3ccc4nnnn4n3)c(C(=O)O)nc2Cl)ccc1C(=O)O)c1ccc(-n2ccnc2)nn1] | ¹H NMR (400 MHz, DMSO-d₆) δ 16.10 (d, J = 19.6 Hz, 1H), 8.79 (s, 1H), 8.70- 8.64 (m, 2H), 8.46- 8.38 (m, 2H), 8.20- 8.11 (m, 2H), 7.95 (d, J = 8 Hz, 1H), 7.26 (s, 1H), 7.09-6.91 (m, 2H), 1.92-1.90 (m, 3H), 1.76 (s, 1H), 1.24-1.17 (m, 3H). MS-ESI: m/z 669.7 observed [M + H]⁺ |
| 88 | 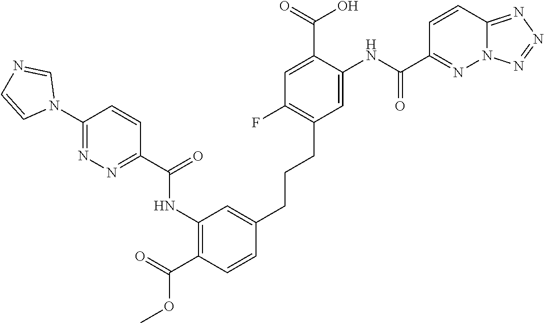 [SMILES: COC(=O)c1ccc(CCCc2cc(NC(=O)c3ccc4nnnn4n3)c(C(=O)O)cc2F)cc1NC(=O)c1ccc(-n2ccnc2)nn1] | MS-ESI: m/z 666.2 observed [M + H]⁺ |
| 89 | 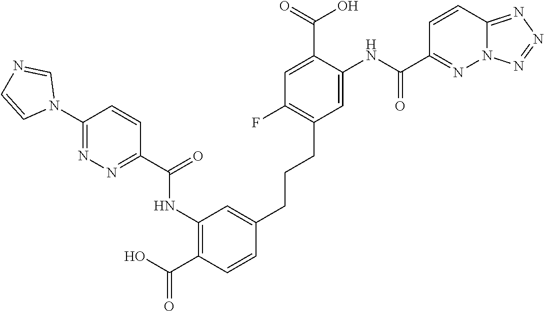 [SMILES: O=C(Nc1cc(CCCc2cc(NC(=O)c3ccc4nnnn4n3)c(C(=O)O)cc2F)ccc1C(=O)O)c1ccc(-n2ccnc2)nn1] | MS-ESI: m/z 652.17 observed [M + H]⁺ |
| 90 | 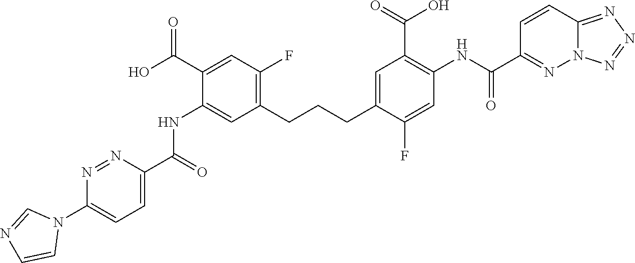 [SMILES: O=C(Nc1cc(CCCc2cc(C(=O)O)c(NC(=O)c3ccc4nnnn4n3)cc2F)c(F)cc1C(=O)O)c1ccc(-n2ccnc2)nn1] | MS-ESI: m/z 670.19 observed [M + H]⁺ |
| 91 | 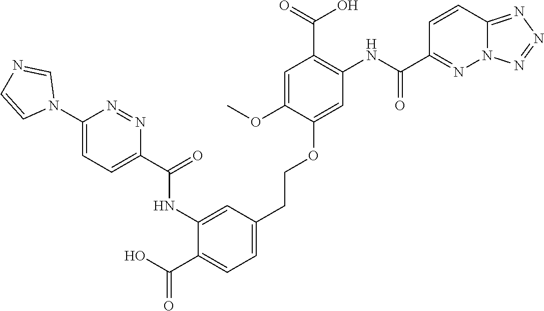 [SMILES: COc1cc(C(=O)O)c(NC(=O)c2ccc3nnnn3n2)cc1OCCc1ccc(C(=O)O)c(NC(=O)c2ccc(-n3ccnc3)nn2)c1] | MS-ESI: m/z 666.2 observed [M + H]⁺ |
| 92 | 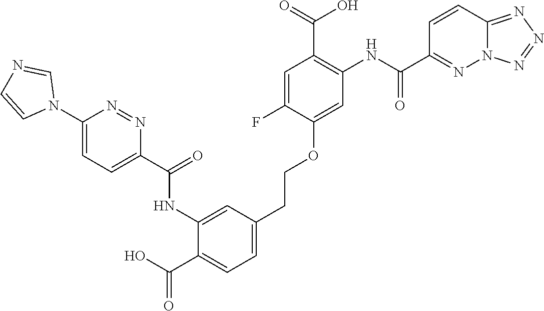 [SMILES: O=C(Nc1cc(CCOc2cc(NC(=O)c3ccc4nnnn4n3)c(C(=O)O)cc2F)ccc1C(=O)O)c1ccc(-n2ccnc2)nn1] | ¹HNMR (400 MHz, DMSO-d₆) δ 15.93 (s, 1H), 14.21 (s, 1H), 8.94 (d, J = 9.6 Hz, 1H), 8.83-8.80 (m, 2H), 8,80-8.78 (m, 1H), 8.49-8.43 (m, 2H), 8.34 (d, J = 9.2 Hz, 1H), 8.19 (s, 1H), 8.05-8.03 (m, 1H), 7.76 (d, J = 12.4 Hz, 1H), 7.26- 7.11 (m, 2H), 4.39- 4.37 (m, 2H), 3.22 (t, J = 6.8 Hz, 1H). MS-ESI: m/z 653.9 observed [M + H]⁺ |
| 93 | 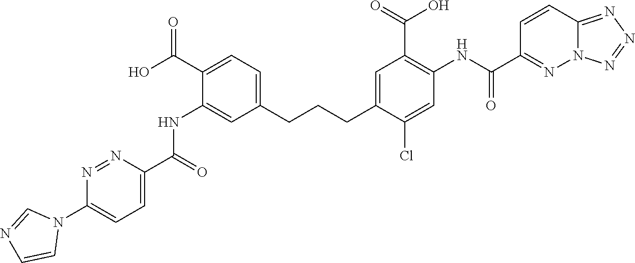 [SMILES: O=C(Nc1cc(CCCc2cc(C(=O)O)c(NC(=O)c3ccc4nnnn4n3)cc2Cl)ccc1C(=O)O)c1ccc(-n2ccnc2)nn1] | ¹H NMR (400 MHz, DMSO-d₆) δ 8.95 (d, J = 9.6 Hz, 1H), 8.80- 8.74 (m, 3H), 8.52- 8.45 (m, 2H), 8.35 (d, J = 9.6 Hz, 1H), 8.21 (s, 1H), 9.08 (s, 1H), 8.02-8.00 (m, 1H), 7.26 (s, 1H), 7.08 (d, J = 8.8 Hz, 1H), 2.80-2.76 (m, 4H), 1.97-1.94 (m, 2H). MS-ESI: m/z 668.4 observed [M + H]⁺ |
| 94 | 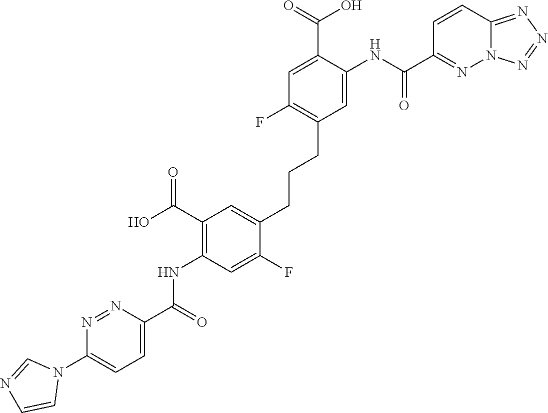 [SMILES: O=C(Nc1cc(F)c(CCCc2cc(NC(=O)c3ccc4nnnn4n3)c(C(=O)O)cc2F)cc1C(=O)O)c1ccc(-n2ccnc2)nn1] | MS-ESI: m/z 670.17 observed [M + H]⁺ |
| 95 | 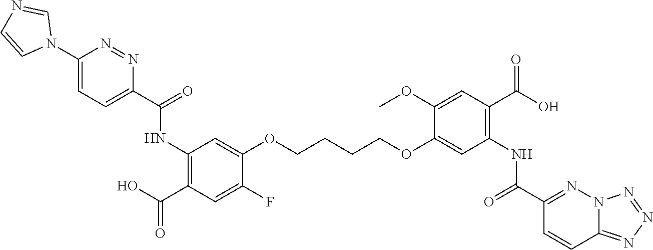 [SMILES: COc1cc(C(=O)O)c(NC(=O)c2ccc3nnnn3n2)cc1OCCCCOc1cc(NC(=O)c2ccc(-n3ccnc3)nn2)c(C(=O)O)cc1F] | ¹H NMR (400 MHz, DMSO-d₆) δ 8.97 (d, J = 9.6 Hz, 1H), 8.79 (s, 1H), 8.74-8.74 (m, 1H), 8.54 (s, 1H), 8.45 (d, J = 16.4 Hz, 1H), 8.42-8.41 (m, 1H), 8.36 (d, J = 9.6 Hz, 1H), 8.19 (s, 1H), 7.78 (d, J = 9.0 Hz, 1H), 7.57 (s, 1H), 7.27 (s, 1H), 4.29-4.27 (m, 2H), 4.21-4.19 (m, 2H), 3.79 (s, 3H), 2.03 (s, 4H). MS-ESI: m/z 728.19 observed [M + H]⁺ |
| 96 | 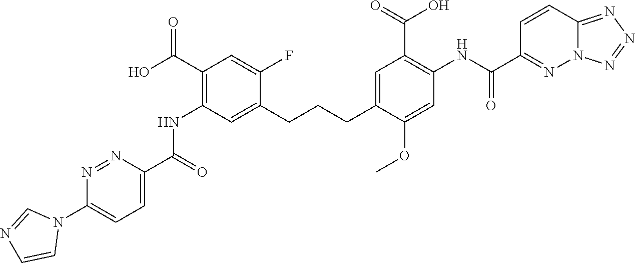 [SMILES: COc1cc(NC(=O)c2ccc3nnnn3n2)c(C(=O)O)cc1CCCc1cc(NC(=O)c2ccc(-n3ccnc3)nn2)c(C(=O)O)cc1F] | MS-ESI: m/z 682.3 observed [M + H]⁺ |
| 97 | 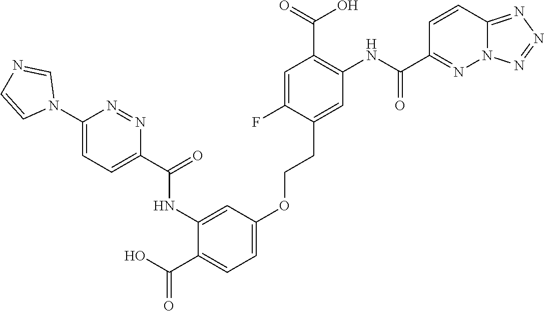 [SMILES: O=C(Nc1cc(OCCc2cc(NC(=O)c3ccc4nnnn4n3)c(C(=O)O)cc2F)ccc1C(=O)O)c1ccc(-n2ccnc2)nn1] | MS-ESI: m/z 654.15 observed [M + H]⁺ |
| 98 | 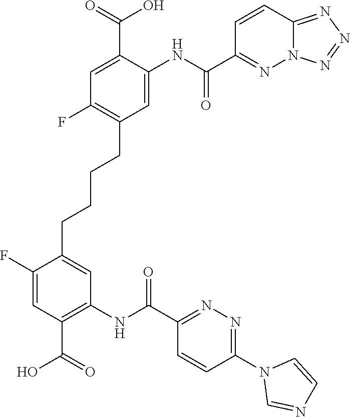 [SMILES: O=C(Nc1cc(CCCCc2cc(NC(=O)c3ccc4nnnn4n3)c(C(=O)O)cc2F)c(F)cc1C(=O)O)c1ccc(-n2ccnc2)nn1] | MS-ESI: m/z 684.18 observed [M + H]⁺ |
| 99 | 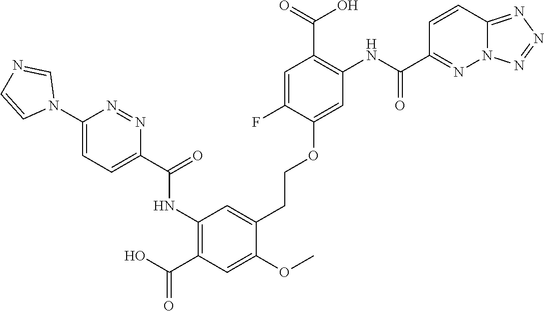 [SMILES: COc1cc(C(=O)O)c(NC(=O)c2ccc(-n3ccnc3)nn2)cc1CCOc1cc(NC(=O)c2ccc3nnnn3n2)c(C(=O)O)cc1F] | MS-ESI: m/z 684.4 observed [M + H]⁺ |
| 100 | 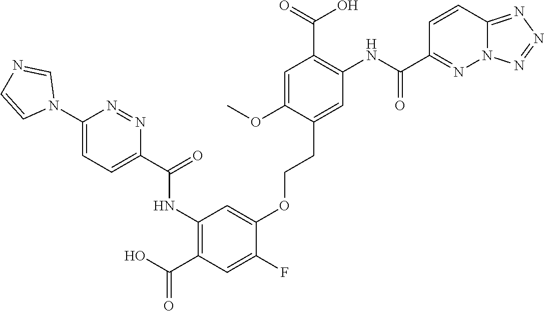 [SMILES: COc1cc(C(=O)O)c(NC(=O)c2ccc3nnnn3n2)cc1CCOc1cc(NC(=O)c2ccc(-n3ccnc3)nn2)c(C(=O)O)cc1F] | ¹H NMR (500 MHz, DMSO-d₆) δ 8.90 (d, J = 9.5 Hz, 1H), 8.77 (s, 1H), 8.73-8.62 (m, 2H), 8.48-8.36 (m, 2H), 8.33 (d, J = 9.4 Hz, 1H), 8.18 (s, 1H), 7.72 (d, J = 13.5 Hz, 2H), 7.25 (s, 1H), 4.24 (t, J = 7.4 Hz, 2H), 3.85 (s, 3H), 3.13 (t, J = 7.4 Hz, 2H). MS-ESI: m/z 684.16 observed [M + H]⁺ |
| 101 | 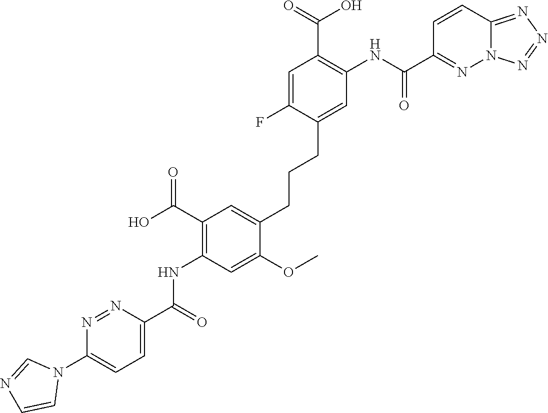 [SMILES: COc1cc(NC(=O)c2ccc(-n3ccnc3)nn2)c(C(=O)O)cc1CCCc1cc(NC(=O)c2ccc3nnnn3n2)c(C(=O)O)cc1F] | MS-ESI: m/z 682.0 observed [M + H]⁺ |
| 102 | 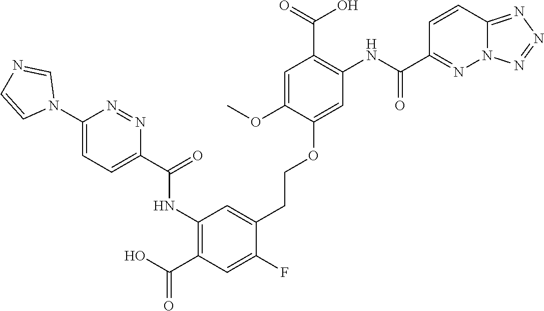 [SMILES: COc1cc(C(=O)O)c(NC(=O)c2ccc3nnnn3n2)cc1OCCc1cc(NC(=O)c2ccc(-n3ccnc3)nn2)c(C(=O)O)cc1F] | MS-ESI: m/z 684.16 observed [M + H]⁺ |
| 103 | 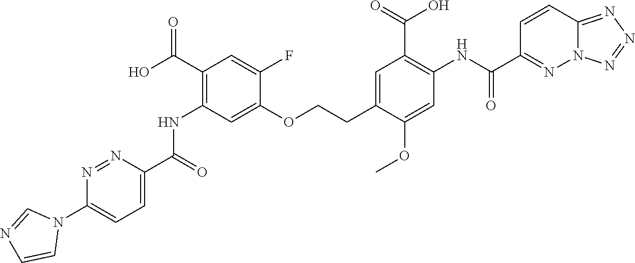 [SMILES: COc1cc(NC(=O)c2ccc3nnnn3n2)c(C(=O)O)cc1CCOc1cc(NC(=O)c2ccc(-n3ccnc3)nn2)c(C(=O)O)cc1F] | ¹H NMR (500 MHz, DMSO-d₆) δ 8.92 (d. J = 9.5 Hz, 1H), 8.83 (s, 1H), 8.76 (s, 1H), 8.60 (s, 1H), 8.45 (d, J = 9.1 Hz, 1H), 8.38 (d, J = 9.1 Hz, 1H), 8.33 (d, J = 9.4 Hz, 1H), 8.17 (s, 1H), 8.05 (s, 1H), 7.62 (s, 1H), 7.25 (s, 1H), 4.25 (d, J = 8.0 Hz, 2H), 3.76 (s, 3H), 3.26 (d, J = 7.0 Hz, 3H). MS-ESI: m/z 684.4 observed [M + H]⁺ |
| 104 | 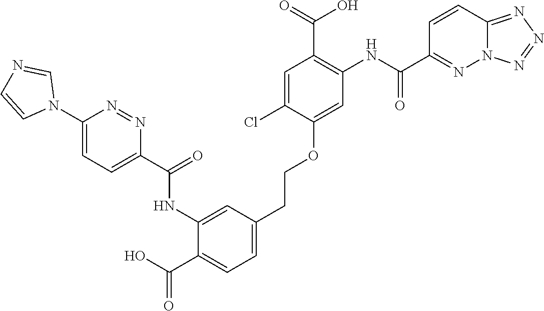 [SMILES: O=C(Nc1cc(CCOc2cc(NC(=O)c3ccc4nnnn4n3)c(C(=O)O)cc2Cl)ccc1C(=O)O)c1ccc(-n2ccnc2)nn1] | MS-ESI: m/z 670.12 observed [M + H]⁺ |
| 105 | 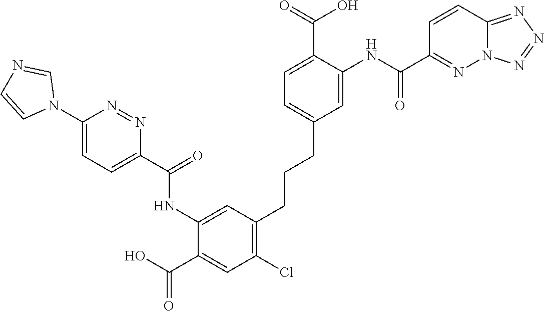 [SMILES: O=C(Nc1cc(CCCc2ccc(C(=O)O)c(NC(=O)c3ccc4nnnn4n3)c2)c(Cl)cc1C(=O)O)c1ccc(-n2ccnc2)nn1] | ¹H NMR (500 MHz, DMSO-d₆) δ 13.21 (s, 1H) 13.14 (s, 1H), 9.17 (s, 1H), 8.99 (d, J = 9.2 Hz, 1H), 8.87 (s, 1H), 8.70 (s, 1H), 8.58 (d, J = 7.2 Hz, 1H), 8.48 (d, J = 8.8 Hz, 1H), 8.35- 8.33 (m, 2H), 8.11- 7.97 (m, 2H), 7.47 (s, 1H), 7.22 (d, J = 8.4 Hz, 1H), 2.88- 2.83 (m, 4H), 2.07- 2.03 (m, 2H). MS-ESI: m/z 668.2 observed [M + H]⁺ |
| 106 | 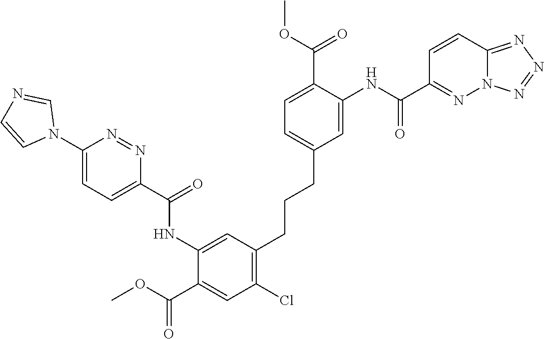 [SMILES: COC(=O)c1cc(Cl)c(CCCc2ccc(C(=O)OC)c(NC(=O)c3ccc4nnnn4n3)c2)cc1NC(=O)c1ccc(-n2ccnc2)nn1] | MS-ESI: m/z 696.6 observed [M + H]⁺ |
| 107 | 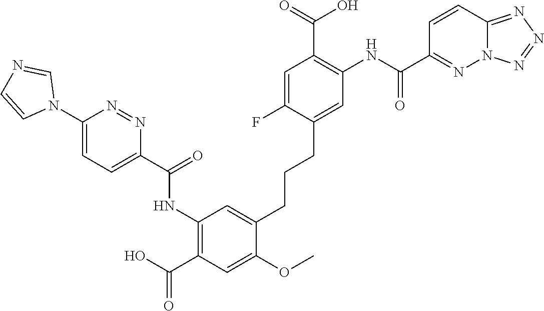 [SMILES: COc1cc(C(=O)O)c(NC(=O)c2ccc(-n3ccnc3)nn2)cc1CCCc1cc(NC(=O)c2ccc3nnnn3n2)c(C(=O)O)cc1F] | MS-ESI: m/z 682.37 observed [M + H]⁺ |
| 108 | 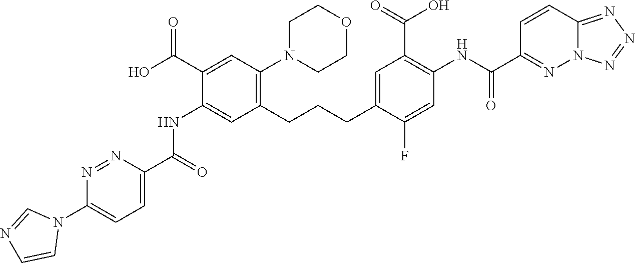 [SMILES: O=C(Nc1cc(CCCc2cc(C(=O)O)c(NC(=O)c3ccc4nnnn4n3)cc2F)c(N2CCOCC2)cc1C(=O)O)c1ccc(-n2ccnc2)nn1] | MS-ESI: m/z 737.22 observed [M + H]⁺ |
| 109 | 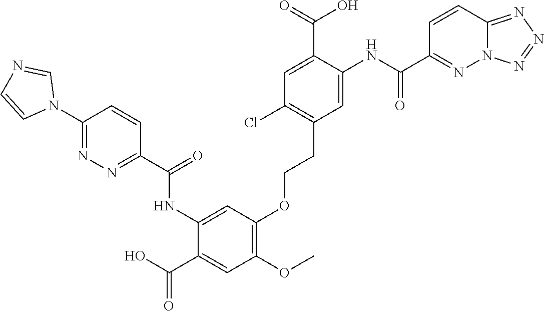 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1Cl] | ¹H NMR (400 MHz, DMSO-d₆) δ 8.92 (d, J = 9.6 Hz, 1H), 8.84 (s, 1H), 8.76 (s, 1H), 8.61 (s, 1H), 8.44-8.32 (m, 3H), 8.17 (s, 1H), 8.07 (s, 1H), 7.63 (s, 1H), 7.25 (s, 1H), 4.27 (t, J = 7.2 Hz, 2H), 3.29- 3.27 (m, 2H). MS-ESI: m/z 700.1 observed [M + H]⁺ |
| 110 | 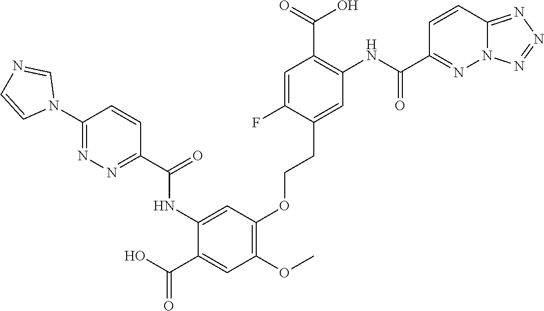 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1F] | ¹H NMR (400 MHz, DMSO-d₆) δ 8.92 (d, J = 9.4 Hz, 1H), 8.78 (d, J = 5.9 Hz, 2H), 8.61 (s, 1H), 8.52- 8.37 (m, 2H), 8.33 (d, J = 9.4 Hz, 1H), 8.18 (s, 1H), 7.74 (d, J = 10.7 Hz, 1H), 7.60 (s, 1H), 7.25 (s, 1H), 4.42-4.17 (m, 2H), 3.76 (s, 3H), 3.22- 3.17 (m, 2H). MS-ESI: m/z 684.17 observed [M + H]⁺ |
| 111 | 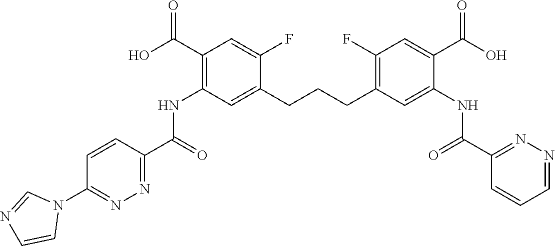 [SMILES: O=C(Nc1cc(CCCc2cc(NC(=O)c3ccc(-n4ccnc4)nn3)c(C(=O)O)cc2F)c(F)cc1C(=O)O)c1cccnn1] | MS-ESI: m/z 629.81 observed [M + H]⁺ |
| 112 | 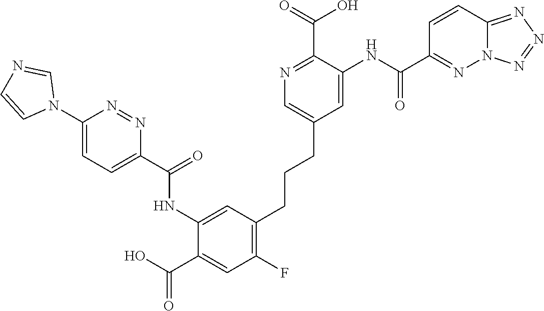 [SMILES: O=C(Nc1cc(CCCc2cnc(C(=O)O)c(NC(=O)c3ccc4nnnn4n3)c2)c(F)cc1C(=O)O)c1ccc(-n2ccnc2)nn1] | ¹H NMR (400 MHz, DMSO-d₆) δ 13.16 (s, 1H), 13.08 (s, 1H), 9.19 (s, 1H), 9.04- 9.00 (m, 2H), 8.82 (d, J = 6.8 Hz, 1H), 8.57 (d, J = 9.2 Hz, 2H), 8.50-8.46 (m, 2H), 8.36-8.33 (m, 2H), 7.74 (d, J = 10.0 Hz, 1H), 7.50 (s, 1H), 2.94-2.84 (m, 4H), 2.11-2.08 (m, 2H). MS-ESI: m/z 653.2 observed [M + H]⁺ |
| 113 | 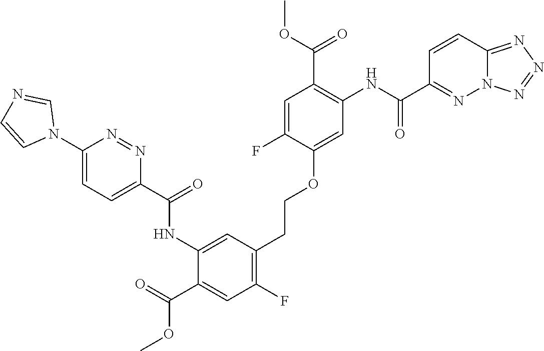 [SMILES: COC(=O)c1cc(F)c(CCOc2cc(NC(=O)c3ccc4nnnn4n3)c(C(=O)OC)cc2F)cc1NC(=O)c1ccc(-n2ccnc2)nn1] | MS-ESI: m/z 700.72 observed [M + H]⁺ |
| 114 | 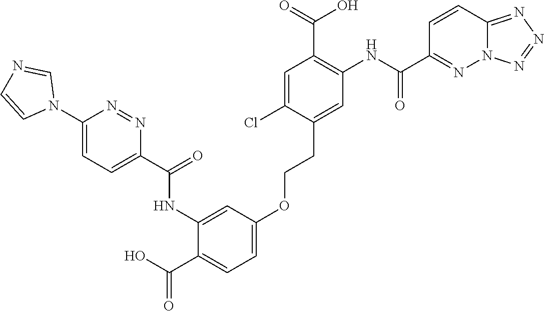 [SMILES: O=C(Nc1cc(OCCc2cc(NC(=O)c3ccc4nnnn4n3)c(C(=O)O)cc2Cl)ccc1C(=O)O)c1ccc(-n2ccnc2)nn1] | ¹H NMR (400 MHz, DMSO-d₆) δ 16.19 (s, 1H), 8.92 (d, J = 9.6 Hz, 1H), 8.84 (s, 1H), 8.77 (s, 1H), 8.42-8.34 (m, 4H), 8.18 (s, 1H), 8.05 (s, 1H), 8.96-8.80 (m, 1H), 7.15 (s, 1H), 6.60 (dd, J = 8.8, 2.0 Hz, 1H), 4.26 (t, J = 6.4 Hz, 2H), 3.23 (t, J = 6.8 Hz, 2H). MS-ESI: m/z 670.1 observed [M + H]⁺ |
| 115 | 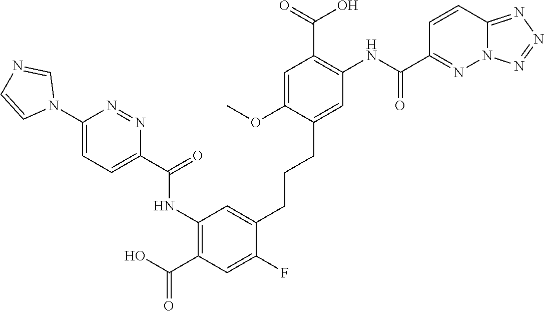 [SMILES: COc1cc(C(=O)O)c(NC(=O)c2ccc3nnnn3n2)cc1CCCc1cc(NC(=O)c2ccc(-n3ccnc3)nn2)c(C(=O)O)cc1F] | MS-ESI: m/z 682.2 observed [M + H]⁺ |
| 116 | 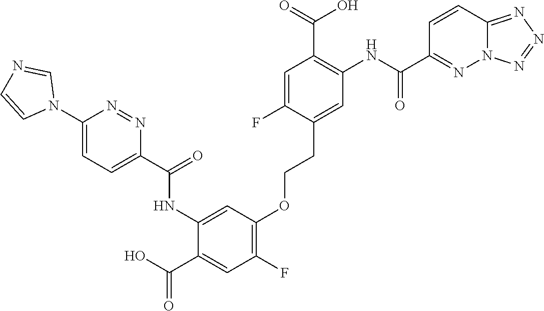 [SMILES: O=C(Nc1cc(OCCc2cc(NC(=O)c3ccc4nnnn4n3)c(C(=O)O)cc2F)c(F)cc1C(=O)O)c1ccc(-n2ccnc2)nn1] | ¹H NMR (400 MHz, DMSO-d₆) δ 8.91 (dd, J = 9.4, 1.4 Hz, 1H), 8.81-8.73 (m, 2H), 8.69 (dd, J = 8.2, 2.1 Hz, 1H), 8.45 (dd, J = 9.1, 0.9 Hz, 1H), 8.39 (dd, J = 9.2, 1.1 Hz, 1H), 8.33 (d, J = 9.5 Hz, 1H), 8.18 (t, J = 1.4 Hz, 1H), 7.79-7.66 (m, 2H), 7.25 (t, J = 1.2 Hz, 1H), 4.32 (t, J = 6.8 Hz, 2H), 3.20 (t, J = 6.8 Hz, 2H), MS-ESI: m/z 670.7 observed [M − H]⁻ |
| 117 | 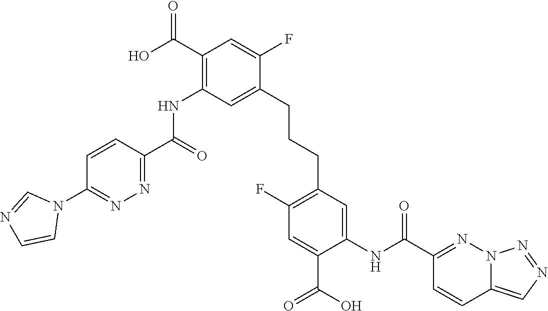 [SMILES: O=C(Nc1cc(CCCc2cc(NC(=O)c3ccc4cnnn4n3)c(C(=O)O)cc2F)c(F)cc1C(=O)O)c1ccc(-n2ccnc2)nn1] | ¹H NMR (500 MHz, DMSO-d₆) δ 8.76 (s, 1H), 8.72 (d, J = 7.2 Hz, 1H), 8.66 (dd, J = 8.2, 3.8 Hz, 2H), 8.47-8.40 (m, 2H), 8.36 (d, J = 9.1 Hz, 1H), 8.18 (s, 1H), 7.90 (d, J = 9.2 Hz, 1H), 7.68 (dd, J = 10.9, 4.2 Hz, 2H), 7.25 (s. 1H), 2.72 (d, J = 8.8 Hz, 5H), 1.23 (s, 6H). MS-ESI: m/z 669.3 observed [M + H]⁺ |
| 118 | 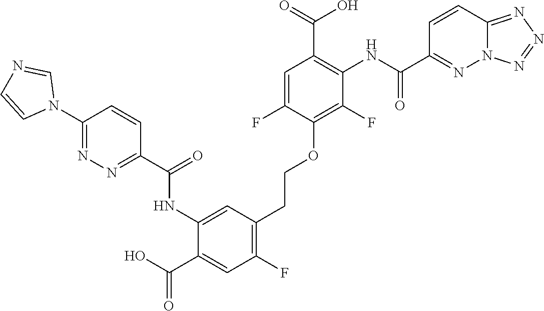 [SMILES: O=C(Nc1cc(CCOc2c(F)cc(C(=O)O)c(NC(=O)c3ccc4nnnn4n3)c2F)c(F)cc1C(=O)O)c1ccc(-n2ccnc2)nn1] | MS-ESI: m/z 690.09 observed [M + H]⁺ |
| 119 | 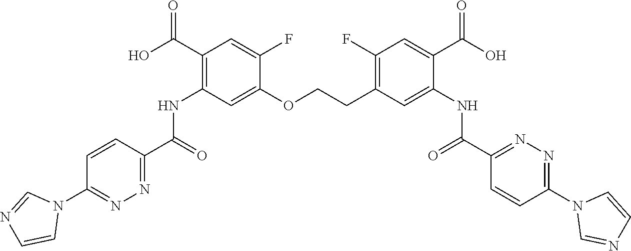 [SMILES: O=C(Nc1cc(CCOc2cc(NC(=O)c3ccc(-n4ccnc4)nn3)c(C(=O)O)cc2F)c(F)cc1C(=O)O)c1ccc(-n2ccnc2)nn1] | ¹H NMR (500 MHz, DMSO-d₆) δ 8.82 (d, J = 7.1 Hz, 1H), 8.77 (d, J = 3.4 Hz, 2H), 8.70 (d, J = 8.1 Hz, 1H), 8.44 (d, J = 9.1 Hz, 2H), 8.38 (dd, J = 9.1, 1.9 Hz, 2H), 8.19 (d, J = 3.7 Hz, 2H), 7.79-7.64 (m, 2H), 7.25 (s, 2H), 4.32 (t, J = 7.0 Hz, 2H), 3.18 (t, J = 6.9 Hz, 2H). MS-ESI: m/z 697.16 observed [M + H]⁺ |
| 120 | 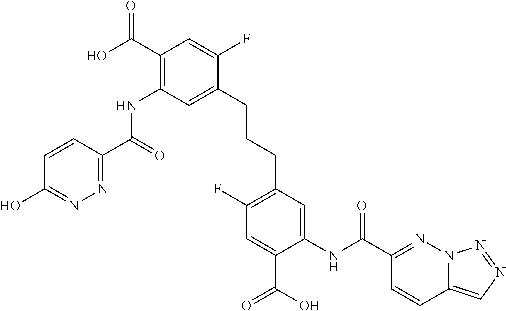 [SMILES: O=C(Nc1cc(CCCc2cc(NC(=O)c3ccc4cnnn4n3)c(C(=O)O)cc2F)c(F)cc1C(=O)O)c1ccc(O)nn1] | ¹H NMR (500 MHz, DMSO-d₆) δ 9.56 (d, J = 7.0 Hz, 1H), 8.85 (s, 1H), 8.70 (d, J = 7.3 Hz, 1H), 8.57 (d, J = 7.3 Hz, 1H), 7.95-7.89 (m, 2H), 7.64 (dd, J = 20.6, 11.0 Hz, 2H), 6.94 (d, J = 9.8 Hz, 1H), 2.69 (q, J = 7.7 Hz, 4H), 2.65-2.62 (m, 3H), 2.37-2.35 (m, 3H), 1.23 (s, 1H). MS-ESI: m/z 619.15 observed [M + H]⁺ |
| 121 | 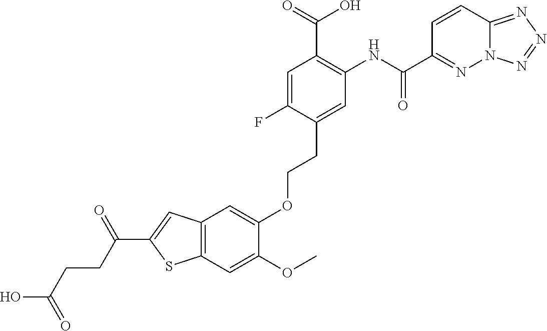 [SMILES: COc1cc2sc(C(=O)CCC(=O)O)cc2cc1OCCc1cc(NC(=O)c2ccc3nnnn3n2)c(C(=O)O)cc1F] | MS-ESI: m/z 609.07 observed [M + H]⁺ |
| 122 | 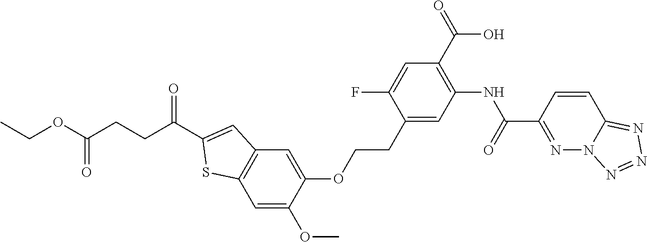 [SMILES: CCOC(=O)CCC(=O)c1cc2cc(OCCc3cc(NC(=O)c4ccc5nnnn5n4)c(C(=O)O)cc3F)c(OC)cc2s1] | MS-ESI: m/z 637.11 observed [M + H]⁺ |
| 123 | 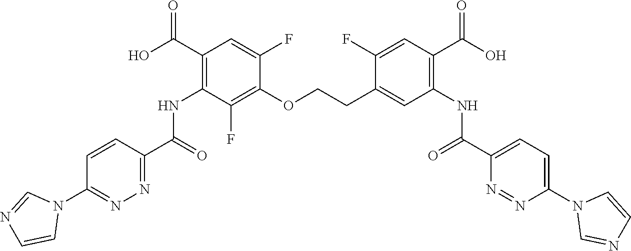 [SMILES: O=C(Nc1cc(CCOc2c(F)cc(C(=O)O)c(NC(=O)c3ccc(-n4ccnc4)nn3)c2F)c(F)cc1C(=O)O)c1ccc(-n2ccnc2)nn1] | ¹H NMR (400 MHz, DMSO-d₆) δ 13.86 (s, 1H), 8.79 (t, J = 5.9 Hz, 3H), 8.46 (dd, J = 9.1, 2.5 Hz, 2H), 8.40 (dd, J = 9.2, 5.3 Hz, 2H), 8.20 (dt, J = 4.3, 1.5 Hz, 2H), 7.70 (d, J = 11.0 Hz, 1H), 7.48 (d, J = 12.0 Hz, 1H), 7.26- 7.22 (m, 2H), 4.37 (t, J = 7.1 Hz, 2H), 3.12 (t, J = 7.1 Hz, 2H), 1.23 (s, 2H). MS-ESI: m/z 715.16 observed [M + H]⁺ |
| 124 | 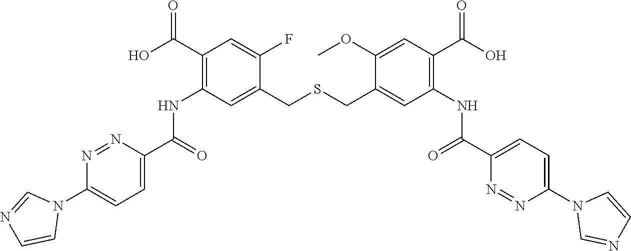 [SMILES: COc1cc(C(=O)O)c(NC(=O)c2ccc(-n3ccnc3)nn2)cc1CSCc1cc(NC(=O)c2ccc(-n3ccnc3)nn2)c(C(=O)O)cc1F] | ¹H NMR (400 MHz, DMSO-d₆) δ 15.76 (s, 1H), 15.67 (s, 1H), 8.85-8.76 (m, 4H), 8.45-8.39 (m, 4H), 8.19 (s, 2H), 7.75-7.69 (m, 2H), 7.26 (s, 2H), 3.90-3.70 (m, 7H). MS-ESI: m/z 725.4 observed [M + H]⁺ |
| 125 | 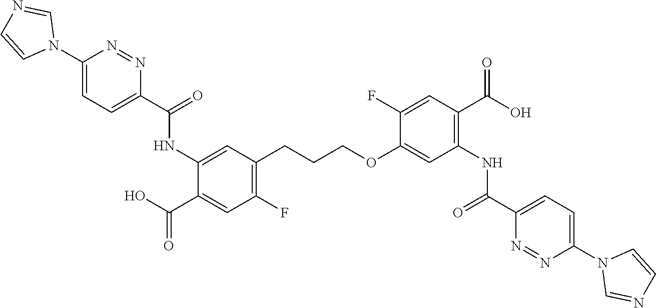 [SMILES: O=C(Nc1cc(CCCOc2cc(NC(=O)c3ccc(-n4ccnc4)nn3)c(C(=O)O)cc2F)c(F)cc1C(=O)O)c1ccc(-n2ccnc2)nn1] | ¹H NMR (500 MHz, DMSO-d₆) δ 8.76 (d, J = 5.4 Hz, 3H), 8.66 (d, J = 8.1 Hz, 1H), 8.43 (dd, J = 9.1, 4.3 Hz, 2H), 8.37 (d, J = 9.0 Hz, 2H), 8.17 (s, 2H), 7.76 (d, J = 12.5 Hz, 1H), 7.70 (d, J = 10.9 Hz, 1H), 7.24 (s, 2H), 4.16 (t, J = 6.3 Hz, 2H), 2.84 (t, J = 7.8 Hz, 2H), 2.12 (t, J = 7.6 Hz, 2H). MS-ESI: m/z 711.4 observed [M + H]⁺ |
| 126 | 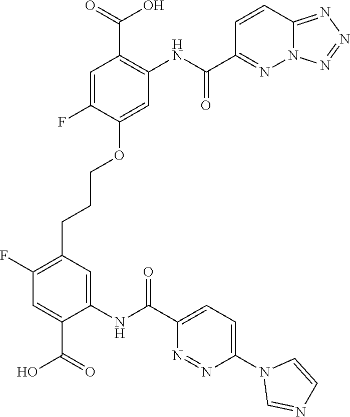 [SMILES: O=C(Nc1cc(CCCOc2cc(NC(=O)c3ccc4nnnn4n3)c(C(=O)O)cc2F)c(F)cc1C(=O)O)c1ccc(-n2ccnc2)nn1] | ¹H NMR (500 MHz, DMSO-d₆) δ 8.93 (d, J = 9.5 Hz, 1H), 8.83- 8.73 (m, 2H), 8.59 (d, J = 8.0 Hz, 1H), 8.45 (d, J = 9.0 Hz, 1H), 8.39 (d, J = 9.2 Hz, 1H), 8.34 (d, J = 9.4 Hz, 1H), 8.17 (s, 1H), 7.75 (dd, J = 27.0, 11.6 Hz, 2H), 7.25 (s, 1H), 4.20 (t, J = 6.2 Hz, 2H), 2.88 (t, J = 7.9 Hz, 2H), 2.15 (t, J = 7.5 Hz, 2H). MS-ESI: m/z 686.5 observed [M + H]⁺ |
| 127 | 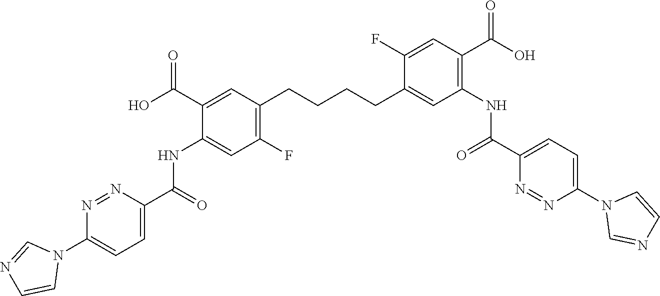 [SMILES: O=C(Nc1cc(CCCCc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 15.44 (s, 2H), 8.78 (s, 2H), 8.72-8.54 (m, 2H), 8.51-8.39 (m, 4H), 8.19 (s, 2H), 7.99-7.93 (m, 2H), 7.26 (s, 2H), 1.76-1.66 (m, 5H), 1.24 (s, 1H). MS-ESI: m/z 709.2 observed [M + H]⁺ |
| 128 | 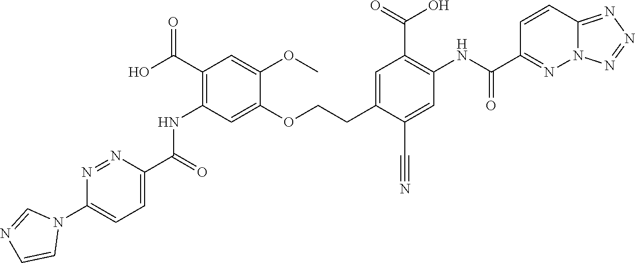 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1C#N] | ¹H NMR (500 MHz, DMSO-d₆) δ 9.03 9.01 (m, 1H), 8.94 (d, J = 9.5 Hz, 1H), 8.78 (s, 1H), 8.62 (s, 1H), 8.51 (d, J = 9.1 Hz, 1H), 8.43 (d, J = 9.2 Hz, 1H), 8.35 (d, J = 9.5 Hz, 1H), 8.27 (s, 1H), 8.19 (s, 1H), 7.58 (s, 1H), 7.27-7.10 (m, 4H), 6.58 (s, 1H), 4.32 (t, J = 7.2 Hz, 2H), 3.77 (s, 3H), 1.23 (s, 1H). MS-ESI: m/z 691.4 observed [M + H]⁺ |
| 129 | 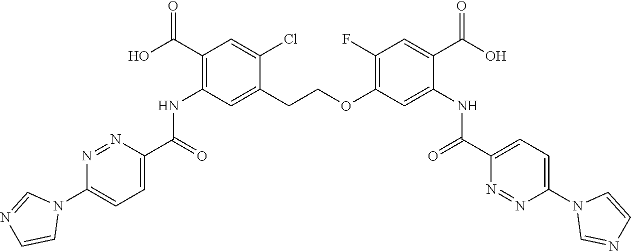 [SMILES: O=C(Nc1cc(OCCc2cc(NC(=O)c3ccc(-n4ccnc4)nn3)c(C(=O)O)cc2Cl)c(F)cc1C(=O)O)c1ccc(-n2ccnc2)nn1] | ¹H NMR (500 MHz, DMSO-d₆) δ 8.88 (s, 1H), 8.77 (d, J = 6.0 Hz, 2H), 8.70 (d, J = 8.1 Hz, 1H), 8.44 (dd, J = 9.1, 4.0 Hz, 2H), 8.38 (d, J = 9.1 Hz, 2H), 8.18 (d, J = 6.1 Hz, 2H), 8.04 (s, 1H), 7.73 (d, J = 12.5 Hz, 1H), 7.25 (d, J = 3.0 Hz, 2H), 4.32 (t, J = 7.1 Hz, 2H), 3.27-3.24 (m, 2H). MS-ESI: m/z 713.0 observed [M + H]⁺ |
| 130 | 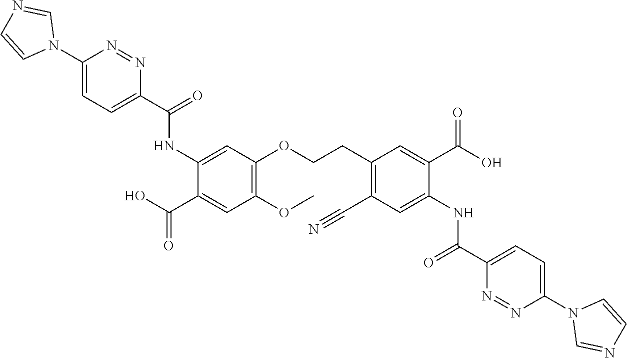 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1C#N] | ¹H NMR (500 MHz, DMSO-d₆) δ ¹H NMR (500 MHz, DMSO) δ 9.09 (s, 1H), 8.78 (d, J = 17.3 Hz, 2H), 8.56 (s, 1H), 8.44 (ddd, J = 35.2, 15.0, 9.1 Hz, 4H), 8.26-8.14 (m, 3H), 7.64 (s, 1H), 7.25 (dd, J = 8.6, 1.4 Hz, 2H), 4.25 (t, J = 7.0 Hz, 2H), 3.74 (s, 3H), 2.53-2.52 (m, 2H). MS-ESI: m/z 716.4 observed [M + H]⁺ |
| 131 | 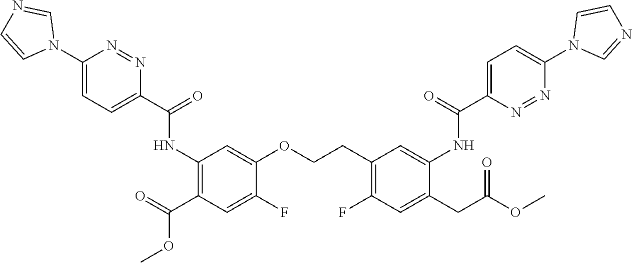 [SMILES: COC(=O)Cc1cc(F)c(CCOc2cc(NC(=O)c3ccc(-n4ccnc4)nn3)c(C(=O)OC)cc2F)cc1NC(=O)c1ccc(-n2ccnc2)nn1] | ¹H NMR (400 MHz, DMSO-d₆) δ 13.12 (s, 1H), 10.74 (s, 1H), 8.81 (d, J= 11.6 Hz, 3H), 8.53-8.45 (m, 4H), 8.21 (d, J = 13.6 Hz, 2H), 7.84 (d, J = 12 Hz, 1H), 7.29 (d, J = 12.8 Hz, 3H), 4.45 (t, J = 6.4 Hz, 2H), 3.92 (s, 3H), 3.81 (s, 2H), 3.53 (s, 3H), 3.25- 3.22 (m, 2H). MS-ESI: m/z 739.5 observed [M + H]⁺ |
| 132 | 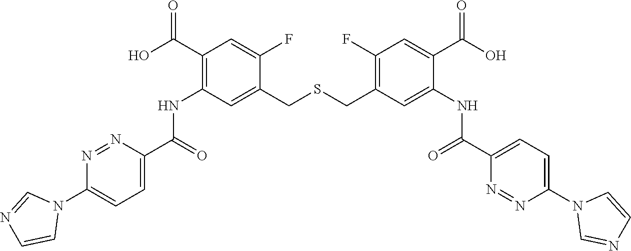 [SMILES: O=C(Nc1cc(CSCc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 8.85 (d, J = 7.1 Hz, 2H), 8.76 (s, 2H), 8.43 (d, J = 9.2 Hz, 2H), 8.36 (d, J = 9.1 Hz, 2H), 8.15 (s, 2H), 7.69 (d, J = 10.5 Hz, 2H), 7.24 (s, 2H), 3.89 (s, 4H). MS-ESI: m/z 713.14 observed [M + H]⁺ |
| 133 | 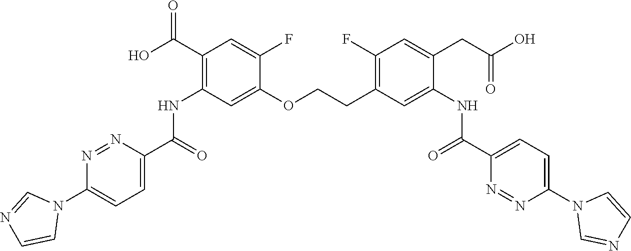 [SMILES: O=C(O)Cc1cc(F)c(CCOc2cc(NC(=O)c3ccc(-n4ccnc4)nn3)c(C(=O)O)cc2F)cc1NC(=O)c1ccc(-n2ccnc2)nn1] | ¹H NMR (400 MHz, DMSO-d₆) δ 10.73 (s, 1H), 8.79-8.77 (m, 3H), 8.51-8.41 (m, 4H), 8.19 (s, 2H), 7.80-7.74 (m, 2H), 7.29-7.27 (m, 3H), 4.39 (t, J = 6.8 Hz, 2H), 3.72 (s, 2H) 3.23- 3.21 (m, 2H). MS-ESI: m/z 711.1 observed [M + H]⁺ |
| 134 | 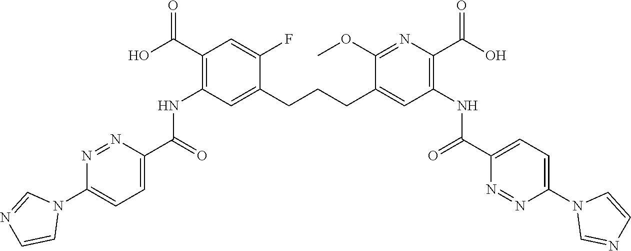 [SMILES: COc1nc(C(=O)O)c(NC(=O)c2ccc(-n3ccnc3)nn2)cc1CCCc1cc(NC(=O)c2ccc(-n3ccnc3)nn2)c(C(=O)O)cc1F] | ¹H NMR (400 MHz, DMSO-d₆) δ 8.97 (s, 1H), 8.77 (s, 2H), 8.72 (d, J = 7.2 Hz, 1H), 8.47-8.31 (m, 4H), 8.18 (s, 2H), 7.68 (d, J = 11.0 Hz, 1H), 7.25 (s, 2H), 3.90 (s, 3H), 2.77- 2.67 (m, 4H), 1.92 (d, J = 8.7 Hz, 2H). MS-ESI: m/z 708.2 observed [M + H]⁺ |
| 135 | 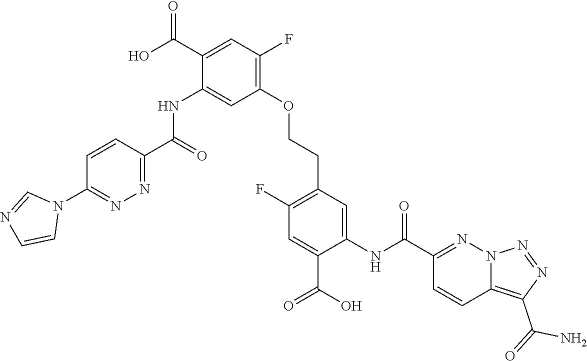 [SMILES: NC(=O)c1nnn2nc(C(=O)Nc3cc(CCOc4cc(NC(=O)c5ccc(-n6ccnc6)nn5)c(C(=O)O)cc4F)c(F)cc3C(=O)O)ccc12] | MS-ESI: m/z 714.31 observed [M + H]⁺ |
| 136 | 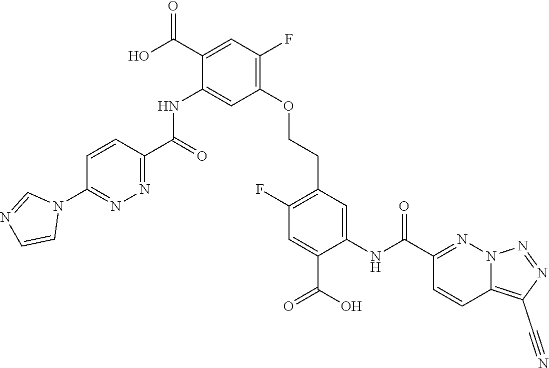 [SMILES: N#Cc1nnn2nc(C(=O)Nc3cc(CCOc4cc(NC(=O)c5ccc(-n6ccnc6)nn5)c(C(=O)O)cc4F)c(F)cc3C(=O)O)ccc12] | MS-ESI: m/z 696.17 observed [M + H]⁺ |
| 137 | 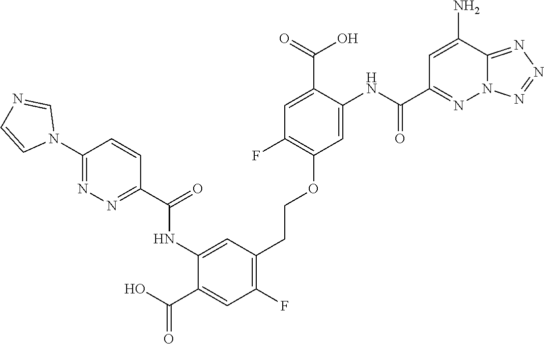 [SMILES: Nc1cc(C(=O)Nc2cc(OCCc3cc(NC(=O)c4ccc(-n5ccnc5)nn4)c(C(=O)O)cc3F)c(F)cc2C(=O)O)nn2nnnc12] | MS-ESI: m/z 687.31 observed [M + H]⁺ |
| 138 | 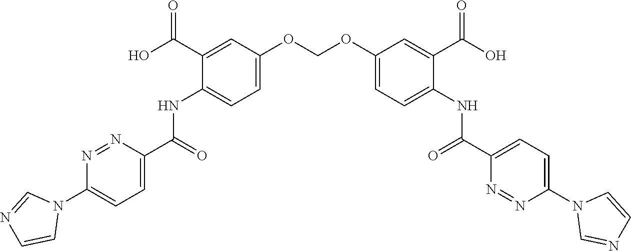 [SMILES: O=C(Nc1ccc(OCOc2ccc(NC(=O)c3ccc(-n4ccnc4)nn3)c(C(=O)O)c2)cc1C(=O)O)c1ccc(-n2ccnc2)nn1] | MS-ESI: m/z 663.1 observed [M + H]⁺ |
| 139 | 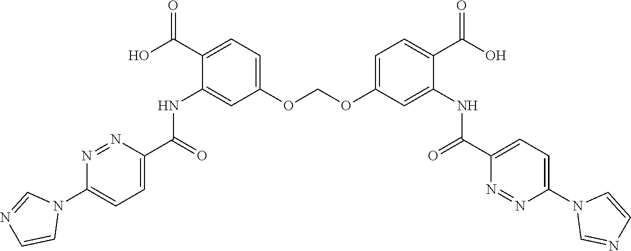 [SMILES: O=C(Nc1cc(OCOc2ccc(C(=O)O)c(NC(=O)c3ccc(-n4ccnc4)nn3)c2)ccc1C(=O)O)c1ccc(-n2ccnc2)nn1] | MS-ESI: m/z 663.2 observed [M + H]⁺ |
| 140 | 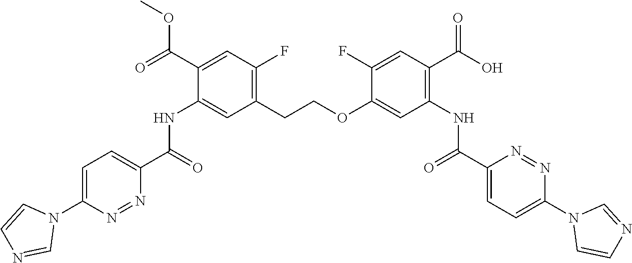 [SMILES: COC(=O)c1cc(F)c(CCOc2cc(NC(=O)c3ccc(-n4ccnc4)nn3)c(C(=O)O)cc2F)cc1NC(=O)c1ccc(-n2ccnc2)nn1] | MS-ESI: m/z 711.79 observed [M + H]⁺ |
| 141 | 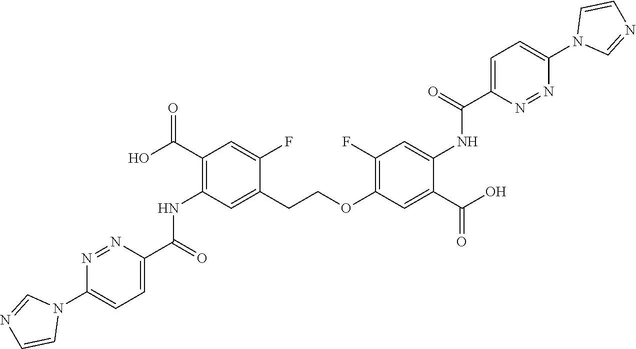 [SMILES: O=C(Nc1cc(CCOc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 9.52 (s, 2H), 8.65 (s, 3H), 8.39 (d, J = 12.4 Hz, 1H), 8.24 (s, 2H), 8.15 (s, 2H), 7.80 (t, J = 9.6 Hz, 2H), 7.58 (s, 2H), 4.35 (s, 2H), 3.19 (s, 2H). MS-ESI: m/z 697.2 observed [M + H]⁺ |
| 142 | 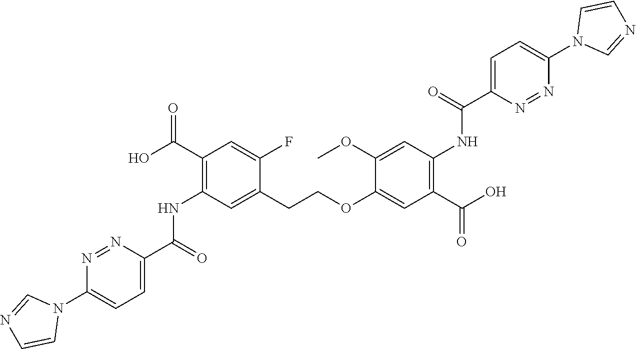 [SMILES: COc1cc(NC(=O)c2ccc(-n3ccnc3)nn2)c(C(=O)O)cc1OCCc1cc(NC(=O)c2ccc(-n3ccnc3)nn2)c(C(=O)O)cc1F] | MS-ESI: m/z 709.2 observed [M + H]⁺ |
| 143 | 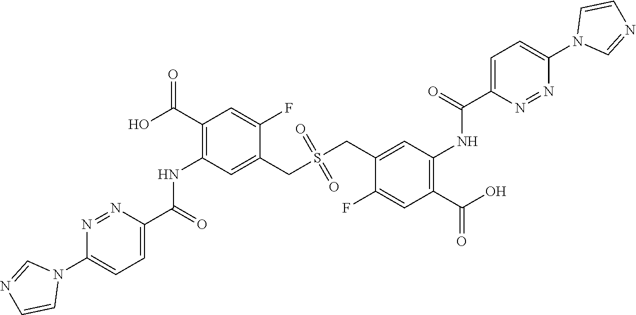 [SMILES: O=C(Nc1cc(CS(=O)(=O)Cc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 15.72 (s, 2H), 8.87-8.82 (m, 2H), 8.77 (s, 2H), 8.48-8.36 (m, 4H), 8.19 (s, 2H), 7.78 (d, J = 12.8 Hz, 2H), 7.25 (s, 2H), 4.38 (d, J = 13.2 Hz, 2H), 4.18 (s, 2H). MS-ESI: m/z 745.14 observed [M + H]⁺ |
| 144 | 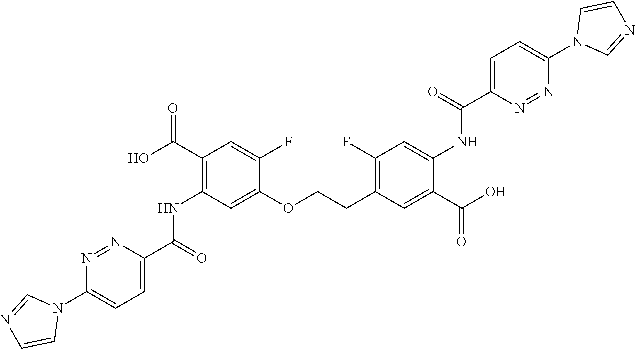 [SMILES: O=C(Nc1cc(OCCc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 8.78 (d, J = 7.6 Hz, 2H), 8.69 (d, J = 8.1 Hz, 1H), 8.54 (d, J = 12.9 Hz, 1H), 8.47 (d, J = 9.2 Hz, 2H), 8.44-8.33 (m, 2H), 8.20 (d, J = 7.5 Hz, 2H), 8.10 (d, J = 9.5 Hz, 1H), 7.72 (d, J = 12.6 Hz, 1H), 7.25 (d, J = 4.6 Hz, 2H), 4.27 (t. J = 7.0 Hz, 2H), 3.15 (d, J = 7.3 Hz, 2H). MS-ESI: m/z 697.16 observed [M + H]⁺ |
| 145 | 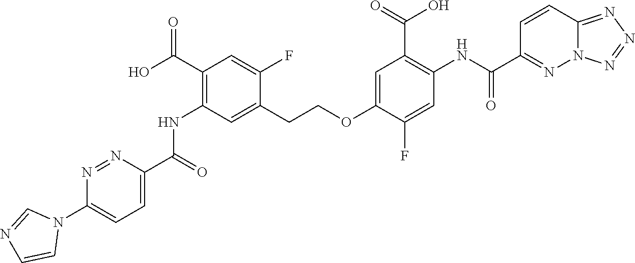 [SMILES: O=C(Nc1cc(CCOc2cc(C(=O)O)c(NC(=O)c3ccc4nnnn4n3)cc2F)c(F)cc1C(=O)O)c1ccc(-n2ccnc2)nn1] | MS-ESI: m/z 672.2 observed [M + H]⁺ |
| 146 | 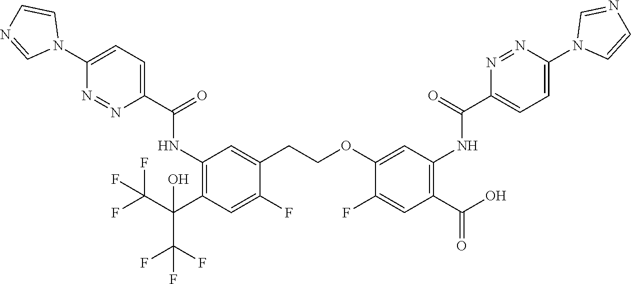 [SMILES: O=C(Nc1cc(OCCc2cc(NC(=O)c3ccc(-n4ccnc4)nn3)c(C(O)(C(F)(F)F)C(F)(F)F)cc2F)c(F)cc1C(=O)O)c1ccc(-n2ccnc2)nn1] | ¹H NMR (400 MHz, DMSO-d₆) δ 8.98 (d, J = 8.2 Hz, 1H), 8.81- 8.68 (m, 3H), 8.48 (d, J = 9.1 Hz, 1H), 8.39 (d, J = 9.2 Hz, 1H), 8.36-8.23 (m, 2H), 8.17 (dt, J = 14.4, 1.4 Hz, 2H), 7.73 (d, J = 12.6 Hz, 1H), 7.24 (dt, J = 7.3, 1.2 Hz, 2H), 7.12 (d, J = 11.6 Hz, 1H), 4.31 (t. J = 7.1 Hz, 2H), 3.14 (t. J = 7.0 Hz, 2H). MS-ESI: m/z 723.5 observed [M + H]⁺ |
| 147 | 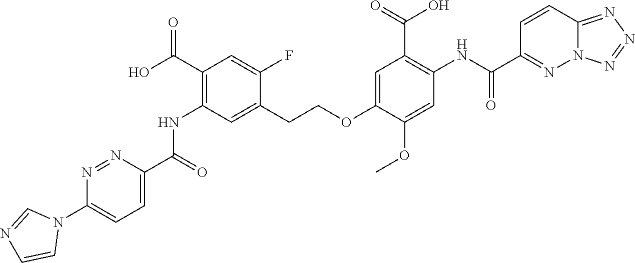 [SMILES: COc1cc(NC(=O)c2ccc3nnnn3n2)c(C(=O)O)cc1OCCc1cc(NC(=O)c2ccc(-n3ccnc3)nn2)c(C(=O)O)cc1F] | ¹H NMR (400 MHz, DMSO-d₆) δ 15.40 (s, 1H), 8.96 (d, J = 9.6 Hz, 1H), 8.85- 8.79 (m, 2H), 7.49 (d, J = 6.4 Hz, 2H), 8.41 (d, J = 9.2 Hz, 1H), 8.36 (d, J = 9.2 Hz, 1H), 8.20 (s, 1H), 7.73 (d, J = 10.8 Hz, 1H), 7.67 (s, 1H), 7.31-7.26 (m, 4H), 4.27-4.24 (m, 2H), 3.82 (s. 3H), 3.15-3.11 (m, 2H). MS-ESI: m/z 684.2 observed [M + H]⁺ |
| 148 | 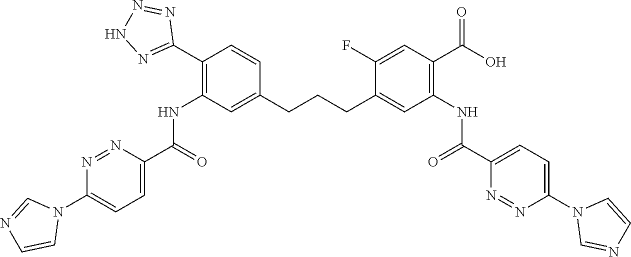 [SMILES: O=C(Nc1cc(CCCc2ccc(-c3nn[nH]n3)c(NC(=O)c3ccc(-n4ccnc4)nn3)c2)c(F)cc1C(=O)O)c1ccc(-n2ccnc2)nn1] | MS-ESI: m/z 701.3 observed [M + H]⁺ |
| 149 | 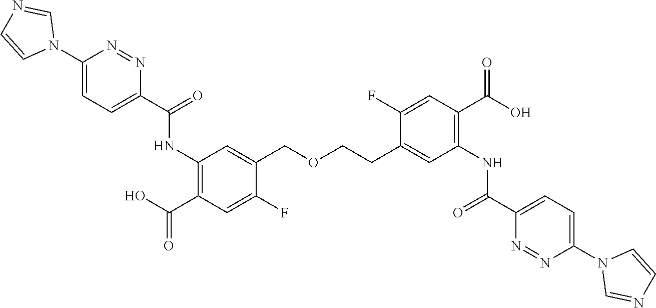 [SMILES: O=C(Nc1cc(CCOCc2cc(NC(=O)c3ccc(-n4ccnc4)nn3)c(C(=O)O)cc2F)c(F)cc1C(=O)O)c1ccc(-n2ccnc2)nn1] | MS-ESI: m/z 711.2 observed [M + H]⁺ |
| 150 | 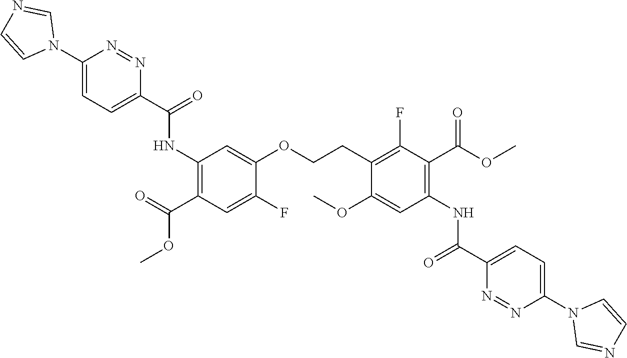 [SMILES: COC(=O)c1cc(F)c(OCCc2c(OC)cc(NC(=O)c3ccc(-n4ccnc4)nn3)c(C(=O)OC)c2F)cc1NC(=O)c1ccc(-n2ccnc2)nn1] | ¹H NMR (400 MHz, DMSO-d₆) δ 13.15 (s, 1H), 12.78 (s, 1H), 9.97 (d, J = 4 Hz, 1H), 8.72-8.60 (m, 8H), 8.31 (s, 1H), 7.89-7.81 (m, 3H), 4.40 (d, J = 6.4 Hz, 2H), 3.95-3.91 (m, 9H), 3.17 (s, 2H). MS-ESI: m/z 755.2 observed [M + H]⁺ |
| 151 | 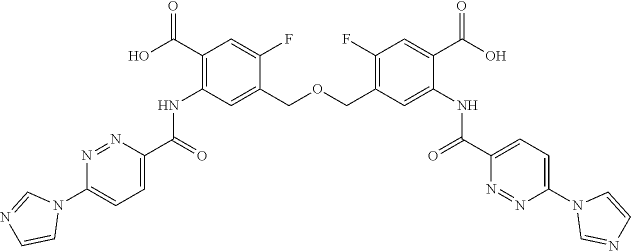 [SMILES: O=C(Nc1cc(COCc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 14.65- 14.58 (m, 2H), 8.96 (d, J = 6.8 Hz, 2H), 8.77 (s, 2H), 8.44- 8.35 (m, 4H), 8.17 (s, 2H), 7.77 (d, J = 10.4 Hz, 2H), 7.24 (s, 4H), 4.73 (s, 4H). MS-ESI: m/z 697.3 observed [M + H]⁺ |
| 152 | 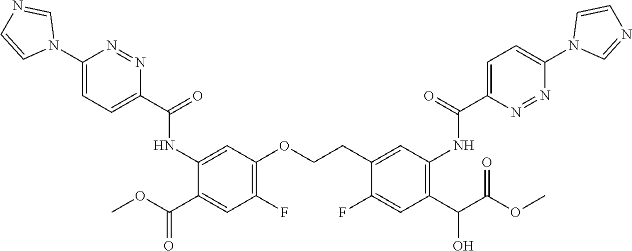 [SMILES: COC(=O)c1cc(F)c(OCCc2cc(NC(=O)c3ccc(-n4ccnc4)nn3)c(C(O)C(=O)OC)cc2F)cc1NC(=O)c1ccc(-n2ccnc2)nn1] | ¹H NMR (400 MHz, DMSO-d₆) δ 11.05 (s, 1H), 8.76 (d, J = 12 Hz, 1H), 8.59 (d, J = 9.2 Hz, 1H), 8.45- 8.38 (m, 3H), 8.17- 8.12 (m, 2H), 7.84- 7.81 (m, 1H), 7.60- 7.57 (m, 1H), 7.36 (dd, J = 12.8 Hz, 1H), 7.24 (d, J = 5.2 Hz, 2H), 6.70 (s, 1H), 6.59-6.52 (m, 3H), 4.29-4.22 (m, 3H), 3.73 (s, 3H), 3.67 (s, 3H), 3.21 (d, J = 8 Hz, 2H). MS-ESI: m/z 755.4 observed [M + H]⁺ |
| 153 | 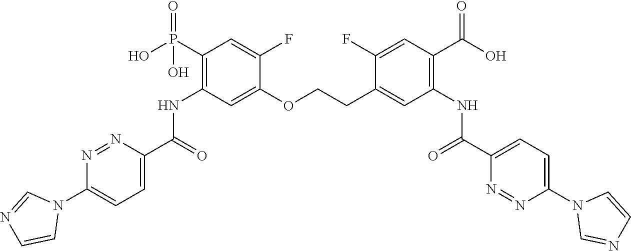 [SMILES: O=C(Nc1cc(CCOc2cc(NC(=O)c3ccc(-n4ccnc4)nn3)c(P(=O)(O)O)cc2F)c(F)cc1C(=O)O)c1ccc(-n2ccnc2)nn1] | MS-ESI: m/z 733.2 observed [M + H]⁺ |
| 154 | 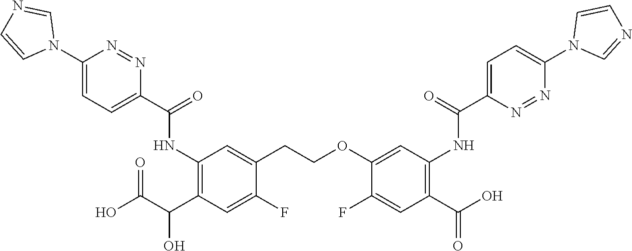 [SMILES: O=C(Nc1cc(OCCc2cc(NC(=O)c3ccc(-n4ccnc4)nn3)c(C(O)C(=O)O)cc2F)c(F)cc1C(=O)O)c1ccc(-n2ccnc2)nn1] | ¹H NMR (400 MHz, DMSO-d₆) δ 15.96 (d, J = 3.6 Hz 1H), 8.79-8.77 (m, 3H), 8.47-8.38 (m, 4H), 8.20-7.28 (m, 4H), 7.26 (d, J = 10 Hz, 3H), 4.78-4.76 (m, 1H), 4.28-4.25 (m, 2H), 3.33-3.15 (m, 2H). MS-ESI: m/z 727.4 observed [M + H]⁺ |
| 155 | 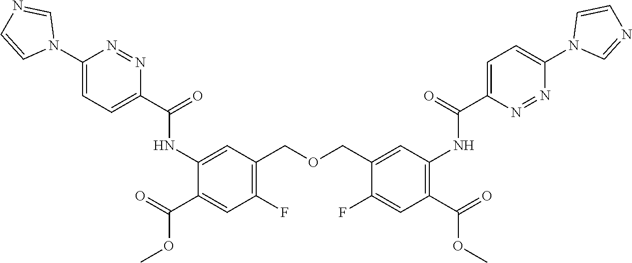 [SMILES: COC(=O)c1cc(F)c(COCc2cc(NC(=O)c3ccc(-n4ccnc4)nn3)c(C(=O)OC)cc2F)cc1NC(=O)c1ccc(-n2ccnc2)nn1] | ¹H NMR (400 MHz, DMSO-d₆) δ 12.85 (s, 2H), 8.97 (d, J = 6.4 Hz, 2H), 8.77 (s, 2H), 8.40 (dd, J = 18, 9.2 Hz, 4H), 8.17 (d, J = 1.2 Hz, 2H), 7.81 (d, J = 10 Hz, 2H), 7.24 (s, 2H), 4.83 (s, 4H), 3.95 (s, 6H). MS-ESI: m/z 725.0 observed [M + H]⁺ |
| 156 | 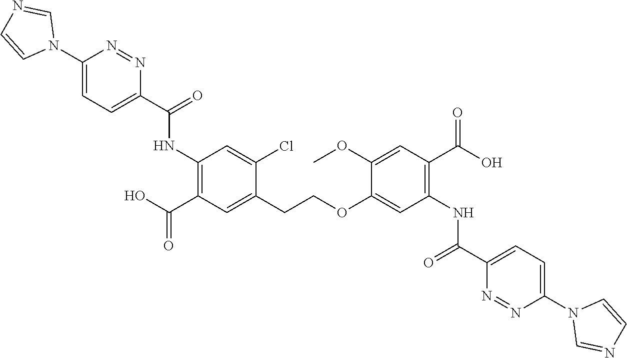 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1Cl] | ¹H NMR (400 MHz, DMSO-d₆) δ 16.13 (s, 1H), 16.01 (s, 1H), 8.87 (s, 1H), 8.58- 8.46 (m, 3H), 8.44- 8.13 (m, 7H), 7.65 (s, 1H), 7.25 (d, J = 7.6 Hz, 2H), 4.20-4.18 (m, 2H), 3.76 (s, 3H), 3.38-3.19 (m, 2H). MS-ESI: m/z 725.2 observed [M + H]⁺ |
| 157 | 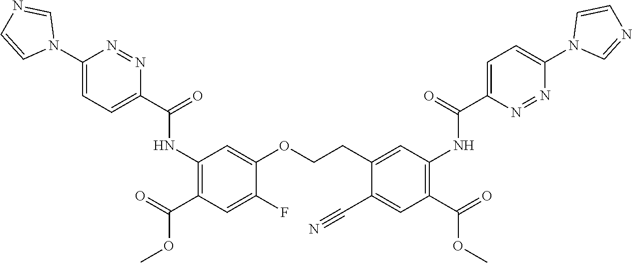 [SMILES: COC(=O)c1cc(F)c(OCCc2cc(NC(=O)c3ccc(-n4ccnc4)nn3)c(C(=O)OC)cc2C#N)cc1NC(=O)c1ccc(-n2ccnc2)nn1] | MS-ESI: m/z 732.3 observed [M + H]⁺ |
| 158 | 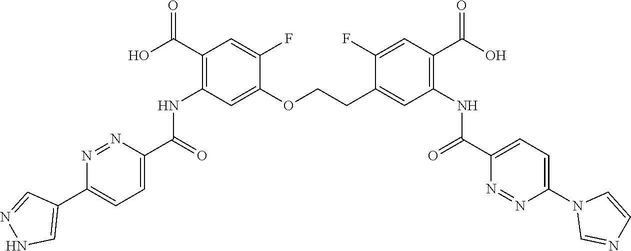 [SMILES: O=C(Nc1cc(OCCc2cc(NC(=O)c3ccc(-n4ccnc4)nn3)c(C(=O)O)cc2F)c(F)cc1C(=O)O)c1ccc(-c2cn[nH]c2)nn1] | MS-ESI: m/z 697.29 observed [M + H]⁺ |
| 159 | 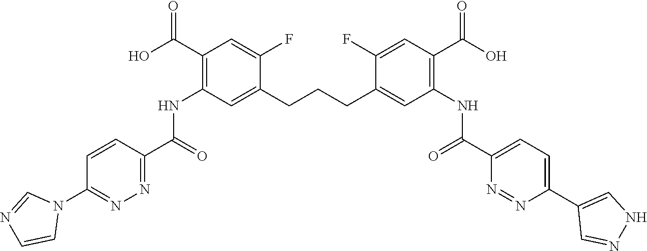 [SMILES: O=C(Nc1cc(CCCc2cc(NC(=O)c3ccc(-n4ccnc4)nn3)c(C(=O)O)cc2F)c(F)cc1C(=O)O)c1ccc(-c2cn[nH]c2)nn1] | ¹H NMR (400 MHz, DMSO-d₆) δ 15.84 (s, 1H), 15.52 (s, 1H), 8.79 (s, 1H), 8.75 (d, J = 7.2 Hz, 2H), 8.45 (d. J = 9.2 Hz, 1H), 8.39 (d, J = 9.2, 2H), 8.20 (s, 1H), 8.11-8.05 (m, 2H), 7.69 (dd, J = 10.8, 2.4 Hz, 2H), 7.27 (s, 1H), 2.74-2.69 (m, 2H), 1.96-1.94 (m, 2H). MS-ESI: m/z 695.1 observed [M + H]⁺ |
| 160 | 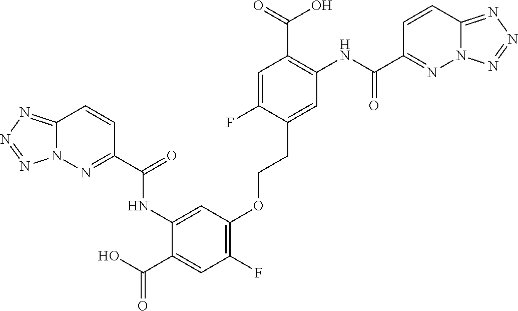 [SMILES: O=C(Nc1cc(CCOc2cc(NC(=O)c3ccc4nnnn4n3)c(C(=O)O)cc2F)c(F)cc1C(=O)O)c1ccc2nnnn2n1] | MS-ESI: m/z 647.31 observed [M + H]⁺ |
| 161 | 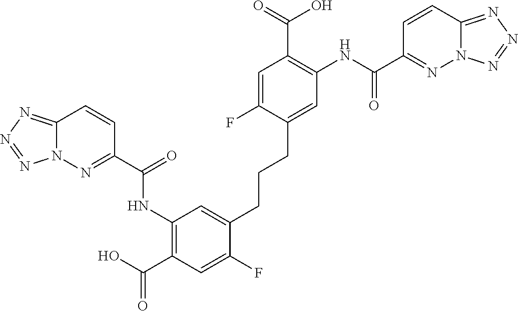 [SMILES: O=C(Nc1cc(CCCc2cc(NC(=O)c3ccc4nnnn4n3)c(C(=O)O)cc2F)c(F)cc1C(=O)O)c1ccc2nnnn2n1] | MS-ESI: m/z 645.14 observed [M + H]⁺ |
| 162 | 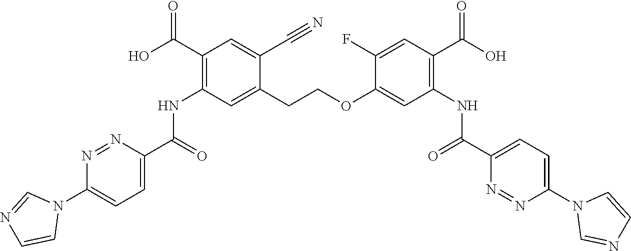 [SMILES: N#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] | ¹H NMR (400 MHz, DMSO-d₆) δ 15.84 (s, 1H), 16.25 (s, 1H), 16.02 (s, 1H), 8.97 (s, 1H), 8.78-8.77 (m, 3H), 8.45-8.35 (m, 5H), 8.19 (d, J = 5.6 Hz, 1H), 7.73 (d, J = 12.4 Hz, 1H), 7.25 (s, 2H), 4.39 (s, 2H), 3.34 (s, 2H). MS-ESI: m/z 704.2 observed [M + H]⁺ |
| 163 | 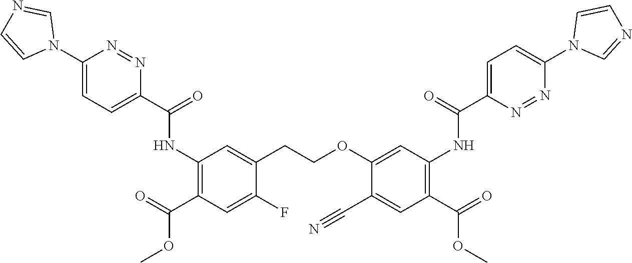 [SMILES: COC(=O)c1cc(F)c(CCOc2cc(NC(=O)c3ccc(-n4ccnc4)nn3)c(C(=O)OC)cc2C#N)cc1NC(=O)c1ccc(-n2ccnc2)nn1] | ¹H NMR (400 MHz, DMSO-d₆) δ 13.43 (s, 1H), 12.94 (s, 1H), 10.29 (d, J = 4.4 Hz, 2H), 8.95 (d, J = 6.8 Hz, 1H), 8.75- 8.64 (m, 7H), 8.28 (s, 1H), 7.95 (s, 2H), 7.74 (d, J = 10 Hz, 1H), 4.54 (t, J = 6 Hz, 2H), 3.88 (d, J = 6.4 Hz, 6H), 3.34-3.31 (m, 2H). MS-ESI: m/z 732.0 observed [M + H]⁺ |
| 164 | 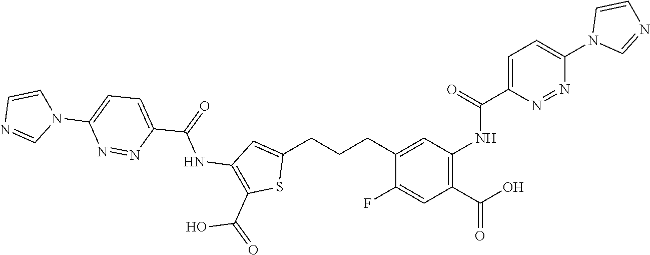 [SMILES: O=C(Nc1cc(CCCc2cc(NC(=O)c3ccc(-n4ccnc4)nn3)c(C(=O)O)s2)c(F)cc1C(=O)O)c1ccc(-n2ccnc2)nn1] | ¹H NMR (400 MHz, DMSO-d₆) δ 15.80 (s, 1H), 13.97 (s, 1H), 8.79-8.78 (m, 3H), 8.46-8.40 (m, 2H), 8.20 (d, J = 1.2 Hz, 2H), 7.85-7.60 (m, 3H), 7.26 (s, 2H), 2.80-2.71 (m, 4H), 2.09-2.07 (m, 2H). MS-ESI: m/z 683.1 observed [M + H]⁺ |
| 165 | 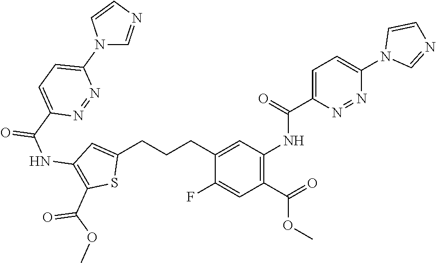 [SMILES: COC(=O)c1cc(F)c(CCCc2cc(NC(=O)c3ccc(-n4ccnc4)nn3)c(C(=O)OC)s2)cc1NC(=O)c1ccc(-n2ccnc2)nn1] | ¹H NMR (400 MHz, DMSO-d₆) δ 12.97 (s, 1H), 12.37 (s, 1H), 10.22 (d, J = 4 Hz, 2H), 8.81 (d, J = 7.2 Hz, 1H), 8.74-8.64 (m, 6H), 8.05 (s, 1H), 8.00 (s, 2H), 7.76 (d, J = 10 Hz, 1H), 3.91 (s, 3H), 3.86 (s, 3H), 3.04 (t. J = 7.2 Hz, 2H), 2.85 (t, J = 7.2 Hz, 2H), 2.07 (t, J = 7.2 Hz, 2H). MS-ESI: m/z 711.2 observed [M + H]⁺ |
| 166 | 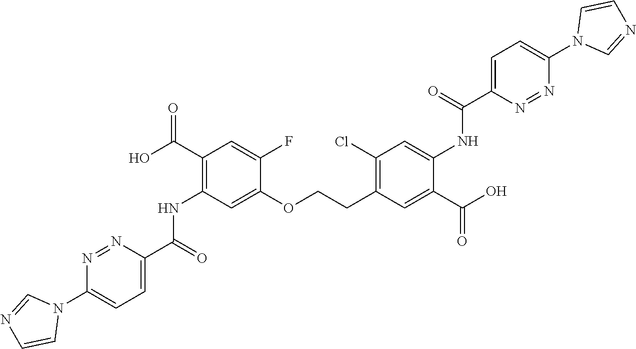 [SMILES: O=C(Nc1cc(OCCc2cc(C(=O)O)c(NC(=O)c3ccc(-n4ccnc4)nn3)cc2Cl)c(F)cc1C(=O)O)c1ccc(-n2ccnc2)nn1] | ¹H NMR (400 MHz, DMSO-d₆) δ 16.03 (s, 1H), 15.99 (s, 1H), 8.88 (s, 1H), 8.80- 8.78 (m, 3H), 8.49- 8.42 (m, 4H), 8.21- 8.15 (m, 3H), 7.77 (d, J = 12.4 Hz, 1H), 7.26 (d, J = 6.4 Hz, 2H), 4.30 (t, J = 7.2 Hz, 2H), 3.25 (t, J = 7.2 Hz, 2H). MS-ESI: m/z 713.1 observed [M + H]⁺ |
| 167 | 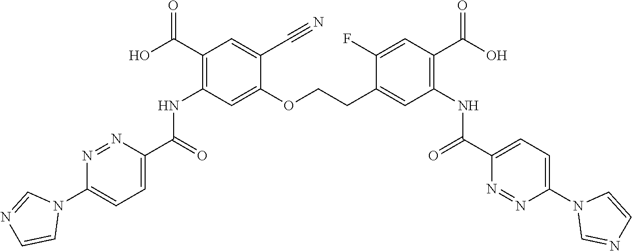 [SMILES: N#Cc1cc(C(=O)O)c(NC(=O)c2ccc(-n3ccnc3)nn2)cc1OCCc1cc(NC(=O)c2ccc(-n3ccnc3)nn2)c(C(=O)O)cc1F] | ¹H NMR (400 MHz, DMSO-d₆) δ 15.76 (s, 1H), 8.84-8.8.71 (m, 4H), 8.46-8.40 (m, 4H), 8.25-8.19 (m, 3H), 7.75 (d, J = 10.8 Hz, 1H), 7.25 (s, 2H), 4.41-4.38 (m, 2H), 3.22 (t, J = 6.8 Hz, 2H). MS-ESI: m/z 704.3 observed [M + H]⁺ |
| 168 | 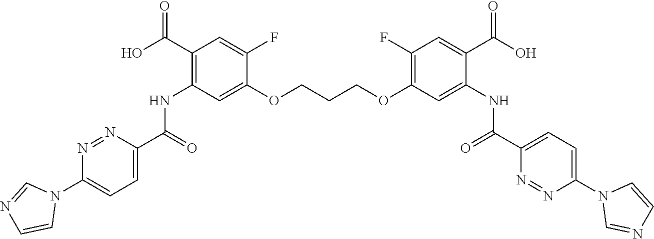 [SMILES: O=C(Nc1cc(OCCCOc2cc(NC(=O)c3ccc(-n4ccnc4)nn3)c(C(=O)O)cc2F)c(F)cc1C(=O)O)c1ccc(-n2ccnc2)nn1] | ¹H NMR (500 MHz, DMSO-d₆) δ 8.77 (d, J = 1.1 Hz, 2H), 8.71 (d, J = 8.3 Hz, 2H), 8.46 (d, J = 9.2 Hz, 2H), 8.40 (d, J = 9.2 Hz, 2H), 8.19 (t, J = 1.4 Hz, 2H), 7.74 (d, J = 12.6 Hz, 2H), 7.25 (t, J = 1.2 Hz, 2H), 4.29 (t, J = 6.3 Hz, 4H), 2.35-2.32 (m, 2H). MS-ESI: m/z 727.17 observed [M + H]⁺ |
| 169 | 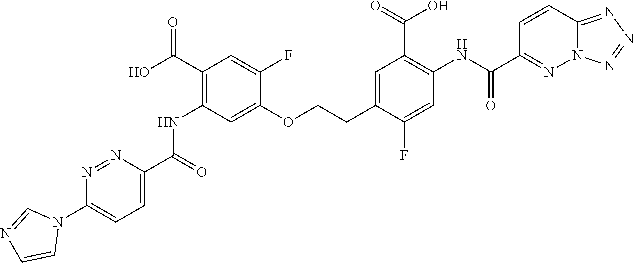 [SMILES: O=C(Nc1cc(OCCc2cc(C(=O)O)c(NC(=O)c3ccc4nnnn4n3)cc2F)c(F)cc1C(=O)O)c1ccc(-n2ccnc2)nn1] | ¹H NMR (400 MHz, DMSO-d₆) δ 16.97 (s, 1H), 16.06 (s, 1H), 8.95 (d, J = 9.6 Hz, 1H), 8.80 (s, 1H), 8.72 (s, 2H), 8.52-8.48 (m, 2H), 8.43-8.35 (m, 2H), 8.21 (s, 1H), 8.14 (s, 2H), 7.76- 7.73 (m, 3H), 7.26 (s, 1H), 4.29-4.22 (m, 2H), 3.16-3.07 (m, 3H). MS-ESI: m/z 672.2 observed [M + H]⁺ |
| 170 | 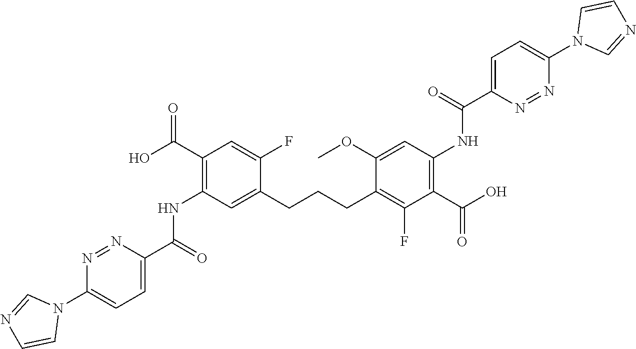 [SMILES: COc1cc(NC(=O)c2ccc(-n3ccnc3)nn2)c(C(=O)O)c(F)c1CCCc1cc(NC(=O)c2ccc(-n3ccnc3)nn2)c(C(=O)O)cc1F] | ¹H NMR (400 MHz, DMSO-d₆) δ 15.77 (s, 1H), 14.69 (s, 1H), 8.78-8.77 (m, 3H), 8.8.46-8.40 (m, 4H), 8.22-8.19 (m, 3H), 7.67 (d, J = 11.2, 1H), 7.25 (d, J = 1.2 Hz, 2H), 3.83 (s, 2H), 2.64 (t, J = 7.2 Hz, 4H) 1.81-1.79 (m, 2H). MS-ESI: m/z 725.2 observed [M + H]⁺ |
| 171 | 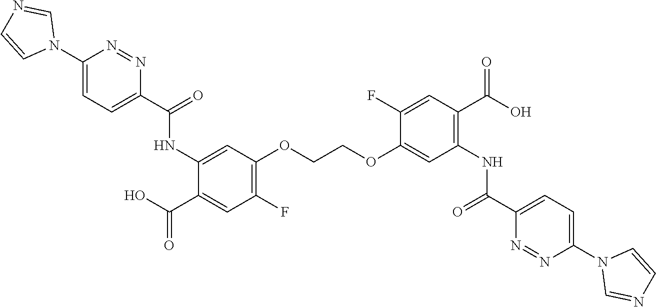 [SMILES: O=C(Nc1cc(OCCOc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 16.19- 16.03 (m, 2H), 8.80- 8.74 (m, 4H), 8.45 (dd, J = 25.6, 9.2 Hz, 5H), 8.21 (s, 2H), 7.77 (d, J = 12.4 Hz, 2H), 7.27 (s, 2H), 7.14 (s, 2H), 4.50 (s, 4H). MS-ESI: m/z 713.2 observed [M + H]⁺ |
| 172 | 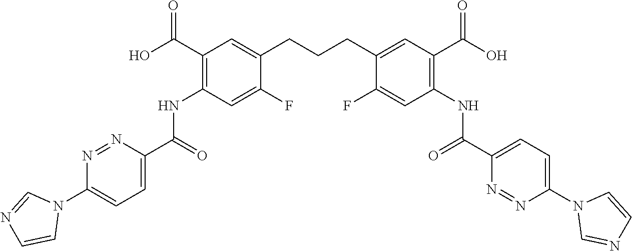 [SMILES: O=C(Nc1cc(F)c(CCCc2cc(C(=O)O)c(NC(=O)c3ccc(-n4ccnc4)nn3)cc2F)cc1C(=O)O)c1ccc(-n2ccnc2)nn1] | ¹H NMR (400 MHz, DMSO-d₆) δ 15.52 (s, 2H), 8.74 (d, J = 7.3 Hz, 1H), 8.80 (s, 2H), 8.55-8.41 (m, 6H), 8.21 (s, 2H), 8.09, 7.93 (m, 2H), 7.26 (s, 4H), 3.08 (s, 1H), 1.88 (s, 2H), 1.26- 1.19 (m, 4H). MS-ESI: m/z 695.1 observed [M + H]⁺ |
| 173 | 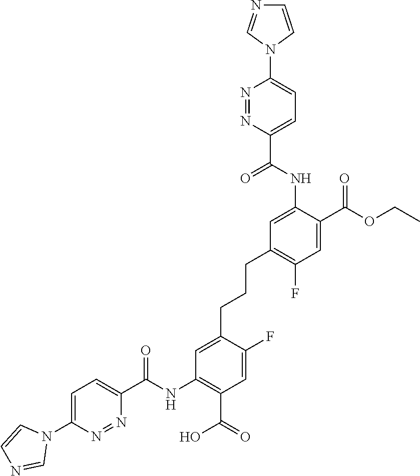 [SMILES: CCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] | MS-ESI: m/z 723.2 observed [M + H]⁺ |
| 174 | 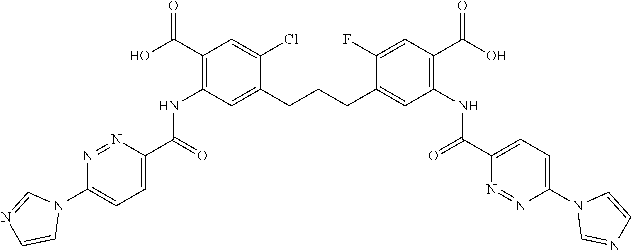 [SMILES: O=C(Nc1cc(CCCc2cc(NC(=O)c3ccc(-n4ccnc4)nn3)c(C(=O)O)cc2Cl)c(F)cc1C(=O)O)c1ccc(-n2ccnc2)nn1] | ¹H NMR (400 MHz, DMSO-d₆) δ = 15.86 (s, 1H), 15.81 (s, 1H), 8.78 (s, 3H), 8.74 (d, J = 7.6 Hz, 1H), 8.45 (d, J = 2.8 Hz, 1H), 8.42 (d, J = 3.2 Hz, 1H), 8.38 (d, J = 1.2 Hz, 1H), 8.36 (d, J = 0.8 Hz, 1H), 8.18 (s, 2H), 7.98 (s, 1H), 7.69 (d, J = 10.8 Hz, 1H), 7.24 (s, 2H), 2.81-2.73 (m, 4H), 1.96-1.94 (m, 2H) MS-ESI: m/z 711.1 observed [M + H]⁺ |
| 175 | 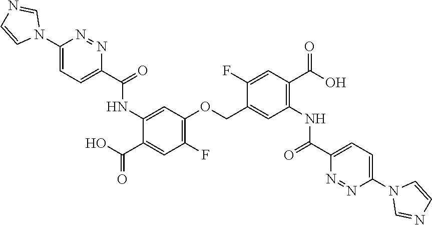 [SMILES: O=C(Nc1cc(COc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 15.42 (s, 1H), 15.21 (s, 1H), 8.91 (s, 1H), 8.82- 8.78 (m, 3H), 8.49- 8.41 (m, 4H), 8.19 (s, 2H), 7.86-7.83 (m, 2H), 7.25 (s, 2H), 5.33 (s, 2H). MS-ESI: m/z 683.1 observed [M + H]⁺ |
| 176 | 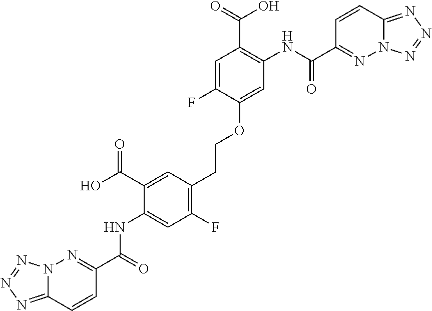 [SMILES: O=C(Nc1cc(OCCc2cc(C(=O)O)c(NC(=O)c3ccc4nnnn4n3)cc2F)c(F)cc1C(=O)O)c1ccc2nnnn2n1] | ¹H NMR (500 MHz, DMSO-d₆) δ 8.74 (d, J = 9.2 Hz, 1H), 8.48 (d, J = 9.2 Hz, 1H), 8.39-8.37 (m, 2H), 8.28 (d, J = 7.6 Hz, 1H), 4.34 (s, 2H), 73.17 (s, 2H). MS-ESI: m/z 647.2 observed [M + H]⁺ |
| 177 | 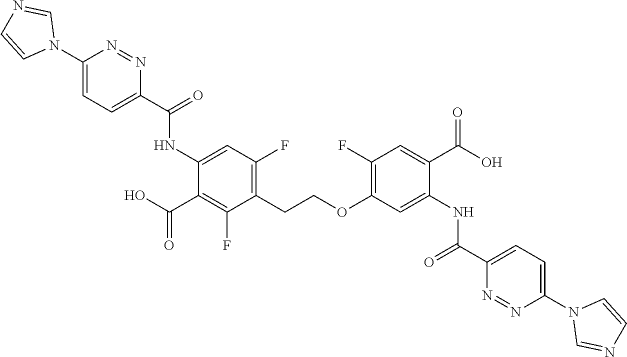 [SMILES: O=C(Nc1cc(OCCc2c(F)cc(NC(=O)c3ccc(-n4ccnc4)nn3)c(C(=O)O)c2F)c(F)cc1C(=O)O)c1ccc(-n2ccnc2)nn1] | ¹H NMR (400 MHz, DMSO-d₆) δ 14.48 (s, 1H), 8.78 (d, J = 8.9 Hz, 2H), 8.68 (d, J = 8.2 Hz, 1H), 8.50- 8.38 (m, 4H), 8.28 (d. J = 12.2 Hz, 1H), 8.20 (d, J = 7.7 Hz, 2H), 7.73 (d, J = 12.5 Hz, 1H), 7.25 (d, J = 4.6 Hz, 2H), 4.24 (t, J = 7.1 Hz, 2H), 3.14 (t, J = 7.1 Hz, 2H). MS-ESI: m/z 715.1 observed [M + H]⁺ |
| 178 | 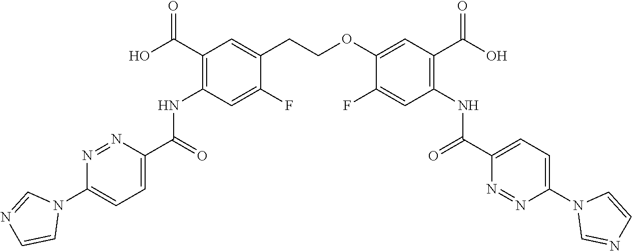 [SMILES: O=C(Nc1cc(F)c(CCOc2cc(C(=O)O)c(NC(=O)c3ccc(-n4ccnc4)nn3)cc2F)cc1C(=O)O)c1ccc(-n2ccnc2)nn1] | ¹H NMR (400 MHz, DMSO-d₆) δ 13.38 (s, 1H), 13.22 (s, 1H), 9.62 (s, 2H), 8.73- 8.50 (m, 8H), 8.24- 8.25 (m, 1H), 7.79 (m, 1H), 7.70 (s, 2H), 4.41-4.40 (m, 2H), 3.17-3.16 (m, 2H). MS-ESI: m/z 697.3 observed [M + H]⁺ |
| 179 | 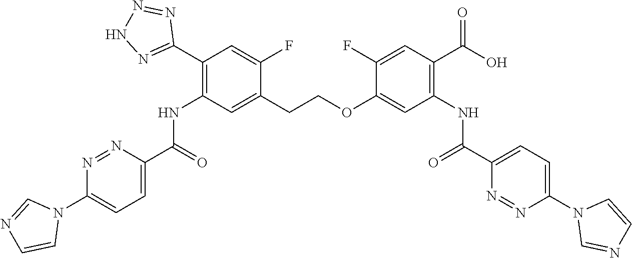 [SMILES: O=C(Nc1cc(OCCc2cc(NC(=O)c3ccc(-n4ccnc4)nn3)c(-c3nn[nH]n3)cc2F)c(F)cc1C(=O)O)c1ccc(-n2ccnc2)nn1] | ¹H NMR (400 MHz, DMSO-d₆) δ 9.61- 9.58 (m, 1H), 8.71- 8.69 (m, 2H), 8.46- 8.22 (m, 6H), 7.75- 7.64 (m, 5H), 4.48 (s, 2H), 3.48-3.34 (m, 2H. MS-ESI: m/z 721.2 observed [M + H]⁺ |
| 180 | 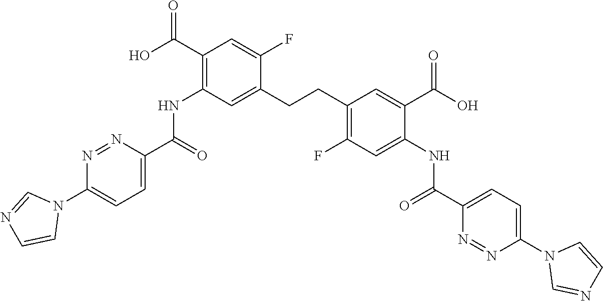 [SMILES: O=C(Nc1cc(CCc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 16.13 (s, 1H), 15.78 (s, 1H), 8.80-8.76 (m, 3H), 8.54-8.40 (m, 5H), 8.21 (s, 2H), 8.05 (d, J = 4.0 Hz, 2H), 7.72 (d, J = 12 Hz, 1H), 7.26 (s, 2H), 2.90 (s, 4H). MS-ESI: m/z 681.2 observed [M + H]⁺ |
| 181 | 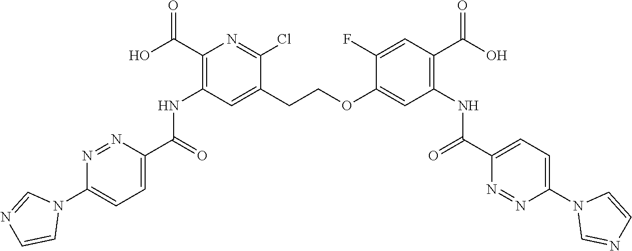 [SMILES: O=C(Nc1cc(OCCc2cc(NC(=O)c3ccc(-n4ccnc4)nn3)c(C(=O)O)nc2Cl)c(F)cc1C(=O)O)c1ccc(-n2ccnc2)nn1] | ¹H NMR (400 MHz, DMSO-d₆) δ 16.09 (s, 1H), 8.78 (s, 1H), 8.72 (d, J = 8 Hz, 3H), 8.48-8.39 (m, 3H), 8.34 (d, J = 8 Hz, 1H), 8.20 (s, 1H), 8.13 (s, 1H), 7.74 (d, J = 12 Hz, 1H), 7.25 (d, J = 8 Hz, 2H), 4.31 (s, 2H), 3.19 (s, 2H). MS-ESI: m/z 714.4 observed [M + H]⁺ |
| 182 | 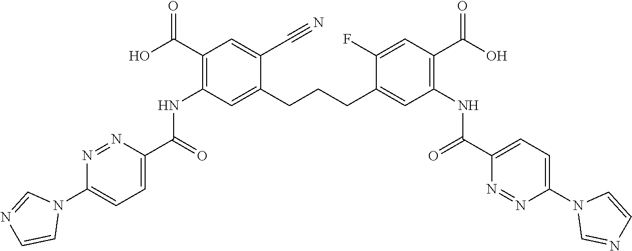 [SMILES: N#Cc1cc(C(=O)O)c(NC(=O)c2ccc(-n3ccnc3)nn2)cc1CCCc1cc(NC(=O)c2ccc(-n3ccnc3)nn2)c(C(=O)O)cc1F] | ¹H NMR (400 MHz, DMSO-d₆) δ 16.27 (s, 1H), 15.82 (s, 1H), 8.87-8.72 (m, 4H), 8.48-8.32 (m, 4H), 8.23-8.21 (m, 2H), 8.14-8.00 (m, 1H), 7.73-7.69 (m, 1H), 7.27-7.25 (m, 2H), 2.92 (s, 2H), 2.77-2.69 (m, 2H), 2.01-1.95 (m, 2H), 1.25 (s, 1H). MS-ESI: m/z 702.2 observed [M + H]⁺ |
| 183 | 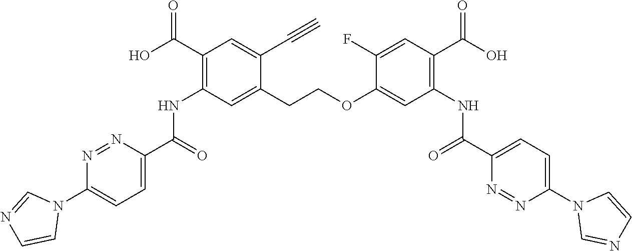 [SMILES: C#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] | ¹H NMR (400 MHz, DMSO-d₆) δ 16.08 (s, 2H), 8.85-8.70 (m, 4H), 8.47-8.45 (m, 2H), 8.42-8.39 (m, 2H), 8.21-8.18 (m, 3H), 7.77 (d, J = 12.4 Hz, 1H), 7.27 (s, 2H), 4.34-4.31 (m, 3H), 3.35-3.34 (m, 3H). MS-ESI: m/z 703.2 observed [M + H]⁺ |
| 184 | 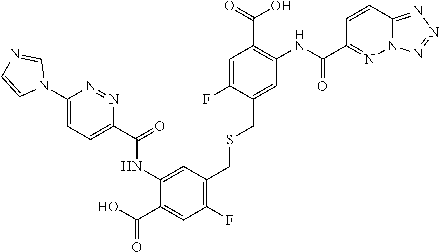 [SMILES: O=C(Nc1cc(CSCc2cc(NC(=O)c3ccc4nnnn4n3)c(C(=O)O)cc2F)c(F)cc1C(=O)O)c1ccc(-n2ccnc2)nn1] | ¹H NMR (400 MHz, DMSO-d₆) δ 16.34 (s, 1H), 15.72 (s, 1H), 8.93-8.78 (m, 4H), 8.44-8.33 (m, 3H), 8.19 (s, 1H), 3.84 (d, J = 4.4 Hz, 4H). MS-ESI: m/z 688.1 observed [M + H]⁺ |
| 185 | 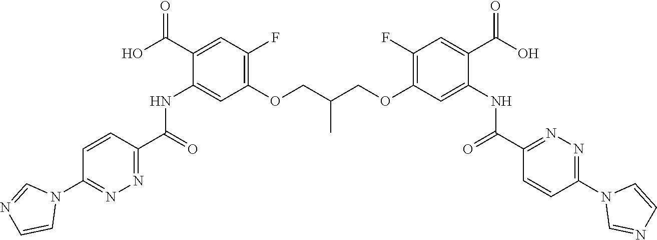 [SMILES: CC(COc1cc(NC(=O)c2ccc(-n3ccnc3)nn2)c(C(=O)O)cc1F)COc1cc(NC(=O)c2ccc(-n3ccnc3)nn2)c(C(=O)O)cc1F] | MS-ESI: m/z 741.41 observed [M + H]⁺ |
| 186 | 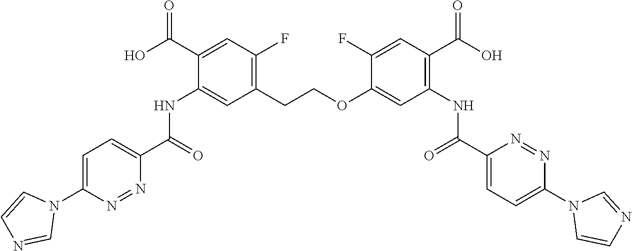 [SMILES: O=C(Nc1cc(CCOc2cc(NC(=O)c3ccc(-n4ccnc4)nn3)c(C(=O)O)cc2F)c(F)cc1C(=O)O)c1ccc(-n2ccnc2)nn1] | MS-ESI: m/z 696.2 observed [M + H]⁺ |
| 187 | 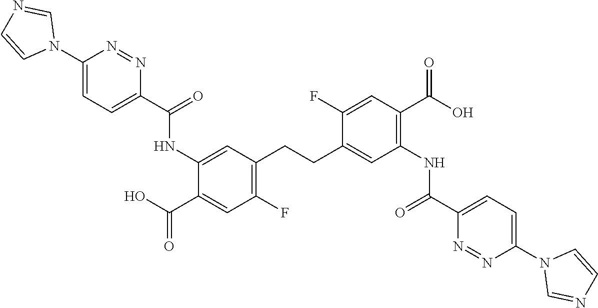 [SMILES: O=C(Nc1cc(CCc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 8.81- 8.73 (m, 4H), 8.47 (d, J = 9.1 Hz, 2H), 8.40 (d, J = 9.1 Hz, 2H), 8.19 (t, J = 1.4 Hz, 2H), 7.70 (d, J = 10.9 Hz, 2H), 7.25 (dd, J = 1.6, 0.8 Hz, 2H), 2.92 (s, 4H). MS-ESI: m/z 681.75 observed [M + H]⁺ |
| 188 | 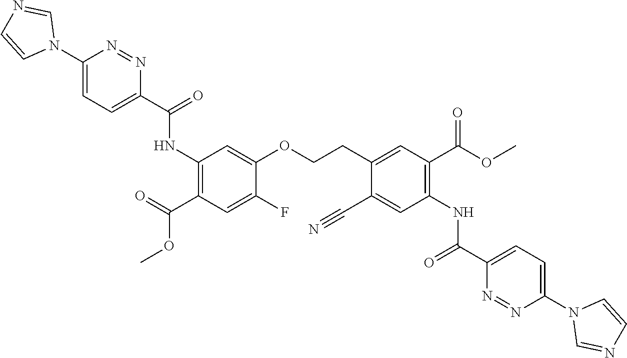 [SMILES: COC(=O)c1cc(F)c(OCCc2cc(C(=O)OC)c(NC(=O)c3ccc(-n4ccnc4)nn3)cc2C#N)cc1NC(=O)c1ccc(-n2ccnc2)nn1] | ¹H NMR (400 MHz, DMSO-d₆) δ 13.14 (s, 1H), 12.94 (s, 1H), 9.84 (s, 2H), 9.08 (s, 1H), 8.67-8.58 (m, 6H), 8.36 (s, 1H), 7.84- 7.37 (m, 3H), 4.54 (t, J = 6 Hz, 2H), 3.99 (s, 3H), 3.91 (s, 3H), 3.43 (t, J = 5.2 Hz, 2H). MS-ESI: m/z 732.1 observed [M + H]⁺ |
| 189 | 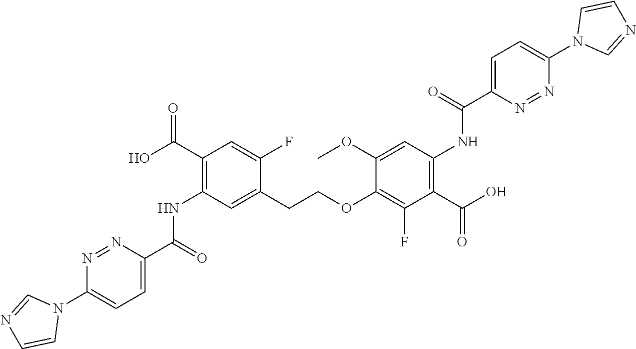 [SMILES: COc1cc(NC(=O)c2ccc(-n3ccnc3)nn2)c(C(=O)O)c(F)c1OCCc1cc(NC(=O)c2ccc(-n3ccnc3)nn2)c(C(=O)O)cc1F] | ¹H NMR (400 MHz, DMSO-d₆) δ 15.80 (s, 1H), 8.79-8.74 (m, 3H), 8.54-8.42 (m, 3H), 8.20 (s, 2H), 7.90- 7.65 (m, 2H), 7.26 (s, 2H), 4.26 (s, 2H), 3.87 (s, 3H), 3.06 (d, J = 7.2 Hz, 2H), 2.08 (s, 4H). MS-ESI: m/z 727.2 observed [M + H]⁺ |
| 190 | 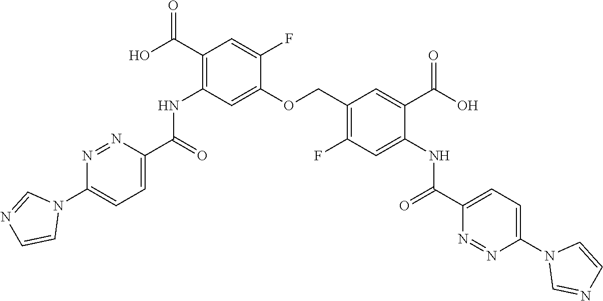 [SMILES: O=C(Nc1cc(OCc2cc(C(=O)O)c(NC(=O)c3ccc(-n4ccnc4)nn3)cc2F)c(F)cc1C(=O)O)c1ccc(-n2ccnc2)nn1] | ¹H NMR (400 MHz, DMSO-d₆) δ 16.31 (s, 1H), 16.05 (s, 1H), 8.79 (d, J = 4.4 Hz, 3H), 8.63 (s, 1H), 8.50-8.40 (m, 4H), 8.21 (m, 1H), 8.21-8.20 (m, 3H), 7.78-7.76 (m, 1H), 7.26 (s,2H), 5.20 (s, 2H). MS-ESI: m/z 683.2 observed [M + H]⁺ |
| 191 | 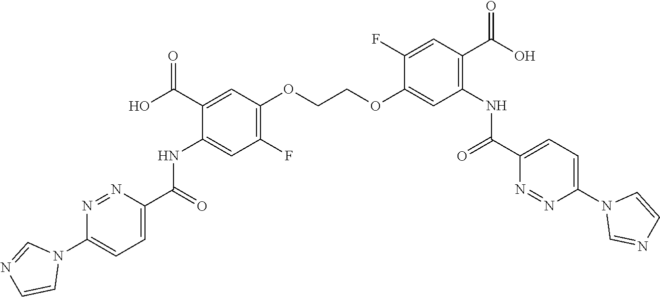 [SMILES: O=C(Nc1cc(OCCOc2cc(C(=O)O)c(NC(=O)c3ccc(-n4ccnc4)nn3)cc2F)c(F)cc1C(=O)O)c1ccc(-n2ccnc2)nn1] | MS-ESI: m/z 713.2 observed [M + H]⁺ |
| 192 | 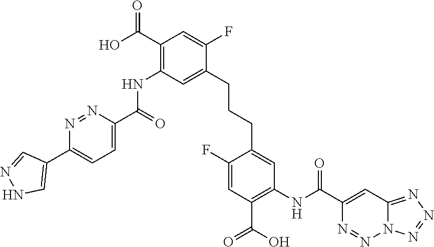 [SMILES: O=C(Nc1cc(CCCc2cc(NC(=O)c3cc4nnnn4nn3)c(C(=O)O)cc2F)c(F)cc1C(=O)O)c1ccc(-c2cn[nH]c2)nn1] | MS-ESI: m/z 671.31 observed [M + H]⁺ |
| 193 | 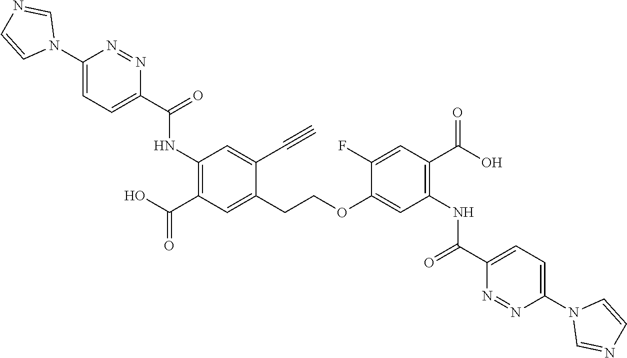 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1F] | ¹H NMR (400 MHz, DMSO-d₆) δ 16.05 (s, 1H), 15.95 (s, 1H), 8.90 (s, 1H), 8.79 (d, J = 9.6 Hz, 2H), 8.70 (d, J = 8 Hz, 1H), 8.45 (dd, J = 25.6, 8.8 Hz, 4H), 8.21 (d, J = 9.2 Hz, 2H), 8.09 (s, 1H), 7.75 (d, J = 12.4 Hz, 1H), 7.26 (d, J = 5.6 Hz, 2H), 4.45 (s, 1H), 4.27 (s, 2H), 3.35- 3.28 (m, 2H). MS-ESI: m/z 703.2 observed [M + H]⁺ |
| 194 | 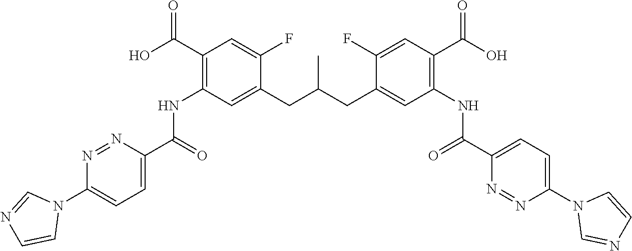 [SMILES: CC(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] | ¹H NMR (400 MHz, DMSO-d₆) δ 14.52 (s, 2H), 8.79-8.34 (m, 3H), 8.44 (dd, J = 26.8, 8.8 Hz, 3H), 8.19 (s, 2H), 7.71 (d, J = 10.4, 2H), 7.26 (s, 4H), 3.14- 3.11 (m, 1H), 2.81- 2.60 (m, 4H), 0913 (d, J = 6 Hz, 3H). MS-ESI: m/z 709.2 observed [M + H]⁺ |
| 195 | 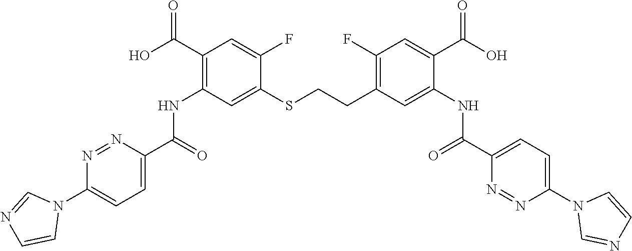 [SMILES: O=C(Nc1cc(CCSc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 15.84 (d, J = 35.2 Hz, 2H), 8.97 (d, J = 7.6 Hz, 1H), 8.77 (d, J = 6.4 Hz, 3H), 8.46 (d, J = 9.2 Hz, 1H), 8.35- 8.33 (m, 3H), 8.18 (s, 2H), 7.74 (dd, J = 17.2, 10.8 Hz, 2H), 7.25 (d, J = 8.8 Hz, 2H), 3.36-3.31 (m, 2H), 3.11-3.09 (m, 2H). MS-ESI: m/z 713.2 observed [M + H]⁺ |
| 196 | 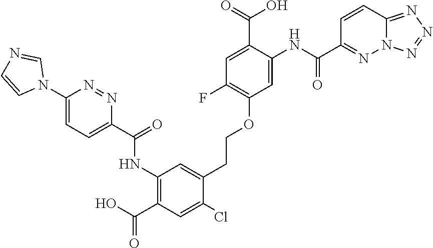 [SMILES: O=C(Nc1cc(CCOc2cc(NC(=O)c3ccc4nnnn4n3)c(C(=O)O)cc2F)c(Cl)cc1C(=O)O)c1ccc(-n2ccnc2)nn1] | ¹H NMR (400 MHz, DMSO-d₆) δ 8.93 (s, 1H), 8.90 (d, J = 6.4 Hz, 1H), 8.78 (s, 1H), 8.42 (d, J = 17.2 Hz, 1H), 8.44-8.32 (m, 3H), 8.19 (s, 1H), 8.05 (s, 1H), 7.75 (d, J = 12.4 Hz, 1H), 7.25 (s, 2H), 4.34 (t, J = 6.8 Hz, 2H), 3.32- 3.25 (m, 2H). MS-ESI: m/z 688.3 observed [M + H]⁺ |
| 197 | 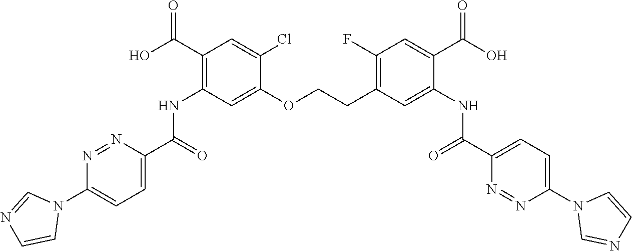 [SMILES: O=C(Nc1cc(CCOc2cc(NC(=O)c3ccc(-n4ccnc4)nn3)c(C(=O)O)cc2Cl)c(F)cc1C(=O)O)c1ccc(-n2ccnc2)nn1] | ¹H NMR (400 MHz, DMSO-d₆) δ 14.44 (s, 1H), 8.92 (d, J = 6.8, 1H), 8.77 (d, J = 22 Hz, 3H), 8.52- 8.41 (m, 4H), 8.20 (s, 2H), 8.01 (s, 1H), 7.77 (d, J = 10.4 Hz, 1H), 7.26 (s, 2H), 4.42 (s, 2H), 3.35-3.27 (m, 2H), 3.22 (s, 2H), 0.97 (s, 2H). MS-ESI: m/z 714.3 observed [M + H]⁺ |
| 198 | 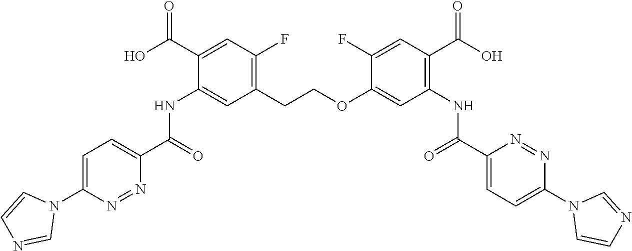 [SMILES: O=C(Nc1cc(CCOc2cc(NC(=O)c3ccc(-n4ccnc4)nn3)c(C(=O)O)cc2F)c(F)cc1C(=O)O)c1ccc(-n2ccnc2)nn1] | 1H NMR (400 MHz, DMSO-d₆) δ 15.74 (s, 1H), 11.20 (s, 1H), 8.81-8.71 (m, 3H), 8.51-8.43 (m, 4H), 8.20-8.12 (m, 3H), 7.81 (d, J = 12 Hz, 1H), 7.28 (s, 3H), 5.86 (s. 1H), 4.64 (s, 2H), 4.34 (s, 2H), 3.22 (s, 2H), 0.97 (s, 2H). MS-ESI: m/z 683.3 observed [M + H]⁺ |
| 199 | 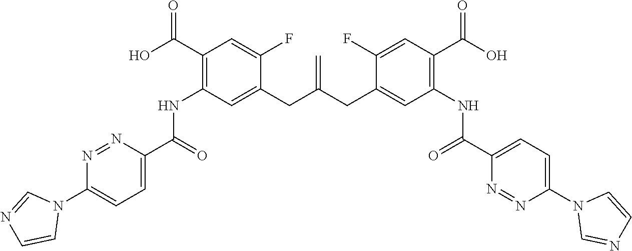 [SMILES: C=C(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] | 1H NMR (400 MHz, DMSO-d₆) δ 14.10 (s, 2H), 8.75-8.73 (m, 4H), 8.38 (dd, J = 20.4, 9.2 Hz, 4H), 8.15 (s, 2H), 7.71 (d, J = 10.4 Hz, 2H), 7.23 (s, 2H), 4.94 (s, 2H), 3.47 (s, 4H). MS-ESI: m/z 707.0 observed [M + H]⁺ |
| 200 | 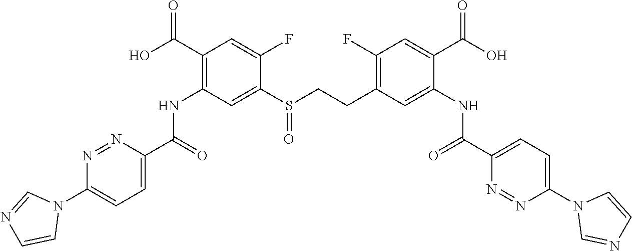 [SMILES: O=C(Nc1cc(CCS(=O)c2cc(NC(=O)c3ccc(-n4ccnc4)nn3)c(C(=O)O)cc2F)c(F)cc1C(=O)O)c1ccc(-n2ccnc2)nn1] | 1H NMR (400 MHz, DMSO-d₆) δ 8.81- 8.71 (m, 2H), 8.53-8.46 (m, 3H), 8.38- 8.33 (m, 2H), 8.22-8.15 (m, 2H), 8.01 (d, J = 8 Hz, 1H), 7.53- 7.49 (m, 1H), 7.40 (s, 1H), 7.26 (s, 2H), 3.26 (m, 2H), 3.04-3.02 (m, 3H) 2.74-2.70 (m, 1H). MS-ESI: m/z 729.1 observed [M + H]⁺ |
| 201 | 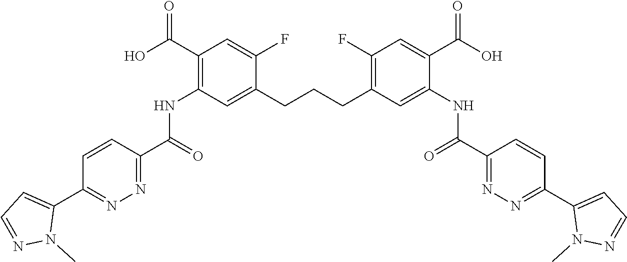 [SMILES: Cn1nccc1-c1ccc(C(=O)Nc2cc(CCCc3cc(NC(=O)c4ccc(-c5ccnn5C)nn4)c(C(=O)O)cc3F)c(F)cc2C(=O)O)nn1] | 1H NMR (500 MHz, DMSO-d₆) δ 13.09 (s, 2H), 8.82 (d, J = 7.0 Hz, 2H), 8.34- 8.28 (m, 4H), 7.73 (d, J = 10.3 Hz, 2H), 7.61 (d, J = 2.0 Hz, 2H), 7.04 (d, J = 2.0 Hz, 2H), 4.25 (s, 6H), 2.84 (t. J = 7.5 Hz, 4H), 1.23 (s, 1H). MS-ESI: m/z 723.5 observed [M + H]⁺ |
| 202 | 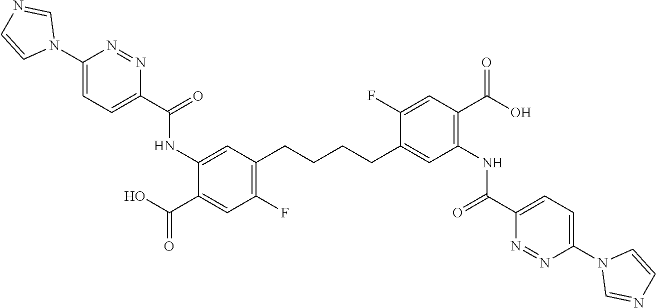 [SMILES: O=C(Nc1cc(CCCCc2cc(NC(=O)c3ccc(-n4ccnc4)nn3)c(C(=O)O)cc2F)c(F)cc1C(=O)O)c1ccc(-n2ccnc2)nn1] | 1H NMR (500 MHz, DMSO-d₆) δ 8.76 (t, J = 1.1 Hz, 2H), 8.70 (d, J = 7.3 Hz, 3H), 8.45-8.34 (m, SH), 8.17 (t, J = 1.4 Hz, 3H), 7.67 (d, J = 11.0 Hz, 3H), 7.24 (dd, J = 1.5, 0.8 Hz, 3H), 1.68 (s, 6H), 1.23 (s, 4H). MS-ESI: m/z 709.46 observed [M + H]⁺ |
| 203 | 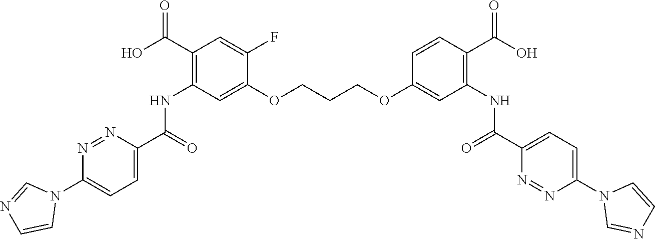 [SMILES: O=C(Nc1cc(OCCCOc2cc(NC(=O)c3ccc(-n4ccnc4)nn3)c(C(=O)O)cc2F)ccc1C(=O)O)c1ccc(-n2ccnc2)nn1] | 1H NMR (500 MHz, DMSO-d₆) δ 8.79- 8.76 (m, 1H), 8.72 (d, J = 8.2 Hz, 1H), 8.48-8.44 (m, 2H), 8.39 (dd, J = 9.1, 2.1 Hz, 1H), 8.18 (q, J = 1.6 Hz, 1H), 8.00 (d, J = 8.6 Hz, 1H), 7.75 (d, J = 12.6 Hz, 1H), 7.25 (d, J = 2.1 Hz, 1H), 4.26 (dt, J = 31.1, 6.3 Hz, 3H), 1.23 (s, 2H). MS-ESI: m/z 709.31 observed [M + H]⁺ |
| 204 | 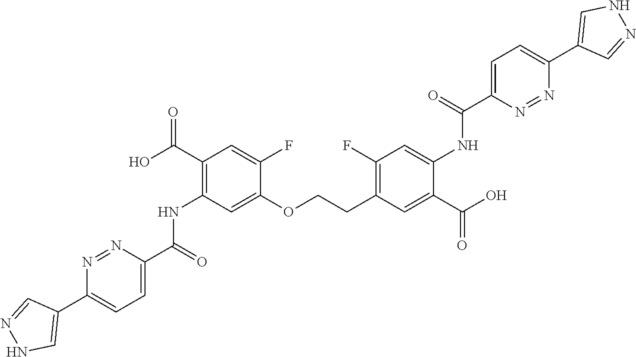 [SMILES: O=C(Nc1cc(OCCc2cc(C(=O)O)c(NC(=O)c3ccc(-c4cn[nH]c4)nn3)cc2F)c(F)cc1C(=O)O)c1ccc(-c2cn[nH]c2)nn1] | 1H NMR (400 MHz, DMSO-d₆) δ 15.93 (s, 1H), 15.77 (s, 1H), 13.53 (s, 1H), 8.74 (d, J = 8 Hz, 1H), 8.59 (d, J = 12.8 Hz, 1H), 8.51 (s, 1H), 8.27- 8.14 (m, 5H), 7.78 (d, J = 12.8, 1H), 4.30 (s, 1H), 3.17 (s, 1H), 1.62 (s, 1H). MS-ESI: m/z 697.2 observed [M + H]⁺ |
| 205 | 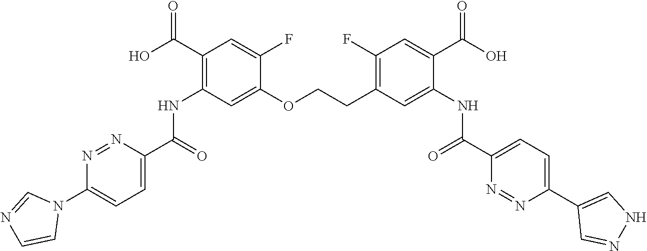 [SMILES: O=C(Nc1cc(CCOc2cc(NC(=O)c3ccc(-n4ccnc4)nn3)c(C(=O)O)cc2F)c(F)cc1C(=O)O)c1ccc(-c2cn[nH]c2)nn1] | ¹H NMR (400 MHz, DMSO-d₆) δ 15.96 (s, 1H), 8.86 (d, J = 7.2 Hz, 1H), 8.79 (s, 1H), 8.73 (d, J = 8.4 Hz, 1H), 8.48-8.40 (m, 4H), 8.23-8.15 (m, 3H), 7.77 (d, J = 11.6 Hz, 2H), 7.27 (s, 1H), 4.35 (s, 2H), 3.20 (s, 2H). MS-ESI: m/z 697.2 observed [M + H]⁺ |
| 206 | 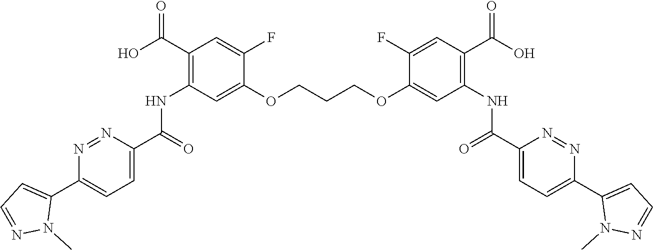 [SMILES: Cn1nccc1-c1ccc(C(=O)Nc2cc(OCCCOc3cc(NC(=O)c4ccc(-c5ccnn5C)nn4)c(C(=O)O)cc3F)c(F)cc2C(=O)O)nn1] | ¹H NMR (400 MHz, DMSO-d₆) δ 16.04 (s, 2H), 8.74 (d, J = 8.1 Hz, 2H), 8.40- 8.28 (m, 3H), 7.74 (d, J = 12.6 Hz, 1H), 7.62 (d. J = 2.0 Hz, 1H), 7.08 (d, J = 2.0 Hz, 2H), 4.29 (s, 6H), 1.23 (s, 5H). MS-ESI: m/z 755.54 observed [M + H]⁺ |
| 207 | 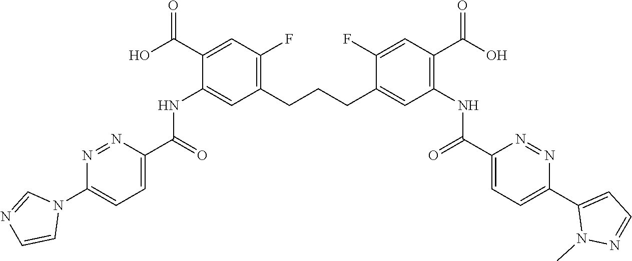 [SMILES: Cn1nccc1-c1ccc(C(=O)Nc2cc(CCCc3cc(NC(=O)c4ccc(-n5ccnc5)nn4)c(C(=O)O)cc3F)c(F)cc2C(=O)O)nn1] | ¹H NMR (400 MHz, DMSO-d₆) δ 15.51 (br d, J = 8.0 Hz, 2H), 8.86-8.65 (m, 3H), 8.44-8.37 (m, 2H), 8.35-8.25 (m, 2H), 8.18 (s, 1H), 7.74 (br d, J = 10.8 Hz, 2H), 7.61 (s, 1H), 7.25 (s, 1H), 7.06 (s, 1H), 4.28 (s, 3H), 2.73 (br d, J = 6.8 Hz, 4H), 1.94 (br d, J = 6.4 Hz, 2H). LCMS [ESI, M + 1]: 709.1 |
| 208 | 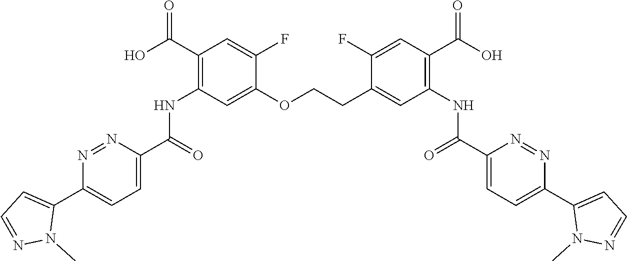 [SMILES: Cn1nccc1-c1ccc(C(=O)Nc2cc(CCOc3cc(NC(=O)c4ccc(-c5ccnn5C)nn4)c(C(=O)O)cc3F)c(F)cc2C(=O)O)nn1] | ¹H NMR (400 MHz, DMSO-d₆) δ 16.02 (s, 1H), 15.78 (s, 1H), 8.85 (d, J = 7.2 Hz, 1H), 8.72 (d, J = 8.2 Hz, 1H), 8.37-8.27 (m, 4H), 7.76-7.71 (m, 2H), 7.61 (t, J = 1.8 Hz, 2H), 7.08 (dd, J = 2.0, 1.1 Hz, 2H), 4.32 (t, J = 7.0 Hz, 2H), 4.29 (d, J = 2.0 Hz, 6H), 3.19 (t, J = 6.8 Hz, 2H). MS-ESI: m/z 725.18 observed [M + H]⁺ |
| 209 | 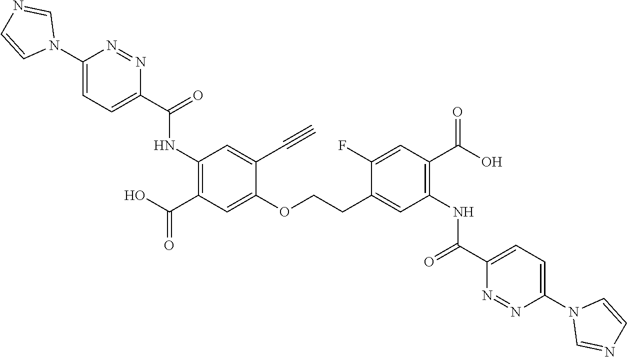 [SMILES: C#Cc1cc(NC(=O)c2ccc(-n3ccnc3)nn2)c(C(=O)O)cc1OCCc1cc(NC(=O)c2ccc(-n3ccnc3)nn2)c(C(=O)O)cc1F] | MS-ESI: m/z 703.2 observed [M + H]⁺ |
| 210 | 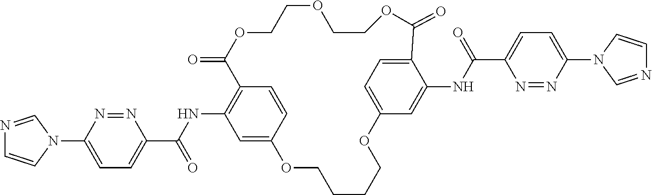 [SMILES: O=C(Nc1cc2ccc1C(=O)OCCOCCOC(=O)c1ccc(cc1NC(=O)c1ccc(-n3ccnc3)nn1)OCCCCO2)c1ccc(-n2ccnc2)nn1] | ¹H NMR (400 MHz, DMSO-d₆) δ 13.01 (s, 2H), 10.19-10.18 (m, 2H), 8.65-8.64 (m, 2H), 8.56 (s, 4H), 8.36 (d, J = 4 Hz, 2H), 7.97-7.96 (m, 2H), 7.63 (d, J = 4 Hz, 2H), 6.31-6.29 (m, 2H), 4.44-4.42 (m, 4H), 4.29 (t, J = 4 Hz, 4H) 3.83-3.81 (m, 4H), 2.03-2.02 (m, 4H). MS-ESI: m/z 775.25 observed [M + H]⁺ |
| 211 | 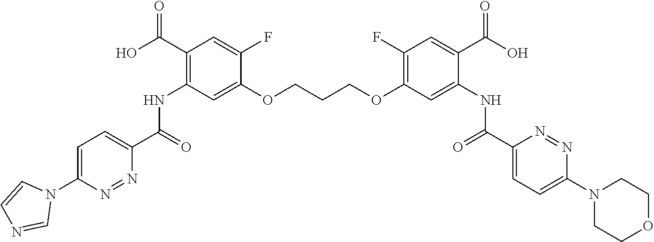 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCOCC2)nn1] | ¹H NMR (400 MHz, DMSO-d₆) δ 15.03- 15.00 (m, 1H), 14.00- 13.94 (m, 1H), 8.80-8.74 (m, 2H), 8.44 (dd, J = 24.8, 9.2 Hz, 2H), 8.21 (s, 1H), 7.97 (d, J = 9.6 Hz, 1H), 7.77 (dd, J = 12, 7.2 Hz, 2H), 7.40 (d, J = 2.4 Hz, 1H), 7.27 (s, 3H), 4.34 (t, J = 5.6 Hz, 4H) 3.73 (d, J = 5.6 Hz, 8H), 2.39-2.34 (m, 2H). MS-ESI: m/z 746.2 observed [M + H]⁺ |
| 212 | 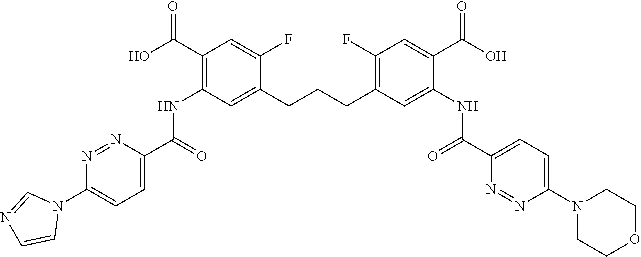 [SMILES: O=C(Nc1cc(CCCc2cc(NC(=O)c3ccc(-n4ccnc4)nn3)c(C(=O)O)cc2F)c(F)cc1C(=O)O)c1ccc(N2CCOCC2)nn1] | ¹H NMR (400 MHz, DMSO-d₆) δ 15.72 (s, 1H), 15.10 (s, 1H), 8.81-8.79 (m, 3H), 8.42 (dd, J = 23.2, 9.2 Hz, 2H), 8.21 (s, 1H), 7.94 (d, J = 9.6 Hz, 1H), 7.70 (t, J = 10.8 Hz, 2H), 7.39- 7.35 (m, 1H), 7.28- 7.25 (m, 1H), 3.74 (d, J = 20 Hz, 8H) 2.72-2.63 (m, 4H), 1.95 (s, 2H). MS-ESI: m/z 714.4 observed [M + H]⁺ |
| 213 | 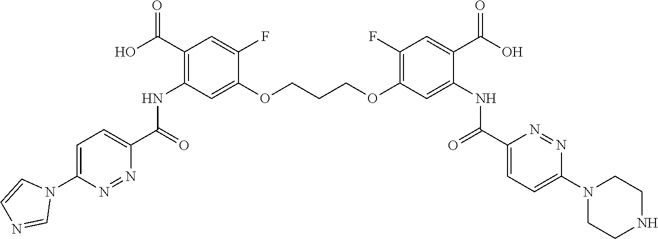 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCNCC2)nn1] | ¹H NMR (400 MHz, DMSO-d₆) δ 16.03 (s, 1H), 15.32 (s, 1H), 8.80 (s, 1H), 8.74- 8.71 (m, 2H), 8.51- 8.8.49 (m, 1H), 8.43- 8.33 (m, 1H), 8.21-8.13 (m, 2H), 7.95- 7.91 (m, 1H), 7.95- 7.91 (m, 2H), 7.55 (d, J = 13.6 Hz, 1H) 7.35- 7.25 (m, 2H), 4.30- 4.20 (m, 4H), 3.67- 3.57 (m, 4H), 2.83 (s, 4H), 2.35 (s, 2H). MS-ESI: m/z 745.2 observed [M + H]⁺ |
| 214 | 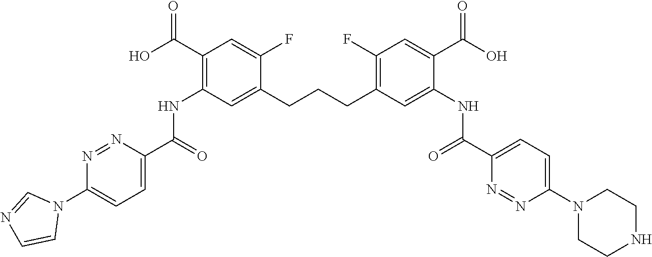 [SMILES: O=C(Nc1cc(CCCc2cc(NC(=O)c3ccc(-n4ccnc4)nn3)c(C(=O)O)cc2F)c(F)cc1C(=O)O)c1ccc(N2CCNCC2)nn1] | ¹H NMR (400 MHz, DMSO-d₆) δ 14.87 (s, 1H), 14.25 (s, 1H), 8.76-8.71 (m, 3H), 8.43-8.37 (m, 2H), 8.16 (s, 1H), 7.88- 7.86 (m, 1H), 7.66 (t, J = 10.4 Hz, 2H), 7.24 (s, 1H), 4.05 (s, 4H), 2.76-2.71 (m, 4H), 1.99-1.92 (m, 2H). MS-ESI: m/z 713.4 observed [M + H]⁺ |
| 215 | 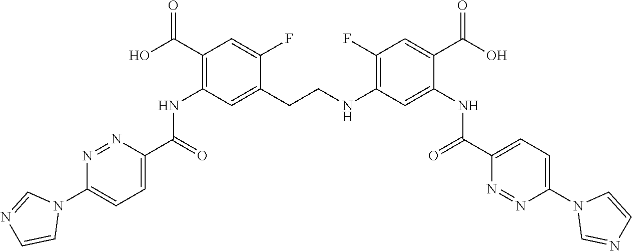 [SMILES: O=C(Nc1cc(CCNc2cc(NC(=O)c3ccc(-n4ccnc4)nn3)c(C(=O)O)cc2F)c(F)cc1C(=O)O)c1ccc(-n2ccnc2)nn1] | ¹H NMR (400 MHz, DMSO-d₆) δ 8.81- 8.77 (m, 3H), 8.52 (d, J = 9.2 Hz, 1H), 8.43-8.37 (m, 4H), 8.18 (s, 2H), 7.73 (d, J = 10.8 Hz, 1H), 7.61 (d, J = 12.4 Hz, 1H), 7.25 (d, J = 3.6 Hz, 1H), 3.51-3.48 (m, 2H), 3.04 (t, J = 7.2 Hz, 2H), MS-ESI: m/z 696.55 observed [M + H]⁺ |
| 216 | 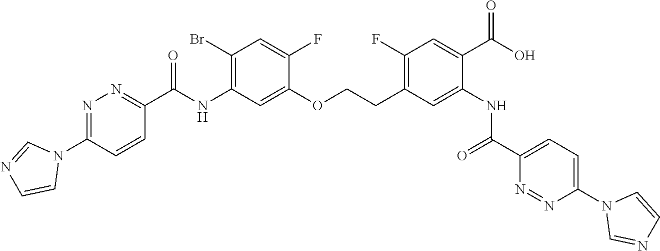 [SMILES: O=C(Nc1cc(OCCc2cc(NC(=O)c3ccc(-n4ccnc4)nn3)c(C(=O)O)cc2F)c(F)cc1Br)c1ccc(-n2ccnc2)nn1] | ¹H NMR (400 MHz, DMSO-d₆) δ 10.65 (s, 1H), 8.99-8.81 (m, 3H), 8.54-8.40 (m, 4H), 8.21 (s, 2H), 8.06 (d, J = 8.4 Hz, 1H), 7.76 (d, J = 10.4 Hz, 2H), 6.66 (s, 2H), 4.38 (t, J = 6.4 Hz, 2H), 3.21 (s, 2H. MS-ESI: m/z 731.2 observed [M + H]⁺ |
| 217 | 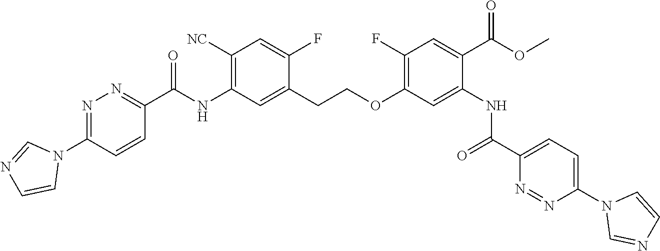 [SMILES: COC(=O)c1cc(F)c(OCCc2cc(NC(=O)c3ccc(-n4ccnc4)nn3)c(C#N)cc2F)cc1NC(=O)c1ccc(-n2ccnc2)nn1] | ¹H NMR (400 MHz, DMSO-d₆) δ 13.12 (s, 1H), 11.38 (s, 1H), 8.82-8.75 (m, 3H), 8.49 (dd, J = 8.8, 17.4 Hz, 1H), 8.21 (d, J = 8.8 Hz, 2H), 7.99-7.93 (m, 2H), 7.83 (d, J = 11.6 Hz, 1H), 7.27 (s, 2H), 4.49 (s, 2H), 3.91 (s, 3H), MS-ESI: m/z 692.1 observed [M + H]⁺ |
| 218 | 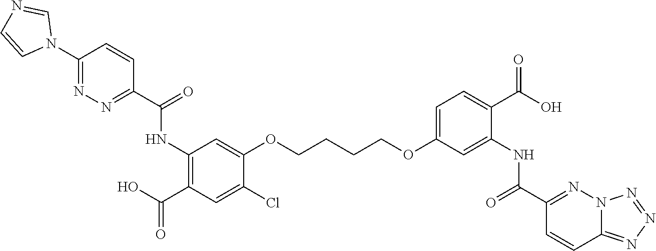 [SMILES: O=C(Nc1cc(OCCCCOc2ccc(C(=O)O)c(NC(=O)c3ccc4nnnn4n3)c2)c(Cl)cc1C(=O)O)c1ccc(-n2ccnc2)nn1] | MS-ESI: m/z 715.50 observed [M + H]⁺ |
| 219 | 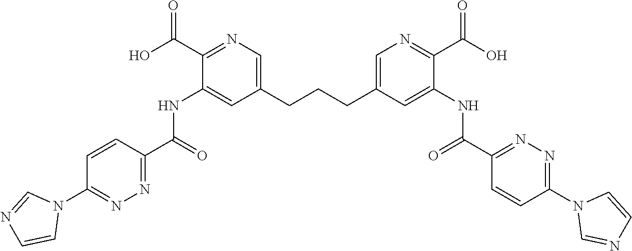 [SMILES: O=C(Nc1cc(CCCc2cnc(C(=O)O)c(NC(=O)c3ccc(-n4ccnc4)nn3)c2)cnc1C(=O)O)c1ccc(-n2ccnc2)nn1] | ¹H NMR (400 MHz, DMSO-d₆) δ 10.00 (s, 2H), 9.56 (s, 2H), 8.61 (d, J = 9.2 Hz, 2H), 8.50 (s, 2H), 8.27 (d, J = 9.2 Hz, 2H), 8.18 (s, 2H), 7.57 (s, 2H), 3.83 (s, 3H), 3.12 (s, 4H), 2.22-2.14 (m, 2H). MS-ESI: m/z 661.3 observed [M + H]⁺ |
| 220 | 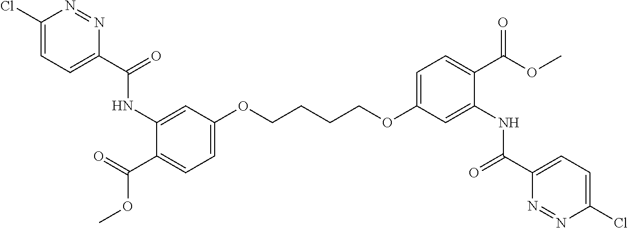 [SMILES: COC(=O)c1ccc(OCCCCOc2ccc(C(=O)OC)c(NC(=O)c3ccc(Cl)nn3)c2)cc1NC(=O)c1ccc(Cl)nn1] | ¹H NMR (400 MHz, DMSO-d₆) δ 12.84 (s, 2H), 8.40 (d, J = 2.8 Hz, 2H), 8.33 (d, J = 8.8 Hz , 2H), 8.12 (d. J = 8.8 Hz, 2H), 8.01 (d, J = 9.2 Hz, 2H), 8.12 (dd, J = 8.8, 3.2 Hz, 2H), 4.25-4.23 (m, 4H), 2.01-1.98 (m, 4H). MS-ESI: m/z 669.2 observed [M+H]+ |
| 221 | 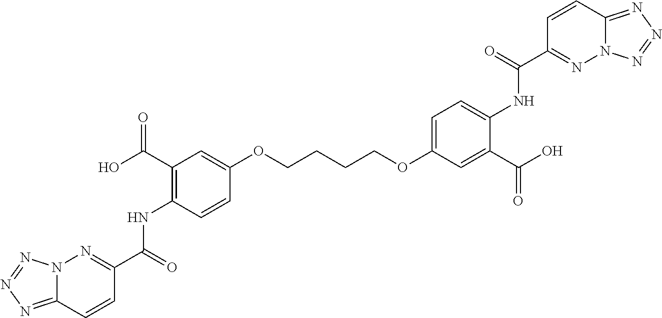 [SMILES: O=C(Nc1ccc(OCCCCOc2ccc(NC(=O)c3ccc4nnnn4n3)c(C(=O)O)c2)cc1C(=O)O)c1ccc2nnnn2n1] | MS-ESI: m/z 655.2 observed [M + H]⁺ |
| 222 | 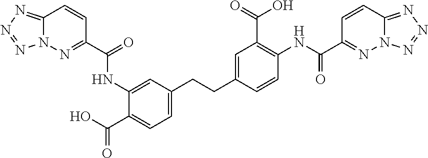 [SMILES: O=C(Nc1cc(CCc2ccc(NC(=O)c3ccc4nnnn4n3)c(C(=O)O)c2)ccc1C(=O)O)c1ccc2nnnn2n1] | MS-ESI: m/z 595.2 observed [M + H]⁺ |
| 223 | 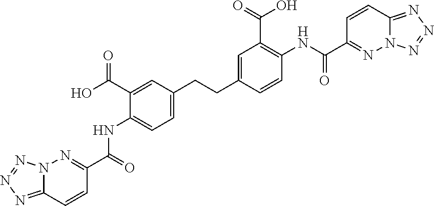 [SMILES: O=C(Nc1ccc(CCc2ccc(NC(=O)c3ccc4nnnn4n3)c(C(=O)O)c2)cc1C(=O)O)c1ccc2nnnn2n1] | MS-ESI: m/z 595.2 observed [M + H]⁺ |
| 224 | 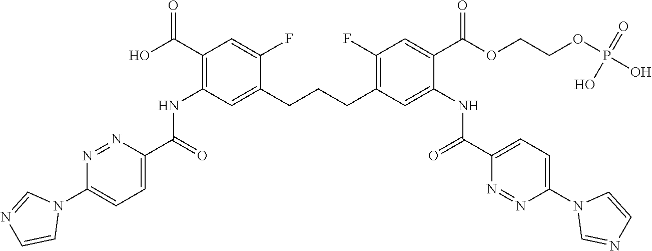 [SMILES: O=C(Nc1cc(CCCc2cc(NC(=O)c3ccc(-n4ccnc4)nn3)c(C(=O)OCCOP(=O)(O)O)cc2F)c(F)cc1C(=O)O)c1ccc(-n2ccnc2)nn1] | |
| 225 | 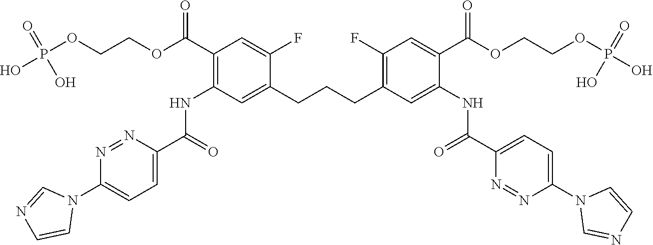 [SMILES: O=C(Nc1cc(CCCc2cc(NC(=O)c3ccc(-n4ccnc4)nn3)c(C(=O)OCCOP(=O)(O)O)cc2F)c(F)cc1C(=O)OCCOP(=O)(O)O)c1ccc(-n2ccnc2)nn1] | |
| 226 | 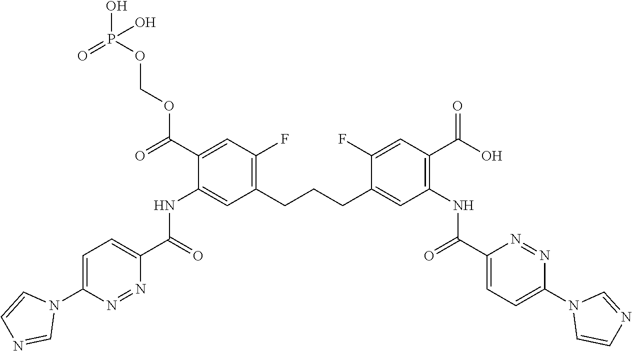 [SMILES: O=C(Nc1cc(CCCc2cc(NC(=O)c3ccc(-n4ccnc4)nn3)c(C(=O)OCOP(=O)(O)O)cc2F)c(F)cc1C(=O)O)c1ccc(-n2ccnc2)nn1] | |
| 227 | 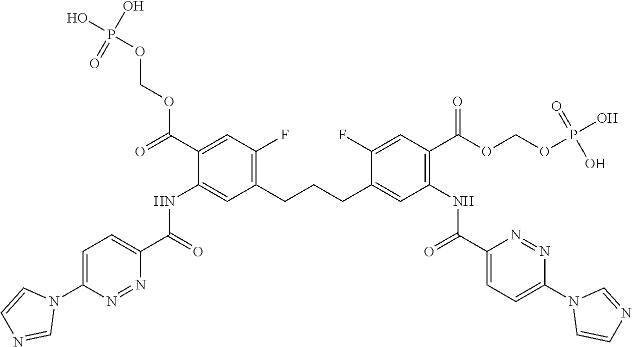 [SMILES: O=C(Nc1cc(CCCc2cc(NC(=O)c3ccc(-n4ccnc4)nn3)c(C(=O)OCOP(=O)(O)O)cc2F)c(F)cc1C(=O)OCOP(=O)(O)O)c1ccc(-n2ccnc2)nn1] | |
| 228 | 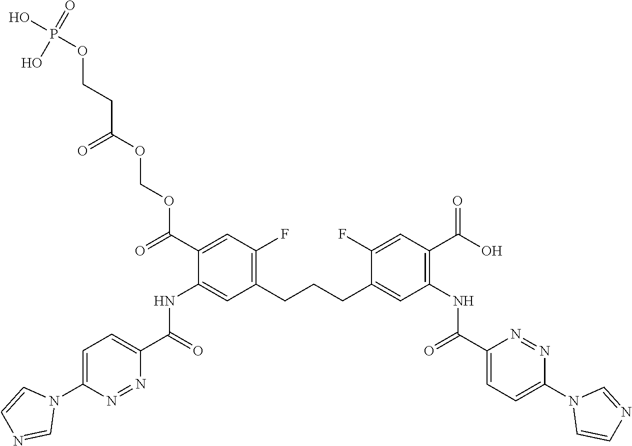 [SMILES: O=C(CCOP(=O)(O)O)OCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] | |
| 229 | 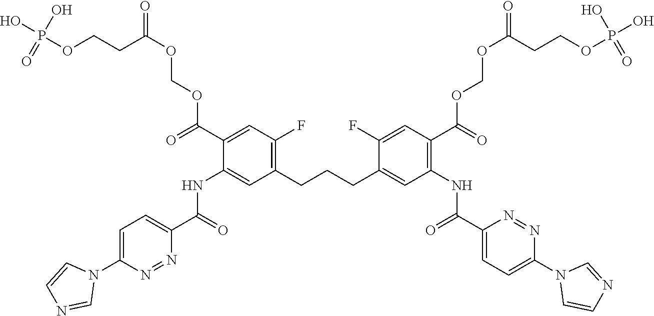 [SMILES: O=C(CCOP(=O)(O)O)OCOC(=O)c1cc(F)c(CCCc2cc(NC(=O)c3ccc(-n4ccnc4)nn3)c(C(=O)OCOC(=O)CCOP(=O)(O)O)cc2F)cc1NC(=O)c1ccc(-n2ccnc2)nn1] | |
| 230 | 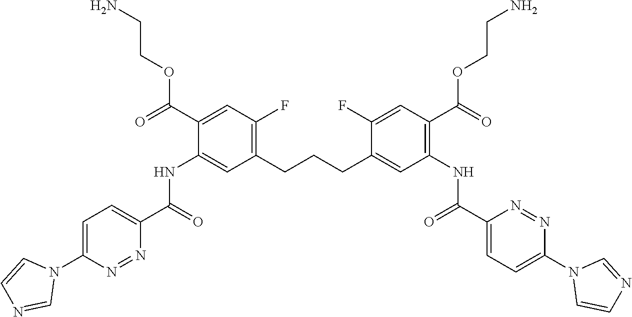 [SMILES: NCCOC(=O)c1cc(F)c(CCCc2cc(NC(=O)c3ccc(-n4ccnc4)nn3)c(C(=O)OCCN)cc2F)cc1NC(=O)c1ccc(-n2ccnc2)nn1] | |
| 231 | 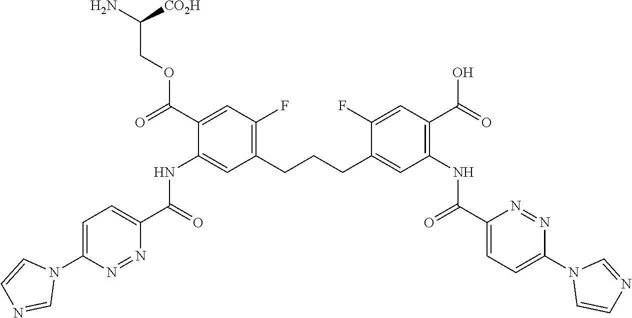 [SMILES: N[C@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] | |
| 232 | 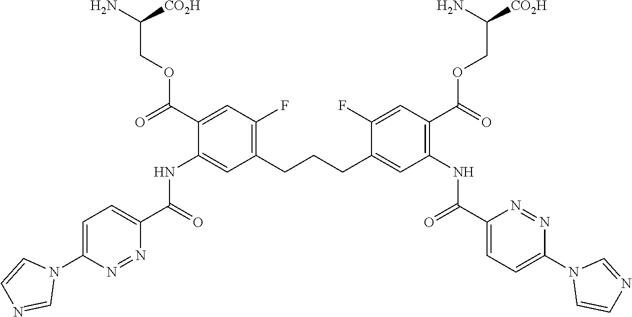 [SMILES: N[C@H](COC(=O)c1cc(F)c(CCCc2cc(NC(=O)c3ccc(-n4ccnc4)nn3)c(C(=O)OC[C@@H](N)C(=O)O)cc2F)cc1NC(=O)c1ccc(-n2ccnc2)nn1)C(=O)O] | |
| 233 | 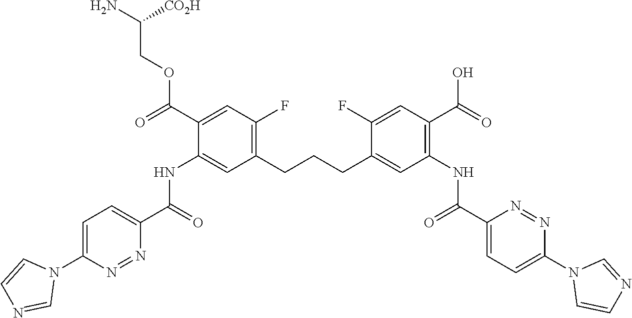 [SMILES: N[C@@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] | |
| 234 | 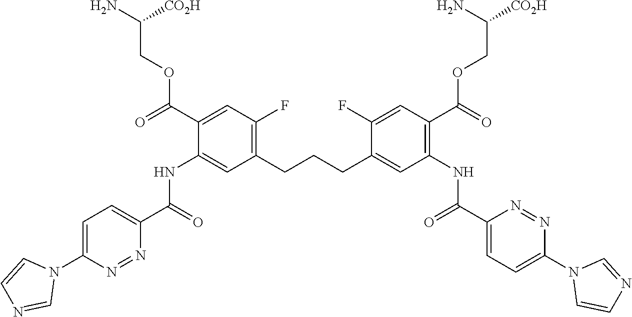 [SMILES: N[C@@H](COC(=O)c1cc(F)c(CCCc2cc(NC(=O)c3ccc(-n4ccnc4)nn3)c(C(=O)OC[C@H](N)C(=O)O)cc2F)cc1NC(=O)c1ccc(-n2ccnc2)nn1)C(=O)O] | |
| 235 | 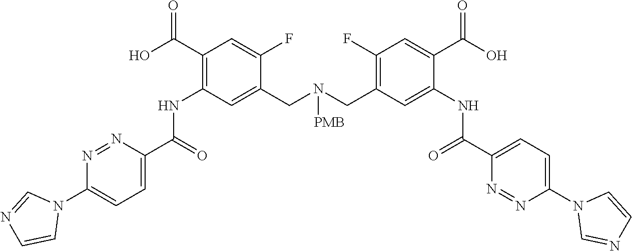 | MS-ESI: m/z 814.7 observed [M − H]⁻ (PMB = para-methoxybenzyl) |
| 236 | 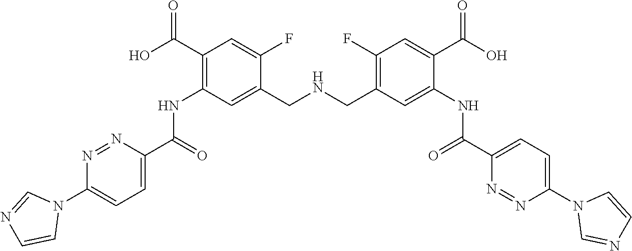 [SMILES: O=C(Nc1cc(CNCc2cc(NC(=O)c3ccc(-n4ccnc4)nn3)c(C(=O)O)cc2F)c(F)cc1C(=O)O)c1ccc(-n2ccnc2)nn1] | MS-ESI: m/z 696.51 observed [M + H]⁺ |
| 237 | 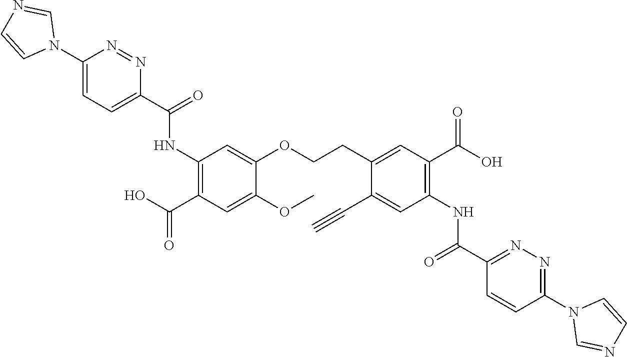 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1OC] | MS-ESI: m/z 713.2 observed [M − H]⁻ |
| 238 | 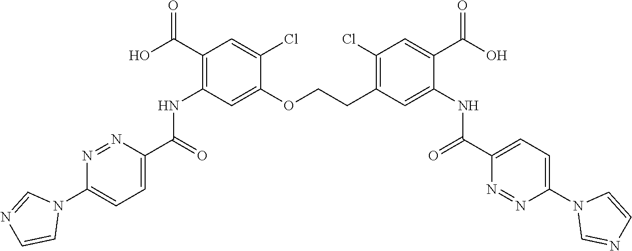 [SMILES: O=C(Nc1cc(CCOc2cc(NC(=O)c3ccc(-n4ccnc4)nn3)c(C(=O)O)cc2Cl)c(Cl)cc1C(=O)O)c1ccc(-n2ccnc2)nn1] | MS-ESI: m/z 729.2 observed [M + H]⁺ |
| 239 | 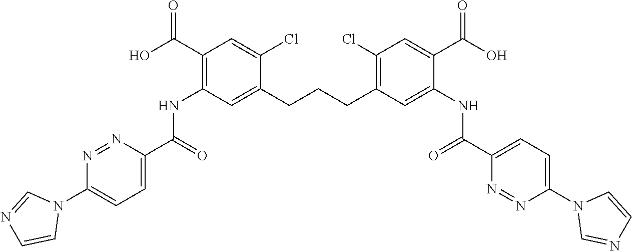 [SMILES: O=C(Nc1cc(CCCc2cc(NC(=O)c3ccc(-n4ccnc4)nn3)c(C(=O)O)cc2Cl)c(Cl)cc1C(=O)O)c1ccc(-n2ccnc2)nn1] | MS-ESI: m/z 727.2 observed [M + H]⁺ |

Pharmaceutical Composition

The present disclosure provides in another embodiment a pharmaceutical composition comprising a compound or pharmaceutically acceptable salt thereof as described herein in combination with a pharmaceutically acceptable carrier or excipient.

Compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the compound or a pharmaceutically acceptable salt thereof.

For tablet compositions, the compound or a pharmaceutically acceptable salt thereof in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the compound or a pharmaceutically acceptable salt thereof is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compound or a pharmaceutically acceptable salt thereof in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the compound or a pharmaceutically acceptable salt thereof in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compound the compound or a pharmaceutically acceptable salt thereof may also be administered in the form of suppositories for rectal administration. These compositions can be prepared by mixing the compound with a suitable non-irritating excipient which is solid at ordinary temperatures but liquid at the rectal temperature and will therefore melt in the rectum to release the compound. Exemplary excipients include cocoa butter and polyethylene glycols.

Compositions for parenteral administrations are administered in a sterile medium. Depending on the vehicle used and concentration the concentration of the compound or a pharmaceutically acceptable salt thereof in the formulation, the parenteral formulation can either be a suspension or a solution containing dissolved compound. Adjuvants such as local anesthetics, preservatives and buffering agents can also be added to parenteral compositions.

Methods of Use

The present disclosure also provides in an embodiment a method of stimulating expression of interferon genes in a human patient. The method comprises administering to the patient a therapeutically effective amount of a compound or pharmaceutically acceptable salt thereof as described herein. In accordance with exemplary data described herein, the compounds of the present disclosure are useful in the method as agonists of STING. In an embodiment, administration is carried out in vivo or, per another embodiment, in vitro.

In another embodiment, the present disclosure provides a method of treating a tumor in a patient. The method comprises administering to the patient a therapeutically effective amount of a compound or pharmaceutically acceptable salt thereof as disclosed herein. In this context, the role of STING, and specifically the activation thereof, already is acknowledged in antitumor immunity, such as in publications 1-4 below:
[1a] Corrales L, Glickman L H, McWhirter S M, Kanne D B, Sivick K E, Katibah G E, Woo S R, Lemmens E, Banda T, Leong J J, Metchette K, Dubensky T W Jr, Gajewski T F. (2015) Direct Activation of STING in the Tumor Microenvironment Leads to Potent and Systemic Tumor Regression and Immunity. Cell Rep. 11: 1018-30.
[1b] Chin, E. et al. (2020) Antitumor activity of a systemic STING-activating non-nucleotide cGAMP mimetic, Science. 369: 6506.
[1c] Pan, B. et al. (2020) An orally available non-nucleotide STING agonist with antitumor activity, Science. 369: 6506.
[1d] Ramanjulu. J. et al. (2018) Design of amidobenzimidazole STING receptor agonists with systemic activity, Nature. 564: 7736.
[2] Deng, L. et al. (2014) STING-Dependent Cytosolic DNA Sensing Promotes Radiation-Induced Type I Interferon-Dependent Antitumor Immunity in Immunogenic Tumors, Immunity. 41: 843.
[3] Corrales L. Matson V, Flood B. Spranger S, Gajewski T F. (2017) Innate immune signaling and regulation in cancer immunotherapy. Cell Res. 27: 96-108.
[4] Corrales L, McWhirter S M, Dubensky T W Jr, Gajewski T F. (2016) The host STING pathway at the interface of cancer and immunity. J Clin Invest. 126: 2404-11.

In various embodiments, the methods described herein entail combination therapies. For example, in embodiments optionally in combination with any other embodiment described here, a method further comprises administering an immune-checkpoint targeting drug. In other embodiments, a compound described herein is administered in coordination with anti-tumor therapies that entail ionizing radiation and/or and existing chemotherapeutic approaches, such as DNA-damage-based chemotherapies. The STING agonists of the present disclosure can complement, enhance efficacy of, and/or potentiate the harmful effects of these known therapeutic approaches. Evidence illustrating the critical role of STING-dependent micronuclei-mediated tumor clearance using these approaches resides, for example, in publications 5-8 below:
[5] Mackenzie, K. F., et all, (2017), cGAS surveillance of micronuclei links genome instability to innate immunity. Nature, 548, 461.
[6] Wang, W. et al., (2016). Effector T Cells Abrogate Stroma-Mediated Chemoresistance in Ovarian Cancer, Cell, 165, 1092-1105.
[7] Charlotte E. Ariyan, et al., Jan. 16, 2018; DOI: 10.1158/2326-6066, Robust antitumor responses result from local chemotherapy and CTLA-4 blockade, cancerimmunolres.aacrjournals.org on Jan. 31, 2018.
[8] Chung Kil Song, et al., www.moleculartherapy.org vol. 15 no. 8 Aug. 2007, Chemotherapy Enhances CD8+ T Cell-mediated Antitumor Immunity Induced by Vaccination With Vaccinia Virus.

Compounds of the present disclosure are also useful in the methods described herein, further comprising the administration of an effective dose of an immune-checkpoint targeting drug. For example, in various embodiments, the immune-checkpoint targeting drug is an anti-PD-L1 antibody, anti-PD-1 antibody, anti-CTLA-4 antibody, or an anti-4-1BB antibody as illustrated in publications 9-11 below:
[9] Ager, C R, et al., (2017) Cancer Immunol Res; 5(8), 676.
[10] Fu, J. et al. (2015) Sci Transl Med. 2015 Apr. 15; 7(283): 283ra52. doi:10.1126/scitranslmed.aaa4306.
[11] Wang, H., et al. (2017) PNAS, Feb. 14, 2017, vol. 114, no. 7, 1637-1642.

### EXAMPLES

The following non-limiting examples are additional embodiments for illustrating the present disclosure.

Compounds of the present disclosure are prepared according to the following procedures in conjunction with ordinary knowledge and skill in organic synthesis, substituting appropriate reagents as apparent to the practitioner.

Experimental Procedures

Abbreviations. The following abbreviations are used: tetrahydrofuran (THF), dichloromethane (DCM), N,N-dimethylformamide (DMF), dimethylacetamide (DMA), dimethylsulfoxide (DMSO), trifluoroacetic acid (TFA), triethylamine (TEA), diisopropylethylamine (DIPEA), (1-Cyano-2-ethoxy-2-oxoethylidenaminooxy)dimethylamino-morpholino-carbenium hexafluorophosphate (COMU), 1-[bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide (HATU), (2-Biphenyl)dicyclohexylphosphine (CyJohnPhos), 1-propanephosphonic anhydride (T3P).

General Examples for the Preparation of Compounds of the Present disclosure. The starting materials and intermediates for the compounds of this present disclosure are prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as Scifinder (www.cas.org) or Reaxys (www.reaxys.com).

Part I: Preparation of Intermediates

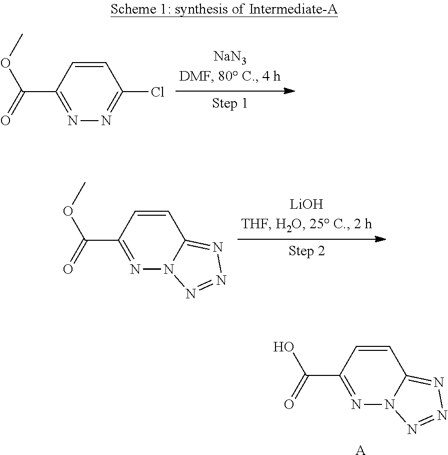

Step 1: Synthesis of methyl tetrazolo[1,5-b]pyridazine-6-carboxylate: To a solution of methyl 6-chloropyridazine-3-carboxylate (2.00 g, 11.6 mmol, 1.00 eq.) in DMF (10 mL) was added NaN₃ (2.26 g, 34.8 mmol, 3.00 eq.). The mixture was stirred at 80° C. for 4 hours. The residue was diluted with water (20 mL) and extracted with ethyl acetate (25 mL×3). The combined organic layers were washed with water (25 mL×3) and brine (25 mL×2), dried over anhydrous Na₂SO₄, filtered and concentrated under reduced pressure to give a residue. The residue was purified by column chromatography to give compound methyl tetrazolo[1,5-b]pyridazine-6-carboxylate (900 mg, 5.02 mmol, 43% yield, 99% purity) as a white solid. ¹H-NMR (400 MHz, DMSO-d6) δ 8.95 (d, J=9.6 Hz, 1H), 8.25 (d, J=9.2 Hz, 1H), 4.03 (s, 3H).

Step 2: Synthesis of tetrazolo[1,5-b]pyridazine-6-carboxylic acid (A): To a solution of methyl tetrazolo[1,5-b]pyridazine-6-carboxylate (900 mg, 5.02 mmol, 1.00 eq.) in THF (4 mL) was added a solution of LiOH·H₂O (632 mg, 15.1 mmol, 3.00 eq.) in H₂O (4 mL). After stirring at 25° C. for 1 hour, the mixture was neutralized with 6 M HCl. The precipitate was filtered, and the filter cake was dried under reduced pressure to give intermediate A (700 mg, 4.24 mmol, 84% yield, 99% purity) as a white solid. ¹H NMR (400 MHz, DMSO-d6) δ 14.69 (s, 1H), 8.91 (d, J=9.6 Hz, 1H), 8.222 (d, J=9.2 Hz, 1H).

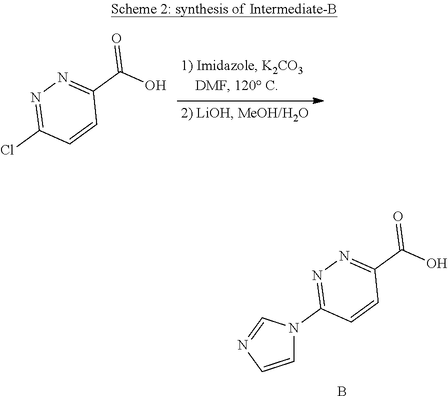

Synthesis of 6-(1H-imidazol-1-yl)pyridazine-3-carboxylic acid (B): To a suspension of methyl 6-chloropyridazine-3-carboxylate (1 g, 5.8 mmol) and imidazole (0.4 g, 5.8 mmol) in dry DMF (10 mL), was added K₂CO₃ (940 mg, 6.8 mmol) and the reaction mixture was stirred at 120° C. for 3 h. The reaction was monitored by LCMS. After completion of the reaction, a 2.5M aqueous solution of LiOH (2.8 mL, 6.96 mmol) was added to the reaction mixture and stirred at 60° C. for 1 h. The reaction was monitored by LCMS. After completion of the reaction, the reaction mixture was acidified with 1M HCl aqueous solution and the resulting precipitate was filtered and washed with water, to afford intermediate B (720 mg) as an off-white solid which was used in the next step without further purification. LC-MS (ESI+): m/z 191.0 [M+H]⁺.

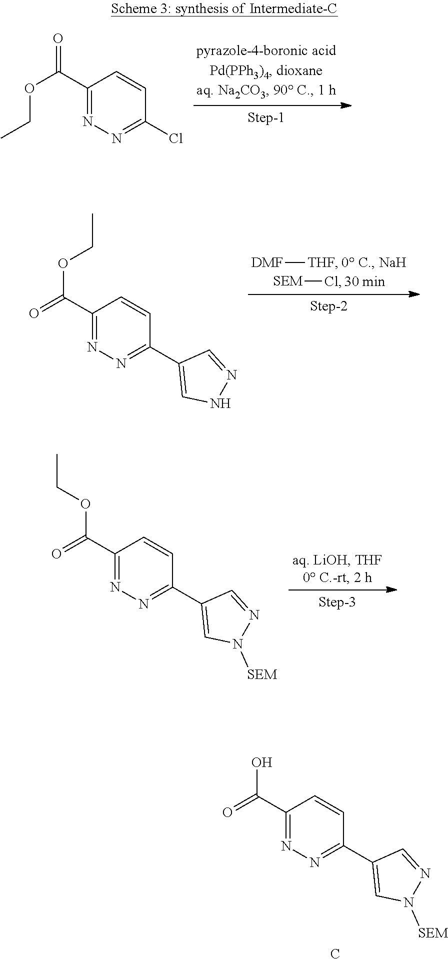

Step 1: Synthesis of ethyl 6-(1H-pyrazol-4-yl)pyridazine-3-carboxylate: Argon gas was purged through a solution of pyrazole-4-boronic acid (4.51 g, 40.31 mmol), Na₂CO₃ (7.1 g, 67.2 mmol) and ethyl 6-chloropyridazine-3-carboxylate (5 g, 26.88 mmol) in 1,4-dioxane (175 mL) and water (25 mL) for 10 mins before addition of Pd(PPh₃)₄ (1.55 g, 1.34 mmol). The reaction mixture was stirred at 90° C. for 1 h. After completion of the reaction, it was cooled to room temperature and diluted with EtOAc (250 mL). It was then washed with water (100 mL), brine (100 mL), dried over anhydrous Na₂SO₄, filtered and concentrated under reduced pressure. The crude material was purified by silica gel column chromatography over silica gel to afford 3.2 g of ethyl 6-(1H-pyrazol-4-yl)pyridazine-3-carboxylate as an off-white solid. LC-MS (ESI+): m/z; 219.0 [M+H]⁺.

Step 2: Synthesis of ethyl 6-(1-((2-(trimethylsilyl) ethoxy)methyl)-1H-pyrazol-4-yl)pyridazine-3-carboxylate: NaH (60% w/w) (0.422 g, 17.6 mmol) was added portion wise to a stirred solution of ethyl 6-(1H-pyrazol-4-yl)pyridazine-3-carboxylate (3.2 g, 14.67 mmol) in THF (64 mL) and DMF (30 mL) at 0° C. and stirred for 10 mins. To this, was added SEM-Cl (2.93 g, 17.61 mmol) and the reaction mixture was stirred at 0° C. for 30 min. It was then quenched with 10% citric acid solution and the solid thus obtained was filtered, washed with water (5 mL×2) and dried. The residue was purified by silica gel column chromatography using 0-5% Methanol in Dichloromethane as eluent to afford 2.65 g of ethyl 6-(1-((2-(trimethylsilyl)ethoxy)methyl)-1H-pyrazol-4-yl)pyridazine-3-carboxylate as an off-white solid. LC-MS (ESI+): m/z; 349.1 [M+H]⁺.

Step 3: Synthesis of 6-(1-((2-(trimethylsilyl)ethoxy)methyl)-1H-pyrazol-4-yl)pyridazine-3-carboxylic acid (C): To a solution of ethyl 6-(1-((2-(trimethylsilyl) ethoxy) methyl)-1H-pyrazol-4-yl)pyridazine-3-carboxylate (2.65 g, 7.61 mmol) in THF (9 mL) was added an aqueous solution of lithium hydroxide monohydrate (0.382 g, 9.13 mmol, in 3 mL water) at 0° C. and the reaction mixture was stirred at room temperature for 2 h. After completion of the reaction, the reaction mixture was diluted with water (10 mL) and washed with EtOAc (30 mL×2). The aqueous layer was acidified using 2N HCl solution (pH=4) and the solid was filtered, washed with water (2 mL×2) and dried to afford 1.1 g of intermediate C as an off-white solid. ¹H NMR (400 MHz, DMSO-d₆) δ 13.62 (s, 1H), 8.78 (s, 1H), 8.33 (s, 1H), 8.18-8.13 (m, 2H), 5.51 (s, 2H), 3.61 (t, J=8.0 Hz, 2H), 0.87 (d, J=8.0 Hz, 2H), 0.04 (s, 9H). LC-MS (ESI+): m/z 321.0 [M+H]⁺.

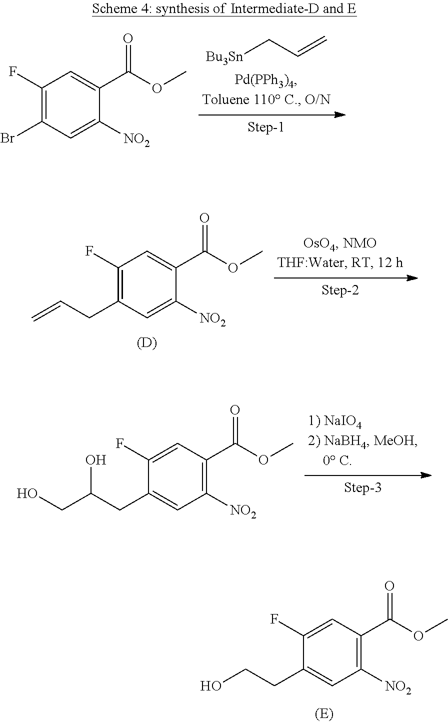

Step 1: Synthesis of methyl 4-allyl-5-fluoro-2-nitrobenzoate (D): To a stirred solution of methyl 4-bromo-5-fluoro-2-nitrobenzoate (20 g, 71.92 mmol, 1 eq.) in Toluene (200 mL) was added allyltributylstannane (30.96 g, 93.50 mmol, 1.3 eq.) at rt (room temperature). The reaction mixture was purged with Argon gas for 20 min. To this, Pd(PPh₃)₄ (1.67 g, 1.44 mmol, 0.02 eq.) was added at rt and stirred at 110° C. overnight. After completion of the reaction, reaction mixture was cooled at rt and diluted with cold water (200 mL). The resultant aqueous solution was stirred with 1M aqueous solution of potassium fluoride (KF) for 30 min. and extracted with Ethyl Acetate (2×300 mL). The combined organic layers were dried over anhydrous Na₂SO₄ and evaporated to get crude product. The crude material was purified through silica gel column chromatography using 2-3% Ethyl Acetate in Hexane to get pure Intermediate D (15.1 g, 87.76%) as a brown liquid. ¹H-NMR (400 MHz, DMSO-d₆) δ 7.87 (d, J=6 Hz, 1H), 7.41 (d, J=8.4 Hz, 1H), 6.05-5.95 (m, 1H), 5.27-5.18 (m, 2H), 3.99 (s, 3H), 3.53 (d, J=6.4, 2H).

Step 2: Synthesis of methyl 4-(2,3-dihydroxypropyl)-5-fluoro-2-nitrobenzoate: To a solution of intermediate D (5 g, 20.92 mmol, 1 eq.) in THF (100 mL) and Water (20 mL) was added 0.02 M Osmium tetroxide (OsO₄) solution in tert-Butyl alcohol (21 mL, 0.42 mmol, 0.02 eq.) and N-Methylmorpholine N-oxide (NMO) (2.45 g, 20.92 mmol, 1 eq.) at rt. The reaction mixture was stirred at rt for 12 h and monitored by TLC. After completion of the reaction, reaction mixture was diluted with cold water (300 mL). The aqueous layer was extracted with Ethyl Acetate (2×150 mL). The combined organic layer was dried over anhydrous Na₂SO₄ and evaporated to get crude product. The crude material was purified through silica gel column chromatography using 4% MeOH in DCM as eluent to get pure methyl 4-(2,3-dihydroxypropyl)-5-fluoro-2-nitrobenzoate (3.1 g, 54.28% yield) as a solid. ¹H-NMR (400 MHz, DMSO-d₆) δ 8.12 (d, J=6.5 Hz, 1H), 7.72 (d, J=9.6 Hz, 1H), 4.85 (d, 1H), 4.75 (t, 1H), 3.91 (s, 3H), 3.68 (m, 1H), 3.48 (m, 1H); 3.33 (m, 1H); 2.96 (m, 1H); 2.66 (m, 1H).

Step 3: Synthesis of methyl 5-fluoro-4-(2-hydroxyethyl)-2-nitrobenzoate (E): To a solution of Intermediate C (3.1 g, 11.35 mmol, 1 eq.) in MeOH (90 mL) and Water (90 mL) was added Sodium periodate (2.91 g, 13.62 mmol, 1.2 eq). The reaction mixture was stirred at 0° C. for 1 h and monitored by TLC. Then, Sodium borohydride (0.52 g, 13.62 mmol, 1.2 eq) was added and stirred at rt for 1 h. After completion of the reaction, the reaction mass was diluted with cold water (300 mL). The aqueous solution was extracted with 10% MeOH in DCM (2×150 mL) and the combined organic layers were dried over Na₂SO₄ and evaporated to get crude product. The crude material was purified through silica gel column chromatography using 2-3% MeOH in DCM as a gradient to get pure Intermediate E (2.7 g, 97.85%) as a solid. ¹H-NMR (400 MHz, DMSO-d₆) δ 8.18 (d, J=6.4 Hz, 1H), 7.76 (d, J=6.4 Hz, 1H), 5.75 (m, 1H), 4.66 (d, J=6.4 Hz, 2H), 3.86 (t, J=11.2 Hz, 2H), 3.38 (s, 3H).

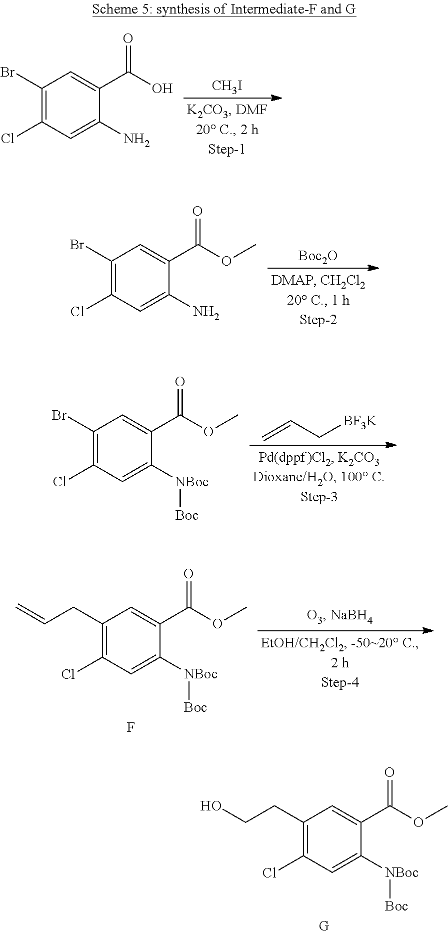

Step 1: Synthesis of methyl 2-amino-5-bromo-4-chlorobenzoate: To a solution of 2-amino-5-bromo-4-chloro-benzoic acid (15 g, 58.0 mmol, 97% purity, 1 eq) and CH₃I (16.4 g, 116 mmol, 7.23 mL, 2 eq) in DMF (200 mL) was added K₂CO₃ (16.0 g, 116 mmol, 2 eq). The mixture was stirred at 25° C. for 3 hrs. The reaction mixture was filtered and slowly poured into the water to filter out the solids, then washed with Ethyl Acetate (100 mL) and brine (50 mL×3), dried over anhydrous Na₂SO₄, filtered and concentrated under reduced pressure to give methyl 2-amino-5-bromo-4-chlorobenzoate (22.2 g, crude) as a yellow solid. The crude product was used for the next step without further purification. MS-ESI: m/z 265.9 observed [M+H]⁺.

Step 2: Synthesis of methyl 2-amino-5-bromo-4-chlorobenzoate: To a solution of methyl 2-amino-5-bromo-4-chloro-benzoate (22.2 g, 76.6 mmol, 1 eq) and Boc₂O (66.9 g, 306 mmol, 70.4 mL, 4 eq) in CH₂Cl₂ (200 mL) was added DMAP (9.36 g, 76.6 mmol, 1 eq). The mixture was stirred at 25° C. for 3 hrs. The reaction solution was quenched with water (100 mL) and extracted with Ethyl Acetate (200 mL×3), dried over anhydrous Na₂SO₄, filtered and concentrated under reduced pressure. The crude material was purified by flash silica gel chromatography using 0˜25% ethyl acetate/petroleum ether as a gradient to afford methyl 2-amino-5-bromo-4-chlorobenzoate (4.08 g, 8.81 mmol, 15% yield) as a white solid. ¹H NMR (400 MHz, DMSO-d₆) δ 8.20 (s, 1H), 7.84 (s, 1H), 3.80 (s, 3H), 1.33 (s, 18H).

Step 3: Synthesis of methyl 5-allyl-2-(bis(tert-butoxycarbonyl)amino)-4-chlorobenzoate (F): A mixture of methyl 2-amino-5-bromo-4-chlorobenzoate (4 g, 8.61 mmol, 1 eq), Potassium allyltrifluoroborate (2.55 g, 17.2 mmol, 2 eq), K₂CO₃ (3.57 g, 25.8 mmol, 3 eq), Pd(dppf)Cl₂ (629 mg, 0.860 mmol, 0.1 eq) in dioxane (60 mL) and water (6 mL) was degassed and purged with N₂ for 3 times, and then the mixture was stirred at 80° C. for 12 hrs in the atmosphere of N₂. The reaction mixture was partitioned between water (100 mL) and Ethyl Acetate (80 mL). The organic phase was separated, washed with brine (100 mL), dried over anhydrous Na₂SO₄, filtered and concentrated under reduced pressure to give a crude product. The crude material was purified by flash silica gel chromatography using 0˜5% Ethyl acetate/Petroleum ether as a gradient to afford intermediate F (1.28 g, 3.01 mmol, 34% yield) as a yellow oil. ¹H NMR (400 MHz, CDCl₃) δ 7.89 (s, 1H), 7.23 (s, 1H), 6.01-5.92 (m, 1H), 5.17-5.13 (m, 1H), 5.08-5.03 (m, 1H), 3.87 (s, 3H), 3.54 (d, J=6.4 Hz, 2H), 1.40 (s, 18H).

Step 4: Synthesis of methyl 2-(bis(tert-butoxycarbonyl)amino)-4-chloro-5-(2-hydroxyethyl) benzoate (G): A mixture of methyl 5-allyl-2-[bis(tert-butoxycarbonyl)amino]-4-chloro-benzoate (1.28 g, 3.01 mmol, 1 eq) in CH₂Cl₂ (20 mL) and EtOH (2 mL) was ozonolyzed with ozone (15 psi) at −50° C., then the mixture was warmed up to 20° C. and then NaBH₄ (227 mg, 6.01 mmol, 2 eq) was added to the mixture and the mixture was stirred at 20° C. for 2 hrs. The mixture was carefully acidified with aqueous 10% HCl (30 mL), concentrated under reduced pressure and extracted with Ethyl Acetate (30 mL×3). The combined organic phase was washed with brine (30 mL), dried over anhydrous sodium sulfate, filtered and concentrated under reduced pressure. The crude material was purified by flash silica gel chromatography using 0˜40% ethyl acetate/petroleum ether as a gradient to afford intermediate G (500 mg, 1.11 mmol, 37% yield, 95% purity) as a white solid. ¹H NMR (400 MHz, DMSO-d₆) δ=7.90 (s, 1H), 7.49 (s, 1H), 4.79 (t, J=5.2 Hz, 1H), 3.66-3.61 (m, 2H), 2.91 (t, J=6.4 Hz, 2H), 1.34 (s, 18H).

Part II: Preparation of Example Compounds

All compounds of the present disclosure were prepared using the procedures exemplified below.

### Example 1

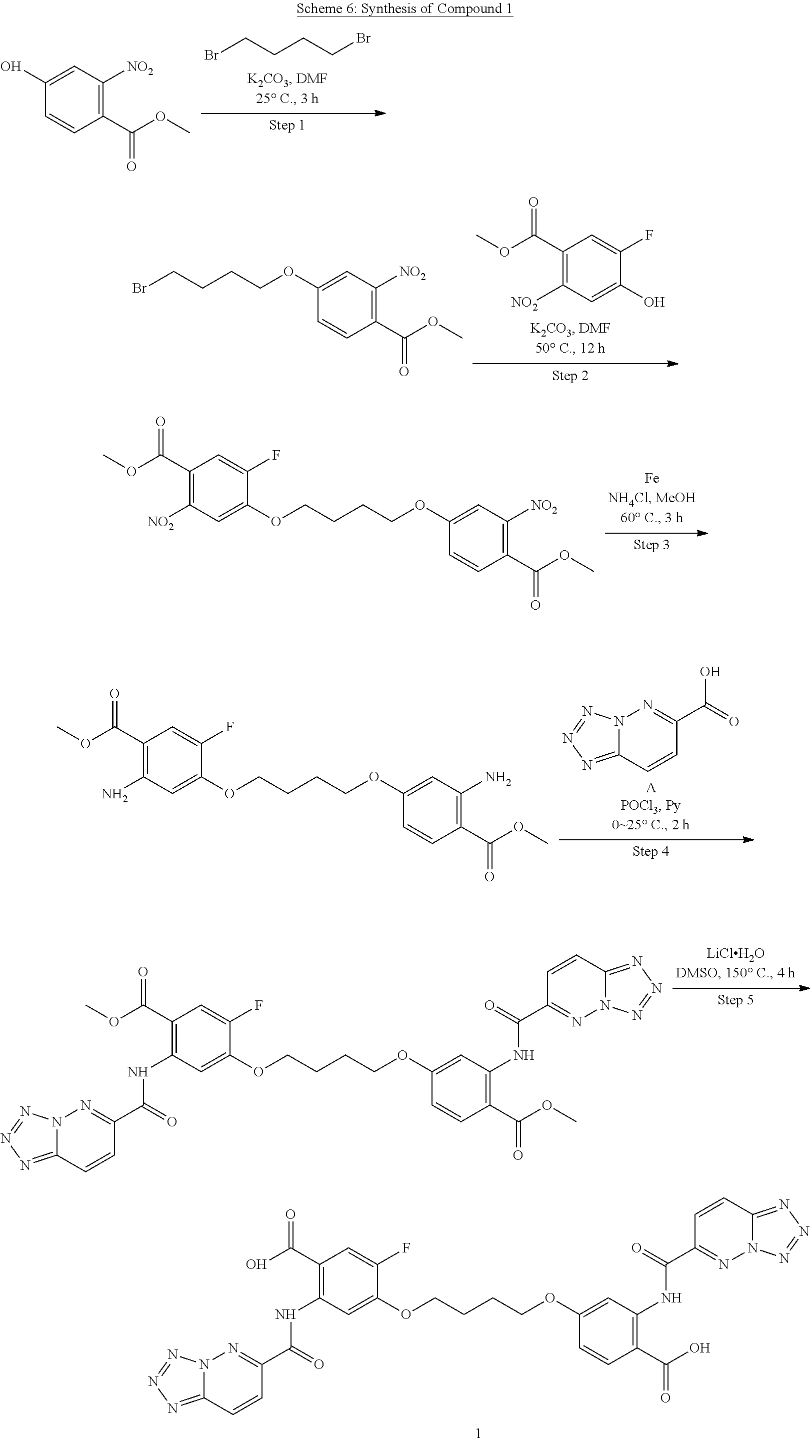

Step 1: Synthesis of methyl 4-(4-bromobutoxy)-2-nitrobenzoate: To a solution of methyl 4-hydroxy-2-nitro-benzoate (300 mg, 1.52 mmol, 1 eq.) and 1,4-dibromobutane (1.64 g, 7.61 mmol, 917 uL, 5 eq.) in DMF (10 mL) was added K₂CO₃ (630 mg, 4.57 mmol, 3 eq). Then the mixture was stirred at 25° C. for 3 hrs. The reaction mixture was diluted with Ethyl Acetate (10 mL) and washed with water (10 mL×3), then the combined organic layer was washed with brine (20 mL), dried over anhydrous Na₂SO₄, filtered and the filtrate was concentrated. The crude material was purified by silica gel column chromatography to give methyl 4-(4-bromobutoxy)-2-nitro-benzoate (400 mg, 1.2 mmol, 79% yield) as a white solid. ¹H NMR (400 MHz, CDCl₃) δ 7.79 (d, J=8.8 Hz, 1H), 7.24 (d, J=2.4 Hz, 1H), 7.10 (dd, J=8.8, 2.4 Hz, 1H), 4.10 (t, J=6.0 Hz, 2H), 3.89 (s, 3H), 3.50 (t, J=6.4 Hz, 2H), 2.13-2.06 (m, 2H), 2.04-1.96 (m, 2H).

Step 2: Synthesis of methyl 5-fluoro-4-(4-(4-(methoxycarbonyl)-3-nitrophenoxy)butoxy)-2-nitrobenzoate: To a solution of methyl 4-(4-bromobutoxy)-2-nitro-benzoate (400 mg, 1.2 mmol, 1 eq.) and methyl 5-fluoro-4-hydroxy-2-nitro-benzoate (259 mg, 1.2 mmol, 1 eq.) in DMF (6 mL) was added K₂CO₃ (499 mg, 3.61 mmol, 3 eq.) and the mixture was stirred at 50° C. for 12 hrs. After completion of the reaction, the reaction mixture was poured into Ethyl Acetate (10 mL), and then the mixture was washed with water (10 mL×3). The combined organic layer was washed with brine (20 mL), dried over anhydrous Na₂SO₄, filtered and the filtrate was concentrated. The crude material was purified by silica gel column chromatography to give methyl 5-fluoro-4-[4-(4-methoxycarbonyl-3-nitro-phenoxy)butoxy]-2-nitro-benzoate (380 mg, 0.814 mmol, 67% yield) as a yellow solid. ¹H NMR (400 MHz, DMSO-d₆) δ 7.89 (d, J=7.2 Hz, 1H), 7.86 (d, J=8.8 Hz, 1H), 7.80 (d, J=10.8 Hz, 1H), 7.54 (d, J=2.4 Hz, 1H), 7.31 (dd, J=8.8, 2.4 Hz, 1H), 4.30 (t, J=5.6 Hz, 2H), 4.21 (t, J=5.6 Hz, 2H), 3.82 (s, 3H), 3.80 (s, 3H), 1.93-1.91 (m, 4H).

Step 3: Synthesis of methyl 2-amino-4-(4-(3-amino-4-(methoxycarbonyl)phenoxy)butoxy)-5-fluorobenzoate: To a solution of methyl 5-fluoro-4-[4-(4-methoxycarbonyl-3-nitro-phenoxy)butoxy]-2-nitro-benzoate (380 mg, 0.814 mmol, 1 eq.) in MeOH (8 mL) was added NH₄Cl (436 mg, 8.15 mmol, 10 eq.) and Fe (227 mg, 4.07 mmol, 5 eq.), then the mixture was stirred at 60° C. for 3 hrs. After completion of the reaction, the reaction mixture was diluted with DCM (20 mL), filtered, and filtrate was concentrated under vacuum. The residue was purified by silica gel column chromatography to give methyl 2-amino-4-[4-(3-amino-4-methoxycarbonyl-phenoxy)butoxy]-5-fluoro-benzoate (220 mg, 0.541 mmol, 66% yield) as a yellow solid. ¹H NMR (400 MHz, CDCl₃) δ 7.80 (br d, J=8.8 Hz, 1H), 7.55 (d, J=12.4 Hz, 1H), 6.30-6.09 (m, 3H), 4.12-4.02 (m, 4H), 3.85 (s, 6H), 2.01-1.99 (m, 4H). MS-ESI: m/z 407.0 observed [M+H]⁺.

Step 4: Synthesis of methyl 5-fluoro-4-(4-(4-(methoxycarbonyl)-3-(tetrazolo[1,5-b]pyridazine-6-carboxamido)phenoxy)butoxy)-2-(tetrazolo[1,5-b]pyridazine-6-carboxamido)benzoate: To a solution of methyl 2-amino-4-[4-(3-amino-4-methoxycarbonyl-phenoxy)butoxy]-5-fluoro-benzoate (100 mg, 0.246 mmol, 1 eq.) and intermediate A (102 mg, 0.615 mmol, 2.5 eq.) in Pyridine (1 mL) was added POCl₃ (226 mg, 1.17 mmol, 137 uL, 6 eq.) at 0° C., then the mixture was stirred at 25° C. for 2 h. The reaction mixture was poured into water (20 mL), then the mixture was filtered, and the filter cake was collected. The crude product was triturated with water (2 mL) at 25° C. for 5 min to afford methyl 5-fluoro-4-[4-[4-methoxycarbonyl-3-(tetrazolo[1,5-b]pyridazine-6-carbonylamino)phenoxy]butoxy]-2-(tetrazolo[1,5-b]pyridazine-6-carbonylamino)benzoate (80 mg, 0.114 mmol, 46% yield) as yellow solid. ¹H NMR (400 MHz, DMSO-d₆) δ 12.95-12.84 (m, 1H), 12.77 (br s, 1H), 9.07-8.88 (m, 2H), 8.77-8.56 (m, 1H), 8.45-8.26 (m, 3H), 8.04 (br d, J=8.4 Hz, 1H), 7.78 (br d, J=11.2 Hz, 1H), 6.96-6.83 (m, 1H), 4.35-4.17 (m, 4H), 4.00-3.90 (m, 6H), 2.05-1.96 (m, 4H). MS-ESI: m/z 701.1 observed [M+H]⁺.

Step 5: Synthesis of 4-(4-(4-carboxy-3-(tetrazolo[1,5-b]pyridazine-6-carboxamido) phenoxy)butoxy)-5-fluoro-2-(tetrazolo[1,5-b]pyridazine-6-carboxamido)benzoic acid (1): To a solution of methyl 5-fluoro-4-[4-[4-methoxycarbonyl-3-(tetrazolo[1,5-b]pyridazine-6-carbonylamino)phenoxy]butoxy]-2-(tetrazolo[1,5-b]pyridazine-6-carbonylamino)benzoate (60 mg, 0.086 mmol, 1 eq) in DMSO (1 mL) was added LiCl·H₂O (130 mg, 2.06 mmol, 24 eq), then the mixture was stirred at 150° C. for 4 hrs. To the reaction mixture was added water (0.3 mL), then the mixture was filtered, and the filter cake was collected. The crude product was triturated with water (2 mL) at 25° C. for 5 min to afford compound 1 (43 mg, 0.064 mmol, 74% yield) as a yellow solid. ¹H NMR (400 MHz, DMSO-d₆) δ 13.71 (s, 2H), 8.97 (d, J=9.4 Hz, 2H), 8.64 (d, J=8.0 Hz, 1H), 8.49-8.27 (m, 3H), 8.04 (d, J=8.7 Hz, 1H), 7.77 (d, J=12.0 Hz, 1H), 6.87 (d, J=8.9 Hz, 1H), 4.37-4.15 (m, 4H), 2.14-1.90 (m, 4H). MS-ESI: m/z 673.2 observed [M+H]⁺

Procedures analogous to those for the synthesis of compound 1 were used for the synthesis of compounds 19, 25, 28, 30, 32, 49, 58, 69, 81, and 203.

### Example 2

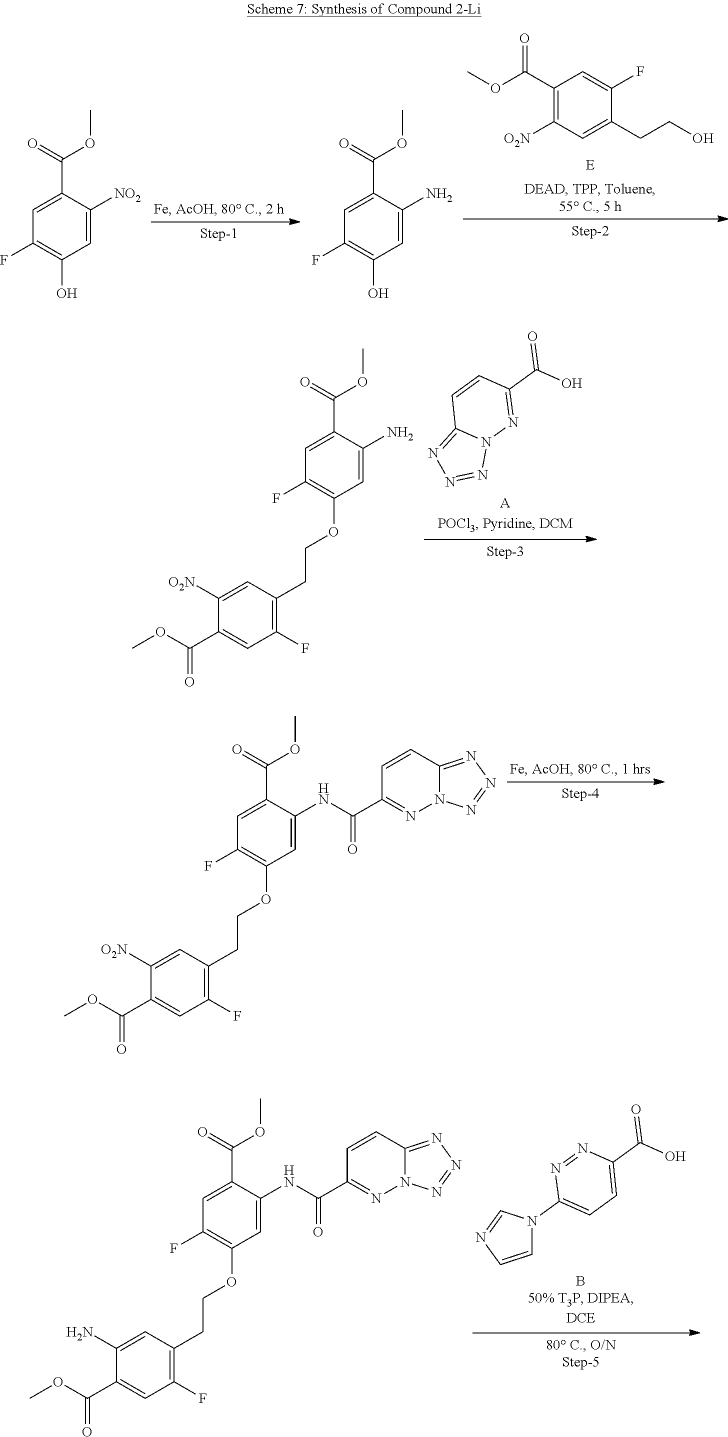

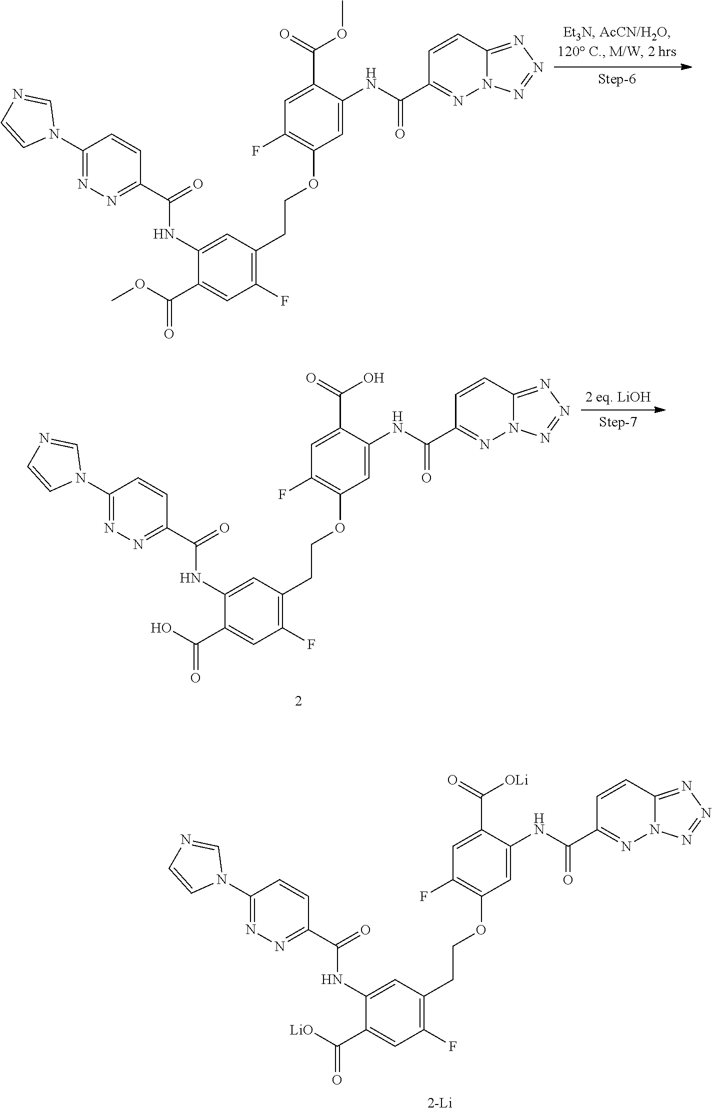

Step 1: Synthesis of methyl 2-amino-5-fluoro-4-hydroxybenzoate: To a stirred solution of methyl 5-fluoro-4-hydroxy-2-nitrobenzoate (2 g, 9.30 mmol, 1 eq.) in Acetic acid (20 mL) was added Fe powder (2.05 g, 37.19 mmol, 4 eq.) at rt and heated at 80° C. for 2 h. After completion of the reaction, reaction mixture was poured into cold water (300 mL). The resultant aqueous solution was extracted with Ethyl Acetate (2×300 mL). The combined organic layers were dried over anhydrous Na₂SO₄ and evaporated to get crude product. The crude material was purified through silica gel column chromatography using 15-20% Ethyl Acetate in Hexane as a gradient to get pure methyl 2-amino-5-fluoro-4-hydroxybenzoate (700 mg, 41% yield) as a solid. ¹H-NMR (400M1-z, DMSO-d₆) 10.54 (s, 11H), 7.36 (d, J=12.4 Hz, 1H), 6.53 (s, 2H), 6.30 (d, J=7.6 Hz, 11H), 3.73 (s, 3H).

Step 2: Synthesis of methyl 2-amino-5-fluoro-4-(2-fluoro-4-(methoxycarbonyl)-5-nitrophenethoxy)benzoate: To a solution of methyl 2-amino-5-fluoro-4-hydroxybenzoate (0.53 g, 2.88 mmol, 1 eq.) and Intermediate E (0.7 g, 2.88 mmol, 1 eq.) in toluene (7 mL) was added Ph₃P (1.51 g, 5.76 mmol, 2 eq.). To this, diethyl azodicarboxylate (DEAD) (1 g, 5.76 mmol, 2 eq.) was added at 55° C. and stirred at same temperature for 5 h. After completion of the reaction, reaction mixture was poured into cold water (500 mL). The resultant aqueous solution was extracted with Ethyl Acetate (2×200 mL). The combined organic layers were dried over anhydrous Na₂SO₄ and evaporated to get crude product. The crude material was purified through silica gel column chromatography using 20% Ethyl acetate in Hexane as eluent to get pure methyl 2-amino-5-fluoro-4-(2-fluoro-4-(methoxycarbonyl)-5-nitrophenethoxy)benzoate (650 mg, 55% yield) as a solid. ¹H-NMR (400 MHz, DMSO-d₆) δ 8.29 (d, J=6.0 Hz, 1H), 7.80 (d, J=9.1 Hz, 1H), 7.37 (d, J=12.4 Hz, 1H), 6.63 (s, 2H), 6.50 (d, J=7.6 Hz, 1H), 4.31 (t, J=6.3 Hz, 2H), 3.87 (s, 3H), 3.75 (s, 3H), 3.34-3.22 (m, 2H), MS-ESI: m/z 410.87 observed [M+H]⁺.

Step 3: Synthesis of methyl 5-fluoro-4-(2-(2-fluoro-4-(methoxycarbonyl)-5-(tetrazolo[1,5-b]pyridazine-6-carboxamido)phenoxy)ethyl)-2-nitrobenzoate: To a solution of methyl 2-amino-5-fluoro-4-(2-fluoro-4-(methoxycarbonyl)-5-nitrophenethoxy)benzoate (0.6 g, 1.46 mmol, 1 eq.) and intermediate A (0.6 g, 3.66 mmol, 2.5 eq.) in Pyridine (6 mL) was dropwise added POCl₃ (0.9 g, 0.55 mL, 5.85 mmol, 4 eq.) at 0° C. and stirred at rt for 1.5 h. After completion of the reaction, the reaction mixture was poured into cold water (50 mL) and stirred for 10 min. The solid was filtered and washed with 1N HCl solution to remove excess pyridine from solid. The crude material was purified through silica gel column chromatography using 2% Methanol in DCM as eluent to get pure methyl 5-fluoro-4-(2-(2-fluoro-4-(methoxycarbonyl)-5-(tetrazolo[1,5-b]pyridazine-6-carboxamido)phenoxy)ethyl)-2-nitrobenzoate (0.325 g, 40% yield) as a solid. MS-ESI: m/z 558.3 observed [M+H]⁺.

Step 4: Synthesis of methyl 2-amino-5-fluoro-4-(2-(2-fluoro-4-(methoxycarbonyl)-5-(tetrazolo[1,5-b]pyridazine-6-carboxamido)phenoxy)ethyl)benzoate: To a stirred solution of methyl 5-fluoro-4-(2-(2-fluoro-4-(methoxycarbonyl)-5-(tetrazolo[1,5-b]pyridazine-6-carboxamido)phenoxy)ethyl)-2-nitrobenzoate (0.325 g, 0.58 mmol, 1 eq.) in MeOH (5 mL) and THF (5 mL) was added Acetic acid (5 mL) and followed by Fe powder (0.19 g, 3.50 mmol, 6 eq.) at rt and heated at 85° C. for 1 h. After completion of the reaction, the reaction mixture was poured into cold water (50 mL) to get solid material. The resultant solid was filtered and dried well to get pure methyl 2-amino-5-fluoro-4-(2-(2-fluoro-4-(methoxycarbonyl)-5-(tetrazolo[1,5-b]pyridazine-6-carboxamido)phenoxy)ethyl) benzoate (250 mg, 81.30% yield) as a solid. ¹H NMR (400 MHz, DMSO-d %) S 3.12 (d, J=7.6 Hz, 2H), 3.79 (s, 3H), 3.97 (s, 3H), 4.43 (t, J=6.5 Hz, 2H), 6.57 (s, 2H), 6.82 (d, J=6.4 Hz, 1H), 7.41 (d, J=10.8 Hz, 1H), 7.88 (d, J=11.5 Hz, 1H), 8.41 (d, J=9.2 Hz, 1H), 8.64 (d, J=8.1 Hz, 1H), 9.06 (d, J=9.1 Hz, 1H), 12.83 (s, 1H); MS-ESI: m/z 527.9 observed [M+H].

Step 5: Synthesis of methyl 2-(6-(1H-imidazol-1-yl)pyridazine-3-carboxamido)-5-fluoro-4-(2-(2-fluoro-4-(methoxycarbonyl)-5-(tetrazolo[1,5-b]pyridazine-6carboxamido)phenoxy)ethyl)benzoate: To a stirred solution of intermediate B (0.11 g, 0.57 mmol, 1.2 eq.) in DCE (5 mL) was added DIPEA (0.43 g, 0.58 mL, 3.32 mmol, 7 eq.) and 50% solution of T₃P (in ethyl acetate) (1.5 mL, 2.37 mmol, 5 eq.) at rt. To this, methyl 2-amino-5-fluoro-4-(2-(2-fluoro-4-(methoxycarbonyl)-5-(tetrazolo[1,5-b]pyridazine-6-carboxamido)phenoxy)ethyl)benzoate (0.25 g, 0.47 mmol, 1 eq.) was added. The reaction mixture was heated at 80-90° C. overnight. After completion of the reaction, the reaction mixture was directly concentrated under vacuum. The crude material was purify by silica gel column chromatography using 2-3% MeOH in DCM as eluent to get pure desired product (0.185 g, 56% yield). ¹H NMR (400 MHz, DMSO-d₆) δ 3.19 (s, 2H), 3.96 (s, 61H), 4.54 (s, 2H), 7.29 (s, 1H), 7.85 (t, J=11.2 Hz, 2H), 8.24 (s, 1H), 8.39 (d, J=9.6 Hz, 1H), 8.51 (d, J=18.3 Hz, 2H), 8.64 (d, J=7.9 Hz, 1H), 8.84 (s, 1H), 8.95 (s, 1H), 9.04 (d, J=9.6 Hz, 1H), 12.81 (s, 1H), 12.90 (s, 1H); MS-ESI: m/z 700.2 observed [M+H].

Step 6: Synthesis of 2-(6-(1H-imidazol-1-yl)pyridazine-3-carboxamido)-4-(2-(4-carboxy-2-fluoro-5-(tetrazolo[1,5-b]pyridazine-6-carboxamido)phenoxy)ethyl)-5-fluorobenzoic acid (2): To a solution of methyl 2-(6-(1H-imidazol-1-yl)pyridazine-3-carboxamido)-5-fluoro-4-(2-(2-fluoro-4-(methoxycarbonyl)-5-(tetrazolo[1,5-b]pyridazine-6carboxamido)phenoxy)ethyl)benzoate (0.185 g, 0.26 mmol, 1 eq.) in ACN (5 mL) and Water (5 mL) was added TEA (0.27 g, 0.37 mL, 2.64 mmol, 10 eq.) at rt. The reaction mixture was stirred in microwave at 120° C. for 2 h. After completion of the reaction, the reaction mixture was concentrated under vacuum. The crude material was purified by Prep-HPLC to get compound 2 (110 mg, 62% yield). MS-ESI: m/z 672.2 observed [M+H]+

Step 7: Synthesis of lithium 2-(6-(1H-imidazol-1-yl)pyridazine-3-carboxamido)-4-(2-(4-carboxylato-2-fluoro-5-(tetrazolo[1,5-b]pyridazine-6-carboxamido)phenoxy)ethyl)-5-fluorobenzoate (2-Li): To a suspension of compound 2 (110 mg, 0.16 mmol, 1 eq.) in water (6 mL) was added LiOH·H₂O (13.8 mg, 0.33 mmol, 2 eq.). The resultant clear solution was then filtered to remove any insoluble particles and lyophilized to obtain 2-Li (100 mg, 91% yield). ¹H NMR (400 MHz, DMSO) δ 16.69 (s, 1H), 15.77 (s, 1H), 8.95 (d, J=9.6 Hz, 1H), 8.85 (d, J=7.2 Hz, 1H), 8.80 (s, 1H), 8.64 (d, J=8.0 Hz, 1H), 8.47 (d, J=8.8 Hz, 1H), 8.41 (d, J=9.2 Hz, 1H), 8.36 (d, J=9.6 Hz, 1H), 8.21 (s, 1H), 7.77 (d, J=11.6 Hz, 1H), 7.27 (s, 1H), 4.35 (t, J=6.8 Hz, 2H), 3.21 (t, J=6.0 Hz, 2H). MS-ESI: m/z 672.14 observed [M+H]⁺.

Procedures analogous to those for the synthesis of compound 2 were used for the synthesis of compounds such as 20, 22, 67, 97-100, 24, 63, 44, 60, 196, 62, 211-214, 64, 72-77, 82, 85-89, 126, 83, 91, 92, 95, 57, 102, 104-107, 109-118, 135-137, 158, 159, 184, 192, 205, 207, and 218.

### Example 3

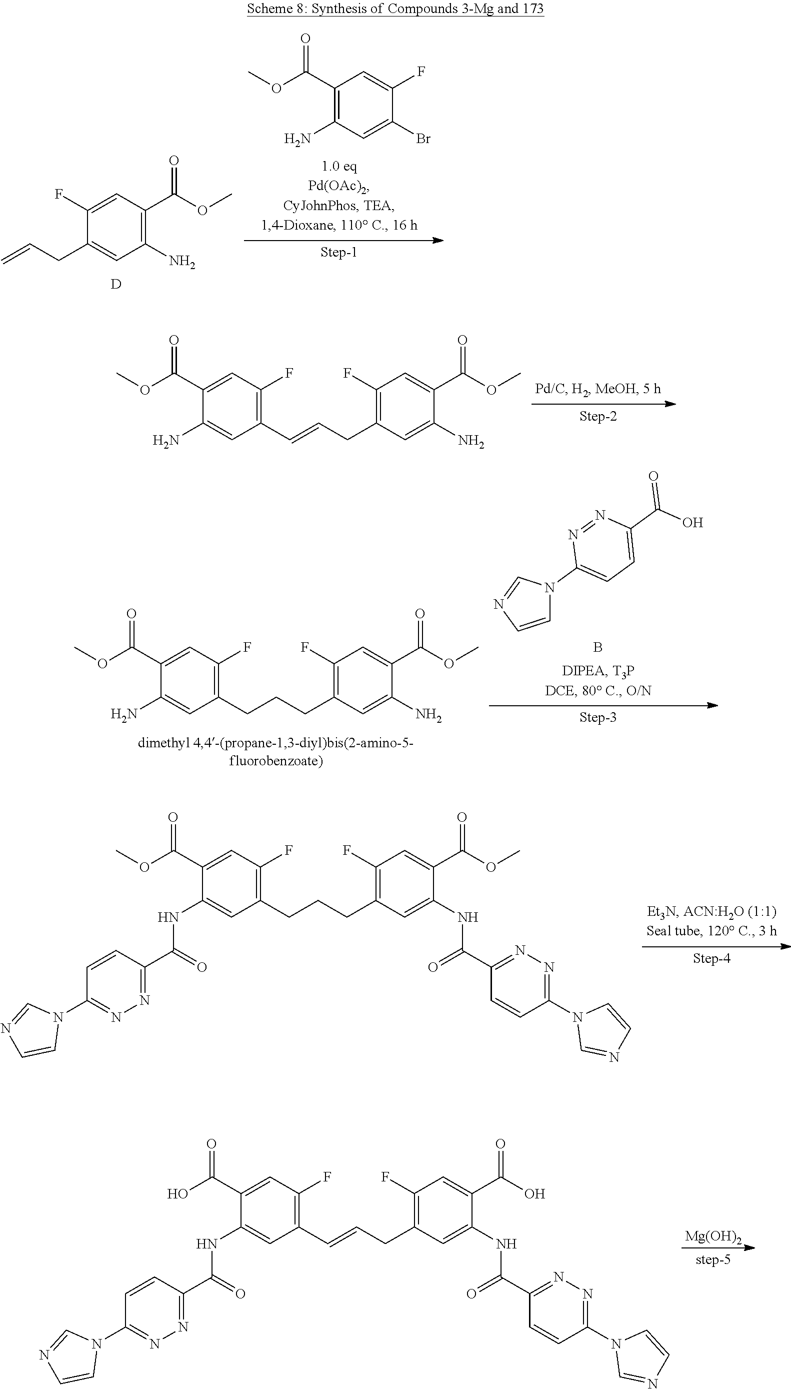

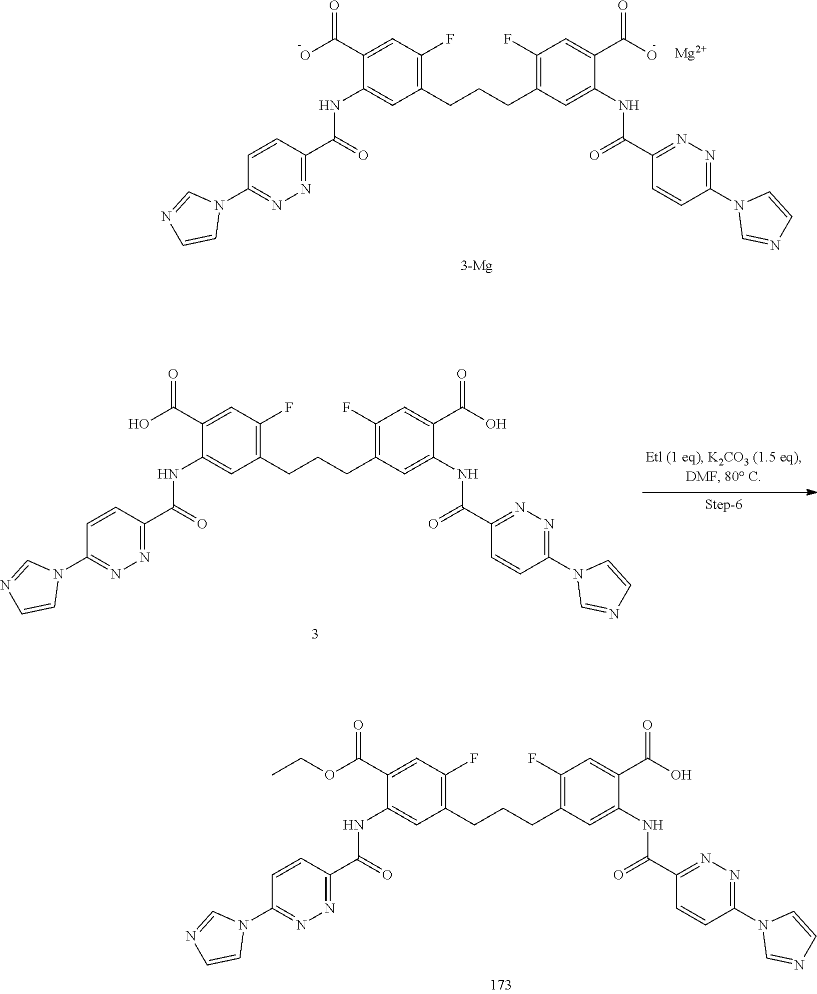 [SMILES: O=C(Nc1cc(CCCc2cc(NC(=O)c3ccc(-n4ccnc4)nn3)c(C(=O)O)cc2F)c(F)cc1C(=O)O)c1ccc(-n2ccnc2)nn1 ; [Mg+2] ; O=C(Nc1cc(CCCc2cc(NC(=O)c3ccc(-n4ccnc4)nn3)c(C(=O)O)cc2F)c(F)cc1C(=O)O)c1ccc(-n2ccnc2)nn1 ; CCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1]

Step 1: Synthesis of dimethyl 4,4′-(prop-1-ene-1,3-diyl)(E)-bis(2-amino-5-fluoro-benzoate): To a solution of intermediate D (8 g, 38.23 mmol, 1 eq.) and methyl 2-amino-4-bromo-5-fluorobenzoate (9.48 g, 38.23 mmol, 1 eq.) in 1,4 Dioxane (80 mL) was added TEA (13.43 ml, 95.50 mmol, 2.5 eq.) at rt. The reaction mixture was purged with Argon gas for 30 min. To this. Pd(OAc)₂ (0.43 g, 1.91 mmol, 0.05 eq,) and CyJohnPhos (1.34 g, 3.82 mmol, 0.1 eq.) was added at rt and the resultant mixture was stirred at 110° C. for 16 h. After completion of the reaction, the reaction mixture was cooled at rt and diluted with cold water (750 mL). The aqueous layer was extracted with Ethyl acetate (3×500 mL) and the combined organic layers were dried over anhydrous Na₂SO₄ and evaporated to get crude product. The crude material was purified through silica gel column chromatography using 15% Ethyl acetate in Hexanes as eluent to get pure dimethyl 4,4′-(prop-1-ene-1,3-diyl)(E)-bis(2-amino-5-fluoro-benzoate) (3.8 g, 26.41% yield) as a solid. ¹H NMR (400 MHz, DMSO-d₆) δ 7.41-7.38 (m, 2H), 6.96 (d, J=6.7 Hz, 1H), 6.72 (d, J=6.6 Hz, 1H), 6.57-6.45 (m, 6H), 3.79 (s, 6H), 3.54 (d, J=5.8 Hz, 2H). MS-ESI: m/z 377.0 observed [M+H]⁺.

Step 2: Synthesis of dimethyl 4,4′-(propane-1,3-diyl)bis(2-amino-5-fluorobenzoate): To a solution of dimethyl 4,4′-(prop-1-ene-1,3-diyl)(E)-bis(2-amino-5-fluoro-benzoate) (3.8 g, 10.09 mmol, 1 eq.) in MeOH (60 mL) and THF (60 mL) was added 10% Pd/C catalyst with 50% moist (1.9 g) at rt. The reaction mixture was purged with hydrogen gas for 5 h. After completion of the reaction, the reaction mixture was filtered on Celite bed and washed with 10% MeOH in DCM. The filtrate was concentrated under vacuum to get crude dimethyl 4,4′-(propane-1,3-diyl)bis(2-amino-5-fluorobenzoate) (3.6 g, 94.23%) which was used in next step without further purification. ¹H NMR (400 MHz, DMSO-d₆) δ 7.36 (d, J=11.0 Hz, 2H), 6.69 (d, J=6.7 Hz, 2H), 6.51 (s, 4H), 3.79 (s, 6H), 2.58 (t, J=7.7 Hz, 4H), 1.83-1.79 (m, 2H). MS-ESI: m/z 379.0 observed [M+H]⁺.

Step 3: Synthesis of dimethyl 4,4′-(propane-1,3-diyl)bis(2-(6-(1H-imidazol-1-yl)pyridazine-3-carboxamido)-5-fluorobenzoate): To a stirred solution of intermediate B (0.55 g, 2.91 mmol, 2.2 eq.) in DCE (7 ml) was added 50% solution of T₃P (in ethyl acetate) (5.04 mL, 7.93 mmol, 6 eq.) and DIPEA (1.84 ml, 10.57 mmol, 8 eq.) at it. To this, dimethyl 4,4′-(propane-1,3-diyl)bis(2-amino-5-fluorobenzoate) (0.5 g, 1.32 mmol, 1 eq.) was added at rt. The reaction mixture was heated at 80-90° C. overnight. After completion of the reaction, the reaction mixture was directly concentrated under reduced pressure to get crude material. To this, cold Sat. NaHCO₃ solution was added and stirred at rt for 15 min. The resulting precipitate were collected by filtration, washed with water and dried to get brown solid which was further purified by trituration using Methanol (2×10 ml) and Ethyl acetate (10 ml) to get pure dimethyl 4,4′-(propane-1,3-diyl)bis(2-(6-(1H-imidazol-1-yl)pyridazine-3-carboxamido)-5-fluorobenzoate) (0.75 g, 79% yield) as a solid. MS-ESI: m/z 723.2 observed [M+H]⁺.

Step 4: Synthesis of 4,4′-(propane-1,3-diyl)bis(2-(6-(1H-imidazol-1-yl)pyridazine-3-carbox-amido)-5-fluorobenzoic acid) (3): To a solution of dimethyl 4,4′-(propane-1,3-diyl)bis(2-(6-(1H-imidazol-1-yl)pyridazine-3-carboxamido)-5-fluorobenzoate) (1.5 g, 2.07 mmol, 1 eq.) in ACN (7.5 mL) and Water (7.5 mL) was added TEA (2.91 ml, 20.76 mmol, 10 eq.) at rt. The reaction mixture was stirred at 115-120° C. for 3 h (under seal tube). After completion of the reaction, the reaction mixture was evaporated under reduced pressure. To the resulted solid, water (20 ml) was added and acidified to 2.0 pH using 1N HCl solution. The resulting precipitate were collected by filtration, washed with water and dried to get brown solid which was further purified by trituration using Methanol (3×10 mL) to get compound 3 (650 mg, 45% yield). ¹H NMR (400 MHz, m DMSO-d₆) δ 9.66 (s, 2H), 8.79 (d, J=9.0 Hz, 2H), 8.60 (d, J=6.3 Hz, 2H), 8.37 (d, J=9.1 Hz, 2H), 8.29 (t, J=1.9 Hz, 2H), 7.90 (d, J=9.6 Hz, 2H), 7.75-7.69 (m, 2H), 2.91 (t, J=7.8 Hz, 4H), 2.14 (d, J=9.5 Hz, 2H). MS-ESI: m/z 695.1 observed [M+H]⁺.

Step 5: Synthesis of magnesium 4,4′-(propane-1,3-diyl)bis(2-(6-(1H-imidazol-1-yl) pyridazine-3-carbox-amido)-5-fluorobenzoate) (3-Mg): 100 mg of compound 3 and 18.57 mg of Mg(OH)₂ (2.1 eqv.) were suspended in 10 mL of 1:1 MeOH-Water. Then the suspension was subjected to a heating-cooling cycle (60° C. to 5° C.) in a Thermomixer for 24 hours.

Thermomixer Conditions:
Step 1: 60° C., 6 hours, 850 rpm Heating rate: 1° C./Minute
Step 2: 5° C., 6 hours, 850 rpm Cooling rate: 0.1° C./Minute
Step 3: 60° C., 6 hours, 850 rpm
Step 4: 5° C., 6 hours, 850 rpm

After reaction, the white solid was collected through centrifugation and dried at RT for 24 hours to give 3-Mg. ¹H NMR (400 MHz, DMSO-d₆) δ 8.75 (d, J=7.2 Hz, 4H), 8.44 (d, J=9.2 Hz, 2H), 8.38 (d, J=9.1 Hz, 2H), 8.16 (t, J=1.5 Hz, 2H), 7.75 (d, J=10.9 Hz, 2H), 7.28-7.19 (m, 2H), 2.75 (t, J=7.7 Hz, 4H), 1.96 (t, J=7.7 Hz, 2H). MS-ESI: m/z 695.44 observed [M+H]+.

Procedures analogous to those for the synthesis of compound 3 were used for the synthesis of compounds such as 13-15, 29, 48, 51-56, 61, 65, 66, 68, 70, 71, 119, 134, 148, 172, 174, 161, 164, 165, 170, 180, 187, 194, 199, 201, 202, 219, 78, 80, 59, 182, and 127.

Step-6: Synthesis of 2-(6-(1H-imidazol-1-yl)pyridazine-3-carboxamido)-4-(3-(5-(6-(1H-imidazol-1-yl)pyridazine-3-carboxamido)-4-(ethoxycarbonyl)-2-fluorophenyl)propyl)-5-fluorobenzoic acid (173): To a solution of compound 3 (0.15 g, 0.216 mmol, 1 eq.) and K₂CO₃ (0.045 g, 0.324 mmol, 1 eq.) in dry DMF (1.5 mL) was added Ethyl iodide (0.034 g, 0.216 mmol, 1 eq.) at rt. The reaction mixture was then stirred at 80° C. for 4 h. After completion of reaction, reaction mixture was diluted with cold water (10 mL). The aqueous layer was extracted with ethyl acetate (3×10 mL) and the combined organic layers were dried over Na₂SO₄ and evaporated to get crude product. The crude material was purified by Prep-HPLC to get pure 173 (1.5 mg) MS-ESI: m/z 723.2 observed [M+H]⁺.

Procedures analogous to those for the synthesis of compound 173 were used for the synthesis of compounds such as 47 and 62. Analogous methodologies also are used to prepare compounds 224-234.

### Example 4

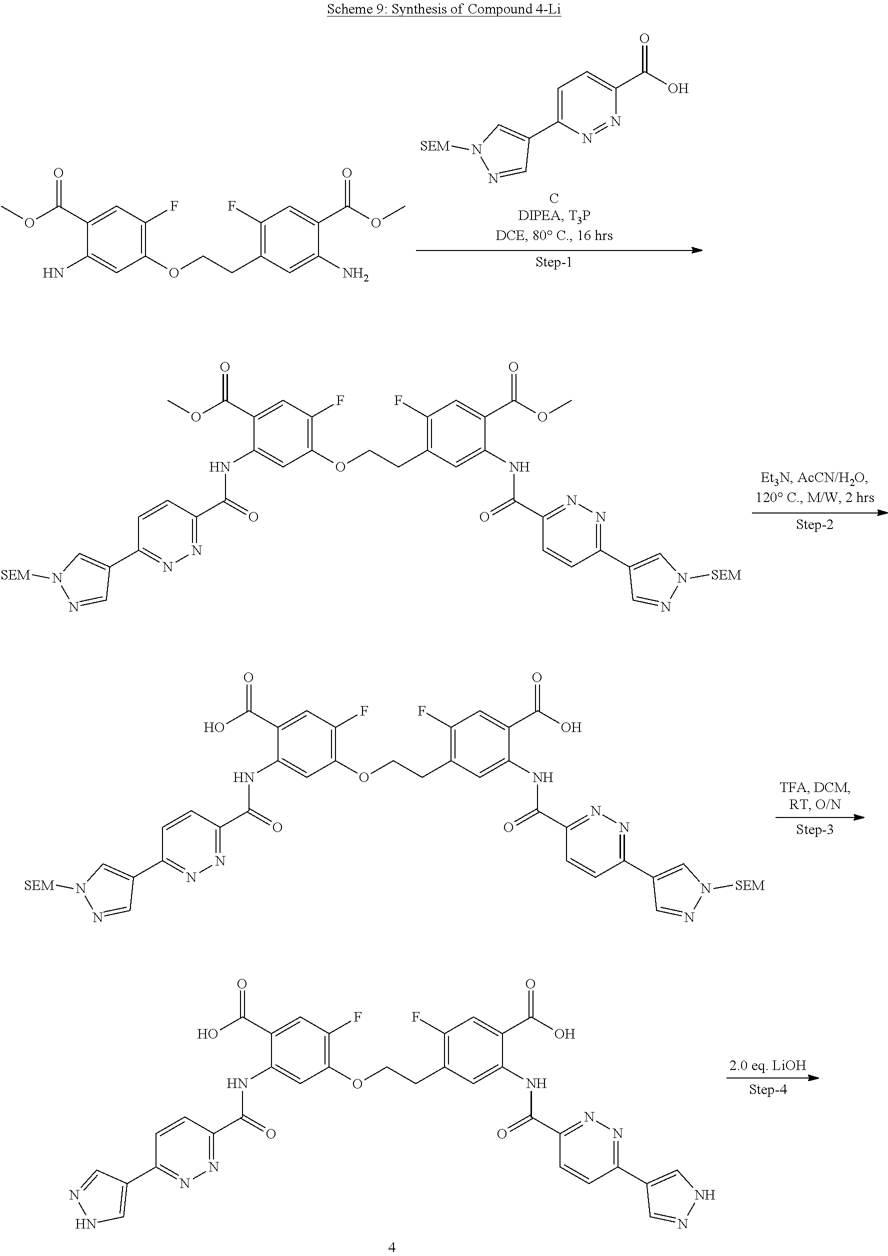

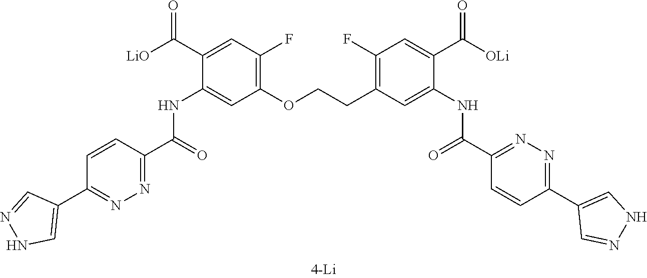

Step 1: Synthesis of methyl 5-fluoro-4-(2-fluoro-4-(methoxycarbonyl)-5-(6-(1-((2-(trimethylsilyl)ethoxy)methyl)-1H-pyrazol-4-yl)pyridazine-3-carboxamido)phenethoxy)-2-(6-(1-((2-(trimethylsilyl)ethoxy)methyl)-1H-pyrazol-4-yl)pyridazine-3-carboxamido) benzoate: To a stirred solution of C (0.32 g, 0.99 mmol, 2.5 eq.) in DCE (7 ml) was added DIPEA (0.46 g, 0.62 ml 3.55 mmol, 9 eq.) and 50% solution of T₃P (in Ethyl Acetate) (1.5 g, 2.37 mmol, 6 eq.) at rt. To this, methyl 2-amino-4-(5-amino-2-fluoro-4-(methoxycarbonyl)phenethoxy)-5-fluoro-benzoate (0.15 g, 0.39 mmol, 1 eq.) was added at room temperature. The reaction mixture was heated at 80-90° C. overnight. After completion of the reaction, the reaction mixture was directly concentrated under vacuum. The crude material was poured in cold water to fall out residue. The crude material was filtered and purified by silica gel column chromatography using 60% Ethyl Acetate in Hexane as eluent to get pure methyl 5-fluoro-4-(2-fluoro-4-(methoxycarbonyl)-5-(6-(1-((2-(trimethylsilyl)ethoxy)methyl)-1H-pyrazol-4-yl)pyridazine-3-carboxamido)phenethoxy)-2-(6-(1-((2-(trimethylsilyl)ethoxy)methyl)-1H-pyrazol-4-yl)pyridazine-3-carboxamido) benzoate (0.23 g, 59.20% yield). MS-ESI: m/z 986.0 observed [M+H]⁺.

Step 2: Synthesis of methyl 5-fluoro-4-(2-fluoro-4-(methoxycarbonyl)-5-(6-(1-((2-(trimethylsilyl)ethoxy)methyl)-1H-pyrazol-4-yl)pyridazine-3-carboxamido)phenethoxy)-2-(6-(1-((2-(trimethylsilyl)ethoxy)methyl)-1H-pyrazol-4-yl)pyridazine-3-carboxamido) benzoate: To a solution of methyl 5-fluoro-4-(2-fluoro-4-(methoxycarbonyl)-5-(6-(1-((2-(trimethylsilyl)ethoxy)methyl)-1H-pyrazol-4-yl)pyridazine-3-carboxamido)phenethoxy)-2-(6-(1-((2-(trimethylsilyl)ethoxy)methyl)-1H-pyrazol-4-yl)pyridazine-3-carboxamido) benzoate (0.150 g, 0.20 mmol, 1 eq.) in ACN (7.5 mL) and Water (7.5 mL) was added TEA (0.2 g, 2.03 mmol, 10 eq.) at rt. The reaction mixture was stirred in microwave irradiation at 120° C. for 4 h. After completion of the reaction, the reaction mixture was distilled out and residue was triturated with Ethyl Acetate to get pure methyl 5-fluoro-4-(2-fluoro-4-(methoxycarbonyl)-5-(6-(1-((2-(trimethylsilyl)ethoxy) methyl)-1H-pyrazol-4-yl)pyridazine-3-carboxamido)phenethoxy)-2-(6-(1-((2-(trimethylsilyl) ethoxy)methyl)-1H-pyrazol-4-yl)pyridazine-3-carboxamido) benzoate (105 mg, 72.05% yield). ¹H NMR (400 MHz, DMSO-d₆) δ 15.17 (s, 2H), 10.1 (s, 2H), 8.88-8.74 (m, 4H), 8.36 (s, 2H), 8.35-8.18 (m, 4H), 7.76-7.73 (t, J=12.8 Hz, 2H), 5.53 (s, 4H), 4.37 (s, 2H), 3.62 (t, J=8.0 Hz, 4H), 3.09 (s, 2H), 0.88 (t, J=8.0 Hz, 4H), 0.0 (s, 18H); MS-ESI: m/z 958.4 observed [M+H]⁺.

Step 3: Synthesis of 2-(6-(1H-pyrazol-4-yl)pyridazine-3-carboxamido)-4-(5-(6-(1H-pyrazol-4-yl)pyridazine-3-carboxamido)-4-carboxy-2-fluorophenethoxy)-5-fluorobenzoic acid (4): To a stirred solution of methyl 5-fluoro-4-(2-fluoro-4-(methoxycarbonyl)-5-(6-(1-((2-(trimethylsilyl)ethoxy)methyl)-1H-pyrazol-4-yl)pyridazine-3-carboxamido)phenethoxy)-2-(6-(1-((2-(trimethylsilyl)ethoxy)methyl)-1H-pyrazol-4-yl)pyridazine-3-carboxamido) benzoate (0.105 g, 0.11 mmol, 1 eq.) in DCM (4 ml) was added TFA (50 mg, 0.44 mmol, 4 eq.) at rt. The reaction mixture was stirred at rt overnight. After completion of the reaction, the reaction mixture was directly concentrated under vacuum. The crude material was triturated with water (5 ml). The residue was purified by prep-HPLC to get compound 4 (26 mg, 34.02% yield). MS-ESI: m/z 697.2 observed [M+H]⁺.

Step 4: Synthesis of lithium 2-(6-(1H-pyrazol-4-yl)pyridazine-3-carboxamido)-4-(5-(6-(1H-pyrazol-4-yl)pyridazine-3-carboxamido)-4-carboxylato-2-fluorophenethoxy)-5-fluoro-benzoate (4): To a suspension of 4 (26 mg, 0.04 mmol, 1 eq.) in water (6 ml) was added LiOH·H₂O (3.3 mg, 0.08 mmol, 2.1 eq.) and the resultant clear solution was filtered to remove any insoluble particles. The solution was lyophilized to obtain compound 4-Li (26 mg). ¹H NMR (500 MHz, DMSO) δ 9.15 (t, J=6.5 Hz, 1H), 8.82 (d, J=7.0 Hz, 1H), 8.70 (dd, J=8.2, 4.2 Hz, 1H), 8.57 (d, J=3.4 Hz, 1H), 8.36-8.05 (m, 6H), 7.73 (d, J=11.6 Hz, 2H), 5.50-5.38 (m, 2H), 4.31 (t, J=7.0 Hz, 2H). MS-ESI: m/z 697.16 observed [M+H]⁺.

Procedures analogous to those for the synthesis of compound 4 were used for the synthesis of compounds 123, 125, 129, 131, 133, 141-144, 150, 152-154, 157, 159, 162, 163, 166, 167, 175, 178, 179, 181, 183, 186, 195, 197, 198, 200, 208, 209, 216, 217, and 238.

### Example 5

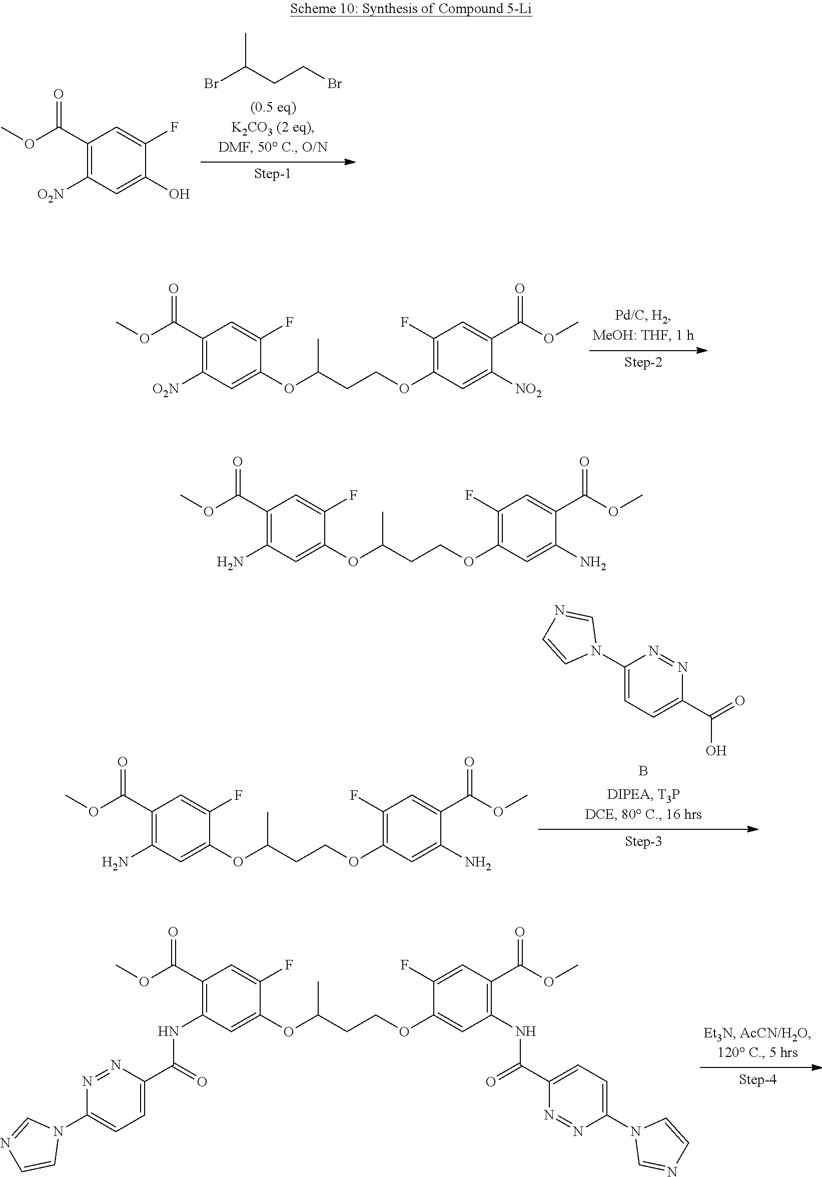

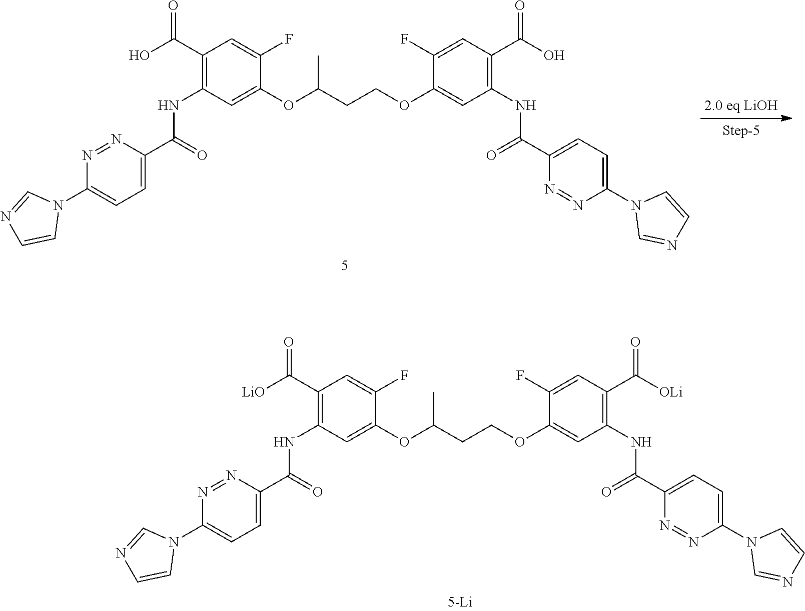

Step 1: Synthesis of dimethyl 4,4′-(butane-1,3-diylbis(oxy))bis(5-fluoro-2-nitrobenzoate): To a solution of Methyl 5-fluoro-4-hydroxy-2-nitrobenzoate (I g, 4.65 mmol, 1 eq.) in DMF (10 mL) was added K₂CO₃ (1.28 g, 9.30 mmol, 2 eq.) and 1,3-dibromobutane (0.5 g, 2.33 mmol, 0.5 eq.) at rt. The resultant solution was stirred at 50° C. for 16 h. After completion of the reaction, reaction mixture was cooled at rt and diluted with water (30 mL). The aqueous layer was extracted with Ethyl Acetate (2×50 mL) and the combined organic layers were dried over anhydrous Na₂SO₄ and evaporated to get crude product. The crude material was purified through silica gel column chromatography using 15% Ethyl Acetate in Hexanes as eluent to afford pure dimethyl 4,4′-(butane-1,3-diylbis(oxy))bis(5-fluoro-2-nitrobenzoate) (0.6 g, 27%) as a solid. ¹HNMR (400 MHz, DMSO-d₆) δ 1.42 (d, J=6.0 Hz, 3H), 2.76 (s, 1H), 2.92 (s, 1H), 3.84 (s, 6H), 4.38 (d, J=4.3 Hz, 2H), 4.97 (d, J=6.1 Hz, 1H), 7.81 (d, J=10.8 Hz, 2H), 7.93 (dd, J=9.4, 7.2 Hz, 2H), MS-ESI: m/z 502 observed [M+18]⁺.

Step 2: Synthesis of dimethyl 4,4′-(butane-1,3-diylbis(oxy))bis(2-amino-5-fluorobenzoate): To a solution of dimethyl 4,4′-(butane-1,3-diylbis(oxy))bis(5-fluoro-2-nitrobenzoate) (0.6 g, 1.23 mmol, 1 eq.) in MeOH (10 mL) and THF (10 mL) was added 10% Pd/C catalyst with 50% moist (0.2 g) at rt. The reaction mixture was purged with Hydrogen gas for 1 h. After completion of the reaction, the reaction mixture was filtered on Celite bed and washed with 10% MeOH in DCM solution. The filtrate was concentrated under vacuum to get crude dimethyl 4,4′-(butane-1,3-diylbis(oxy))bis(2-amino-5-fluorobenzoate) (0.45 g, 86%) which was used in next step without further purification. MS-ESI: m/z 425 observed [M+H]⁺.

Step 3: Synthesis of dimethyl 4,4′-(butane-1,3-diylbis(oxy))bis(2-(6-(1H-imidazol-1-yl)pyridazine-3-carboxamido)-5-fluorobenzoate): To a stirred solution of intermediate B (0.45 g, 2.35 mmol, 2.5 eq.) in DCE (8 ml) was added DIPEA (1.46 g, 2.03 ml, 11.31 mmol, 12 eq.) and 50% solution of T₃P (in Ethyl Acetate) (12.02 mL, 18.86 mmol, 8 eq.) at rt. To this, dimethyl 4,4′-(butane-1,3-diylbis(oxy))bis(2-amino-5-fluorobenzoate) (0.4 g, 0.94 mmol, 1 eq.) was added at rt. The reaction mixture was heated at 80-90° C. overnight. After completion of the reaction, the reaction mixture was then directly concentrated under vacuum. The crude material was purified by silica gel column chromatography using 1.5% to 2% MeOH in DCM as a gradient to afford pure dimethyl 4,4′-(butane-1,3-diylbis(oxy))bis(2-(6-(1H-imidazol-1-yl)pyridazine-3-carboxamido)-5-fluorobenzoate) (0.15 g, 20.7% yield) as a solid. MS-ESI: m/z 769 observed [M+H]⁺.

Step 4: Synthesis of 4,4′-(butane-1,3-diylbis(oxy))bis(2-(6-(1H-imidazol-1-yl)pyridazine-3-carboxamido)-5-fluorobenzoic acid) (5): To a solution of dimethyl 4,4′-(butane-1,3-diylbis(oxy))bis(2-(6-(1H-imidazol-1-yl)pyridazine-3-carboxamido)-5-fluorobenzoate) (150 mg, 0.2 mmol, 1 eq.) in 50% mixture of ACN:Water (15 mL) was added TEA (0.27 mL, 1.95 mmol, 10 eq.) at rt. The reaction mixture was heated in microwave at 120° C. for 4 h. After completion of the reaction, the reaction mixture was directly purified by Prep-HPLC to get pure compound 5 (30 mg, 20.76% yield). MS-ESI: m/z 741.2 observed [M+H]⁺.

Step 5: Synthesis of lithium 4,4′-(butane-1,3-diylbis(oxy))bis(2-(6-(1H-imidazol-1-yl)pyridazine-3-carboxamido)-5-fluorobenzoate) (5-Li): To a suspension of compound 5 (30 mg, 0.04 mmol, 1 eq.) in water (6 mL) was added LiOH·H₂O (3.5 mg, 0.09 mmol, 2.1 eq.) and the resultant clear solution was then filtered to remove any insoluble particles. The resultant solution was lyophilized to obtain 5-Li (27 mg, 90% yield). ¹H NMR (400 MHz, DMSO-d₆) δ 16.08 (s, 1H), 16.05 (s, 1H), 8.78 (s, 2H), 8.73-8.68 (m, 2H), 8.48-8.45 (m, 2H), 8.40 (d, J=8.8 Hz, 2H), 8.19 (s, 2H), 7.75 (dd, J=12.4, 4.4 Hz, 2H), 7.25 (s, 2H), 4.80-4.61 (m, 1H), 4.28-4.26 (m, 2H), 2.34-2.28 (m, 2H), 1.45-1.43 (m, 4H). MS-ESI: m/z 741.2 observed [M+H]⁺.

Procedures analogous to those for the synthesis of compound 5 were used for the synthesis of compounds 11, 12, 16, 17, 21, 23, 34, 36, 37, 38, 42, 43, 45, 50, 138, 139, 168, 185, 206, and 220.

### Example 6

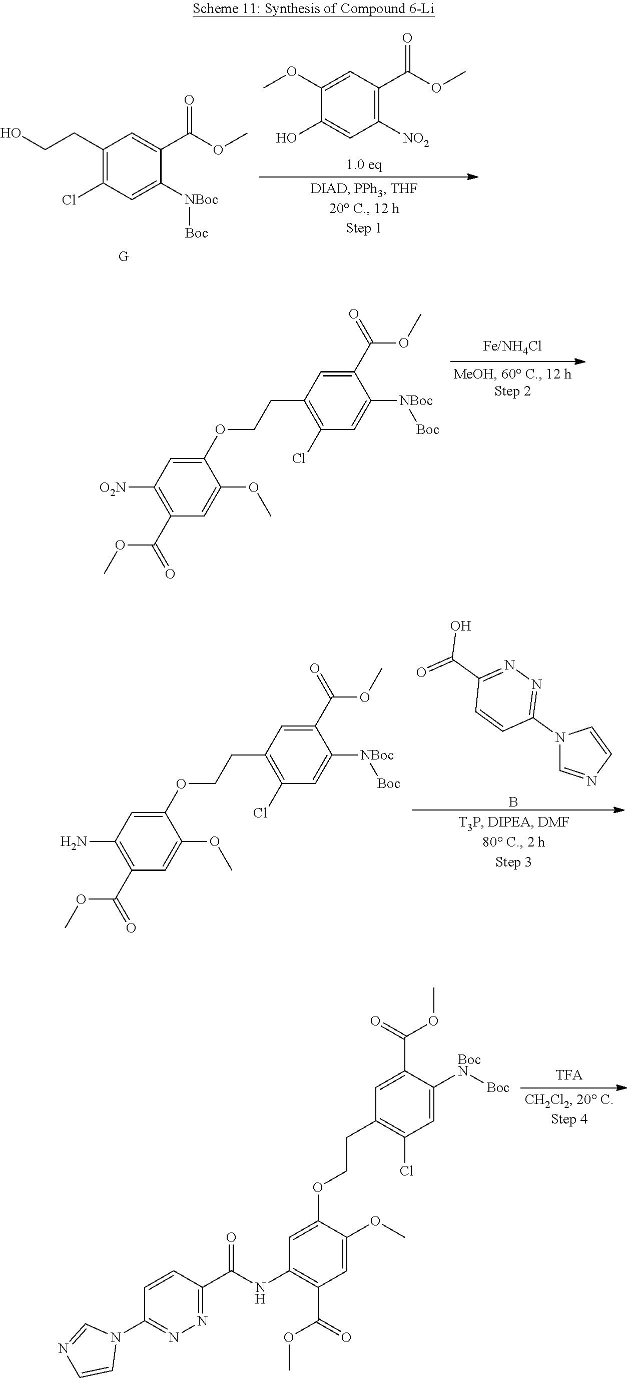

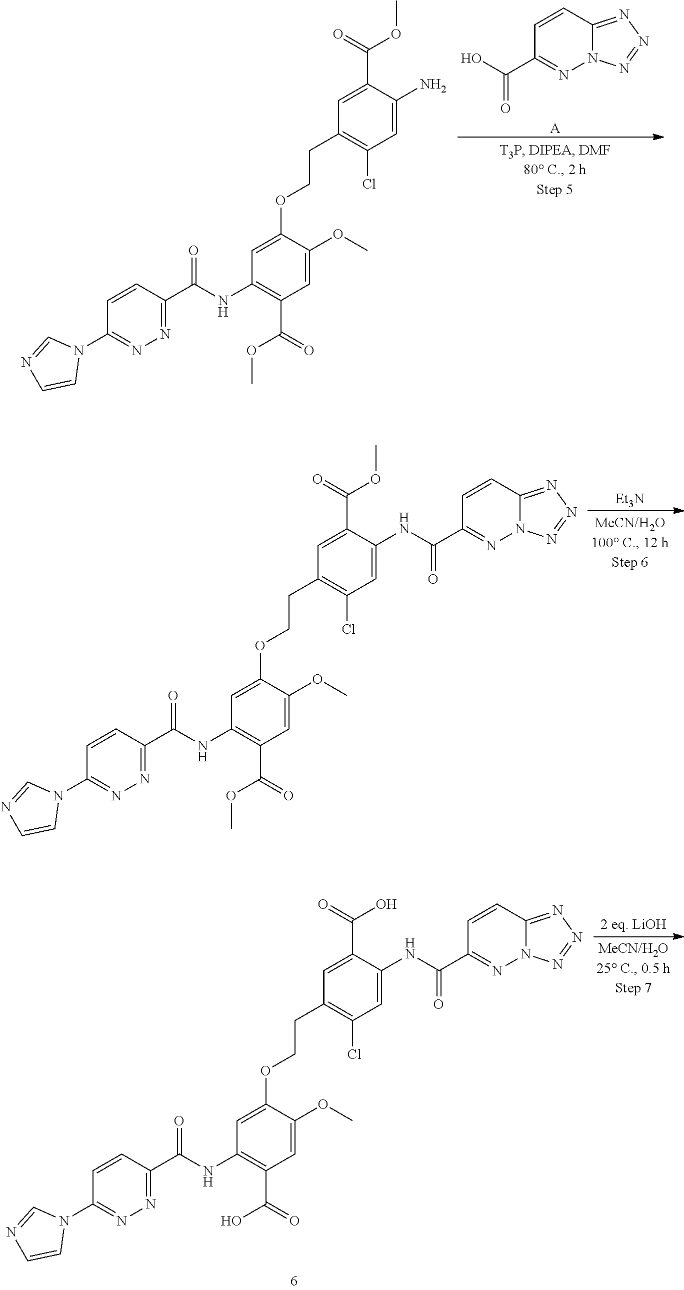

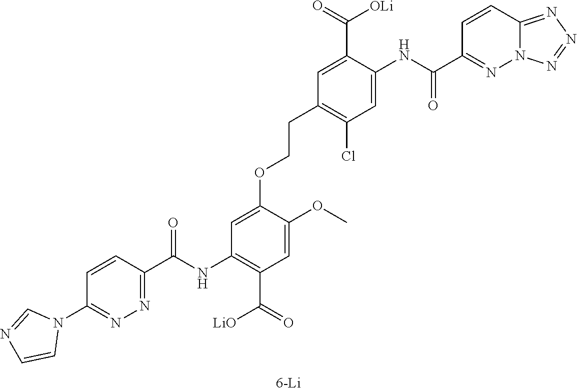

Step 1: Synthesis of methyl 2-(bis(tert-butoxycarbonyl)amino)-4-chloro-5-(2-(2-methoxy-4-(methoxycarbonyl)-5-nitrophenoxy)ethyl)benzoate: To a solution of methyl 2-[bis(tert-butoxycarbonyl)amino]-4-chloro-5-(2-hydroxyethyl)benzoate (500 mg, 1.16 mmol, 1 eq) and methyl 4-hydroxy-5-methoxy-2-nitro-benzoate (264 mg, 1.16 mmol, 1 eq) in THF (10 mL) was added DIAD (352 mg, 1.74 mmol, 0.339 mL, 1.5 eq) and PPh₃ (457 mg, 1.74 mmol, 1.5 eq). The reaction mixture was stirred at 20° C. for 12 hrs. Then the reaction mixture was partitioned between water (20 mL) and Ethyl Acetate (20 mL). The organic phase was separated, washed with brine (20 mL), dried over anhydrous sodium sulfate, filtered and concentrated under reduced pressure to give a crude product. The crude material was purified by flash silica gel chromatography using with 0˜60% Ethyl Acetate/Petroleum Ether as a gradient to afford methyl 2-[bis(tert-butoxycarbonyl)amino]-4-chloro-5-[2-(2-methoxy-4-methoxycarbonyl-5-nitro-phenoxy)ethyl] benzoate (700 mg, 1.05 mmol, 90% yield) as a white solid. MS-ESI: m/z 439.1 observed [M+H]⁺.

Step 2: Synthesis of methyl 2-amino-4-(4-(bis(tert-butoxycarbonyl)amino)-2-chloro-5-(methoxycarbonyl)phenethoxy)-5-methoxybenzoate: To a solution of methyl 2-[bis(tert-butoxycarbonyl)amino]-4-chloro-5-[2-(2-methoxy-4-methoxycarbonyl-5-nitro-phenoxy)ethyl] benzoate (700 mg, 1.10 mmol, 1 eq) in MeOH (10 mL) were added Fe (305 mg, 5.48 mmol, 5 eq) and NH₄Cl (585 mg, 10.95 mmol, 10 eq). The reaction mixture was stirred at 60° C. for 12 hrs. The reaction mixture was filtered, and the filtrate was concentrated under vacuum. The residue was diluted with Ethyl Acetate (15 mL) and extracted with water (15 mL×3). The combined organic layers were washed with brine 20 mL, dried over anhydrous Na₂SO₄, filtered and concentrated under reduced pressure to give methyl 2-amino-4-(4-(bis(tert-butoxycarbonyl)amino)-2-chloro-5-(methoxycarbonyl)phenethoxy)-5-methoxybenzoate (540 mg, crude) as a brown oil. The crude product was used to next step without further purification.

Step 3: Synthesis of methyl 2-(6-(1H-imidazol-1-yl)pyridazine-3-carboxamido)-4-(4-(bis(tert-butoxycarbonyl)amino)-2-chloro-5-(methoxycarbonyl)phenethoxy)-5-methoxy-benzoate: To a solution of Intermediate B (234 mg, 1.23 mmol, 1.5 eq) and methyl 5-[2-(5-amino-2-methoxy-4-methoxycarbonyl-phenoxy)ethyl]-2-[bis(tert-butoxycarbonyl)amino]-4-chloro-benzoate (500 mg, 0.820 mmol, 1 eq) in DMF (10 mL) were added T₃P (4.18 g, 6.57 mmol, 3.91 mL, 8 eq) and DIPEA (1.59 g, 12.31 mmol, 2.14 mL, 15 eq). The mixture was stirred at 80° C. for 12 hrs. Water (15 mL) was added and the resultant mixture was stirred at 25° C. for another 30 min. The crude material was purified by flash silica gel chromatography using 0˜100% ethyl acetate/petroleum ether as a gradient to afford methyl 2-(6-(1H-imidazol-1-yl)pyridazine-3-carboxamido)-4-(4-(bis(tert-butoxycarbonyl)amino)-2-chloro-5-(methoxycarbonyl)phenethoxy)-5-methoxy-benzoate (480 mg, 74% yield) as a brown solid.

Step 4: Synthesis of methyl 2-(6-(1H-imidazol-1-yl)pyridazine-3-carboxamido)-4-(4-amino-2-chloro-5-(methoxycarbonyl)phenethoxy)-5-methoxybenzoate: To a solution of methyl 2-(6-(1H-imidazol-1-yl)pyridazine-3-carboxamido)-4-(4-(bis(tert-butoxycarbonyl)amino)-2-chloro-5-(methoxycarbonyl)phenethoxy)-5-methoxy-benzoate (480 mg, 0.614 mmol, 1 eq) in CH₂Cl₂ (5 mL) was added TFA (7.70 g, 67.5 mmol, 5.00 mL, 109 eq). The mixture was stirred at 20° C. for 2 hrs. The reaction mixture was concentrated under reduced pressure, washed with CH₂Cl₂ (5 mL×3) to give a residue. The crude product was triturated with Ethyl Acetate to afford methyl 2-(6-(1H-imidazol-1-yl)pyridazine-3-carboxamido)-4-(4-amino-2-chloro-5-(methoxy-carbonyl) phenethoxy)-5-methoxybenzoate (210 mg, 53% yield) as a gray solid. MS-ESI: m/z 581.2 observed [M+H]⁺.

Step 5: Synthesis of methyl 2-(6-(1H-imidazol-1-yl)pyridazine-3-carboxamido)-4-(2-chloro-5-(methoxycarbonyl)-4-(tetrazolo[1,5-b]pyridazine-6-carboxamido)phenethoxy)-5-methoxybenzoate: To a solution of intermediate A (89.5 mg, 0.542 mmol, 1.5 eq) and methyl 2-amino-4-chloro-5-[2-[5-[(6-imidazol-1-ylpyridazine-3-carbonyl)amino]-2-methoxy-4-methoxy-carbonyl-phenoxy]ethyl]benzoate (210 mg, 0.361 mmol, 1.0 eq) in DMF (4 mL) were added T₃P (1.84 g, 2.89 mmol, 1.72 mL, 8 eq) and DIPEA (700 mg, 5.42 mmol, 0.944 mL, 15 eq). The reaction mixture was stirred at 80° C. for 12 hrs. Ethyl Acetate (20 mL) was added to the reaction mixture and stirred at 25° C. for 30 min. The mixture was filtered and the filter cake was washed with water (15 mL), Acetonitrile (5 mL×3), Ethyl Acetate (5 mL×3), Petroleum Ether (5 mL×3) and dried under reduced pressure to afford methyl 2-(6-(1H-imidazol-1-yl)pyridazine-3-carboxamido)-4-(2-chloro-5-(methoxycarbonyl)-4-(tetrazolo[1,5-b]pyridazine-6-carboxamido) phenethoxy)-5-methoxy benzoate (180 mg, 66% yield) as a light yellow solid. MS-ESI: m/z 728.1 observed [M+H]⁺.

Step 6: Synthesis of 2-(6-(1H-imidazol-1-yl)pyridazine-3-carboxamido)-4-(5-carboxy-2-chloro-4-(tetrazolo[1,5-b]pyridazine-6-carboxamido)phenethoxy)-5-methoxybenzoic acid (6): To a solution of methyl 2-(6-(1H-imidazol-1-yl)pyridazine-3-carboxamido)-4-(2-chloro-5-(methoxycarbonyl)-4-(tetrazolo[1,5-b]pyridazine-6-carboxamido)phenethoxy)-5-methoxy-benzoate (170 mg, 0.233 mmol, 1 eq) in acetonitrile (5 mL) and water (5 mL) was added Et₃N (3.64 g, 35.9 mmol, 5 mL, 153 eq). The mixture was stirred at 120° C. for 4 hrs. The reaction mixture was concentrated under reduced pressure. The crude material was purified by prep-HPLC to afford compound 6 (20 mg, 10% yield) as a yellow solid. ¹H NMR (400 MHz, DMSO-d₆) δ=8.95 (d, J=9.6 Hz, 1H), 8.82 (s, 1H), 8.78 (s, 1H), 8.63 (s, 1H), 8.50 (d, J=9.2 Hz, 1H), 8.42 (d, J=9.2 Hz, 1H), 8.35 (d, J=9.6 Hz, 1H), 8.19 (s, 1H), 8.16 (s, 1H), 7.62 (s, 1H), 7.26 (s, 1H), 4.26 (t, J=7.6 Hz, 2H), 3.78 (s, 3H), 3.25 (t, J=7.2 Hz, 2H). MS-ESI: m/z 700.2 observed [M+H]⁺

Step 7: Synthesis of lithium 2-(6-(1H-imidazol-1-yl)pyridazine-3-carboxamido)-4-(5-carboxylato-2-chloro-4-(tetrazolo[1,5-b]pyridazine-6-carboxamido)phenethoxy)-5-methoxybenzoate (6-Li): To a solution of compound 6 (20 mg, 0.028 mmol, 1 eq) in water (3 mL) and acetonitrile (3 mL) was added LiOH (0.02 M, 2.86 mL, 2 eq). The mixture was stirred at 20° C. for 0.5 hr. The reaction mixture was lyophilized to give compound 6-Li. ¹H NMR (400 MHz, DMSO-d₆) δ 15.59 (s, 1H), 8.94 (d, J=9.6 Hz, 1H), 8.81 (s, 1H), 8.76 (s, 1H), 8.59 (s, 1H), 8.45 (d, J=9.2 Hz, 1H), 8.38 (d, J=8.8 Hz, 1H), 8.35 (d, J=9.6 Hz, 1H), 8.17 (s, 2H), 7.67 (s, 1H), 7.25 (s, 1H), 4.21 (t, J=7.2 Hz, 2H), 3.76 (s, 3H), 3.23 (t, J=7.2 Hz, 2H). MS-ESI: m/z 700.2 observed [M+H]⁺.

Procedures analogous to those for the synthesis of compound 6 were used for the synthesis of compounds 84, 90, 93, 94, 96, 101, 103, 108, 128, 130, 145, 147, 156, 169, 176, 177, 188-190, 193, 204, 222, and 237.

### Example 7

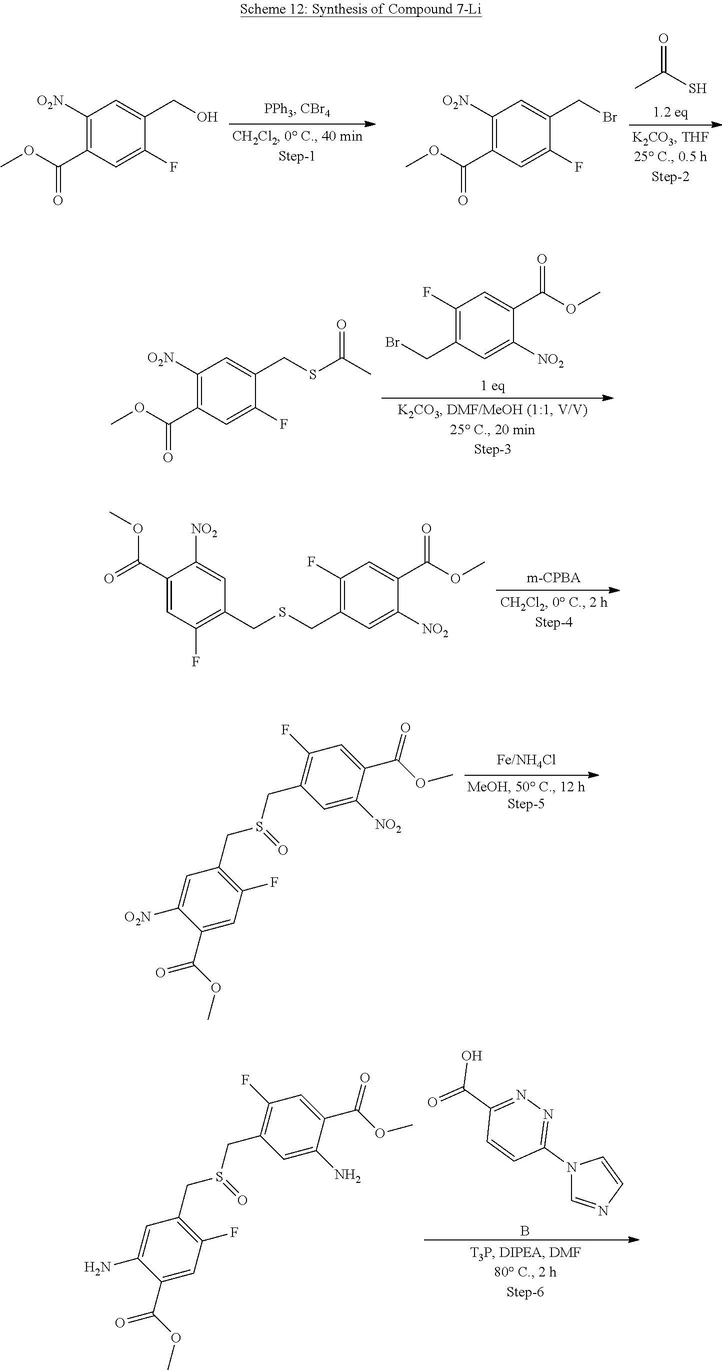

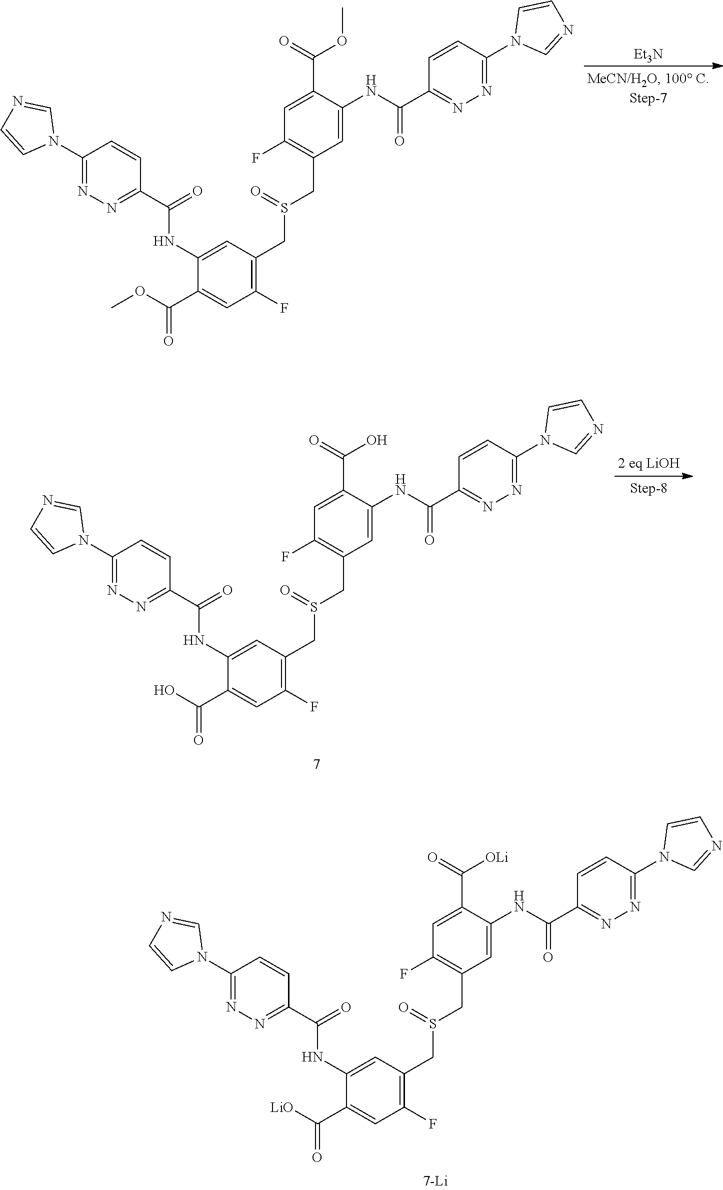

Step 1: Synthesis of methyl 4-(bromomethyl)-5-fluoro-2-nitrobenzoate: To a solution of methyl 5-fluoro-4-(hydroxymethyl)-2-nitro-benzoate (6 g, 26.1 mmol, 1 eq) in DCM (100 mL) was added PPh₃ (13.7 g, 52.3 mmol, 2 eq) at 0° C. and then CBr₄ (17.3 g, 52.3 mmol, 2 eq). The reaction mixture was stirred at 0° C. for 0.5 hour. After completion of the reaction, water (60 mL) was added to the reaction mixture and extracted with DCM (40 mL×3). The combined organic layers were washed with brine (30 mL), dried over anhydrous Na₂SO₄, filtered, and concentrated under reduced pressure to give a crude product. The crude material was purified by flash silica gel chromatography using 0˜20% ethyl acetate/petroleum ether as a gradient to afford methyl 4-(bromomethyl)-5-fluoro-2-nitro-benzoate (6.6 g, 73% yield) as a brown solid. ¹H NMR (400 MHz, DMSO-d₆) δ 8.45 (d, J=6.4 Hz, 1H), 7.85 (d, J=10.4 Hz, 1H), 4.80 (s, 2H), 3.88 (s, 3H).

Step 2: Synthesis of methyl 4-((acetylthio)methyl)-5-fluoro-2-nitrobenzoate: To a solution of methyl 4-(bromomethyl)-5-fluoro-2-nitro-benzoate (3 g, 10.2 mmol, 1 eq) in THF (30 mL) was added K₂CO₃ (2.84 g, 20.5 mmol, 2 eq), and ethanethioic S-acid (938 mg, 12.3 mmol, 0.876 mL, 1.2 eq) slowly, then the reaction mixture was stirred at 20° C. for 0.5 hour. After completion of the reaction, the reaction mixture was added to water (20 mL) and extracted with Ethyl Acetate (30 mL×2), then the combined phase was dried and concentrated under reduced pressure. The crude material was purified by flash silica gel chromatography using 0˜20% ethyl acetate/petroleum ether as a gradient to afford compound methyl 4-(acetylsulfanylmethyl)-5-fluoro-2-nitro-benzoate (2.2 g, 71% yield) as a yellow oil. ¹H NMR (400 MHz, CDCl₃) δ 8.05 (d, J=6.0 Hz, 1H), 7.40 (d, J=8.8 Hz, 1H), 4.18 (d, J=0.8 Hz, 2H), 3.94 (s, 3H), 2.40 (s, 3H).

Step 3: Synthesis of dimethyl 4,4′-(thiobis(methylene))bis(5-fluoro-2-nitrobenzoate): To a solution of methyl 4-(acetylsulfanylmethyl)-5-fluoro-2-nitro-benzoate (2.17 g, 7.57 mmol, 1.3 eq) and methyl 4-(bromomethyl)-5-fluoro-2-nitro-benzoate (1.7 g, 5.82 mmol, 1 eq) in DMF (8 mL) and MeOH (8 mL) was added K₂CO₃ (402 mg, 2.91 mmol, 0.5 eq). The reaction mixture was stirred at 25° C. for 20 min. After completion of the reaction, water (20 mL) was added to the reaction mixture and then the mixture was extracted with Ethyl Acetate (30 mL×3). The combined organic phase was washed with brine (20 mL), dried over anhydrous Na₂SO₄, filtered and concentrated under reduced pressure. The crude material was purified by flash silica gel chromatography using 0˜20% Ethyl acetate/Petroleum ether as a gradient to afford dimethyl 4,4′-(thiobis(methylene))bis(5-fluoro-2-nitrobenzoate) (910 mg, 33% yield) as a yellow solid. ¹H NMR (400 MHz, CDCl₃) δ 8.00 (d, J=6.0 Hz, 2H), 7.40 (d, J=8.8 Hz, 2H), 3.96 (s, 6H), 3.79 (s, 4H). MS-ESI: m/z 474.0 observed [M+H]⁺.

Step 4: Synthesis of dimethyl 4,4′-(sulfinylbis(methylene))bis(5-fluoro-2-nitrobenzoate): To a mixture of dimethyl 4,4′-(thiobis(methylene))bis(5-fluoro-2-nitrobenzoate) (150 mg, 0.329 mmol, 1 eq) in DCM (10 mL) was added m-CPBA (66.7 mg, 0.329 mmol, 1 eq) at 0° C. and then the reaction mixture was stirred at 0° C. for 2 h. After completion of the reaction, the reaction mixture was quenched with aqueous NaHCO₃ (20 mL) and extracted with DCM (10 mL×3). The combined organic layers were dried, filtered and concentrated under reduced pressure to afford dimethyl 4,4′-(sulfinylbis(methylene))bis(5-fluoro-2-nitrobenzoate) (210 mg, crude) as a white solid. The crude product was used directly for the next step without further purification. MS-ESI: m/z 473.0 observed [M+H]⁺.

Step 5: Synthesis of dimethyl 4,4′-(sulfinylbis(methylene))bis(2-amino-5-fluorobenzoate): To a mixture of methyl 5-fluoro-4-[(2-fluoro-4-methoxycarbonyl-5-nitro-phenyl)methyl sulfinylmethyl]-2-nitro-benzoate (210 mg, 0.276 mmol, 62% purity, 1 eq) in MeOH (10 mL) were added Fe (77.0 mg, 1.38 mmol, 5 eq) and NH₄Cl (147 mg, 2.76 mmol, 10 eq), the mixture was stirred at 50° C. for 5 h. The reaction mixture was filtered and concentrated under reduced pressure. The crude material was purified by prep-TLC (SiO₂, Petroleum Ether/Ethyl Acetate=1/1) to afford dimethyl 4,4′-(sulfinylbis(methylene))bis(2-amino-5-fluorobenzoate) (30.0 mg, 26% yield) as a white solid. MS-ESI: m/z 413.3 observed [M+H]⁺.

Step 6: Synthesis of dimethyl 4,4′-(sulfinylbis(methylene))bis(2-(6-(1H-imidazol-1-yl)pyridazine-3-carboxamido)-5-fluorobenzoate): To a mixture of dimethyl 4,4′-(sulfinylbis(methylene))bis(2-amino-5-fluorobenzoate) (20.0 mg, 0.048 mmol, 1 eq) and intermediate B (36.9 mg, 0.194 mmol, 4 eq) in DMF (I mL) were added T₃P (123 mg, 0.194 mmol, 0.115 mL, 50% purity, 4 eq) and DIPEA (37.6 mg, 0.291 mmol, 0.051 mL, 6 eq). The mixture was stirred at 80° C. for 12 hours. After completion of the reaction, the reaction mixture was diluted with Ethyl Acetate (4 mL) and filtered. Then the filter cake was added to saturated Na₂CO₃ (5 mL) and stirred at 20° C. for 10 min. The mixture was filtered and filter cake was washed with Ethyl Acetate (1 mL), Acetonitrile (1 mL), PE (1 mL) to afford dimethyl 4,4′-(sulfinylbis(methylene))bis(2-(6-(1H-imidazol-1-yl)pyridazine-3-carboxamido)-5-fluorobenzoate) (18.0 mg, crude) as a white solid. The crude product was used for the next step without further purification. ¹H NMR (400 MHz, DMSO-d₆) δ 12.98 (s, 2H), 10.28 (s, 2H), 8.94 (d, J=6.8 Hz, 2H), 8.78-8.60 (m, 6H), 7.97 (s, 2H), 7.81 (d, J=10.0 Hz, 2H), 4.55 (d, J=12.8 Hz, 2H), 4.32 (d, J=12.8 Hz, 2H), 3.90 (s, 6H). MS-ESI: m/z 757.2 observed [M+H]⁺.

Step 7: Synthesis of 4,4′-(sulfinylbis(methylene))bis(2-(6-(1H-imidazol-1-yl)pyridazine-3-carboxamido)-5-fluorobenzoic acid) (7): To a mixture of dimethyl 4,4′-(sulfinylbis(methylene))bis(2-(6-(1H-imidazol-1-yl)pyridazine-3-carboxamido)-5-fluorobenzoate) (10.0 mg, 0.013 mmol, 1 eq) in ACN (0.5 mL) and H₂O (0.5 mL) was added Et₃N (13.4 mg, 0.132 mmol, 0.018 mL, 10 eq) and the reaction mixture was stirred at 120° C. for 1 hour. Then the reaction mixture was concentrated under reduced pressure to obtain a crude product. The crude material was purified by prep-HPLC to afford compound 7 (8.00 mg, 83% yield) as a white solid. MS-ESI: m/z 729.2 observed [M+H]⁺.

Step 8: Synthesis of lithium 4,4′-(sulfinylbis(methylene))bis(2-(6-(1H-imidazol-1-yl)pyridazine-3-carboxamido)-5-fluorobenzoate) (7-Li): To a suspension of compound 7 (8.00 mg, 0.011 mmol, 1 eq) in H₂O (1 mL) was added LiOH·H₂O (0.02 M, 1.10 mL, 2 eq) and the reaction mixture was stirred at 20° C. for 0.5 hour. Then the reaction mixture was lyophilized to obtain compound 7-Li (8.00 mg, 0.011 mmol) as a white solid. ¹H NMR (400 MHz, DMSO-d₆) δ 15.72 (s, 2H), 8.87-8.82 (m, 2H), 8.77 (s, 2H), 8.48-8.36 (m, 4H), 8.19 (s, 2H), 7.78 (d, J=12.8 Hz, 2H), 7.25 (s, 2H), 4.38 (d, J=13.2 Hz, 2H), 4.18 (s, 2H). LCMS [EST, M+1]: 729.2.

Procedures analogous to those for the synthesis of compound 7 were used for the synthesis of compounds 124, 132, 143, 149, 151, and 155.

### Example 8

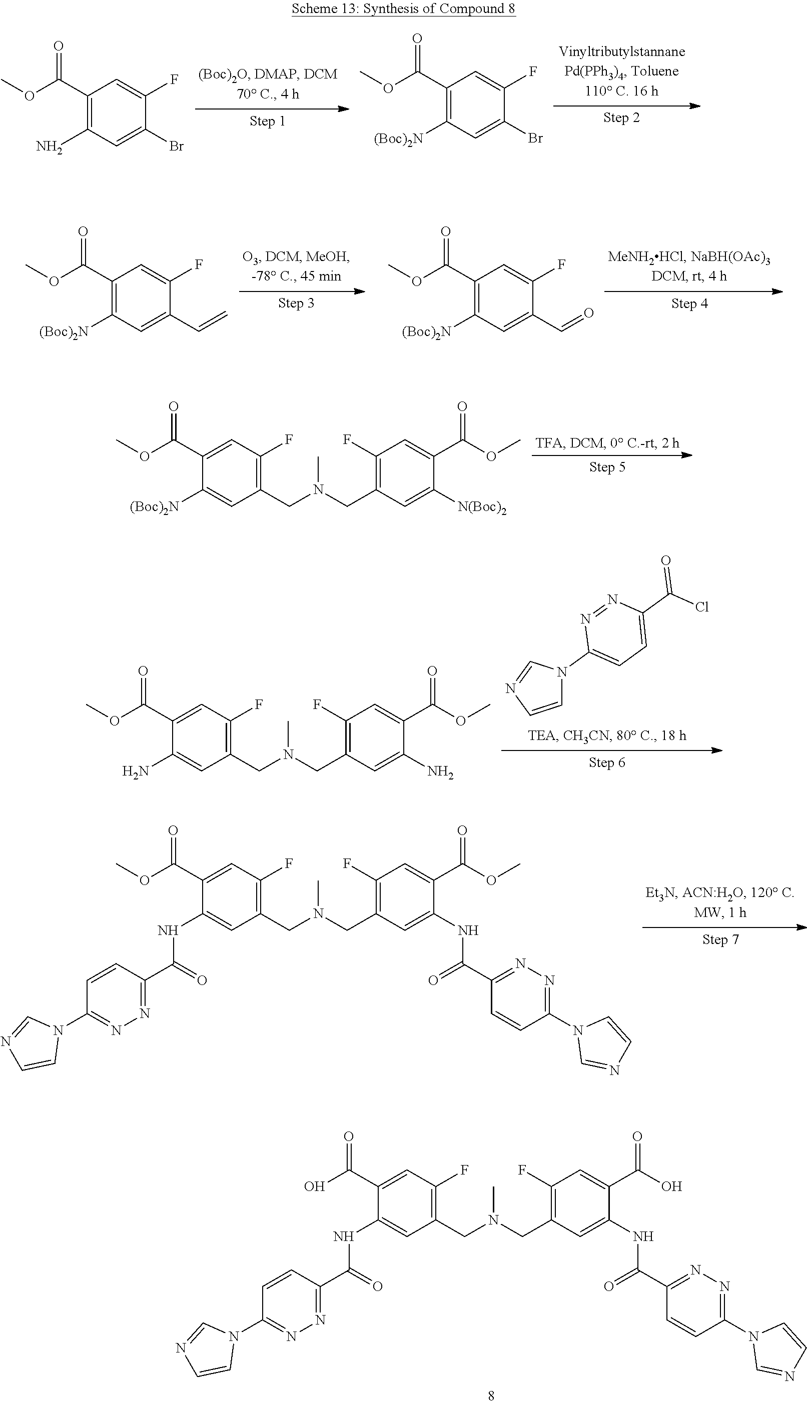

Step 1: Synthesis of methyl 2-(bis(tert-butoxycarbonyl)amino)-4-bromo-5-fluorobenzoate: To a stirred solution of methyl 2-amino-4-bromo-5-fluorobenzoate (1.0 g, 4.03 mmol, 1 eq.) in THF (10 mL) at 0° C., was added di-tert-butyl dicarbonate (1.11 mL, 4.84 mmol, 1.2 eq.) and DMAP (12 mg, 0.40 mmol, 0.1 eq.), the reaction mixture stirred at 70° C. for 4 h. After completion of the reaction, the solvent was removed under reduced pressure then diluted with water (100 mL) and extracted with Ethyl Acetate (3×300 mL). The combined organic layer was dried over anhydrous Na₂SO₄ and concentrated under reduced pressure to give crude product. The crude material was then purified by flash chromatography using 2-3% Ethyl Acetate in petroleum ether as a gradient to afford methyl 2-(bis(tert-butoxycarbonyl)amino)-4-bromo-5-fluorobenzoate (1.4 g, 74% yield) as an off-white solid. MS-ESI: m/z 470.54 observed [M+Na]⁺.

Step 2: Synthesis of methyl 2-(bis(tert-butoxycarbonyl)amino)-5-fluoro-4-vinylbenzoate: To a stirred solution of methyl 2-(bis(tert-butoxycarbonyl)amino)-4-bromo-5-fluorobenzoate (8.5 g, 18.96 mmol, 1 eq.) in toluene (85 mL) at room temperature was added vinyltributylstannane (6.61 g, 20.86 mmol, 1.1 eq.), the resultant mixture was deoxygenated by purging argon gas for 15 min then Pd(PPh₃)₄ (0.44 g, 0.38 mmol, 0.02 eq.) was added and the mixture was stirred at 110° C. for 16 h. After completion of the reaction, the reaction mixture was concentrated under reduced pressure, diluted with water (100 mL) and extracted with Ethyl Acetate (3×100 mL). The combined organic layer was dried over anhydrous Na₂SO₄ and concentrated under reduced pressure to give crude product. The crude residue was then purified by flash chromatography using 2-3% EtOAc in Petroleum Ether as a gradient to afford methyl 2-(bis(tert-butoxycarbonyl)amino)-5-fluoro-4-vinylbenzoate (5.6 g, 75% yield) as a pale yellow solid. MS-ESI: m/z 418.21 observed [M+Na]⁺.

Step 3: Synthesis of methyl 2-(bis(tert-butoxycarbonyl)amino)-5-fluoro-4-formylbenzoate: To a stirred solution of methyl 2-(bis(tert-butoxycarbonyl)amino)-5-fluoro-4-vinylbenzoate (5.6 g, 14.16 mmol, 1 eq.) in MeOH (14 mL) and DCM (42 mL), was purged ozone gas for 45 min at rt. After completion of the reaction, the reaction mixture was concentrated under reduced pressure to afford methyl 2-(bis(tert-butoxycarbonyl)amino)-5-fluoro-4-formylbenzoate (4.7 g, 89% yield) as an off-white solid. MS-ESI: m/z 420.18 observed [M+Na]⁺.

Step 4: synthesis of methyl 4-(((4-((11-oxidaneyl)carbonyl)-5-(bis(tert-butoxycarbonyl)amino)-2-fluorobenzyl)(methyl)amino)methyl)-2-(bis(tert-butoxycarbonyl)amino)-5-fluoro-benzoate: To a stirred solution of methyl 2-(bis(tert-butoxycarbonyl)amino)-5-fluoro-4-formylbenzoate (2.0 g, 5.03 mmol, 2 eq.) in DCM (20 mL) was added methylamine hydrochloride (0.17 g, 2.52 mmol, 1 eq.) followed by STAB (2.13 g, 10.07 mmol, 4.0 eq.) at 0° C. and the reaction mixture was stirred at room temperature for 16 h. After completion of the reaction, the reaction mixture was diluted with water (50 mL) and extracted with DCM (3×70 mL). The combined organic layer was dried over anhydrous Na₂SO₄ and evaporated under reduced pressure to give crude product. The crude residue was then purified by flash chromatography using 25-30% EtOAc in petroleum ether as a gradient to afford methyl 4-(((4-((11-oxidaneyl)carbonyl)-5-(bis(tert-butoxycarbonyl)amino)-2-fluorobenzyl)(methyl)amino)methyl)-2-(bis(tert-butoxycarbonyl)amino)-5-fluorobenzoate (0.65 g, 33% yield) as a colorless gum. MS-ESI: m/z 794.65 observed [M+H]⁺.

Step 5: Synthesis of dimethyl 4,4′-((methylazanediyl)bis(methylene))bis(2-amino-5-fluorobenzoate): To a stirred solution of methyl 4-(((4-((11-oxidaneyl)carbonyl)-5-(bis(tert-butoxycarbonyl)amino)-2-fluorobenzyl)(methyl)amino)methyl)-2-(bis(tert-butoxycarbonyl)amino)-5-fluorobenzoate (0.65 g, 0.82 mmol, 1 eq.) in DCM (3 mL) at 0° C. was added TFA (3 mL) and the reaction mixture stirred at room temperature for 2 h. After completion of the reaction, the reaction mixture was concentrated under reduced pressure to give crude product. The crude residue was then purified by flash chromatography using 25-30% EtOAc in petroleum ether as a gradient to afford dimethyl 4,4′-((methylazanediyl)bis(methylene))bis(2-amino-5-fluorobenzoate) (0.3 g, 96% yield) as a pale brown gum. MS-ESI: m/z 380.08 observed [M+H]⁺.

Step 6: Synthesis of dimethyl 4,4′-((methylazanediyl)bis(methylene))bis(2-(6-(1H-imidazol-1-yl)pyridazine-3-carboxamido)-5-fluorobenzoate): To a stirred solution of dimethyl 4,4′-((methylazanediyl)bis(methylene))bis(2-amino-5-fluorobenzoate) (0.3 g, 0.76 mmol, 1.0 eq.) and DIPEA (1.06 mL, 6.10 mmol, 8.0 eq.) in ACN (3 mL) at room temperature was added 6-(1H-imidazol-1-yl)pyridazine-3-carbonyl chloride (0.48 g, 2.29 mmol, 3.0 eq.) and the mixture was stirred at 80° C. for 2 h. After completion of the reaction, the reaction mixture was diluted with water (50 mL) and the precipitate was filtered, dried under vacuum. The crude was then purified by flash chromatography using 2-5% MeOH in DCM as a gradient to afford dimethyl 4,4′-((methylazanediyl)bis(methylene))bis(2-(6-(1H-imidazol-1-yl)pyridazine-3-carboxamido)-5-fluorobenzoate) (115 mg, 12% yield) as an off-white solid. MS-ESI: m/z 738.70 observed [M+H]⁺.

Step 7: Synthesis of 4,4′-((methylazanediyl)bis(methylene))bis(2-(6-(1H-imidazol-1-yl)pyridazine-3-carboxamido)-5-fluorobenzoic acid) (8): To a stirred solution of dimethyl 4,4′-((methylazanediyl)bis(methylene))bis(2-(6-(1H-imidazol-1-yl)pyridazine-3-carbox-amido)-5-fluorobenzoate) (100 mg, 0.14 mmol, 1.0 eq.) in ACN (1 mL) and H₂O (1 mL) was added Et₃N (0.38 mL, 2.71 mmol, 20 eq.) and the mixture was heated at 120° C. for 1 h using microwave reactor. After completion of the reaction, the reaction mixture was concentrated under reduced pressure, the crude residue was then purified by prep-HPLC to afford compound 8 (40 mg, 40% yield) as an off-white solid. ¹H NMR (400 MHz, DMSO-d₆) δ 15.70 (s, 2H), 8.85 (d, J=7.0 Hz, 2H), 8.77 (s, 2H), 8.45-8.30 (m, 4H), 8.18 (s, 2H), 7.71 (d, J=10.8 Hz, 2H), 7.25 (s, 2H), 3.66 (s, 4H), 2.20 (s, 3H). MS-ESI: m/z 710.47 observed [M+H]⁺.

Procedures analogous to those for the synthesis of compound 8 were used for the synthesis of compounds 235 and 236.

### Example 9

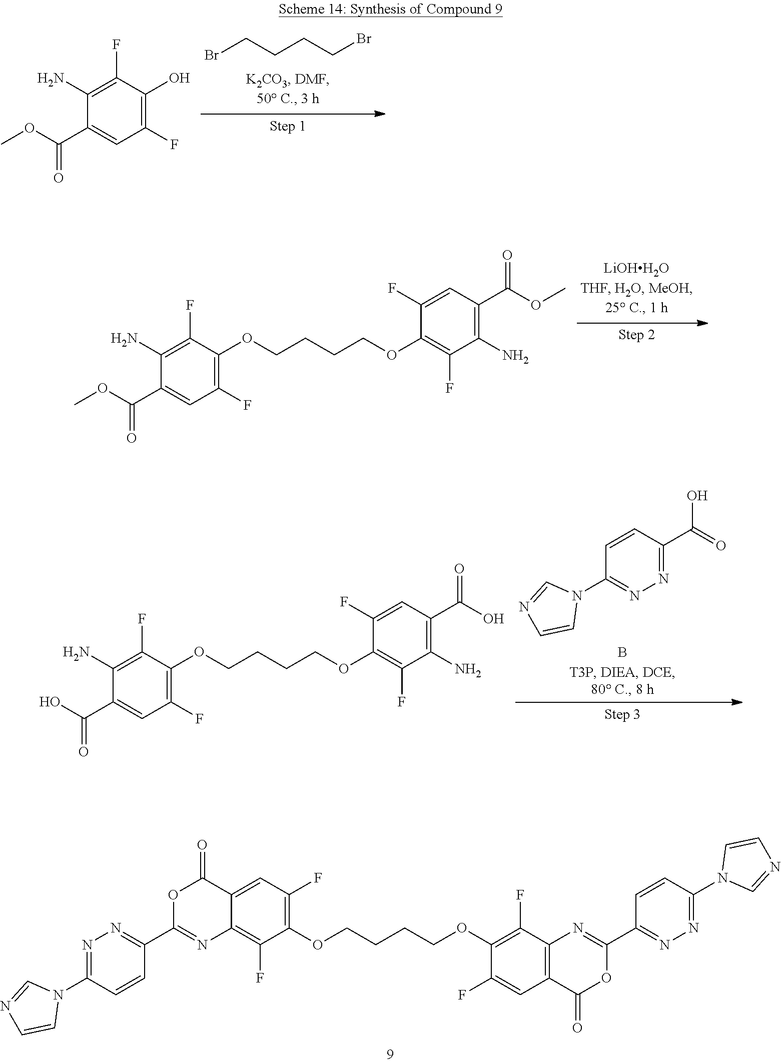

Step 1: Synthesis of methyl 2-amino-4-[4-(3-amino-2,6-difluoro-4-methoxycarbonyl-phenoxy)butoxy]-3,5-difluoro-benzoate: To a solution of methyl 2-amino-3,5-difluoro-4-hydroxy-benzoate (800 mg, 3.94 mmol, 2.00 eq.) and 1,4-dibromobutane (425 mg, 1.97 mmol, 238 uL, 1.00 eq.) in DMF (12.0 mL) was added K₂CO₃ (1.63 g, 11.8 mmol, 6.00 eq.). After stirred at 50° C. for 3 hours, the reaction mixture was diluted with Ethyl Acetate (80.0 mL), washed with water (80 mL×3), dried over anhydrous Na₂SO₄ and filtered. The filtrate was concentrated under reduced pressure to give a crude product. The crude material was purified by silica gel column chromatography to give methyl 2-amino-4-[4-(3-amino-2,6-difluoro-4-methoxycarbonyl-phenoxy)butoxy]-3,5-difluoro-benzoate (756 mg, 83% yield) as a white solid. ¹H NMR (400 MHz, DMSO-d₆) δ 7.36 (dd, J=2.0, 12.4 Hz, 2H), 6.47 (s, 4H), 4.38-4.17 (m, 4H), 3.80 (s, 6H), 1.88-1.81 (m, 4H). LCMS (ESI): m/z 461.1 [M+H]⁺.

Step 2: Synthesis of 2-amino-4-[4-(3-amino-4-carboxy-2,6-difluoro-phenoxy)butoxy]-3,5-difluoro-benzoic acid: To a solution of methyl 2-amino-4-[4-(3-amino-2,6-difluoro-4-methoxycarbonyl-phenoxy)butoxy]-3,5-difluoro-benzoate (300 mg, 0.652 mmol, 1.00 eq.) in THF (1.50 mL), H₂O (1.50 mL) and MeOH (1.50 mL) was added LiOH·H₂O (274 mg, 6.52 mmol, 10.0 eq). The mixture was stirred at 25° C. for 1 hour. The reaction mixture was quenched with a solution of HCl (0.1 N) at 0° C. to pH=7. Then the precipitate was filtered to get a white solid. The crude product was triturated with ACN at 25° C. to give 2-amino-4-[4-(3-amino-4-carboxy-2,6-difluoro-phenoxy)butoxy]-3,5-difluoro-benzoic acid (275 mg, crude) as a white solid. ¹H NMR (400 MHz, DMSO-d₆) δ=7.37 (dd, J=2.0, 12.4 Hz, 2H), 6.56 (br s, 4H), 4.12 (br s, 4H), 1.83 (br s, 4H). LCMS (ESI): m/z 433.1 [M+H]⁺.

Step 3: Synthesis of 7,7′-(butane-1,4-diylbis(oxy))bis(2-(6-(1H-imidazol-1-yl)pyridazin-3-yl)-6,8-difluoro-4H-benzo[d][1,3]oxazin-4-one) (3): To a solution of 2-amino-4-[4-(3-amino-4-carboxy-2,6-difluoro-phenoxy)butoxy]-3,5-difluoro-benzoic acid (140 mg, 0.324 mmol, 1.00 eq.) and compound B (308 mg, 1.62 mmol, 5.00 eq.) in DCE (8.00 mL) was added DIPEA (419 mg, 3.24 mmol, 0.564 mL, 10.0 eq.) and T₃P (1.24 g, 1.94 mmol, 1.16 mL, 50% purity in ethyl acetate, 6.00 eq.). The mixture was stirred at 80° C. for 8 hours. The reaction mixture was concentrated under reduced pressure to give a residue. The residue was washed with saturated NaHCO₃ (5 mL) and water (4 mL) to get a gray solid. The crude product was triturated with ACN at 25° C. for 5 min., then filtered and the filter cake was dried under vacuum to give compound 9 (73.6 mg, two steps 31% yield) as a yellow solid. ¹H NMR (400 MHz, DMSO-d₆) δ 8.79 (s, 2H), 8.64 (d, J=9.2 Hz, 2H), 8.42 (d, J=9.2 Hz, 2H), 8.19 (s, 2H), 7.98 (dd, J=1.2, 10.4 Hz, 2H), 7.26 (s, 2H), 4.52 (br s, 4H), 1.99 (br s, 4H). MS-ESI: m/z 741.3 observed [M+H]⁺.

Procedures analogous to those for the synthesis of compound 9 were used for the synthesis of compounds 40, 41, and 46.

### Example 10

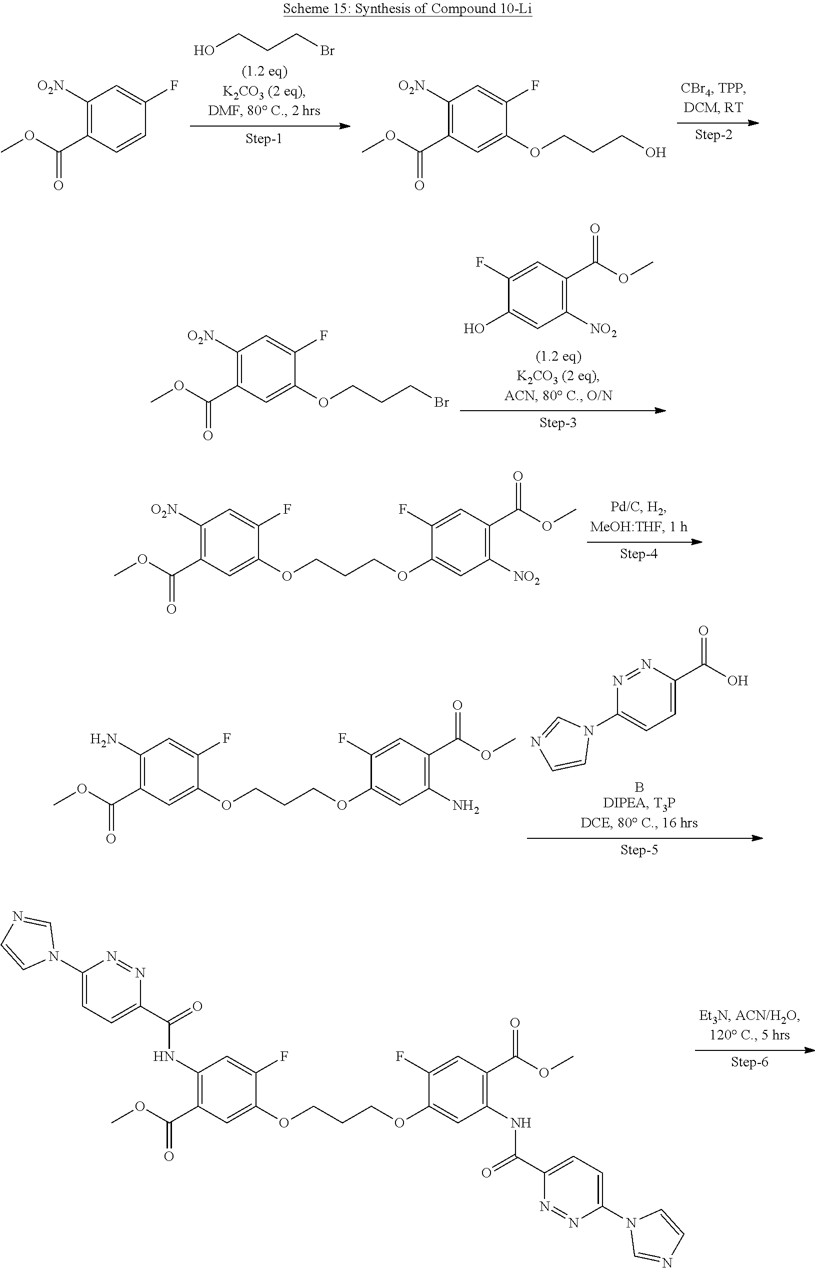

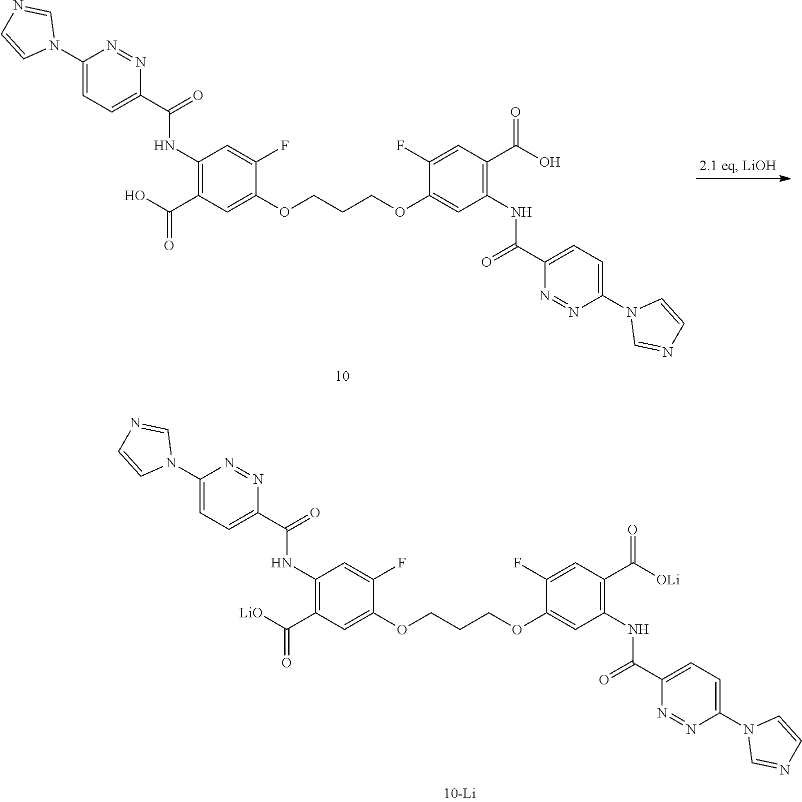

Step 1: Synthesis of methyl 4-fluoro-5-(3-hydroxypropoxy)-2-nitrobenzoate: To a solution of Methyl 4-fluoro-5-hydroxy-2-nitrobenzoate (2 g, 9.30 mmol, 1 eq.) in DMF (20 mL) was added K₂CO₃ (2.56 g, 1.86 mmol, 2 eq.) and 3-bromopropan-1-ol (1.55 g, 1.12 mmol, 1.2 eq.) at rt. The resultant solution was stirred at 80° C. for 2 h. After completion of the reaction, reaction mixture was cooled at rt and diluted with water (50 mL). The aqueous layer was extracted with Ethyl Acetate (2×100 mL) and the combined organic layers were dried over anhydrous Na₂SO₄ and evaporated under reduced pressure to get a crude product. The crude material was purified through silica gel column chromatography using 30% Ethyl Acetate in Hexanes as eluent to get pure methyl 4-fluoro-5-(3-hydroxypropoxy)-2-nitrobenzoate (1.8 g, 71% yield) as a solid. ¹H NMR (400 MHz, DMSO-d₆) δ 8.19 (d, J=10.8 Hz, 1H), 7.62 (d, J=8.0 Hz, 1H), 4.65 (t, J=5.2 Hz, 1H), 4.32 (t, J=6.3 Hz, 2H), 3.87 (s, 3H), 3.59 (d, J=5.9 Hz, 2H), 1.93 (p, J=6.3 Hz, 2H). MS-ESI: m/z 273.0 observed [M+H]⁺.

Step 2: methyl 5-(3-bromopropoxy)-4-fluoro-2-nitrobenzoate: To a solution of methyl 4-fluoro-5-(3-hydroxypropoxy)-2-nitrobenzoate (1.80 g, 6.59 mmol, 1 eq.) in DCM (18 mL) was added CBr₄ (1.10 g, 9.89 mmol, 1.5 eq.) and PPh₃ (2.59 g, 9.89 mmol, 1.5 eq.) at rt. The resultant solution was stirred at rt for 2 h. After completion of the reaction, reaction mixture was diluted with water (50 mL). The aqueous layer was extracted with Ethyl acetate (2×100 mL) and the combined organic layers were dried over anhydrous Na₂SO₄ and evaporated to get crude product. The crude material was purified through silica gel column chromatography using 5% Ethyl Acetate in Hexanes as eluent to get pure methyl 5-(3-bromopropoxy)-4-fluoro-2-nitrobenzoate (1 g, 45% yield) as a solid. ¹H NMR (400 MHz, DMSO-d₆) δ 8.22 (dd, J=10.8, 3.5 Hz, 1H), 7.67 (d, J=8.0 Hz, 1H), 4.37 (t, J=5.9 Hz, 2H), 3.87 (s, 3H), 3.67 (t, J=6.5 Hz, 2H), 2.33 (s, J=5.9 Hz, 2H). MS-ESI: m/z 336.0 observed [M+H]⁺.

Step 3: synthesis of methyl 4-fluoro-5-(3-(2-fluoro-4-(methoxycarbonyl)-5-nitrophenoxy) propoxy)-2-nitrobenzoate: To a solution of Methyl 5-fluoro-4-hydroxy-2-nitrobenzoate (0.384 g, 1.78 mmol, 1.2 eq.) in ACN (5 mL) was added K₂CO₃ (1.28 g, 2.97 mmol, 2 eq.) and methyl 5-(3-bromopropoxy)-4-fluoro-2-nitrobenzoate (0.5 g, 1.48 mmol, 1 eq.) at rt. The resultant solution was stirred at 80° C. for 16 h. After completion of the reaction, reaction mixture was cooled at rt and diluted with water (25 mL). The aqueous layer was extracted with Ethyl Acetate (2×30 mL) and the combined organic layers were dried over anhydrous Na₂SO₄ and evaporated to get crude product. The crude material was purified through silica gel column chromatography using 15% Ethyl Acetate in Hexanes as eluent to get pure methyl 4-fluoro-5-(3-(2-fluoro-4-(methoxycarbonyl)-5-nitrophenoxy) propoxy)-2-nitrobenzoate (0.35 g, 50.0% yield) as a solid. ¹H NMR (400 MHz, DMSO-d₆) δ 8.19 (dd, J=10.8, 1.3 Hz, 1H), 8.05 (s, 1H), 7.90-7.97 (m, 1H), 7.82 (dd, J=10.9, 1.3 Hz, 1H), 4.40 (q, J=6.2 Hz, 4H), 3.84 (dd, J=12.6, 1.4 Hz, 6H), 2.32 (s, 2H). MS-ESI: m/z 470.0 observed [M+H]⁺.

Step 4: synthesis of methyl 2-amino-5-(3-(5-amino-2-fluoro-4-(methoxycarbonyl) phenoxy)propoxy)-4-fluorobenzoate: To a solution of methyl 4-fluoro-5-(3-(2-fluoro-4-(methoxycarbonyl)-5-nitrophenoxy) propoxy)-2-nitrobenzoate (0.35 g, 0.74 mmol, 1 eq.) in MeOH (7 mL) and THF (7 mL) was added 10% Pd/C catalyst with 50% moist (0.2 g) at rt. The reaction mixture was purged with Hydrogen gas for 1 h. After completion of the reaction, the reaction mixture was filtered on Celite bed and washed with 10% MeOH in DCM solution. The filtrate was concentrated under vacuum to get crude methyl 2-amino-5-(3-(5-amino-2-fluoro-4-(methoxycarbonyl)phenoxy)propoxy)-4-fluorobenzoate (0.30 g, 98.2% yield) which was used in next step without further purification. MS-ESI: m/z 410.0 observed [M+H]⁺.

Step 5: synthesis of methyl 2-(6-(1H-imidazol-1-yl)pyridazine-3-carboxamido)-5-(3-(5-(6-(1H-imidazol-1-yl)pyridazine-3-carboxamido)-2-fluoro-4-(methoxycarbonyl) phenoxy) propoxy)-4-fluorobenzoate: To a stirred solution of intermediate B (0.203 g, 1.073 mmol, 2.2 eq.) in DCE (3 ml) was added DIPEA (0.755 g, 5.85 mmol, 12 eq.) and 50% solution of T₃P (in ethyl acetate) (1.2 g, 3.902 mmol, 8 eq.) at rt. To this, methyl 2-amino-5-(3-(5-amino-2-fluoro-4-(methoxycarbonyl) phenoxy)propoxy)-4-fluorobenzoate (0.200 g, 0.487 mmol, 1 eq.) was added at rt. The reaction mixture was heated at 80-90° C. overnight. After completion of the reaction, the reaction mixture was then directly concentrated under vacuum. The crude material was purified by silica gel column chromatography using 1.5% to 2% MeOH in DCM as a gradient to get pure methyl 2-(6-(1H-imidazol-1-yl)pyridazine-3-carboxamido)-5-(3-(5-(6-(1H-imidazol-1-yl)pyridazine-3-carboxamido)-2-fluoro-4-(methoxycarbonyl)phenoxy) propoxy)-4-fluorobenzoate (0.15 g, 41% yield) as a solid. MS-ESI: m/z 754.0 observed [M+H]⁺.

Step 6: synthesis of 2-(6-(1H-imidazol-1-yl)pyridazine-3-carboxamido)-5-(3-(5-(6-(1H-imidazol-1-yl)pyridazine-3-carboxamido)-4-carboxy-2-fluorophenoxy)propoxy)-4-fluoro-benzoic acid (10): To a solution of methyl 2-(6-(1H-imidazol-1-yl)pyridazine-3-carboxamido)-5-(3-(5-(6-(1H-imidazol-1-yl)pyridazine-3-carboxamido)-2-fluoro-4-(methoxycarbonyl)phenoxy) propoxy)-4-fluorobenzoate (0.15 g, 0.19 mmol, 1 eq.) in can (7.5 mL) and Water (7.5 mL) was added Et₃N (0.25 g, 1.98 mmol, 10 eq) at rt. The reaction mixture was heated in microwave at 120° C. for 5 h. After completion of the reaction, the reaction mixture was directly purified by prep-HPLC without concentration to get compound 10 (0.050 g, 35% yield) as an off-white solid. MS-ESI: m/z 726.17 observed [M+H]⁺.

Step 7: synthesis of lithium 2-(6-(1H-imidazol-1-yl)pyridazine-3-carboxamido)-5-(3-(5-(6-(1H-imidazol-1-yl)pyridazine-3-carboxamido)-4-carboxylato-2-fluorophenoxy)propoxy)-4-fluorobenzoate (10-Li): To a suspension of compound 10 (0.050 g, 0.07 mmol, 1 eq.) in water (4 mL) was added LiOH·H₂O (6 mg, 0.14 mmol, 2.1 eq.) and resultant clear solution was filtered to remove any insoluble particles. The solution was lyophilized to obtain compound 10-Li (0.045 g)¹H NMR (500 MHz, DMSO-d₆) δ 8.78 (s, 2H), 8.71 (d, J=8.2 Hz, 1H), 8.62 (d, J=14.1 Hz, 1H), 8.51-8.37 (m, 4H), 8.19 (s, 2H), 7.81 (dd. J=50.9, 11.2 Hz, 2H), 7.25 (s, 2H), 4.28 (d, J=21.7 Hz, 4H), 2.36 (s, 2H). MS-ESI: m/z 727.2 observed [M+H]⁺.

Procedures analogous to those for the synthesis of compound 10 were used for the synthesis of compounds 26, 27, 31, 33, and 191.

### Example 11: Biological Activity of Compounds

ISRE-luciferase assay. THP-1 Lucia ISG cells were resuspended in low-serum growth media (2% FBS) at a density of 5×10⁵ cells/ml and treated with test article or vehicle (DMSO). 50 μL of cells were seeded into each well of a 384-well white greiner plates and incubated for 24 hours. To evaluate expression of the luciferase reporter, 30 μl of Quanti-luc (Invivogen) detection reagent was added to each well and luminescence was read using an Envision plate reader (Perkin Elmer) set with an integration time of 0.1 seconds. For each cell type, luminescence signals for test article samples were normalized to vehicle-treated samples and reported as relative light units (RLU).

WT STING binding assay (Cisbio, Catalog #64BDSTGPEH). An assay format was optimized to demonstrate binding of recombinant 6×His-tagged human STING protein labeled with Terbium Cryptate by the natural ligand, 2′3′cGAMP labeled with d2 (the acceptor). Upon proximity of the two dyes, the excitation of the donor by the flash lamp on the PHERAstar FSX plate reader triggers a Fluorescence Resonance Energy Transfer (FRET) towards the acceptor, which in turn fluoresces at 665 nm. To assess the ability of the synthetic small molecule STING ligands to bind to human STING, a competitive assay format was applied. A 10-point titration of each of the synthetic ligands in 5 uL were transferred into a 384 well plate, followed by 20 uL of assay buffer containing the 6×His-tagged human STING protein and labeled 2′3′cGAMP ligand and incubated for three hours at room temperature. The raw values obtained from the PHERAstar were used to calculate the reported IC₅₀ values (the signal is inversely proportional to the binding of the synthetic ligand) through curve fitting in Genedata. The percent inhibition was calculated based upon the maximal amount of binding by synthetic compound versus the maximum binding of unlabeled 2′3′ cGAMP which was used as a control in each assay.

Assay results for selected representative compounds of the present disclosure are presented in Table 2. The results were scored as follows:

Description

| Score | ISRE-Luc Assay | STING-binding HTRF assay |
|---|---|---|
| − | not active | not active |
| + | compound induces greater than or | compound induces greater than or |
| | equal to a 20% increase in ISRE | equal to a 20% decrease in |
| | reporter signal and EC50 is | binding of labeled STING ligand |
| | greater than 5 μM | and calculated IC50 is greater |
| | | than 1 μM |
| ++ | compound induces greater than or | compound induces greater than or |
| | equal to a 20% increase in ISRE | equal to a 20% decrease in |
| | reporter signal and calculated | binding of labeled STING ligand |
| | EC50 is between 0.5 and 5 μM | and calculated IC50 is between |
| | | 0.1 and 1 μM |
| +++ | compound induces greater than or | compound induces greater than or |
| | equal to a 20% increase in ISRE | equal to a 20% decrease in |
| | reporter signal and calculated | binding of labeled STING ligand |
| | EC50 is between 0.05 and 0.5 μM | and calculated IC50 is between |
| | | 0.01 and 0.1 μM |
| ++++ | compound induces greater than or | compound induces greater than or |
| | equal to a 20% increase in ISRE | equal to a 20% decrease in |
| | reporter signal and calculated | binding of labeled STING ligand |
| | EC50 is less than 0.05 μM | and calculated IC50 is less than |
| | | 0.01 μM |

TABLE 2
Results of ISRE-Luc and STING-binding HTRF Assays.

| | | ISG-LUC | Human |
|---|---|---|---|
| | | activation | STING |
| Com- | | assay | Binding |
| pound | Structure | (EC50) | (IC50) |
| 1 | 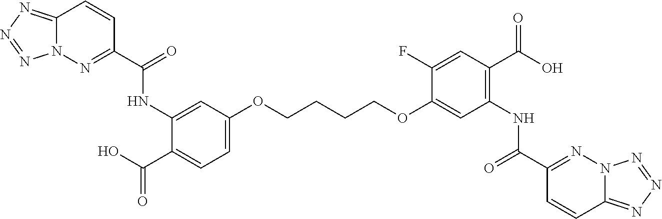 [SMILES: O=C(Nc1cc(OCCCCOc2cc(NC(=O)c3ccc4nnnn4n3)c(C(=O)O)cc2F)ccc1C(=O)O)c1ccc2nnnn2n1] | ++ | ++ |
| 2 | 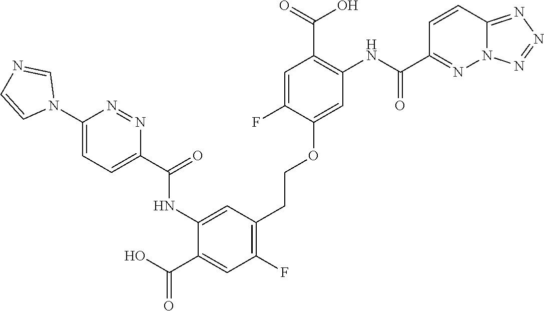 [SMILES: O=C(Nc1cc(CCOc2cc(NC(=O)c3ccc4nnnn4n3)c(C(=O)O)cc2F)c(F)cc1C(=O)O)c1ccc(-n2ccnc2)nn1] | ++++ | +++ |
| 3 | 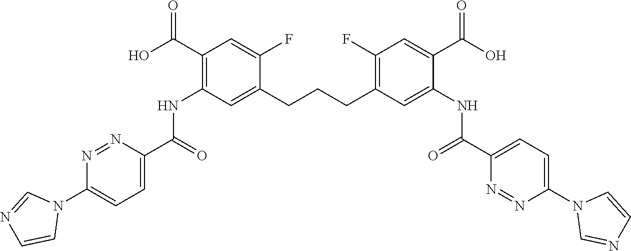 [SMILES: O=C(Nc1cc(CCCc2cc(NC(=O)c3ccc(-n4ccnc4)nn3)c(C(=O)O)cc2F)c(F)cc1C(=O)O)c1ccc(-n2ccnc2)nn1] | +++ | ++++ |
| 4 | 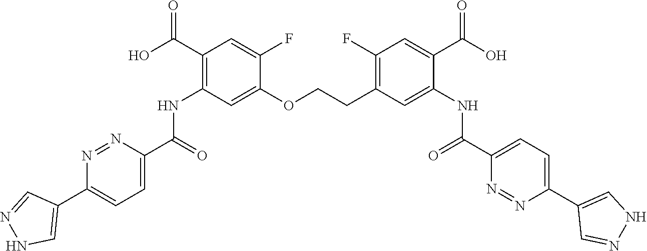 [SMILES: O=C(Nc1cc(CCOc2cc(NC(=O)c3ccc(-c4cn[nH]c4)nn3)c(C(=O)O)cc2F)c(F)cc1C(=O)O)c1ccc(-c2cn[nH]c2)nn1] | +++ | ++++ |
| 5 | 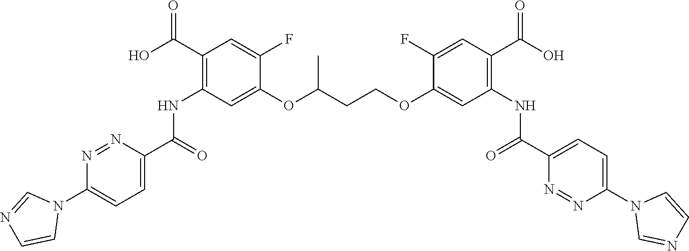 [SMILES: CC(CCOc1cc(NC(=O)c2ccc(-n3ccnc3)nn2)c(C(=O)O)cc1F)Oc1cc(NC(=O)c2ccc(-n3ccnc3)nn2)c(C(=O)O)cc1F] | +++ | ++++ |
| 6 | 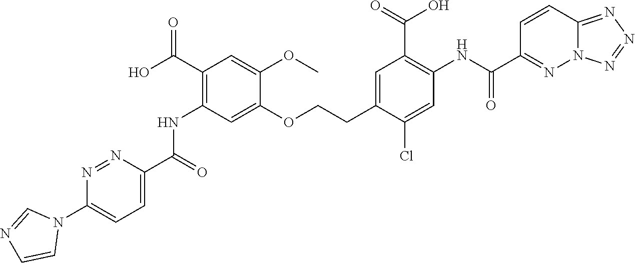 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1Cl] | +++ | ++++ |
| 7 | 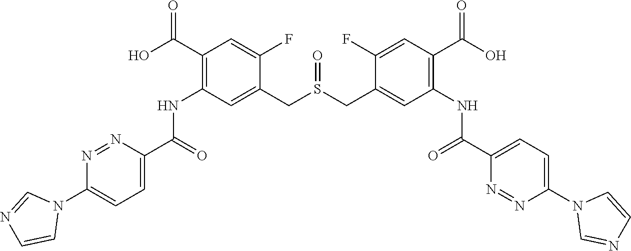 [SMILES: O=C(Nc1cc(CS(=O)Cc2cc(NC(=O)c3ccc(-n4ccnc4)nn3)c(C(=O)O)cc2F)c(F)cc1C(=O)O)c1ccc(-n2ccnc2)nn1] | ++ | +++ |
| 8 | 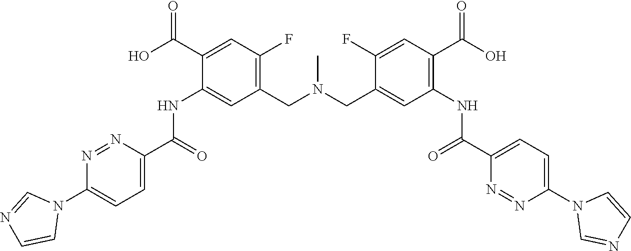 [SMILES: CN(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] | ++ | ++++ |
| 9 | 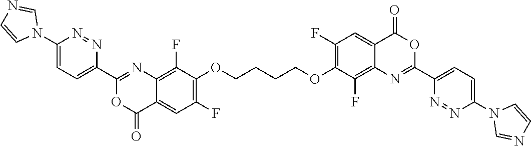 [SMILES: O=c1oc(-c2ccc(-n3ccnc3)nn2)nc2c(F)c(OCCCCOc3c(F)cc4c(=O)oc(-c5ccc(-n6ccnc6)nn5)nc4c3F)c(F)cc12] | ++ | − |
| 10 | 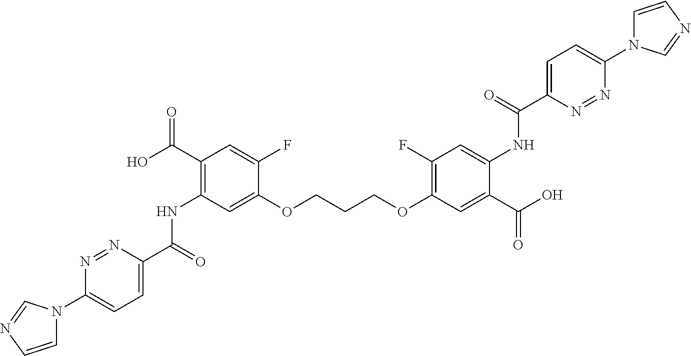 [SMILES: O=C(Nc1cc(OCCCOc2cc(C(=O)O)c(NC(=O)c3ccc(-n4ccnc4)nn3)cc2F)c(F)cc1C(=O)O)c1ccc(-n2ccnc2)nn1] | +++ | ++++ |
| 11 | 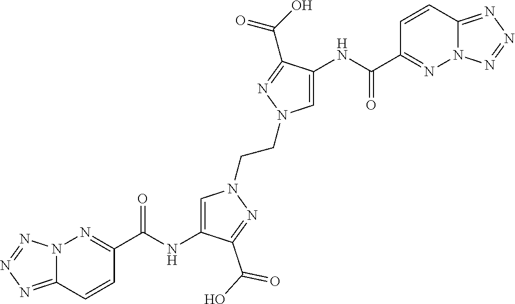 [SMILES: O=C(Nc1cn(CCn2cc(NC(=O)c3ccc4nnnn4n3)c(C(=O)O)n2)nc1C(=O)O)c1ccc2nnnn2n1] | − | − |
| 12 | 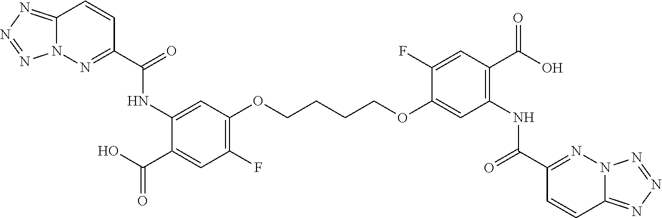 [SMILES: O=C(Nc1cc(OCCCCOc2cc(NC(=O)c3ccc4nnnn4n3)c(C(=O)O)cc2F)c(F)cc1C(=O)O)c1ccc2nnnn2n1] | +++ | ++ |
| 13 | 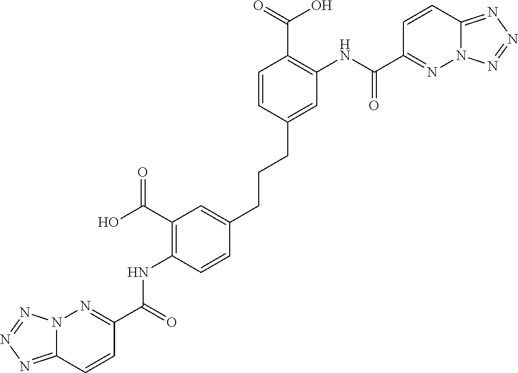 [SMILES: O=C(Nc1cc(CCCc2ccc(NC(=O)c3ccc4nnnn4n3)c(C(=O)O)c2)ccc1C(=O)O)c1ccc2nnnn2n1] | ++ | + |
| 14 | 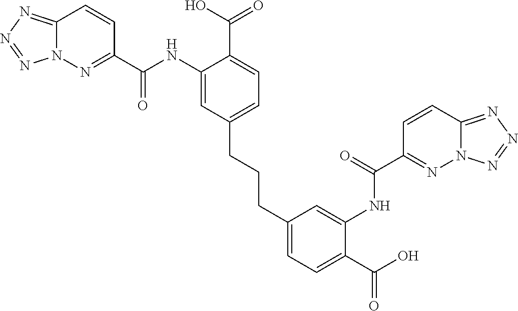 [SMILES: O=C(Nc1cc(CCCc2ccc(C(=O)O)c(NC(=O)c3ccc4nnnn4n3)c2)ccc1C(=O)O)c1ccc2nnnn2n1] | ++ | + |
| 15 | 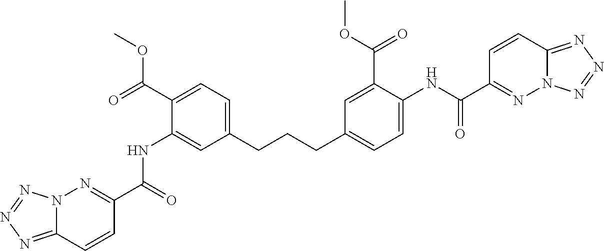 [SMILES: COC(=O)c1ccc(CCCc2ccc(NC(=O)c3ccc4nnnn4n3)c(C(=O)OC)c2)cc1NC(=O)c1ccc2nnnn2n1] | − | |
| 16 | 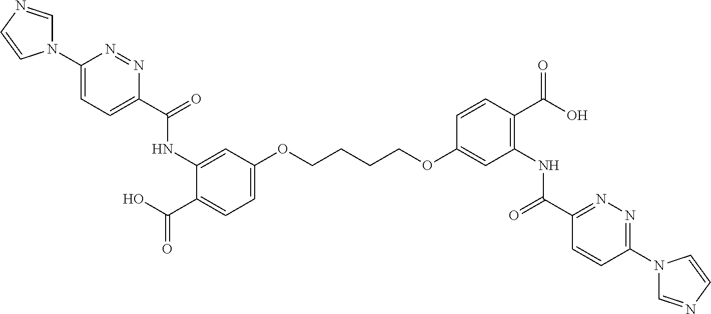 [SMILES: O=C(Nc1cc(OCCCCOc2ccc(C(=O)O)c(NC(=O)c3ccc(-n4ccnc4)nn3)c2)ccc1C(=O)O)c1ccc(-n2ccnc2)nn1] | + | ++ |
| 17 | 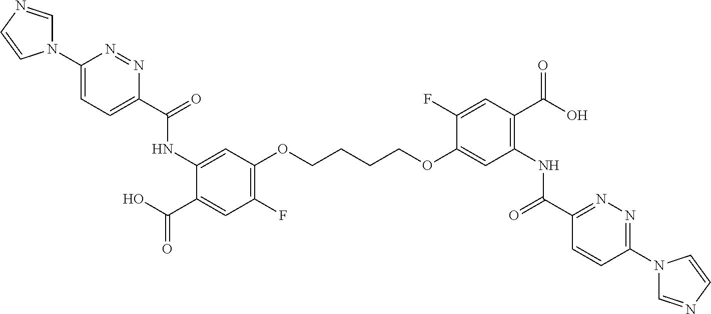 [SMILES: O=C(Nc1cc(OCCCCOc2cc(NC(=O)c3ccc(-n4ccnc4)nn3)c(C(=O)O)cc2F)c(F)cc1C(=O)O)c1ccc(-n2ccnc2)nn1] | ++ | ++ |
| 18 | 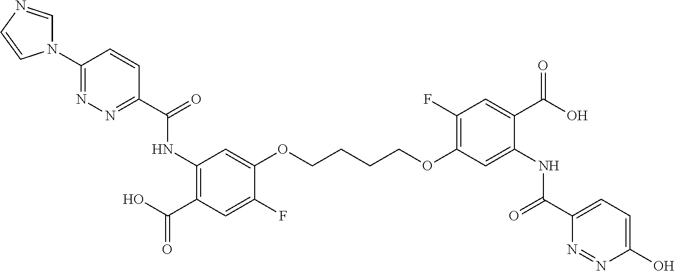 [SMILES: O=C(Nc1cc(OCCCCOc2cc(NC(=O)c3ccc(-n4ccnc4)nn3)c(C(=O)O)cc2F)c(F)cc1C(=O)O)c1ccc(O)nn1] | + | ++ |
| 19 | 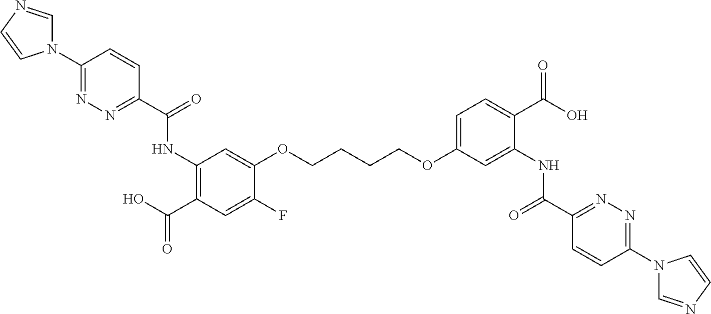 [SMILES: O=C(Nc1cc(OCCCCOc2cc(NC(=O)c3ccc(-n4ccnc4)nn3)c(C(=O)O)cc2F)ccc1C(=O)O)c1ccc(-n2ccnc2)nn1] | ++ | ++++ |
| 20 | 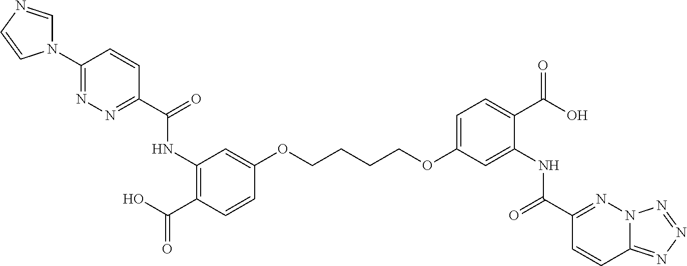 [SMILES: O=C(Nc1cc(OCCCCOc2ccc(C(=O)O)c(NC(=O)c3ccc4nnnn4n3)c2)ccc1C(=O)O)c1ccc(-n2ccnc2)nn1] | + | ++ |
| 21 | 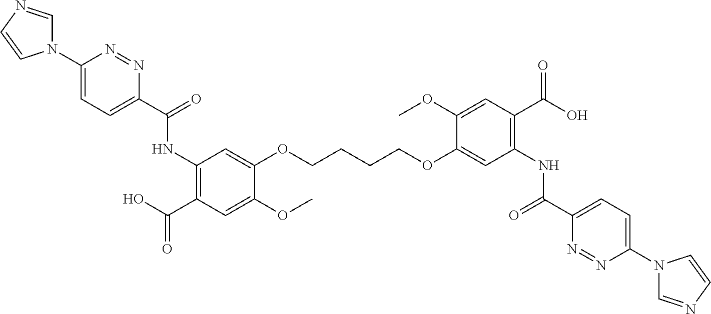 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1OC] | − | + |
| 22 | 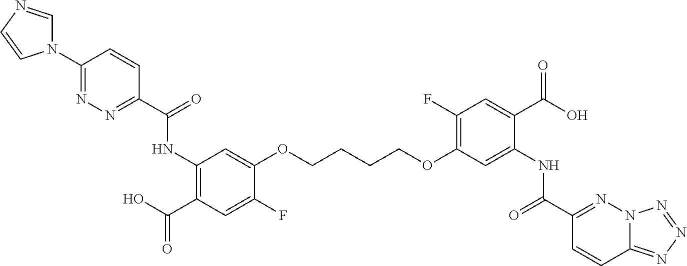 [SMILES: O=C(Nc1cc(OCCCCOc2cc(NC(=O)c3ccc4nnnn4n3)c(C(=O)O)cc2F)c(F)cc1C(=O)O)c1ccc(-n2ccnc2)nn1] | + | ++ |
| 23 | 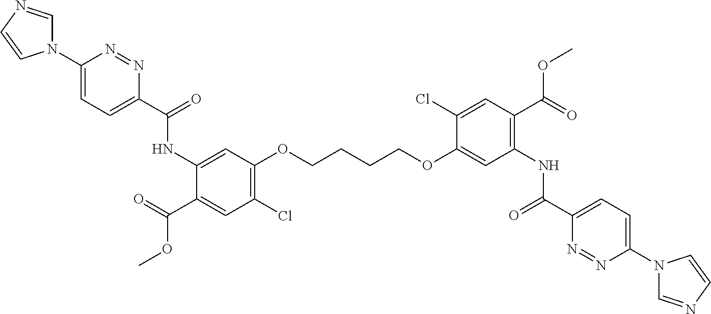 [SMILES: COC(=O)c1cc(Cl)c(OCCCCOc2cc(NC(=O)c3ccc(-n4ccnc4)nn3)c(C(=O)OC)cc2Cl)cc1NC(=O)c1ccc(-n2ccnc2)nn1] | ++++ | |
| 24 | 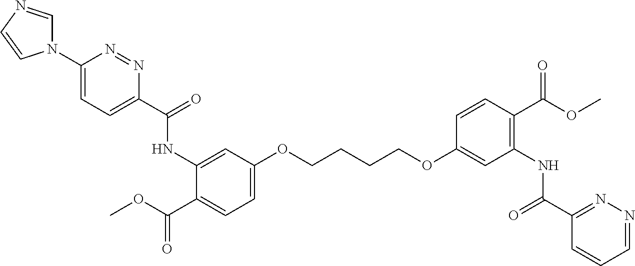 [SMILES: COC(=O)c1ccc(OCCCCOc2ccc(C(=O)OC)c(NC(=O)c3ccc(-n4ccnc4)nn3)c2)cc1NC(=O)c1cccnn1] | − | |
| 25 | 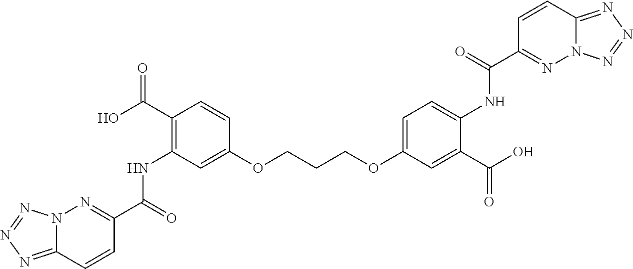 [SMILES: O=C(Nc1cc(OCCCOc2ccc(NC(=O)c3ccc4nnnn4n3)c(C(=O)O)c2)ccc1C(=O)O)c1ccc2nnnn2n1] | − | + |
| 26 | 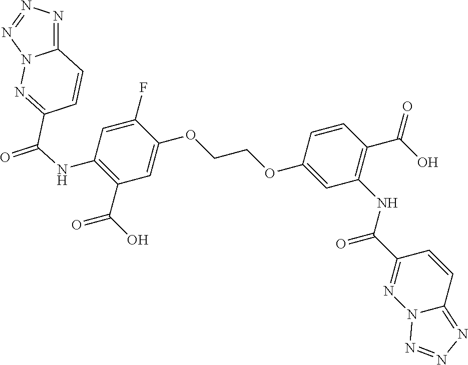 [SMILES: O=C(Nc1cc(OCCOc2cc(C(=O)O)c(NC(=O)c3ccc4nnnn4n3)cc2F)ccc1C(=O)O)c1ccc2nnnn2n1] | + | + |
| 27 | 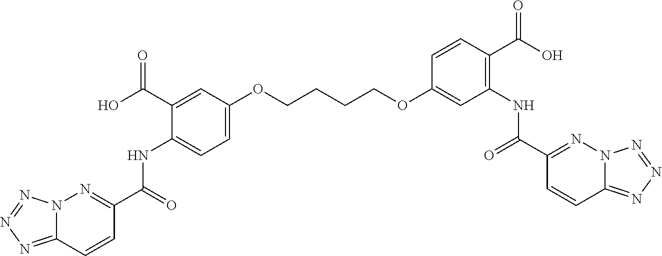 [SMILES: O=C(Nc1cc(OCCCCOc2ccc(NC(=O)c3ccc4nnnn4n3)c(C(=O)O)c2)ccc1C(=O)O)c1ccc2nnnn2n1] | − | + |
| 28 | 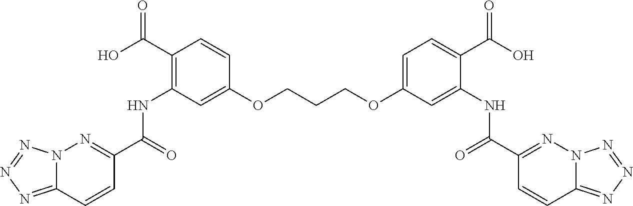 [SMILES: O=C(Nc1cc(OCCCOc2ccc(C(=O)O)c(NC(=O)c3ccc4nnnn4n3)c2)ccc1C(=O)O)c1ccc2nnnn2n1] | − | + |
| 29 | 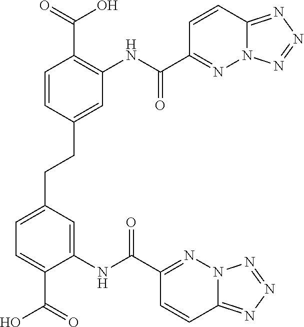 [SMILES: O=C(Nc1cc(CCc2ccc(C(=O)O)c(NC(=O)c3ccc4nnnn4n3)c2)ccc1C(=O)O)c1ccc2nnnn2n1] | − | + |
| 30 | 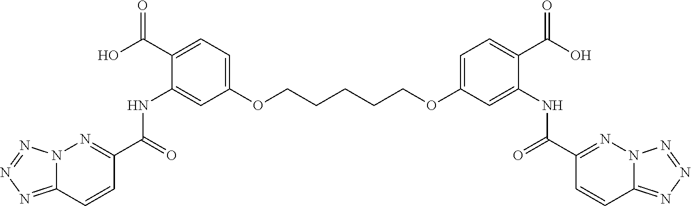 [SMILES: O=C(Nc1cc(OCCCCCOc2ccc(C(=O)O)c(NC(=O)c3ccc4nnnn4n3)c2)ccc1C(=O)O)c1ccc2nnnn2n1] | − | + |
| 31 | 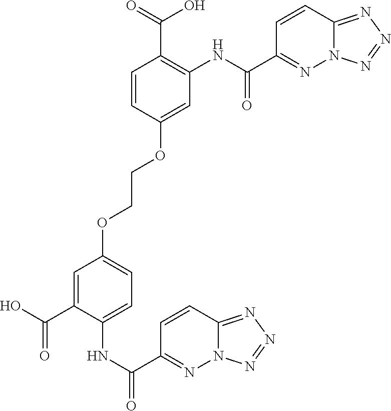 [SMILES: O=C(Nc1cc(OCCOc2ccc(NC(=O)c3ccc4nnnn4n3)c(C(=O)O)c2)ccc1C(=O)O)c1ccc2nnnn2n1] | − | + |
| 32 | 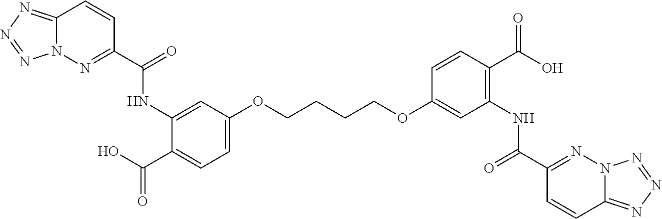 [SMILES: O=C(Nc1cc(OCCCCOc2ccc(C(=O)O)c(NC(=O)c3ccc4nnnn4n3)c2)ccc1C(=O)O)c1ccc2nnnn2n1] | − | + |
| 33 | 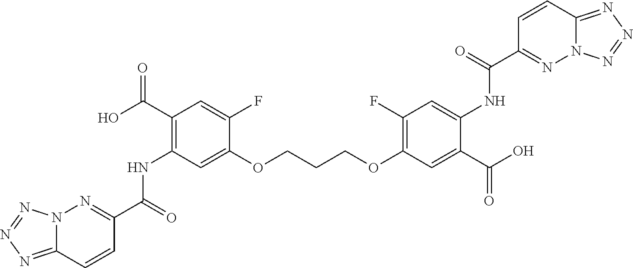 [SMILES: O=C(Nc1cc(OCCCOc2cc(C(=O)O)c(NC(=O)c3ccc4nnnn4n3)cc2F)c(F)cc1C(=O)O)c1ccc2nnnn2n1] | + | + |
| 34 | 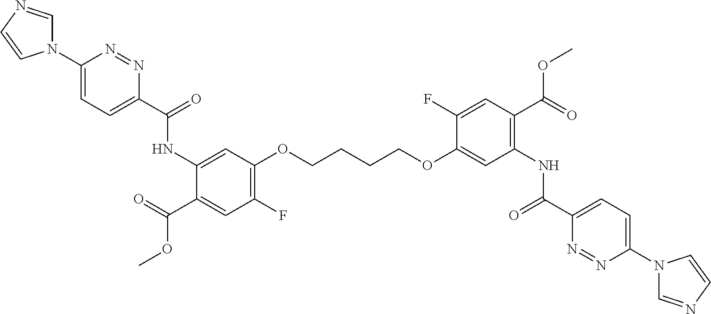 [SMILES: COC(=O)c1cc(F)c(OCCCCOc2cc(NC(=O)c3ccc(-n4ccnc4)nn3)c(C(=O)OC)cc2F)cc1NC(=O)c1ccc(-n2ccnc2)nn1] | + | |
| 35 | 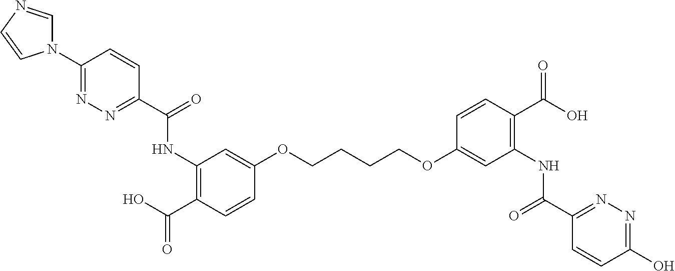 [SMILES: O=C(Nc1cc(OCCCCOc2ccc(C(=O)O)c(NC(=O)c3ccc(-n4ccnc4)nn3)c2)ccc1C(=O)O)c1ccc(O)nn1] | + | + |
| 36 | 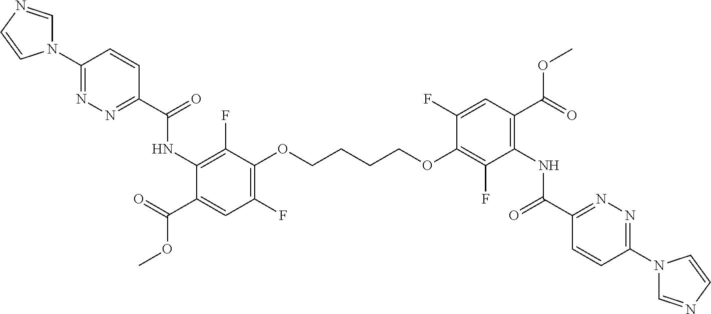 [SMILES: COC(=O)c1cc(F)c(OCCCCOc2c(F)cc(C(=O)OC)c(NC(=O)c3ccc(-n4ccnc4)nn3)c2F)c(F)c1NC(=O)c1ccc(-n2ccnc2)nn1] | ++++ | |
| 37 | 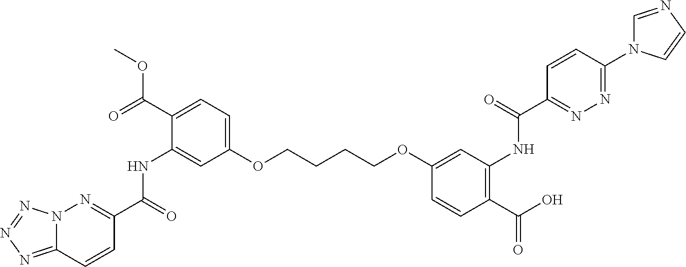 [SMILES: COC(=O)c1ccc(OCCCCOc2ccc(C(=O)O)c(NC(=O)c3ccc(-n4ccnc4)nn3)c2)cc1NC(=O)c1ccc2nnnn2n1] | − | + |
| 38 | 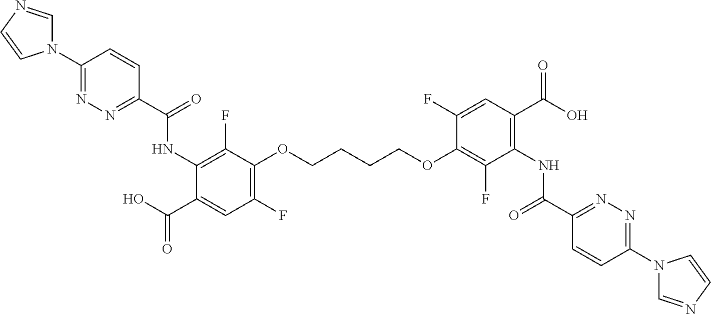 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(-n2ccnc2)nn1] | +++ | + |
| 39 | 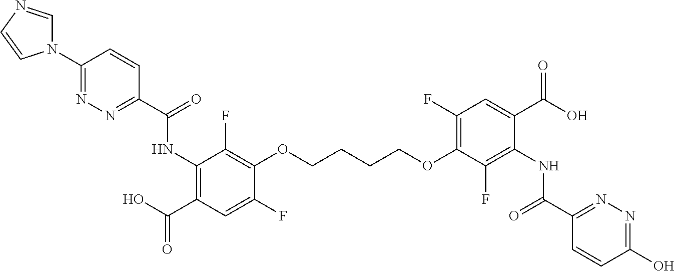 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(O)nn1] | + | + |
| 40 | 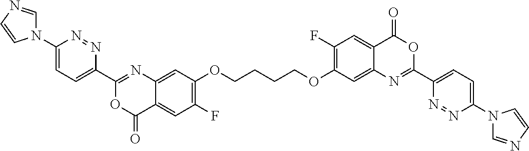 [SMILES: O=c1oc(-c2ccc(-n3ccnc3)nn2)nc2cc(OCCCCOc3cc4nc(-c5ccc(-n6ccnc6)nn5)oc(=O)c4cc3F)c(F)cc12] | ++++ | − |
| 41 | 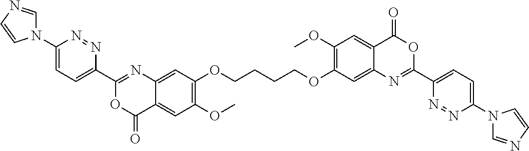 [SMILES: COc1cc2c(=O)oc(-c3ccc(-n4ccnc4)nn3)nc2cc1OCCCCOc1cc2nc(-c3ccc(-n4ccnc4)nn3)oc(=O)c2cc1OC] | ++++ | |
| 42 | 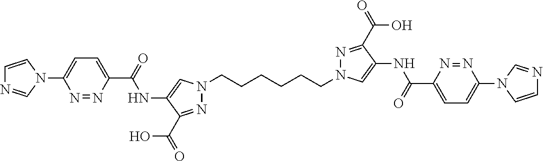 [SMILES: O=C(Nc1cn(CCCCCCn2cc(NC(=O)c3ccc(-n4ccnc4)nn3)c(C(=O)O)n2)nc1C(=O)O)c1ccc(-n2ccnc2)nn1] | − | − |
| 43 | 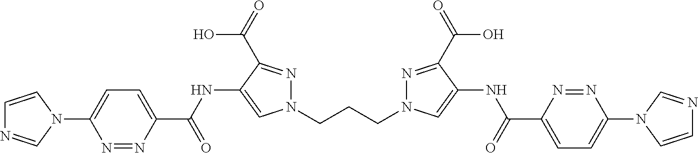 [SMILES: O=C(Nc1cn(CCCn2cc(NC(=O)c3ccc(-n4ccnc4)nn3)c(C(=O)O)n2)nc1C(=O)O)c1ccc(-n2ccnc2)nn1] | − | − |
| 44 | 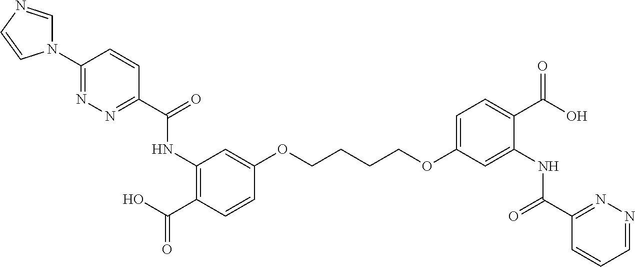 [SMILES: O=C(Nc1cc(OCCCCOc2ccc(C(=O)O)c(NC(=O)c3ccc(-n4ccnc4)nn3)c2)ccc1C(=O)O)c1cccnn1] | + | − |
| 45 | 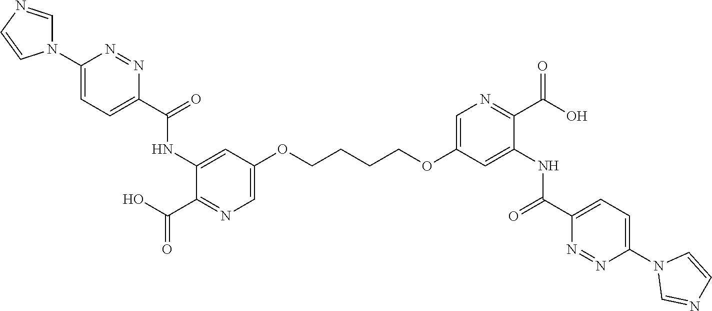 [SMILES: O=C(Nc1cc(OCCCCOc2cnc(C(=O)O)c(NC(=O)c3ccc(-n4ccnc4)nn3)c2)cnc1C(=O)O)c1ccc(-n2ccnc2)nn1] | + | + |
| 46 | 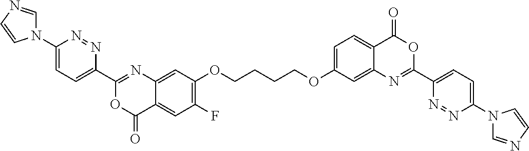 [SMILES: O=c1oc(-c2ccc(-n3ccnc3)nn2)nc2cc(OCCCCOc3cc4nc(-c5ccc(-n6ccnc6)nn5)oc(=O)c4cc3F)ccc12] | ++ | ++ |
| 47 | 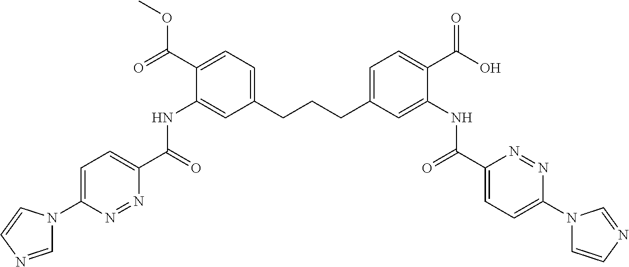 [SMILES: COC(=O)c1ccc(CCCc2ccc(C(=O)O)c(NC(=O)c3ccc(-n4ccnc4)nn3)c2)cc1NC(=O)c1ccc(-n2ccnc2)nn1] | ++ | ++ |
| 48 | 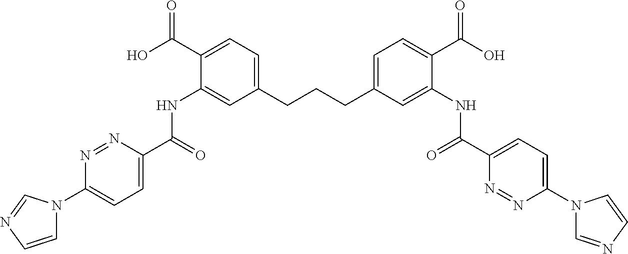 [SMILES: O=C(Nc1cc(CCCc2ccc(C(=O)O)c(NC(=O)c3ccc(-n4ccnc4)nn3)c2)ccc1C(=O)O)c1ccc(-n2ccnc2)nn1] | +++ | +++ |
| 49 | 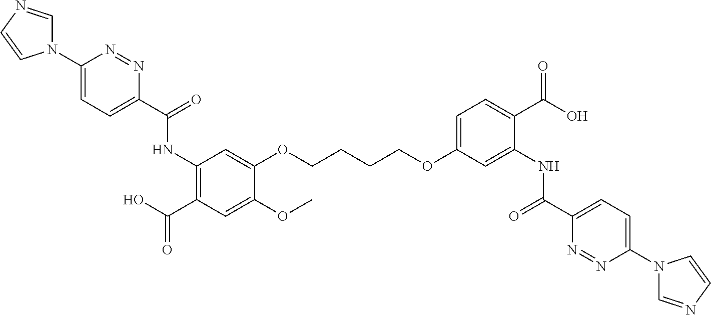 [SMILES: COc1cc(C(=O)O)c(NC(=O)c2ccc(-n3ccnc3)nn2)cc1OCCCCOc1ccc(C(=O)O)c(NC(=O)c2ccc(-n3ccnc3)nn2)c1] | + | ++ |
| 50 | 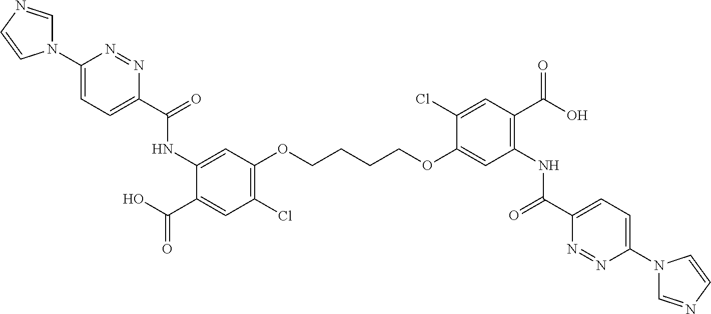 [SMILES: O=C(Nc1cc(OCCCCOc2cc(NC(=O)c3ccc(-n4ccnc4)nn3)c(C(=O)O)cc2Cl)c(Cl)cc1C(=O)O)c1ccc(-n2ccnc2)nn1] | ++ | ++ |
| 51 | 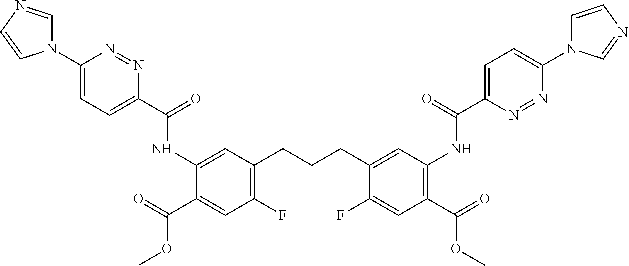 [SMILES: COC(=O)c1cc(F)c(CCCc2cc(NC(=O)c3ccc(-n4ccnc4)nn3)c(C(=O)OC)cc2F)cc1NC(=O)c1ccc(-n2ccnc2)nn1] | ++++ | |
| 52 | 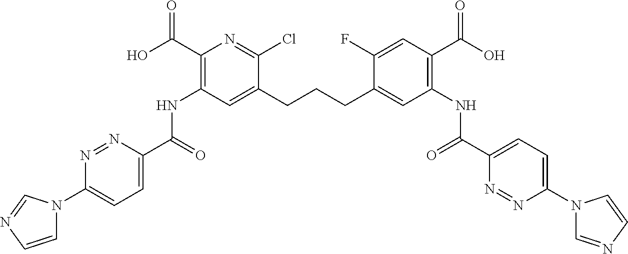 [SMILES: O=C(Nc1cc(CCCc2cc(NC(=O)c3ccc(-n4ccnc4)nn3)c(C(=O)O)nc2Cl)c(F)cc1C(=O)O)c1ccc(-n2ccnc2)nn1] | +++ | +++ |
| 53 | 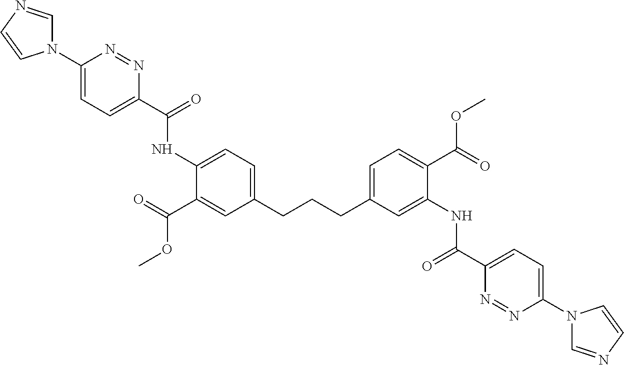 [SMILES: COC(=O)c1ccc(CCCc2ccc(NC(=O)c3ccc(-n4ccnc4)nn3)c(C(=O)OC)c2)cc1NC(=O)c1ccc(-n2ccnc2)nn1] | + | |
| 54 | 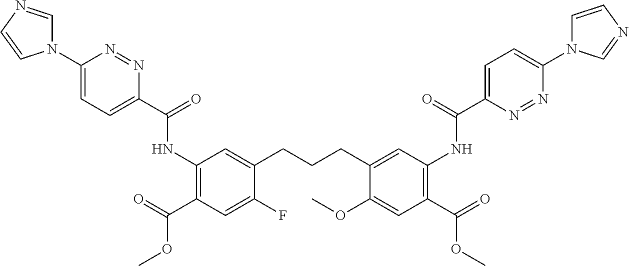 [SMILES: COC(=O)c1cc(F)c(CCCc2cc(NC(=O)c3ccc(-n4ccnc4)nn3)c(C(=O)OC)cc2OC)cc1NC(=O)c1ccc(-n2ccnc2)nn1] | ++++ | |
| 55 | 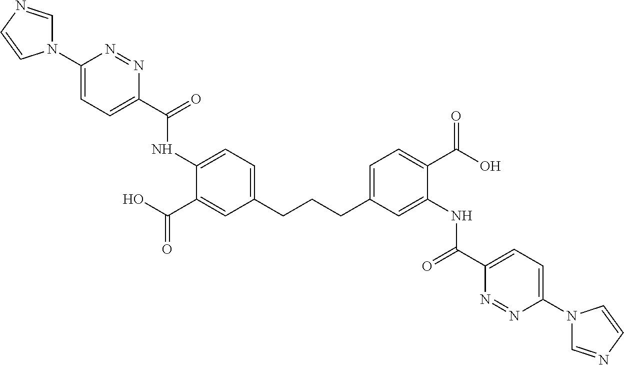 [SMILES: O=C(Nc1cc(CCCc2ccc(NC(=O)c3ccc(-n4ccnc4)nn3)c(C(=O)O)c2)ccc1C(=O)O)c1ccc(-n2ccnc2)nn1] | ++ | +++ |
| 56 | 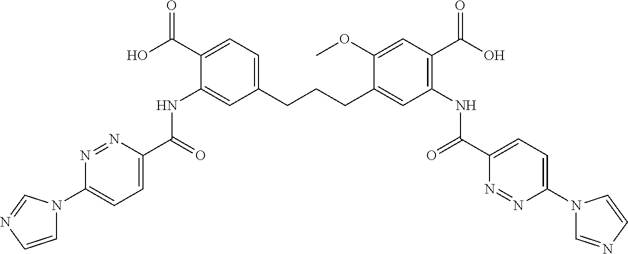 [SMILES: COc1cc(C(=O)O)c(NC(=O)c2ccc(-n3ccnc3)nn2)cc1CCCc1ccc(C(=O)O)c(NC(=O)c2ccc(-n3ccnc3)nn2)c1] | +++ | ++++ |
| 57 | 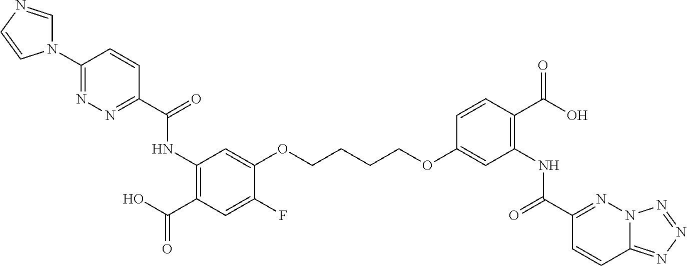 [SMILES: O=C(Nc1cc(OCCCCOc2ccc(C(=O)O)c(NC(=O)c3ccc4nnnn4n3)c2)c(F)cc1C(=O)O)c1ccc(-n2ccnc2)nn1] | + | +++ |
| 58 | 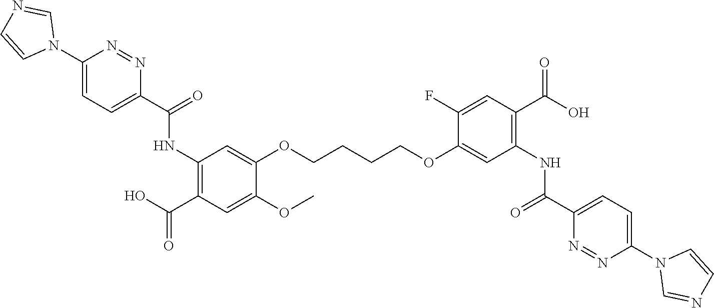 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1F] | ++ | ++ |
| 59 | 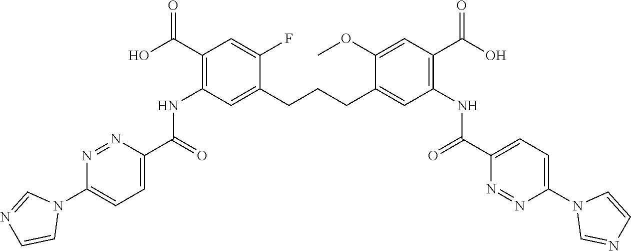 [SMILES: COc1cc(C(=O)O)c(NC(=O)c2ccc(-n3ccnc3)nn2)cc1CCCc1cc(NC(=O)c2ccc(-n3ccnc3)nn2)c(C(=O)O)cc1F] | +++ | ++++ |
| 60 | 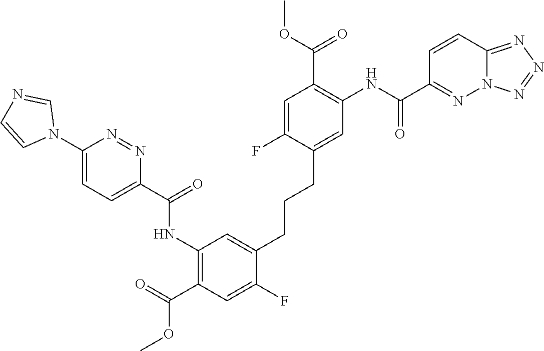 [SMILES: COC(=O)c1cc(F)c(CCCc2cc(NC(=O)c3ccc4nnnn4n3)c(C(=O)OC)cc2F)cc1NC(=O)c1ccc(-n2ccnc2)nn1] | +++ | |
| 61 | 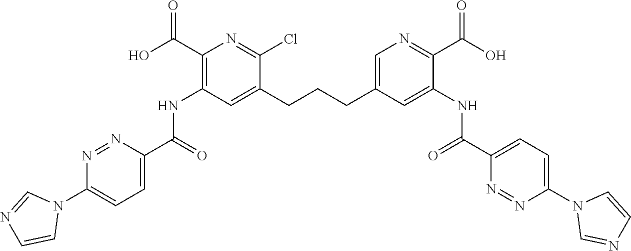 [SMILES: O=C(Nc1cc(CCCc2cc(NC(=O)c3ccc(-n4ccnc4)nn3)c(C(=O)O)nc2Cl)cnc1C(=O)O)c1ccc(-n2ccnc2)nn1] | ++ | ++ |
| 62 | 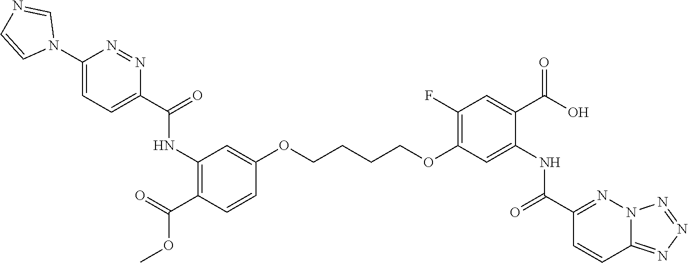 [SMILES: COC(=O)c1ccc(OCCCCOc2cc(NC(=O)c3ccc4nnnn4n3)c(C(=O)O)cc2F)cc1NC(=O)c1ccc(-n2ccnc2)nn1] | + | ++ |
| 63 | 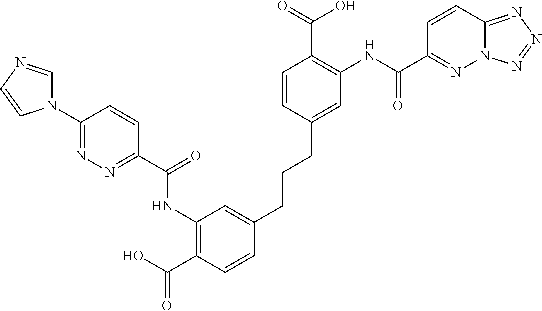 [SMILES: O=C(Nc1cc(CCCc2ccc(C(=O)O)c(NC(=O)c3ccc4nnnn4n3)c2)ccc1C(=O)O)c1ccc(-n2ccnc2)nn1] | + | + |
| 64 | 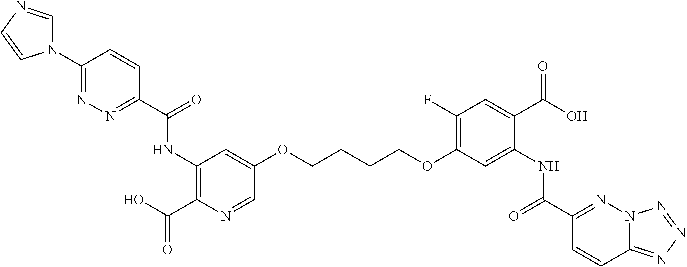 [SMILES: O=C(Nc1cc(OCCCCOc2cnc(C(=O)O)c(NC(=O)c3ccc(-n4ccnc4)nn3)c2)c(F)cc1C(=O)O)c1ccc2nnnn2n1] | + | +++ |
| 65 | 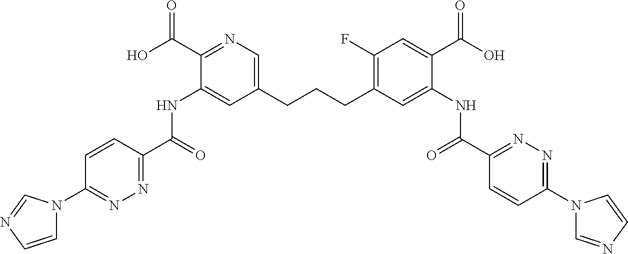 [SMILES: O=C(Nc1cc(CCCc2cnc(C(=O)O)c(NC(=O)c3ccc(-n4ccnc4)nn3)c2)c(F)cc1C(=O)O)c1ccc(-n2ccnc2)nn1] | ++ | +++ |
| 66 | 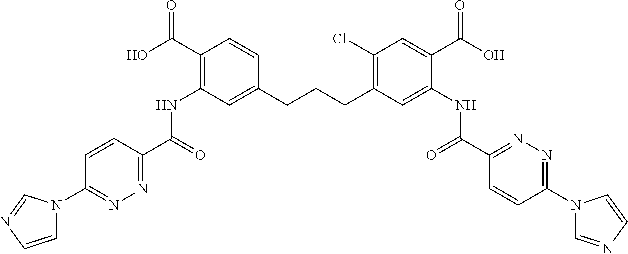 [SMILES: O=C(Nc1cc(CCCc2cc(NC(=O)c3ccc(-n4ccnc4)nn3)c(C(=O)O)cc2Cl)ccc1C(=O)O)c1ccc(-n2ccnc2)nn1] | +++ | ++++ |
| 67 | 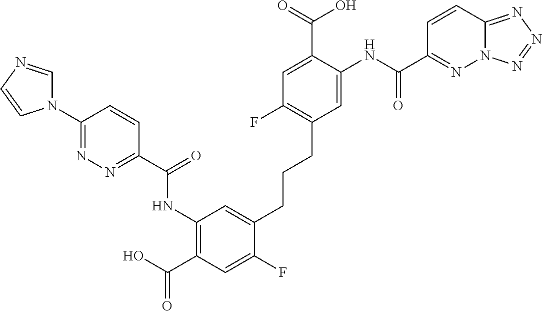 [SMILES: O=C(Nc1cc(CCCc2cc(NC(=O)c3ccc4nnnn4n3)c(C(=O)O)cc2F)c(F)cc1C(=O)O)c1ccc(-n2ccnc2)nn1] | +++ | ++++ |
| 68 | 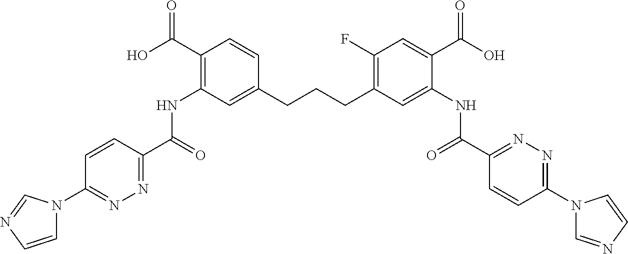 [SMILES: O=C(Nc1cc(CCCc2cc(NC(=O)c3ccc(-n4ccnc4)nn3)c(C(=O)O)cc2F)ccc1C(=O)O)c1ccc(-n2ccnc2)nn1] | +++ | ++++ |
| 69 | 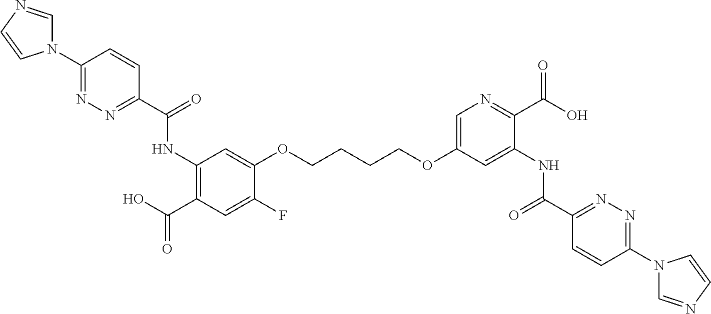 [SMILES: O=C(Nc1cc(OCCCCOc2cnc(C(=O)O)c(NC(=O)c3ccc(-n4ccnc4)nn3)c2)c(F)cc1C(=O)O)c1ccc(-n2ccnc2)nn1] | − | ++ |
| 70 | 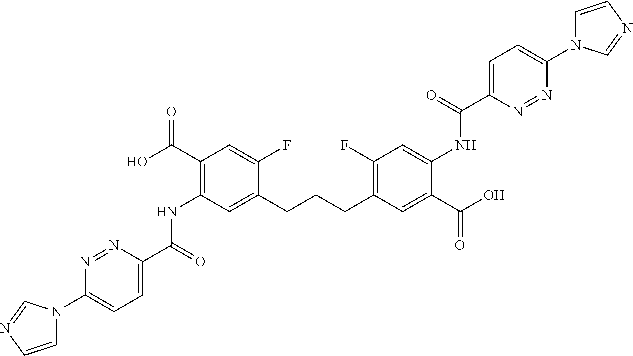 [SMILES: O=C(Nc1cc(CCCc2cc(C(=O)O)c(NC(=O)c3ccc(-n4ccnc4)nn3)cc2F)c(F)cc1C(=O)O)c1ccc(-n2ccnc2)nn1] | ++ | ++++ |
| 71 | 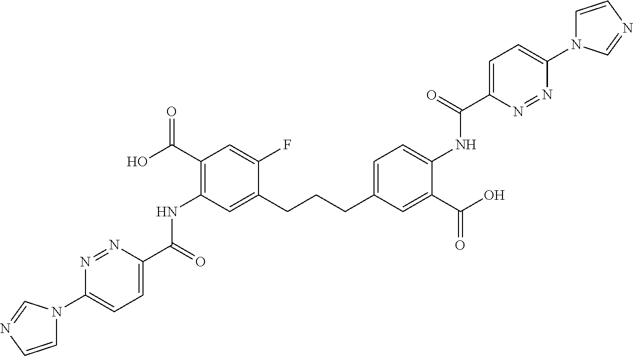 [SMILES: O=C(Nc1ccc(CCCc2cc(NC(=O)c3ccc(-n4ccnc4)nn3)c(C(=O)O)cc2F)cc1C(=O)O)c1ccc(-n2ccnc2)nn1] | + | +++ |
| 72 | 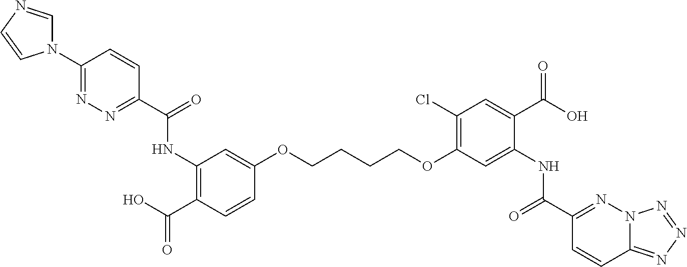 [SMILES: O=C(Nc1cc(OCCCCOc2cc(NC(=O)c3ccc4nnnn4n3)c(C(=O)O)cc2Cl)ccc1C(=O)O)c1ccc(-n2ccnc2)nn1] | ++ | +++ |
| 73 | 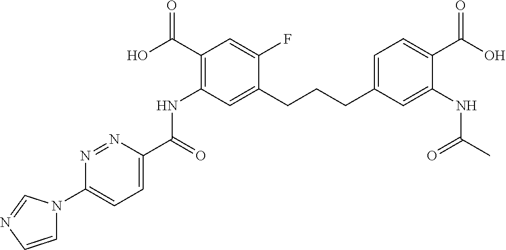 [SMILES: CC(=O)Nc1cc(CCCc2cc(NC(=O)c3ccc(-n4ccnc4)nn3)c(C(=O)O)cc2F)ccc1C(=O)O] | − | ++ |
| 74 | 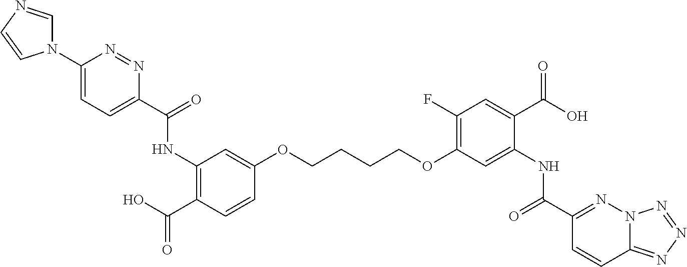 [SMILES: O=C(Nc1cc(OCCCCOc2cc(NC(=O)c3ccc4nnnn4n3)c(C(=O)O)cc2F)ccc1C(=O)O)c1ccc(-n2ccnc2)nn1] | + | +++ |
| 75 | 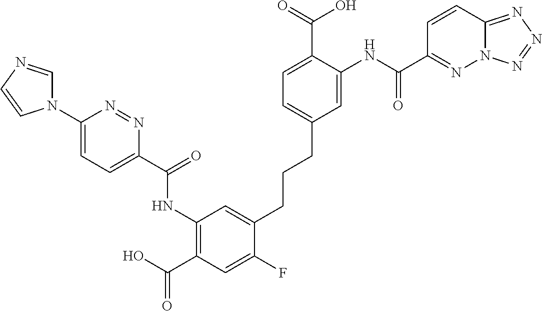 [SMILES: O=C(Nc1cc(CCCc2ccc(C(=O)O)c(NC(=O)c3ccc4nnnn4n3)c2)c(F)cc1C(=O)O)c1ccc(-n2ccnc2)nn1] | ++ | ++ |
| 76 | 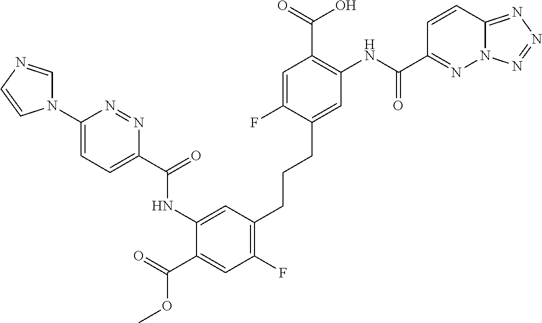 [SMILES: COC(=O)c1cc(F)c(CCCc2cc(NC(=O)c3ccc4nnnn4n3)c(C(=O)O)cc2F)cc1NC(=O)c1ccc(-n2ccnc2)nn1] | +++ | +++ |
| 77 | 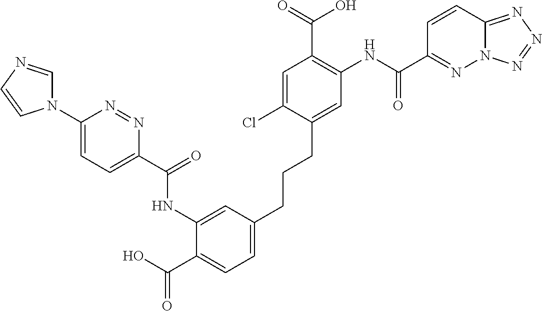 [SMILES: O=C(Nc1cc(CCCc2cc(NC(=O)c3ccc4nnnn4n3)c(C(=O)O)cc2Cl)ccc1C(=O)O)c1ccc(-n2ccnc2)nn1] | +++ | +++ |
| 78 | 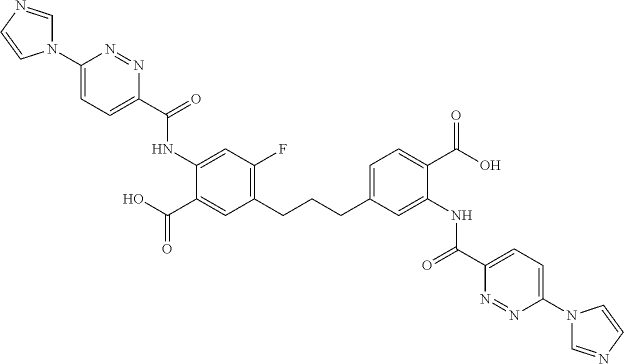 [SMILES: O=C(Nc1cc(CCCc2cc(C(=O)O)c(NC(=O)c3ccc(-n4ccnc4)nn3)cc2F)ccc1C(=O)O)c1ccc(-n2ccnc2)nn1] | +++ | ++++ |
| 79 | 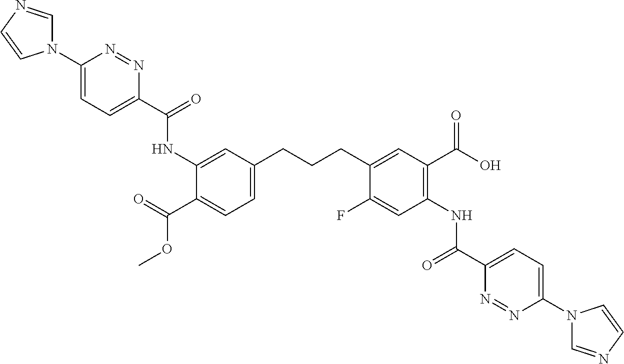 [SMILES: COC(=O)c1ccc(CCCc2cc(C(=O)O)c(NC(=O)c3ccc(-n4ccnc4)nn3)cc2F)cc1NC(=O)c1ccc(-n2ccnc2)nn1] | +++ | ++ |
| 80 | 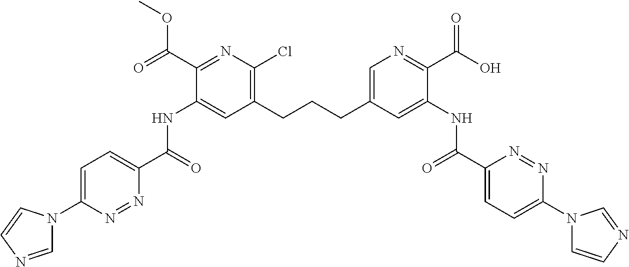 [SMILES: COC(=O)c1nc(Cl)c(CCCc2cnc(C(=O)O)c(NC(=O)c3ccc(-n4ccnc4)nn3)c2)cc1NC(=O)c1ccc(-n2ccnc2)nn1] | ++ | +++ |
| 81 | 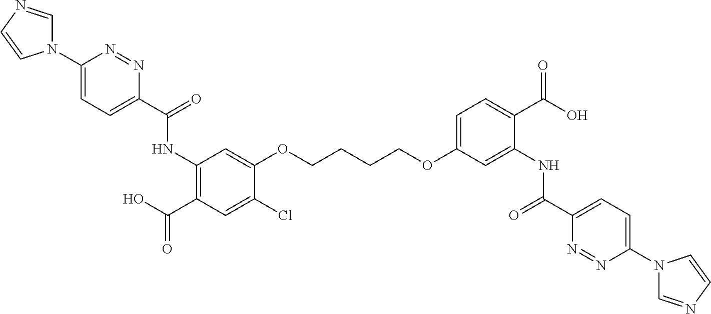 [SMILES: O=C(Nc1cc(OCCCCOc2cc(NC(=O)c3ccc(-n4ccnc4)nn3)c(C(=O)O)cc2Cl)ccc1C(=O)O)c1ccc(-n2ccnc2)nn1] | − | ++ |
| 82 | 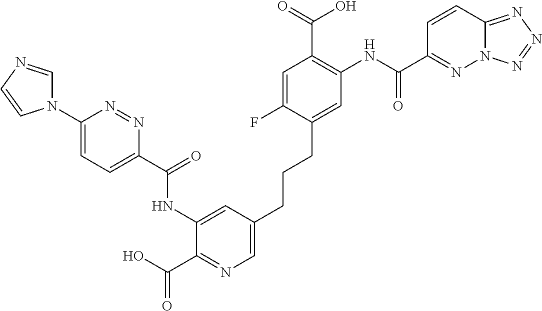 [SMILES: O=C(Nc1cc(CCCc2cnc(C(=O)O)c(NC(=O)c3ccc(-n4ccnc4)nn3)c2)c(F)cc1C(=O)O)c1ccc2nnnn2n1] | + | ++ |
| 83 | 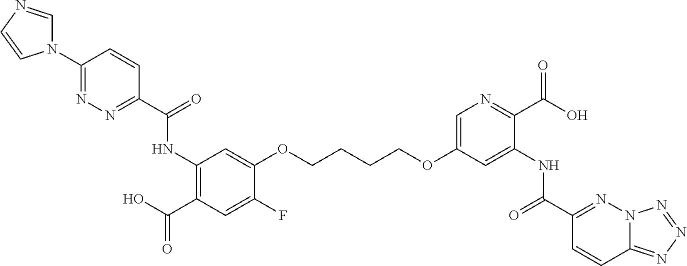 [SMILES: O=C(Nc1cc(OCCCCOc2cnc(C(=O)O)c(NC(=O)c3ccc4nnnn4n3)c2)c(F)cc1C(=O)O)c1ccc(-n2ccnc2)nn1] | + | ++ |
| 84 | 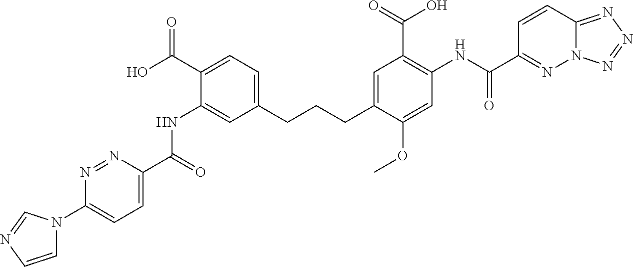 [SMILES: COc1cc(NC(=O)c2ccc3nnnn3n2)c(C(=O)O)cc1CCCc1ccc(C(=O)O)c(NC(=O)c2ccc(-n3ccnc3)nn2)c1] | ++ | ++ |
| 85 | 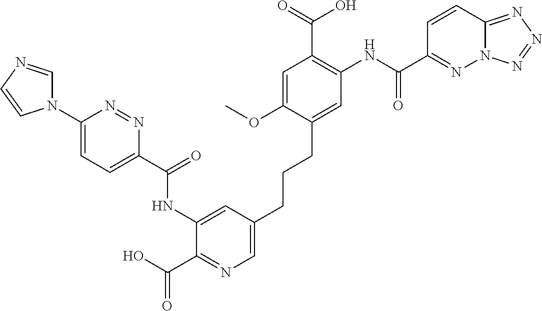 [SMILES: COc1cc(C(=O)O)c(NC(=O)c2ccc3nnnn3n2)cc1CCCc1cnc(C(=O)O)c(NC(=O)c2ccc(-n3ccnc3)nn2)c1] | + | ++ |
| 86 | 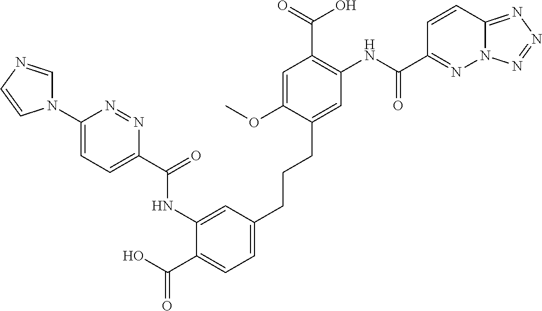 [SMILES: COc1cc(C(=O)O)c(NC(=O)c2ccc3nnnn3n2)cc1CCCc1ccc(C(=O)O)c(NC(=O)c2ccc(-n3ccnc3)nn2)c1] | +++ | +++ |
| 87 | 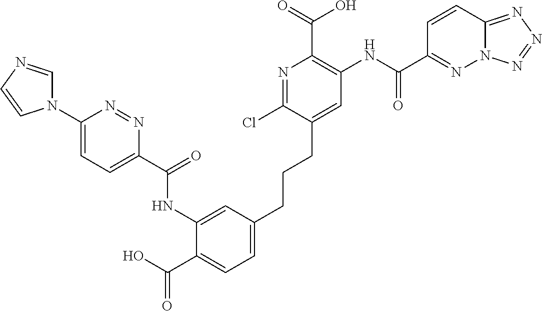 [SMILES: O=C(Nc1cc(CCCc2cc(NC(=O)c3ccc4nnnn4n3)c(C(=O)O)nc2Cl)ccc1C(=O)O)c1ccc(-n2ccnc2)nn1] | +++ | +++ |
| 88 | 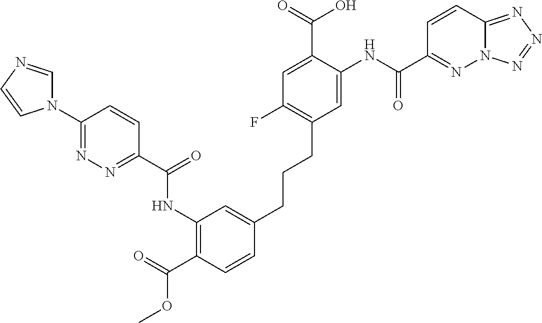 [SMILES: COC(=O)c1ccc(CCCc2cc(NC(=O)c3ccc4nnnn4n3)c(C(=O)O)cc2F)cc1NC(=O)c1ccc(-n2ccnc2)nn1] | ++ | ++ |
| 89 | 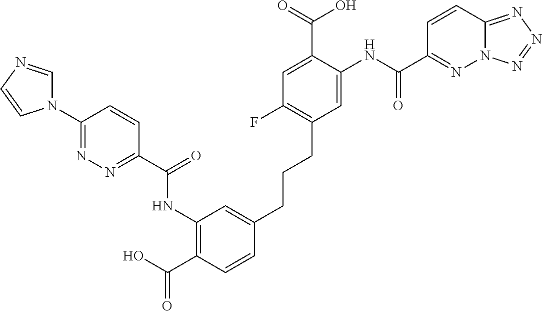 [SMILES: O=C(Nc1cc(CCCc2cc(NC(=O)c3ccc4nnnn4n3)c(C(=O)O)cc2F)ccc1C(=O)O)c1ccc(-n2ccnc2)nn1] | +++ | +++ |
| 90 | 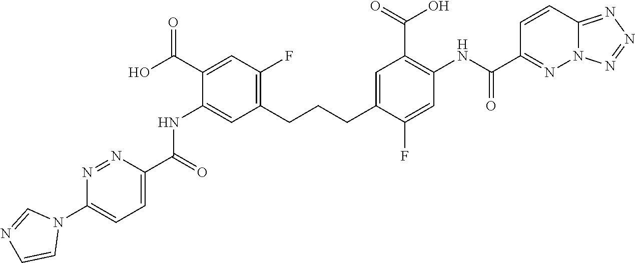 [SMILES: O=C(Nc1cc(CCCc2cc(C(=O)O)c(NC(=O)c3ccc4nnnn4n3)cc2F)c(F)cc1C(=O)O)c1ccc(-n2ccnc2)nn1] | ++ | ++++ |
| 91 | 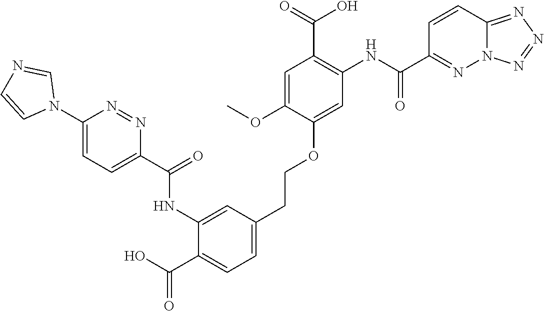 [SMILES: COc1cc(C(=O)O)c(NC(=O)c2ccc3nnnn3n2)cc1OCCc1ccc(C(=O)O)c(NC(=O)c2ccc(-n3ccnc3)nn2)c1] | + | ++ |
| 92 | 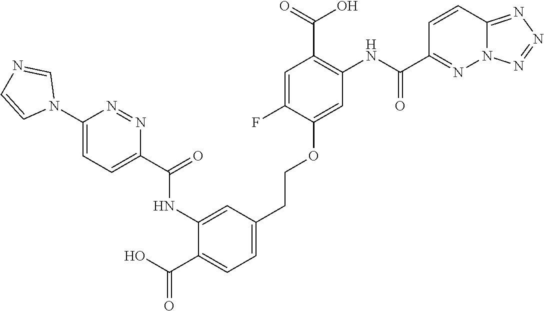 [SMILES: O=C(Nc1cc(CCOc2cc(NC(=O)c3ccc4nnnn4n3)c(C(=O)O)cc2F)ccc1C(=O)O)c1ccc(-n2ccnc2)nn1] | +++ | +++ |
| 93 | 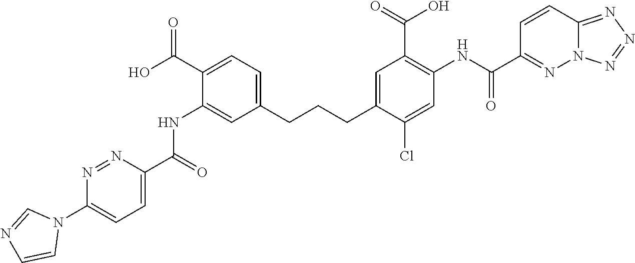 [SMILES: O=C(Nc1cc(CCCc2cc(C(=O)O)c(NC(=O)c3ccc4nnnn4n3)cc2Cl)ccc1C(=O)O)c1ccc(-n2ccnc2)nn1] | +++ | +++ |
| 94 | 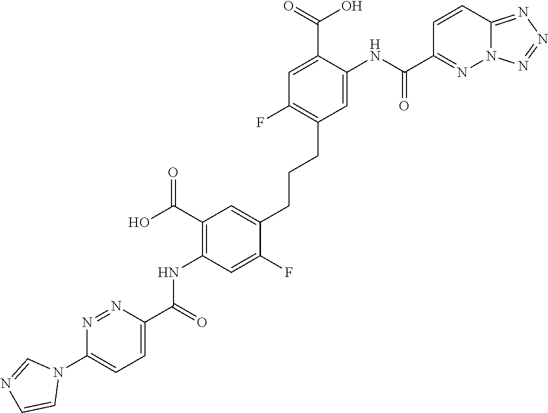 [SMILES: O=C(Nc1cc(F)c(CCCc2cc(NC(=O)c3ccc4nnnn4n3)c(C(=O)O)cc2F)cc1C(=O)O)c1ccc(-n2ccnc2)nn1] | +++ | ++++ |
| 95 | 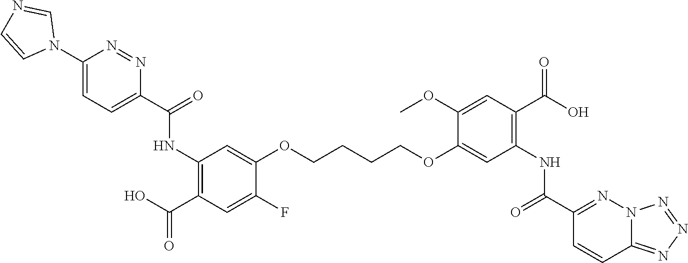 [SMILES: COc1cc(C(=O)O)c(NC(=O)c2ccc3nnnn3n2)cc1OCCCCOc1cc(NC(=O)c2ccc(-n3ccnc3)nn2)c(C(=O)O)cc1F] | ++ | +++ |
| 96 | 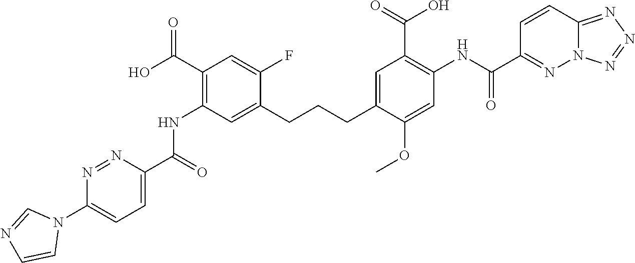 [SMILES: COc1cc(NC(=O)c2ccc3nnnn3n2)c(C(=O)O)cc1CCCc1cc(NC(=O)c2ccc(-n3ccnc3)nn2)c(C(=O)O)cc1F] | +++ | +++ |
| 97 | 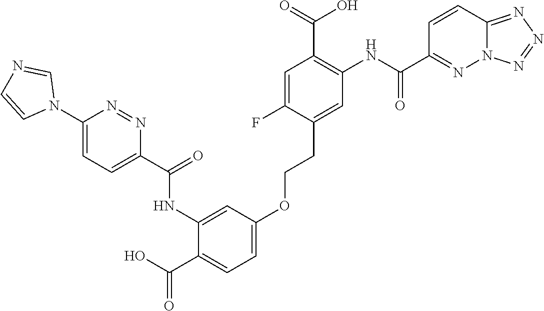 [SMILES: O=C(Nc1cc(OCCc2cc(NC(=O)c3ccc4nnnn4n3)c(C(=O)O)cc2F)ccc1C(=O)O)c1ccc(-n2ccnc2)nn1] | ++ | ++ |
| 98 | 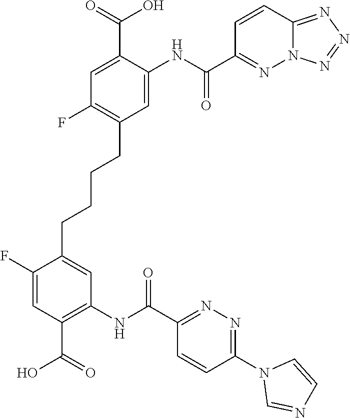 [SMILES: O=C(Nc1cc(CCCCc2cc(NC(=O)c3ccc4nnnn4n3)c(C(=O)O)cc2F)c(F)cc1C(=O)O)c1ccc(-n2ccnc2)nn1] | +++ | +++ |
| 99 | 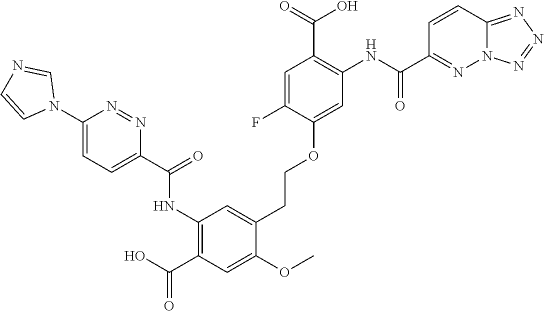 [SMILES: COc1cc(C(=O)O)c(NC(=O)c2ccc(-n3ccnc3)nn2)cc1CCOc1cc(NC(=O)c2ccc3nnnn3n2)c(C(=O)O)cc1F] | ++ | ++ |
| 100 | 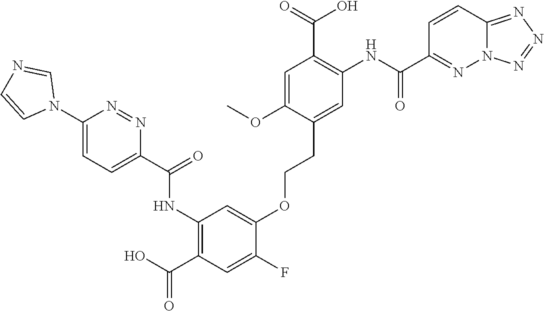 [SMILES: COc1cc(C(=O)O)c(NC(=O)c2ccc3nnnn3n2)cc1CCOc1cc(NC(=O)c2ccc(-n3ccnc3)nn2)c(C(=O)O)cc1F] | +++ | ++ |
| 101 | 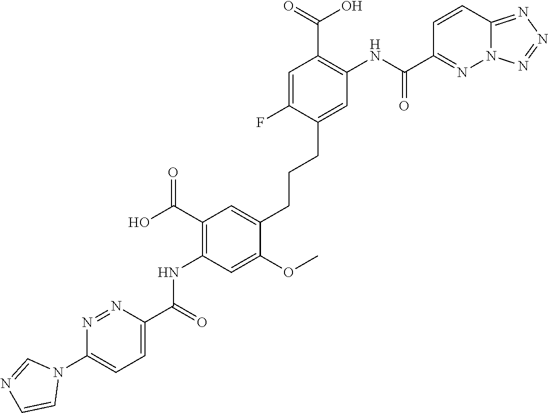 [SMILES: COc1cc(NC(=O)c2ccc(-n3ccnc3)nn2)c(C(=O)O)cc1CCCc1cc(NC(=O)c2ccc3nnnn3n2)c(C(=O)O)cc1F] | +++ | ++ |
| 102 | 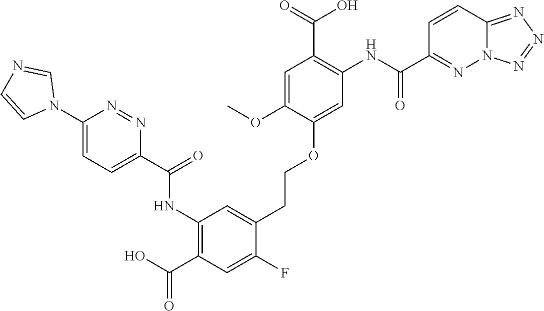 [SMILES: COc1cc(C(=O)O)c(NC(=O)c2ccc3nnnn3n2)cc1OCCc1cc(NC(=O)c2ccc(-n3ccnc3)nn2)c(C(=O)O)cc1F] | ++ | ++ |
| 103 | 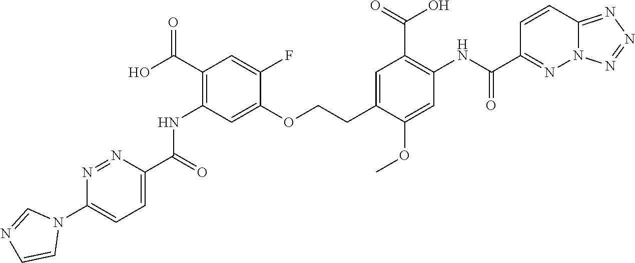 [SMILES: COc1cc(NC(=O)c2ccc3nnnn3n2)c(C(=O)O)cc1CCOc1cc(NC(=O)c2ccc(-n3ccnc3)nn2)c(C(=O)O)cc1F] | ++ | ++ |
| 104 | 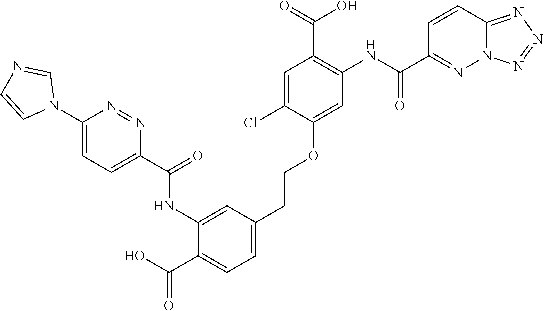 [SMILES: O=C(Nc1cc(CCOc2cc(NC(=O)c3ccc4nnnn4n3)c(C(=O)O)cc2Cl)ccc1C(=O)O)c1ccc(-n2ccnc2)nn1] | +++ | +++ |
| 105 | 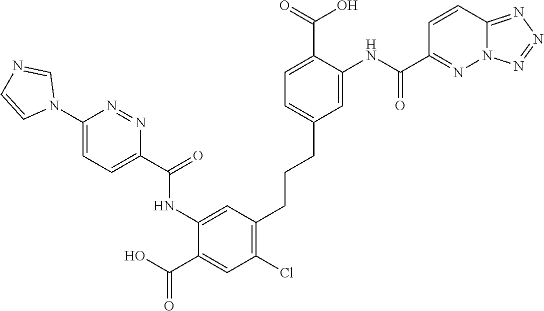 [SMILES: O=C(Nc1cc(CCCc2ccc(C(=O)O)c(NC(=O)c3ccc4nnnn4n3)c2)c(Cl)cc1C(=O)O)c1ccc(-n2ccnc2)nn1] | ++ | +++ |
| 106 | 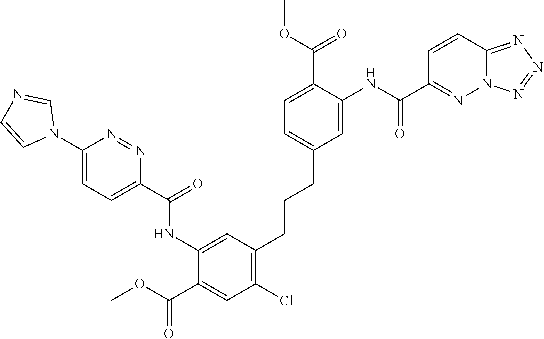 [SMILES: COC(=O)c1cc(Cl)c(CCCc2ccc(C(=O)OC)c(NC(=O)c3ccc4nnnn4n3)c2)cc1NC(=O)c1ccc(-n2ccnc2)nn1] | ++++ | |
| 107 | 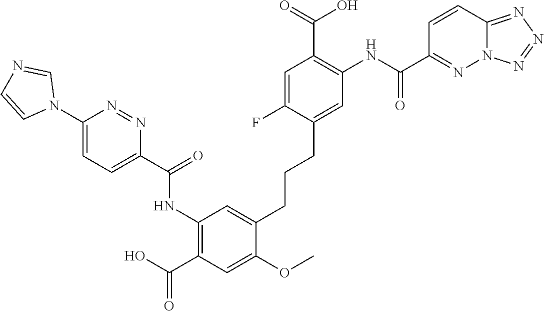 [SMILES: COc1cc(C(=O)O)c(NC(=O)c2ccc(-n3ccnc3)nn2)cc1CCCc1cc(NC(=O)c2ccc3nnnn3n2)c(C(=O)O)cc1F] | +++ | +++ |
| 108 | 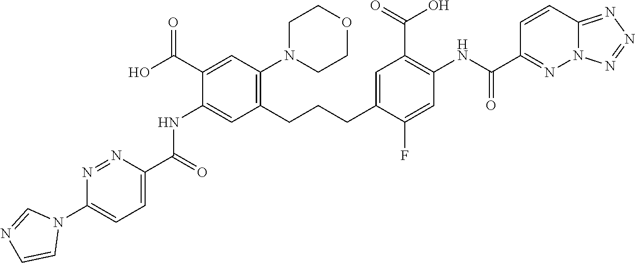 [SMILES: O=C(Nc1cc(CCCc2cc(C(=O)O)c(NC(=O)c3ccc4nnnn4n3)cc2F)c(N2CCOCC2)cc1C(=O)O)c1ccc(-n2ccnc2)nn1] | − | + |
| 109 | 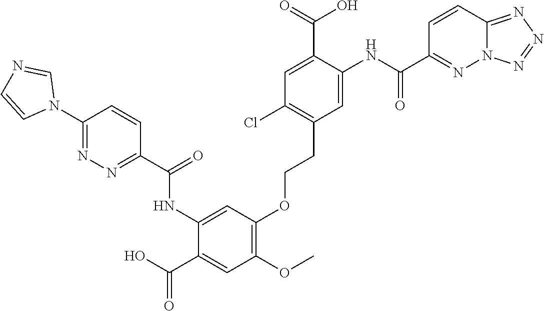 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1Cl] | ++ | ++ |
| 110 | 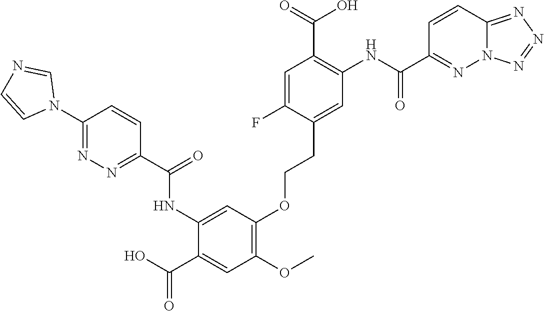 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1F] | + | ++ |
| 111 | 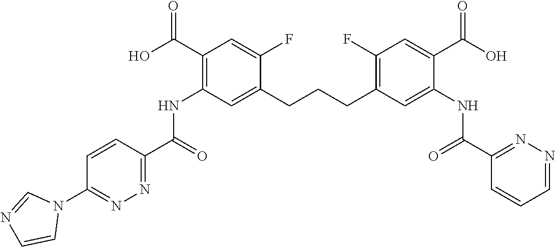 [SMILES: O=C(Nc1cc(CCCc2cc(NC(=O)c3ccc(-n4ccnc4)nn3)c(C(=O)O)cc2F)c(F)cc1C(=O)O)c1cccnn1] | +++ | +++ |
| 112 | 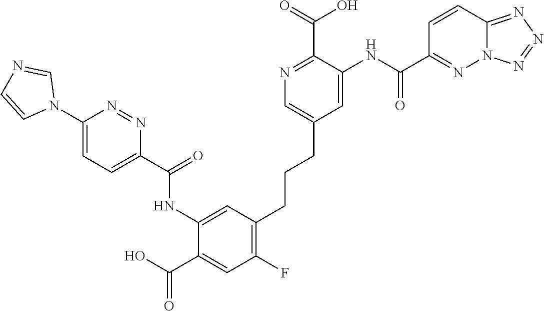 [SMILES: O=C(Nc1cc(CCCc2cnc(C(=O)O)c(NC(=O)c3ccc4nnnn4n3)c2)c(F)cc1C(=O)O)c1ccc(-n2ccnc2)nn1] | ++ | +++ |
| 113 | 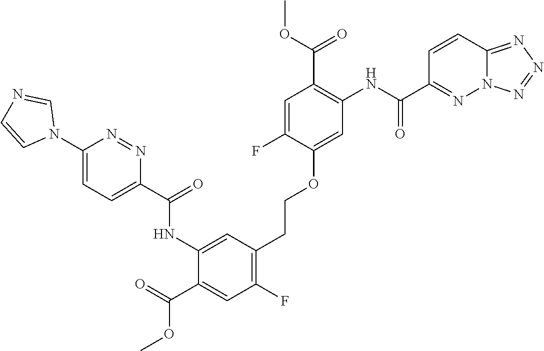 [SMILES: COC(=O)c1cc(F)c(CCOc2cc(NC(=O)c3ccc4nnnn4n3)c(C(=O)OC)cc2F)cc1NC(=O)c1ccc(-n2ccnc2)nn1] | ++++ | |
| 114 | 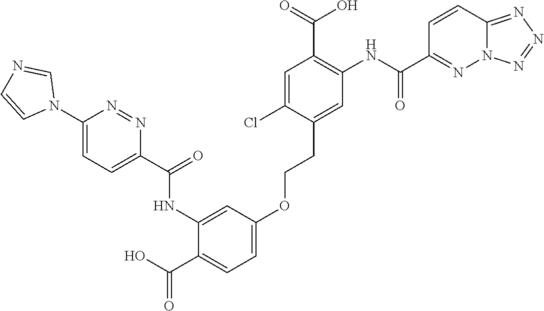 [SMILES: O=C(Nc1cc(OCCc2cc(NC(=O)c3ccc4nnnn4n3)c(C(=O)O)cc2Cl)ccc1C(=O)O)c1ccc(-n2ccnc2)nn1] | +++ | ++ |
| 115 | 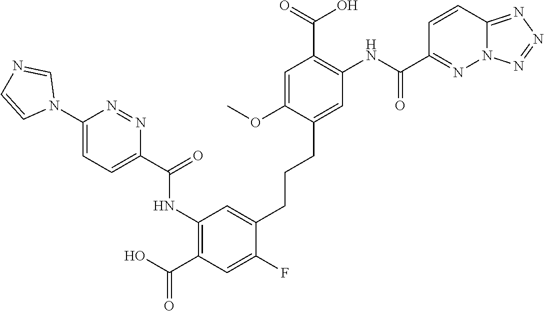 [SMILES: COc1cc(C(=O)O)c(NC(=O)c2ccc3nnnn3n2)cc1CCCc1cc(NC(=O)c2ccc(-n3ccnc3)nn2)c(C(=O)O)cc1F] | +++ | +++ |
| 116 | 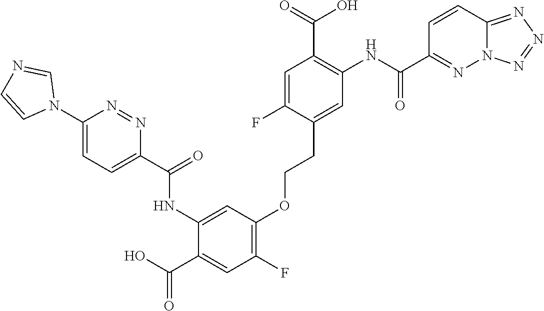 [SMILES: O=C(Nc1cc(OCCc2cc(NC(=O)c3ccc4nnnn4n3)c(C(=O)O)cc2F)c(F)cc1C(=O)O)c1ccc(-n2ccnc2)nn1] | +++ | +++ |
| 117 | 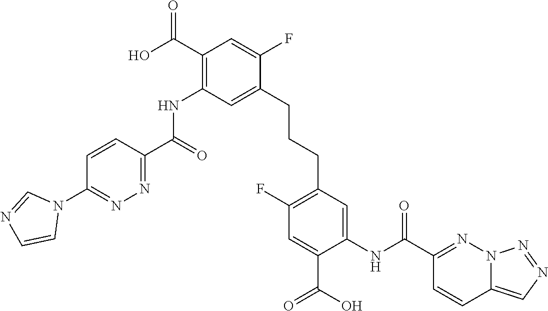 [SMILES: O=C(Nc1cc(CCCc2cc(NC(=O)c3ccc4cnnn4n3)c(C(=O)O)cc2F)c(F)cc1C(=O)O)c1ccc(-n2ccnc2)nn1] | ++ | +++ |
| 118 | 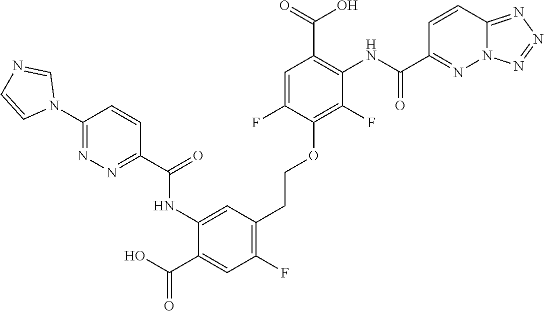 [SMILES: O=C(Nc1cc(CCOc2c(F)cc(C(=O)O)c(NC(=O)c3ccc4nnnn4n3)c2F)c(F)cc1C(=O)O)c1ccc(-n2ccnc2)nn1] | ++ | ++ |
| 119 | 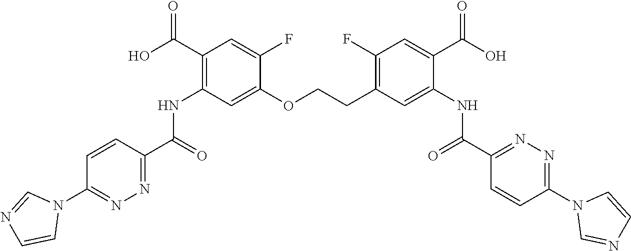 [SMILES: O=C(Nc1cc(CCOc2cc(NC(=O)c3ccc(-n4ccnc4)nn3)c(C(=O)O)cc2F)c(F)cc1C(=O)O)c1ccc(-n2ccnc2)nn1] | +++ | +++ |
| 120 | 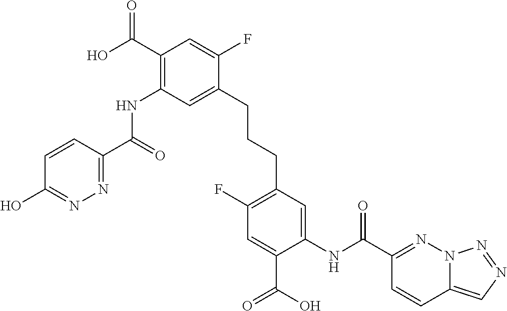 [SMILES: O=C(Nc1cc(CCCc2cc(NC(=O)c3ccc4cnnn4n3)c(C(=O)O)cc2F)c(F)cc1C(=O)O)c1ccc(O)nn1] | − | ++ |
| 121 | 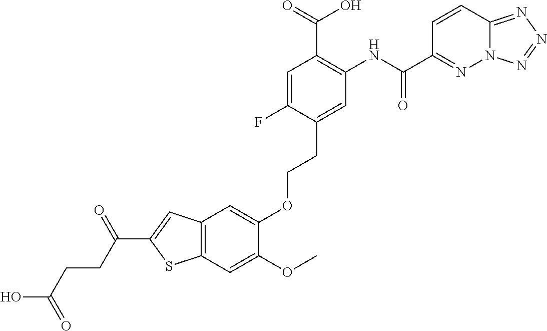 [SMILES: COc1cc2sc(C(=O)CCC(=O)O)cc2cc1OCCc1cc(NC(=O)c2ccc3nnnn3n2)c(C(=O)O)cc1F] | − | − |
| 122 | 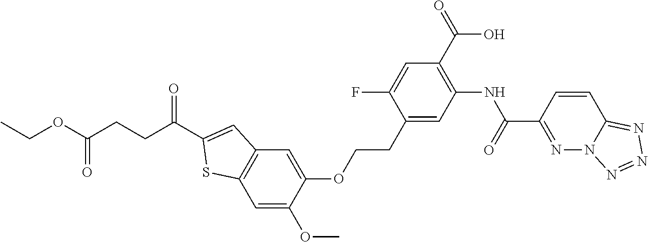 [SMILES: CCOC(=O)CCC(=O)c1cc2cc(OCCc3cc(NC(=O)c4ccc5nnnn5n4)c(C(=O)O)cc3F)c(OC)cc2s1] | − | ++ |
| 123 | 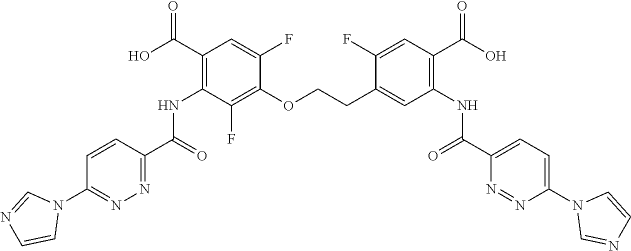 [SMILES: O=C(Nc1cc(CCOc2c(F)cc(C(=O)O)c(NC(=O)c3ccc(-n4ccnc4)nn3)c2F)c(F)cc1C(=O)O)c1ccc(-n2ccnc2)nn1] | +++ | ++ |
| 124 | 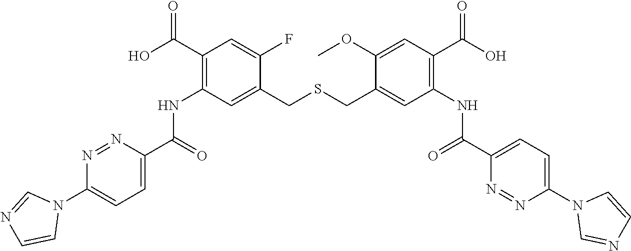 [SMILES: COc1cc(C(=O)O)c(NC(=O)c2ccc(-n3ccnc3)nn2)cc1CSCc1cc(NC(=O)c2ccc(-n3ccnc3)nn2)c(C(=O)O)cc1F] | +++ | +++ |
| 125 | 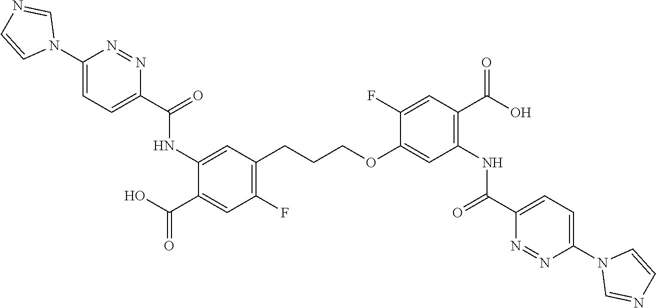 [SMILES: O=C(Nc1cc(CCCOc2cc(NC(=O)c3ccc(-n4ccnc4)nn3)c(C(=O)O)cc2F)c(F)cc1C(=O)O)c1ccc(-n2ccnc2)nn1] | ++ | ++++ |
| 126 | 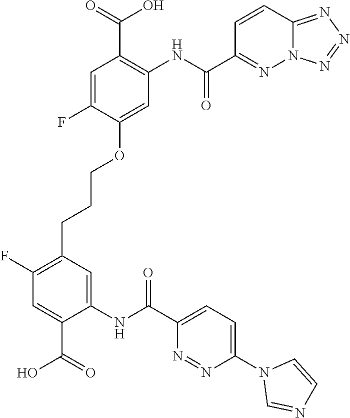 [SMILES: O=C(Nc1cc(CCCOc2cc(NC(=O)c3ccc4nnnn4n3)c(C(=O)O)cc2F)c(F)cc1C(=O)O)c1ccc(-n2ccnc2)nn1] | ++ | ++++ |
| 127 | 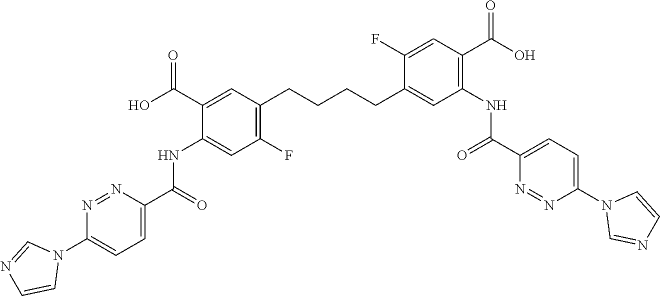 [SMILES: O=C(Nc1cc(CCCCc2cc(C(=O)O)c(NC(=O)c3ccc(-n4ccnc4)nn3)cc2F)c(F)cc1C(=O)O)c1ccc(-n2ccnc2)nn1] | ++ | ++++ |
| 128 | 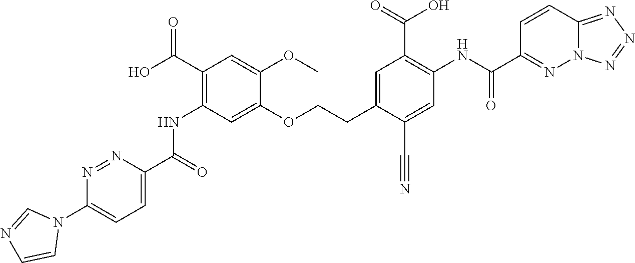 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1C#N] | +++ | +++ |
| 129 | 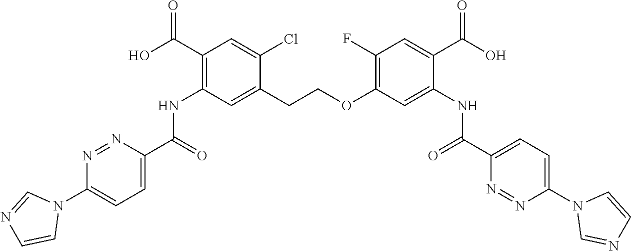 [SMILES: O=C(Nc1cc(OCCc2cc(NC(=O)c3ccc(-n4ccnc4)nn3)c(C(=O)O)cc2Cl)c(F)cc1C(=O)O)c1ccc(-n2ccnc2)nn1] | +++ | +++ |
| 130 | 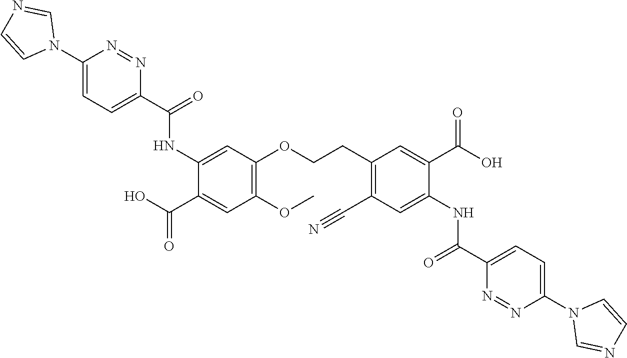 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1C#N] | +++ | ++++ |
| 131 | 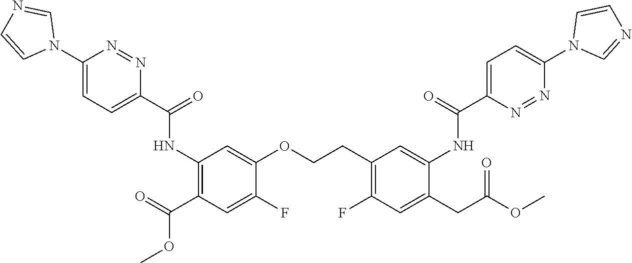 [SMILES: COC(=O)Cc1cc(F)c(CCOc2cc(NC(=O)c3ccc(-n4ccnc4)nn3)c(C(=O)OC)cc2F)cc1NC(=O)c1ccc(-n2ccnc2)nn1] | ++ | |
| 132 | 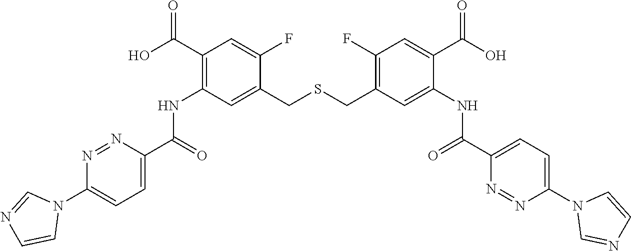 [SMILES: O=C(Nc1cc(CSCc2cc(NC(=O)c3ccc(-n4ccnc4)nn3)c(C(=O)O)cc2F)c(F)cc1C(=O)O)c1ccc(-n2ccnc2)nn1] | ++++ | ++++ |
| 133 | 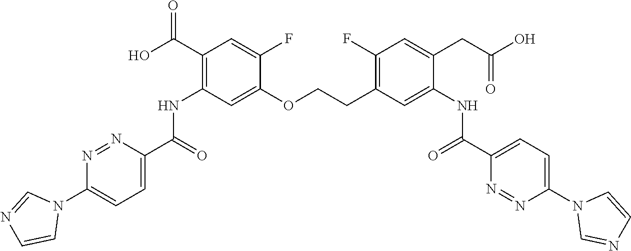 [SMILES: O=C(O)Cc1cc(F)c(CCOc2cc(NC(=O)c3ccc(-n4ccnc4)nn3)c(C(=O)O)cc2F)cc1NC(=O)c1ccc(-n2ccnc2)nn1] | − | + |
| 134 | 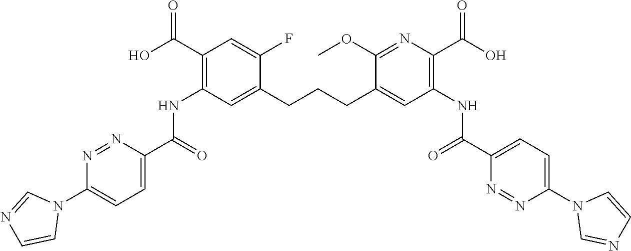 [SMILES: COc1nc(C(=O)O)c(NC(=O)c2ccc(-n3ccnc3)nn2)cc1CCCc1cc(NC(=O)c2ccc(-n3ccnc3)nn2)c(C(=O)O)cc1F] | +++ | ++++ |
| 135 | 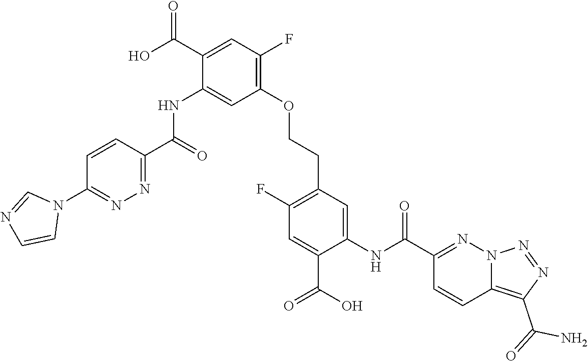 [SMILES: NC(=O)c1nnn2nc(C(=O)Nc3cc(CCOc4cc(NC(=O)c5ccc(-n6ccnc6)nn5)c(C(=O)O)cc4F)c(F)cc3C(=O)O)ccc12] | +++ | +++ |
| 136 | 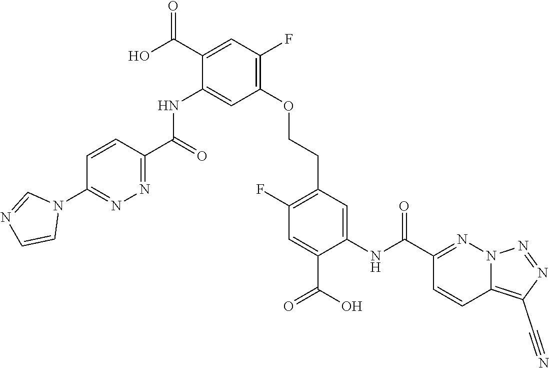 [SMILES: N#Cc1nnn2nc(C(=O)Nc3cc(CCOc4cc(NC(=O)c5ccc(-n6ccnc6)nn5)c(C(=O)O)cc4F)c(F)cc3C(=O)O)ccc12] | + | ++ |
| 137 | 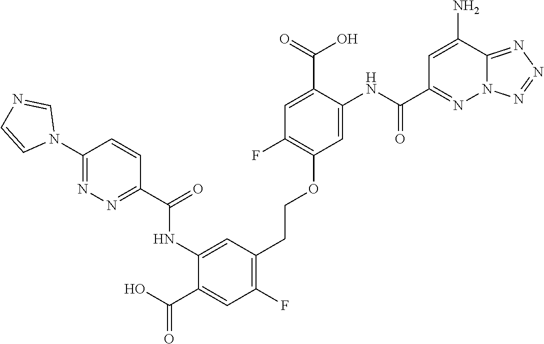 [SMILES: Nc1cc(C(=O)Nc2cc(OCCc3cc(NC(=O)c4ccc(-n5ccnc5)nn4)c(C(=O)O)cc3F)c(F)cc2C(=O)O)nn2nnnc12] | +++ | +++ |
| 138 | 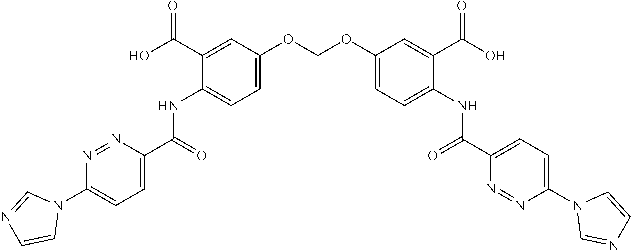 [SMILES: O=C(Nc1ccc(OCOc2ccc(NC(=O)c3ccc(-n4ccnc4)nn3)c(C(=O)O)c2)cc1C(=O)O)c1ccc(-n2ccnc2)nn1] | − | + |
| 139 | 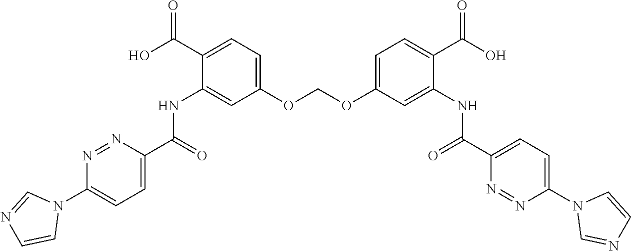 [SMILES: O=C(Nc1cc(OCOc2ccc(C(=O)O)c(NC(=O)c3ccc(-n4ccnc4)nn3)c2)ccc1C(=O)O)c1ccc(-n2ccnc2)nn1] | − | − |
| 140 | 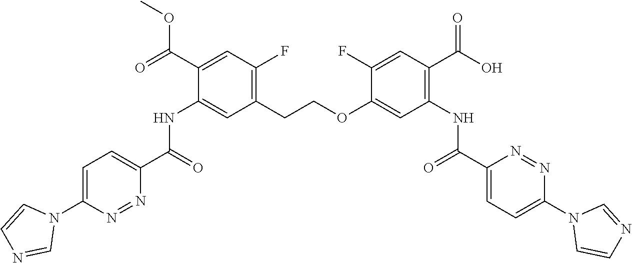 [SMILES: COC(=O)c1cc(F)c(CCOc2cc(NC(=O)c3ccc(-n4ccnc4)nn3)c(C(=O)O)cc2F)cc1NC(=O)c1ccc(-n2ccnc2)nn1] | ++++ | ++ |
| 141 | 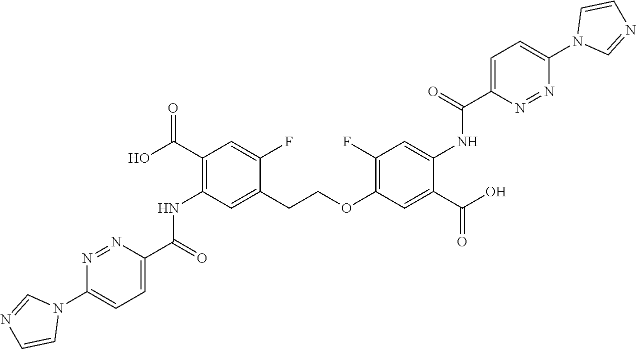 [SMILES: O=C(Nc1cc(CCOc2cc(C(=O)O)c(NC(=O)c3ccc(-n4ccnc4)nn3)cc2F)c(F)cc1C(=O)O)c1ccc(-n2ccnc2)nn1] | +++ | ++++ |
| 142 | 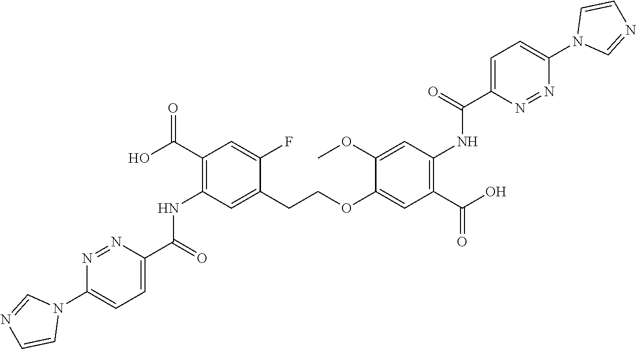 [SMILES: COc1cc(NC(=O)c2ccc(-n3ccnc3)nn2)c(C(=O)O)cc1OCCc1cc(NC(=O)c2ccc(-n3ccnc3)nn2)c(C(=O)O)cc1F] | +++ | +++ |
| 143 | 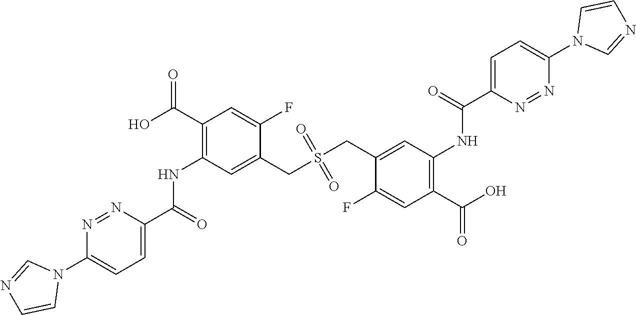 [SMILES: O=C(Nc1cc(CS(=O)(=O)Cc2cc(NC(=O)c3ccc(-n4ccnc4)nn3)c(C(=O)O)cc2F)c(F)cc1C(=O)O)c1ccc(-n2ccnc2)nn1] | + | +++ |
| 144 | 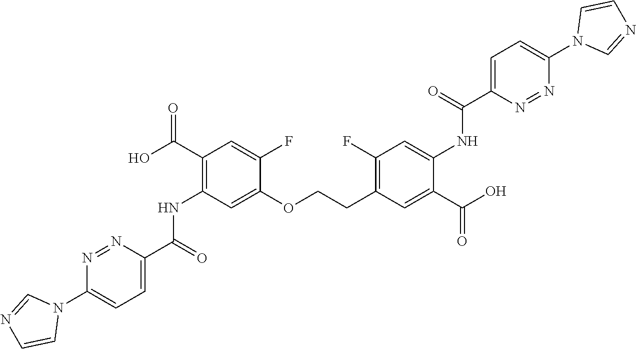 [SMILES: O=C(Nc1cc(OCCc2cc(C(=O)O)c(NC(=O)c3ccc(-n4ccnc4)nn3)cc2F)c(F)cc1C(=O)O)c1ccc(-n2ccnc2)nn1] | +++ | ++++ |
| 145 | 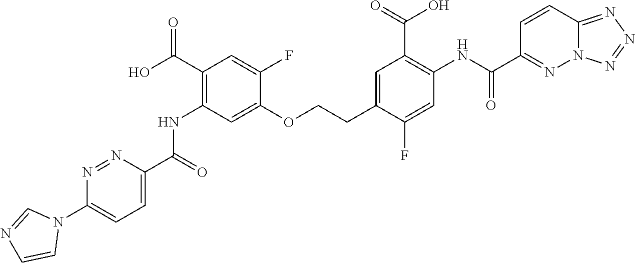 [SMILES: O=C(Nc1cc(OCCc2cc(C(=O)O)c(NC(=O)c3ccc4nnnn4n3)cc2F)c(F)cc1C(=O)O)c1ccc(-n2ccnc2)nn1] | +++ | +++ |
| 146 | 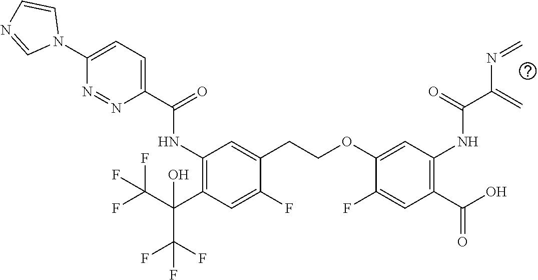 [SMILES: C=NC(=C)C(=O)Nc1cc(OCCc2cc(NC(=O)c3ccc(-n4ccnc4)nn3)c(C(O)(C(F)(F)F)C(F)(F)F)cc2F)c(F)cc1C(=O)O] | − | + |
| 147 | 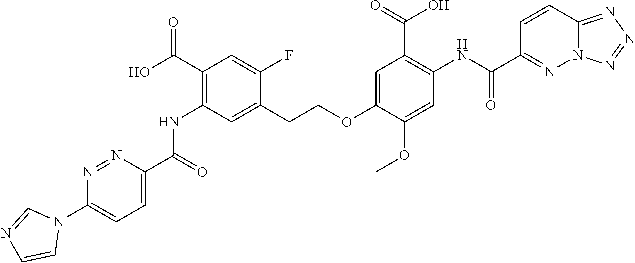 [SMILES: COc1cc(NC(=O)c2ccc3nnnn3n2)c(C(=O)O)cc1OCCc1cc(NC(=O)c2ccc(-n3ccnc3)nn2)c(C(=O)O)cc1F] | + | ++ |
| 148 | 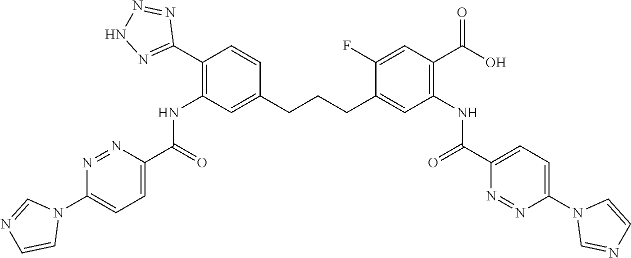 [SMILES: O=C(Nc1cc(CCCc2ccc(-c3nn[nH]n3)c(NC(=O)c3ccc(-n4ccnc4)nn3)c2)c(F)cc1C(=O)O)c1ccc(-n2ccnc2)nn1] | − | + |
| 149 | 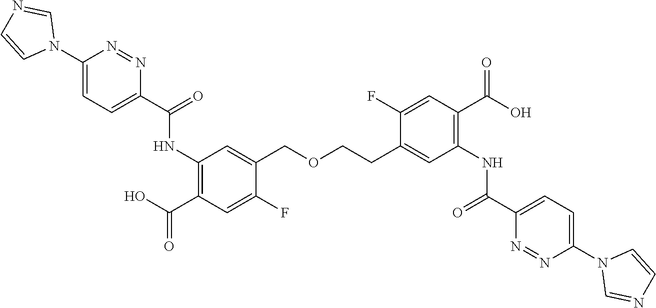 [SMILES: O=C(Nc1cc(CCOCc2cc(NC(=O)c3ccc(-n4ccnc4)nn3)c(C(=O)O)cc2F)c(F)cc1C(=O)O)c1ccc(-n2ccnc2)nn1] | ++ | +++ |
| 150 | 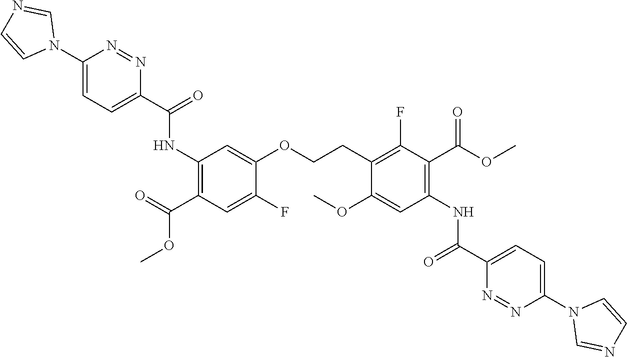 [SMILES: COC(=O)c1cc(F)c(OCCc2c(OC)cc(NC(=O)c3ccc(-n4ccnc4)nn3)c(C(=O)OC)c2F)cc1NC(=O)c1ccc(-n2ccnc2)nn1] | + | |
| 151 | 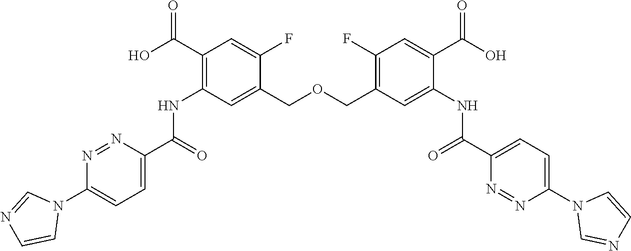 [SMILES: O=C(Nc1cc(COCc2cc(NC(=O)c3ccc(-n4ccnc4)nn3)c(C(=O)O)cc2F)c(F)cc1C(=O)O)c1ccc(-n2ccnc2)nn1] | +++ | +++ |
| 152 | 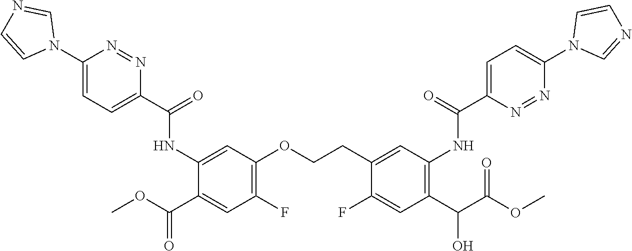 [SMILES: COC(=O)c1cc(F)c(OCCc2cc(NC(=O)c3ccc(-n4ccnc4)nn3)c(C(O)C(=O)OC)cc2F)cc1NC(=O)c1ccc(-n2ccnc2)nn1] | ++ | |
| 153 | 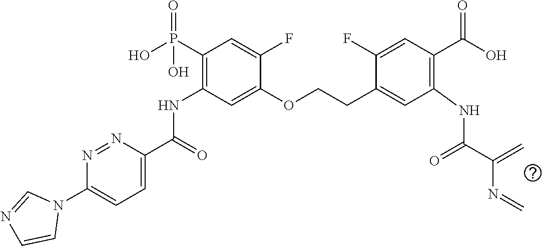 [SMILES: C=NC(=C)C(=O)Nc1cc(CCOc2cc(NC(=O)c3ccc(-n4ccnc4)nn3)c(P(=O)(O)O)cc2F)c(F)cc1C(=O)O] | − | ++ |
| 154 | 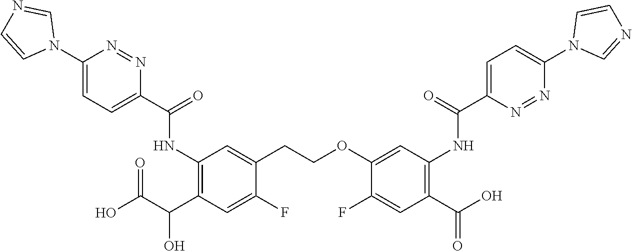 [SMILES: O=C(Nc1cc(OCCc2cc(NC(=O)c3ccc(-n4ccnc4)nn3)c(C(O)C(=O)O)cc2F)c(F)cc1C(=O)O)c1ccc(-n2ccnc2)nn1] | ++ | ++ |
| 155 | 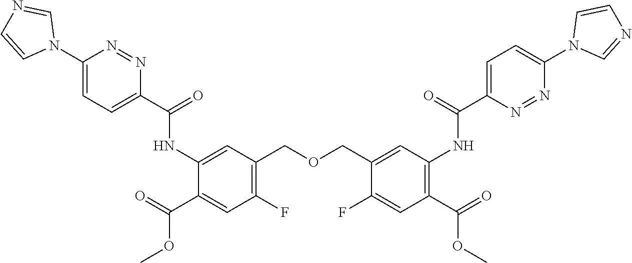 [SMILES: COC(=O)c1cc(F)c(COCc2cc(NC(=O)c3ccc(-n4ccnc4)nn3)c(C(=O)OC)cc2F)cc1NC(=O)c1ccc(-n2ccnc2)nn1] | ++++ | |
| 156 | 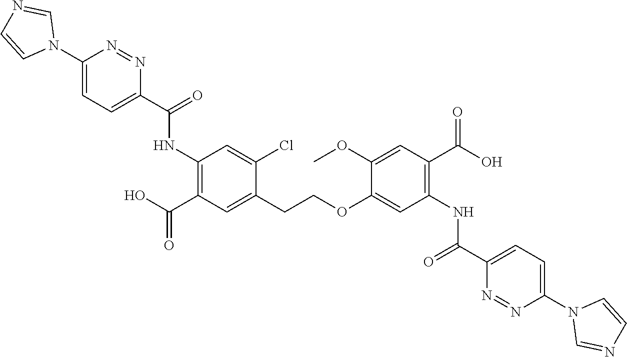 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1Cl] | +++ | ++++ |
| 157 | 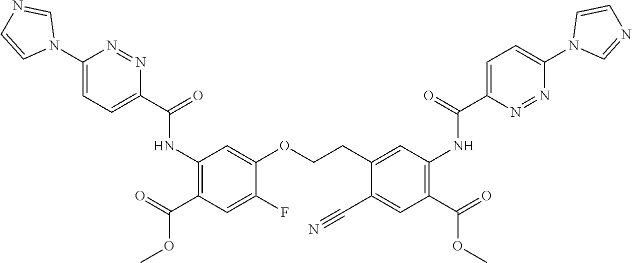 [SMILES: COC(=O)c1cc(F)c(OCCc2cc(NC(=O)c3ccc(-n4ccnc4)nn3)c(C(=O)OC)cc2C#N)cc1NC(=O)c1ccc(-n2ccnc2)nn1] | ++ | |
| 158 | 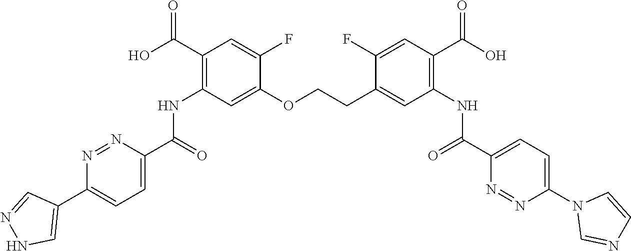 [SMILES: O=C(Nc1cc(OCCc2cc(NC(=O)c3ccc(-n4ccnc4)nn3)c(C(=O)O)cc2F)c(F)cc1C(=O)O)c1ccc(-c2cn[nH]c2)nn1] | − | ++ |
| 159 | 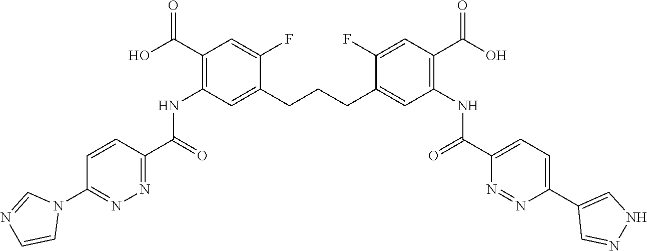 [SMILES: O=C(Nc1cc(CCCc2cc(NC(=O)c3ccc(-n4ccnc4)nn3)c(C(=O)O)cc2F)c(F)cc1C(=O)O)c1ccc(-c2cn[nH]c2)nn1] | +++ | ++++ |
| 160 | 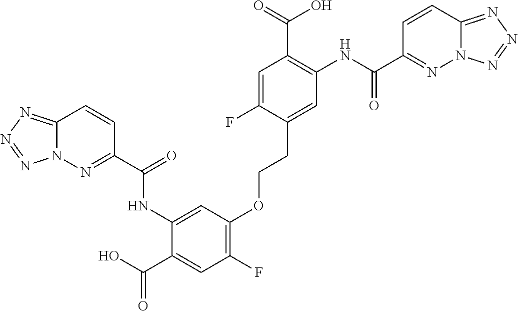 [SMILES: O=C(Nc1cc(CCOc2cc(NC(=O)c3ccc4nnnn4n3)c(C(=O)O)cc2F)c(F)cc1C(=O)O)c1ccc2nnnn2n1] | + | ++ |
| 161 | 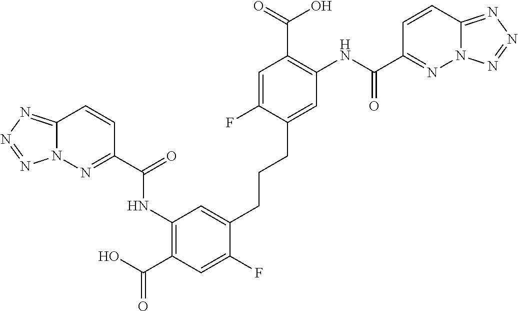 [SMILES: O=C(Nc1cc(CCCc2cc(NC(=O)c3ccc4nnnn4n3)c(C(=O)O)cc2F)c(F)cc1C(=O)O)c1ccc2nnnn2n1] | + | ++ |
| 162 | 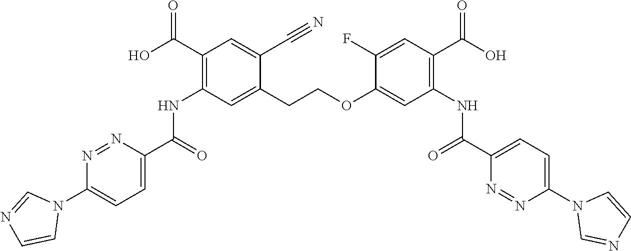 [SMILES: N#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] | +++ | ++++ |
| 163 | 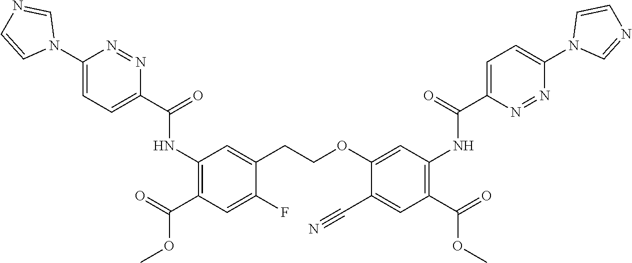 [SMILES: COC(=O)c1cc(F)c(CCOc2cc(NC(=O)c3ccc(-n4ccnc4)nn3)c(C(=O)OC)cc2C#N)cc1NC(=O)c1ccc(-n2ccnc2)nn1] | ++ | |
| 164 | 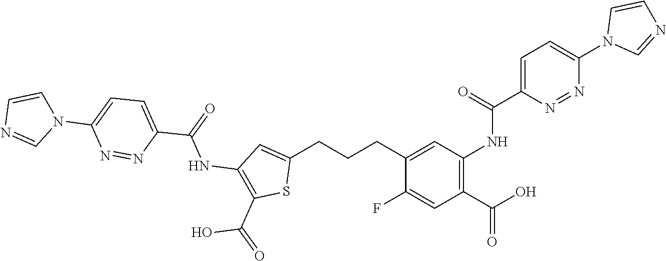 [SMILES: O=C(Nc1cc(CCCc2cc(NC(=O)c3ccc(-n4ccnc4)nn3)c(C(=O)O)s2)c(F)cc1C(=O)O)c1ccc(-n2ccnc2)nn1] | +++ | ++++ |
| 165 | 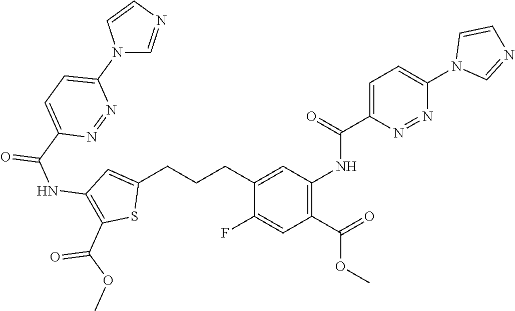 | +++ | |
| 166 | 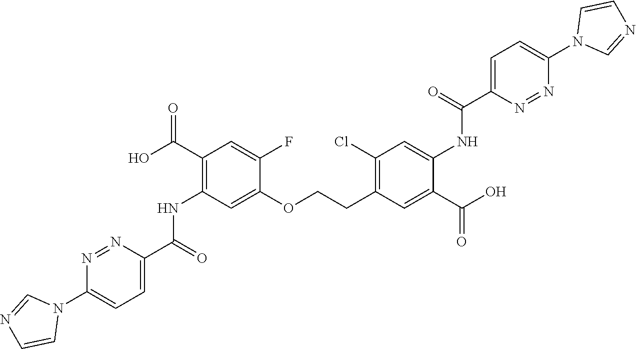 [SMILES: O=C(Nc1cc(OCCc2cc(C(=O)O)c(NC(=O)c3ccc(-n4ccnc4)nn3)cc2Cl)c(F)cc1C(=O)O)c1ccc(-n2ccnc2)nn1] | +++ | ++++ |
| 167 | 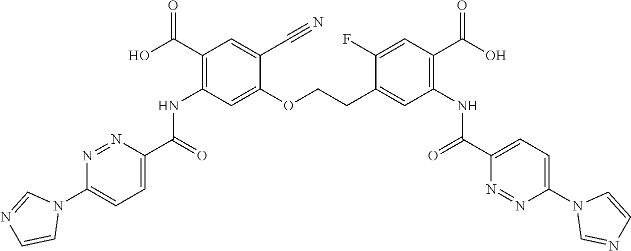 [SMILES: N#Cc1cc(C(=O)O)c(NC(=O)c2ccc(-n3ccnc3)nn2)cc1OCCc1cc(NC(=O)c2ccc(-n3ccnc3)nn2)c(C(=O)O)cc1F] | +++ | +++ |
| 168 | 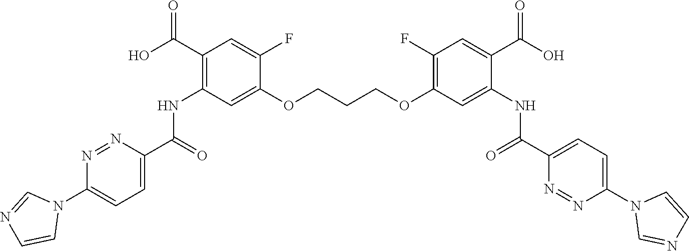 [SMILES: O=C(Nc1cc(OCCCOc2cc(NC(=O)c3ccc(-n4ccnc4)nn3)c(C(=O)O)cc2F)c(F)cc1C(=O)O)c1ccc(-n2ccnc2)nn1] | +++ | ++++ |
| 169 | 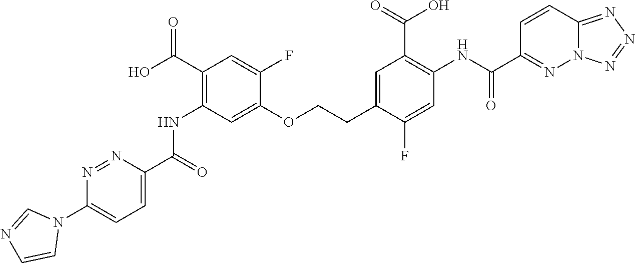 [SMILES: O=C(Nc1cc(OCCc2cc(C(=O)O)c(NC(=O)c3ccc4nnnn4n3)cc2F)c(F)cc1C(=O)O)c1ccc(-n2ccnc2)nn1] | +++ | ++++ |
| 170 | 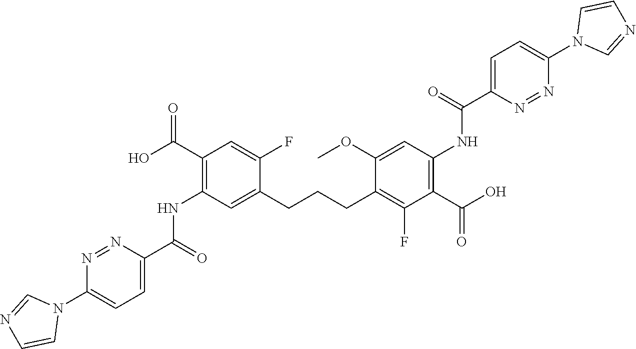 [SMILES: COc1cc(NC(=O)c2ccc(-n3ccnc3)nn2)c(C(=O)O)c(F)c1CCCc1cc(NC(=O)c2ccc(-n3ccnc3)nn2)c(C(=O)O)cc1F] | +++ | +++ |
| 171 | 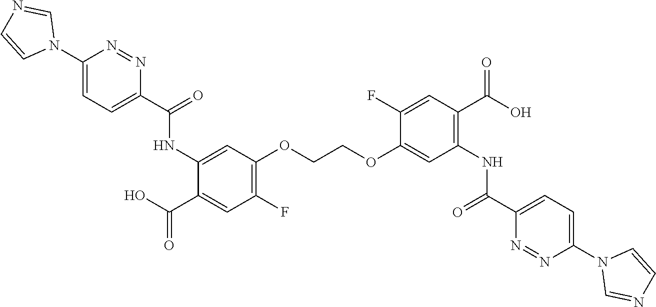 [SMILES: O=C(Nc1cc(OCCOc2cc(NC(=O)c3ccc(-n4ccnc4)nn3)c(C(=O)O)cc2F)c(F)cc1C(=O)O)c1ccc(-n2ccnc2)nn1] | +++ | +++ |
| 172 | 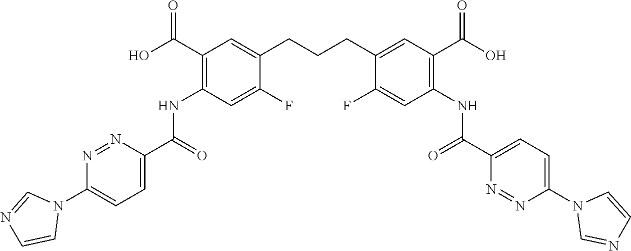 [SMILES: O=C(Nc1cc(F)c(CCCc2cc(C(=O)O)c(NC(=O)c3ccc(-n4ccnc4)nn3)cc2F)cc1C(=O)O)c1ccc(-n2ccnc2)nn1] | + | ++ |
| 173 | 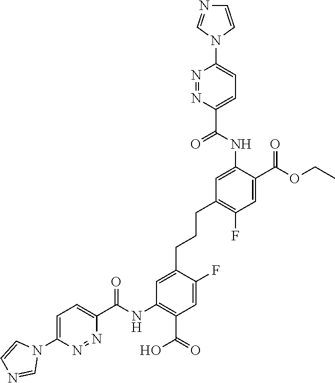 [SMILES: CCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] | ++++ | +++ |
| 174 | 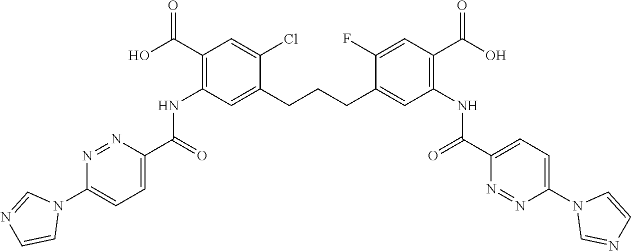 [SMILES: O=C(Nc1cc(CCCc2cc(NC(=O)c3ccc(-n4ccnc4)nn3)c(C(=O)O)cc2Cl)c(F)cc1C(=O)O)c1ccc(-n2ccnc2)nn1] | +++ | ++++ |
| 175 | 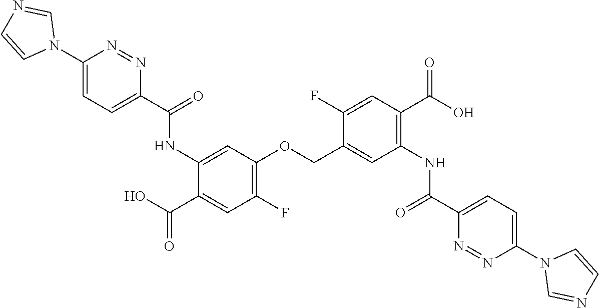 [SMILES: O=C(Nc1cc(COc2cc(NC(=O)c3ccc(-n4ccnc4)nn3)c(C(=O)O)cc2F)c(F)cc1C(=O)O)c1ccc(-n2ccnc2)nn1] | ++ | − |
| 176 | 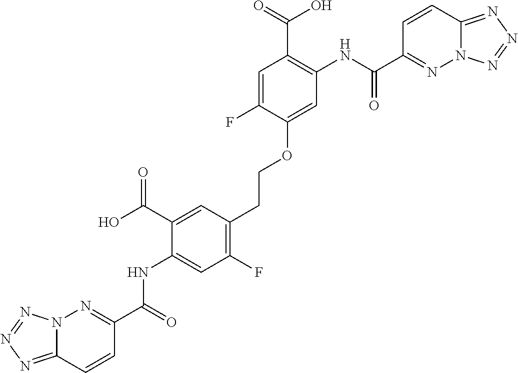 [SMILES: O=C(Nc1cc(OCCc2cc(C(=O)O)c(NC(=O)c3ccc4nnnn4n3)cc2F)c(F)cc1C(=O)O)c1ccc2nnnn2n1] | + | ++ |
| 177 | 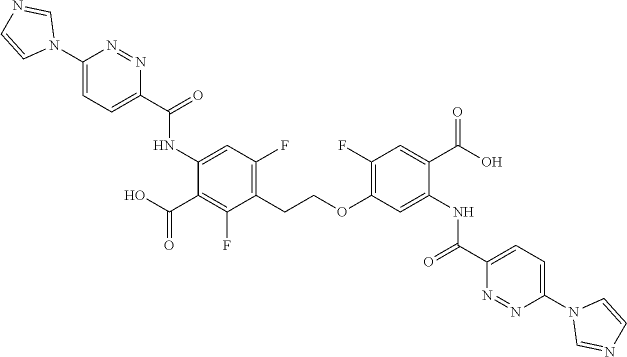 [SMILES: O=C(Nc1cc(OCCc2c(F)cc(NC(=O)c3ccc(-n4ccnc4)nn3)c(C(=O)O)c2F)c(F)cc1C(=O)O)c1ccc(-n2ccnc2)nn1] | ++++ | ++++ |
| 178 | 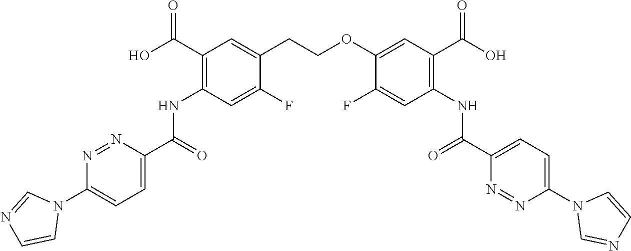 [SMILES: O=C(Nc1cc(F)c(CCOc2cc(C(=O)O)c(NC(=O)c3ccc(-n4ccnc4)nn3)cc2F)cc1C(=O)O)c1ccc(-n2ccnc2)nn1] | − | ++ |
| 179 | 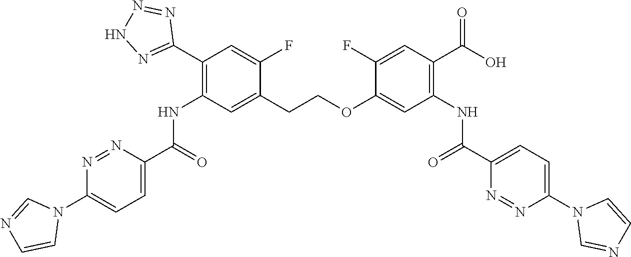 [SMILES: O=C(Nc1cc(OCCc2cc(NC(=O)c3ccc(-n4ccnc4)nn3)c(-c3nn[nH]n3)cc2F)c(F)cc1C(=O)O)c1ccc(-n2ccnc2)nn1] | − | ++ |
| 180 | 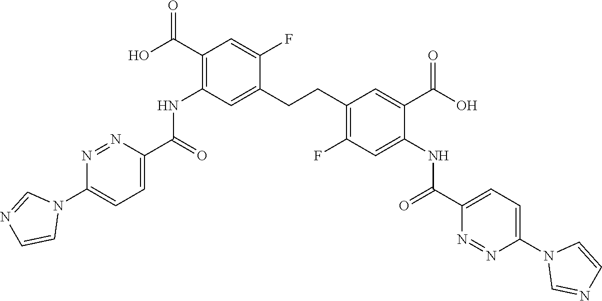 [SMILES: O=C(Nc1cc(CCc2cc(C(=O)O)c(NC(=O)c3ccc(-n4ccnc4)nn3)cc2F)c(F)cc1C(=O)O)c1ccc(-n2ccnc2)nn1] | + | + |
| 181 | 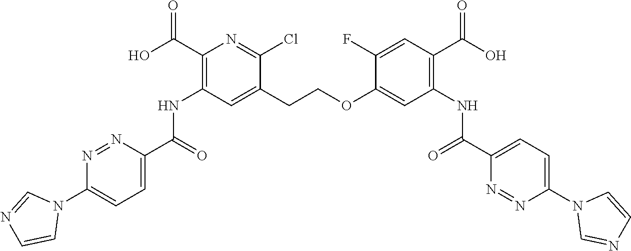 [SMILES: O=C(Nc1cc(OCCc2cc(NC(=O)c3ccc(-n4ccnc4)nn3)c(C(=O)O)nc2Cl)c(F)cc1C(=O)O)c1ccc(-n2ccnc2)nn1] | +++ | +++ |
| 182 | 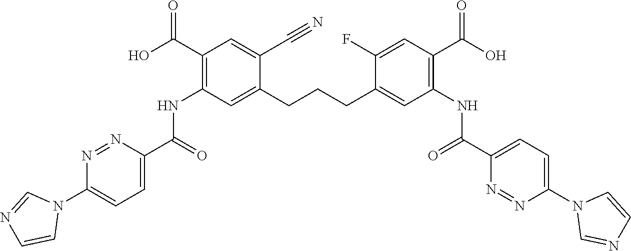 [SMILES: N#Cc1cc(C(=O)O)c(NC(=O)c2ccc(-n3ccnc3)nn2)cc1CCCc1cc(NC(=O)c2ccc(-n3ccnc3)nn2)c(C(=O)O)cc1F] | +++ | ++++ |
| 183 | 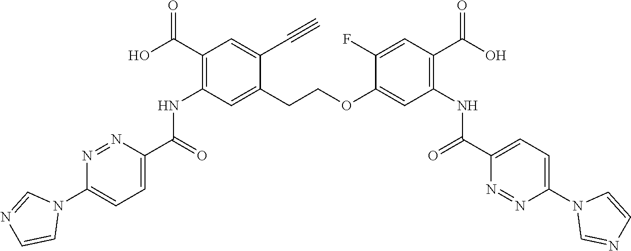 [SMILES: C#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] | ++ | ++ |
| 184 | 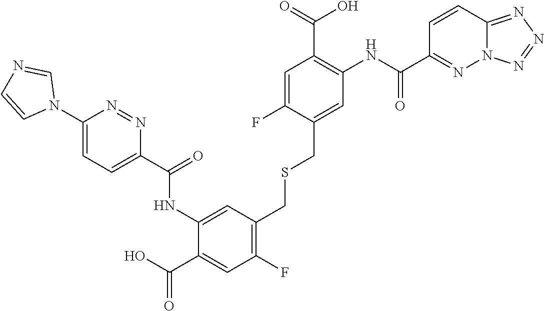 [SMILES: O=C(Nc1cc(CSCc2cc(NC(=O)c3ccc4nnnn4n3)c(C(=O)O)cc2F)c(F)cc1C(=O)O)c1ccc(-n2ccnc2)nn1] | ++ | +++ |
| 185 | 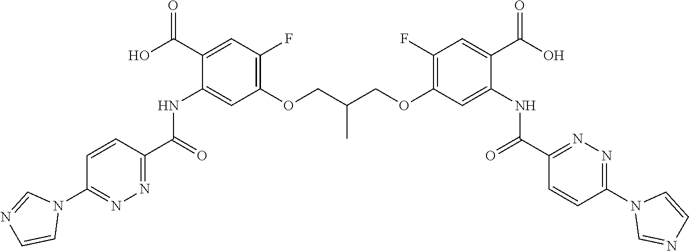 [SMILES: CC(COc1cc(NC(=O)c2ccc(-n3ccnc3)nn2)c(C(=O)O)cc1F)COc1cc(NC(=O)c2ccc(-n3ccnc3)nn2)c(C(=O)O)cc1F] | ++ | +++ |
| 186 | 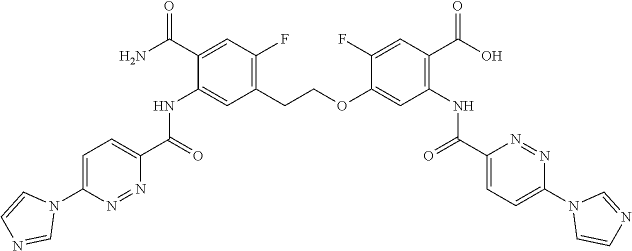 [SMILES: NC(=O)c1cc(F)c(CCOc2cc(NC(=O)c3ccc(-n4ccnc4)nn3)c(C(=O)O)cc2F)cc1NC(=O)c1ccc(-n2ccnc2)nn1] | − | + |
| 187 | 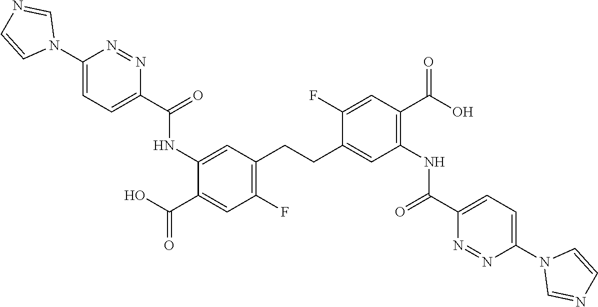 [SMILES: O=C(Nc1cc(CCc2cc(NC(=O)c3ccc(-n4ccnc4)nn3)c(C(=O)O)cc2F)c(F)cc1C(=O)O)c1ccc(-n2ccnc2)nn1] | +++ | +++ |
| 188 | 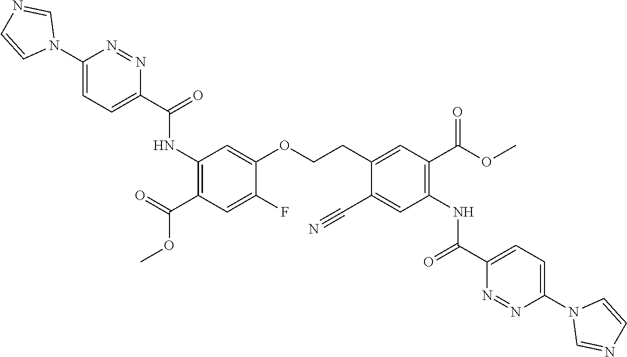 [SMILES: COC(=O)c1cc(F)c(OCCc2cc(C(=O)OC)c(NC(=O)c3ccc(-n4ccnc4)nn3)cc2C#N)cc1NC(=O)c1ccc(-n2ccnc2)nn1] | + | |
| 189 | 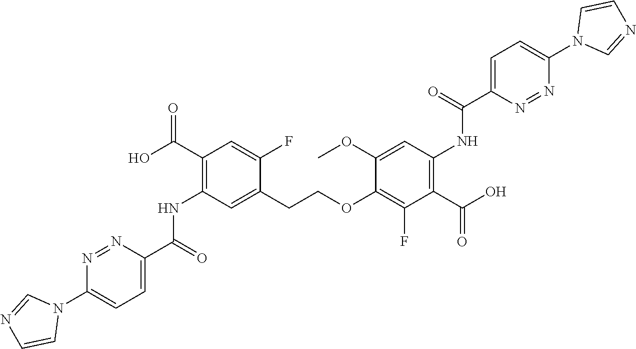 [SMILES: COc1cc(NC(=O)c2ccc(-n3ccnc3)nn2)c(C(=O)O)c(F)c1OCCc1cc(NC(=O)c2ccc(-n3ccnc3)nn2)c(C(=O)O)cc1F] | − | ++ |
| 190 | 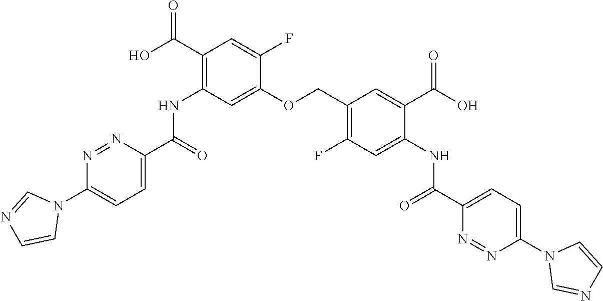 [SMILES: O=C(Nc1cc(OCc2cc(C(=O)O)c(NC(=O)c3ccc(-n4ccnc4)nn3)cc2F)c(F)cc1C(=O)O)c1ccc(-n2ccnc2)nn1] | + | + |
| 191 | 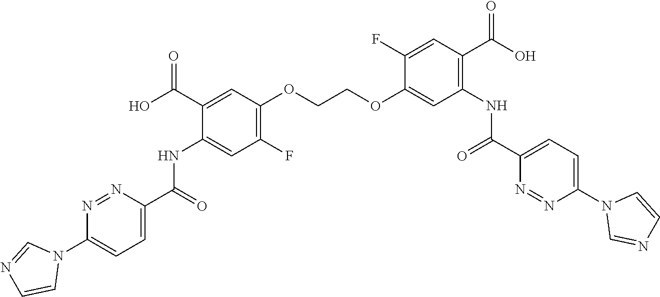 [SMILES: O=C(Nc1cc(OCCOc2cc(C(=O)O)c(NC(=O)c3ccc(-n4ccnc4)nn3)cc2F)c(F)cc1C(=O)O)c1ccc(-n2ccnc2)nn1] | +++ | ++++ |
| 192 | 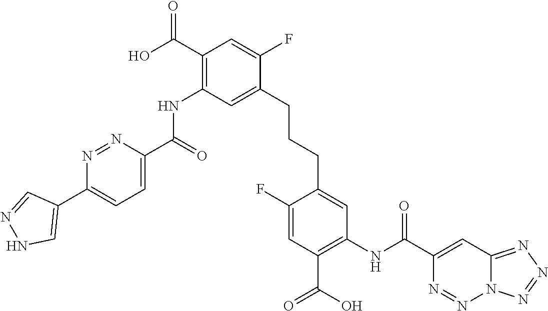 [SMILES: O=C(Nc1cc(CCCc2cc(NC(=O)c3cc4nnnn4nn3)c(C(=O)O)cc2F)c(F)cc1C(=O)O)c1ccc(-c2cn[nH]c2)nn1] | ++ | +++ |
| 193 | 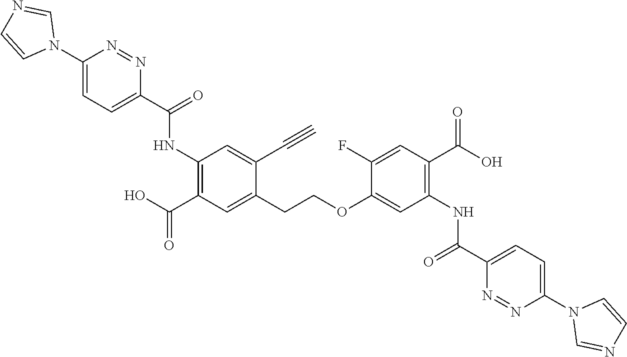 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1F] | | |
| 194 | 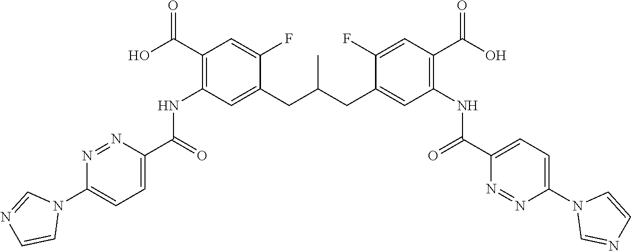 [SMILES: CC(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] | +++ | ++++ |
| 195 | 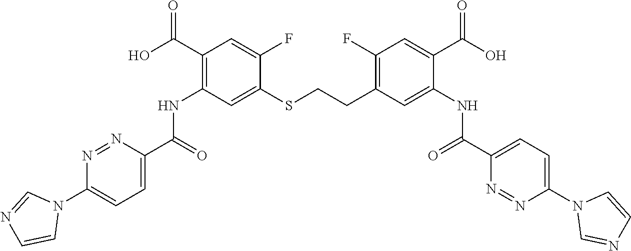 [SMILES: O=C(Nc1cc(CCSc2cc(NC(=O)c3ccc(-n4ccnc4)nn3)c(C(=O)O)cc2F)c(F)cc1C(=O)O)c1ccc(-n2ccnc2)nn1] | ++++ | ++++ |
| 196 | 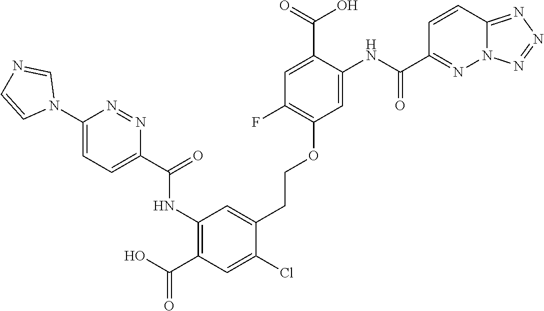 [SMILES: O=C(Nc1cc(CCOc2cc(NC(=O)c3ccc4nnnn4n3)c(C(=O)O)cc2F)c(Cl)cc1C(=O)O)c1ccc(-n2ccnc2)nn1] | +++ | +++ |
| 197 | 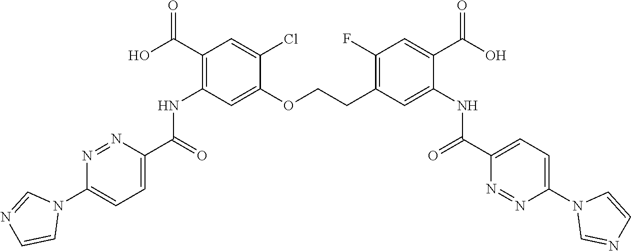 [SMILES: O=C(Nc1cc(CCOc2cc(NC(=O)c3ccc(-n4ccnc4)nn3)c(C(=O)O)cc2Cl)c(F)cc1C(=O)O)c1ccc(-n2ccnc2)nn1] | +++ | +++ |
| 198 | 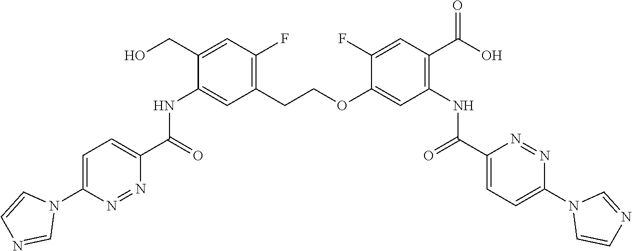 [SMILES: O=C(Nc1cc(CCOc2cc(NC(=O)c3ccc(-n4ccnc4)nn3)c(C(=O)O)cc2F)c(F)cc1CO)c1ccc(-n2ccnc2)nn1] | − | ++ |
| 199 | 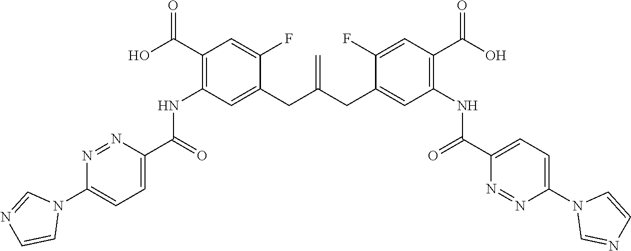 [SMILES: C=C(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] | +++ | ++++ |
| 200 | 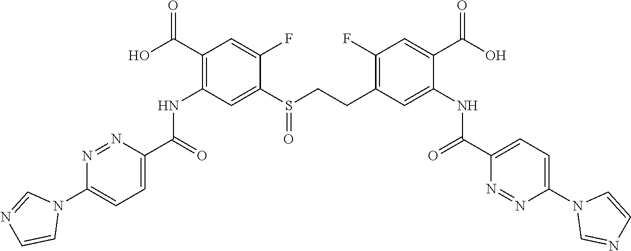 [SMILES: O=C(Nc1cc(CCS(=O)c2cc(NC(=O)c3ccc(-n4ccnc4)nn3)c(C(=O)O)cc2F)c(F)cc1C(=O)O)c1ccc(-n2ccnc2)nn1] | − | + |
| 201 | 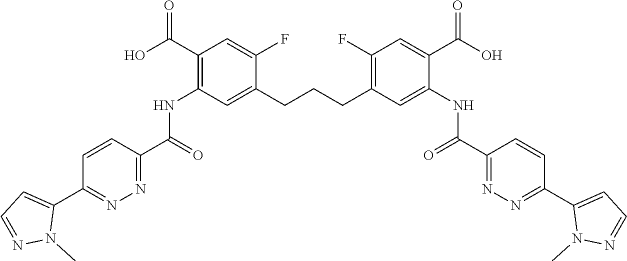 | − | ++ |
| 202 | 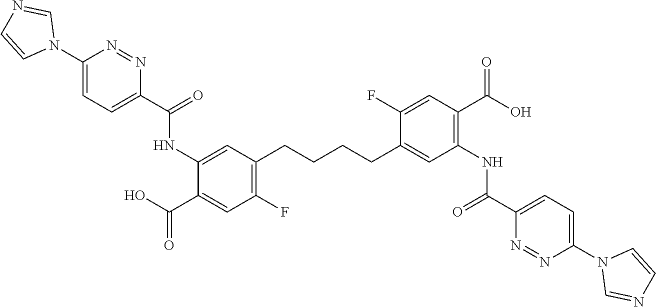 [SMILES: O=C(Nc1cc(CCCCc2cc(NC(=O)c3ccc(-n4ccnc4)nn3)c(C(=O)O)cc2F)c(F)cc1C(=O)O)c1ccc(-n2ccnc2)nn1] | +++ | +++ |
| 203 | 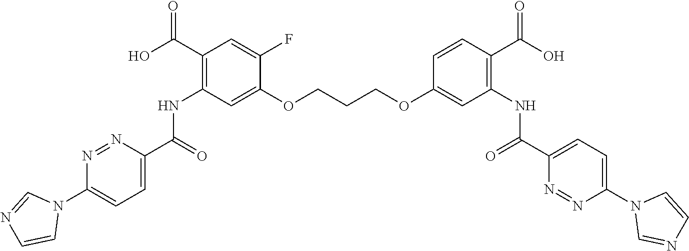 [SMILES: O=C(Nc1cc(OCCCOc2cc(NC(=O)c3ccc(-n4ccnc4)nn3)c(C(=O)O)cc2F)ccc1C(=O)O)c1ccc(-n2ccnc2)nn1] | ++ | +++ |
| 204 | 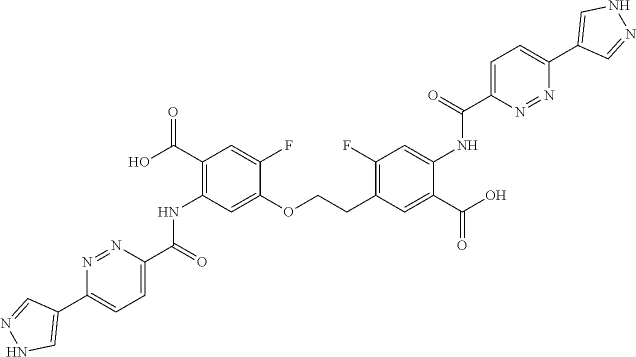 | +++ | ++++ |
| 205 | 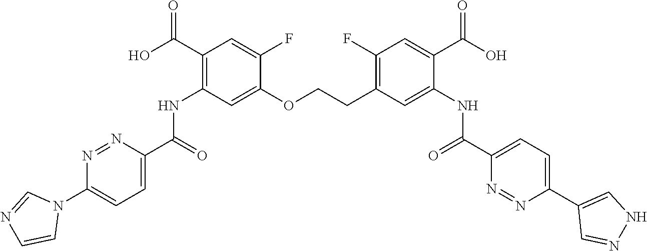 [SMILES: O=C(Nc1cc(CCOc2cc(NC(=O)c3ccc(-n4ccnc4)nn3)c(C(=O)O)cc2F)c(F)cc1C(=O)O)c1ccc(-c2cn[nH]c2)nn1] | ++++ | ++++ |
| 206 | 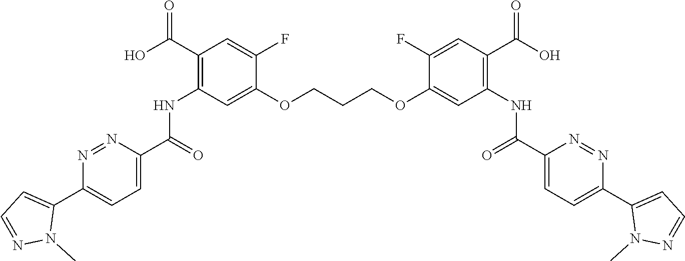 [SMILES: Cn1nccc1-c1ccc(C(=O)Nc2cc(OCCCOc3cc(NC(=O)c4ccc(-c5ccnn5C)nn4)c(C(=O)O)cc3F)c(F)cc2C(=O)O)nn1] | − | + |
| 207 | 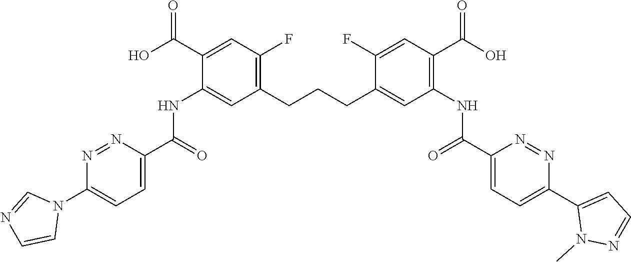 [SMILES: Cn1nccc1-c1ccc(C(=O)Nc2cc(CCCc3cc(NC(=O)c4ccc(-n5ccnc5)nn4)c(C(=O)O)cc3F)c(F)cc2C(=O)O)nn1] | − | + |
| 208 | 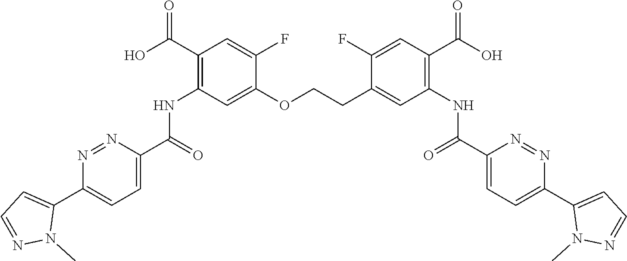 [SMILES: Cn1nccc1-c1ccc(C(=O)Nc2cc(CCOc3cc(NC(=O)c4ccc(-c5ccnn5C)nn4)c(C(=O)O)cc3F)c(F)cc2C(=O)O)nn1] | − | ++ |
| 209 | 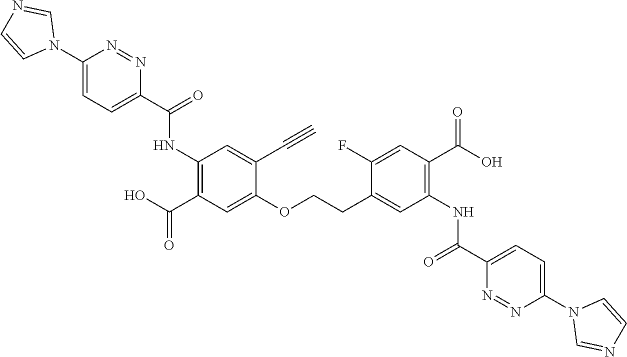 [SMILES: C#Cc1cc(NC(=O)c2ccc(-n3ccnc3)nn2)c(C(=O)O)cc1OCCc1cc(NC(=O)c2ccc(-n3ccnc3)nn2)c(C(=O)O)cc1F] | ++++ | − |
| 210 | 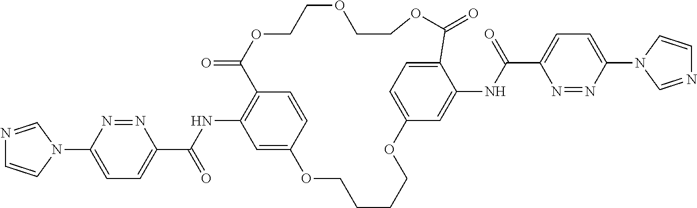 [SMILES: O=C(Nc1cc2ccc1C(=O)OCCOCCOC(=O)c1ccc(cc1NC(=O)c1ccc(-n3ccnc3)nn1)OCCCCO2)c1ccc(-n2ccnc2)nn1] | − | |
| 211 | 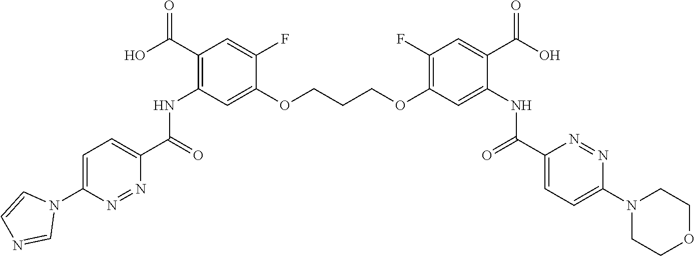 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCOCC2)nn1] | − | ++ |
| 212 | 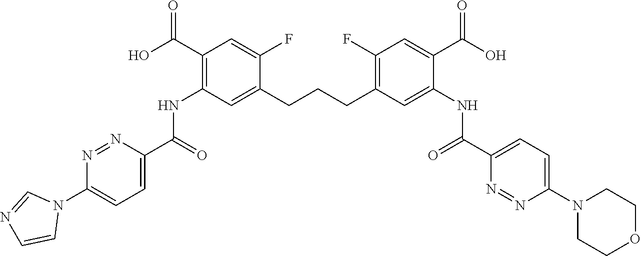 [SMILES: O=C(Nc1cc(CCCc2cc(NC(=O)c3ccc(-n4ccnc4)nn3)c(C(=O)O)cc2F)c(F)cc1C(=O)O)c1ccc(N2CCOCC2)nn1] | − | + |
| 213 | 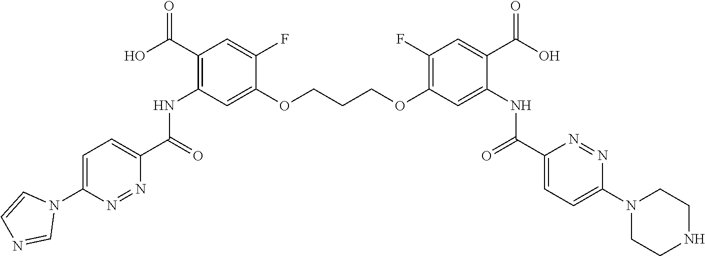 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCNCC2)nn1] | | |
| 214 | 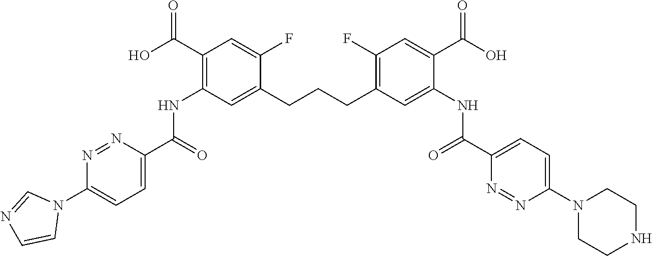 [SMILES: O=C(Nc1cc(CCCc2cc(NC(=O)c3ccc(-n4ccnc4)nn3)c(C(=O)O)cc2F)c(F)cc1C(=O)O)c1ccc(N2CCNCC2)nn1] | − | + |
| 215 | 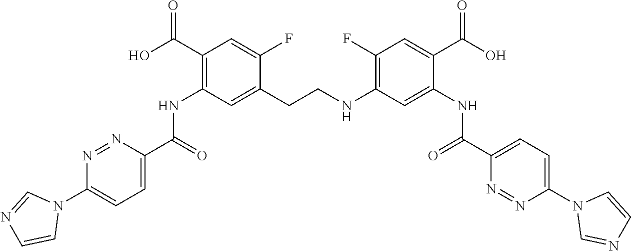 [SMILES: O=C(Nc1cc(CCNc2cc(NC(=O)c3ccc(-n4ccnc4)nn3)c(C(=O)O)cc2F)c(F)cc1C(=O)O)c1ccc(-n2ccnc2)nn1] | | |
| 216 | 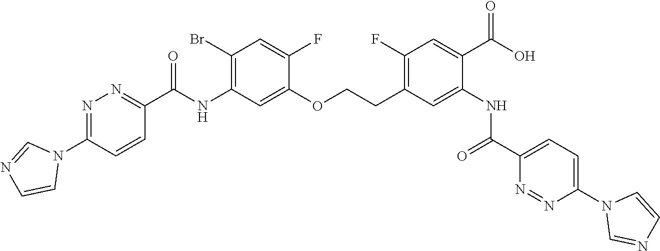 [SMILES: O=C(Nc1cc(OCCc2cc(NC(=O)c3ccc(-n4ccnc4)nn3)c(C(=O)O)cc2F)c(F)cc1Br)c1ccc(-n2ccnc2)nn1] | − | |
| 217 | 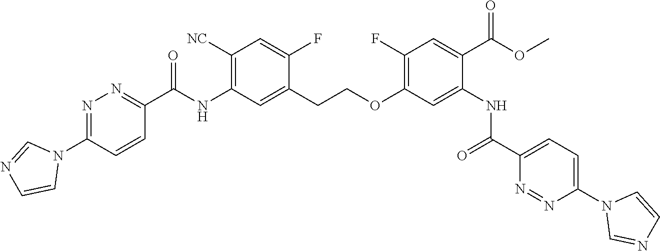 [SMILES: COC(=O)c1cc(F)c(OCCc2cc(NC(=O)c3ccc(-n4ccnc4)nn3)c(C#N)cc2F)cc1NC(=O)c1ccc(-n2ccnc2)nn1] | − | |
| 218 | 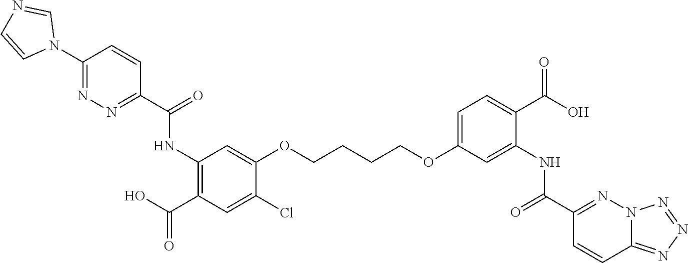 [SMILES: O=C(Nc1cc(OCCCCOc2ccc(C(=O)O)c(NC(=O)c3ccc4nnnn4n3)c2)c(Cl)cc1C(=O)O)c1ccc(-n2ccnc2)nn1] | | |
| 219 | 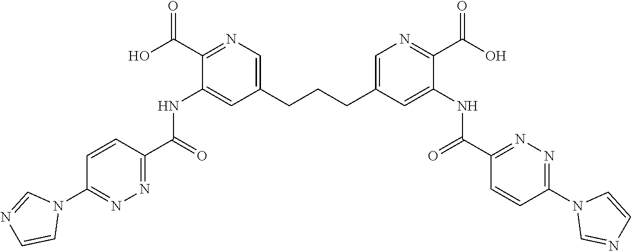 [SMILES: O=C(Nc1cc(CCCc2cnc(C(=O)O)c(NC(=O)c3ccc(-n4ccnc4)nn3)c2)cnc1C(=O)O)c1ccc(-n2ccnc2)nn1] | | |
| 220 | 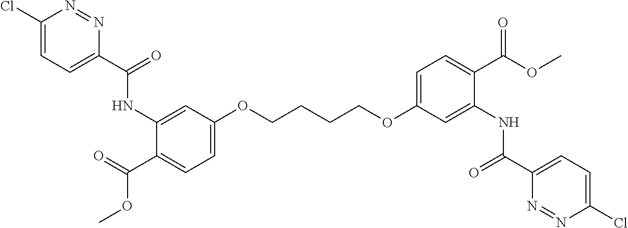 [SMILES: COC(=O)c1ccc(OCCCCOc2ccc(C(=O)OC)c(NC(=O)c3ccc(Cl)nn3)c2)cc1NC(=O)c1ccc(Cl)nn1] | − | |
| 221 | 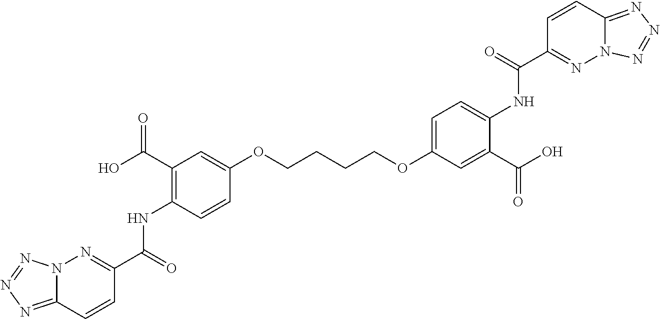 [SMILES: O=C(Nc1ccc(OCCCCOc2ccc(NC(=O)c3ccc4nnnn4n3)c(C(=O)O)c2)cc1C(=O)O)c1ccc2nnnn2n1] | − | − |
| 222 | 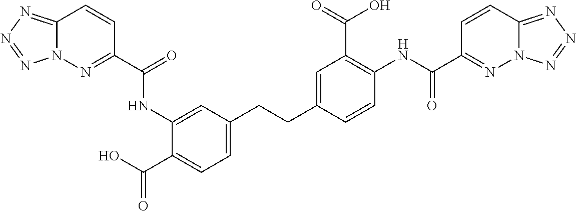 [SMILES: O=C(Nc1cc(CCc2ccc(NC(=O)c3ccc4nnnn4n3)c(C(=O)O)c2)ccc1C(=O)O)c1ccc2nnnn2n1] | − | − |
| 223 | 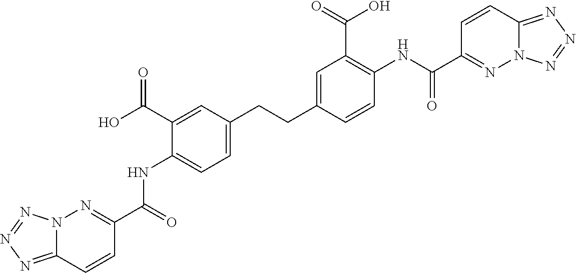 [SMILES: O=C(Nc1ccc(CCc2ccc(NC(=O)c3ccc4nnnn4n3)c(C(=O)O)c2)cc1C(=O)O)c1ccc2nnnn2n1] | − | − |
| 224 | 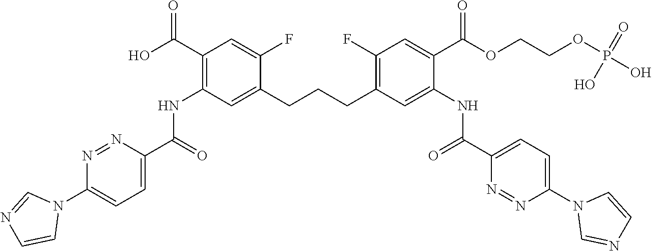 [SMILES: O=C(Nc1cc(CCCc2cc(NC(=O)c3ccc(-n4ccnc4)nn3)c(C(=O)OCCOP(=O)(O)O)cc2F)c(F)cc1C(=O)O)c1ccc(-n2ccnc2)nn1] | | |
| 225 | 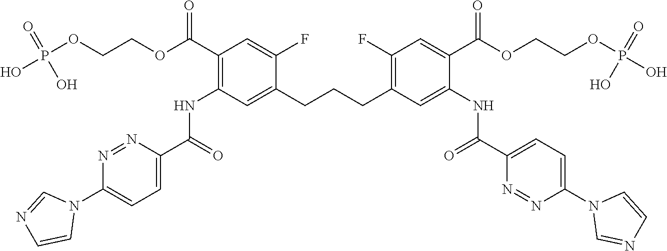 [SMILES: O=C(Nc1cc(CCCc2cc(NC(=O)c3ccc(-n4ccnc4)nn3)c(C(=O)OCCOP(=O)(O)O)cc2F)c(F)cc1C(=O)OCCOP(=O)(O)O)c1ccc(-n2ccnc2)nn1] | | |
| 226 | 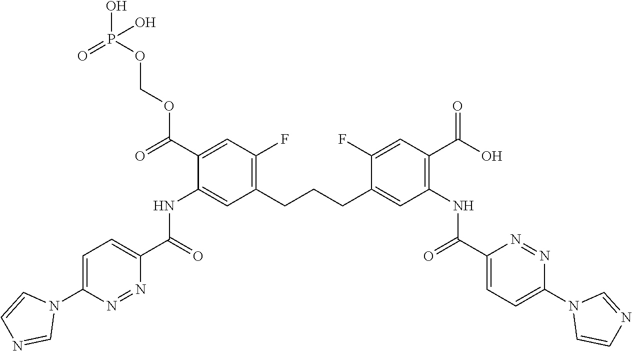 [SMILES: O=C(Nc1cc(CCCc2cc(NC(=O)c3ccc(-n4ccnc4)nn3)c(C(=O)OCOP(=O)(O)O)cc2F)c(F)cc1C(=O)O)c1ccc(-n2ccnc2)nn1] | | |
| 227 | 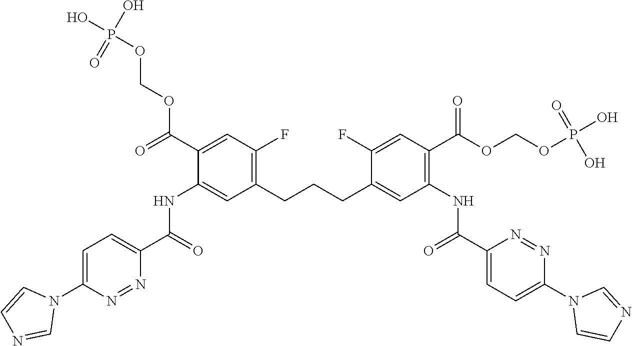 [SMILES: O=C(Nc1cc(CCCc2cc(NC(=O)c3ccc(-n4ccnc4)nn3)c(C(=O)OCOP(=O)(O)O)cc2F)c(F)cc1C(=O)OCOP(=O)(O)O)c1ccc(-n2ccnc2)nn1] | | |
| 228 | 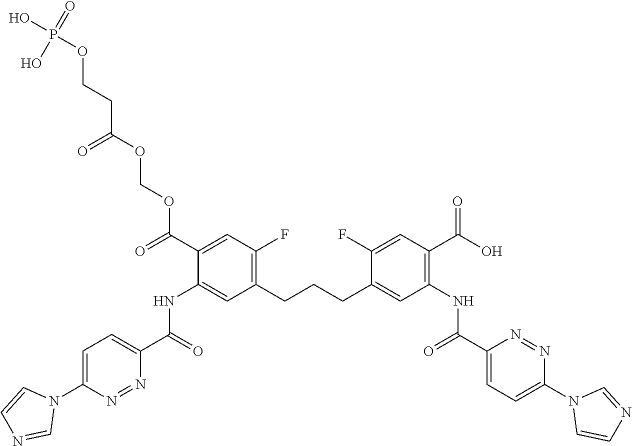 [SMILES: O=C(CCOP(=O)(O)O)OCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] | | |
| 229 | 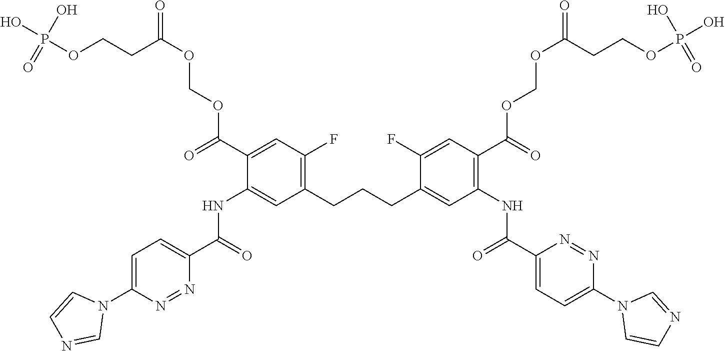 [SMILES: O=C(CCOP(=O)(O)O)OCOC(=O)c1cc(F)c(CCCc2cc(NC(=O)c3ccc(-n4ccnc4)nn3)c(C(=O)OCOC(=O)CCOP(=O)(O)O)cc2F)cc1NC(=O)c1ccc(-n2ccnc2)nn1] | | |
| 230 | 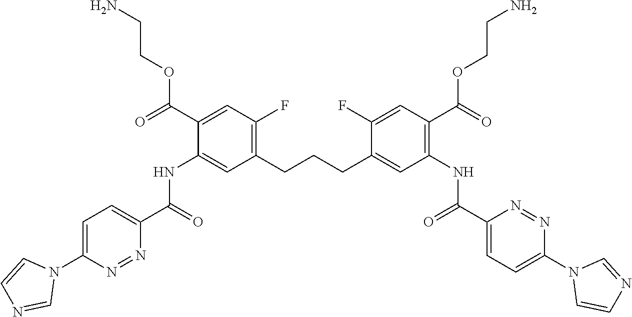 [SMILES: NCCOC(=O)c1cc(F)c(CCCc2cc(NC(=O)c3ccc(-n4ccnc4)nn3)c(C(=O)OCCN)cc2F)cc1NC(=O)c1ccc(-n2ccnc2)nn1] | | |
| 231 | 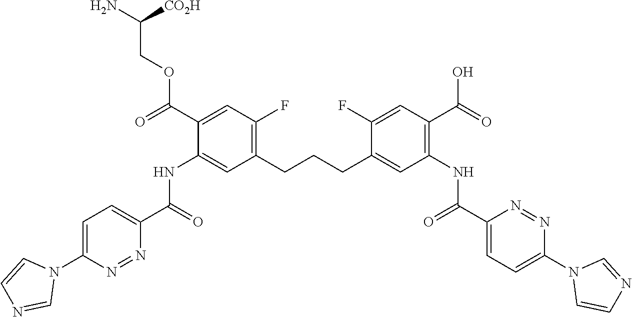 [SMILES: N[C@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] | | |
| 232 | 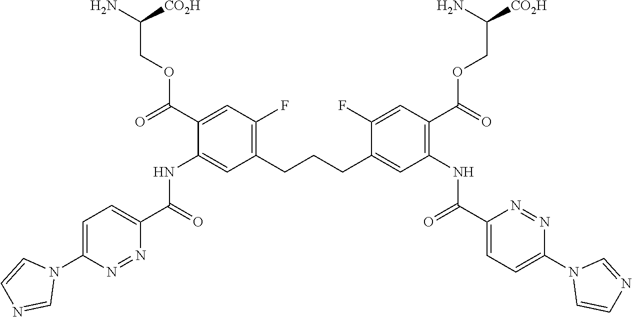 | | |
| 233 | 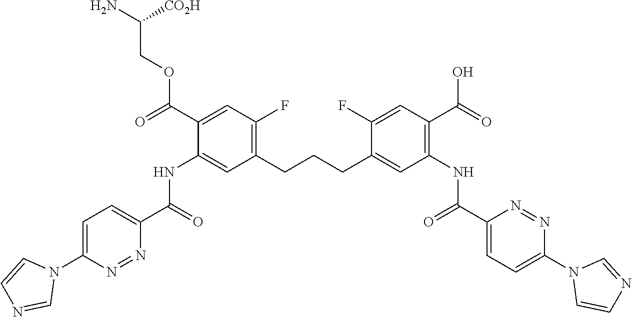 [SMILES: N[C@@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] | | |
| 234 | 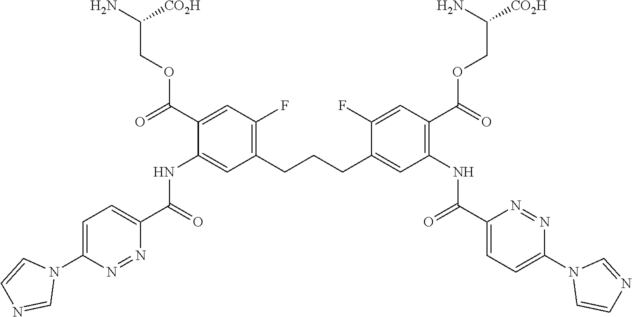 | | |
| 235 | 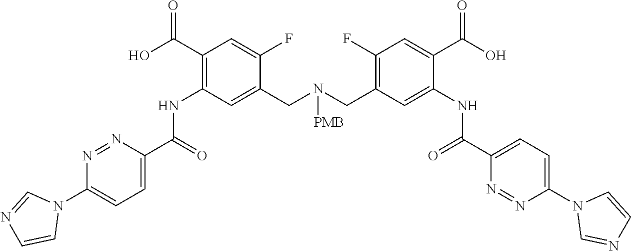 | | |
| 236 | 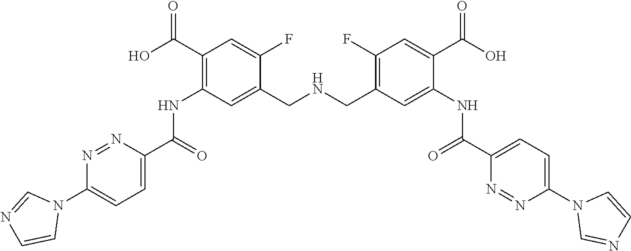 [SMILES: O=C(Nc1cc(CNCc2cc(NC(=O)c3ccc(-n4ccnc4)nn3)c(C(=O)O)cc2F)c(F)cc1C(=O)O)c1ccc(-n2ccnc2)nn1] | | |
| 237 | 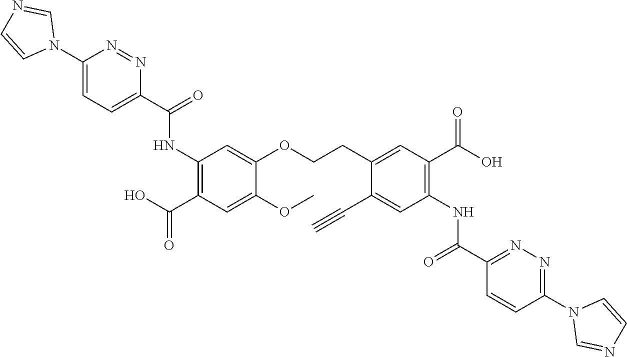 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1OC] | | |
| 238 | 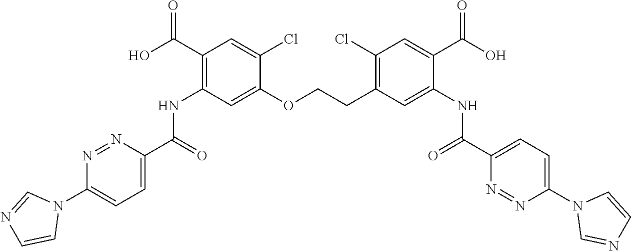 [SMILES: O=C(Nc1cc(CCOc2cc(NC(=O)c3ccc(-n4ccnc4)nn3)c(C(=O)O)cc2Cl)c(Cl)cc1C(=O)O)c1ccc(-n2ccnc2)nn1] | | |
| 239 | 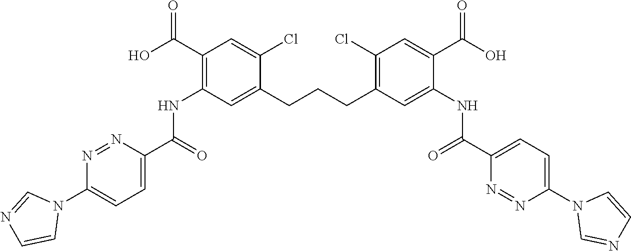 [SMILES: O=C(Nc1cc(CCCc2cc(NC(=O)c3ccc(-n4ccnc4)nn3)c(C(=O)O)cc2Cl)c(Cl)cc1C(=O)O)c1ccc(-n2ccnc2)nn1] | | |

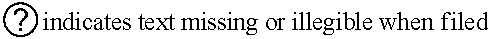

We claim:

1. A compound of Formula (I), or a pharmaceutically acceptable salt thereof: 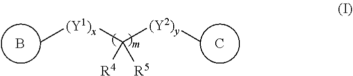
wherein
rings B and C are independently selected from Het, formula (a) and formula (b): 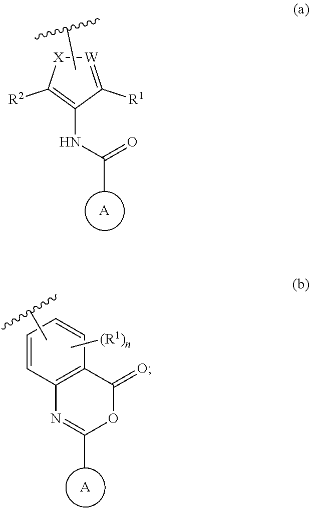
each ring A is optionally substituted by 1 to 4 R^{A} and is independently selected from:
a 5- or 6-membered monocyclic heteroaryl comprising 1 to 3 heteroatoms selected from O, S, and N, and
an 8- to 10-membered bicyclic heteroaryl comprising 1 to 6 heteroatoms selected from O, S, and N;
Het is an 8- to 10-membered bicyclic heteroaryl comprising 1 to 6 heteroatoms selected from O, S, and N and that is optionally substituted by 1 to 4 R^{A};
X is N, S, —N═C(R¹)—, or —C(R³)═C(R³)—;
W is —N═ or —C(R³)═,
Y¹ is selected from —O—, —CR₄R₅—, —(CH₂)_{L1}—O—, —(CH₂)_{L1}—S(O)₀₋₂— (wherein L1 is an integer selected from 1, 2, 3, 4, and 5); and —(CH₂)_{L1}—N(R^{L})— (wherein R^{L} is selected from H, C₁-C₆-alkyl, benzyl optionally substituted by 1 or 2 methoxy);
Y² is selected from —O—, —CR₄R₅—, —O—(CH₂)_{L1}—, —S(O)₀₋₂—(CH₂)_{L1}— (wherein L1 is an integer selected from 1, 2, 3, 4, and 5); and —N(R^{L})—(CH₂)_{L1}— (wherein R^{L} is H or C₁₂-C₆-alkyl);
m is an integer selected from 0, 1, 2, 3, 4, 5, and 6;
n is an integer selected from 0, 1, and 2;
x and y are integers independently selected from 0 and 1, wherein Y¹ and Y² are not simultaneously —O— when m is 0 and each of x and y is 1;
each R¹ and R³ is independently selected from the group consisting of H, halo, C₁-C₆-alkyl, C₂-C₆-alkenyl, C₂-C₆-alkynyl, C₁-C₆-alkoxyl, cyano, C₁-C₆-haloalkyl, and 3- to 10-membered heterocyclyl (wherein 1-4 heterocycloalkyl members are independently selected from N, O, and S), wherein any alkyl, alkenyl, alkynyl, alkoxyl, or heterocyclyl is optionally substituted by 1 to 4 R^{A};
R² is selected from the group consisting of —C(O)OR, —(C₁-C₆-alkyl)C(O)OR, C₁-C₆-haloalkyl, —P(O)(OR)₂, —C(O)NHR, halo, —CN, C₃-C₆-cycloalkenyl, 3- to 10-membered heterocyclyl (wherein 1-4 heterocycloalkyl members are independently selected from N, O, and S), and 5- to 10-membered heteroaryl (wherein 1-4 heteroaryl members are independently selected from N, O, and S), wherein any alkyl, cycloalkenyl, heterocyclyl, or heteroaryl is optionally substituted by 1 to 4 R^{A};
R is selected from the group consisting of H; C₁-C₆-alkyl optionally substituted with —((C₁-C₆-alkyl)OC(O)OC₁-C₆-alkyl), —OP(O)(OH)₂, —OC(O)(C₁-C₆-alkyl)-O—P(O)(OH)₂, —NH₂, —CH(NH₂)COOH, or 3- to 10-membered heterocyclyl (wherein 1-4 heterocycloalkyl members are independently selected from N, O, and S); and —(C₁-C₆-alkyl)(C₆-C₁₀-aryl);
each R⁴ and R⁵ is independently selected from the group consisting of H, halo, C₁-C₆-alkyl, and C₃-C₇-cycloalkyl, wherein
optionally any two R⁴ and R⁵ bound to the same carbon atom, together with the carbon atom to which they are bound, represent a C₃-C₅-cycloalkyl optionally substituted by 1 to 3 R^{A}, or they represent a C₂-C₆-alkenyl; and
optionally any two of R⁴ and R⁵ not bound to the same carbon atom, together with the respective carbon atoms to which they are bound, represent a C₃-C₇-cycloalkyl optionally substituted by 1 to 3 R^{A};
each R^{A} is independently selected from the group consisting of H, halo, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₁-C₆-alkyl)COOH, —C(O)(C₁-C₆-alkyl)C(O)(C₁-C₆-alkoxy), —C(O)N(H or C₁-C₆-alkyl)₂, —C(O)(C₃-C₁₄-cycloalkyl), —C₃-C₁₄-cycloalkyl, —(C₁-C₆-alkyl)(C₃-C₁₄-cycloalkyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S) that is optionally substituted with C₁-C₆-alkyl.

2. The compound or pharmaceutically acceptable salt thereof according to claim 1, wherein:
Y¹ and Y² are independently selected from —O— and —CR₄R₅—;
each R¹ and R³ is independently selected from the group consisting of H, halo, C₁-C₆-alkyl, C₂-C₆-alkenyl, C₂-C₆-alkynyl, C₁-C₆-alkoxyl, cyano, and C₁-C₆-haloalkyl, wherein any alkyl, alkenyl, alkynyl or alkoxyl is optionally substituted by 1 to 4 R^{A},
R² is selected from the group consisting of —C(O)OR, —C(O)NHR, C₃-C₆-cycloalkenyl, and 3- to 10-membered heterocyclyl, wherein any alkyl, cycloalkenyl, or heterocyclyl is optionally substituted by 1 to 4 R^{A};
R is selected from the group consisting of H, C₁-C₆-alkyl optionally substituted with —((C₁-C₆-alkyl)OC(O)OC₁-C₆-alkyl) or 3- to 10-membered heterocyclyl, and —(C₁-C₆-alkyl)(C₆-C₁₀-aryl);
each R⁴ and R⁵ is independently selected from the group consisting of H, halo, C₁-C₆-alkyl, and C₃-C₇-cycloalkyl, wherein
optionally any two R⁴ and R⁵ bound to the same carbon atom, together with the carbon atom to which they are bound, represent a C₃-C₅-cycloalkyl optionally substituted by 1 to 3 R^{A}; and
optionally any two of R⁴ and R⁵ not bound to the same carbon atom, together with the respective carbon atoms to which they are bound, represent a C₃-C₇-cycloalkyl optionally substituted by 1 to 3 R^{A}; and
each R^{A} is independently selected from the group consisting of H, halo, —CN, -hydroxy, oxo, C₁-C₆-alkyl, C₁-C₆-alkoxy, C₂-C₆-alkenyl, C₂-C₆-alkynyl, NH₂, —S(O)₀₋₂—(C₁-C₆-alkyl), —S(O)₀₋₂—(C₆-C₁₀-aryl), —C(O)(C₁-C₆-alkyl), —C(O)(C₁-C₆-alkyl)COOH, —C(O)(C₃-C₁₄-cycloalkyl), —C₃-C₁₄-cycloalkyl, —(C₁-C₆-alkyl)(C₃-C₄-cycloalkyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

3. The compound or pharmaceutically acceptable salt thereof according to claim 1, wherein ring B is the same as ring C.

4. The compound or pharmaceutically acceptable salt thereof according to claim 1, wherein ring B is different from ring C.

5. The compound or pharmaceutically acceptable salt thereof according to any one of claims 1 to 4, wherein each of rings B and C is of formula (a).

6. The compound or pharmaceutically acceptable salt thereof according to claim 5, wherein:
ring B is of formula (a), wherein ring A is a 5- or 6-membered monocyclic heteroaryl comprising 1 to 3 heteroatoms selected from O, S, and N, optionally substituted by 1 to 4 R^{A}; and
ring C is of formula (a), wherein ring A is an 8- to 10-membered bicyclic heteroaryl comprising 1 to 6 heteroatoms selected from O, S, and N, optionally substituted by 1 to 4 R^{A}.

7. The compound or pharmaceutically acceptable salt thereof according to claim 6, wherein the monocyclic heteroaryl is one selected from the group consisting of pyridinyl, pyridazinyl, pyrazinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl.

8. The compound or pharmaceutically acceptable salt thereof according to claim 6 or 7, wherein monocyclic heteroaryl is one selected from the group consisting of pyridinyl, pyridazinyl, pyrazinyl, and pyrimidinyl.

9. The compound or pharmaceutically acceptable salt thereof according to any one of claims 6 to 8, wherein the monocyclic heteroaryl is substituted by R^{A} that is a 5- to 10-membered heteroaryl (wherein 1-4 heteroaryl members are independently selected from N, O, and S).

10. The compound or pharmaceutically acceptable salt thereof according to claim 9, wherein the 5- to 10-membered heteroaryl is selected from tetrazolyl, imidazolyl, and triazolyl.

11. The compound or pharmaceutically acceptable salt thereof according to claim 6, wherein the 8- to 10-membered bicyclic heteroaryl is one selected from the group consisting of indolizinyl, benzothienyl, quinazolinyl, purinyl, indolyl, quinolinyl, tetrazolo[1,5-b]pyridazinyl, [1,2,3]triazolo[1,5-b]pyridazinyl, bicyclic[1,2,4]triazolo[1,5-a]pyrimidinyl, [1,2,4]triazolo[4,3-a]pyrimidinyl, and imidazo[1,2-a]pyrimidinyl.

12. The compound or pharmaceutically acceptable salt thereof according to claim 5, wherein ring B and ring C are the same and are of formula (a), wherein ring A is a 5- or 6-membered monocyclic heteroaryl comprising 1 to 3 heteroatoms selected from O, S, and N, optionally substituted by 1 to 4 R^{A}.

13. The compound or pharmaceutically acceptable salt thereof according to claim 12, wherein the monocyclic heteroaryl is one selected from the group consisting of pyridinyl, pyridazinyl, pyrazinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, and furanyl.

14. The compound or pharmaceutically acceptable salt thereof according to claim 5, wherein ring B and ring C are the same and are of formula (a), wherein ring A is an 8- to 10-membered bicyclic heteroaryl.

15. The compound or pharmaceutically acceptable salt thereof according to claim 1, wherein ring B is Het optionally substituted by 1 to 4 R^{A} and ring C is of formula (a).

16. The compound or pharmaceutically acceptable salt thereof according to claim 15, wherein Het is selected from the group consisting of indolizinyl, benzothienyl, quinazolinyl, purinyl, indolyl, quinolinyl, tetrazolo[1,5-b]pyridazinyl, [1,2,3]triazolo[1,5-b]pyridazinyl, [1,2,4]triazolo[1,5-a]pyrimidinyl, [1,2,4]triazolo[4,3-a]pyrimidinyl, and imidazo[1,2-a]pyrimidinyl.

17. The compound or pharmaceutically acceptable salt thereof according to claim 15 or 16, wherein Het is benzothienyl optionally substituted by 1 to 4 R^{A} selected from the group consisting of halo, C₁-C₆-alkoxy, —C(O)(C₁-C₆-alkyl)COOH.

18. The compound or pharmaceutically acceptable salt according to any one of claims 1 to 17, wherein X is —C(R³)═C(R³)— and W is —C(R³)═.

19. The compound or pharmaceutically acceptable salt thereof according to any one of claims 1 to 18, wherein each R³ is independently selected from the group consisting of H, halo, and C₁-C₆-alkoxyl.

20. The compound or pharmaceutically acceptable salt thereof according to any one of claims 1 to 19, wherein R² is —C(O)OR and R is H or C₁-C₆-alkyl.

21. The compound or pharmaceutically acceptable salt according to any one of claims 1 to 20, wherein each of Y¹ and Y² is —O—, and each of x and y is 1.

22. The compound or pharmaceutically acceptable salt thereof according to claim 21, wherein m is 4.

23. The compound or pharmaceutically acceptable salt thereof according to any one of claims 1 to 20, wherein each of Y¹ and Y² is —CR₄R₅—, and each of x and y is 1.

24. The compound or pharmaceutically acceptable salt thereof according to claim 23, wherein m is 1.

25. The compound or pharmaceutically acceptable salt thereof according to any one of claims 1 to 24, wherein each R¹ is independently selected from H and halo.

26. The compound or pharmaceutically acceptable salt thereof according to claim 1, wherein:
ring B is of formula (a), wherein ring A is a 6-membered monocyclic heteroaryl comprising 1 to 3 heteroatoms selected from O, S, and N, and that is substituted by a 5- to 10-membered heteroaryl (wherein 1-4 heteroaryl members are independently selected from N, O, and S);
ring C is of formula (a), wherein ring A is an 8- to 10-membered bicyclic heteroaryl;
X is —C(R³)═C(R³)— and W is —C(R³)═, wherein each R³ is independently selected from H, halo, and C₁-C₆-alkoxyl;
R¹ is H;
R² is —C(O)OR and R is H or C₁-C₆-alkyl;
each R⁴ and R⁵ is H;
each of x and y is 1; and
each of Y¹ and Y² is —O— and m is 4, or each of Y¹ and Y² is —CH₂— and m is 1.

27. The compound or pharmaceutically acceptable salt thereof according to claim 1, wherein:
each of rings B and C is of formula (a), wherein each ring A is a 6-membered monocyclic heteroaryl comprising 1 to 3 heteroatoms selected from O, S, and N, and that is substituted by one R^{A} that is a 5- to 10-membered heteroaryl (wherein 1-4 heteroaryl members are independently selected from N, O, and S);
X is —C(R³)═C(R³)— and W is —C(R³)═, wherein each R³ is independently selected from H and halo;
R¹ is H;
R¹ is —C(O)OR and R is H;
each of x and y is 1;
m is 0 or 1;
Y¹ is —CR₄R₅— or —(CH₂)_{L1}—N(R^{L})—; and
Y² is —O— or —CR₄R₅—.

28. The compound or pharmaceutically acceptable salt thereof according to claim 27, wherein each ring A is pyridazinyl, and each R^{A} is imidazolyl.

29. The compound or pharmaceutically acceptable salt thereof according to claim 1, wherein the compound is one selected from the following table:

| | |
|---|---|
| 1 | 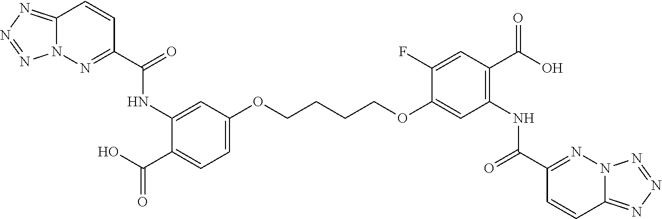 [SMILES: O=C(Nc1cc(OCCCCOc2cc(NC(=O)c3ccc4nnnn4n3)c(C(=O)O)cc2F)ccc1C(=O)O)c1ccc2nnnn2n1] |
| 2 | 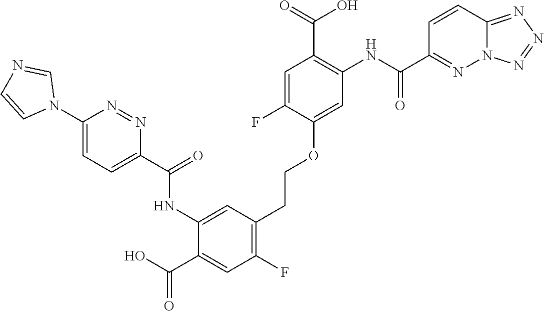 [SMILES: O=C(Nc1cc(CCOc2cc(NC(=O)c3ccc4nnnn4n3)c(C(=O)O)cc2F)c(F)cc1C(=O)O)c1ccc(-n2ccnc2)nn1] |
| 3 | 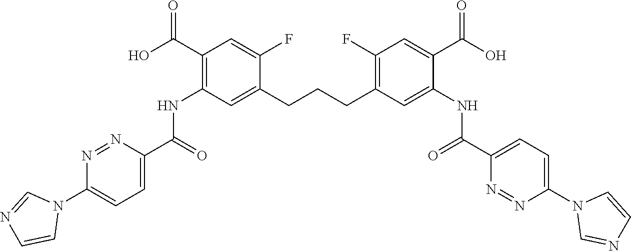 [SMILES: O=C(Nc1cc(CCCc2cc(NC(=O)c3ccc(-n4ccnc4)nn3)c(C(=O)O)cc2F)c(F)cc1C(=O)O)c1ccc(-n2ccnc2)nn1] |
| 4 | 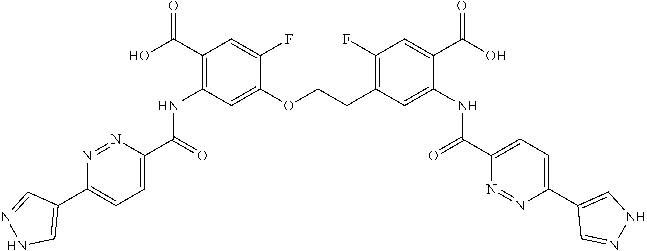 [SMILES: O=C(Nc1cc(CCOc2cc(NC(=O)c3ccc(-c4cn[nH]c4)nn3)c(C(=O)O)cc2F)c(F)cc1C(=O)O)c1ccc(-c2cn[nH]c2)nn1] |
| 5 | 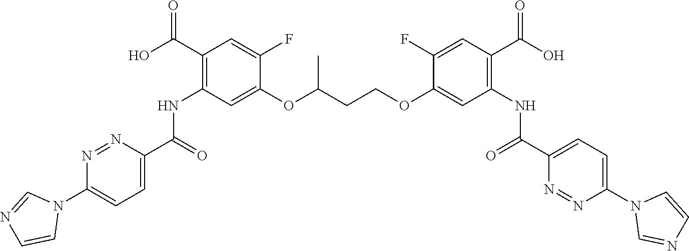 [SMILES: CC(CCOc1cc(NC(=O)c2ccc(-n3ccnc3)nn2)c(C(=O)O)cc1F)Oc1cc(NC(=O)c2ccc(-n3ccnc3)nn2)c(C(=O)O)cc1F] |
| 6 | 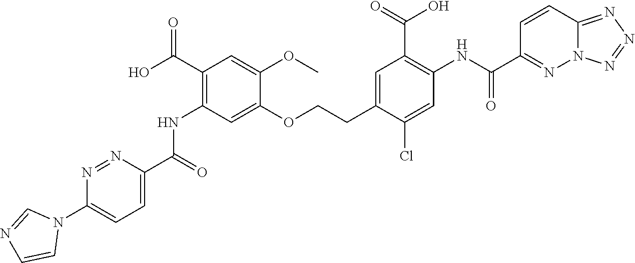 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1Cl] |
| 7 | 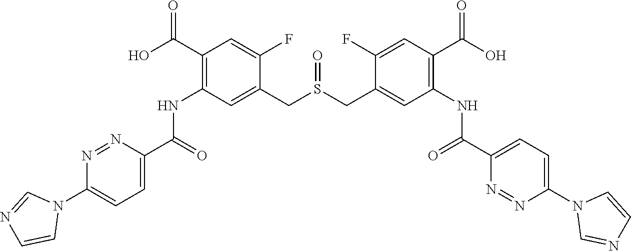 [SMILES: O=C(Nc1cc(CS(=O)Cc2cc(NC(=O)c3ccc(-n4ccnc4)nn3)c(C(=O)O)cc2F)c(F)cc1C(=O)O)c1ccc(-n2ccnc2)nn1] |
| 8 | 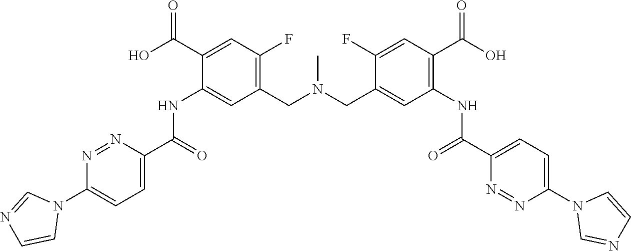 [SMILES: CN(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] |
| 9 | 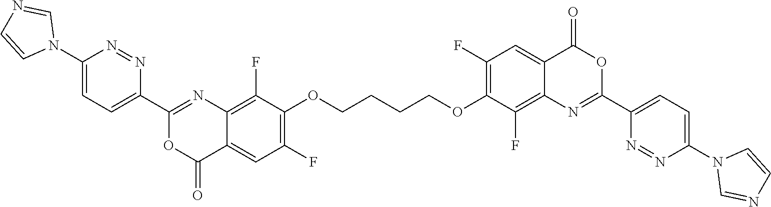 [SMILES: O=c1oc(-c2ccc(-n3ccnc3)nn2)nc2c(F)c(OCCCCOc3c(F)cc4c(=O)oc(-c5ccc(-n6ccnc6)nn5)nc4c3F)c(F)cc12] |
| 10 | 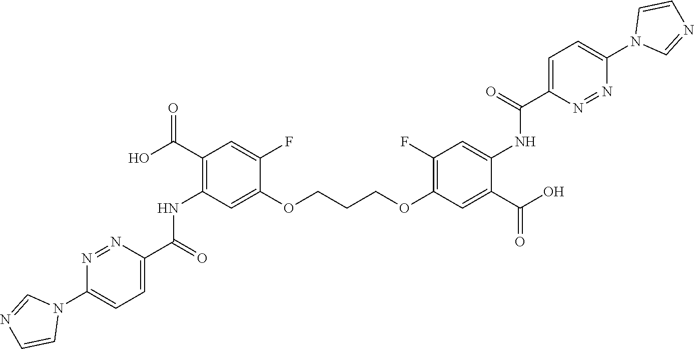 [SMILES: O=C(Nc1cc(OCCCOc2cc(C(=O)O)c(NC(=O)c3ccc(-n4ccnc4)nn3)cc2F)c(F)cc1C(=O)O)c1ccc(-n2ccnc2)nn1] |
| 11 | 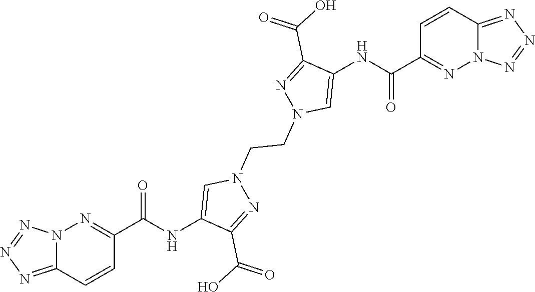 [SMILES: O=C(Nc1cn(CCn2cc(NC(=O)c3ccc4nnnn4n3)c(C(=O)O)n2)nc1C(=O)O)c1ccc2nnnn2n1] |
| 12 | 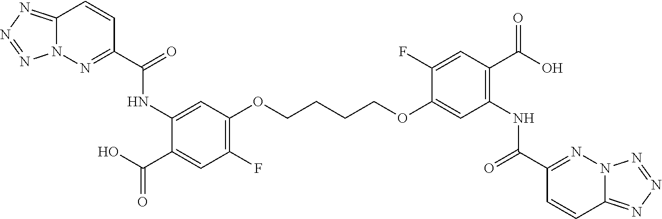 [SMILES: O=C(Nc1cc(OCCCCOc2cc(NC(=O)c3ccc4nnnn4n3)c(C(=O)O)cc2F)c(F)cc1C(=O)O)c1ccc2nnnn2n1] |
| 13 | 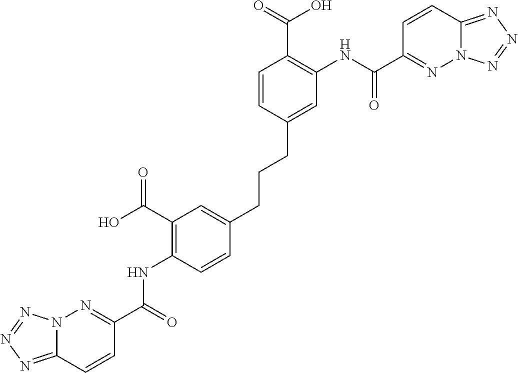 [SMILES: O=C(Nc1cc(CCCc2ccc(NC(=O)c3ccc4nnnn4n3)c(C(=O)O)c2)ccc1C(=O)O)c1ccc2nnnn2n1] |
| 14 | 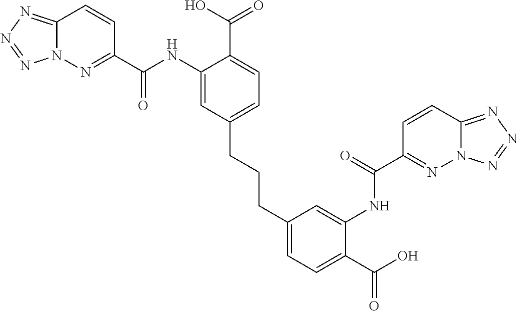 [SMILES: O=C(Nc1cc(CCCc2ccc(C(=O)O)c(NC(=O)c3ccc4nnnn4n3)c2)ccc1C(=O)O)c1ccc2nnnn2n1] |
| 15 | 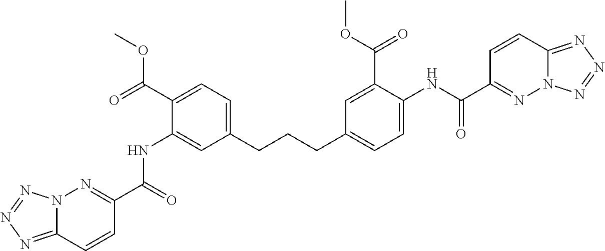 [SMILES: COC(=O)c1ccc(CCCc2ccc(NC(=O)c3ccc4nnnn4n3)c(C(=O)OC)c2)cc1NC(=O)c1ccc2nnnn2n1] |
| 16 | 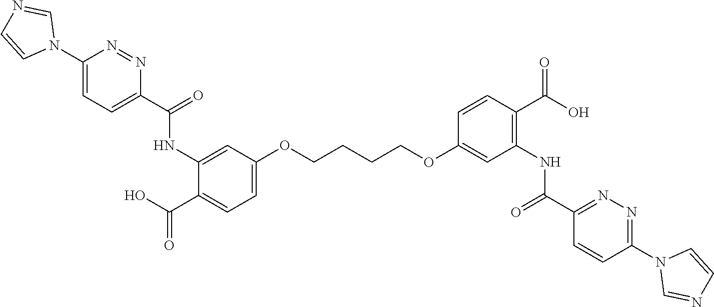 [SMILES: O=C(Nc1cc(OCCCCOc2ccc(C(=O)O)c(NC(=O)c3ccc(-n4ccnc4)nn3)c2)ccc1C(=O)O)c1ccc(-n2ccnc2)nn1] |
| 17 | 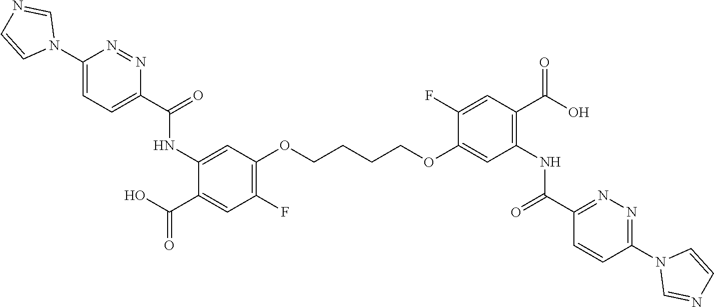 [SMILES: O=C(Nc1cc(OCCCCOc2cc(NC(=O)c3ccc(-n4ccnc4)nn3)c(C(=O)O)cc2F)c(F)cc1C(=O)O)c1ccc(-n2ccnc2)nn1] |
| 18 | 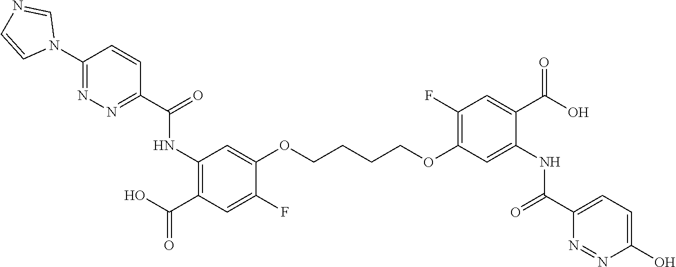 [SMILES: O=C(Nc1cc(OCCCCOc2cc(NC(=O)c3ccc(-n4ccnc4)nn3)c(C(=O)O)cc2F)c(F)cc1C(=O)O)c1ccc(O)nn1] |
| 19 | 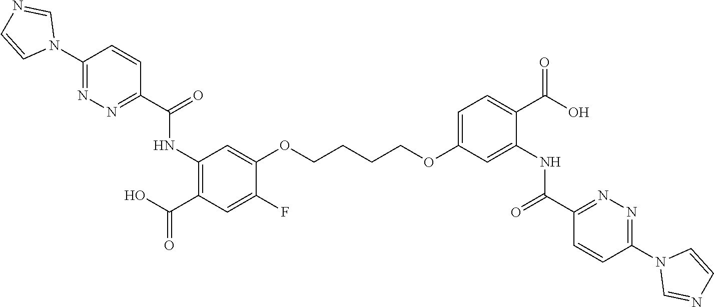 [SMILES: O=C(Nc1cc(OCCCCOc2cc(NC(=O)c3ccc(-n4ccnc4)nn3)c(C(=O)O)cc2F)ccc1C(=O)O)c1ccc(-n2ccnc2)nn1] |
| 20 | 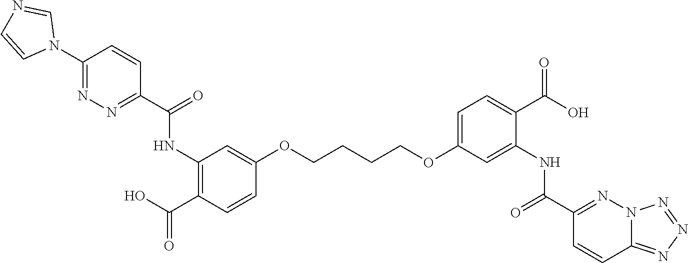 [SMILES: O=C(Nc1cc(OCCCCOc2ccc(C(=O)O)c(NC(=O)c3ccc4nnnn4n3)c2)ccc1C(=O)O)c1ccc(-n2ccnc2)nn1] |
| 21 | 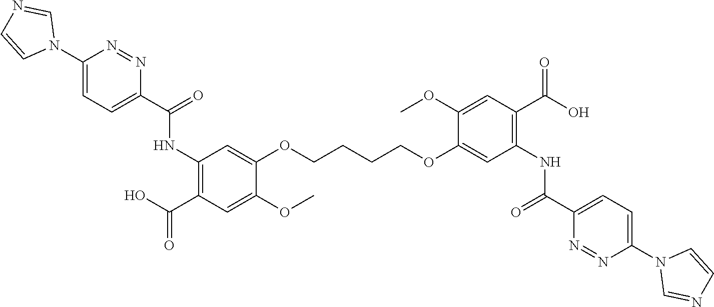 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1OC] |
| 22 | 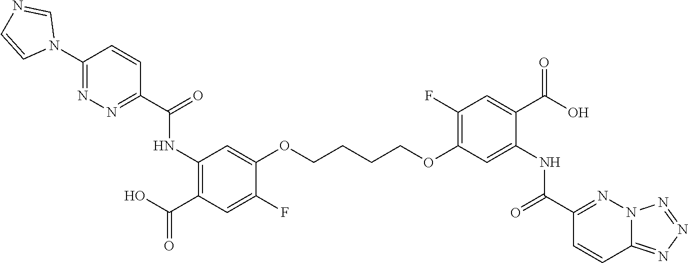 [SMILES: O=C(Nc1cc(OCCCCOc2cc(NC(=O)c3ccc4nnnn4n3)c(C(=O)O)cc2F)c(F)cc1C(=O)O)c1ccc(-n2ccnc2)nn1] |
| 23 | 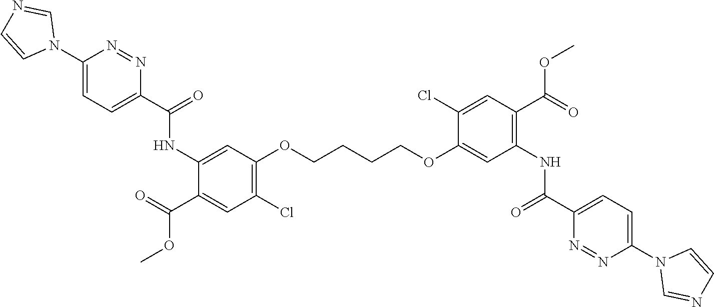 [SMILES: COC(=O)c1cc(Cl)c(OCCCCOc2cc(NC(=O)c3ccc(-n4ccnc4)nn3)c(C(=O)OC)cc2Cl)cc1NC(=O)c1ccc(-n2ccnc2)nn1] |
| 24 | 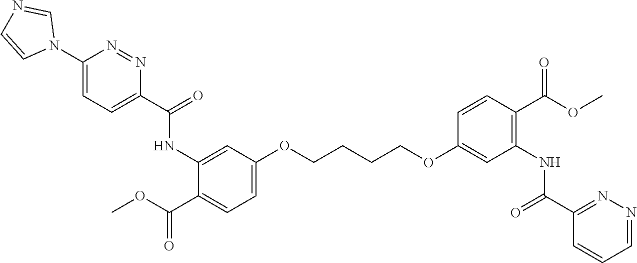 [SMILES: COC(=O)c1ccc(OCCCCOc2ccc(C(=O)OC)c(NC(=O)c3ccc(-n4ccnc4)nn3)c2)cc1NC(=O)c1cccnn1] |
| 25 | 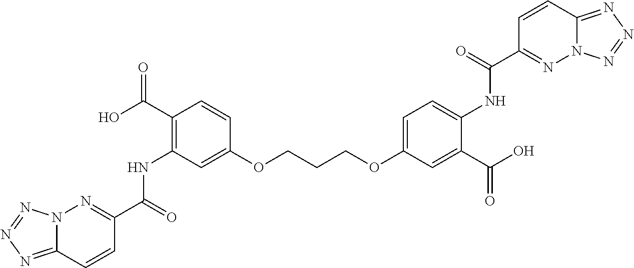 [SMILES: O=C(Nc1cc(OCCCOc2ccc(NC(=O)c3ccc4nnnn4n3)c(C(=O)O)c2)ccc1C(=O)O)c1ccc2nnnn2n1] |
| 26 | 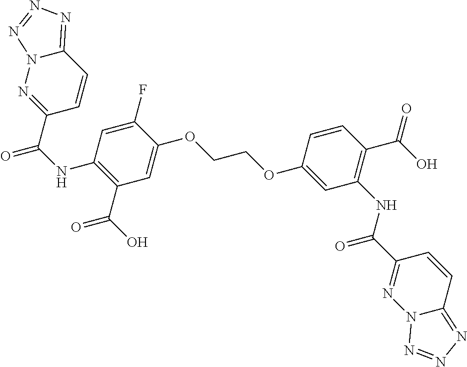 [SMILES: O=C(Nc1cc(OCCOc2cc(C(=O)O)c(NC(=O)c3ccc4nnnn4n3)cc2F)ccc1C(=O)O)c1ccc2nnnn2n1] |
| 27 | 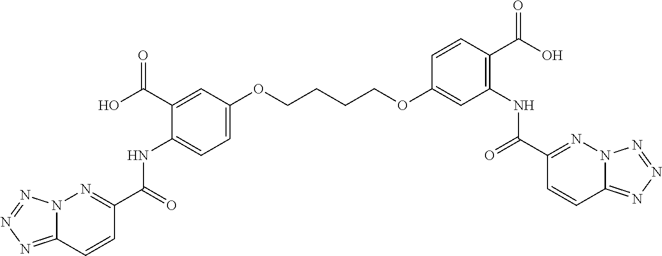 [SMILES: O=C(Nc1cc(OCCCCOc2ccc(NC(=O)c3ccc4nnnn4n3)c(C(=O)O)c2)ccc1C(=O)O)c1ccc2nnnn2n1] |
| 28 | 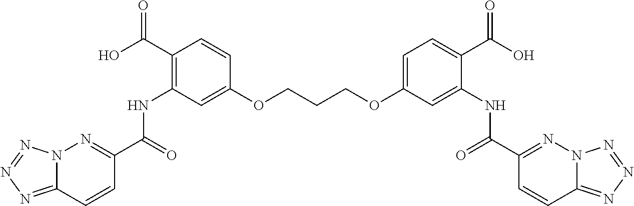 [SMILES: O=C(Nc1cc(OCCCOc2ccc(C(=O)O)c(NC(=O)c3ccc4nnnn4n3)c2)ccc1C(=O)O)c1ccc2nnnn2n1] |
| 29 | 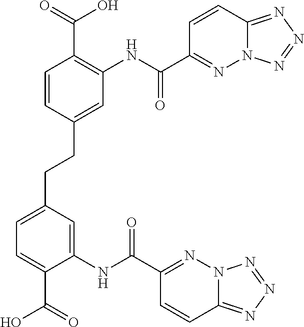 [SMILES: O=C(Nc1cc(CCc2ccc(C(=O)O)c(NC(=O)c3ccc4nnnn4n3)c2)ccc1C(=O)O)c1ccc2nnnn2n1] |
| 30 | 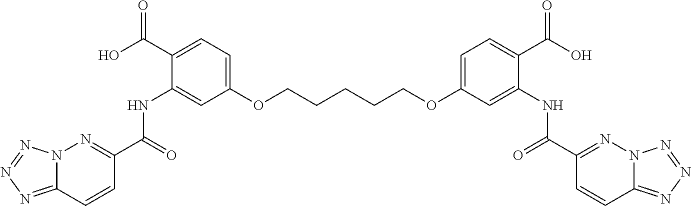 [SMILES: O=C(Nc1cc(OCCCCCOc2ccc(C(=O)O)c(NC(=O)c3ccc4nnnn4n3)c2)ccc1C(=O)O)c1ccc2nnnn2n1] |
| 31 | 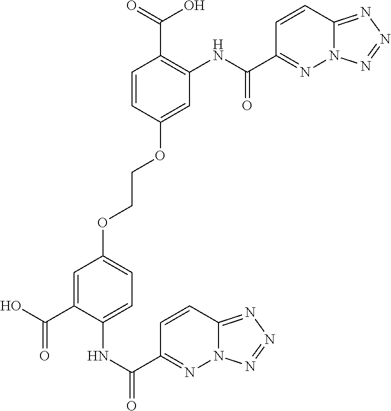 [SMILES: O=C(Nc1cc(OCCOc2ccc(NC(=O)c3ccc4nnnn4n3)c(C(=O)O)c2)ccc1C(=O)O)c1ccc2nnnn2n1] |
| 32 | 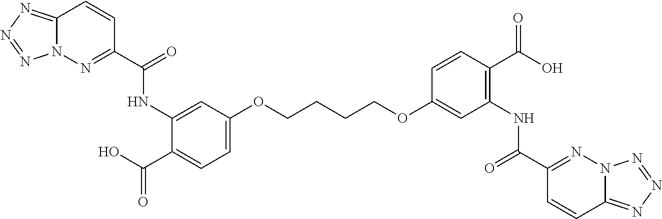 [SMILES: O=C(Nc1cc(OCCCCOc2ccc(C(=O)O)c(NC(=O)c3ccc4nnnn4n3)c2)ccc1C(=O)O)c1ccc2nnnn2n1] |
| 33 | 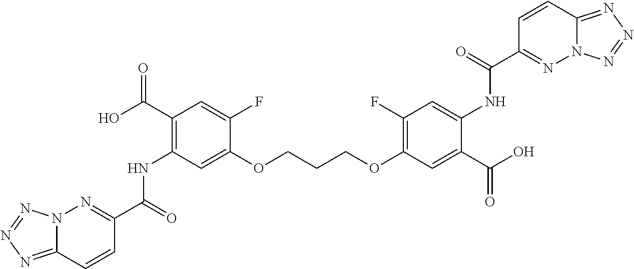 [SMILES: O=C(Nc1cc(OCCCOc2cc(C(=O)O)c(NC(=O)c3ccc4nnnn4n3)cc2F)c(F)cc1C(=O)O)c1ccc2nnnn2n1] |
| 34 | 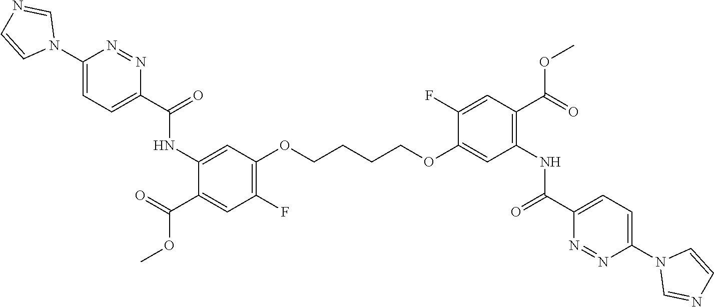 [SMILES: COC(=O)c1cc(F)c(OCCCCOc2cc(NC(=O)c3ccc(-n4ccnc4)nn3)c(C(=O)OC)cc2F)cc1NC(=O)c1ccc(-n2ccnc2)nn1] |
| 35 | 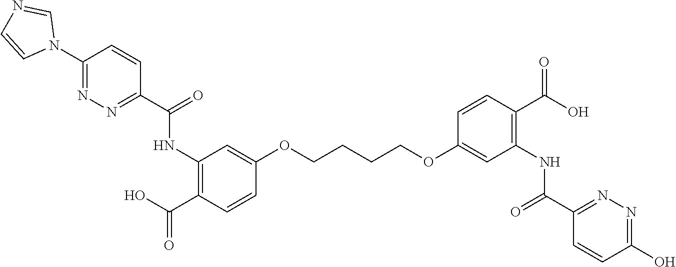 [SMILES: O=C(Nc1cc(OCCCCOc2ccc(C(=O)O)c(NC(=O)c3ccc(-n4ccnc4)nn3)c2)ccc1C(=O)O)c1ccc(O)nn1] |
| 36 | 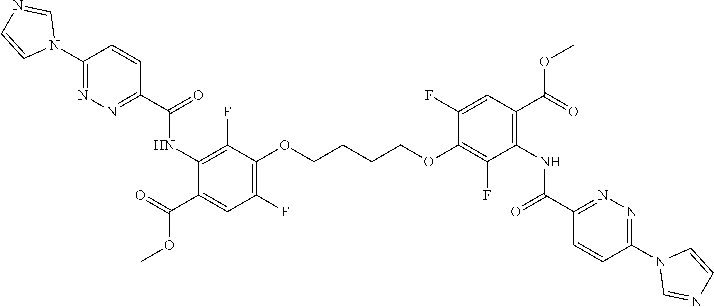 [SMILES: COC(=O)c1cc(F)c(OCCCCOc2c(F)cc(C(=O)OC)c(NC(=O)c3ccc(-n4ccnc4)nn3)c2F)c(F)c1NC(=O)c1ccc(-n2ccnc2)nn1] |
| 37 | 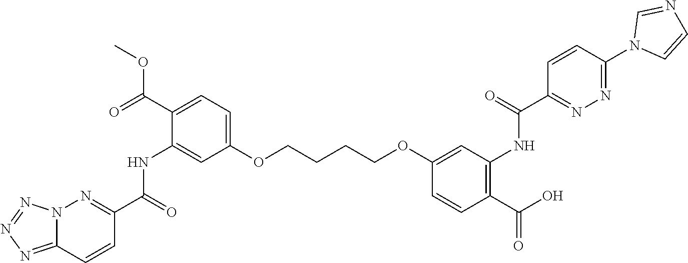 [SMILES: COC(=O)c1ccc(OCCCCOc2ccc(C(=O)O)c(NC(=O)c3ccc(-n4ccnc4)nn3)c2)cc1NC(=O)c1ccc2nnnn2n1] |
| 38 | 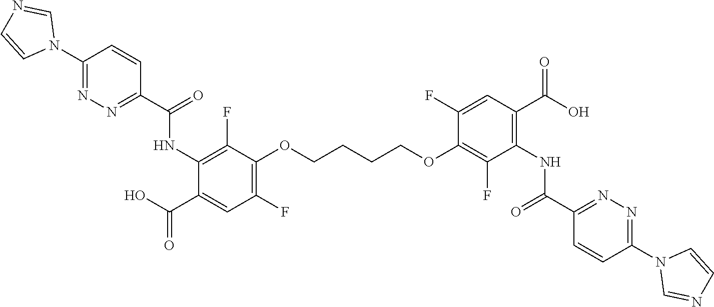 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(-n2ccnc2)nn1] |
| 39 | 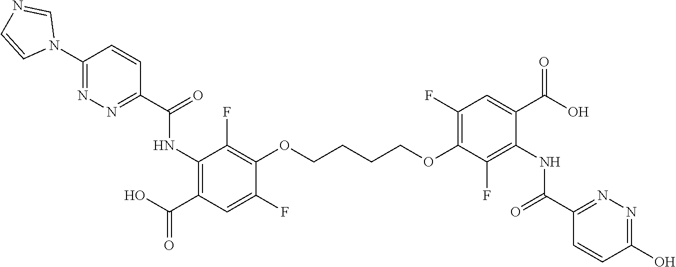 [SMILES: O=C(Nc1c(C(=O)O)cc(F)c(OCCCCOc2c(F)cc(C(=O)O)c(NC(=O)c3ccc(-n4ccnc4)nn3)c2F)c1F)c1ccc(O)nn1] |
| 40 | 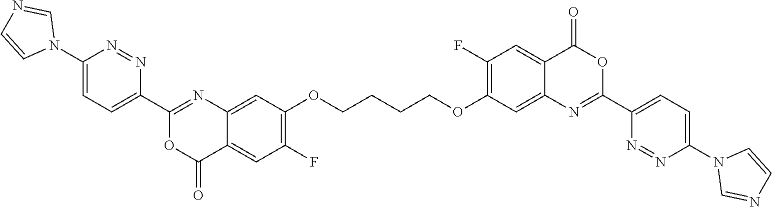 [SMILES: O=c1oc(-c2ccc(-n3ccnc3)nn2)nc2cc(OCCCCOc3cc4nc(-c5ccc(-n6ccnc6)nn5)oc(=O)c4cc3F)c(F)cc12] |
| 41 | 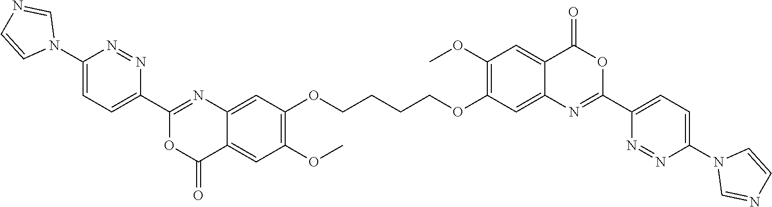 [SMILES: COc1cc2c(=O)oc(-c3ccc(-n4ccnc4)nn3)nc2cc1OCCCCOc1cc2nc(-c3ccc(-n4ccnc4)nn3)oc(=O)c2cc1OC] |
| 42 | 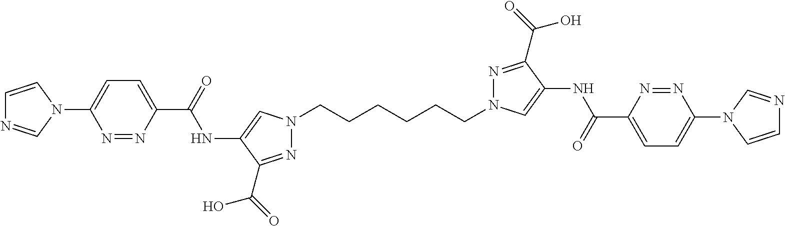 [SMILES: O=C(Nc1cn(CCCCCCn2cc(NC(=O)c3ccc(-n4ccnc4)nn3)c(C(=O)O)n2)nc1C(=O)O)c1ccc(-n2ccnc2)nn1] |
| 43 | 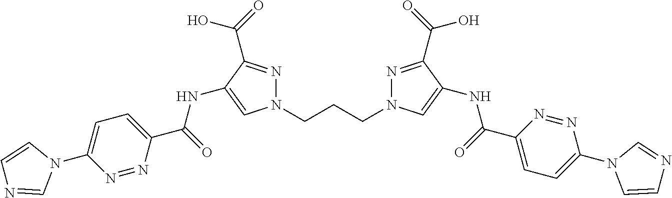 [SMILES: O=C(Nc1cn(CCCn2cc(NC(=O)c3ccc(-n4ccnc4)nn3)c(C(=O)O)n2)nc1C(=O)O)c1ccc(-n2ccnc2)nn1] |
| 44 | 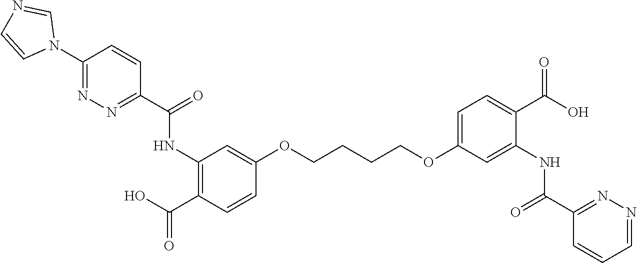 [SMILES: O=C(Nc1cc(OCCCCOc2ccc(C(=O)O)c(NC(=O)c3ccc(-n4ccnc4)nn3)c2)ccc1C(=O)O)c1cccnn1] |
| 45 | 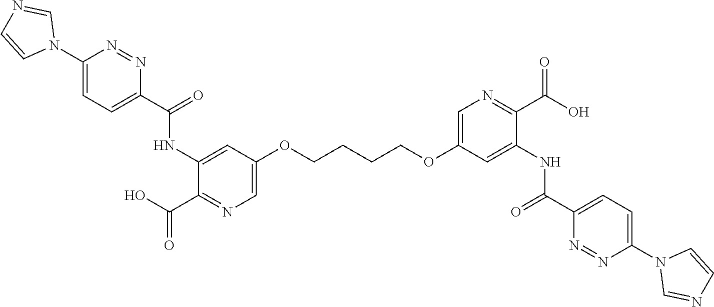 [SMILES: O=C(Nc1cc(OCCCCOc2cnc(C(=O)O)c(NC(=O)c3ccc(-n4ccnc4)nn3)c2)cnc1C(=O)O)c1ccc(-n2ccnc2)nn1] |
| 46 | 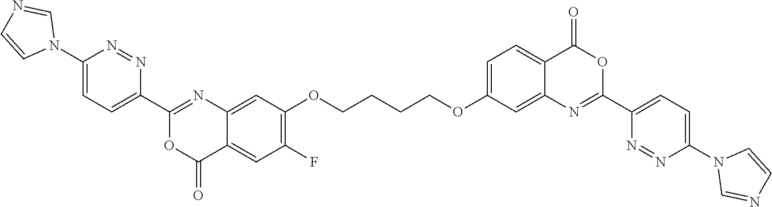 [SMILES: O=c1oc(-c2ccc(-n3ccnc3)nn2)nc2cc(OCCCCOc3cc4nc(-c5ccc(-n6ccnc6)nn5)oc(=O)c4cc3F)ccc12] |
| 47 | 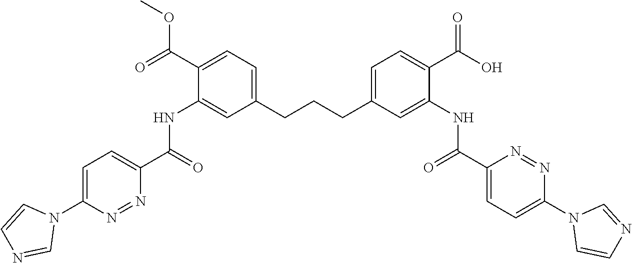 [SMILES: COC(=O)c1ccc(CCCc2ccc(C(=O)O)c(NC(=O)c3ccc(-n4ccnc4)nn3)c2)cc1NC(=O)c1ccc(-n2ccnc2)nn1] |
| 48 | 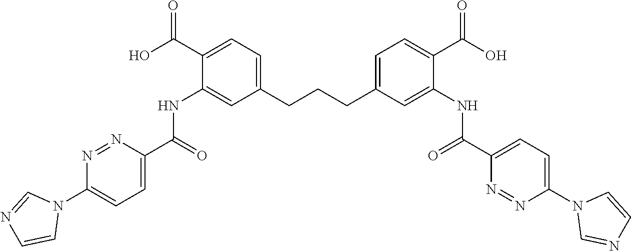 [SMILES: O=C(Nc1cc(CCCc2ccc(C(=O)O)c(NC(=O)c3ccc(-n4ccnc4)nn3)c2)ccc1C(=O)O)c1ccc(-n2ccnc2)nn1] |
| 49 | 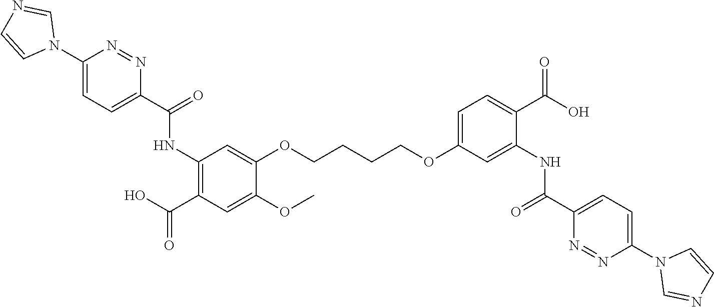 [SMILES: COc1cc(C(=O)O)c(NC(=O)c2ccc(-n3ccnc3)nn2)cc1OCCCCOc1ccc(C(=O)O)c(NC(=O)c2ccc(-n3ccnc3)nn2)c1] |
| 50 | 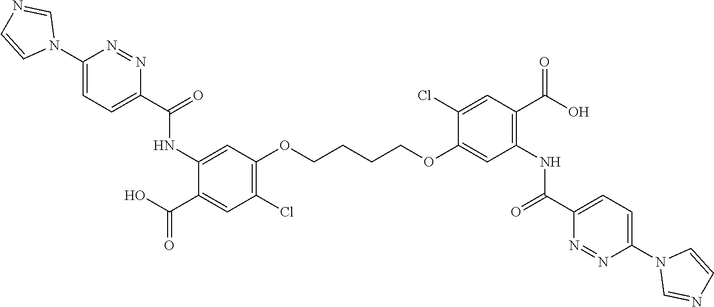 [SMILES: O=C(Nc1cc(OCCCCOc2cc(NC(=O)c3ccc(-n4ccnc4)nn3)c(C(=O)O)cc2Cl)c(Cl)cc1C(=O)O)c1ccc(-n2ccnc2)nn1] |
| 51 | 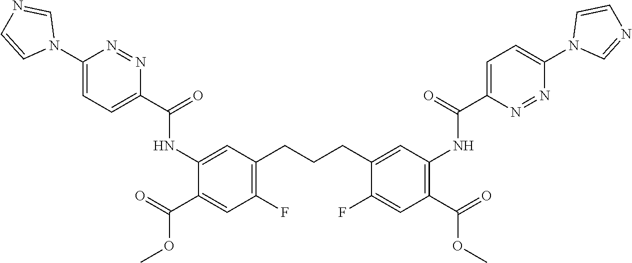 [SMILES: COC(=O)c1cc(F)c(CCCc2cc(NC(=O)c3ccc(-n4ccnc4)nn3)c(C(=O)OC)cc2F)cc1NC(=O)c1ccc(-n2ccnc2)nn1] |
| 52 | 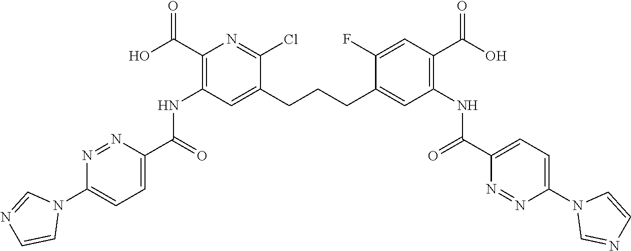 [SMILES: O=C(Nc1cc(CCCc2cc(NC(=O)c3ccc(-n4ccnc4)nn3)c(C(=O)O)nc2Cl)c(F)cc1C(=O)O)c1ccc(-n2ccnc2)nn1] |
| 53 | 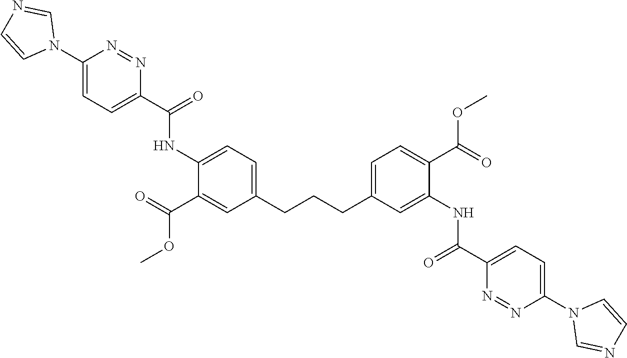 [SMILES: COC(=O)c1ccc(CCCc2ccc(NC(=O)c3ccc(-n4ccnc4)nn3)c(C(=O)OC)c2)cc1NC(=O)c1ccc(-n2ccnc2)nn1] |
| 54 | 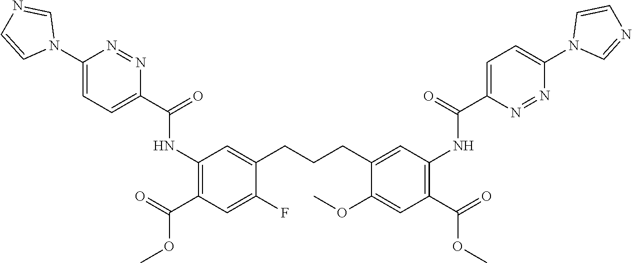 [SMILES: COC(=O)c1cc(F)c(CCCc2cc(NC(=O)c3ccc(-n4ccnc4)nn3)c(C(=O)OC)cc2OC)cc1NC(=O)c1ccc(-n2ccnc2)nn1] |
| 55 | 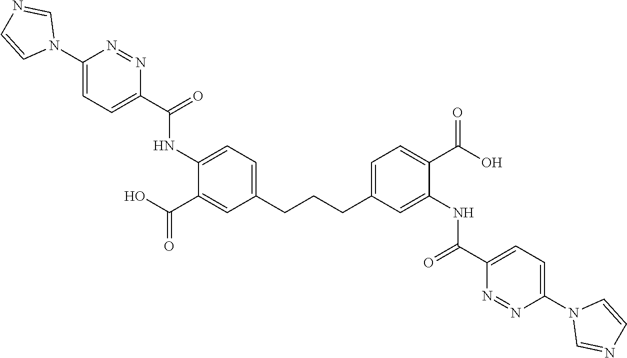 [SMILES: O=C(Nc1cc(CCCc2ccc(NC(=O)c3ccc(-n4ccnc4)nn3)c(C(=O)O)c2)ccc1C(=O)O)c1ccc(-n2ccnc2)nn1] |
| 56 | 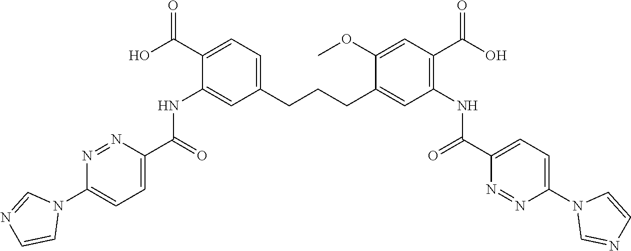 [SMILES: COc1cc(C(=O)O)c(NC(=O)c2ccc(-n3ccnc3)nn2)cc1CCCc1ccc(C(=O)O)c(NC(=O)c2ccc(-n3ccnc3)nn2)c1] |
| 57 | 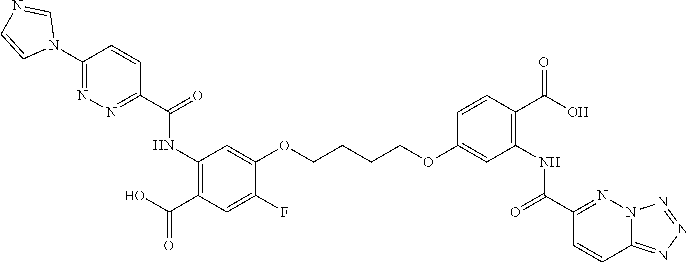 [SMILES: O=C(Nc1cc(OCCCCOc2ccc(C(=O)O)c(NC(=O)c3ccc4nnnn4n3)c2)c(F)cc1C(=O)O)c1ccc(-n2ccnc2)nn1] |
| 58 | 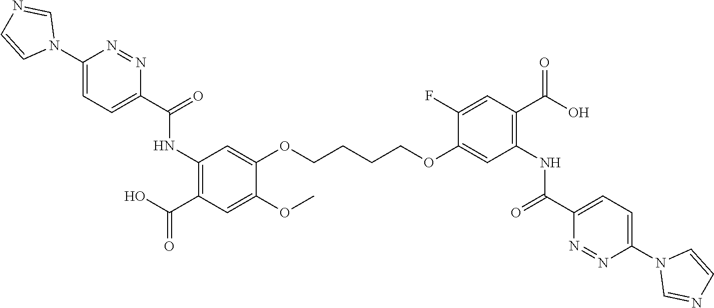 [SMILES: COc1cc(C(=O)O)c(NC(=O)c2ccc(-n3ccnc3)nn2)cc1OCCCCOc1cc(NC(=O)c2ccc(-n3ccnc3)nn2)c(C(=O)O)cc1F] |
| 59 | 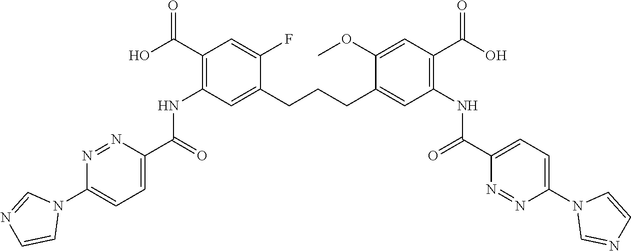 [SMILES: COc1cc(C(=O)O)c(NC(=O)c2ccc(-n3ccnc3)nn2)cc1CCCc1cc(NC(=O)c2ccc(-n3ccnc3)nn2)c(C(=O)O)cc1F] |
| 60 | 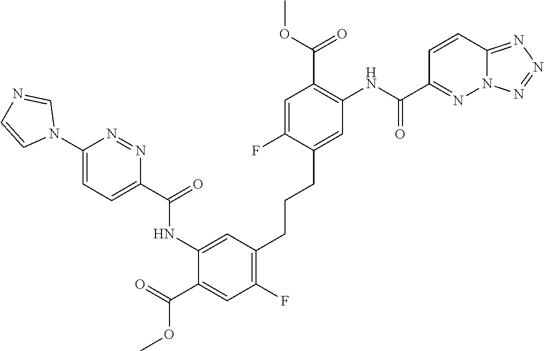 [SMILES: COC(=O)c1cc(F)c(CCCc2cc(NC(=O)c3ccc4nnnn4n3)c(C(=O)OC)cc2F)cc1NC(=O)c1ccc(-n2ccnc2)nn1] |
| 61 | 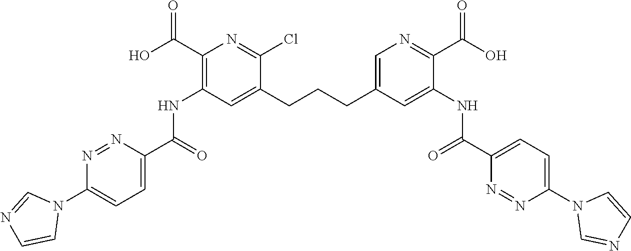 [SMILES: O=C(Nc1cc(CCCc2cc(NC(=O)c3ccc(-n4ccnc4)nn3)c(C(=O)O)nc2Cl)cnc1C(=O)O)c1ccc(-n2ccnc2)nn1] |
| 62 | 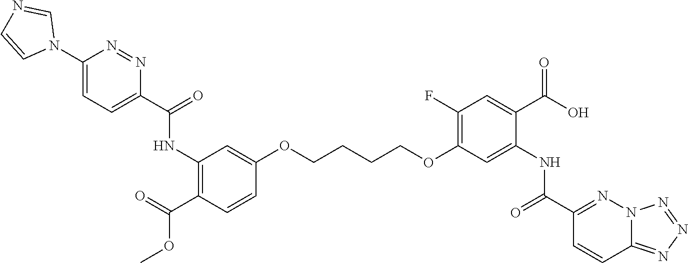 [SMILES: COC(=O)c1ccc(OCCCCOc2cc(NC(=O)c3ccc4nnnn4n3)c(C(=O)O)cc2F)cc1NC(=O)c1ccc(-n2ccnc2)nn1] |
| 63 | 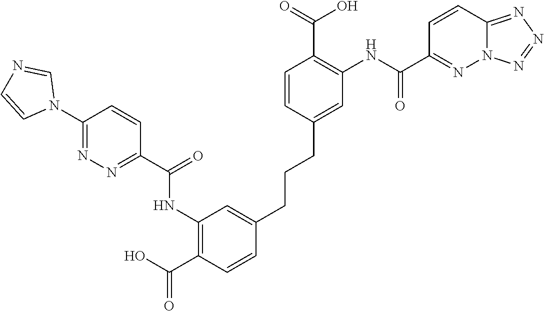 [SMILES: O=C(Nc1cc(CCCc2ccc(C(=O)O)c(NC(=O)c3ccc4nnnn4n3)c2)ccc1C(=O)O)c1ccc(-n2ccnc2)nn1] |
| 64 | 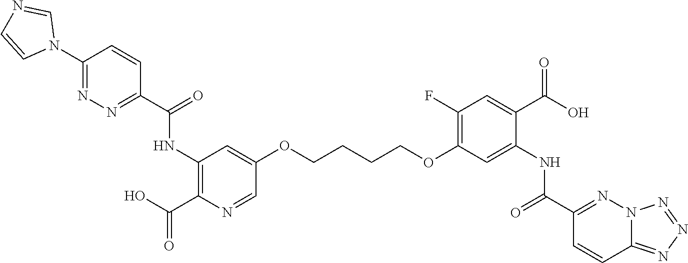 [SMILES: O=C(Nc1cc(OCCCCOc2cnc(C(=O)O)c(NC(=O)c3ccc(-n4ccnc4)nn3)c2)c(F)cc1C(=O)O)c1ccc2nnnn2n1] |
| 65 | 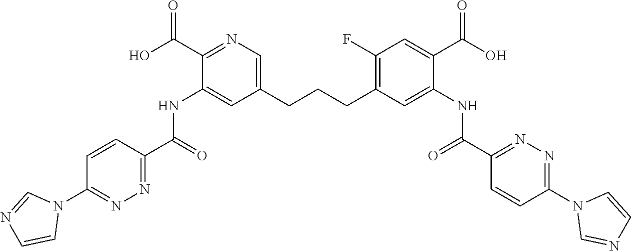 [SMILES: O=C(Nc1cc(CCCc2cnc(C(=O)O)c(NC(=O)c3ccc(-n4ccnc4)nn3)c2)c(F)cc1C(=O)O)c1ccc(-n2ccnc2)nn1] |
| 66 | 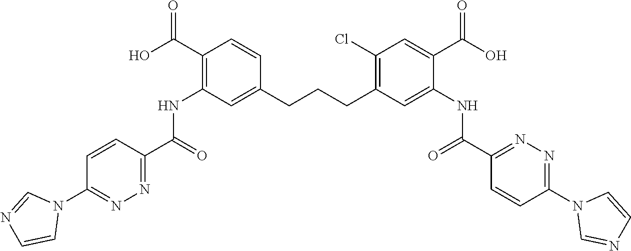 [SMILES: O=C(Nc1cc(CCCc2cc(NC(=O)c3ccc(-n4ccnc4)nn3)c(C(=O)O)cc2Cl)ccc1C(=O)O)c1ccc(-n2ccnc2)nn1] |
| 67 | 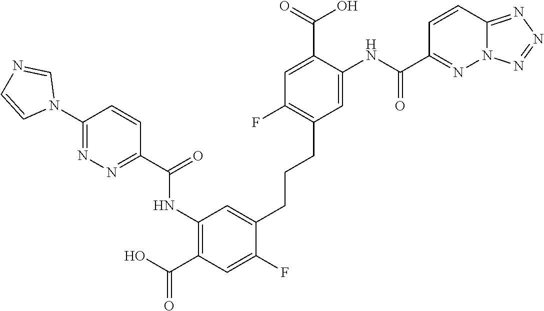 [SMILES: O=C(Nc1cc(CCCc2cc(NC(=O)c3ccc4nnnn4n3)c(C(=O)O)cc2F)c(F)cc1C(=O)O)c1ccc(-n2ccnc2)nn1] |
| 68 | 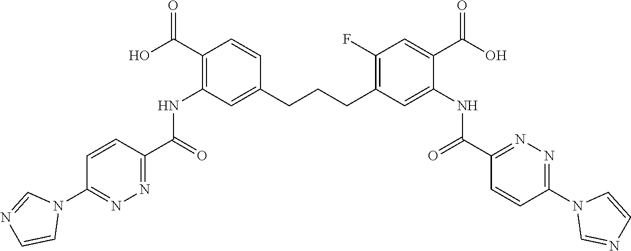 [SMILES: O=C(Nc1cc(CCCc2cc(NC(=O)c3ccc(-n4ccnc4)nn3)c(C(=O)O)cc2F)ccc1C(=O)O)c1ccc(-n2ccnc2)nn1] |
| 69 | 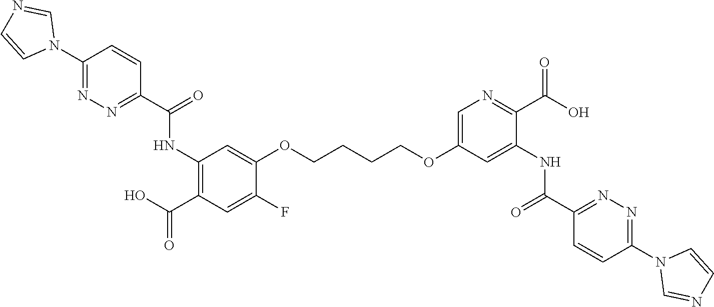 [SMILES: O=C(Nc1cc(OCCCCOc2cnc(C(=O)O)c(NC(=O)c3ccc(-n4ccnc4)nn3)c2)c(F)cc1C(=O)O)c1ccc(-n2ccnc2)nn1] |
| 70 | 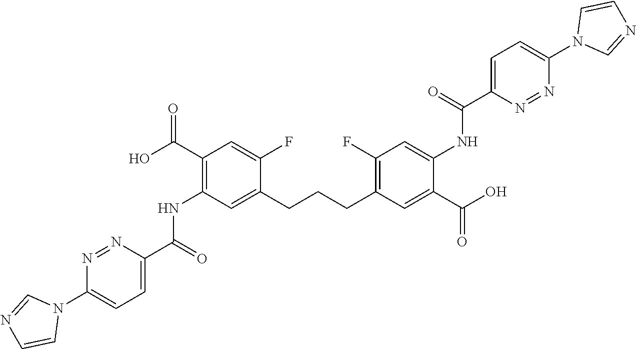 [SMILES: O=C(Nc1cc(CCCc2cc(C(=O)O)c(NC(=O)c3ccc(-n4ccnc4)nn3)cc2F)c(F)cc1C(=O)O)c1ccc(-n2ccnc2)nn1] |
| 71 | 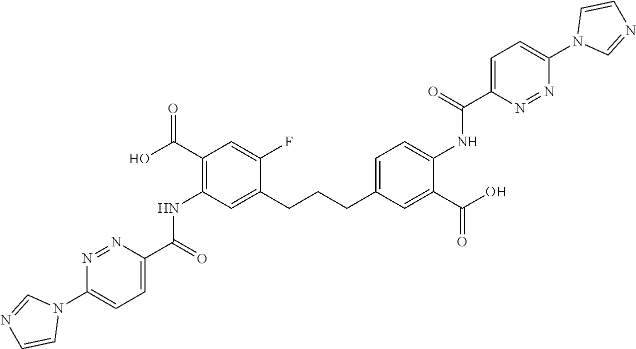 [SMILES: O=C(Nc1ccc(CCCc2cc(NC(=O)c3ccc(-n4ccnc4)nn3)c(C(=O)O)cc2F)cc1C(=O)O)c1ccc(-n2ccnc2)nn1] |
| 72 | 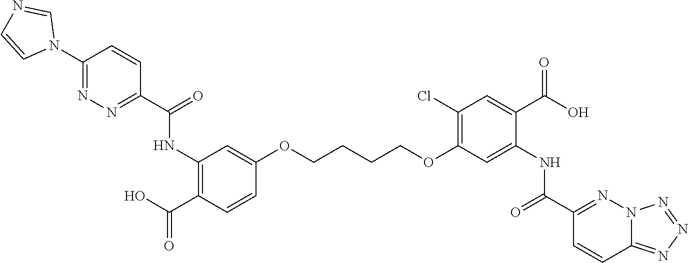 [SMILES: O=C(Nc1cc(OCCCCOc2cc(NC(=O)c3ccc4nnnn4n3)c(C(=O)O)cc2Cl)ccc1C(=O)O)c1ccc(-n2ccnc2)nn1] |
| 73 | 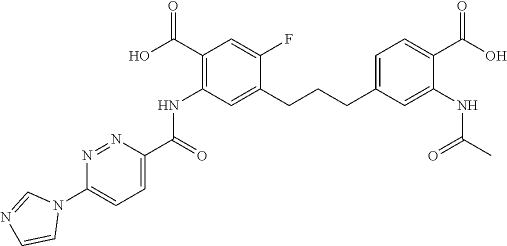 [SMILES: CC(=O)Nc1cc(CCCc2cc(NC(=O)c3ccc(-n4ccnc4)nn3)c(C(=O)O)cc2F)ccc1C(=O)O] |
| 74 | 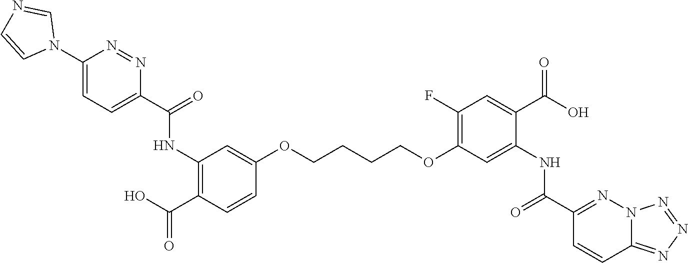 [SMILES: O=C(Nc1cc(OCCCCOc2cc(NC(=O)c3ccc4nnnn4n3)c(C(=O)O)cc2F)ccc1C(=O)O)c1ccc(-n2ccnc2)nn1] |
| 75 | 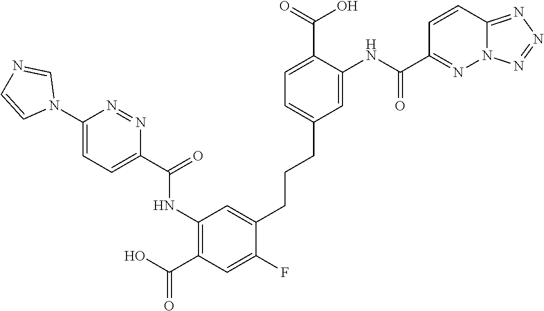 [SMILES: O=C(Nc1cc(CCCc2ccc(C(=O)O)c(NC(=O)c3ccc4nnnn4n3)c2)c(F)cc1C(=O)O)c1ccc(-n2ccnc2)nn1] |
| 76 | 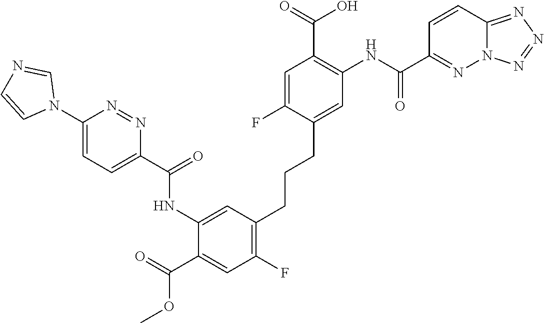 [SMILES: COC(=O)c1cc(F)c(CCCc2cc(NC(=O)c3ccc4nnnn4n3)c(C(=O)O)cc2F)cc1NC(=O)c1ccc(-n2ccnc2)nn1] |
| 77 | 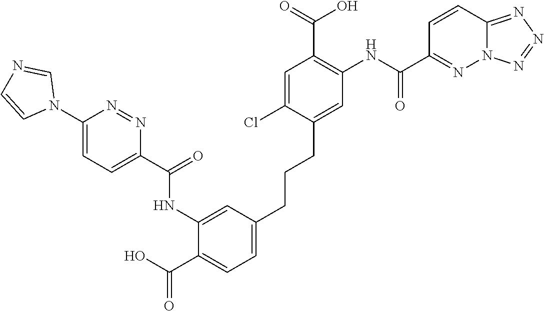 [SMILES: O=C(Nc1cc(CCCc2cc(NC(=O)c3ccc4nnnn4n3)c(C(=O)O)cc2Cl)ccc1C(=O)O)c1ccc(-n2ccnc2)nn1] |
| 78 | 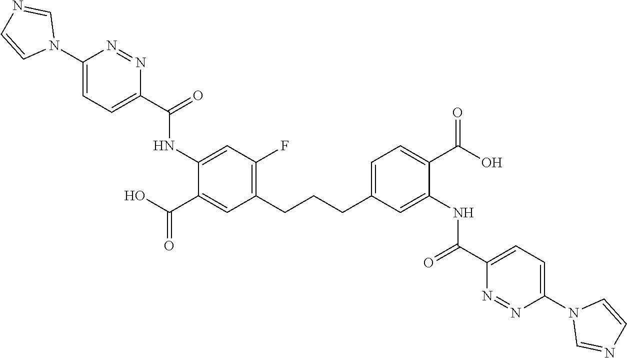 [SMILES: O=C(Nc1cc(CCCc2cc(C(=O)O)c(NC(=O)c3ccc(-n4ccnc4)nn3)cc2F)ccc1C(=O)O)c1ccc(-n2ccnc2)nn1] |
| 79 | 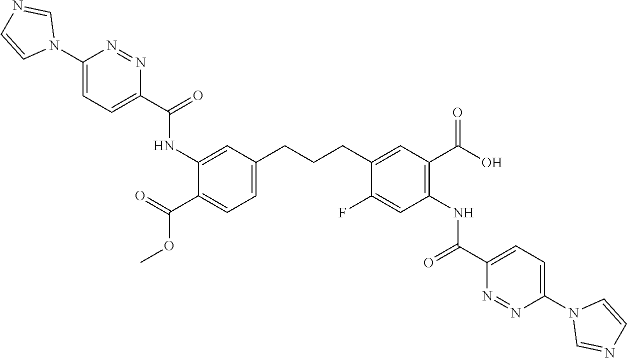 [SMILES: COC(=O)c1ccc(CCCc2cc(C(=O)O)c(NC(=O)c3ccc(-n4ccnc4)nn3)cc2F)cc1NC(=O)c1ccc(-n2ccnc2)nn1] |
| 80 | 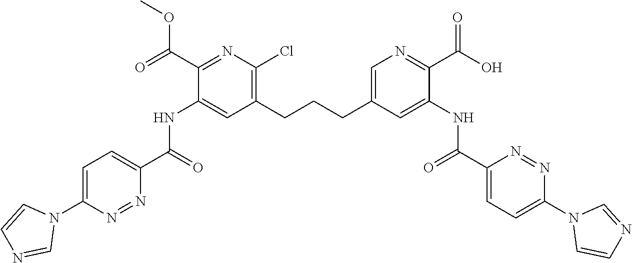 [SMILES: COC(=O)c1nc(Cl)c(CCCc2cnc(C(=O)O)c(NC(=O)c3ccc(-n4ccnc4)nn3)c2)cc1NC(=O)c1ccc(-n2ccnc2)nn1] |
| 81 | 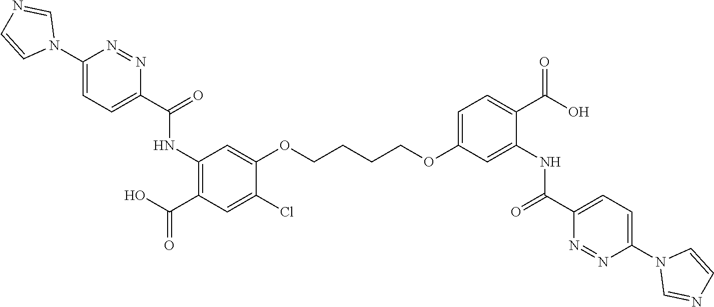 [SMILES: O=C(Nc1cc(OCCCCOc2cc(NC(=O)c3ccc(-n4ccnc4)nn3)c(C(=O)O)cc2Cl)ccc1C(=O)O)c1ccc(-n2ccnc2)nn1] |
| 82 | 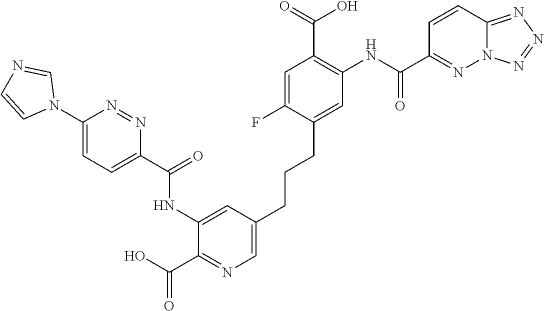 [SMILES: O=C(Nc1cc(CCCc2cnc(C(=O)O)c(NC(=O)c3ccc(-n4ccnc4)nn3)c2)c(F)cc1C(=O)O)c1ccc2nnnn2n1] |
| 83 | 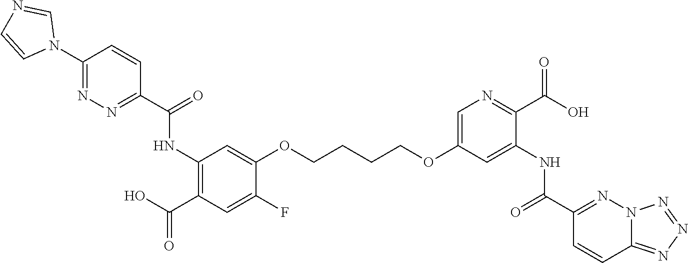 [SMILES: O=C(Nc1cc(OCCCCOc2cnc(C(=O)O)c(NC(=O)c3ccc4nnnn4n3)c2)c(F)cc1C(=O)O)c1ccc(-n2ccnc2)nn1] |
| 84 | 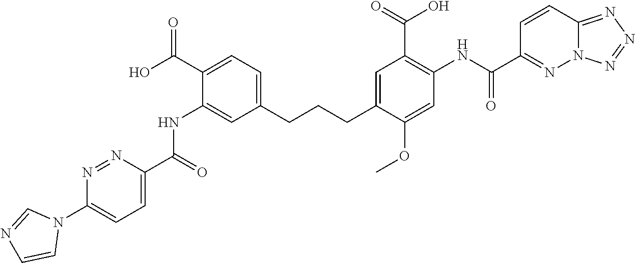 [SMILES: COc1cc(NC(=O)c2ccc3nnnn3n2)c(C(=O)O)cc1CCCc1ccc(C(=O)O)c(NC(=O)c2ccc(-n3ccnc3)nn2)c1] |
| 85 | 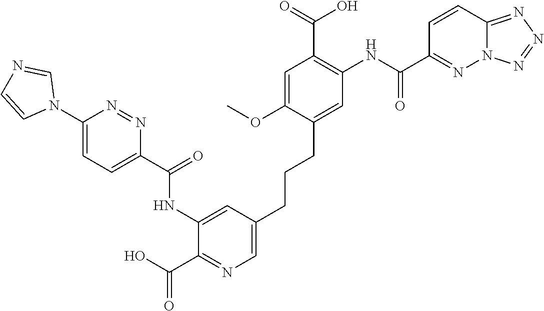 [SMILES: COc1cc(C(=O)O)c(NC(=O)c2ccc3nnnn3n2)cc1CCCc1cnc(C(=O)O)c(NC(=O)c2ccc(-n3ccnc3)nn2)c1] |
| 86 | 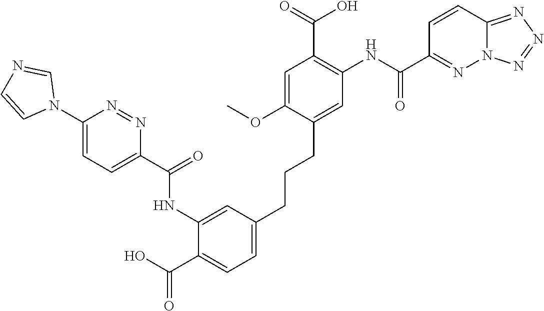 [SMILES: COc1cc(C(=O)O)c(NC(=O)c2ccc3nnnn3n2)cc1CCCc1ccc(C(=O)O)c(NC(=O)c2ccc(-n3ccnc3)nn2)c1] |
| 87 | 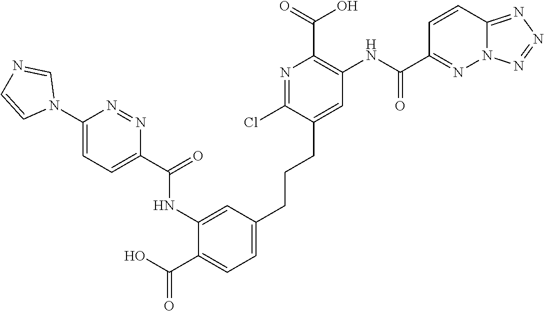 [SMILES: O=C(Nc1cc(CCCc2cc(NC(=O)c3ccc4nnnn4n3)c(C(=O)O)nc2Cl)ccc1C(=O)O)c1ccc(-n2ccnc2)nn1] |
| 88 | 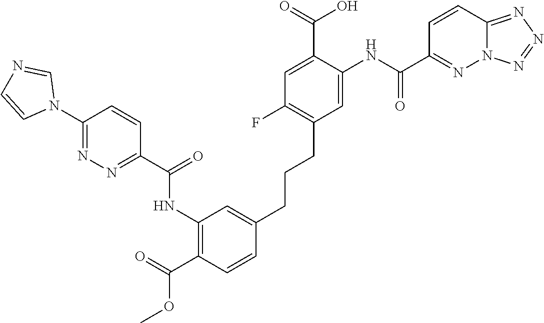 [SMILES: COC(=O)c1ccc(CCCc2cc(NC(=O)c3ccc4nnnn4n3)c(C(=O)O)cc2F)cc1NC(=O)c1ccc(-n2ccnc2)nn1] |
| 89 | 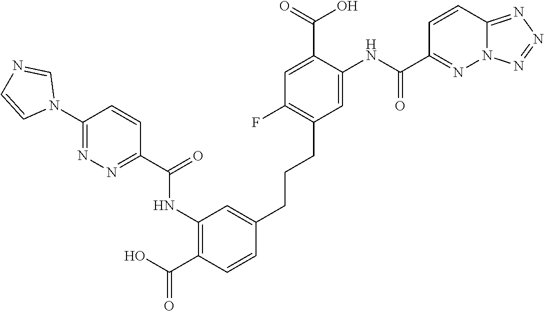 [SMILES: O=C(Nc1cc(CCCc2cc(NC(=O)c3ccc4nnnn4n3)c(C(=O)O)cc2F)ccc1C(=O)O)c1ccc(-n2ccnc2)nn1] |
| 90 | 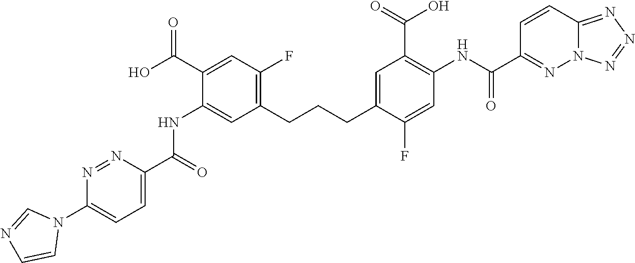 [SMILES: O=C(Nc1cc(CCCc2cc(C(=O)O)c(NC(=O)c3ccc4nnnn4n3)cc2F)c(F)cc1C(=O)O)c1ccc(-n2ccnc2)nn1] |
| 91 | 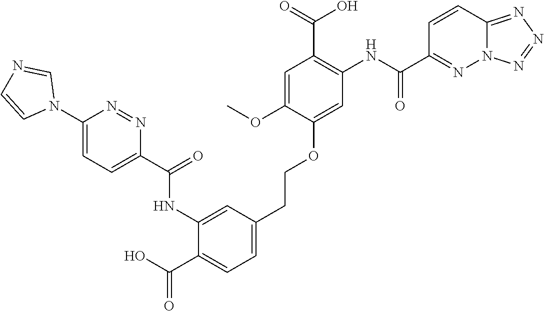 [SMILES: COc1cc(C(=O)O)c(NC(=O)c2ccc3nnnn3n2)cc1OCCc1ccc(C(=O)O)c(NC(=O)c2ccc(-n3ccnc3)nn2)c1] |
| 92 | 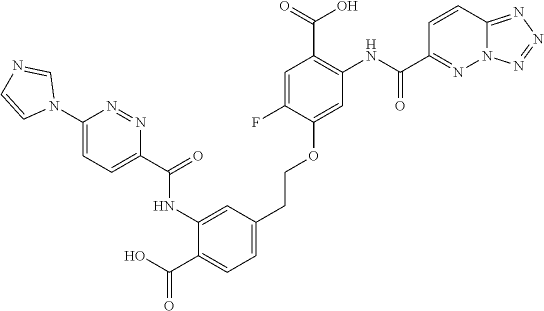 [SMILES: O=C(Nc1cc(CCOc2cc(NC(=O)c3ccc4nnnn4n3)c(C(=O)O)cc2F)ccc1C(=O)O)c1ccc(-n2ccnc2)nn1] |
| 93 | 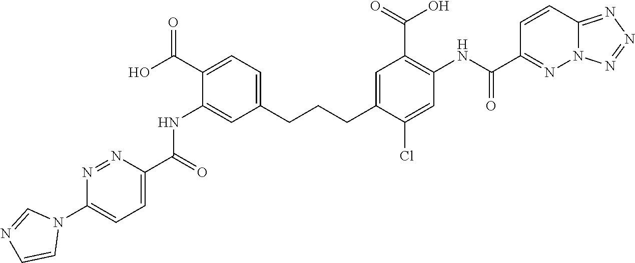 [SMILES: O=C(Nc1cc(CCCc2cc(C(=O)O)c(NC(=O)c3ccc4nnnn4n3)cc2Cl)ccc1C(=O)O)c1ccc(-n2ccnc2)nn1] |
| 94 | 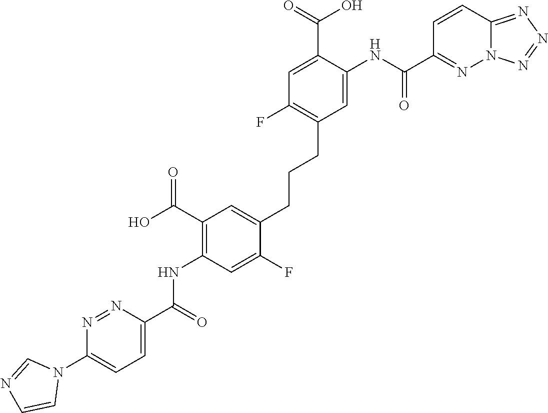 [SMILES: O=C(Nc1cc(F)c(CCCc2cc(NC(=O)c3ccc4nnnn4n3)c(C(=O)O)cc2F)cc1C(=O)O)c1ccc(-n2ccnc2)nn1] |
| 95 | 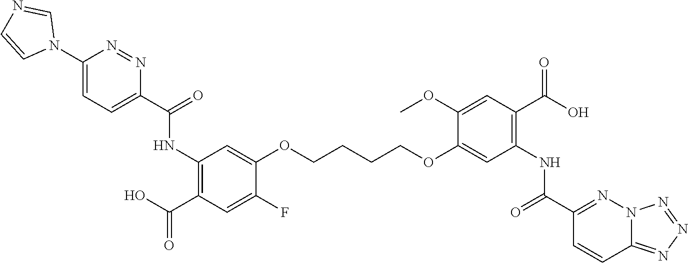 [SMILES: COc1cc(C(=O)O)c(NC(=O)c2ccc3nnnn3n2)cc1OCCCCOc1cc(NC(=O)c2ccc(-n3ccnc3)nn2)c(C(=O)O)cc1F] |
| 96 | 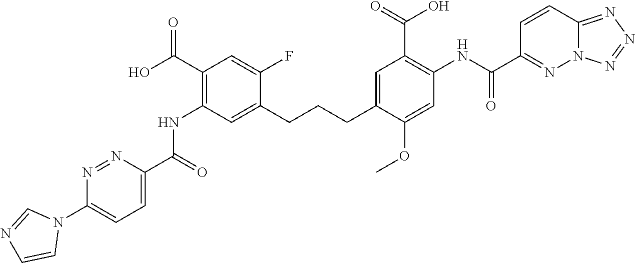 [SMILES: COc1cc(NC(=O)c2ccc3nnnn3n2)c(C(=O)O)cc1CCCc1cc(NC(=O)c2ccc(-n3ccnc3)nn2)c(C(=O)O)cc1F] |
| 97 | 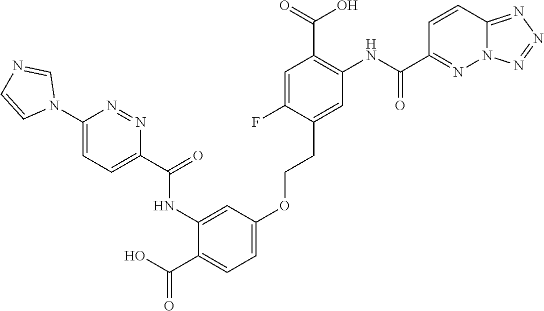 [SMILES: O=C(Nc1cc(OCCc2cc(NC(=O)c3ccc4nnnn4n3)c(C(=O)O)cc2F)ccc1C(=O)O)c1ccc(-n2ccnc2)nn1] |
| 98 | 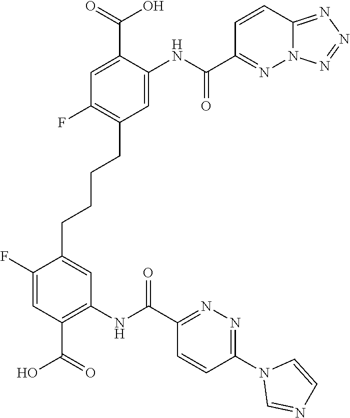 [SMILES: O=C(Nc1cc(CCCCc2cc(NC(=O)c3ccc4nnnn4n3)c(C(=O)O)cc2F)c(F)cc1C(=O)O)c1ccc(-n2ccnc2)nn1] |
| 99 | 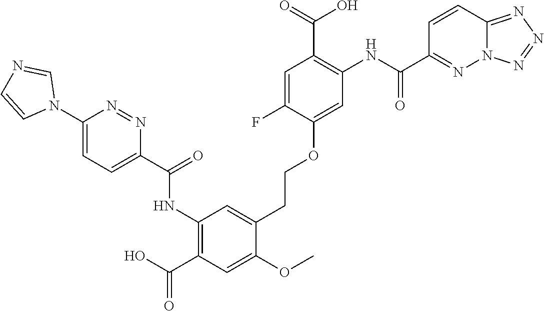 [SMILES: COc1cc(C(=O)O)c(NC(=O)c2ccc(-n3ccnc3)nn2)cc1CCOc1cc(NC(=O)c2ccc3nnnn3n2)c(C(=O)O)cc1F] |
| 100 | 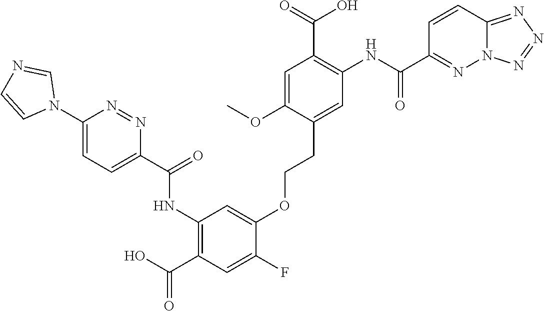 [SMILES: COc1cc(C(=O)O)c(NC(=O)c2ccc3nnnn3n2)cc1CCOc1cc(NC(=O)c2ccc(-n3ccnc3)nn2)c(C(=O)O)cc1F] |
| 101 | 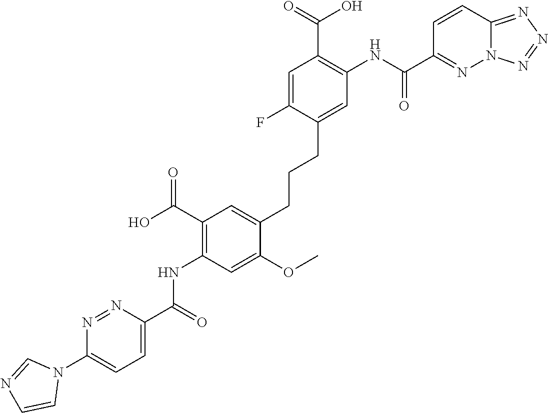 [SMILES: COc1cc(NC(=O)c2ccc(-n3ccnc3)nn2)c(C(=O)O)cc1CCCc1cc(NC(=O)c2ccc3nnnn3n2)c(C(=O)O)cc1F] |
| 102 | 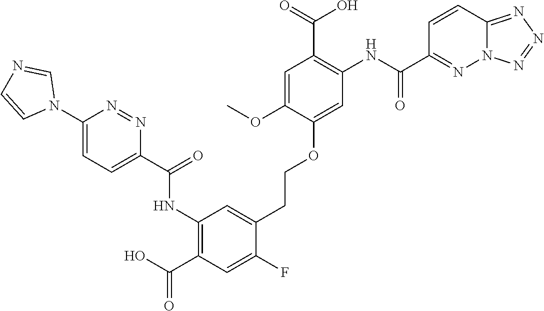 [SMILES: COc1cc(C(=O)O)c(NC(=O)c2ccc3nnnn3n2)cc1OCCc1cc(NC(=O)c2ccc(-n3ccnc3)nn2)c(C(=O)O)cc1F] |
| 103 | 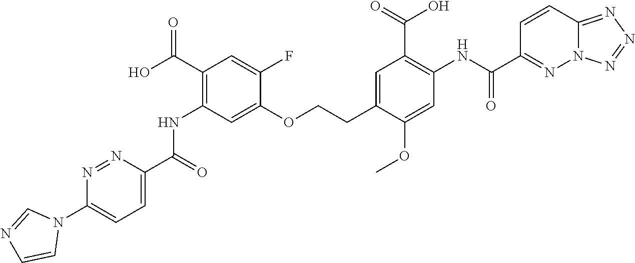 [SMILES: COc1cc(NC(=O)c2ccc3nnnn3n2)c(C(=O)O)cc1CCOc1cc(NC(=O)c2ccc(-n3ccnc3)nn2)c(C(=O)O)cc1F] |
| 104 | 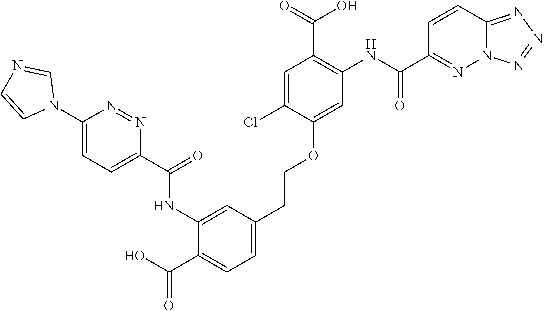 [SMILES: O=C(Nc1cc(CCOc2cc(NC(=O)c3ccc4nnnn4n3)c(C(=O)O)cc2Cl)ccc1C(=O)O)c1ccc(-n2ccnc2)nn1] |
| 105 | 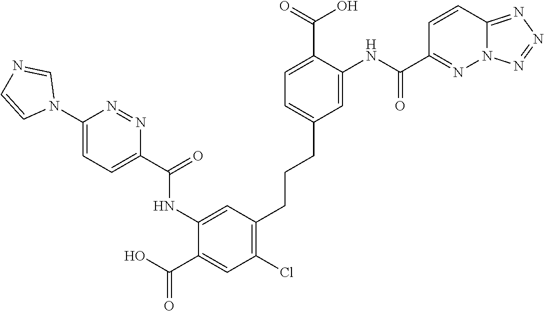 [SMILES: O=C(Nc1cc(CCCc2ccc(C(=O)O)c(NC(=O)c3ccc4nnnn4n3)c2)c(Cl)cc1C(=O)O)c1ccc(-n2ccnc2)nn1] |
| 106 | 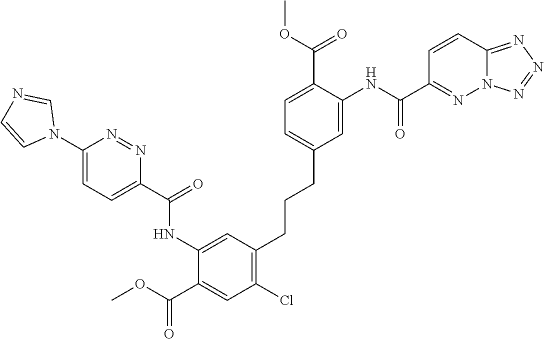 [SMILES: COC(=O)c1cc(Cl)c(CCCc2ccc(C(=O)OC)c(NC(=O)c3ccc4nnnn4n3)c2)cc1NC(=O)c1ccc(-n2ccnc2)nn1] |
| 107 | 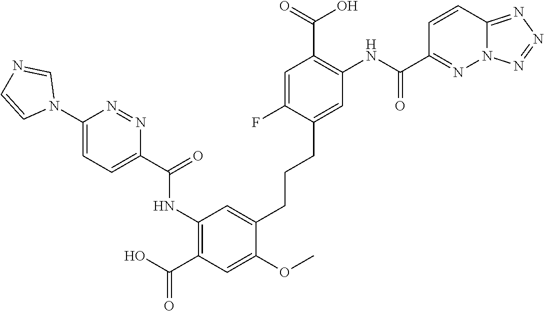 [SMILES: COc1cc(C(=O)O)c(NC(=O)c2ccc(-n3ccnc3)nn2)cc1CCCc1cc(NC(=O)c2ccc3nnnn3n2)c(C(=O)O)cc1F] |
| 108 | 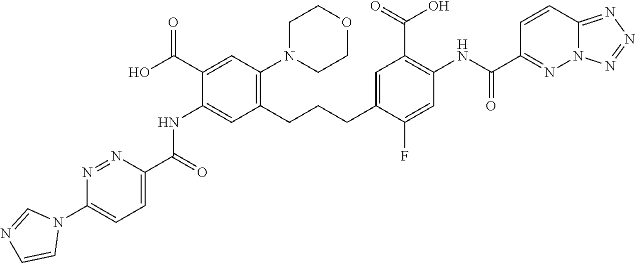 [SMILES: O=C(Nc1cc(CCCc2cc(C(=O)O)c(NC(=O)c3ccc4nnnn4n3)cc2F)c(N2CCOCC2)cc1C(=O)O)c1ccc(-n2ccnc2)nn1] |
| 109 | 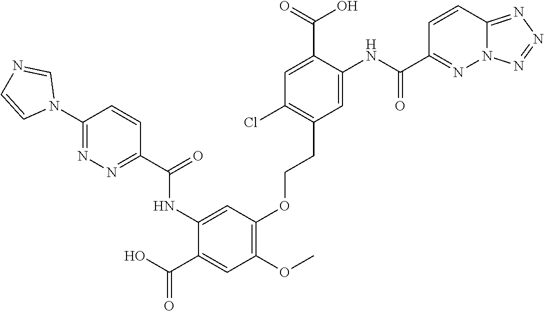 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1Cl] |
| 110 | 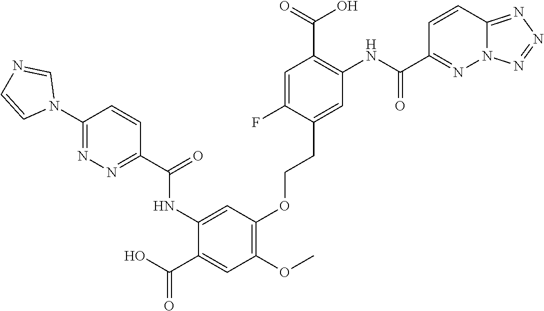 [SMILES: COc1cc(C(=O)O)c(NC(=O)c2ccc(-n3ccnc3)nn2)cc1OCCc1cc(NC(=O)c2ccc3nnnn3n2)c(C(=O)O)cc1F] |
| 111 | 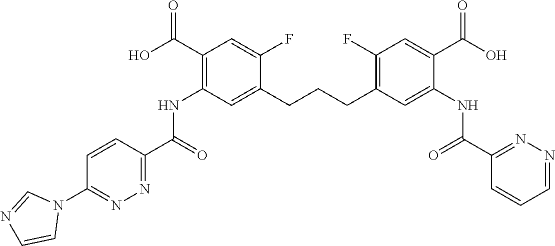 [SMILES: O=C(Nc1cc(CCCc2cc(NC(=O)c3ccc(-n4ccnc4)nn3)c(C(=O)O)cc2F)c(F)cc1C(=O)O)c1cccnn1] |
| 112 | 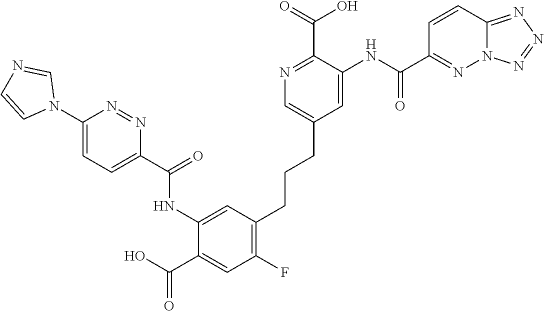 [SMILES: O=C(Nc1cc(CCCc2cnc(C(=O)O)c(NC(=O)c3ccc4nnnn4n3)c2)c(F)cc1C(=O)O)c1ccc(-n2ccnc2)nn1] |
| 113 | 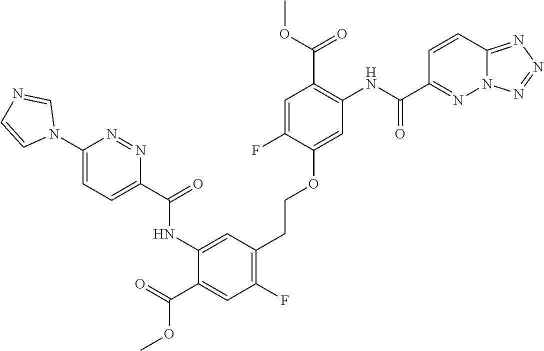 [SMILES: COC(=O)c1cc(F)c(CCOc2cc(NC(=O)c3ccc4nnnn4n3)c(C(=O)OC)cc2F)cc1NC(=O)c1ccc(-n2ccnc2)nn1] |
| 114 | 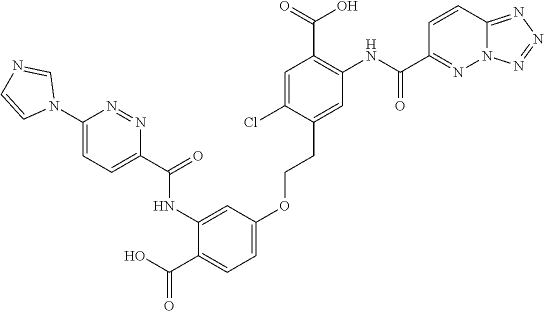 [SMILES: O=C(Nc1cc(OCCc2cc(NC(=O)c3ccc4nnnn4n3)c(C(=O)O)cc2Cl)ccc1C(=O)O)c1ccc(-n2ccnc2)nn1] |
| 115 | 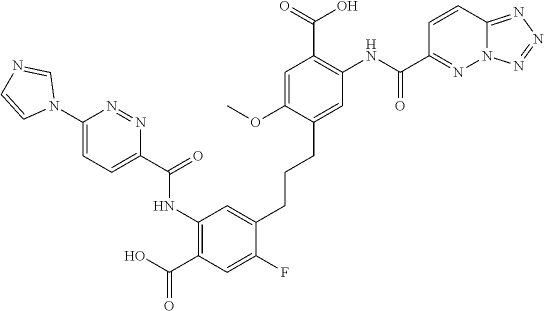 [SMILES: COc1cc(C(=O)O)c(NC(=O)c2ccc3nnnn3n2)cc1CCCc1cc(NC(=O)c2ccc(-n3ccnc3)nn2)c(C(=O)O)cc1F] |
| 116 | 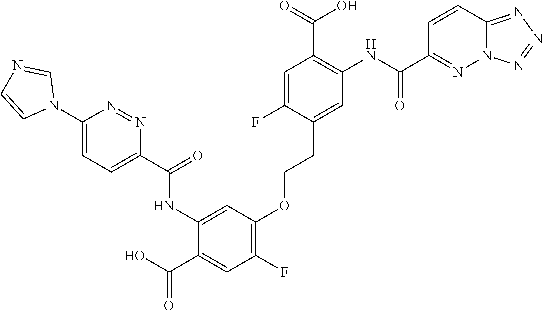 [SMILES: O=C(Nc1cc(OCCc2cc(NC(=O)c3ccc4nnnn4n3)c(C(=O)O)cc2F)c(F)cc1C(=O)O)c1ccc(-n2ccnc2)nn1] |
| 117 | 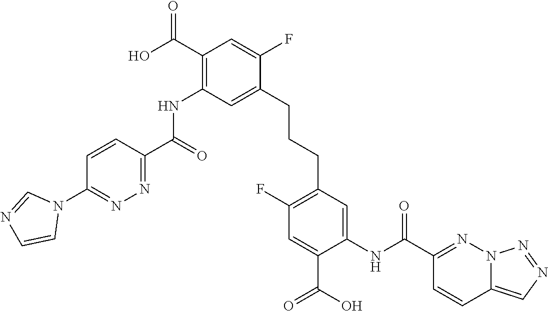 [SMILES: O=C(Nc1cc(CCCc2cc(NC(=O)c3ccc4cnnn4n3)c(C(=O)O)cc2F)c(F)cc1C(=O)O)c1ccc(-n2ccnc2)nn1] |
| 118 | 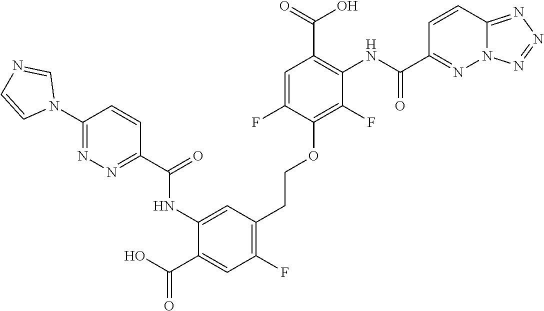 [SMILES: O=C(Nc1cc(CCOc2c(F)cc(C(=O)O)c(NC(=O)c3ccc4nnnn4n3)c2F)c(F)cc1C(=O)O)c1ccc(-n2ccnc2)nn1] |
| 119 | 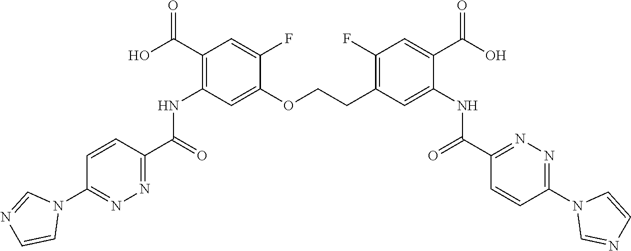 [SMILES: O=C(Nc1cc(CCOc2cc(NC(=O)c3ccc(-n4ccnc4)nn3)c(C(=O)O)cc2F)c(F)cc1C(=O)O)c1ccc(-n2ccnc2)nn1] |
| 120 | 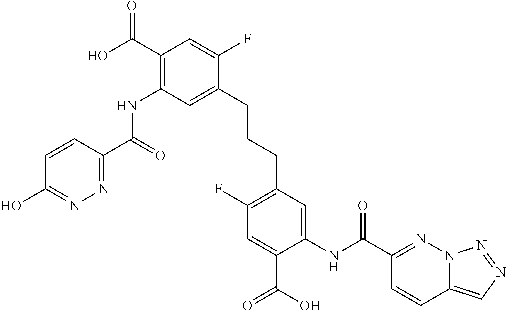 [SMILES: O=C(Nc1cc(CCCc2cc(NC(=O)c3ccc4cnnn4n3)c(C(=O)O)cc2F)c(F)cc1C(=O)O)c1ccc(O)nn1] |
| 121 | 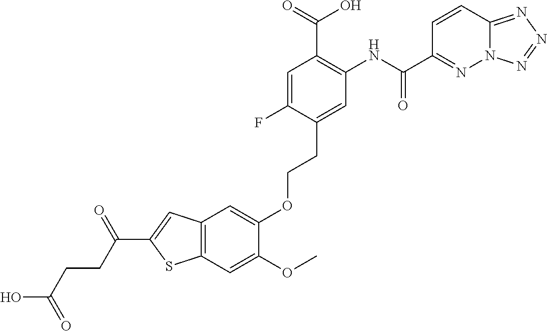 [SMILES: COc1cc2sc(C(=O)CCC(=O)O)cc2cc1OCCc1cc(NC(=O)c2ccc3nnnn3n2)c(C(=O)O)cc1F] |
| 122 | 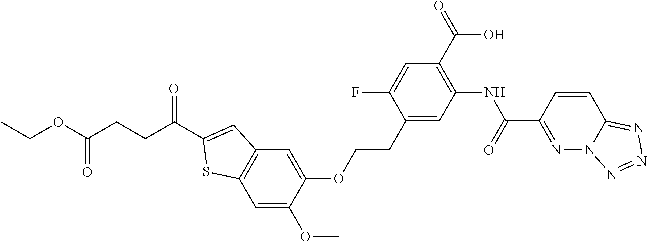 [SMILES: CCOC(=O)CCC(=O)c1cc2cc(OCCc3cc(NC(=O)c4ccc5nnnn5n4)c(C(=O)O)cc3F)c(OC)cc2s1] |
| 123 | 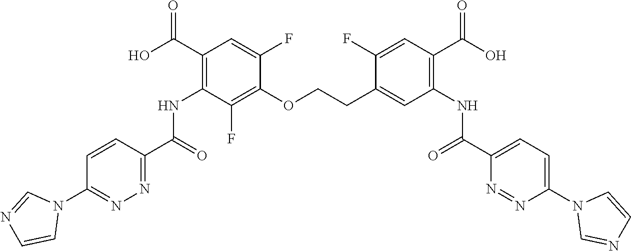 [SMILES: O=C(Nc1cc(CCOc2c(F)cc(C(=O)O)c(NC(=O)c3ccc(-n4ccnc4)nn3)c2F)c(F)cc1C(=O)O)c1ccc(-n2ccnc2)nn1] |
| 124 | 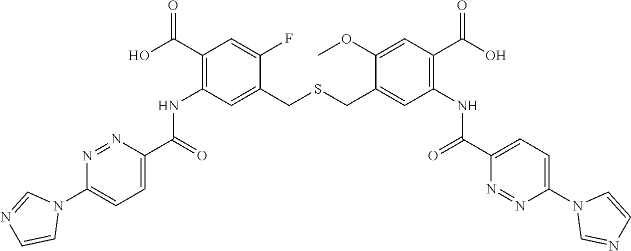 [SMILES: COc1cc(C(=O)O)c(NC(=O)c2ccc(-n3ccnc3)nn2)cc1CSCc1cc(NC(=O)c2ccc(-n3ccnc3)nn2)c(C(=O)O)cc1F] |
| 125 | 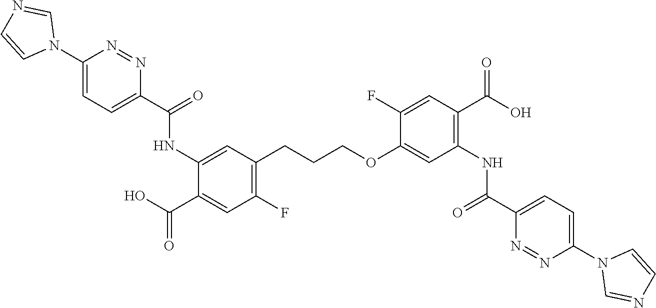 [SMILES: O=C(Nc1cc(CCCOc2cc(NC(=O)c3ccc(-n4ccnc4)nn3)c(C(=O)O)cc2F)c(F)cc1C(=O)O)c1ccc(-n2ccnc2)nn1] |
| 126 | 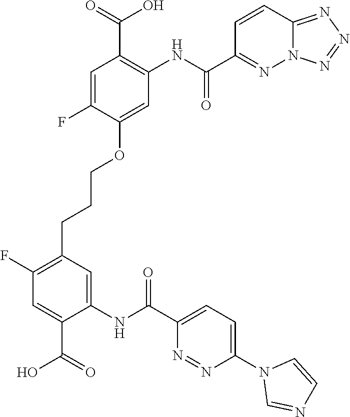 [SMILES: O=C(Nc1cc(CCCOc2cc(NC(=O)c3ccc4nnnn4n3)c(C(=O)O)cc2F)c(F)cc1C(=O)O)c1ccc(-n2ccnc2)nn1] |
| 127 | 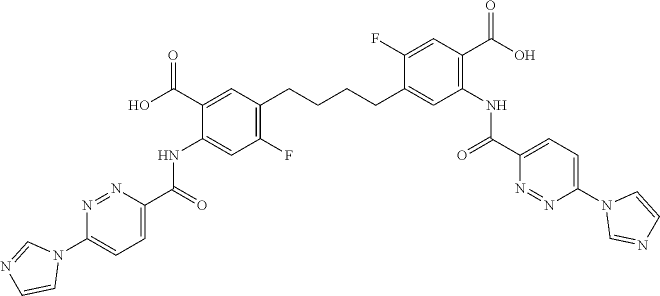 [SMILES: O=C(Nc1cc(CCCCc2cc(C(=O)O)c(NC(=O)c3ccc(-n4ccnc4)nn3)cc2F)c(F)cc1C(=O)O)c1ccc(-n2ccnc2)nn1] |
| 128 | 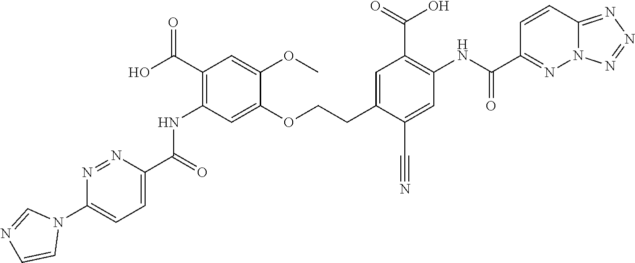 [SMILES: COc1cc(C(=O)O)c(NC(=O)c2ccc(-n3ccnc3)nn2)cc1OCCc1cc(C(=O)O)c(NC(=O)c2ccc3nnnn3n2)cc1C#N] |
| 129 | 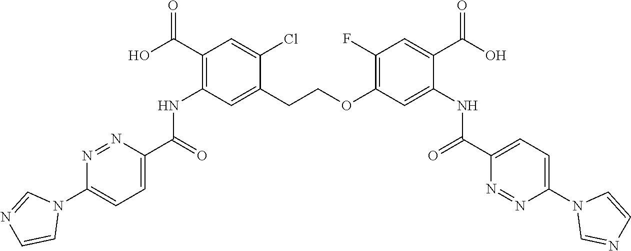 [SMILES: O=C(Nc1cc(OCCc2cc(NC(=O)c3ccc(-n4ccnc4)nn3)c(C(=O)O)cc2Cl)c(F)cc1C(=O)O)c1ccc(-n2ccnc2)nn1] |
| 130 | 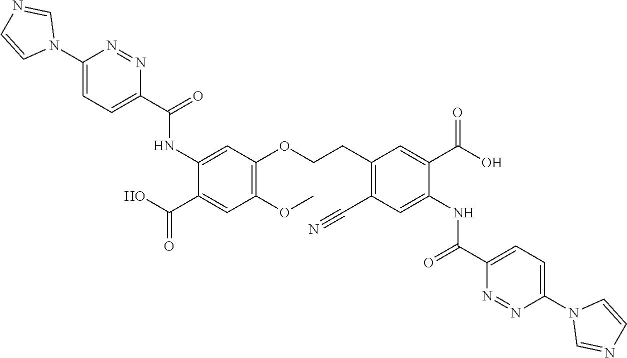 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1C#N] |
| 131 | 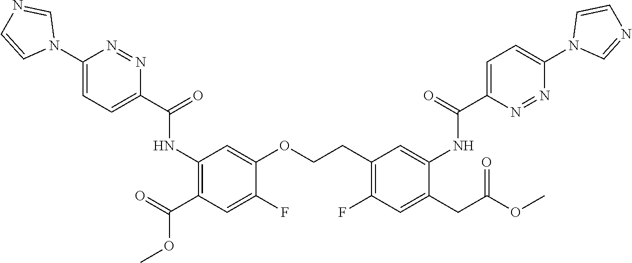 [SMILES: COC(=O)Cc1cc(F)c(CCOc2cc(NC(=O)c3ccc(-n4ccnc4)nn3)c(C(=O)OC)cc2F)cc1NC(=O)c1ccc(-n2ccnc2)nn1] |
| 132 | 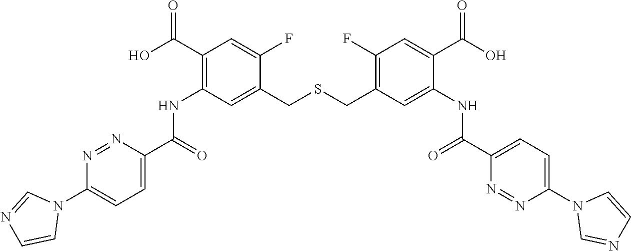 [SMILES: O=C(Nc1cc(CSCc2cc(NC(=O)c3ccc(-n4ccnc4)nn3)c(C(=O)O)cc2F)c(F)cc1C(=O)O)c1ccc(-n2ccnc2)nn1] |
| 133 | 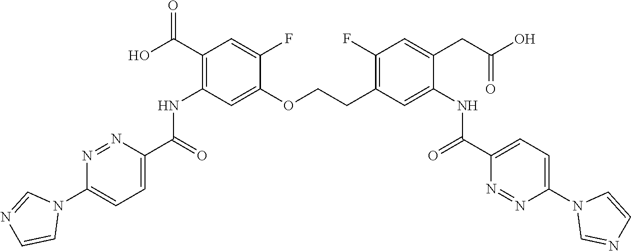 [SMILES: O=C(O)Cc1cc(F)c(CCOc2cc(NC(=O)c3ccc(-n4ccnc4)nn3)c(C(=O)O)cc2F)cc1NC(=O)c1ccc(-n2ccnc2)nn1] |
| 134 | 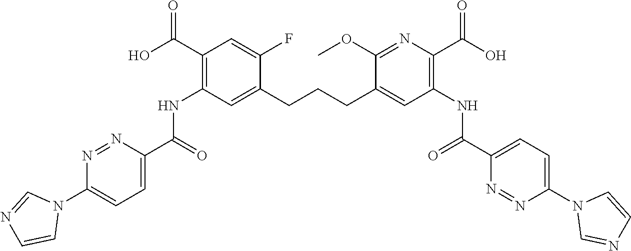 [SMILES: COc1nc(C(=O)O)c(NC(=O)c2ccc(-n3ccnc3)nn2)cc1CCCc1cc(NC(=O)c2ccc(-n3ccnc3)nn2)c(C(=O)O)cc1F] |
| 135 | 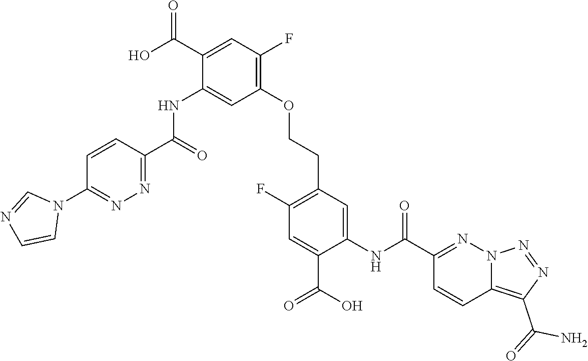 [SMILES: NC(=O)c1nnn2nc(C(=O)Nc3cc(CCOc4cc(NC(=O)c5ccc(-n6ccnc6)nn5)c(C(=O)O)cc4F)c(F)cc3C(=O)O)ccc12] |
| 136 | 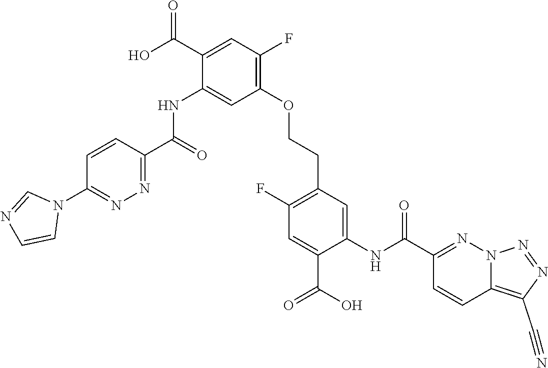 [SMILES: N#Cc1nnn2nc(C(=O)Nc3cc(CCOc4cc(NC(=O)c5ccc(-n6ccnc6)nn5)c(C(=O)O)cc4F)c(F)cc3C(=O)O)ccc12] |
| 137 | 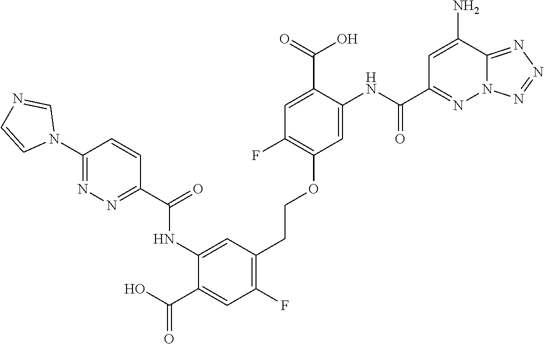 [SMILES: Nc1cc(C(=O)Nc2cc(OCCc3cc(NC(=O)c4ccc(-n5ccnc5)nn4)c(C(=O)O)cc3F)c(F)cc2C(=O)O)nn2nnnc12] |
| 138 | 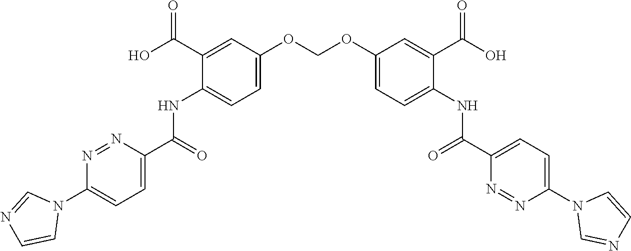 [SMILES: O=C(Nc1ccc(OCOc2ccc(NC(=O)c3ccc(-n4ccnc4)nn3)c(C(=O)O)c2)cc1C(=O)O)c1ccc(-n2ccnc2)nn1] |
| 139 | 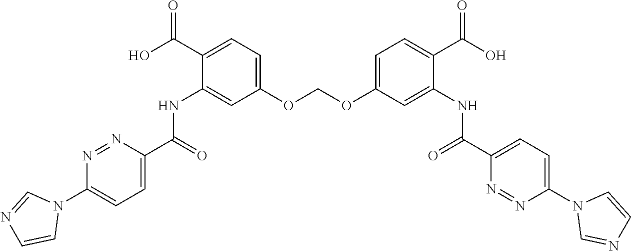 [SMILES: O=C(Nc1cc(OCOc2ccc(C(=O)O)c(NC(=O)c3ccc(-n4ccnc4)nn3)c2)ccc1C(=O)O)c1ccc(-n2ccnc2)nn1] |
| 140 | 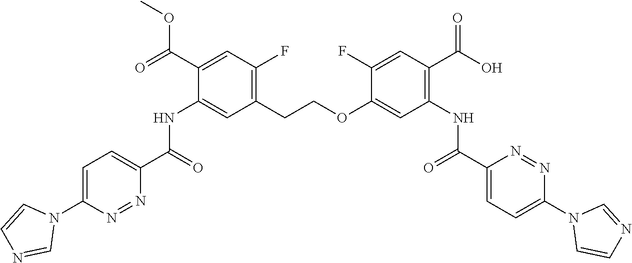 [SMILES: COC(=O)c1cc(F)c(CCOc2cc(NC(=O)c3ccc(-n4ccnc4)nn3)c(C(=O)O)cc2F)cc1NC(=O)c1ccc(-n2ccnc2)nn1] |
| 141 | 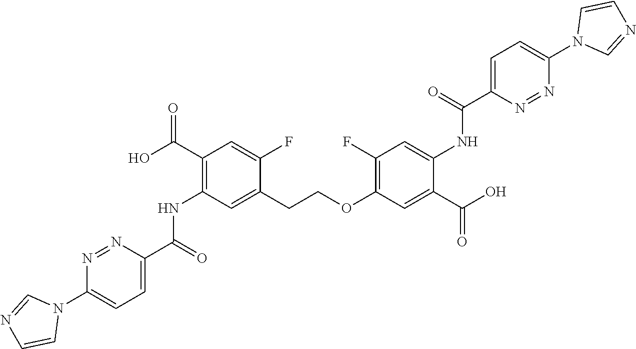 [SMILES: O=C(Nc1cc(CCOc2cc(C(=O)O)c(NC(=O)c3ccc(-n4ccnc4)nn3)cc2F)c(F)cc1C(=O)O)c1ccc(-n2ccnc2)nn1] |
| 142 | 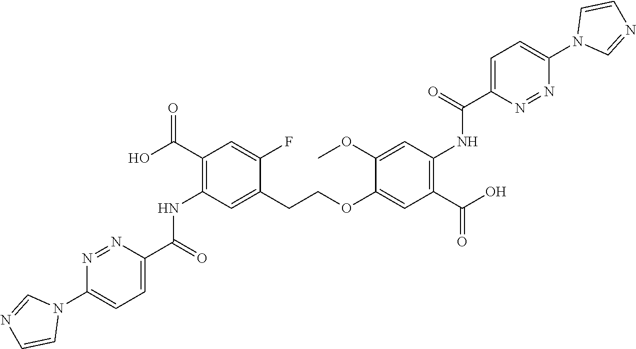 [SMILES: COc1cc(NC(=O)c2ccc(-n3ccnc3)nn2)c(C(=O)O)cc1OCCc1cc(NC(=O)c2ccc(-n3ccnc3)nn2)c(C(=O)O)cc1F] |
| 143 | 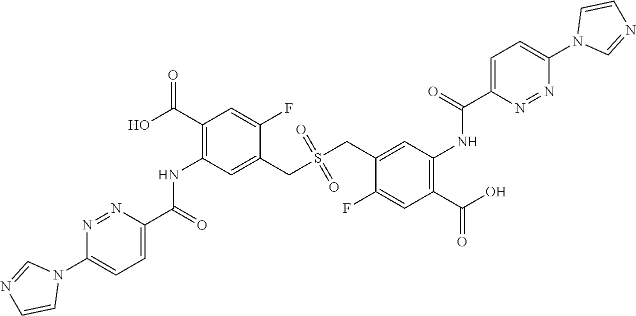 [SMILES: O=C(Nc1cc(CS(=O)(=O)Cc2cc(NC(=O)c3ccc(-n4ccnc4)nn3)c(C(=O)O)cc2F)c(F)cc1C(=O)O)c1ccc(-n2ccnc2)nn1] |
| 144 | 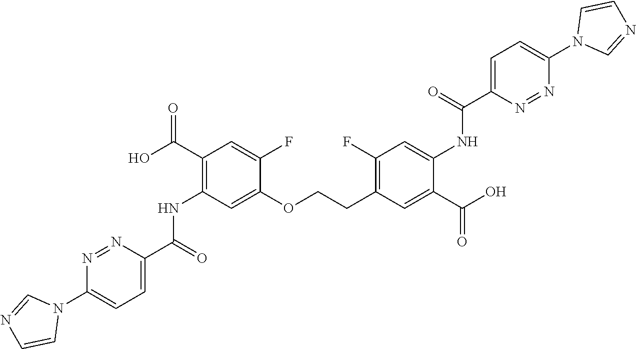 [SMILES: O=C(Nc1cc(OCCc2cc(C(=O)O)c(NC(=O)c3ccc(-n4ccnc4)nn3)cc2F)c(F)cc1C(=O)O)c1ccc(-n2ccnc2)nn1] |
| 145 | 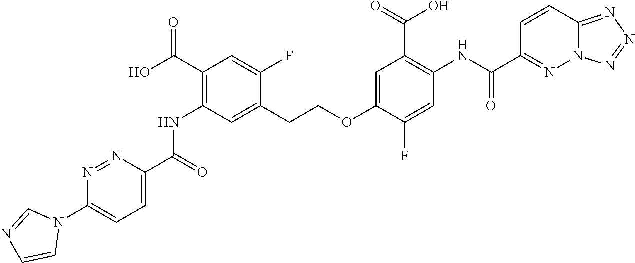 [SMILES: O=C(Nc1cc(CCOc2cc(C(=O)O)c(NC(=O)c3ccc4nnnn4n3)cc2F)c(F)cc1C(=O)O)c1ccc(-n2ccnc2)nn1] |
| 146 | 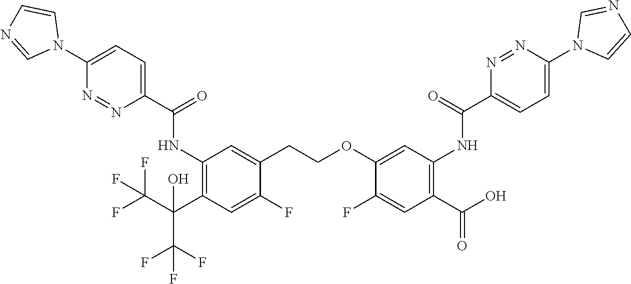 [SMILES: O=C(Nc1cc(OCCc2cc(NC(=O)c3ccc(-n4ccnc4)nn3)c(C(O)(C(F)(F)F)C(F)(F)F)cc2F)c(F)cc1C(=O)O)c1ccc(-n2ccnc2)nn1] |
| 147 | 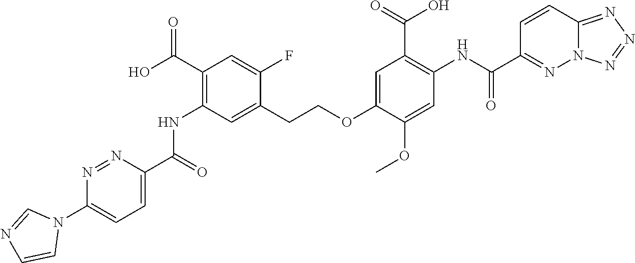 [SMILES: COc1cc(NC(=O)c2ccc3nnnn3n2)c(C(=O)O)cc1OCCc1cc(NC(=O)c2ccc(-n3ccnc3)nn2)c(C(=O)O)cc1F] |
| 148 | 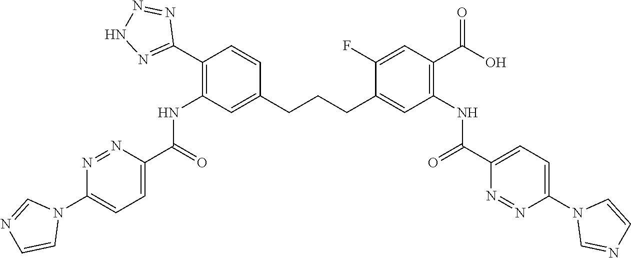 [SMILES: O=C(Nc1cc(CCCc2ccc(-c3nn[nH]n3)c(NC(=O)c3ccc(-n4ccnc4)nn3)c2)c(F)cc1C(=O)O)c1ccc(-n2ccnc2)nn1] |
| 149 | 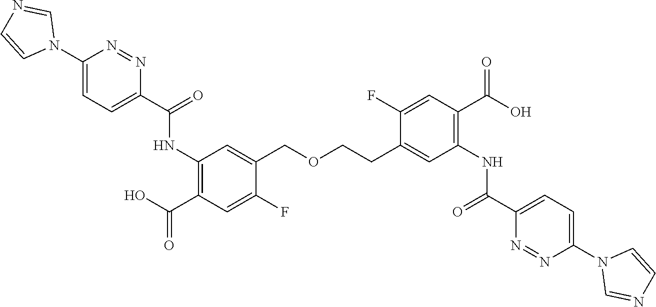 [SMILES: O=C(Nc1cc(CCOCc2cc(NC(=O)c3ccc(-n4ccnc4)nn3)c(C(=O)O)cc2F)c(F)cc1C(=O)O)c1ccc(-n2ccnc2)nn1] |
| 150 | 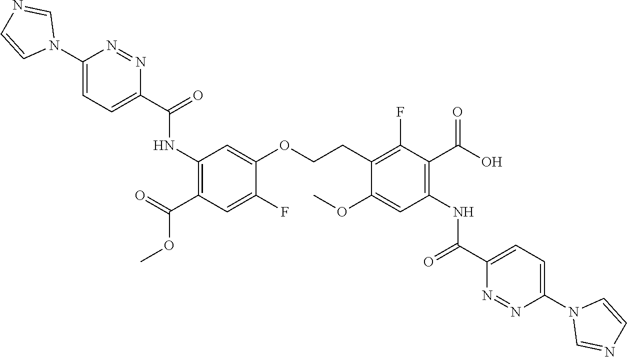 [SMILES: COC(=O)c1cc(F)c(OCCc2c(OC)cc(NC(=O)c3ccc(-n4ccnc4)nn3)c(C(=O)O)c2F)cc1NC(=O)c1ccc(-n2ccnc2)nn1] |
| 151 | 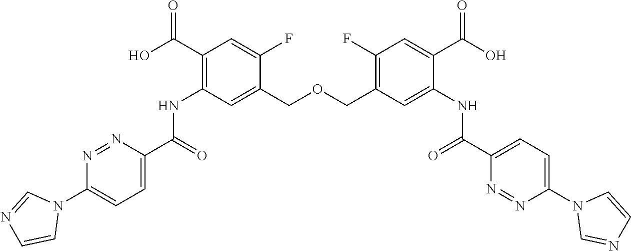 [SMILES: O=C(Nc1cc(COCc2cc(NC(=O)c3ccc(-n4ccnc4)nn3)c(C(=O)O)cc2F)c(F)cc1C(=O)O)c1ccc(-n2ccnc2)nn1] |
| 152 | 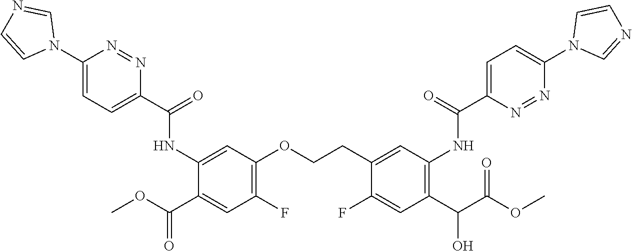 [SMILES: COC(=O)c1cc(F)c(OCCc2cc(NC(=O)c3ccc(-n4ccnc4)nn3)c(C(O)C(=O)OC)cc2F)cc1NC(=O)c1ccc(-n2ccnc2)nn1] |
| 153 | 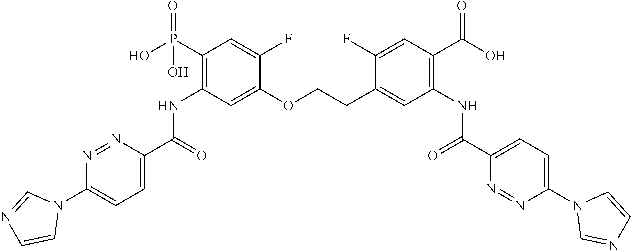 [SMILES: O=C(Nc1cc(CCOc2cc(NC(=O)c3ccc(-n4ccnc4)nn3)c(P(=O)(O)O)cc2F)c(F)cc1C(=O)O)c1ccc(-n2ccnc2)nn1] |
| 154 | 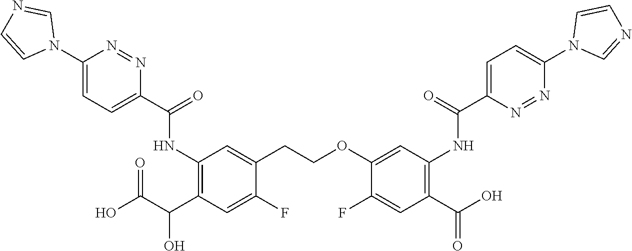 [SMILES: O=C(Nc1cc(OCCc2cc(NC(=O)c3ccc(-n4ccnc4)nn3)c(C(O)C(=O)O)cc2F)c(F)cc1C(=O)O)c1ccc(-n2ccnc2)nn1] |
| 155 | 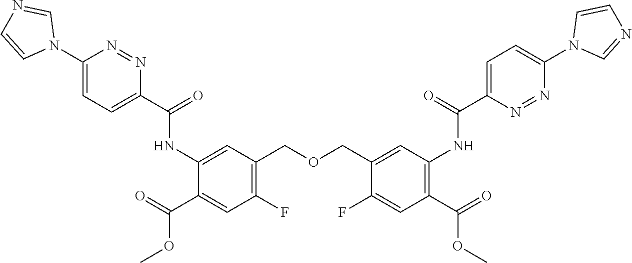 [SMILES: COC(=O)c1cc(F)c(COCc2cc(NC(=O)c3ccc(-n4ccnc4)nn3)c(C(=O)OC)cc2F)cc1NC(=O)c1ccc(-n2ccnc2)nn1] |
| 156 | 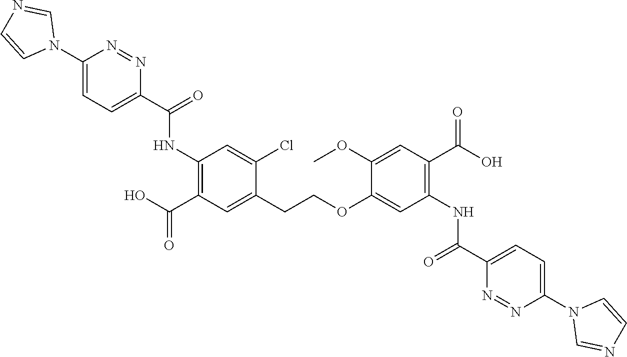 [SMILES: COc1cc(C(=O)O)c(NC(=O)c2ccc(-n3ccnc3)nn2)cc1OCCc1cc(C(=O)O)c(NC(=O)c2ccc(-n3ccnc3)nn2)cc1Cl] |
| 157 | 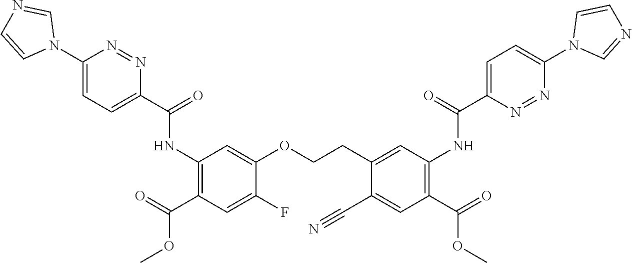 [SMILES: COC(=O)c1cc(F)c(OCCc2cc(NC(=O)c3ccc(-n4ccnc4)nn3)c(C(=O)OC)cc2C#N)cc1NC(=O)c1ccc(-n2ccnc2)nn1] |
| 158 | 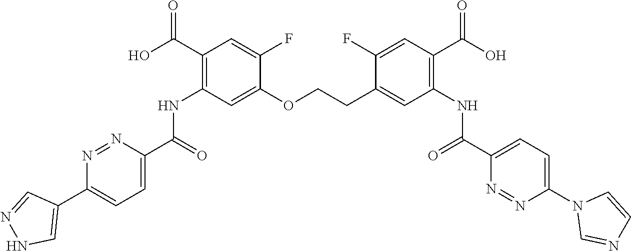 [SMILES: O=C(Nc1cc(OCCc2cc(NC(=O)c3ccc(-n4ccnc4)nn3)c(C(=O)O)cc2F)c(F)cc1C(=O)O)c1ccc(-c2cn[nH]c2)nn1] |
| 159 | 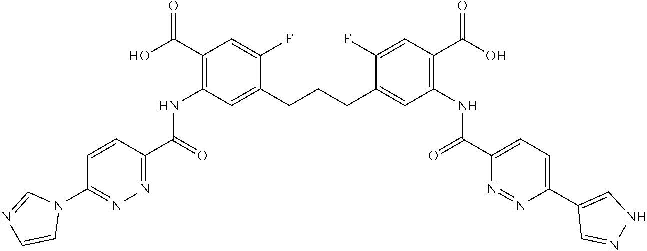 [SMILES: O=C(Nc1cc(CCCc2cc(NC(=O)c3ccc(-n4ccnc4)nn3)c(C(=O)O)cc2F)c(F)cc1C(=O)O)c1ccc(-c2cn[nH]c2)nn1] |
| 160 | 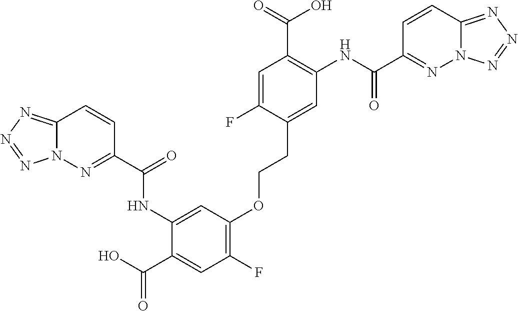 [SMILES: O=C(Nc1cc(CCOc2cc(NC(=O)c3ccc4nnnn4n3)c(C(=O)O)cc2F)c(F)cc1C(=O)O)c1ccc2nnnn2n1] |
| 161 | 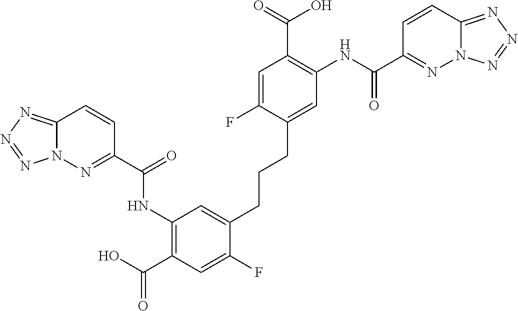 [SMILES: O=C(Nc1cc(CCCc2cc(NC(=O)c3ccc4nnnn4n3)c(C(=O)O)cc2F)c(F)cc1C(=O)O)c1ccc2nnnn2n1] |
| 162 | 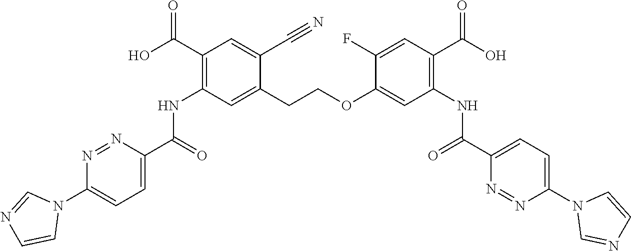 [SMILES: N#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] |
| 163 | 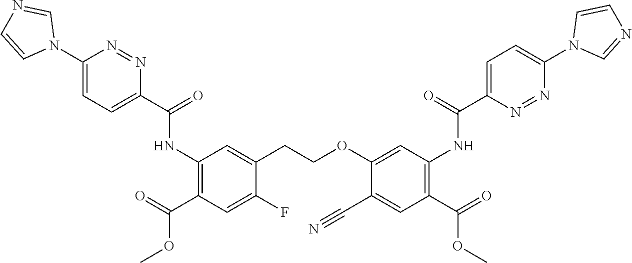 [SMILES: COC(=O)c1cc(F)c(CCOc2cc(NC(=O)c3ccc(-n4ccnc4)nn3)c(C(=O)OC)cc2C#N)cc1NC(=O)c1ccc(-n2ccnc2)nn1] |
| 164 | 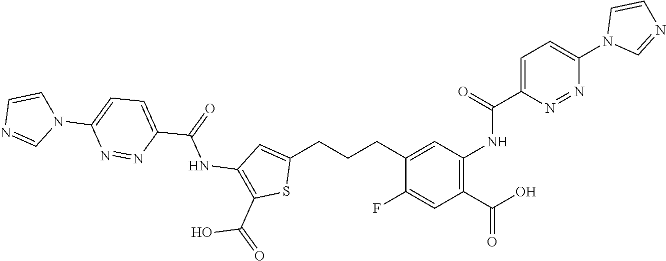 [SMILES: O=C(Nc1cc(CCCc2cc(NC(=O)c3ccc(-n4ccnc4)nn3)c(C(=O)O)s2)c(F)cc1C(=O)O)c1ccc(-n2ccnc2)nn1] |
| 165 | 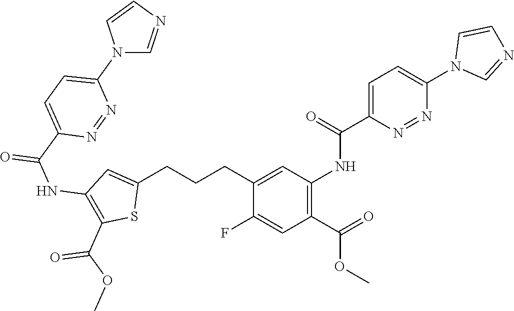 [SMILES: COC(=O)c1cc(F)c(CCCc2cc(NC(=O)c3ccc(-n4ccnc4)nn3)c(C(=O)OC)s2)cc1NC(=O)c1ccc(-n2ccnc2)nn1] |
| 166 | 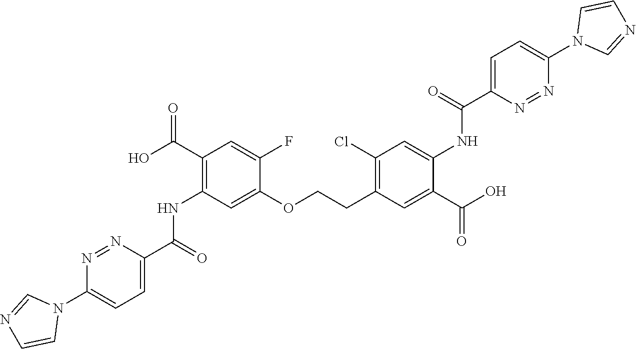 [SMILES: O=C(Nc1cc(OCCc2cc(C(=O)O)c(NC(=O)c3ccc(-n4ccnc4)nn3)cc2Cl)c(F)cc1C(=O)O)c1ccc(-n2ccnc2)nn1] |
| 167 | 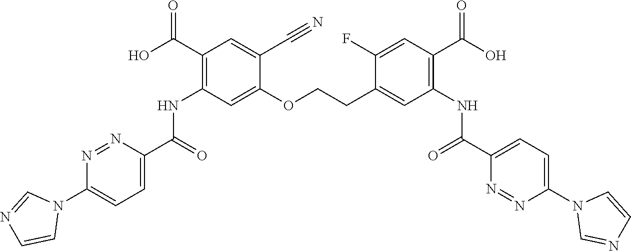 [SMILES: N#Cc1cc(C(=O)O)c(NC(=O)c2ccc(-n3ccnc3)nn2)cc1OCCc1cc(NC(=O)c2ccc(-n3ccnc3)nn2)c(C(=O)O)cc1F] |
| 168 | 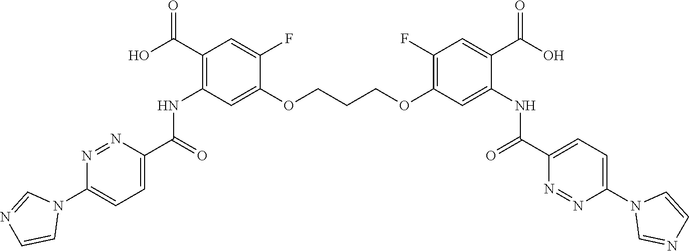 [SMILES: O=C(Nc1cc(OCCCOc2cc(NC(=O)c3ccc(-n4ccnc4)nn3)c(C(=O)O)cc2F)c(F)cc1C(=O)O)c1ccc(-n2ccnc2)nn1] |
| 169 | 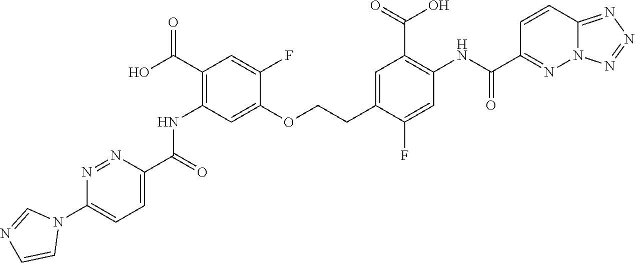 [SMILES: O=C(Nc1cc(OCCc2cc(C(=O)O)c(NC(=O)c3ccc4nnnn4n3)cc2F)c(F)cc1C(=O)O)c1ccc(-n2ccnc2)nn1] |
| 170 | 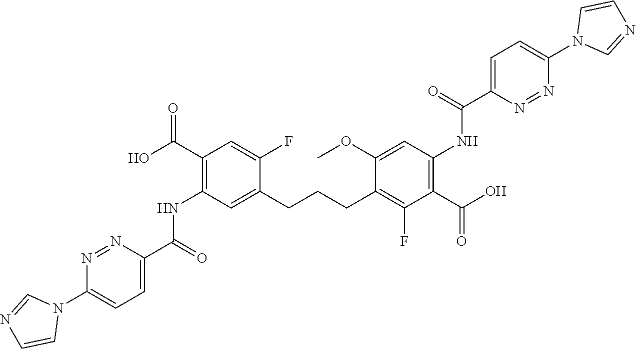 [SMILES: COc1cc(NC(=O)c2ccc(-n3ccnc3)nn2)c(C(=O)O)c(F)c1CCCc1cc(NC(=O)c2ccc(-n3ccnc3)nn2)c(C(=O)O)cc1F] |
| 171 | 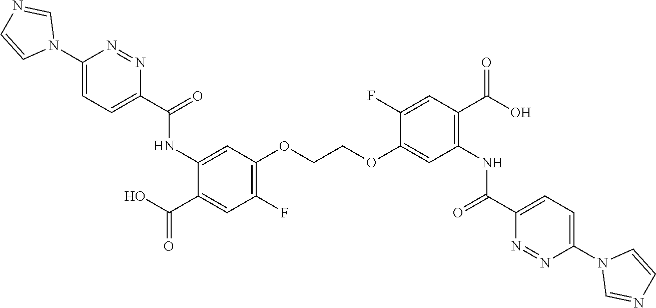 [SMILES: O=C(Nc1cc(OCCOc2cc(NC(=O)c3ccc(-n4ccnc4)nn3)c(C(=O)O)cc2F)c(F)cc1C(=O)O)c1ccc(-n2ccnc2)nn1] |
| 172 | 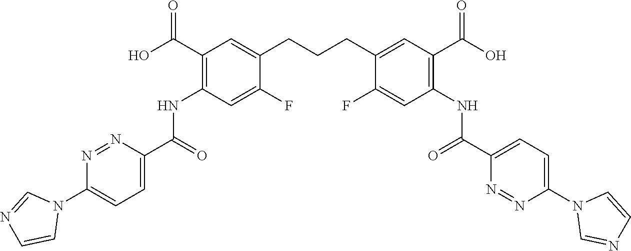 [SMILES: O=C(Nc1cc(F)c(CCCc2cc(C(=O)O)c(NC(=O)c3ccc(-n4ccnc4)nn3)cc2F)cc1C(=O)O)c1ccc(-n2ccnc2)nn1] |
| 173 | 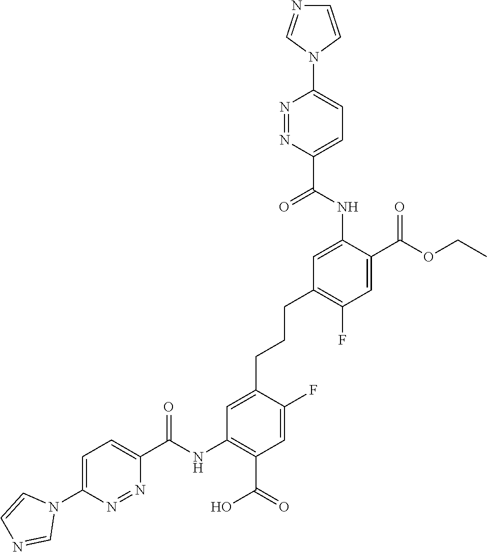 [SMILES: CCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] |
| 174 | 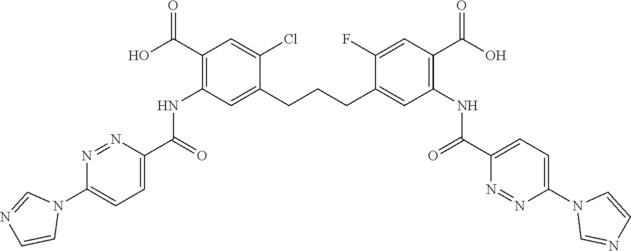 [SMILES: O=C(Nc1cc(CCCc2cc(NC(=O)c3ccc(-n4ccnc4)nn3)c(C(=O)O)cc2Cl)c(F)cc1C(=O)O)c1ccc(-n2ccnc2)nn1] |
| 175 | 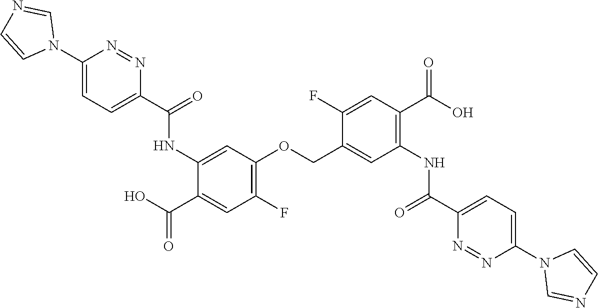 [SMILES: O=C(Nc1cc(COc2cc(NC(=O)c3ccc(-n4ccnc4)nn3)c(C(=O)O)cc2F)c(F)cc1C(=O)O)c1ccc(-n2ccnc2)nn1] |
| 176 | 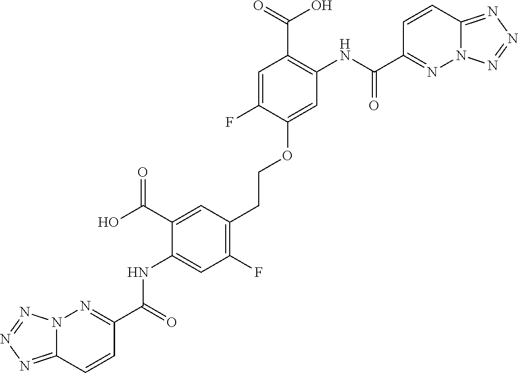 [SMILES: O=C(Nc1cc(OCCc2cc(C(=O)O)c(NC(=O)c3ccc4nnnn4n3)cc2F)c(F)cc1C(=O)O)c1ccc2nnnn2n1] |
| 177 | 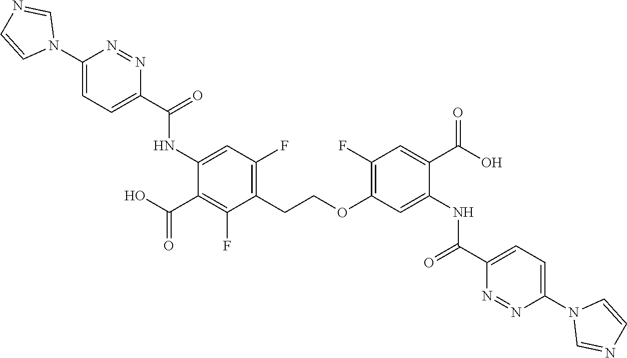 [SMILES: O=C(Nc1cc(OCCc2c(F)cc(NC(=O)c3ccc(-n4ccnc4)nn3)c(C(=O)O)c2F)c(F)cc1C(=O)O)c1ccc(-n2ccnc2)nn1] |
| 178 | 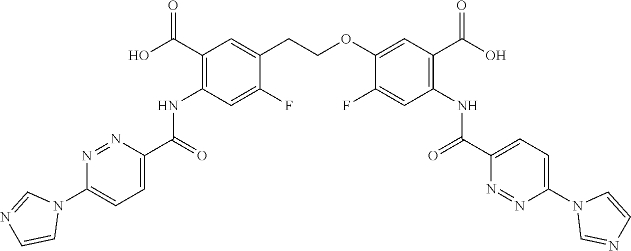 [SMILES: O=C(Nc1cc(F)c(CCOc2cc(C(=O)O)c(NC(=O)c3ccc(-n4ccnc4)nn3)cc2F)cc1C(=O)O)c1ccc(-n2ccnc2)nn1] |
| 179 | 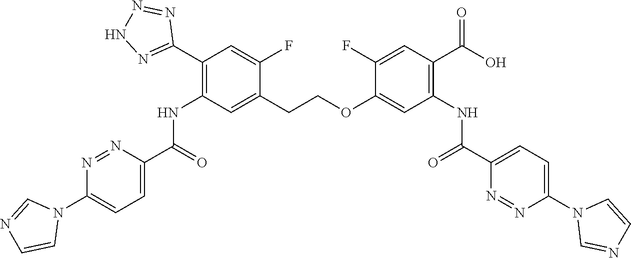 [SMILES: O=C(Nc1cc(OCCc2cc(NC(=O)c3ccc(-n4ccnc4)nn3)c(-c3nn[nH]n3)cc2F)c(F)cc1C(=O)O)c1ccc(-n2ccnc2)nn1] |
| 180 | 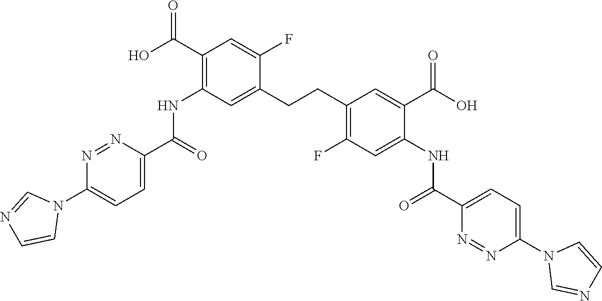 [SMILES: O=C(Nc1cc(CCc2cc(C(=O)O)c(NC(=O)c3ccc(-n4ccnc4)nn3)cc2F)c(F)cc1C(=O)O)c1ccc(-n2ccnc2)nn1] |
| 181 | 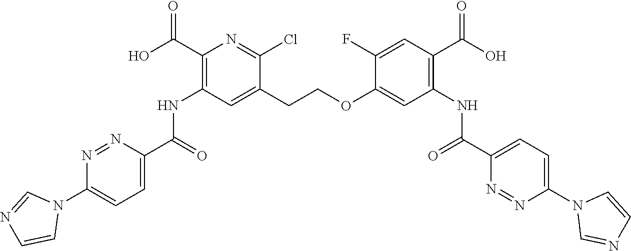 [SMILES: O=C(Nc1cc(OCCc2cc(NC(=O)c3ccc(-n4ccnc4)nn3)c(C(=O)O)nc2Cl)c(F)cc1C(=O)O)c1ccc(-n2ccnc2)nn1] |
| 182 | 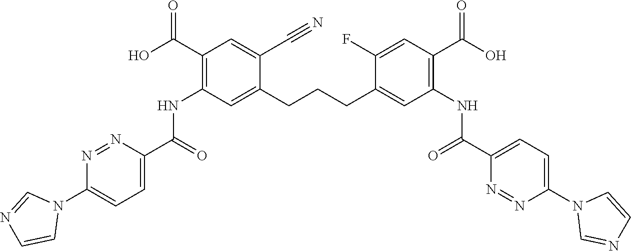 [SMILES: N#Cc1cc(C(=O)O)c(NC(=O)c2ccc(-n3ccnc3)nn2)cc1CCCc1cc(NC(=O)c2ccc(-n3ccnc3)nn2)c(C(=O)O)cc1F] |
| 183 | 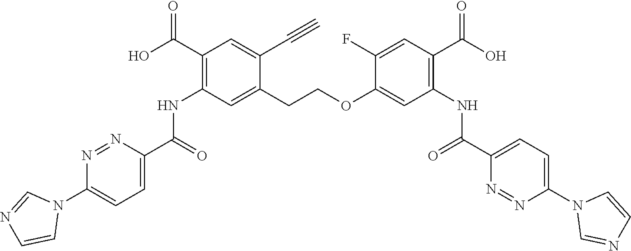 [SMILES: C#Cc1cc(C(=O)O)c(NC(=O)c2ccc(-n3ccnc3)nn2)cc1CCOc1cc(NC(=O)c2ccc(-n3ccnc3)nn2)c(C(=O)O)cc1F] |
| 184 | 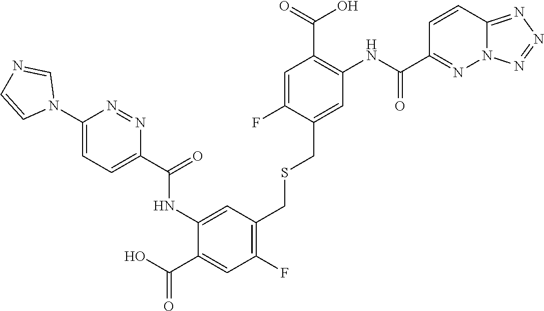 [SMILES: O=C(Nc1cc(CSCc2cc(NC(=O)c3ccc4nnnn4n3)c(C(=O)O)cc2F)c(F)cc1C(=O)O)c1ccc(-n2ccnc2)nn1] |
| 185 | 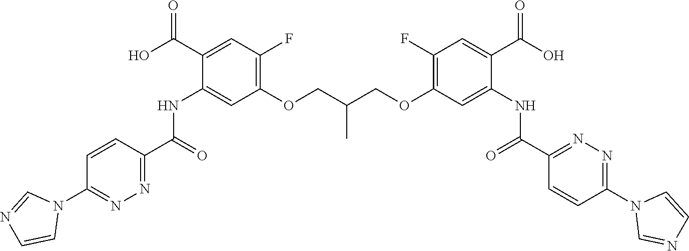 [SMILES: CC(COc1cc(NC(=O)c2ccc(-n3ccnc3)nn2)c(C(=O)O)cc1F)COc1cc(NC(=O)c2ccc(-n3ccnc3)nn2)c(C(=O)O)cc1F] |
| 186 | 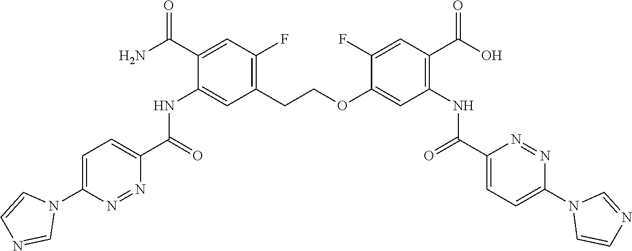 [SMILES: NC(=O)c1cc(F)c(CCOc2cc(NC(=O)c3ccc(-n4ccnc4)nn3)c(C(=O)O)cc2F)cc1NC(=O)c1ccc(-n2ccnc2)nn1] |
| 187 | 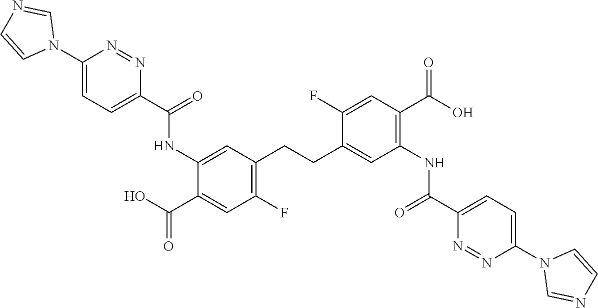 [SMILES: O=C(Nc1cc(CCc2cc(NC(=O)c3ccc(-n4ccnc4)nn3)c(C(=O)O)cc2F)c(F)cc1C(=O)O)c1ccc(-n2ccnc2)nn1] |
| 188 | 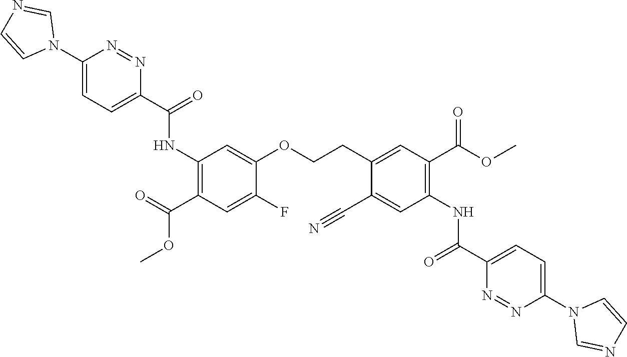 [SMILES: COC(=O)c1cc(F)c(OCCc2cc(C(=O)OC)c(NC(=O)c3ccc(-n4ccnc4)nn3)cc2C#N)cc1NC(=O)c1ccc(-n2ccnc2)nn1] |
| 189 | 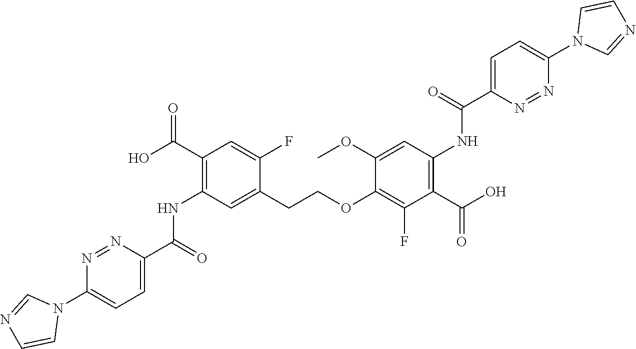 [SMILES: COc1cc(NC(=O)c2ccc(-n3ccnc3)nn2)c(C(=O)O)c(F)c1OCCc1cc(NC(=O)c2ccc(-n3ccnc3)nn2)c(C(=O)O)cc1F] |
| 190 | 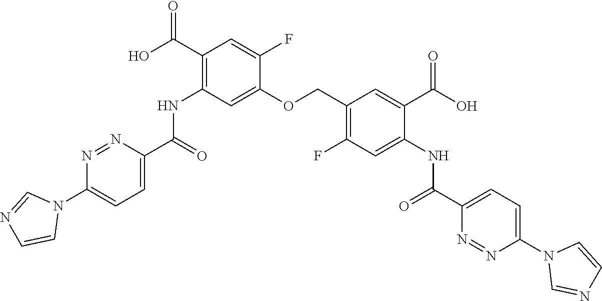 [SMILES: O=C(Nc1cc(OCc2cc(C(=O)O)c(NC(=O)c3ccc(-n4ccnc4)nn3)cc2F)c(F)cc1C(=O)O)c1ccc(-n2ccnc2)nn1] |
| 191 | 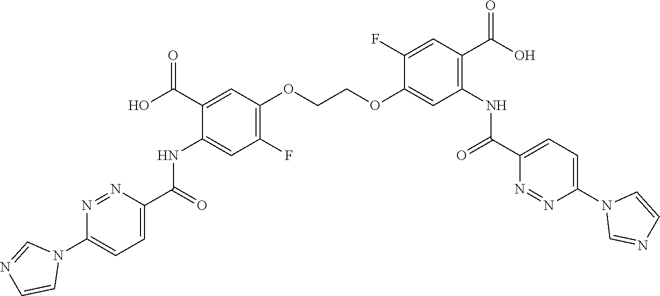 [SMILES: O=C(Nc1cc(OCCOc2cc(C(=O)O)c(NC(=O)c3ccc(-n4ccnc4)nn3)cc2F)c(F)cc1C(=O)O)c1ccc(-n2ccnc2)nn1] |
| 192 | 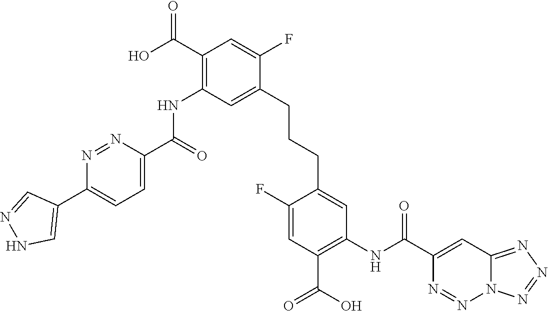 [SMILES: O=C(Nc1cc(CCCc2cc(NC(=O)c3cc4nnnn4nn3)c(C(=O)O)cc2F)c(F)cc1C(=O)O)c1ccc(-c2cn[nH]c2)nn1] |
| 193 | 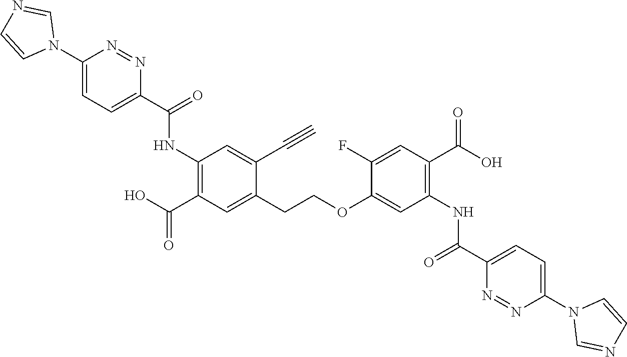 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1F] |
| 194 | 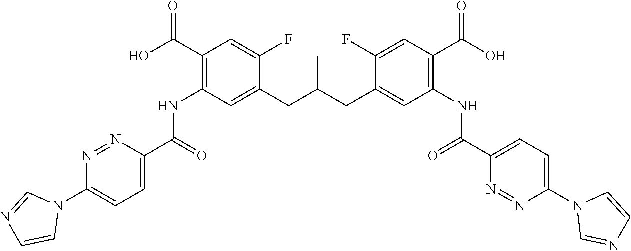 [SMILES: CC(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] |
| 195 | 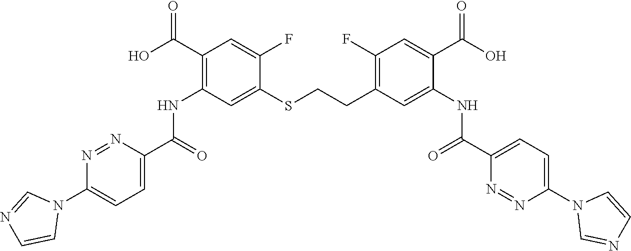 [SMILES: O=C(Nc1cc(CCSc2cc(NC(=O)c3ccc(-n4ccnc4)nn3)c(C(=O)O)cc2F)c(F)cc1C(=O)O)c1ccc(-n2ccnc2)nn1] |
| 196 | 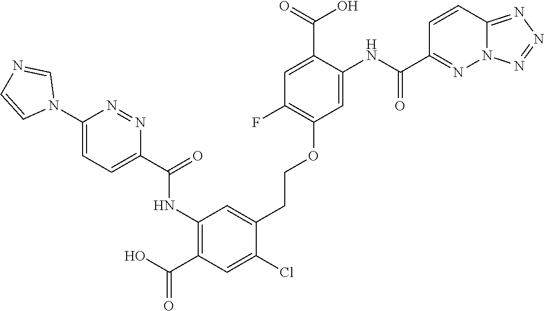 [SMILES: O=C(Nc1cc(CCOc2cc(NC(=O)c3ccc4nnnn4n3)c(C(=O)O)cc2F)c(Cl)cc1C(=O)O)c1ccc(-n2ccnc2)nn1] |
| 197 | 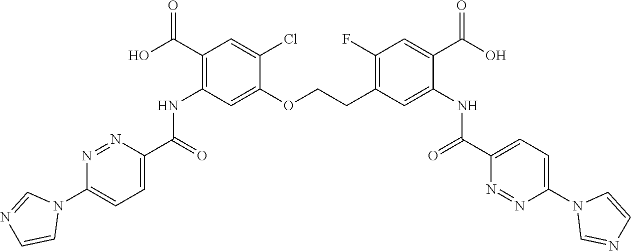 [SMILES: O=C(Nc1cc(CCOc2cc(NC(=O)c3ccc(-n4ccnc4)nn3)c(C(=O)O)cc2Cl)c(F)cc1C(=O)O)c1ccc(-n2ccnc2)nn1] |
| 198 | 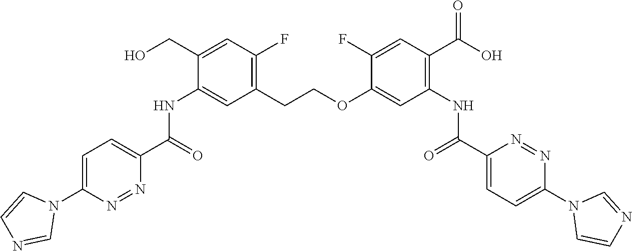 [SMILES: O=C(Nc1cc(CCOc2cc(NC(=O)c3ccc(-n4ccnc4)nn3)c(C(=O)O)cc2F)c(F)cc1CO)c1ccc(-n2ccnc2)nn1] |
| 199 | 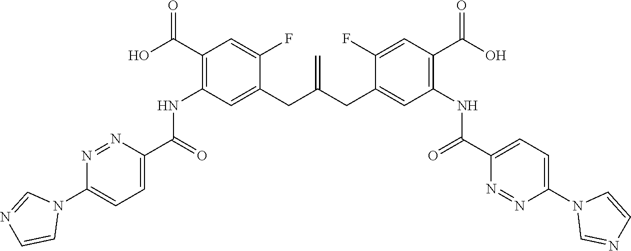 [SMILES: C=C(Cc1cc(NC(=O)c2ccc(-n3ccnc3)nn2)c(C(=O)O)cc1F)Cc1cc(NC(=O)c2ccc(-n3ccnc3)nn2)c(C(=O)O)cc1F] |
| 200 | 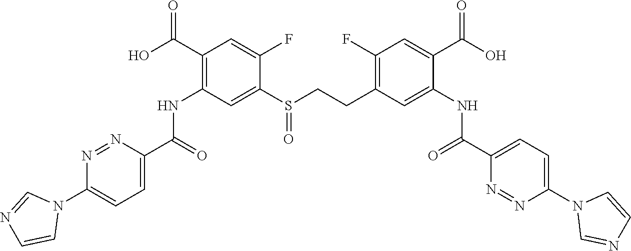 [SMILES: O=C(Nc1cc(CCS(=O)c2cc(NC(=O)c3ccc(-n4ccnc4)nn3)c(C(=O)O)cc2F)c(F)cc1C(=O)O)c1ccc(-n2ccnc2)nn1] |
| 201 | 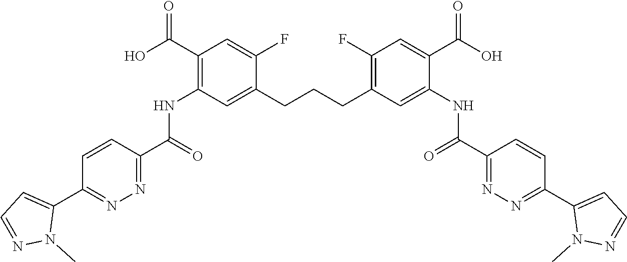 [SMILES: Cn1nccc1-c1ccc(C(=O)Nc2cc(CCCc3cc(NC(=O)c4ccc(-c5ccnn5C)nn4)c(C(=O)O)cc3F)c(F)cc2C(=O)O)nn1] |
| 202 | 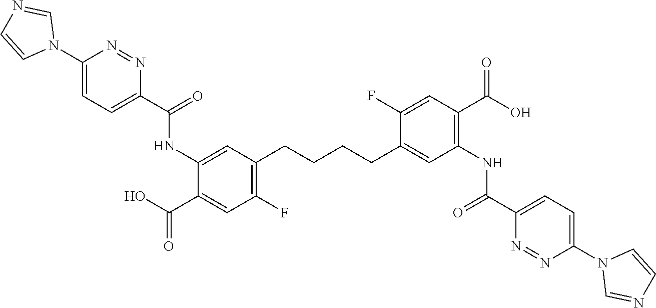 [SMILES: O=C(Nc1cc(CCCCc2cc(NC(=O)c3ccc(-n4ccnc4)nn3)c(C(=O)O)cc2F)c(F)cc1C(=O)O)c1ccc(-n2ccnc2)nn1] |
| 203 | 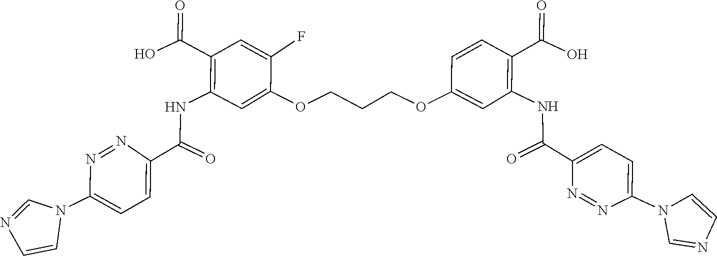 [SMILES: O=C(Nc1cc(OCCCOc2cc(NC(=O)c3ccc(-n4ccnc4)nn3)c(C(=O)O)cc2F)ccc1C(=O)O)c1ccc(-n2ccnc2)nn1] |
| 204 | 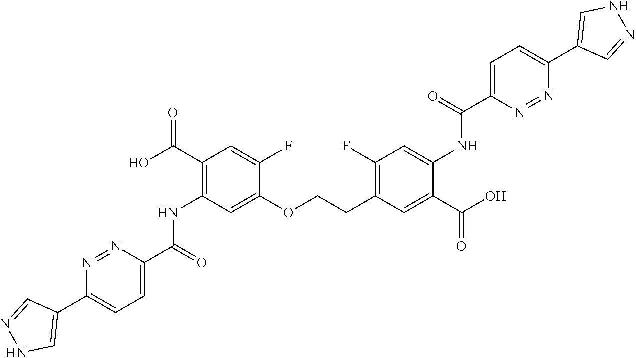 [SMILES: O=C(Nc1cc(OCCc2cc(C(=O)O)c(NC(=O)c3ccc(-c4cn[nH]c4)nn3)cc2F)c(F)cc1C(=O)O)c1ccc(-c2cn[nH]c2)nn1] |
| 205 | 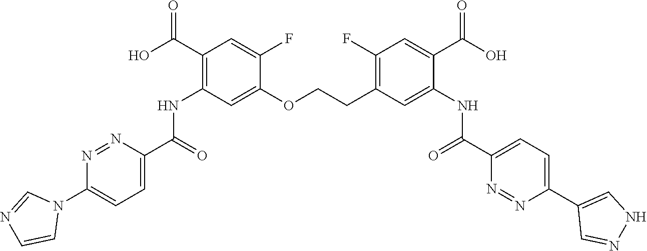 [SMILES: O=C(Nc1cc(CCOc2cc(NC(=O)c3ccc(-n4ccnc4)nn3)c(C(=O)O)cc2F)c(F)cc1C(=O)O)c1ccc(-c2cn[nH]c2)nn1] |
| 206 | 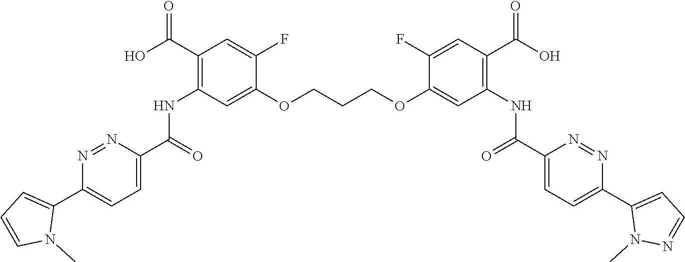 [SMILES: Cn1cccc1-c1ccc(C(=O)Nc2cc(OCCCOc3cc(NC(=O)c4ccc(-c5ccnn5C)nn4)c(C(=O)O)cc3F)c(F)cc2C(=O)O)nn1] |
| 207 | 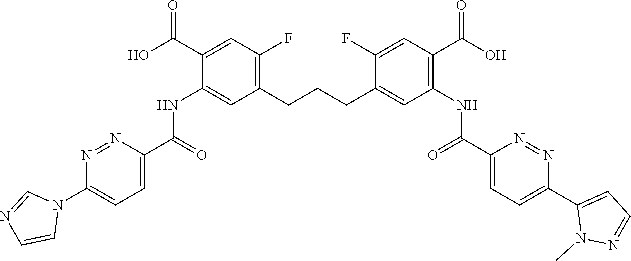 [SMILES: Cn1nccc1-c1ccc(C(=O)Nc2cc(CCCc3cc(NC(=O)c4ccc(-n5ccnc5)nn4)c(C(=O)O)cc3F)c(F)cc2C(=O)O)nn1] |
| 208 | 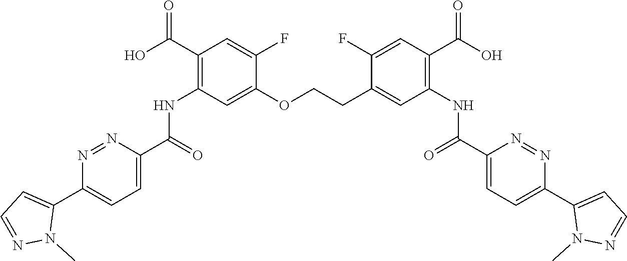 [SMILES: Cn1nccc1-c1ccc(C(=O)Nc2cc(CCOc3cc(NC(=O)c4ccc(-c5ccnn5C)nn4)c(C(=O)O)cc3F)c(F)cc2C(=O)O)nn1] |
| 209 | 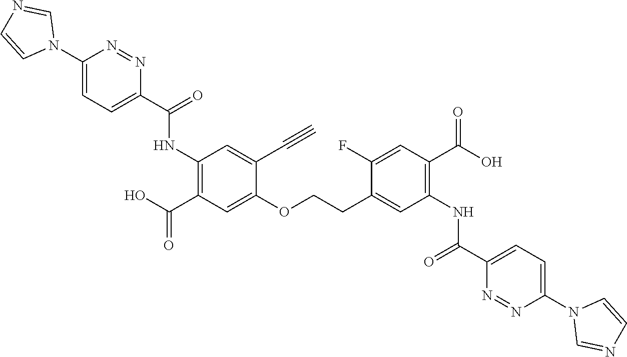 [SMILES: C#Cc1cc(NC(=O)c2ccc(-n3ccnc3)nn2)c(C(=O)O)cc1OCCc1cc(NC(=O)c2ccc(-n3ccnc3)nn2)c(C(=O)O)cc1F] |
| 210 | 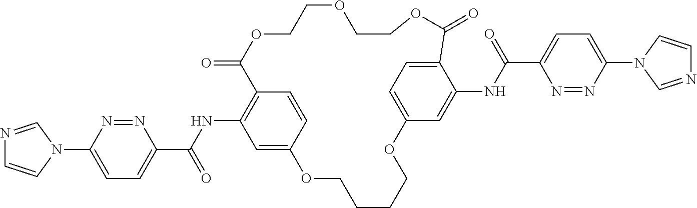 [SMILES: O=C(Nc1cc2ccc1C(=O)OCCOCCOC(=O)c1ccc(cc1NC(=O)c1ccc(-n3ccnc3)nn1)OCCCCO2)c1ccc(-n2ccnc2)nn1] |
| 211 | 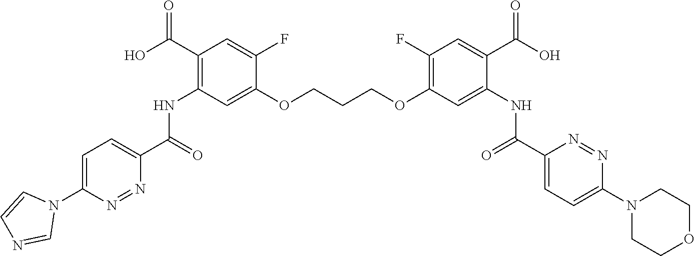 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCOCC2)nn1] |
| 212 | 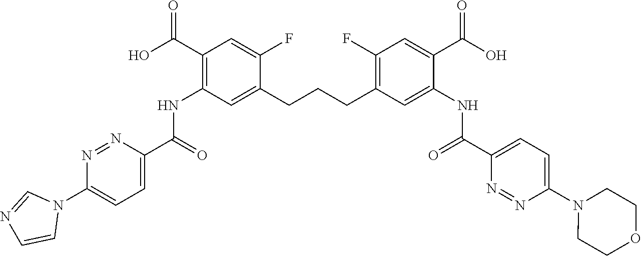 [SMILES: O=C(Nc1cc(CCCc2cc(NC(=O)c3ccc(-n4ccnc4)nn3)c(C(=O)O)cc2F)c(F)cc1C(=O)O)c1ccc(N2CCOCC2)nn1] |
| 213 | 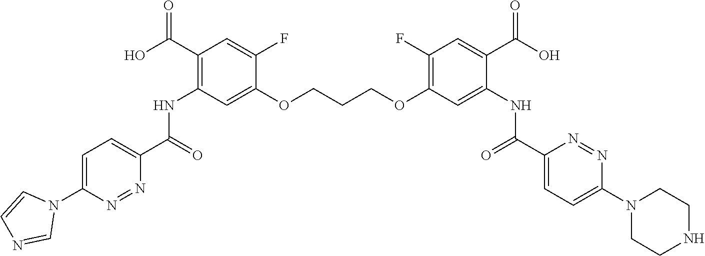 [SMILES: O=C(Nc1cc(OCCCOc2cc(NC(=O)c3ccc(-n4ccnc4)nn3)c(C(=O)O)cc2F)c(F)cc1C(=O)O)c1ccc(N2CCNCC2)nn1] |
| 214 | 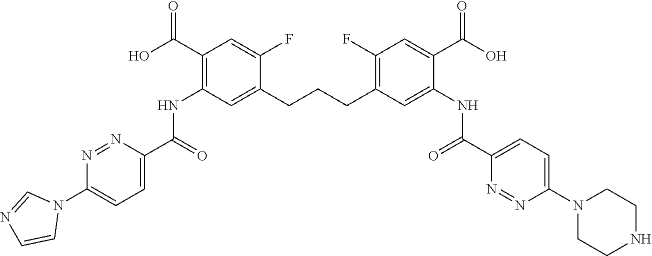 [SMILES: O=C(Nc1cc(CCCc2cc(NC(=O)c3ccc(-n4ccnc4)nn3)c(C(=O)O)cc2F)c(F)cc1C(=O)O)c1ccc(N2CCNCC2)nn1] |
| 215 | 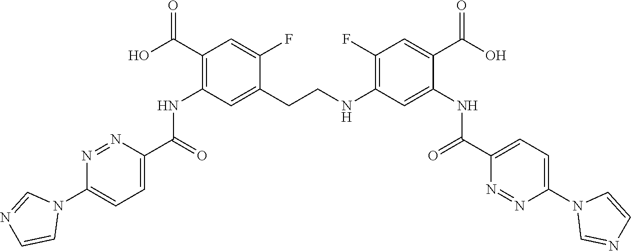 [SMILES: O=C(Nc1cc(CCNc2cc(NC(=O)c3ccc(-n4ccnc4)nn3)c(C(=O)O)cc2F)c(F)cc1C(=O)O)c1ccc(-n2ccnc2)nn1] |
| 216 | 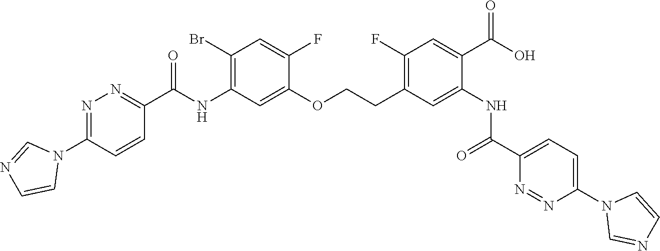 [SMILES: O=C(Nc1cc(OCCc2cc(NC(=O)c3ccc(-n4ccnc4)nn3)c(C(=O)O)cc2F)c(F)cc1Br)c1ccc(-n2ccnc2)nn1] |
| 217 | 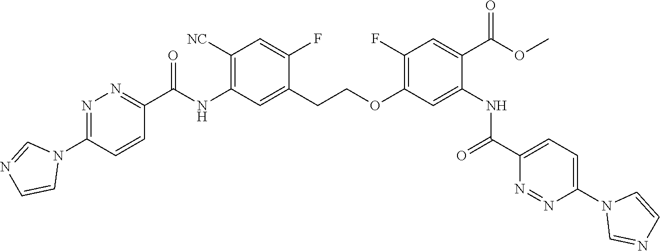 [SMILES: COC(=O)c1cc(F)c(OCCc2cc(NC(=O)c3ccc(-n4ccnc4)nn3)c(C#N)cc2F)cc1NC(=O)c1ccc(-n2ccnc2)nn1] |
| 218 | 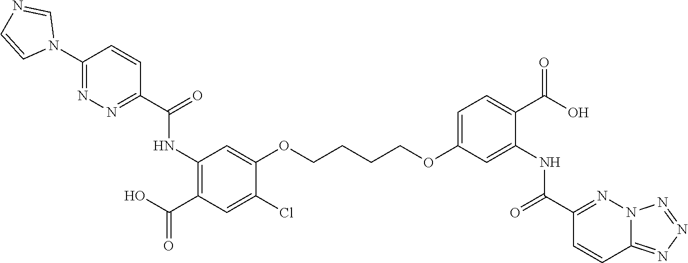 [SMILES: O=C(Nc1cc(OCCCCOc2ccc(C(=O)O)c(NC(=O)c3ccc4nnnn4n3)c2)c(Cl)cc1C(=O)O)c1ccc(-n2ccnc2)nn1] |
| 219 | 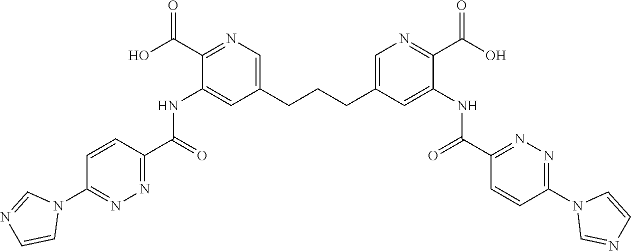 [SMILES: O=C(Nc1cc(CCCc2cnc(C(=O)O)c(NC(=O)c3ccc(-n4ccnc4)nn3)c2)cnc1C(=O)O)c1ccc(-n2ccnc2)nn1] |
| 220 | 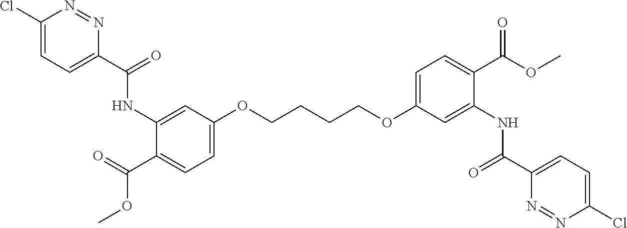 [SMILES: COC(=O)c1ccc(OCCCCOc2ccc(C(=O)OC)c(NC(=O)c3ccc(Cl)nn3)c2)cc1NC(=O)c1ccc(Cl)nn1] |
| 221 | 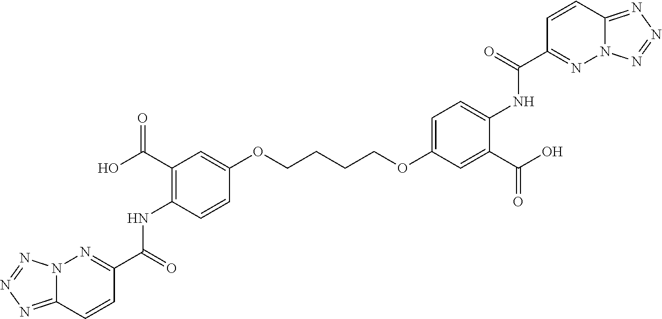 [SMILES: O=C(Nc1ccc(OCCCCOc2ccc(NC(=O)c3ccc4nnnn4n3)c(C(=O)O)c2)cc1C(=O)O)c1ccc2nnnn2n1] |
| 222 | 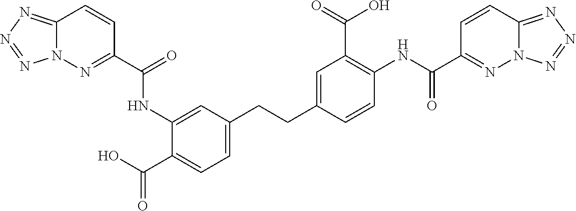 [SMILES: O=C(Nc1cc(CCc2ccc(NC(=O)c3ccc4nnnn4n3)c(C(=O)O)c2)ccc1C(=O)O)c1ccc2nnnn2n1] |
| 223 | 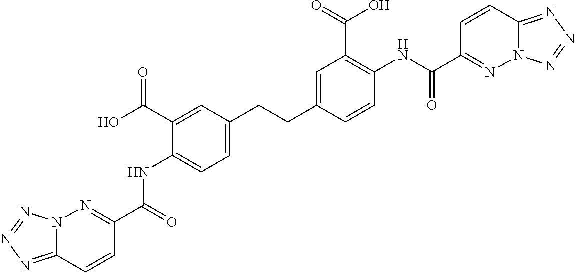 [SMILES: O=C(Nc1ccc(CCc2ccc(NC(=O)c3ccc4nnnn4n3)c(C(=O)O)c2)cc1C(=O)O)c1ccc2nnnn2n1] |
| 224 | 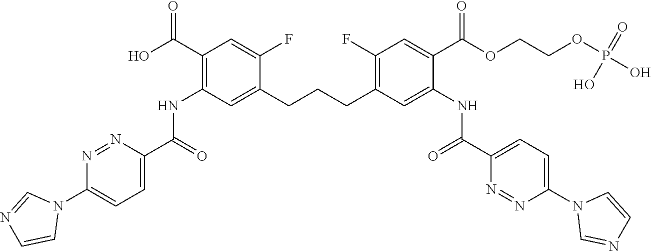 [SMILES: O=C(Nc1cc(CCCc2cc(NC(=O)c3ccc(-n4ccnc4)nn3)c(C(=O)OCCOP(=O)(O)O)cc2F)c(F)cc1C(=O)O)c1ccc(-n2ccnc2)nn1] |
| 225 | 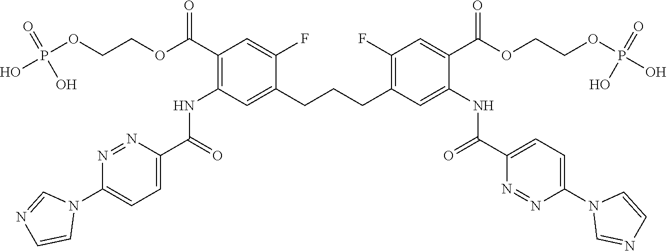 [SMILES: O=C(Nc1cc(CCCc2cc(NC(=O)c3ccc(-n4ccnc4)nn3)c(C(=O)OCCOP(=O)(O)O)cc2F)c(F)cc1C(=O)OCCOP(=O)(O)O)c1ccc(-n2ccnc2)nn1] |
| 226 | 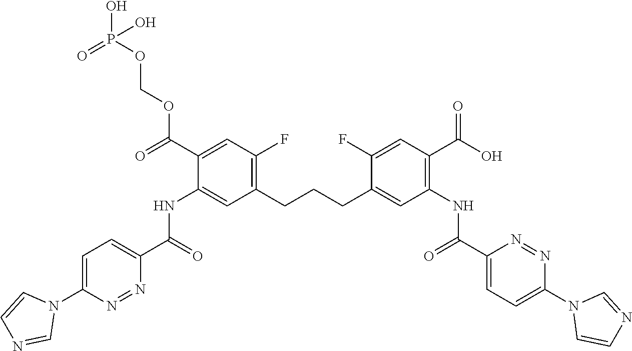 [SMILES: O=C(Nc1cc(CCCc2cc(NC(=O)c3ccc(-n4ccnc4)nn3)c(C(=O)OCOP(=O)(O)O)cc2F)c(F)cc1C(=O)O)c1ccc(-n2ccnc2)nn1] |
| 227 | 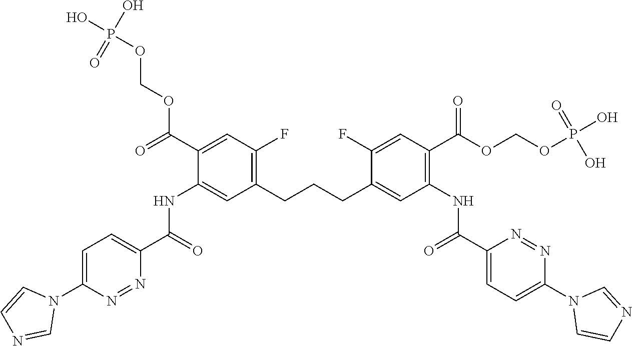 [SMILES: O=C(Nc1cc(CCCc2cc(NC(=O)c3ccc(-n4ccnc4)nn3)c(C(=O)OCOP(=O)(O)O)cc2F)c(F)cc1C(=O)OCOP(=O)(O)O)c1ccc(-n2ccnc2)nn1] |
| 228 | 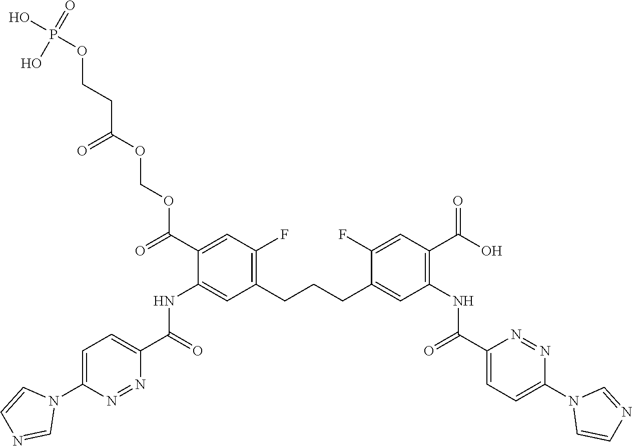 [SMILES: O=C(CCOP(=O)(O)O)OCOC(=O)c1cc(F)c(CCCc2cc(NC(=O)c3ccc(-n4ccnc4)nn3)c(C(=O)O)cc2F)cc1NC(=O)c1ccc(-n2ccnc2)nn1] |
| 229 | 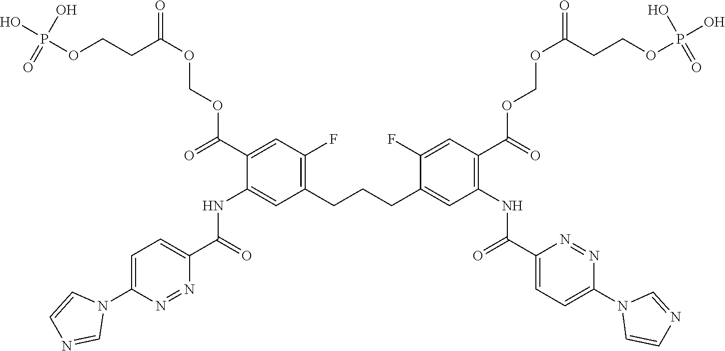 [SMILES: O=C(CCOP(=O)(O)O)OCOC(=O)c1cc(F)c(CCCc2cc(NC(=O)c3ccc(-n4ccnc4)nn3)c(C(=O)OCOC(=O)CCOP(=O)(O)O)cc2F)cc1NC(=O)c1ccc(-n2ccnc2)nn1] |
| 230 | 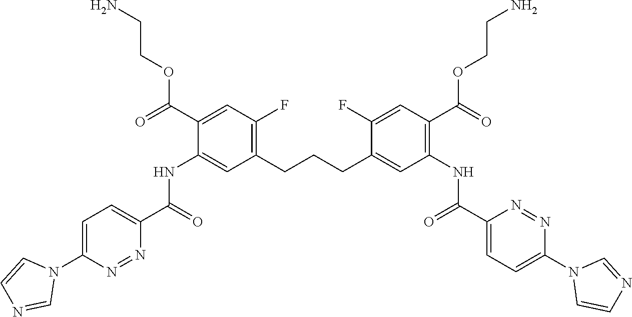 [SMILES: NCCOC(=O)c1cc(F)c(CCCc2cc(NC(=O)c3ccc(-n4ccnc4)nn3)c(C(=O)OCCN)cc2F)cc1NC(=O)c1ccc(-n2ccnc2)nn1] |
| 231 | 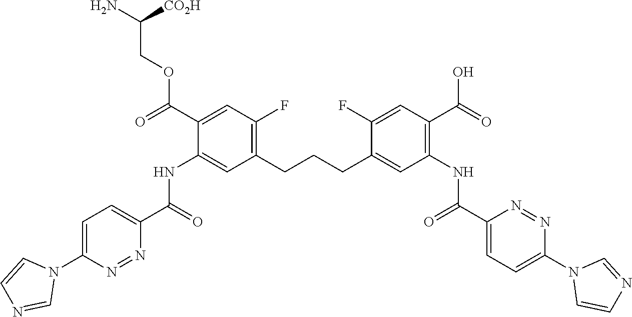 [SMILES: N[C@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] |
| 232 | 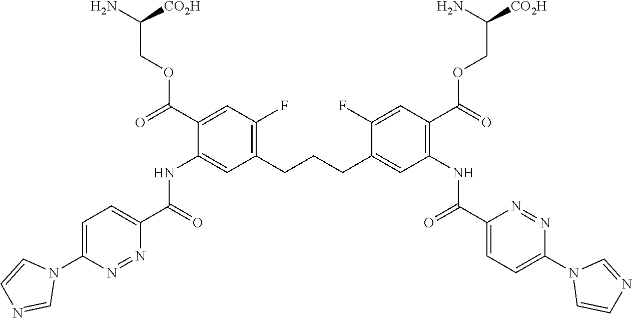 [SMILES: N[C@H](COC(=O)c1cc(F)c(CCCc2cc(NC(=O)c3ccc(-n4ccnc4)nn3)c(C(=O)OC[C@@H](N)C(=O)O)cc2F)cc1NC(=O)c1ccc(-n2ccnc2)nn1)C(=O)O] |
| 233 | 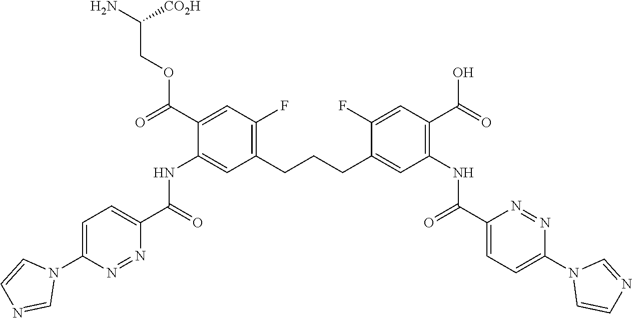 [SMILES: N[C@@H](COC(=O)c1cc(F)c(CCCc2cc(NC(=O)c3ccc(-n4ccnc4)nn3)c(C(=O)O)cc2F)cc1NC(=O)c1ccc(-n2ccnc2)nn1)C(=O)O] |
| 234 | 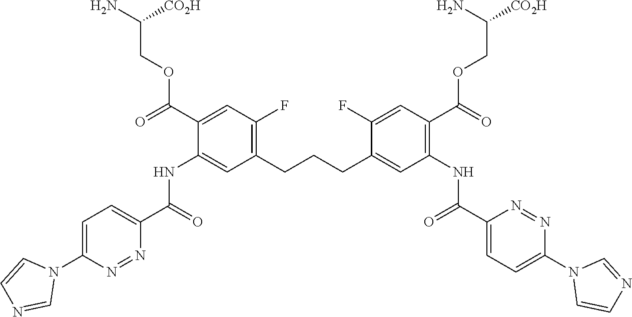 [SMILES: N[C@@H](COC(=O)c1cc(F)c(CCCc2cc(NC(=O)c3ccc(-n4ccnc4)nn3)c(C(=O)OC[C@H](N)C(=O)O)cc2F)cc1NC(=O)c1ccc(-n2ccnc2)nn1)C(=O)O] |
| 235 | 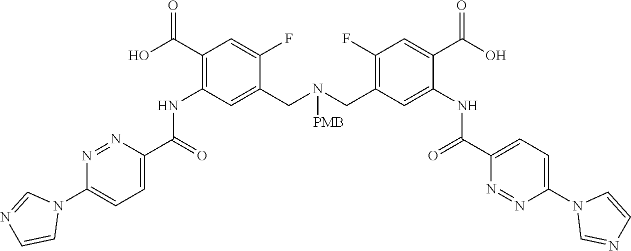 |
| 236 | 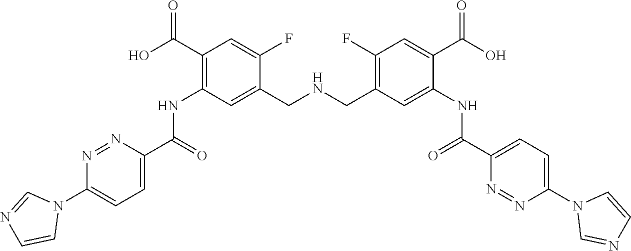 [SMILES: O=C(Nc1cc(CNCc2cc(NC(=O)c3ccc(-n4ccnc4)nn3)c(C(=O)O)cc2F)c(F)cc1C(=O)O)c1ccc(-n2ccnc2)nn1] |
| 237 | 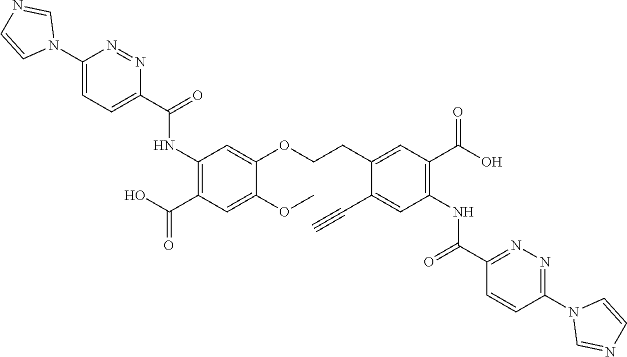 [SMILES: C#Cc1cc(NC(=O)c2ccc(-n3ccnc3)nn2)c(C(=O)O)cc1CCOc1cc(NC(=O)c2ccc(-n3ccnc3)nn2)c(C(=O)O)cc1OC] |
| 238 | 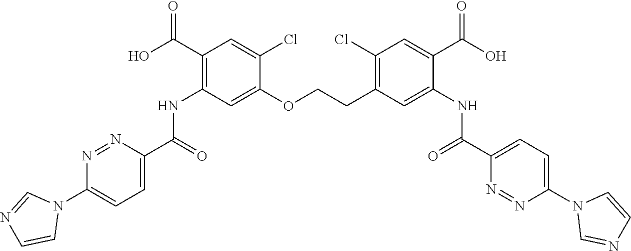 [SMILES: O=C(Nc1cc(CCOc2cc(NC(=O)c3ccc(-n4ccnc4)nn3)c(C(=O)O)cc2Cl)c(Cl)cc1C(=O)O)c1ccc(-n2ccnc2)nn1] |
| 239 | 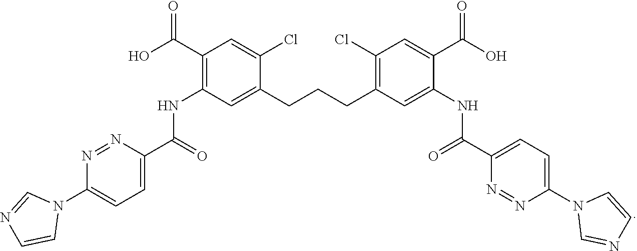 [SMILES: O=C(Nc1cc(CCCc2cc(NC(=O)c3ccc(-n4ccnc4)nn3)c(C(=O)O)cc2Cl)c(Cl)cc1C(=O)O)c1ccc(-n2ccnc2)nn1 ; "."] |

30. A pharmaceutical composition comprising a compound or pharmaceutically acceptable salt thereof according to any one of claims 1 to 29 and a pharmaceutically acceptable carrier.

31. A method of stimulating expression of interferon genes in a human patient, comprising administering to the patient an effective dose of a compound or pharmaceutically acceptable salt thereof according to any one of claims 1 to 29.

32. A method of treating a tumor in a patient, comprising administering to the patient an effective dose of a compound or pharmaceutically acceptable salt thereof according to any one of claims 1 to 29.

33. The method according to claim 31 or 32, wherein the administering comprises oral or intratumoral administration, or both.

34. The method according to claim 31 or 32, wherein administering comprises administering the compound to the patient as an antibody-drug conjugate or in a liposomal formulation.

35. The method according to claim 31 or 32, further comprising administering an effective amount of an immune-checkpoint targeting drug.

36. The method according to claim 35, wherein the immune-checkpoint targeting drug comprises an anti-PD-L1 antibody, anti-PD-1 antibody, anti-CTLA-4 antibody, or an anti-4-1BB antibody.

37. The method according to claim 31 or 32, further comprising administering ionizing radiation or anticancer drugs.

38. A compound or pharmaceutically acceptable salt thereof according to any one of claims 1 to 29 for use in a method of stimulating expression of interferon genes in a human patient.

39. A compound or pharmaceutically acceptable salt thereof according to any one of claims 1 to 29 for use in a method of treating a tumor in a patient.

40. The compound for use according to claim 38 or 39, wherein the compound is administered to the patient by oral or intratumoral administration, or both.
